(19) United States
(12) Patent Application Publication
(10) Pub. No.: US 2007/0161009 A1
(43) Pub. Date: Jul. 12, 2007

(54) METHOD FOR PRODUCING IMPROVED GENE EXPRESSION ANALYSIS AND GENE EXPRESSION ANALYSIS COMPARISON RESULTS

(76) Inventor: David E. Kohne, San Diego, CA (US)

Correspondence Address: WESLEY B. AMES, 7031 LOS VIENTOS SERENOS, ESCONDIDO, CA, 92029, US

(21) Appl. No.: 11/421,961

(22) Filed: Jun. 2, 2006

Related U.S. Application Data
(60) Provisional application No. 60/687,526, filed on Jun. 3, 2005.

(51) Int. Cl. C12Q 1/68; G06F 19/00; C12P 19/34

(52) U.S. Cl. 435/6; 435/91.2; 702/20

(57) ABSTRACT

This invention relates to methods and means for producing microarray, non-microarray and clone counting method gene expression and gene expression comparison assay results which are, relative to such prior art produced assay results, known to be significantly improved in normalization and/or assay accuracy and/or biological accuracy, and/or quantitation, and/or interpretability and/or intercomparability, and/or utility. The practice of the invention is necessary to produce microarray, non-microarray, and clone counting method assay measured gene expression and gene expression analysis assay results which can be known to be accurate.

### RELATED APPLICATIONS

This application claims the benefit of Kohne, U.S. Provisional Application 60/687,526, filed Jun. 8, 2005, which is incorporated herein by reference in its entirety.

### FIELD OF THE INVENTION

The present invention relates to the field of biological and biochemical in vitro assays, and especially to the field of nucleic acid based assays such as assays related to the determination and comparison expression levels of particular genes and creation and comparison of gene expression profiles.

### BACKGROUND OF THE INVENTION

The following discussion is provided solely to assist the understanding of the reader, and does not constitute an admission that any of the information discussed or references cited constitute prior art to the present invention.

In order to assist the reader, the following outline of the discussion of background materials is provided.

Background Outline
General Aspects of gene expression in cells
Natural differences in total RNA and Total mRNA content of cells
Polyadenylated mRNA
Gene expression analysis
Microarray and non-microarray gene expression analysis
Determination of a Microarray or non-microarray measured and normalized differential gene expression ratio (N-DGER)
Microarray and non-microarray gene expression comparison assay variables
Assumptions required for prior art normalization
Interpretation of positive and negative gene activity results
Current method for determining the relative amounts of cell sample nucleic acid compared in the assay
Current method for determining the relative amounts of cell sample cDNA or cRNA compared in the assay
Current method for determining the absolute amount of cell sample RNA or equivalents compared in the assay
Independent validation and corroboration of Microarray gene expression comparison results
Prior art considered assay variables associated with the normalization of prior art non-microarray gene expression analysis results.
Key prior art beliefs and practices for Microarray and non-microarray gene expression analysis.
Key prior art beliefs and practices for Microarray and non-microarray gene expression analysis. Three tacit assumptions. The representation and frequency of RNA transcripts and RNA transcript equivalents
Other key assumptions and prior art Microarray and non-microarray assay beliefs and practices
SAGE and other clone counting methods of gene expression analysis and comparison

General Aspects of Gene Expression in Cells.

At the most basic level, gene expression and changes in gene expression occur in a single cell (1). Within a cell, a variety of different endogenous chromosomal and extrachromosomal DNA genes are present. In a cell, these endogenous genes are transcribed into a wide variety of different RNA transcripts of nuclear, mitochondrial, or other extra-nuclear origin. Such RNAs include, but are not limited to, nuclear, mitochondrial, and cytoplasmic RNA transcripts of all kinds such as ribosomal RNA (rRNA), transfer RNA (tRNA), small interfering RNA (siRNA), micro-interfering RNA (miRNA), small nucleolar RNA (snoRNA), and other RNA (1, 2). DNA and/or RNA genes and other RNA types from infectious agents such as viruses, bacteria, and other cells, can also be present in a cell, and these genes often produce RNA transcripts. The presence of such exogenous DNA or RNA in a cell can be due to the natural infection of a cell by a DNA and/or RNA virus, infection by another cell, or a naturally occurring DNA or RNA transfection event. Endogenous and/or exogenous DNA and/or RNA, or an exogenous cell and/or RNA or DNA virus type may also be artificially introduced into a cell. Often a cell contains genes comprised of DNA, and genes comprised of RNA, and both types of genes can be transcribed into RNA in the cell.

In a cell certain endogenous genes and/or exogenous genes are expressed or transcribed into RNA and others are not, and the number of RNA transcripts present in the cell is higher for some genes than for others (1). It is important for understanding the function of a gene in a cell to know a quantitative measure of the degree or extent to which an RNA or DNA gene is expressed (3-6). In each cell or group of cells, a gene expression profile exists, and in a cell containing exogenous genes, the exogenous and endogenous combination profile reflects the overall gene expression profile. A gene expression profile for a cell sample should describe the genes which are expressed, i.e., active, and those which are not expressed, i.e., inactive, and should also provide a measure of the extent of expression or activity for each active gene in the cell or cell sample.

The primary focus of prior art gene expression studies has by far, concerned the study of the expression of mRNAs in eukaryote and prokaryote cells. The primary purpose of mRNA is to be translated into protein. Other types of RNA have other purposes, which have been well documented (12). In a cell, it appears that the vast majority of genes code for mRNA and protein. Other genes are present far less frequently. In mammals for example, it is estimated that 25,000 to 30,000 genes code for mRNA and protein, while the recently discovered class of natural antisense RNAs is coded for by about 2,500 to 3,000 genes. In addition, the current general consensus is that many other unknown genes, which make RNA, may be present in the mammalian DNA. Because the vast majority of gene expression analysis studies have involved cellular produced mRNAs, for simplicity herein, this document will primarily emphasize and discuss the cellular expression of mRNAs in a cell. However, these discussions are also directly applicable to other cellular produced known and unknown exogenous or endogenous RNA transcript and gene types, including but not limited to, rRNAs, tRNAs, miRNAs, siRNAs, and snoRNAs, as well as other known or unknown RNA types, such as viral RNAs.

The total number of mRNA molecules per cell is different in different cell types. The total number of mRNA molecules in a typical mammalian cell ranges from 1-10×10⁵, and the number of different mRNA molecule types present in a typical mammalian cell is around 12,000. Thus, about 12,000 different mRNA coding genes are expressed in a typical mammalian cell (1, 7, 8). The comparable figures for yeast and the bacteria E. coli, are about 15,000 mRNA molecules per cell for yeast, representing about 2,500 yeast mRNA genes (9), and about 1,400 polycistronic bacterial mRNAs per cell, representing about 3,000-4,000 different bacterial mRNA genes (10, 11).

An average mammalian cell is assumed to contain a total of about 300,000 mRNA transcript copies per cell and the mRNA population in each cell is composed of three abundance classes (1, 7, 8, 9). The abundance of a particular gene's mRNA in a cell is the number of copies of that mRNA which is present in each cell. The high abundance class contains those mRNA transcripts, which are present in thousands of copies per cell, and represents the expression of ten or so different genes. The intermediate abundance class contains mRNA transcripts, which are present in tens to hundreds of copies per cell, and represent the expression of hundreds of different genes. The low abundance class consists of mRNA transcripts which are present at around 1-20 copies per cell and represent the expression of 10,000 or so different genes. The copy per cell number for each abundance represents an average for the distribution present in that abundance class. In a mammalian cell's low abundance class, there are thousands of genes, which are expressed at levels from less than one copy per cell to five copies per cell (1, 7, 8, 9).

In different cells from the same organism, thousands of the same genes are active and produce low abundance mRNA transcripts. A comparison of mouse liver, kidney, and brain, low abundance mRNA transcripts indicated that liver and brain low abundance mRNA's each held in common over half of the kidney low abundance mRNA transcripts. The abundance of the mRNA transcripts held in common was similar, but not necessarily identical in each tissue (1). This large overlap between the mRNA populations of different cell types, including neoplastic cells, is common for mammals and other eukaryotes (1, 7, 8, 9). In different mammalian cell samples, it appears that thousands of the same genes in each sample are expressed at the same abundance level in each cell sample and the number of mRNA transcripts per cell for a gene in one cell sample, is equal to or near the number of the same gene's mRNA transcripts per cell in another cell sample.

Thus, in a comparison of the same genes which are present in different mammalian cell samples, little or no difference in abundance is believed to exist for thousands of different particular gene low abundance mRNA transcripts. Herein the comparison of the same particular genes RNA transcript expression in different cell samples is termed a same gene different cell sample expression comparison, or a SGDS comparison. Prior art assays virtually always do SGDS particular gene mRNA transcript comparisons. For such different mammalian cell sample comparisons, differences in mRNA transcript abundance often exist for a particular gene in one cell sample and a different particular gene in the compared other cell sample. Herein the comparison of the expression of one particular gene in one cell sample to the expression of a different particular gene in a different cell sample is termed a different gene different cell sample comparison, or a DGDS comparison. Prior art only rarely does DGDS mRNA transcript comparisons. As discussed above, different genes in the same cell or cell sample are expressed to different extents and are associated with different RNA transcript abundance levels in the same cell or cell sample. Herein, the comparison of the extents of expression of two different particular genes RNA transcripts which are present in the same cell or cell sample is termed a different gene same cell sample comparison, or a DGSS comparison. Prior art only rarely does DGSS mRNA transcript comparisons.

Differences in gene expression are responsible for structural, chemical, and behavioral differences between cells. Differences in gene expression, also termed Differential Gene Expression (DGE), can be identified by comparing individual gene expression profiles from different cell samples (3-8). A DGE profile, resulting from the comparison of two separate gene expression profiles should provide information on two aspects of cellular gene expression. First, whether a gene is expressed in both cell samples. Second, a quantitative measure of the number of molecules per cell in each different cell sample for each particular gene's RNA transcripts. A complete DGE profile for a cell sample comparison thus requires SGDS, DGDS, and DGSS, comparisons.

In the event of a change in a gene's extent of expression, the number of RNA transcripts per cell may be increased (upregulated), or decreased (downregulated), or may remain unchanged (unregulated). It is important to know both the magnitude and direction of a change (12). Since almost all gene expression measurements involve one or more populations of cells, the gene expression measurements are averages for the population, and do not necessarily reflect the actual situation in any one cell.

Natural Differences in the Total RNA and Total mRNA Content of Cells.

It has long been known that the total RNA content of individual prokaryotic and eukaryotic cells can vary greatly, depending on their type, state of differentiation and growth, and environment. The total RNA content of rapid growing bacterial cells is reported to be ten times higher than that for slow growing cells (10, 11). The amount of total cytoplasmic RNA obtained from different types of mammalian tissue culture cells varies greatly, from 30 micrograms per 10⁷ cells, to 500 micrograms per 10⁷ cells, depending on the cell sizes and state of differentiation (13). Mouse 3T3 or 3T6 cultured fibroblast cells, which are growing, have been reported to have a fourfold higher total RNA content than non-growing cells (1, 14).

Similarly, it has long been known that the total RNA contents of different cell types present in one eukaryotic organism are different, and that the same cells at different stages of differentiation can have different total RNA contents. A convenient method for estimating the difference in total RNA content in different cells is to compare the total RNA/DNA ratio of the cells or tissues. Adult rat or mouse liver cells have an RNA/DNA ratio, which is about twenty-five fold larger than rat and mouse thymus cells (15). The actual difference in RNA content per cell may depend on the DNA content of the average liver or thymus cell (or the average ploidy). Taking this into account, the RNA content difference could, in theory, range from 12-50 fold. Adult rat liver has a total RNA/DNA ratio, which is about three times that of rat fetal liver (15). In this case, the RNA content per diploid cell difference could range from 1.5 to 6 fold. It has been reported that adult rat liver cells have an RNA content which is about three times greater than the cells of a neoplastic rat hepatoma tumor (15). Here the RNA content per cell could vary from 1.5 to 6 fold. There are also reports that there are significant differences in the RNA contents per cell of the same cell types present in different mammalian species (15).

Table 1 presents a summary of published average RNA/DNA ratios per cell for different rat cell or tissue types (15). Reference (15) contains RNA/DNA ratios for different cells or tissue from a variety of different eukaryotes and mammals. Overall, there is a lack of data on total RNA content per cell for cells and tissues under varied conditions. Some information is available in the catalogs of companies, which sell purified total RNA and mRNA. These RNA/DNA ratios are generally consistent with those presented in Table 1 and reference (15). See, for example, the Qiagen 2001 catalog, page 297.

TABLE 1
Total RNA/DNA Ratios for Various Rat Cells or Tissues (15)

| | Developmental | | Range of Ratio |
|---|---|---|---|
| Cell or Tissue | Stage | Total RNA/DNA Ratio | Measurements |
| Liver | Adult | 4.3 (n = 5) | 3.28-5.14 |
| Thymus | Adult | 0.17 (n = 3) | 0.14-0.19 |
| Pancreas | Adult | 4 (n = 3) | 3.96-4.1 |
| Brain | Adult | 1.6 (n = 4) | 0.94-2.67 |
| Lung | Adult | 0.49 (n = 3) | 0.3-0.57 |
| Bone Marrow | Adult | 0.7 (n = 3) | 0.57-0.97 |
| Heart | Adult | 0.97 (n = 3) | 0.85-1.03 |
| Hepatoma | Adult | 1.14 (n = 3) | 0.81-1.32 |
| Liver | Adult | 4.5 (n = 3) | 4.21-4.62 |
| Liver | Fetal | 1.3 (n = 3) | 0.93-1.94 |

n = Number of different determinations

Only a small fraction of the total RNA in a cell or tissue consists of mRNA transcripts. A common method of describing the amount of total mRNA present in the total RNA of a cell sample is to designate the percent of total RNA which consists of total mRNA. For mammals and other eukaryotes, the amount of total RNA, which consists of poly A mRNA, is regarded as being the total mRNA fraction. This is believed to be close to being true for most eukaryotic cell samples.

The percent of total RNA, which consists of total mRNA, can vary significantly between different cell types. In bacteria, about four percent of the total RNA consists of total mRNA (10). Since a rapidly growing bacteria cell contains ten times more total RNA than does a slowly growing bacterial cell, the rapid growing cell can contain ten times more total mRNA transcripts than does the slow growing bacteria cell. For mammals, it has been reported that total mRNA transcripts make up from one to five percent of total cellular RNA, depending on the cell type (7, 13). The number of total mRNA transcripts per mammalian cell has been estimated to range from 10⁵ to 10⁶ mRNA transcripts per cell (16). A growing mammalian mouse fibroblast 3T3 cell contains four times more total RNA per cell and six times more total mRNA per cell than does a non-growing 3T3 cell (1, 14). Thus, within a homogeneous population of bacterial or mammalian cells, the total amount of mRNA transcripts per cell can vary 6-10 fold, depending on the cell growth stage.

As discussed earlier, the amount of total RNA per mammalian cell can vary over a range of about twenty-five fold for different cell samples from one mammalian organism (see Table 1). The total RNA content per cell for liver is about twenty-five fold higher than that for thymus. The percent of total RNA values for liver and thymus total mRNA fractions is not known. The actual difference between the total mRNA transcripts per cell amounts for these samples may be very large. If the thymus has 1% total mRNA, and the liver 5%, the difference in total mRNA transcripts per cell would be about 125 fold. Two cell samples, which have one percent total mRNA values, could vary in total mRNA transcript per cell amounts by one to twenty-five fold, depending on the samples compared. Mammalian samples, which have the same total RNA content per cell, may have a five-fold difference in total mRNA transcripts per cell. There is relatively little information available concerning the total mRNA transcript per cell content of different cells and tissue types. The effect of various chemical and physical treatments on these total mRNA transcript per cell values is also not available.

Polyadenylated mRNA (PA⁺ mRNA).

Prior art believes and practices that the vast majority of the total number of mRNA molecules in a eukaryotic cell are associated with a polyadenylate sequence of significant length (7, 8, 13). Such mRNA molecules are termed poly A⁺ mRNA molecules or PA mRNAs. A small number of different mRNA types in a eukaryotic cell are not associated with a PA tail of significant length. These mRNA molecules are termed PA⁻ mRNA molecules, or PA⁻ mRNAs, and they are believed to comprise a very small fraction of the cell's total mRNA molecule population. PA⁻ mRNA is also produced from pre-existing PA mRNA molecules in eukaryotic cells by the specific removal of most of the PA tail from the mRNA. In this context whether a mRNA is PA or PA⁻ is defined by the shortest PA sequence, which will bind to oligo dT (odT) during the PA mRNA isolation step. This is believed to be a PA sequence greater than about 20 nucleotides long.

Prior art believes and practices that the great majority of the total mRNA population of a cell is comprised of PA mRNA molecules which can be isolated and purified by hybridizing with poly dT or poly U sequences. Prior art also believes and practices that the PA mRNA population isolated from a cell sample consists of the great majority of total mRNA molecules in a cell or cell sample. As a consequence of this belief and practice, prior art routinely isolates and analyzes purified PA mRNA fractions from cell samples, and also routinely uses odT priming of total mRNA or isolated mRNA to produce labeled mRNA polynucleotides for microarray and non-microarray gene expression analysis methods RT-PCR and DD-RT-PCR. Prior art also routinely uses purified mRNA for dot blot, northern blot and nuclease protection gene expression analysis. Note that other cell RNA types are not polyadenylated, and these include rRNAs, tRNAs, miRNAs, siRNAs, and snoRNAs.

Gene Expression Analysis.

Gene expression analysis requires the sampling and characterization of a cell sample's population of RNA transcripts. Various gene expression analysis methods are available to produce gene expression profiles for one or more samples (1, 7, 8, 13, 17-26). An expression profile can represent a part, or all, of the RNA transcripts present in a sample. A gene expression profile for the RNA population analyzed should indicate the genes which are detectable as active and those which are not detectable as active, and provide a quantitative measure of the extent, either absolute or relative, of expression for each active gene. The gene expression profiles of two or more sample RNA populations can be compared to identify differences in gene activity and expression extents, which exist between the different samples. A Differential Gene Expression (DGE) profile resulting from the comparison of two different individual gene expression profiles, should indicate whether a gene is expressed as RNA in both cell samples, and should provide a quantitative measure, either absolute or relative, of a gene's number of RNA transcripts per cell which is present in each sample.

These gene expression comparisons are almost always expressed as a differential gene expression ratio, or DGER. The DGER, which actually exists in the intact cell sample or compared cell samples for a particular gene comparison, is termed the true DGER or T-DGER for that particular gene comparison. For a SGDS comparison the T-DGER is equal to the ratio of (the number of particular gene RNA transcripts per cell in one cell sample)÷(the number of the same particular gene RNA transcripts per cell in a different compared cell sample). For a DGDS comparison, the T-DGER is equal to the ratio of, (the number of a particular gene RNA transcripts per cell for one cell sample)÷(the number of a different particular gene RNA transcripts per cell in a different compared cell sample.) For a DGSS comparison the T-DGER is equal to the ratio of (the number of a particular gene RNA transcripts per cell for one cell sample)÷(the number of a different particular gene RNA transcripts in the same cell sample). Note that for the gene expression analysis of one cell sample, or the gene expression analysis comparison of different cell samples, T-DGER ratios exist for each different RNA type in the cell or cells. Such RNA types include but are not limited to rRNA, tRNA, mRNA, siRNA, miRNA, snoRNA, and any other known or unknown RNA type, which is present in the cell.

An aspect of gene expression analysis is the generation of gene activity profiles which are specific for a particular cell sample type such as a cancer cell, a cell treated with a toxic compound, or a cell at a particular stage of differentiation. These gene activity profiles are also termed gene expression signatures or portraits. The gene activity profile for a particular cell is a result of the overall gene activity regulation system which exists in the cell. This system dictates that certain genes are inactive while others are active, and further dictates that some active genes are more active than other active genes. Such a gene activity profile provides information as to which genes are active and provides a quantitative measure as to the extent of activity of each active gene. From such a profile, inferences can be made about which different active genes are expressed together and about the direction of gene regulation forces on one gene relative to one or more different genes. In the same sample, one gene may be measured to be active while a different gene may be measured to be inactive. The prior art inference or interpretation here is that the inactive gene is down regulated relative to the active gene. Similarly, in one cell sample, a particular gene may be detected to be active, while in a compared cell sample the gene is detected to be less active. The common inference here is that the active gene in the one sample is upregulated relative to the active gene in the second sample.

A variety of gene expression approaches and methods can be used to produce gene expression profiles for a cell sample and compared cell samples. It will be useful to divide these methods into two groups. One group includes the microarray methods and non-microarray methods, northern blot methods, dot blot methods, nuclease protection methods, and RT-PCR methods and methods related to these methods of producing gene expression analysis and comparison results such as the well known ELISA, and hydroxyapatite and other affinity column methods. For simplicity, this group is here termed the microarray and non-microarray group. A second group includes the tag or clone counting gene expression analysis and comparison methods, such as the various forms of the tag method, serial analysis of gene expression, or SAGE. Most of the discussion of this communication will involve the microarray and non-microarray methods. Note that the microarray and non-microarray methods and the tag methods can be used for the gene expression analysis of genes other than the mRNA genes. These include the expression analysis of different types of genes for rRNA, tRNA, miRNA, siRNA, snoRNA, and any other known or unknown gene, which is transcribed into RNA.

Microarray and Non-Microarray Gene Expression Analysis.

There is a large literature on the application of microarray and non-microarray and clone counting methods for gene expression analysis of individual cell samples and for gene expression analysis cell sample comparisons (1, 3-9, 12, 13, 16, 22-27). Virtually all of these publications analyze the expression of particular gene mRNA transcripts and report particular gene mRNA transcript quantitative abundance values for a cell sample and/or particular gene mRNA transcript quantitative DGER values for an SGDS cell sample comparison. However, prior art microarray practice believes that it is not possible to measure the absolute mRNA transcript abundance for a particular gene, but that it is possible to accurately and quantitatively measure T-DGER values for SGDS particular gene mRNA transcript comparisons (11, 28, 29). Prior art often uses a non-microarray method to corroborate microarray SGDS comparison measured DGER results for particular genes.

Different formats are used to generate microarray DGER's (30). In the two slides one label format, each sample is analyzed on a separate slide, and the results are compared to generate a DGER for a gene. In this case, two different microarray hybridization solutions must be used. The alternative method is the one slide, two label format, where each sample is labeled with a different label and then mixed together in the same hybridization solution. The results from the different labels are then used to generate a gene's measured and normalized DGER. In this format, only one hybridization solution is used. The following discussion pertains to both these formats, and applies to both mRNA and other types of RNA expression analysis.

The vast majority of prior art microarray and non-microarray gene expression analysis and gene expression comparison analysis practice assays concern the SGDS comparison of the expression of particular gene mRNA transcripts. Very little emphasis has been put on either DGDS or DGSS comparisons of mRNA or any other RNA type, or the expression analysis of RNA types other than mRNA or regulatory RNA. Because of this, the following discussions focus primarily on SGDS comparisons of mRNA transcripts. Nonetheless, the discussions are directly applicable to both DGDS and DGSS mRNA transcript comparisons and SGDS, DGDS, and DGSS comparisons of RNA transcripts of any kind.

Determination of a Microarray or Non-Microarray Assay Measured and Normalized Differential Gene Expression Ratio (N-DGER).

A prior art microarray or non-microarray gene expression analysis assay is almost always designed to measure the relative numbers of the same particular gene mRNA transcripts which are present in different compared cell samples. In other words, the assay is designed to determine the true differential gene expression ratio (T-DGER), for the particular gene mRNA transcripts in the compared cell samples. To accomplish this, prior art compares equal amounts of each cell sample's RNA in the assay. Prior art then believes and practices that for a particular gene which is expressed in each compared cell sample, the ratio in the assay hybridization step or PCR amplification step, of (the number of moles of the particular gene's mRNA transcripts or equivalents, from one cell sample)÷(the number of moles of the same particular gene's mRNA transcripts or equivalents, from the other compared cell sample), is equal to the T-DGER for the particular gene which exists for the compared cell samples. Herein, the hybridization step or amplification step ratio of the cell sample compared particular gene mRNA or RNA transcripts or equivalents, is termed the assay concentration ratio, or ACR. Prior art then, generally believes and practices that for a gene expression comparison analysis assay, (ACR)=(T-DGER) for the RNA transcripts being compared.

For a microarray or non-microarray gene expression assay, a measured particular gene expression extent comparison for compared cell samples is almost always reported in the form of a normalized DGER value. Herein, a normalized DGER is termed a N-DGER for a gene expression comparison. A N-DGER value for a particular gene comparison is believed by the prior art to accurately reflect the ACR value for a particular gene comparison in the assay hybridization step, or PCR amplification step. Prior art then believes that for a gene expression comparison analysis assay, (N-DGER)=(ACR)=(T-DGER), for a particular gene comparison.

A prior art microarray N-DGER for a particular gene comparison is derived from the assay measured quantitative signal activity associated with each cell sample's mRNA or equivalents, which has hybridized to the particular gene's microarray spot. In order to generate the gene's assay measured N-DGER value, total signal activity associated with the spot is measured. Herein this total spot signal is termed the TSS. Before normalization the prior art almost always adjusts each TSS for assay background signal and imaging associated factors by subtracting the appropriate background signal value from each particular gene TSS value, thereby producing a raw assay signal value for each compared particular gene. Herein the raw assay signal is termed the RAS, while the gene comparison's RAS ratio is termed the RASR. The RAS value for a cell sample's gene is believed to represent only signal which is associated with labeled mRNA polynucleotide molecules which are immobilized to the spot by hybridization. Prior art generally believes that the assay RAS or RASR result for each gene must be adjusted, corrected, or normalized, before biologically meaningful interpretations of the assay signal or N-DGER results can be made (31). Herein, a gene's normalized RAS is termed a normalized assay signal, or NAS, while the gene comparison NAS ratio is termed the NASR. A gene comparison's NASR is equal to the ratio of, (the gene's NAS for one cell sample)÷(the same gene's NAS for another cell sample). Note that as discussed, by definition the (assay NASR)=(assay N-DGER) for a particular gene comparison. Prior art microarray and non-microarray practice believes that when an assay RASR value for a particular gene comparison is normalized for prior art considered assay variables, the resulting NASR value accurately reflects the ACR value for the particular gene which is associated with the hybridization step, or the PCR amplification step, of the assay. Prior art then, believes and practices that (NASR=ACR) for the particular gene comparison. Further, because prior art believes that (ACR=T-DGER) for the particular gene comparison, then prior art also believes and practices that (NASR=ACR=T-DGER) for a particular gene comparison. Overall then, prior art believes and practices for a particular gene comparison that, (N-DGER=NASR=ACR=T-DGER).

A prior art non-microarray northern blot, dot blot, or nuclease protection, assay produced N-DGER value for a particular gene comparison is derived from the assay measured quantitative signal activity associated with each cell samples mRNA. In order to generate the particular genes assay measured N-DGER value, the TSS associated with each cell sample RNA is measured, and then corrected for background to produce a particular gene RAS value for each cell sample RNA, and a particular gene RASR value for the particular gene comparison. The RASR value is then normalized to determine the assay measured particular gene NASR and N-DGER value. Prior art non-microarray practice believes that in the assay the particular gene (NASR=N-DGER=ACR).

A prior art non-microarray RT-PCR assay produced N-DGER value for a particular gene comparison, is derived from assay measured absolute or relative values for the number of particular gene cDNA molecules which are present in the assay PCR amplification step at time zero for each compared cell sample. The actual ratio in the assay per amplification step at time zero, of the number of particular gene cDNA molecules compared is equivalent to the assay ACR value. The prior art assay measured ratio of these compared cell sample particular gene cDNA molecule numbers is equal to the particular gene comparison RASR assay value. Upon normalization, the prior art RASR value equals the particular gene NASR value, which by definition equals the measured N-DGER value. Prior art RT-PCR practice then, believes that in the assay, the particular gene (NASR=N-DGER ACR). Note again that this discussion applies directly to gene expression analyzes for different types of rRNAs, tRNAs, siRNAs, miRNAs, snoRNAs, and any other known or unknown RNA in a cell.

Normalization of microarray and non-microarray and clone counting method gene expression assay results, is necessary because of the existence of assay variables which influence the assay value of the RASR, but are related to variables in the assay materials, assay process, assay design, or assay signal measurements, and are not related to the relevant biological difference in gene expression which exists between the assay compared genes. Prior art has identified a variety of such assay variables and a large literature exists concerning prior art normalization approaches for prior art known assay variables (7, 8, 28-72). These prior art variables are discussed in the next section.

Microarray and Non-Microarray Gene Comparison Assay Variables.

Normalization of a particular gene comparison assay RASR result involves adjusting or correcting the particular gene RAS or RASR result of interest for the effects of assay variables which are pertinent to the particular gene comparison assay. Such normalization is accomplished for a particular pertinent assay variable by adjusting the particular gene comparison assay RASR value with the quantitative value of an assay normalization factor which corrects the assay RASR for the effect of the particular assay variable. Herein the assay normalization factor for a particular assay variable is termed a normalization factor, or NF. All particular assay variable NF values can be expressed in terms of the effect of the NF value associated with one cell sample on the deviation of the particular gene RAS value from accuracy. The effect of the compared cell sample's particular assay variable NF values on a particular gene RNA transcript comparison RASR value is expressed in terms of the ratio of the particular assay variable NF value associated with each compared particular gene RNA transcript comparison herein, this ratio is termed the NF ratio, or more practically, just the NF. Prior art expresses particular NFs in terms of ratios and also in non-ratio terms. Herein, NFs will refer to both.

For a particular gene comparison assay RASR value, when the assay value for a pertinent assay variable NF ratio is equal to one, the assay value for the assay RASR does not require normalization for the particular assay variable. However, when the assay value for a particular assay variable NF is not equal to one, the particular gene comparison assay RASR value will require normalization for the particular NF, unless the NF≠1 assay value is compensated for by a different particular assay variable NF value. As will be discussed later, if a particular gene comparison assay RASR value is properly normalized for all assay pertinent NFs, then the resulting particular gene assay NASR value is equal to the gene's T-DGER which is present in the assay. However, if the particular gene comparison assay RASR is not normalized for all assay pertinent NFs, or is normalized with an incorrect NF assay value, the resulting gene assay NASR value will not equal the gene's T-DGER. This indicates the necessity to first identify the pertinent NFs for each particular gene comparison, and then to directly or indirectly obtain an accurate measure of the assay value for each particular NF, and then to normalize the particular gene RASR value, either directly or indirectly, for each pertinent assay variable NF. If all of the pertinent NFs can be correctly normalized for, then the resulting (NASR=T-DGER) for the particular gene comparison. Herein an assay pertinent NF is an NF which is associated with assay variables which can cause an assay measured particular gene RAS or RASR value to be inaccurate. For a particular gene RNA transcript comparison assay, when the pertinent NF ratio for a particular gene RNA transcript comparison is equal to one, the NF can be ignored for normalization.

Assay variables include both global variables and non-global variables. A global variable NF has an equal effect on each particular gene expression assay RASR result in the cell sample comparison assay. For a cell sample comparison assay, there is only one quantitative assay value for a particular global NF, and that same NF value is applied to each particular gene comparison RASR in the assay. There can be more than one pertinent global variable in each cell comparison assay, and each different global NF can have a different quantitative value. A non-global assay variable often does not have the same effect on each particular gene comparison in the cell comparison assay. For one cell comparison assay there may be multiple different quantitative values associated with a single non-global variable NF, and a particular non-global NF value may be pertinent to a particular subset of gene comparisons in the assay, while a different NF value for the same non-global assay variable NF may be pertinent to a different subset of one or more gene comparisons in the same cell comparison assay. For each pertinent non-global NF value it is necessary to be able to directly or indirectly measure, or otherwise determine, the assay value or values for each particular assay pertinent non-global NF, and to identify the gene comparison subset associated with each particular different assay value for the particular non-global NF. There can be, and almost always are, multiple different types of pertinent non-global variables associated with a typical microarray or non-microarray cell comparison assay.

As an example, microarray prior art practice has identified and often considered during the normalization process, five different non-global assay variable NFs which are often observed in a cell sample comparison assay. These are the spatial, print tip, print plate, intensity and scale assay variables (7). Each of these different non-global variables is associated with multiple NF values, each of which applies to a different subset of compared genes. Prior art methods which claim to be able to identify the gene comparisons in a microarray assay which are associated with a particular pertinent assay variable, and to determine the particular pertinent assay variable NF value necessary for correct normalization, have been reported for each of the spatial, print tip, print plate, intensity, and scale, non-global assay variables. Each of these reported methods requires one or more prior art assumptions to be valid in order to correctly normalize. Note that prior art microarray practice seldom, if ever, independently determines the assay NF values associated with the above discussed prior art considered global and non-global assay variables. Instead the prior art normalization process often relies on certain assumptions which allows for the normalization of these considered global and non-global assay variables, without having to experimentally determine the assay variable NF values. If these prior art assumptions are not valid, then the prior art normalization of these prior art considered variables is not valid.

In a prior art microarray cell sample comparison, each particular gene assay derived RASR value is almost always associated with one or more global assay variables, and one or more non-global assay variables, and each of the particular non-global assay variables, and each of the particular non-global assay variables is almost always associated with multiple different NF values, each of which applies to a different subset of compared genes. For any particular gene comparison, the aggregate effect of these pertinent global and non-global NF values causes the assay measured RASR value to deviate from the biologically accurate T-DGER for the gene comparison in the cell comparison. In such a situation, the separate NF values for each pertinent global or non-global assay variable can interact in a way to cause the deviation to be small, or large, or non-existent. In order to correctly normalize the assay measured RASR value for each particular gene comparison in the assay, it is necessary to somehow obtain an accurate value for the aggregate effect of the global and non-global assay variable NFs which are pertinent to the particular gene comparison. It is generally unlikely that this can occur unless the pertinent assay variables can be identified, and the method for obtaining the NF values for those pertinent variables is valid. Prior art microarray SGDS particular gene mRNA transcript comparison practice, almost always relies on the assumptions that most genes in the cell sample comparison are unregulated, and/or that such unregulated genes can be known or identified, in order to determine and normalize for both the global and non-global NFs. If these assumptions are not correct, the prior art normalized results cannot be known to be correct.

Prior art microarray and non-microarray gene expression analysis practice has reportedly normalized for a variety of particular global and non-global assay variables (7, 35, 41, 62). These include but are not limited to, assay variables related to the following assay factors.
(a) The efficiency of labeling and detection of the mRNA derived labeled polynucleotide molecules representing each compared cell sample. Herein, the mRNA derived labeled polynucleotide molecules are termed mRNA LPN molecules or LPN molecules. A prior art known and considered NF which is associated with the efficiency of labeling and detecting a cell samples total mRNA LPN preparation is the total mRNA signal activity ratio for the compared cell samples, herein termed the assay TSAR. The prior art regards the TSAR as a global NF.
(b) Deviations away from comparing in the assay, equal masses of total RNA or mRNA or equivalents from each cell sample. The prior art known and considered NF which is associated with the amount of each compared cell sample's RNA compared in the assay, is herein termed the added RNA ratio or ARR (18, 83, 96). The ARR is a global NF.
(c) Differences in the assay hybridization conditions on the assay hybridization kinetics of the compared cell sample mRNA LPNs. The prior art known and considered NF, which is associated with such hybridization kinetic differences, is herein termed, the assay hybridization condition hybridization kinetic ratio, or C-HKR. The C-HKR is generally a global assay variable.
(d) Variations in the signal activity of gene comparison results which correlate with particular areas of the microarray device. The prior art known and considered NFs, which are associated with these location specific signal activity differences, is herein termed the spatial or surface NFs. This location related NF is a non-global NF.
(e) Variations in the signal activity of assay gene comparison results associated with the overall signal intensity present in the spot. The prior art known and considered NF, which is associated with this effect, is a non-global NF, and is herein termed the intensity NF. The intensity NF is a non-global NF.
(f) Differences in the microarray assay signal activity of assay gene comparison results which correlate to one or another aspect of the microarray spot printing process. The prior art known and considered NF, which is associated with printing process aspects, is herein termed the print process or print tip NF. The print process NF is a non-global NF.
(g) Differences in microarray assay signal activity of assay gene comparison results, which correlate to certain variations in the different microwell plates, which are used to produce a microarray device. The prior art known and considered NF, which is associated with the print plate, is herein termed the print plate NF. The print plate NF is a non-global NF.
(h) Variations in the microarray signal activity of assay gene comparison results which correlate to one or another aspects of the image analysis process used to obtain the spot signal activity results. The prior art known and considered NF, which is associated with the image analysis process, is herein termed the image analysis NF. The image analysis NF is a non-global NF.
(i) Variations in the signal activity of assay gene comparison results, which correlate with the various aspects of random noise, associated with the assay. The prior art known and considered NF, which is associated with the random noise, is herein termed the random noise NF. The random noise NF is a non-global NF.
(j) Variations in the microarray assay background signal activity, which is associated with different gene comparison signal activity results. The prior art known and considered NF, which is associated with assay background, is herein termed the background NF. The assay background NF is a non-global NF. Here, variations in assay gene comparison signal activity results, which are related to the non-specific association of the particular gene LPNs with the microarray spot and surface, are considered to be part of the background signal.
(k) Differences in compared cell sample cDNA or cRNA synthesis efficiencies, and between cell sample and assay standard cDNA synthesis efficiencies, for microarray and RT-PCR assays. The common existence of such differences in synthesis efficiency is known to the prior art (7, 13, 97-114). However, such differences are only rarely determined and considered during normalization by the prior art. These cDNA synthesis differences are associated with non-global assay variables. Here such a cDNA synthesis efficiency is termed a cell sample cDNA synthesis yield. Such a cDNA synthesis yield is measured in terms of the fraction of the template RNA which is converted to cDNA, and this is termed the cDNA synthesis YF or cDNA YF.
(l) Differences in RT-PCR assay cDNA amplicon equivalent amplification efficiencies associated with the PCR amplification step between: compared cell sample particular gene cDNAs; compared internal or external standard cDNAs or DNAs; a particular gene cDNA and the internal or external standard DNA or cDNA associated with it. Herein, such a cDNA or DNA amplicon equivalent amplification efficiency is termed a cDNA AE•AE. The AE•AE value is greatly influenced by the PCR E value for the particular gene or standard cDNA or DNA, and it is commonly known that such E values and AE•AE values often vary very significantly for compared cell sample particular gene and standard cDNAs (104, 106). However, such differences are only rarely determined and normalized for. Both the cell sample particular gene and internal and external standard cDNA AE•AE values are associated with non-global assay variables.

Note that a designated particular assay variable may represent multiple related sub-variables, and a quantitative assay NF value for such a particular variable category will take into consideration each of the related sub-variables. As an example, the TSAR normalization factor value includes contributions from both the efficiency of labeling, and the efficiency of label detection sub-variables. In addition, a particular assay measured NF value may incorporate one or more of the above listed assay variables into one quantitative NF value. Each of the noted assay variable types is not pertinent for every microarray or non-microarray gene expression assay. Different gene expression analysis methods and designs require the consideration of different assay variables and NFs. In addition, gene expression analyzes of different RNA types such as mRNA, rRNA, tRNA, miRNA, siRNA, snoRNA, and any other known or unknown RNA in the cell can be associated with different assay variables.

Other known potential sources of assay variability are generally not taken into consideration in prior art normalization practice. These include but are not limited to, the following. (i) Variability associated with the degradation of analyzed cell sample nucleic acids or the nucleic acids derived therefrom. (ii) Variability associated with differences in the representation and frequency of occurrence of each particular mRNA in a cell sample isolated total RNA or mRNA, or nucleic acids derived therefrom, relative to the representation and frequency of occurrence of each mRNA in the intact cells of a cell sample. (iii) Variability associated with differences in the efficiencies of transcription of RNA into cDNA and cRNA. (iv) Variability associated with differences in the efficiencies of isolation and purification of cell sample total RNA and mRNA and nucleic acids derived therefrom. (v) Variability associated with the effect of the nucleotide length of the analyzed nucleic acid molecules on the assay hybridization kinetics, and on assay signal activity associated with particular mRNA LPNs in the assay. (vi) Variability associated with the effect of the nucleotide sequence of the analyzed nucleic acid molecules on the assay hybridization kinetics, and on the assay signal activity associated with particular mRNA LPNs in the assay. (vii) Variables associated with the effect of the assay signal activities of a particular gene's compared mRNA LPN molecules on the assay gene comparison signal activity result. (viii) Variables associated with the effect of the direct or indirect signal label associated with the compared mRNA LPN molecules, on the assay hybridization kinetics of the cell sample mRNA LPN molecules, and the assay stability of the cell sample hybridized LPN molecule duplexes. (ix) Variability associated with attaching signal generation complex molecules to hybridization immobilized indirectly labeled LPN ligands. (x) Variability associated with second strand cDNA synthesis during the first strand cDNA synthesis step. (xi) Variability associated with the synthesis of unwanted non-target cRNA during the cRNA synthesis step. (xii) Variability associated with the erroneous quantitation of the input RNA or cDNA or cRNA for a gene expression assay. (xiii) Variability associated the commonly occurring non-linear relationship between the observed assay signal and the amount of input sample RNA or cDNA or cRNA for the assay (66, 70, 71). The above described potential sources of variation for microarray and non-microarray assays are generally not determined and considered in the prior art microarray and non-microarray normalization process. Since prior art generally believes and practices that a prior art measure particular gene comparison normalized NASR value is biologically accurate, the prior art must believe that the above described potential sources of assay variability are insignificant. Alternatively, the prior art does not know about them.

Replicates within an assay provide information on various sources of variability, which occurs in a microarray or non-microarray assay. Appropriately positioned replicate microarray spots for one or more expressed and non-expressed cell sample genes are routinely incorporated into the microarray assay in an attempt to determine the quantitative values for assay NFs (7). Also incorporated are appropriately positioned replicate spots for one or more standard RNA or DNA sequence which is not naturally present in the compared cell samples nucleic acids, but is added to each compared cell sample in order to determine quantitative assay NF values. Such added nucleic acids or nucleic acids derived therefrom are herein termed exogenous standard molecules or exogenous S molecules.

A variety of different approaches have been utilized for the prior art normalization of microarray and non-microarray gene expression analysis results (7, 8, 28-72). There is no standard method of normalizing such results. Different prior art microarray and non-microarray assay practitioners make different normalization assumptions, determine and consider for normalization different assay variable associated NFs, and utilize a variety of different statistical methods for normalization. As an example a particular assay variable NF may be associated with non-linear effects, and prior art statistical methods provide a means for normalizing for both linear and non-linear NF effects.

In addition there is no standard microarray or non-microarray assay design, and different assay designs are often associated with different assay pertinent assay variable associated NFs. Even in the same microarray or non-microarray assay different pertinent assay variable NF combinations are associated with SGDS, DGDS, and DGSS comparison assay measured particular gene RASR values. Further, different particular gene spots in the same assay can be associated with different pertinent assay variable combinations.

Assumptions Required for Prior Art Normalization.

There are numerous prior art approaches for normalizing microarray assay measured particular gene mRNA transcript SGDS comparison assay results (7, 8, 28-72). Each method requires one or more essential assumptions which must be true in order for the normalization process prior to give biologically relevant and accurate results (7, 34, 35, 41, 43, 45, 46, 48, 51, 52, 53, 62, 136, 137, 138). Prior art known assay variables which have been considered by the prior art to be significant enough to be utilized and considered for prior art normalization of microarray and non-microarray gene comparison results, are described in the previous section on assay variables. Few of the prior art normalization methods correct for all of the described known and considered assay variables or NFs. This makes it likely that many of the prior art normalized gene comparison assay results, are incompletely normalized for prior art known and considered assay variables.

Prior art believes that for a particular gene comparison, the prior art normalization of the gene's assay RASR value produces an assay NASR value which is equal to the gene's T-DGER in the compared cell samples. Since, by definition, the (assay NASR)=(assay N-DGER), the prior art believes that the (assay NASR)=(assay N-DGER)=(T-DGER), for the gene. In order for this to be true, the prior art believes or assumes that the (assay N-DGER)=(assay NASR)=(ACR)=(T-DGER), for the gene.

All prior art normalization approaches must make one or more assumptions in order to derive quantitative values for assay variable normalization factors. All or essentially all prior art normalization approaches assume one or more of the following assumptions in order to derive normalization factors for normalizing the gene comparison assay results. (i) Most of the genes which are active in both compared cell samples are unregulated (7, 51). (ii) For those genes which are regulated in the cell sample comparison, there is a balance between the up and down regulated genes (7). (iii) In a cell sample comparison enough unregulated genes can be identified so that the identified unregulated genes can be used as internal reference genes from which normalization factors (NFs) can be derived, and these NFs can be used to normalize other genes in the cell sample comparison (7). (iv) The spotted genes on the array represent a significantly large random selection of the compared cell sample genes (7). (v) The total RNA content per cell is the same for each compared cell sample (52, 138). (vi) The total mRNA content per cell is the same for each compared cell sample (46). (vii) One or more genes which are a priori known to be active in both compared cell samples, are known to be unregulated or known to be regulated to a particular quantitative extent, and such genes serve as internal references from which NFs can be derived, and these NFs can then be used to normalize the other genes in the cell sample comparison. Such known genes have been termed housekeeping genes (7, 31, 50). Note that for a particular prior art normalization approach, the assumptions required to implement that normalization approach must be valid in order for the normalization process to be valid, and in order for a particular gene comparison result to be normalized correctly. Note further that a particular gene comparison normalized result may be correctly normalized for the assay variables which are considered in the normalization process, but not completely normalized for all pertinent assay variables.

Perhaps the most widely used normalization approach is the global normalization method of total intensity normalization or TIN, which is also called global mean normalization, or global median normalization (7, 31, 50). This approach assumes the above described assumptions (i), (ii), (iv), and some investigators believe that assumption (v) and (vi) must also be made. Assumptions (i) and (ii) have not been experimentally confirmed and are necessary in order for the TIN normalization to be valid. Prior art acknowledges that assumption (iv) is not valid for low density microarray applications and that it is inappropriate to use the TIN method in such a situation. Prior art believes that with these assumptions the summed assay signal intensity values associated with each cell sample will be approximately the same, and when they differ, the difference is due to differences in the amount of added cell sample mRNA or equivalents, and/or LPN labeling efficiency and/or detection. When the summed total assay signal intensities from each cell sample differ an assay global NF can be determined. This global NF value is then used to normalize the RASR for each particular gene comparison in the assay. The global TIN or global mean method of normalization cannot be used to normalize for non-global assay variables such as intensity differences due to spatial or local differences in signal intensity, nor does it correct for intensity dependent signal biases, or biases associated with the array print tip differences. Such biases are non-global biases, and can be corrected for using a variation of the TIN method, the local mean normalization method (7, 31, 50). For this method the same three assumptions are necessary for valid normalization. Note that for the TIN, and variations of the TIN method, while it is necessary to assume that most of the genes in the comparison are unregulated, it is not necessary to know which genes are unregulated.

The other widely used prior art normalization approach does require being able to identify unregulated genes in the assay results from the gene comparison assay (7, 31, 50). This can be done in a variety of ways including scatterplot, linear, and non-linear regression analysis, and ranking methods. This approach assumes the above described assumption (i), (iii), and (iv), and some investigators believe that assumptions (v) and (vi) must be made. Assumptions (i), (iii) and (vi), have not been experimentally confirmed, and are necessary in order for the normalization approach to be valid. This approach is most often used to obtain a global NF, which is then applied equally to all gene comparisons in the assay. However, the global NF cannot be used to normalize for the prior art considered spatial, intensity, or pin tip, non-global assay variables, or any other non-global assay variables. Such non-global assay biases can be normalized for by using variations of this approach which use loess regression analysis or ranking methods (7, 31, 50, 69). For these methods the same assumptions (i), (iii) and (iv) are required for the normalization.

Another widely used prior art normalization method utilizes above described assumption (vii), where it is assumed that the identity of one or more genes, which are unregulated, is known a priori (7, 31, 50). This approach is widely viewed in the prior art as inappropriate, and there is significant experimental evidence that such an assumption is not often valid. There is little or no valid experimental evidence that the housekeeping gene approach has any validity.

An additional widely used prior art normalization method involves the incorporation of one or more exogenously added control mRNAs into the assay. Such controls can be useful for normalizing assay biases related to mRNA LPN labeling and detection, the quantity of RNA or mRNA added to the assay, the signal intensity, spatial biases, and various hybridization biases. Here the above mentioned assumptions do not apply. The prior art use of these control molecules does not address the biases associated with the intrinsic biologic aspects of the assay, and therefore are not adequate for the complete normalization of the gene comparison results.

Except for the method involving the exogenous addition of control mRNA, prior art believes and practices that the above described prior art normalization approaches result in the conversion of particular gene comparison assay RASR values to assay NASR values which are equal to the T-DGER of the particular gene comparison in the compared cell samples. Prior art indicates that such normalization adjusts for global assay biases related to differences in the amounts of cell sample RNA added to the assay, differences in the labeling efficiencies and detection of cell sample mRNA LPNs, and any differences in the hybridization kinetics of the cell sample LPN related to the assay hybridization conditions. Prior art has also adapted these normalization approaches for normalizing for the non-global assay biases related to spatial, intensity, and print tip assay variables.

Virtually all, or all, prior art microarray and non-microarray gene expression comparison analysis assay normalization, concerns the normalization of the SGDS mRNA transcript comparison assay results.

Many non-microarray gene expression analysis RASR results are also normalized. This is often done for northern blot and dot blot assays by including in the assay an externally added internal control or loading control in order to detect deviations from the assay comparison of equal masses of compared cell sample RNAs (18, 96). This internal control allows the determination of a quantitative NF value, which corrects for the amount of each cell samples RNA added. Added internal control molecules are also utilized for normalization of the various methods, which use RT-PCR for gene expression analysis. Housekeeping genes are also used for these purposes (74, 75, 77).

Interpretation of Positive and Negative Gene Activity Results.

A variety of gene expression measurement methods have been used to compare cell samples in order to identify genes which are expressed, i.e., active, in both samples, and genes which are active in one sample, and not the other. These include microarray and non-microarray methods such as northern blotting, dot blotting, nuclease protection, RT-PCR and different versions of differential display methods. In such a comparison, a positive result for a particular gene can be interpreted with certainty. It means that the amount of the sample's total RNA, total mRNA, or LPN equivalents (such as cDNA or cRNA), which was added to the assay contained a detectable amount of that particular gene's mRNA transcripts, and therefore it can be concluded that the gene is active in the sample. For microarray assays, the amount of total RNA, total mRNA, or equivalents, added to the assay refers to the amount added to the microarray hybridization solution. For northern blot assays, it is the amount loaded in the electrophoresis gel. For nuclease protection assays, it is the amount of RNA hybridized to the labeled probe. For dot blot assays it is the amount loaded on the filter. For RT-PCR assays, it is the amount added to the RT-PCR amplification solution. For differential display methods, it is the amount of sample mRNA used to make the cDNA.

In the event a negative result is obtained for the particular gene in a second sample, the interpretation is less certain. What is certain is that the amount of the second samples total RNA, total mRNA, or equivalents, which was added to the assay contained an undetectable amount of the genes mRNA transcripts. However, the presence of a finite, but undetectable, amount of the gene's mRNA transcripts in the added second sample RNA, or equivalents, cannot be ruled out. In other words, the negative result may be a false negative result. A false negative will occur when the gene is active in the sample, but not active enough for a detectable amount of the gene's mRNA transcript to be present in the amount of sample RNA, or equivalents, added to the assay. Thus, when a negative result is obtained, it is not known whether the result is a true negative, or a false negative. In the case of a true negative situation, the gene is not expressed in the sample, and adding a greater amount of sample RNA, or equivalents, cannot change the negative result. In the false negative situation, adding a greater amount of sample RNA can result in adding a detectable amount of the gene's mRNA transcripts, or equivalents, to the assay. A positive result will then be obtained, and the gene will be considered to be active in the sample. Thus, a change in the amount of sample RNA, or equivalents, added to the assay can result in converting a true positive (the gene is active in the sample), to a false negative result, or converting a false negative result to a true positive result. Such conversions could occur with as little as a two fold or less, change in the amount of sample RNA added. Clearly the decision concerning the absolute amount of each compared samples total RNA, total mRNA, or equivalents, to add to the assay is a very important one, and has a great effect on the interpretation, and utility, of gene activity results.

Prior art believes that a microarray or non-microarray gene expression analysis assay N-DGER and NASR for a particular gene comparison, directly reflects the ratio in the assay hybridization solution of, (the quantitative molar concentration of the particular gene's mRNA transcripts, or equivalents, from one cell sample)÷(the quantitative molar concentration of the gene's mRNA transcripts, or equivalents, from the other cell sample). This ratio is herein termed the assay concentration ratio, or ACR, for the particular gene comparison. Prior art believes then that for a particular gene comparison, (N-DGER)=(NASR)=(ACR). The N-DGER for a particular gene then, depends on the mass of each cell sample's total RNA or mRNA, or equivalents which the investigator adds to the assay hybridization solution, or in the case of RT-PCR the assay PCR amplification solution. A specific amount of added cell mRNA or equivalents from one cell sample will contain an unknown number of mRNA transcripts. Similarly, a specific amount of added cell mRNA or equivalents from the other compared cell sample will also contain an unknown number of the gene's mRNA transcripts, or equivalents. Prior art believes that the ratio in a hybridization solution of, (the added number of the gene's mRNA transcript molecules from one sample)÷(the added number of the same gene's mRNA transcript molecules from the other sample), determines the N-DGER for the gene in a microarray. It is obvious that if the ratio of added sample total RNA, or mRNA, or equivalents is changed, then the ratio of (the added number of genes mRNA transcript molecules from one sample)÷(the added number of the genes mRNA transcript molecules from the other sample), will also change, and the N-DGER for the gene will change. The N-DGER will change in direct proportion to the change in the added sample ratio. Thus, two different N-DGER values for the same sample comparison can be obtained by simply changing the added amount of one, or the other, or both, sample total RNA's, mRNAs, or equivalents. If the sample added ratio changes by a factor of ten, then the N-DGER also will change by tenfold. Clearly, the decision as to the amount of each samples total RNA, mRNA, or equivalents, to add to the hybridization solution is an important one.

The above discussion is also applicable to non-microarray gene expression analysis nuclease protection, RT-PCR, and the various differential display methods. As above, the decision as to the amount of each samples total RNA, total mRNA, or equivalents, to directly compare in an assay is an important one. A discussion of how the current microarray and non-microarray practice addresses this decision follows.

Note again that the above and following discussion is directly applicable to the gene expression analysis of different types of, rRNA, tRNA, miRNA, siRNA, snoRNA, and any other known or unknown RNA type in the cell, as well as DGDS and DGSS gene expression analysis comparisons.

Current Method For Determining the Relative Amounts of Cell Sample Nucleic Acid Compared in the Assay.

In current microarray and non-microarray gene expression comparison practice, the relative amount of each cell samples T-RNA or mRNA or other RNA transcript which is used in the assay comparison, is determined by the “equal amount compared” rule, or the EA Rule. The EA Rule specifies that equal mass amounts of each cell sample total RNA or mRNA be compared in the assay. Essentially all microarray or non-microarray gene expression analysis practitioners follow, or attempt to follow, the EA Rule.

Also common to the non-microarray and microarray methods is the use of an internal control in the assay. This control consists of one or more genes' mRNA transcripts which are naturally present in the RNA's of all samples compared. This control is termed a loading control, or housekeeping gene control (18, 96). Such a control is considered to be necessary in both microarray, and non-microarray methods, in order to control for experimental variables which are unrelated to the differences in gene expression. These include those variables, which can cause deviations from the ideal practice of the EA Rule. When a difference in mRNA transcript levels is detected, the interpretability of the result depends on whether, and to what extent, the detected difference is due to real transcription differences for the gene, or to some other factor. Under certain conditions a housekeeping gene mRNA can be used to determine this.

A key requirement for the valid use of a gene's mRNA as an internal control is that the level of this gene's expression must be the same in all compared samples. In this context, the level of expression of mRNA transcripts in a sample refers to the fraction of the total RNA or total mRNA, which consists of the internal control mRNA transcripts. Thus, the resulting control gene signal in a microarray, assay, or non-microarray assay, is proportional to the total amount of a sample's total RNA or total mRNA being examined (139). An internal control housekeeping mRNA is intended to indicate the relative amounts of each sample's total RNA, or total mRNA, which are being compared in the assay. In other words, the control is intended to control for deviations from the ideal practice of the EA Rule. If, in fact, equal amounts of each sample RNA's are not being compared in the assay, the control mRNA provides a means for correction. The mRNA's of various housekeeping genes have been used as internal controls for both microarray and non-microarray assays. Thus far these controls have had limited usefulness. The current belief is that there are no housekeeping gene mRNA's which are present to the same extent in all samples, which could be compared (109). This is true even for different cell sample types from one mammalian organism. However, for a comparison of particular samples it has been reported that particular housekeeping mRNA's are expressed at similar levels in these cell samples, and can therefore be used as valid internal controls (109).

Current Method For Determining the Relative Amounts of Cell Sample cDNA or cRNA Compared in the Assay.

Only rarely is cell sample total RNA or mRNA compared in prior art microarray or non-microarray gene expression comparison assays. Generally for these assays a cell sample mRNA equivalent, such as cDNA or cRNA, which is produced from the cell sample T-RNA or mRNA, is compared in the assay. For the non-microarray gene expression comparison assays such as northern blot, dot blot, and nuclease protection assays, the cell sample T-RNA or mRNA is compared directly in the assay.

For microarray and RT-PCR related gene expression comparison assays, the cDNA and cRNA are produced from the compared cell sample T-RNA or mRNA by standard methods (7, 8, 116). For such a cell sample cDNA or cRNA comparison, equal amounts of T-RNA or mRNA from each compared cell sample is virtually always used to produce the cDNA or cRNA for the cell sample gene expression comparison. Thus the EA Rule is practiced for the assay, in that an equal amount of T-RNA or mRNA from each cell sample is compared in the assay. Here however, cell sample RNAs are not compared directly in the assay, but indirectly compared in the assay through the cDNA or cRNA mRNA equivalents. For both the microarray and RT-PCR related assays, the mRNA equivalent, not the mRNA, is directly compared in the assay. This is done for the microarray assays by incorporating the cDNA or cRNA into the assay hybridization solution. This is done for the RT-PCR related assays by incorporating the cDNA into the PCR reaction mixture.

For most prior art microarray comparisons of cell sample cDNA preps, the amount of each cell sample's cDNA which is directly compared in the assay, is the amount of cDNA produced from the cell sample T-RNA or mRNA. For other microarray, and RT-PCR related cDNA comparisons, an equal proportion or amount of each cell sample cDNA prep is compared in the assay. It is known that the cDNA synthesis efficiency yield fraction (YF), that is the amount of cDNA produced from a given amount of T-RNA or mRNA, is rarely equal to one, and can be affected by a variety of assay factors (7, 13, 97-114). These include the source of the RNA, the amount of template RNA present, the integrity of the RNA, the enzyme used, the primer type used, and label effects. It is known that the purity and integrity of T-RNA and mRNA from different sources can vary significantly for different RNA preparations. It is also common practice to compare cDNAs associated with different labels. Prior art cell sample cDNA prep synthesis yield fraction efficiency is almost always significantly less than 1, and commonly ranges from roughly 0.1 to 0.5 for oligo dT and specific gene primed cDNA and the synthesized cDNA is almost always significantly shorter in nucleotide length than the template RNA which produced the cDNA. The cDNA synthesis efficiency for random primed cDNA is often higher than that of oligo dT primed cDNA. This indicates that: (i) The amount of cDNA produced for a cell sample mRNA is almost always significantly less than the amount of mRNA template present in the cDNA synthesis mixture; and (ii) the amount of cDNA produced for a given amount of one compared cell sample T-RNA or mRNA, can be significantly different than the amount of cDNA produced for the same amount of T-RNA or mRNA from the other compared cell sample. Because of all this, and because prior art seldom determines the quality or quantity of cDNA produced from each cell samples T-RNA or mRNA, neither the absolute nor the relative amounts of compared cell sample cDNAs are known, or can be known, for the vast majority of prior art microarray, or RT-PCR related, gene expression comparison assays. In addition, the compared cDNAs are often different in average nucleotide length.

For each compared cell sample cDNA prep, prior art believes and practices that the representation and frequency of each particular gene mRNA transcript cDNA equivalent in the cell sample cDNA prep, is the same as the representation and frequency of the particular gene mRNA transcript in both the cell sample RNA prep used to produce the cell sample cDNA prep, and in the intact sample cell. This belief or assumption must be valid, or nearly valid, in order to obtain biologically correct particular gene expression comparison results which are interpretable.

cRNA is the RNA equivalent of cDNA, and is produced from cDNA by standard procedures. For the production of a cell sample cRNA prep from a cell sample T-RNA or mRNA prep, single strand cDNA is first produced from the RNA, using a special primer. Then the cell sample single strand cDNA is converted to double strand cDNA. Because of the special primer, each of the double strand cDNA molecules is associated with a promoter, which allows multiple cRNA molecules to be produced from each double strand cDNA molecule. This results in a manyfold amplification of the cRNA, relative to its template DNA molecule. Such a cell sample cRNA prep can be labeled during synthesis, purified, and compared to another cell sample cRNA labeled prep in a microarray gene comparison assay. Alternatively, a cell sample unlabeled cRNA prep can be further amplified by using a special primer to convert the cRNA to first strand cDNA, then double strand cDNA, and then even more cell sample cRNA. Multiple such amplification cycles can be done for a cell sample cRNA if desired.

For a cell sample cRNA comparison, equal amounts of each compared cell sample's isolated T-RNA are almost always used to produce the first strand cDNA prep for each cell sample, and each cDNA prep is then used to produce a cell sample cRNA prep for comparison. For this process, only rarely is the amount of first strand cDNA, which is produced for a cell sample, measured. Because of this, and because of the earlier discussed limitations on first strand cDNA synthesis from cell sample RNAs, neither the absolute nor relative amounts of first strand cDNA can be known for each compared cell sample. In addition, the cell sample first strand cDNAs may differ significantly in nucleotide length. Similarly, for this process only rarely is the amount of double strand cDNA produced from the first strand cDNA, measured for each compared cell sample. However, the amount of cRNA produced in the final amplification step is very often measured for each compared cell sample cRNA. In addition, the average nucleotide length and nucleotide length profile for each compared cell sample's cRNA prep is often determined. Equal amounts of each compared cell sample cRNA prep are generally incorporated directly into the microarray assay hybridization solution. In addition, it is not unusual for the compared cell sample cRNA preps to differ significantly in average nucleotide length.

It is known that the cRNA synthesis efficiency from the double strand cDNA, and the composition or purity of the resulting cRNA prep, can be significantly affected by a variety of assay factors. Such variations in composition or purity can result in the comparison of cell sample cRNA preps, which contain quite different masses of hybridizable cRNA, even though equal masses of each cell sample cRNA prep are compared in the assay. In addition, different compared cRNA preps can have different average nucleotide lengths. It is known that for the overall process of producing cRNA from mRNA, the cRNA yield from a given amount of starting cell RNA can vary by threefold or more for different sources of cell RNA, and that the resulting cRNA nucleotide lengths are shorter than those of the cell mRNA templates.

For this process of producing compared cell sample cRNA preps, the EA Rule is practiced twice. Initially equal amounts of each cell sample RNA prep are utilized to start the process of producing the cRNA prep for each cell sample. Then at the end of the process, equal amounts of each cell sample cRNA prep are generally directly incorporated into the microarray assay hybridization solution. Here the cell sample mRNA prep is represented in the microarray assay by the mRNA equivalent cRNA prep. Prior art generally believes and practices that the mRNA equivalent cRNA prep faithfully represents the cell sample mRNA prep which was used to produce it, and that comparing equal amounts of two different cell sample cRNA preps is closely equivalent to comparing equal amounts of mRNA from the same two cell samples. Further, since the cRNA is produced from double strand cDNA, which is produced from single strand cDNA made from the cell sample mRNA prep, prior art generally believes and practices that the mRNA equivalent cDNA prep faithfully represents the single strand cDNA mRNA equivalent, and the double strand cDNA mRNA equivalent. However, there are indications that the cell sample cRNA preps do not always have the same representation and frequency as the cell sample RNA preps they are produced from (102, 132).

Prior art utilizes northern blot, dot blot, nuclease protection, and RT-PCR related methods in order to validate or corroborate microarray or RT-PCR related gene expression comparison results (133). These methods virtually always practice one or another version of the EA Rule. For these methods, the northern blot, dot blot, and nuclease protection assay results are derived from the direct comparison of cell T-RNA or mRNA in the assay. In contrast, as discussed the microarray assay results are obtained by the direct comparison of the mRNA equivalent, cDNA or cRNA, in the assay, while the RT-PCR related assay results are obtained by the direct comparison of the mRNA equivalent cDNA. This corroboration approach can be valid if the cell mRNA equivalents compared are representative of their respective cell mRNAs, and if the relative amounts of mRNA equivalents compared accurately reflects the relative amounts of the respective cell RNAs utilized to produce the mRNA equivalents.

Current Method for Determining the Absolute Amount of a Sample RNA or Equivalents Added to the Assay.

There is no general rule for determining the actual amount of a sample total RNA, mRNA, or equivalents, to compare in a gene activity assay. Ideally, enough sample RNA or equivalents should be added to ensure the detection of the least frequent mRNA type present in the sample total RNA, total mRNA, or equivalents. This would ensure the detection of the least active gene in the sample. Ideally then, the minimum amount of sample RNA which should be added to the gene activity assay, is that amount of total RNA, or total mRNA, or equivalents, which contains a just detectable amount of mRNA transcripts from the least active gene in the sample. In reality, it is often difficult, if not impossible, to conduct gene activity measurements under ideal conditions. This is especially true for mammalian gene activity comparisons. Because of the small genetic complexity and ready availability of adequate quantities of sample RNA, the ideal situation is often met or approximated in prokaryote and simple eukaryote gene activity comparisons. Unfortunately, this is not true for mammalian cell gene activity comparisons, where a very much larger genetic complexity, and a scarcity of many mammalian cell samples which greatly limits the amount of RNA available, combine to ensure that it is only rarely possible to meet the ideal requirement for addition of sample RNA to the assay (5). The result of this is that in mammalian cell gene activity comparisons, the amount of sample RNA available to add to the assay is very often not enough to ensure that the majority of the low abundance mRNAs are detectable, and often the low abundance mRNAs are not detectable at all. The mammalian low abundance mRNA represents the activity of ten thousand or so genes. From this, it follows that in many mammalian gene activity comparisons, a large number of actually active genes give a negative result in the assay. These negative results are then false negatives. These false negatives can be converted to true positives by adding a greater amount of sample RNA to the assay.

Independent Validation and Corroboration of Microarray Gene Expression Comparison Results.

Prior art believes and practices that once statistically significant microarray gene expression activities and ratios are established, it is important to validate the results using an alternate method of gene expression (22, 133-135). Currently such alternate methods include northern blotting, dot blot, ribonuclease protection assay, in situ hybridization, the various forms of reverse transcriptase polymerase chain reaction method (RT-PCR) method, and the differential display methods, and on occasion the Serial Analysis of Gene Expression (SAGE) method or the Massively Parallel Signature Sequencing (MPSS) method. Other gene expression analysis methods such as ELISA, hydroxyapatite, and other affinity column based methods are rarely used for this purpose. Any of these methods can be used to corroborate the existence of a microarray determined positive or negative gene activity. To corroborate a prior art microarray determined quantitative gene expression ratio for a gene, the northern blot, RT-PCR, or RNAase protection methods, are generally used.

Prior art non-microarray corroborative methods virtually always practice the earlier discussed EA Rule, which specifies that equal amounts of cell sample RNA, or equivalents, be compared in the assay. Prior art considers it important to compare equal amounts cell sample RNA and often incorporates added loading control polynucleotide molecules into the non-microarray corroborative assay in order to normalize the assay results for pertinent assay associated variables, including differences in the amounts of compared cell sample RNA, or equivalents. Prior art believes that non-microarray or corroborative assay results must be normalized in order to be biologically correct (31, 96). Prior art normalization of such non-microarray or corroborative assay results rely heavily on the use of putative housekeeping genes as internal controls for normalization (75, 109, 134). Prior art believes and practices that prior art normalized non-microarray results are biologically correct, and that it is valid to intercompare such normalized results to those obtained by other non-microarray or microarray methods. Often such comparisons of non-microarray and microarray results, and non-microarray of one type and non-microarray of another type results agree, and often the compared results do not agree. As an example, one study reported that for 17 different particular gene comparisons which were microarray measured to be significantly differentially expressed, only 8 were measured as being significantly expressed by they non-microarray corroborative method (64).

Prior Art Considered Assay Variables Associated with the Normalization of Prior Art Non-Microarray Gene Expression Analysis Results.

Some of the same prior art known assay variables are considered by the prior art for the normalization of prior art non-microarray gene expression analysis results. In addition, different non-microarray methods can be associated with different prior art known and considered assay variables. The prior art known assay variables which are considered by the prior art for the normalization of prior art SGDS mRNA transcript comparison assay results generated by each different non-microarray gene expression analysis methods, are discussed below.

Prior art dot blot, northern blot, and ribonuclease protection methods at times normalize for the assay variables associated with the amount of total RNA or mRNA compared, and the efficiency of hybridization of the LPN with the immobilized RNA. Prior art has used housekeeping genes for normalization of prior art dot blot, northern blot, and ribonuclease protection results.

Prior art RT-PCR and QRT-PCR methods at times normalize for assay variables associated with the amount of total RNA or mRNA compared, the amount of mRNA cDNA compared, the relative efficiency of the reverse transcriptase copying of the compared RNAs, and the relative efficiency of amplification of the cDNA by the DNA polymerase. RT-PCR and QRT-PCR has used both housekeeping genes and added exogenous internal standard molecules for normalization. Added exogenous standards often cannot control for the amount of RNA or cDNA compared, or the efficiency of reverse transcriptase copying of the input RNA'S, but prior art RT-PCR practice often believes that housekeeping gene mRNA's can control for these factors. Prior art has utilized both housekeeping gene mRNAs and exogenously added standard mRNAs in an effort to control for the efficiency of reverse transcriptase synthesis and the PCR amplification of the cDNA by the DNA polymerase.

Key Prior Art Beliefs and Practices for Microarray and Non-Microarray Gene Expression Analysis. The Representation and Frequency of RNA Transcripts and RNA Transcript Equivalents.

It will first be useful to discuss the representation and frequency of occurrence of each particular gene mRNA transcript type, which is present in a cell sample. This will be done in terms of the mRNA of a typical mammalian cell, but the discussion and definitions apply directly to cells and cell samples of all kinds, and to different types of rRNAs, tRNAs, miRNAs, siRNAs, snoRNAs, and any other known or unknown RNA which is present in a cell. A particular gene mRNA is represented in the total mRNA population of a cell or cell sample when at least one molecule of the particular gene mRNA is present in the cell or cell sample. For a typical mammalian cell, it has been reported that about 15,000 different particular gene mRNA types are present in the cell. The frequency of occurrence of a particular gene mRNA transcript in a cell or cell sample can vary greatly, depending on the gene. One particular gene mRNA can be represented by thousands of mRNA transcript copies per cell, while a different gene mRNA transcript may be present only once per cell. The frequency of occurrence of a particular gene mRNA transcript in a cell or cell sample, is here defined in terms of the ratio of (the number of the particular gene mRNA molecules per cell)÷(the number of mRNA molecules of all kinds in the cell). Alternatively, said frequency is equal to the ratio of (the number of the particular gene mRNA molecules per cell sample)÷(the total number of mRNA molecules of all kinds in the cell sample). These ratios are equivalent to the ratio of (the moles of a particular gene mRNA per cell or cell sample)÷(the moles of mRNA molecules of all kinds in a cell or cell sample). The frequency of occurrence of a particular gene mRNA in a cell or cell sample, is herein termed the particular gene mRNA mole frequency, or mRNA Fmole. For a single cell in a cell sample, the cell mRNA Fmole for a particular gene mRNA, does not necessarily equal the cell sample mRNA Fmole for the same gene's mRNA.

The frequency of occurrence of a particular gene mRNA transcript in a cell or cell sample can also be defined in terms of the ratio of (the mass of all of a particular gene's mRNA molecules which are present in a cell or cell sample)÷(the mass of all mRNA molecules of all kinds which are present in a cell or cell sample). Herein this is termed the cell mRNA mass frequency or mRNA Fmass, or the cell sample mRNA Fmass. For a particular gene mRNA in a cell or cell sample, the Fmole does not necessarily equal the Fmass.

Virtually all prior art microarray and non-microarray gene expression analyzes routinely practice and believe the validity of the following assumptions. The representation and frequency of occurrence of each particular gene mRNA present in the intact cell or cell sample, is essentially identical to the representation and frequency of occurrence of each particular gene mRNA present in the total RNA (T-RNA) prep isolated from the cell or cell sample, and is also essentially identical to the representation and frequency of each particular gene mRNA present in the mRNA prep isolated from the cell or cell sample T-RNA prep. In other words, it is assumed that isolation of the cell or cell sample total RNA and mRNA does not result in a significant change in the representation or frequency of occurrence of particular gene mRNAs, relative to the intact cell or cell sample. Further, it is assumed that the process of producing cell or cell sample mRNA LPN preparations from cell or cell sample total RNA or total mRNA does not result in a significant change in the representation or frequency of occurrence of particular gene mRNAs, relative to the intact cell or cell sample. For a particular gene mRNA which is present in isolated T-RNA or mRNA prep: the Fmole of the mRNA in the T-RNA prep is equal to the ratio of (the moles of particular gene mRNA present in the T-RNA)÷(the moles of mRNAs of all kinds which are present in the T-RNA prep); the Fmole of the mRNA in the isolated mRNA prep is equal to the ratio of (the moles of particular gene mRNA present in the isolated mRNA prep)÷(the moles of mRNA of all kinds which are present in the isolated mRNA prep); the Fmass of the mRNA in the isolated T-RNA prep is equal to the ratio of (the mass of all of the particular gene mRNA present in the T-RNA prep)÷(the mass of all mRNA molecules of all kinds which are present in the T-RNA prep); the Fmass of the mRNA in the isolated mRNA prep is equal to (the mass of all of the particular gene mRNA molecules which are present in the isolated mRNA prep)÷(the mass of all mRNA molecules of all kinds which are present in the isolated mRNA prep). The basic F assumptions then, specify that for a particular gene mRNA which is present in a cell or cell sample, (the Fmole and Fmass for the mRNA in the cell or cell sample)=(the Fmole and Fmass for the mRNA in the isolated cell or cell sample T-RNA prep)=(the Fmole and Fmass for the mRNA in the mRNA isolated from the cell or cell sample T-RNA).

Only rarely is cell sample T-RNA or isolated mRNA directly compared in microarray and RT-PCR related gene expression comparison assays. Instead, the mRNA equivalents cDNA or cRNA are directly compared. Such cDNA or cRNA mRNA equivalents are produced from the compared cell sample's T-RNA or isolated mRNA preps. Prior art generally assumes that the process of producing the cDNA or cRNA prep does not result in a significant change in the representation and frequency of a particular gene mRNA in the cDNA or cRNA prep, relative to the representation and frequency of the particular gene mRNA in the cell sample T-RNA or isolated mRNA prep which was used to produce the cDNA or cRNA prep.

Prior art believes and practices that these R and F assumptions must be valid for at least a portion of each particular gene mRNA, in order to obtain microarray and non-microarray gene comparison assay results which are biologically accurate and interpretable. The validity of each of these key beliefs is discussed later. Note that the above discussion is directly applicable to all different types of RNA which are present in a cell sample, and to SGDS, DGDS, and DGSS RNA transcript comparisons of all kinds.

Key Prior Art Beliefs and Practices for Microarray and Non-Microarray Gene Expression Analysis. Three Tacit Assumptions.

The above-discussed R and F requirements are necessary for all prior art microarray and non-microarray gene expression analyzes and gene comparison analyzes. Prior art believes and practices that prior art produced particular gene mRNA transcript expression analysis assay abundance results and particular gene mRNA transcript comparison analysis N-DGER results, are biologically accurate within the accuracy of the assay, and do not need further normalization. Many prior art microarray and non-microarray assays claim a measurement accuracy of ±1.2-2 fold for the assay result. In order for this prior art belief and practice to be valid, unknown to the prior art, each of the three tacit assumptions which is pertinent for the prior art assay, must be valid. Alternatively, and also unknown to the prior art, biologically accurate prior art particular gene assay results may occur when one or more of these tacit assumptions is invalid, if the effect of the invalidity of one or more tacit assumptions on the biological accuracy of the assay results, is cancelled by the effect of the invalidity of one or more different tacit assumptions or other assay factors, on the biological accuracy of the assay results. This is an unlikely event and it is assumed that such events occur only rarely and can be ignored for this discussion. The discussion for each separate tacit assumption will assume that the other two tacit assumptions are valid. The phrase, unknown to the prior art, is used because prior art does not determine or take into consideration during the normalization process, the validity of these tacit assumptions for an assay. In the above context, each tacit assumption is described in terms of what the prior art must assume about the tacit assumption, in order to obtain biologically accurate particular gene mRNA transcript number or abundance values, and SGDS particular gene mRNA transcript comparison N-DGER values.

Tacit assumption one has more than one form. For an EA rule associated prior art microarray or non-microarray assay which compares cell sample RNA directly, a prior art measured particular gene comparison N-DGER value can be biologically correct only when the amount of T-RNA or mRNA per cell is the same for the compared cell samples. For an EA rule associated prior art microarray assay which compares cell sample cDNA or cRNA preps, a prior art measured particular gene comparison N-DGER value can be biologically correct only when the amount of cDNA or cRNA which represents a cell sample (i.e., a cRNA cell equivalent or CE), is the same for each compared cell sample cDNA or cRNA prep. These tacit assumptions are pertinent for all prior art microarray and non-microarray SGDS mRNA transcript comparison assays. In addition, such assumptions can be pertinent for SGDS and DGDS RNA transcript comparisons for RNAs of any type. However, the assumption is not pertinent for microarray DGSS RNA transcript comparison assays, or DGSS RNA transcript equivalent comparison assays.

Tacit assumption two specifies that for prior art microarray and non-microarray mRNA transcript expression analysis assays, a prior art measured particular gene mRNA abundance value can be biologically correct only when the cell sample RNA isolation efficiency is equal to one. This aspect of assumption two is also pertinent for particular gene RNA transcript expression analysis assays for any RNA type. Tacit assumption two also specifies that for those prior art SGDS mRNA transcript comparison assays which derive particular gene comparison DGER values from assay measured particular gene mRNA abundance values, a prior art measured SGDS particular gene mRNA transcript comparison assay N-DGER value can be biologically correct only when the cell sample RNA isolation efficiencies are the same for the compared cell sample RNA preparations. This tacit assumption is also pertinent for SGDS and DGDS RNA transcript gene expression comparison assays for RNAs of any type, which determine abundance measurement derived N-DGER values. However, the assumption is not pertinent for microarray and certain non-microarray DGSS particular gene RNA transcript expression comparison assays for any RNA type. Herein a cell sample RNA isolation efficiency is termed the RIE, and the ratio of compared cell sample RNA preparation RIE values, is termed the RIE ratio or RIER.

Tacit assumption three concerns the efficiency of cDNA or cRNA synthesis for prior art microarray assays, and the efficiency of cDNA synthesis and the efficiency of cDNA amplicon amplification for prior art RT-PCR assays. Since virtually all prior art microarray and RT-PCR gene RNA transcript expression analysis assays involve the SGDS comparison of mRNA transcripts, prior art tacit assumption three will be discussed in terms of SGDS comparisons of cell sample mRNA transcripts, unless otherwise noted. Herein, a cell sample cDNA or cRNA prep synthesis efficiency is termed a cDNA SE or cRNA SE. A cell sample cDNA SE value is equal to, (the number of cell sample cDNA cell equivalents produced in the RT synthesis step)÷(the number of cell sample T-RNA or mRNA cell equivalents present in the RT synthesis step). A cell sample cRNA SE value is equal to, (the number of cell sample cRNA cell equivalents produced in the cRNA synthesis step)÷(the number of cell sample cDNA template cell equivalents present in the cRNA synthesis step). The SE ratio for a cell sample cDNA or cRNA prep comparison, is termed the cDNA SER or cRNA SER. Note that for a particular gene mRNA transcript which is represented in the cell sample cDNA prep, the overall cell sample cDNA prep SE value is equal to, (the number of a particular gene mRNA transcript cDNA equivalent molecules produced in the synthesis step)÷(the number of particular gene mRNA transcript molecules present in the synthesis step), when the R and Fmole assumptions are valid. Similarly, the cell sample cRNA prep SE value is equal to, (the number of particular gene mRNA transcript cRNA equivalent molecules produced in the cRNA synthesis step)÷(the number of particular gene mRNA transcript DS cDNA equivalent molecules present in the cRNA synthesis step), when the R and Fmole assumptions are valid. Therefore, for any cDNA or cRNA molecule prep produced from a known number of exogenous standard nucleic acid molecules, the standard cDNA prep SE value is equal to, (the number of standard mRNA transcript cDNA equivalent molecules produced in the cDNA synthesis step)÷(the number of standard mRNA transcript molecules present in the cDNA synthesis step), and the standard cRNA prep SE value is equal to, (the number of standard mRNA transcript cRNA equivalent molecules produced in the cRNA synthesis step)÷(the number of standard mRNA transcript cDNA equivalent molecules present in the cRNA synthesis step), when the R and Fmole assumptions are valid. Because of this, cell sample cDNA and cRNA SE values can be directly compared to particular gene mRNA transcript or standard mRNA transcript cDNA and cRNA SE values. These relationships are pertinent for both microarray and non-microarray RT-PCR related prior art and other assays. Note that a cell sample cDNA prep SE assay value is almost always significantly less than one, and the cell sample cRNA prep SE assay value is almost always equal to much greater than one. Typically, the cRNA SE equals 10 to thousands, while the cDNA SE equals from 0.1 to 0.5.

A cell sample particular gene cDNA molecule or a standard cDNA molecule, which can be detected by PCR amplification, is termed a particular gene or standard cDNA amplicon equivalent molecule, or a particular gene cDNA or standard cDNA AE molecule. A cell sample particular gene mRNA transcript molecule or a standard mRNA transcript molecule, which can produce a cDNA AE molecule, is termed an RNA or mRNA AE molecule. For RT-PCR assays, it is useful to define the cDNA synthesis efficiency in terms of the efficiency of synthesis of particular gene and standard cDNA AE molecules from cell sample particular gene mRNA transcript or standard mRNA transcript AE molecules. Here, the particular gene or standard cDNA AE synthesis efficiency is termed the particular gene or standard AE•SE. The AE•SE value for a cell sample particular gene mRNA transcript cDNA AE is equal to the cell sample SE value or, (the number of the particular gene mRNA transcript cDNA AE molecules produced in the assay RT step)÷(the number of particular gene mRNA AE transcript molecules present in the amount of cell sample RNA which is present in the RT step). The number of particular gene RNA transcript molecules which is present in a given amount of cell sample RNA, is herein termed the cell sample RNA transcript number or RNA AE transcript number, or more simply the particular gene RN or AE•RN. The AE•SE value for a standard RNA transcript cDNA AE is equal to the standard SE value or, (the number of standard RNA transcript AE cDNA molecules produced in the assay RT step)÷(the number of standard RNA transcript AE molecules which is present in the assay RT step). The number of standard RNA AE transcript molecules present in the assay RT step is termed the standard RNA AE transcript number, or standard AE•RN. For the particular gene and standard, the number of RNA transcript cDNA AE molecules produced in the assay RT step is herein termed either the particular gene cDNA or the standard AE cDNA transcript number, or AE•CN. Note that for a microarray assay the particular gene or standard AE•RN and AE•CN parameters are designated the particular gene and standard RN and CN.

The AE•SE value for a particular gene mRNA transcript cDNA prep or a standard mRNA transcript cDNA prep is then equal to, (the particular gene or standard AE•CN value)÷(the particular gene or standard AE•RN value), or (AE•CN)÷(AE•RN). For a cell sample particular gene comparison the AE•SE ratio is then equal to, (one cell samples particular gene AE•SE value)÷(the other cell samples particular gene AE•SE value), and is termed the AE•SER.

For prior art RT-PCR assays, the third tacit assumption also involves the efficiency of AE cDNA amplification in the assay PCR amplification step. For particular gene and standard AE cDNAs the AE amplification efficiency is termed the AE•AE, and the ratio of compared particular gene or standard AE•AE values is termed the AE•AER. For a particular gene or standard AE cDNA amplification step, the AE•AE value is equal to, (the number of particular gene or standard amplicon molecules produced in the PCR amplification step during a known number of amplification cycles)÷(the number of particular gene or standard amplicon molecules which would be produced during the same known number of amplification cycles when the PCR E value equals one). The PCR E value is the classic amplification efficiency parameter (117). For an E value of one, each amplicon molecule will produce two amplicon molecules in one PCR cycle, and for an E value of 0.7, each amplicon molecule will produce 1.7 amplicon molecules per PCR cycle. Here, when the E value equals one, the AE•AE value will equal one. In this context, (the particular gene or standard AE•AE value)=(1+the particular gene or standard assay value for E)^{N}÷(2)^{N}, where N equals the number of assay amplification cycles.

It is known that the cDNA SE values and cDNA AE•SE values for prior art microarray and RT-PCR assay cell sample, standard, and particular gene cDNA preps and AE cDNA preps are almost always equal to significantly less than one (103, 106). These cDNA SE values are generally in the range of 0.1 to 0.5, and are only rarely determined by prior art microarray or RT-PCR practice. Further, it is known that the SE values and AE•SE values for different compared cell samples of the same and different types can vary significantly, and SE or AE•SE differences of twofold or more would not be surprising for compared cell samples of the same type or different type. Prior art does not determine the cDNA SE, cRNA SE, or cDNA AE•SE values for a gene expression analysis or gene expression analysis comparison assay.

Prior art RT-PCR practice often assumes a value of one or nearly one for the particular gene and/or standard assay AE•AE values. Prior art reported PCR and RT-PCR particular gene and standard assay values for E generally vary from values of 0.7 to 0.9 (104, 106). This translates into assay AE•AE values, which vary from 0.008 to 0.21 for a 30 cycle PCR reaction. Note that at a particular E value the assay AE•AE value varies with N. A large number of assay factors is known to cause the assay AE•AE values to vary significantly. Prior art RT-PCR and PCR practice also often assumes that the assay ALGAE values for compared particular gene cDNA preps, compared standard cDNA preps, and compared particular gene and standard cDNA preps, are the same or nearly the same for an assay. Prior art only rarely determines the cDNA ALGAE assay values for RT-PCR assays. Prior art further believes and practices that because of the known variability which is associated with assay AE•AE values, it is necessary to utilize standards in the assay in order to obtain accurate gene expression results for single and compared cell sample analyzes.

The prior art third tacit assumption takes different, but related, forms for different prior art microarray and RT-PCR gene expression analysis and gene expression comparison analysis assays. These are discussed below. For simplification and clarity the discussion of each form of the third tacit assumption will assume that the first and second tacit assumptions are valid, and that the prior art produced gene expression analysis or comparison result is validly and correctly normalized for all assay pertinent assay variables except those associated with the third tacit assumption. In other words it is assumed that only the validity of the third tacit assumption can affect the biological accuracy of the prior art result. In addition, it is assumed that the standard prior art EA Rule practice is used for the assay to determine the amount of each compared cell sample total RNA or mRNA to use in the RT step of the assay.

For prior art microarray cell sample mRNA transcript comparison assays the third tacit assumption specifies the following. A prior art microarray particular gene mRNA transcript cDNA comparison assay measured N-DGER value can be biologically accurate only when the cDNA SEs of the compared cell sample cDNA preps are the same. This tacit assumption is also pertinent for all SGDS and DGDS microarray particular gene RNA transcript comparison assays for all RNA types. However, this third tacit assumption is not pertinent for DGSS microarray RNA transcript comparisons. Note that the third tacit assumption is not generally pertinent for prior art cell sample cRNA comparison assays where the cRNA SE values for each compared cell sample cRNA prep is significantly greater than one. Note further that for such cRNA prep comparisons the EA Rule is generally used to determine the relative amount of each cell sample cRNA to compare in the assay.

Because of the variability which is known to be associated with prior art PCR and RT-PCR cell sample and cell sample comparison gene expression analysis assay results, prior art believes and practices that the use of one or more assay mRNA and/or DNA standards is necessary in order to obtain accurate gene expression analysis results for the assays. The RT-PCR associated third tacit assumption is complicated by the use of standard mRNAs and/or standard DNAs in the assay. Each standard mRNA associated with a prior art RT-PCR assay is associated with a standard AE•SE value and a standard AE•AE value, while each standard DNA is associated with a standard DNA AE•AE value. When standards are used for the RT-PCR assay, each particular gene expression analysis or analysis comparison is associated with one or more mRNA and/or DNA standards, and the AE•SE and AE•AE values for both the particular gene and the standards can influence the biological accuracy of an RT-PCR measured particular gene AE•RN value (i.e. the assay measured AE-CN value) for the amount of cell sample RNA put into the assay RT step, and a particular gene N-DGER value for a cell sample comparison.

The RT-PCR related third tacit assumption is complex and varies for different prior art RT-PCR assay types. For a particular prior art RT-PCR assay type the third tacit assumption is defined in terms of, the assay associated particular gene AE•SE and AE•AE values and the interaction between these values, and the assay associated standard AE•SE and AE•AE values and the interaction between these values, and the interaction between the assay associated particular gene AE•SE and AE•AE values and standard AE•SE and AE•AE values, for the same RT-PCR assay. Note that for prior art RT-PCR assays, which include standards, the third tacit assumption definition includes the interactions between the particular gene and standard assay AE•SE and AE•AE values associated with the assay. The third tacit assumption associated with a particular prior art RT-PCR assay design, is then defined in terms of the assay associated particular gene and standard AE•SE and AE•AE values, and the interaction between these values which is required in order for the prior art RT-PCR particular gene expression analysis or particular gene expression comparison assay results to be biologically accurate as the prior art believes and practices, and not require normalization for the assay variables associated with the third tacit assumption. In this context, what the prior art must assume in order for the prior art RT-PCR assay measured particular gene AE•CN and N-DGER values to be biologically correct, is incorporated into the third tacit assumption. Various third tacit assumptions associated with the different prior art RT-PCR assay types are discussed below.

Prior art RT-PCR assay analyzes are designed to determine a quantitative measure of the AE•RN value for one or more particular gene mRNA transcripts which are present in a cell sample RNA prep. Prior art occasionally converts such particular gene AE•RN values to particular gene mRNA transcript abundance values for the cell sample. Prior art often compares particular gene AE•RN values from different cell samples in order to determine an SGDS particular gene comparison N-DGER value. Prior art occasionally compares particular gene mRNA transcript abundance values from different cell samples in order to determine an SGDS particular gene comparison N-DGER value. Here the discussion will focus on the prior art RT-PCR determination of, and biological accuracy of, particular gene AE•RN values, as well as on the prior art RT-PCR determination of and biological accuracy of SGDS particular gene comparison N-DGER values derived from prior art assay determined particular gene AE•RN values.

For prior art RT-PCR assays, which do not involve the use of a standard for the assay, the third tacit assumption specifies the following. A prior art measured particular gene AE•RN value can be biologically accurate only when the particular gene AE•SE and AE•AE assay values are both equal to one. In addition, a prior art measured particular gene comparison N-DGER value can be biologically correct only when the product of, (particular gene AE•SER value)×(particular gene AE•AER value), is equal to one.

For prior art RT-PCR assays which include a DNA standard for the PCR amplification step, but do not include an mRNA standard for the assay RT step, the third tacit assumption specifies the following. A prior art RT-PCR measured particular gene AE•RN value can be biologically accurate only when the product of, (the particular gene AE•SE assay value)×(PG/S AE•AER assay value) is equal to one. Here, the ratio of the particular gene (PG) and standard (S) AE•AE assay values is termed the PG/S AE•AER. In addition, a prior art RT-PCR assay measured particular gene comparison N-DGER value can be biologically correct only when the product of (the PG AE•SER)×(the PG AE•AER÷S AE•AER), is equal to one.

For prior art RT-PCR assays, which use an exogenous mRNA transcript standard for determining a quantitative measure for a particular gene AE•RN value in an assay, it will be useful to define the term PG/S AE•SER. The PG/S AE•SER for a cell sample RT-PCR analysis is equal to the ratio of, (the particular gene (PG) AE•SE assay value)÷(the standard AE•SE assay value). For such prior art RT-PCR assays the third tacit assumption specifies the following. A prior art RT-PCR measured particular gene AE•RN value for a cell sample can be biologically accurate only when the product of, (the assay PG/S AE•SER value)×(the assay PG/S AE•AER value), is equal to one. In addition, for prior art RT-PCR SGDS particular gene mRNA transcript comparison assays which use exogenous standard or endogenous true housekeeping gene standard mRNA transcripts, the assay measured particular gene comparison N-DGER value can be biologically accurate only when the ratio of, (the PG/S AE•SER value×the PG/S AE•AER value product for one cell sample)÷(The PG/S AE•SER value×PG/S AE•AER value product for the other compared cell sample) is equal to one.

Other forms of the third tacit assumption exist. The above described third tacit assumptions for microarray and RT-PCR assays are also pertinent for SGDS, DGDS, and DGSS particular gene RNA expression comparisons for all RNA types.

The validity of each of the three above described tacit assumptions for prior art microarray and non-microarray assays is discussed in later sections.

Other Key Assumptions and Prior Art Microarray and Non-Microarray Assay Beliefs and Practices.

In addition to the above discussed three tacit assumptions, other prior art beliefs and practices and assumptions which are essential for the prior art interpretation and analysis of prior art measured microarray and non-microarray gene expression analysis results include the following. (i) For a particular gene comparison assay, (the particular gene T-DGER) value)=(the particular gene ACR value), and (the particular gene assay measured NASR value)=(the particular gene ACR value). (ii) The earlier discussed key normalization assumptions. (iii) for a particular cell sample gene expression analysis or comparison, a microarray measured N-DGER value can be directly compared to a non-microarray measured result in order to corroborate the microarray result. (iv) During the first strand cDNA synthesis step, little or no second strand cDNA synthesis occurs. (v) The amount of cell sample T-RNA or mRNA or cDNA or cRNA present in the assay hybridization solution or PCR amplification solution is accurately quantitated. (vi) For an assay the measured assay signal is directly proportional to the amount of input T-RNA or mRNA or cDNA or cRNA for the assay. The validity of these prior art practices and assumptions will be discussed in later sections.

The SAGE and Other Clone Counting Methods of Gene Expression Analysis and Comparison.

The various forms of the SAGE and other clone counting methods including the MPSS method, are well described in the literature. A clone counting method analysis of a cell sample involves the following. (i) Isolation of cell sample T-RNA or mRNA. (ii) Using oligo dT priming to produce a cell sample cDNA prep. (iii) Cloning the entire cell sample cDNA prep to create a cloned cell sample cDNA prep library. (iv) Sampling the library clones in a statistically significant manner in order to determine the presence of particular gene mRNA tags and a measure of the total number of particular gene mRNA tags of all kinds which are present in the library, and their identity. The total number of mRNA tags of all kinds detected in a clone library is believed to represent the number of total mRNA molecules of all kinds, which were present in the cell sample RNA. (v) The frequency of occurrence of each different particular gene clone tag in the library is measured in terms of, (the number of identified cloned tags for a particular gene mRNA)÷(the total number of identified particular gene mRNA tags of all kinds). Here this is termed the particular gene mRNA tag frequency, or the particular gene mF for the cell sample of interest. Prior art typically adjusts the measured mF values assay variables. These include, but are not limited to, sequencing error and sampling statistics considerations. Prior art believes and practices that such a particular gene mF value represents the ratio of (the number of particular gene mRNA molecules)÷(the total number of particular gene mRNA molecules of all kinds), which is present in the intact sample cells and the isolated cell sample T-RNA or mRNA preps. (vi) For a clone counting method cell sample comparison assay, the ratio of (the particular gene mF value for one cell sample)÷(the mF value for the same particular gene for the other compared cell sample), is termed the particular gene mF ratio or mFR, for the cell sample comparison. Prior art believes and practices that such a measured particular gene mFR value is equal to the particular gene T-DGER value for the cell sample comparison. Prior art further believes and practices that such a measured particular gene comparison mFR value, can validly be used to corroborate an N-DGER value for the same particular gene comparison obtained using a microarray or non-microarray method.

The above described prior art beliefs and practices concerning clone counting measured particular gene mF and particular gene comparison mFR values, are valid only if certain prior art assumptions concerning the clone counting method process are valid. These are described below.

The following assumptions must be valid in order for the above described prior art clone counting method practice and belief to be valid. (i) For a produced cell sample mRNA clone tag library, the earlier discussed R and Fmole assumptions must be valid for at least the clone counting method pertinent portion of each mRNA molecule of any kind which is present in the intact cells of the analyzed cell sample. Such a pertinent portion of an mRNA molecule is the 3′ end portion adjacent to the poly A tract. (ii) For a produced cell sample clone tag library, the earlier discussed first tacit assumption must be valid for the compared cell sample mRNA populations. (iii) For a clone counting method measured particular gene mRNA abundance value, or particular gene comparison DGER value determined from compared particular gene mRNA abundance values, the earlier discussed second tacit assumption must be valid for the compared cell samples. These assumptions are also pertinent for particular gene expression SGDS, DGDS, and DGSS comparisons.

For a prior art cell sample cloned tag library comparison, the absolute total number of individual particular gene tags of all kinds sampled for each cell sample is determined by clone sample statistics. Such sampling statistics also contribute to the assay error associated with each SAGE or other clone counting method measured particular gene mF and mFR values. Since prior art believes and practices that the mRNA content per cell is the same for the compared cell samples, generally approximately equal numbers of library tags are compared.

Note that rRNA, tRNA, miRNA, siRNA, and snoRNA which is present in a cell is not polyadenylated and therefore cannot be analyzed by standard SAGE practice unless an efficient method of polyadenylating these RNAs is available. Absent this, these RNAs can be analyzed by other methods.

Note further that the MPSS clone counting method involves the PCR amplification of all of the particular gene mRNA double strand cDNA equivalent molecules present in a cell sample mRNA transcript cDNA prep. As a result, the MPSS based assay has associated with it the assay variables associated with PCR amplification.

### SUMMARY OF THE INVENTION

The present invention is based on the discovery that nearly all nucleic acid-based assays currently used include significant assay factors which are not normalized, and which can dramatically affect the results and interpretation of the assays. As a result, an aspect of this invention involves identifying and normalizing such additional assay factors and/or correctly normalizing for recognized assay factors. An important result of improving on current assay practices in this manner is improvement in the accuracy and/or interpretability of assay results, among others. In particular, this invention provides dramatic improvements in the performance and reliability of gene expression assays, profiling, gene expression profile comparisons, and other such assays and applications.

Thus, in a first aspect, the invention provides a method for producing improved particular gene (PG) RNA transcript expression analysis assay results for a PG RNA transcript expression analysis assay for a cell sample RNA transcript preparation or equivalent nucleic acids derived therefrom, and/or a PG RNA transcript expression comparison analysis assay for compared cell sample RNA preparations or equivalent nucleic acids derived therefrom. The method involves normalizing the assay measured PG RNA transcript expression results for an analyzed cell sample and/or the assay measured PG RNA transcript expression comparison results for the compared cell samples, for one or both of (a) one or more pertinent assay variable-associated unconsidered normalization factors (UNFs) using pertinent assay values for individual UNFs or UNF combinations or both, and (b) one or more pertinent improved (e.g., validly determined) considered normalization factor (CNF) assay values whose values are known to be improved (e.g., validly determined), using pertinent assay values for individual CNFs or CNF combinations or both, such that the normalizing produces assay results which are known to be improved in normalization and/or in interpretability relative to such RNA transcript expression assay results and PG RNA transcript expression comparison assay results obtained by prior assay and normalization practices.

In particular embodiments, at least 1, 2, 3, 4, 5, 6, 7, 8, 9, 10, or more such UNFs are utilized, and/or at least 1, 2, 3, 4, 5, or more improved (e.g., validly determined) CNFs are utilized.

In particular embodiments in which UNFs and/or CNFs are utilized, the utilized UNF(s) and/or improved CNFs are each different from a sample cell number (SC) or sample cell number ratio (SCR); a PAF or PAFR; a MLD or MLDR; a PL-HKR; PS-HKR; a PSA or PSAR; a PSS or PSSR; a SBN or SBNR; a SSA or SSAR; a LLS or LLSR; C-HKR; a STM or STMR; a spatial CNF; a print tip CNF; a print plate CNF; an intensity CNF; a scale CNF; no CNFs are used.

In particular embodiments, the method also includes identifying one or more UNFs and/or CNFs which are pertinent for the assay and/or obtaining an assay value for 1, 2, 3, 4, 5, or more CNFs and/or UNFs, or for a combination of two or more identified pertinent CNFs and/or UNFs. In some embodiments, the method includes determining that values for one or more particular CNFs can be improved (e.g., validly determined) and/or determining that a particular CNF is a improved CNF, an invalid CNF, or an uncertain validity CNF and/or validly determining an assay value (e.g., an improved assay value) for one or more, or for a combination of two or more, such CNFs. Combinations of CNFs and/or UNFs can include, among others, each combination of UNFs, CNFS, or UNFs and CNFs together from the UNFs and CNFs described herein taken 2, 3, 4, 5, 6, 7, or more at a time.

In some embodiments in connection with a CNF, the method includes determining that the compared cell sample measured total mRNA content per cell or the total number of mRNA molecules per cell (STM) values differ significantly (e.g., at least 1.5, 1.6, 1.7, 1.8, 1.9, or 2.0 fold, or more), determining that the measured difference is not primarily due to a greater number of mRNA molecules from genes which are expressed only in the compared sample which is associated with the larger measured value, and determining that the difference in compared measured values is not primarily due to an increase in mRNA copies per cell in only one of the compared samples for one or more genes which are expressed in both compared samples. If each of those conditions are true, then the CNF is an invalid CNF.

Likewise in some embodiments in connection with a CNF, the method also includes identifying one or more CNFs which are pertinent for said assay and which are of uncertain validity, e.g., by determining for each compared cell sample the total mRNA content per cell or the total number of mRNA molecules of all kinds per cell, and comparing the determined values, where if the compared determined values are significantly different then the CNF is a CNF of uncertain validity.

In particular embodiments, e.g., where a CNF has been determined to be of uncertain validity, the method includes validly determined CNF values are obtained by utilizing a valid normalization process.

Also in particular embodiments, the method also includes incorporating multiple different replicated individual RNA or DNA standards or both into the assay, performing the assay and determining the assay results, and utilizing the assay results from the RNA or DNA standards or both to determine (e.g., validly determine) one or more CNF values for the assay without reliance on prior usual normalization assumptions.

Likewise, in particular embodiments the method includes validating a prior art normalization process for the assay, and utilizing the validated prior art normalization process to determine one or more pertinent improved (e.g., valid) CNF values for the assay. For example, the validating can concern a prior art normalization method for an assay which relies on the usual prior art normalization assumptions to determine whether the method can be utilized for said assay to produce improved (e.g., validly determined) CNF values, e.g., by determining that the STM value for each cell sample is approximately the same (e.g., less than 1.5, 1.4, 1.3, 1.2, or 1.1 fold difference, that is (value 1)/value 2) is less than 1.5 or other specified value) for an assay which compares cell sample mRNA, determining for the assay that the total number of the different particular mRNA genes which are expressed in both compared cell samples is approximately the same, where if the specified conditions are met, then for that assay one or more of the necessary usual prior art normalization assumptions are valid, and one or more prior art normalization methods which rely on those necessary normalization assumptions can be used to determine improved (e.g., valid) CNF values for the assay. In view of the fact that for many assay methods the normalization methods used are not, or cannot, be known to be valid, in some embodiments the method includes determining that a prior art normalization method is valid.

The assay can be of any of a number of different types. Thus, in certain embodiments, the assay is or includes a microarray assay (usually an oligonucleotide microarray, such as a cDNA microarray), or a lower density array assay; a RT-PCR assay (or other PCR-based assay); a nuclease protection assay; a clone counting or SAGE assay; an ELISA assay; an affinity medium separation assay, such as an assay using hydoxyapatite as a separation medium (e.g., in column format).

The assay (e.g., gene expression analysis assays) may be configured for various scales. Thus, in particular embodiments, the assay is a high throughput assay (e.g., suitable for performing at least 10000, 20000, 30000, 50000, or more assay determinations in a single assay run (e.g., using a high density microarray which typically requires about 24 hours of assay operation), a medium throughput assay (e.g., suitable for performing at least 500, 1000, 2000, 3000, 5000, or up to 9999 assay determinations in a single assay run (e.g., using a medium density microarray which typically requires about 24 hours of assay operation), or a low throughput assay (e.g., suitable for performing 1-499 assay determinations in a single assay run (e.g., using a low density microarray or RT-PCR or nuclease protection or other method, which typically require about 2-24 hours of assay operation depending on the assay throughput, type, and specific configuration).

Different levels of normalization improvement may be useful Thus, in certain embodiments, the improved assay result is validly and completely normalized for all assay pertinent UNFs and/or assay pertinent CNFs; the improved assay result is validly and completely normalized for all recognized assay pertinent UNFs and/or assay pertinent CNFs; the improved assay result is validly normalized for all assay pertinent UNFs and/or assay pertinent CNFs which have significant effect; the improved assay result is validly normalized for at least one, but less than all, assay pertinent UNFs and/or assay pertinent CNFs, thereby producing an improved PG assay result which is incompletely normalized for all assay pertinent UNFs and CNFs.

In particular embodiments, the unconsidered assay variable associated UNFs include one or more of the UNFs A•SC, A•SCR, R•SC, R•SCR, PAF, PAFR, MLD, MLDR, PL-HKR, PS-HKR, PSA, PSAR, PSS, PSSR, LLS, LLSR, SBN, SBNR, SSA, SSAR, STM, and STMR. Such combinations include each combination of the listed UNFs taken 2, 3, 4, 5, 6, 7, 8, 9, 10, . . . 22 at a time. Likewise, in particular embodiments, the prior art known and considered assay variable associated CNFs include one or more of the CNFs sampling statistics, sequencing error, C-HKR, spatial, print tip, print plate, intensity, scale, AE•SE, AE•SER, AE•AE, AE•AER. Such combinations include each combination of the listed CNFs taken 2, 3, 4, 5, 6, 7, 8, 9, 10, 11, or 12 at a time. The UNFs and CNFs may also be combined, e.g., one or more UNFs and one or more CNFs, which includes, or example, all combinations of indicated combinations of UNFs with indicated combinations of CNFs.

In certain embodiments is a SGDS assay; a DGDS assay; a DGSS assay; a type 1 assay; a type 2 assay; the assay involves use of a directly labeled polynucleotide (LPN, e.g., RNA, DNA, cDNA, cRNA); the assay involves use of an indirectly labeled polynucleotide.

In further cases the assay is a microarray assay which which analyzes cell sample RNA transcripts or their equivalent cDNA or cRNA nucleic acids, and in particular embodiments is a SGDS or DGDS type 1 or type 2 direct or indirect label LPN assay, and the CNFs include one or more or all of C-HKR, spatial, print tip, print plate, intensity, scale, or the UNFs include one or more or all of A•SC, A•SCR, R•SC, R•SCR, PAF, PAFR, MLD, MLDR, PL-HKR, PS-HKR, PSA, PSAR, PSS, PSSR, or both the CNF and UNF as specified are utilized; a SGDS or DGDS type 1 direct label LPN assay, and the CNFs include one or more or all of C-HKR, spatial, print tip, print plate, intensity, scale, or the UNFs include one or more or all of A•SC, A•SCR, R•SC, R•SCR, PAF, PAFR, MLD, MLDR, PL-HKR, PS-HKR, PSA, PSAR, PSS, PSSR, or both the CNF and UNF as specified are utilized; a DGSS type 1 direct label LPN assay which analyzes cell sample RNA transcripts or their equivalent cDNA or cRNA nucleic acids, and the CNFs include one or more of C-HKR, spatial, print tip, print plate, intensity, scale, or the UNFs include one or more of A•SC, R•SC, PAF, PAFR, MLD, MLDR, PL-HKR, PS-HKR, PSA, PSAR, PSS, PSSR, or both the CNF and UNF as specified are utilized; a microarray SGDS or DGDS type 2 direct label LPN assay, and the CNFs include one or more or all of C-HKR, spatial, print tip, print plate, intensity, scale, or the UNFs include one or more or all of A•SC, A•SCR, R•SC, R•SCR, PAF, PAFR, PL-HKR, PS-HKR, LLS, LLSR, or both the CNF and UNF as specified are utilized; a DGSS type 2 direct LPN assay, and the CNFs include one or more or all of C-HKR, spatial, print tip, print plate, intensity, scale, or the UNFs include one or more or all of A•SC, R•SC, PAF, PAFR, PL-HKR, PS-HKR, LLS, LLSR, or both the CNF and UNF as specified are utilized; a SGDS or DGDS type 1 indirect LPN assay, and the CNFs include one or more or all of C-HKR, spatial, print tip, print plate, intensity and scale, or the UNFs include one or more or all of A•SC, A•SCR, R•SC, R•SCR, PAF, PAFR, MLD, MLDR, PL-HKR, PS-HKR, SBN, SBNR, SSA, SSAR, or both the CNF and UNF as specified are utilized; a DGSS type 1 indirect LPN assay, and the CNFs include one or more or all of C-HKR, spatial, print tip, print plate, intensity, scale, or the UNFs include one or more or all of A•SC, R•SC, PAF, PAFR, MLD, MLDR, PL-HKR, PS-HKR, SBN, SBNR, SSA, SSAR, or both the CNF and UNF as specified are utilized; a SGDS or DGDS type 2 indirect LPN assay, and the CNFs include one or more or all of C-HKR, spatial, print tip, print plate, intensity, scale, or the UNFs include one or more or all of A•SC, A•SCR, R•SC, R•SCR, PAF, PAFR, PL-HKR, PS-HKR, SBN, SBNR, LLS, LLSR, or both the CNF and UNF as specified are utilized; and a DGSS type 2 indirect LPN assay, and the CNFs include one or more or all of C-HKR, spatial, print tip, print plate, intensity, scale, or the UNFs include one or more or all of A•SC, R•SC, PAF, PAFR, PL-HKR, PS-HKR, SBN, SBNR, LLS, LLSR, or both the CNF and UNF as specified are utilized.

In similar particular embodiments, assay is a non-microarray northern blot assay which analyzes cell sample RNA transcripts or equivalent cDNA or cRNA nucleic acids and which is a SGDS type 1 or type 2 direct LPN assay which analyzes cell sample RNA transcripts or equivalent cRNA nucleic acids, and the CNFs include one or more or all of C-HKR, spatial, intensity, or the UNFs include one or more or all of A•SC, A•SCR, R•SC, R•SCR, PAF, PAFR, or both the CNF and UNF as specified are utilized; a DGDS type 1 direct LPN assay, and one or more or all of the CNFs C-HKR, spatial, intensity, or one or more of the UNFs A•SC, A•SCR, R•SC, R•SCR, PAF, PAFR, MLD, MLDR, PL-HKR, PS-HKR, PSA, PSAR, PSS, PSSR, or both the CNF and UNF as specified are utilized; a DGDS type 1 indirect LPN assay, and the CNFs include one or more or all of C-HKR, spatial, intensity, or the UNFs include one or more or all of A•SC, A•SCR, R•SC, R•SCR, PAF, PAFR, MLD, MLDR, PL-HKR, PS-HKR, SBN, SBNR, SSA, SSAR, or both the CNF and UNF as specified are utilized; a DGDS type 2 direct LPN assay, and the CNFs include one or more or all of C-HKR, spatial, intensity, or the UNFs include one or more or all of A•SC, A•SCR, R•SC, R•SCR, PAF, PAFR, PL-HKR, PS-HKR, LLS, LLSR, or both the CNF and UNF as specified are utilized; a DGDS type 2 indirect LPN assay, and the CNFs include one or more or all of C-HKR, spatial, intensity, and UNFs include one or more or all of A•SC, A•SCR, R•SC, R•SCR, PAF, PAFR, PL-HKR, PS-HKR, SBN, SBNR, LLS, LLSR, or both the CNF and UNF as specified are utilized; a DGSS type 1 direct LPN assay, and the CNFs include one or more or all of C-HKR, spatial, intensity, or the UNFs include one or more or all of A•SC, A•SCR, R•SC, R•SCR, PAF, PAFR, MLD, MLDR, PL-HKR, PS-HKR, PSA, PSAR, PSS, PSSR, or both the CNF and UNF as specified are utilized; a DGSS type 1 indirect LPN assay, and the CNFs include one or more or all of C-HKR, spatial, intensity, or the UNFs include one or more or all of A•SC, A•SCR, R•SC, R•SCR, PAF, PAFR, MLD, MLDR, PL-HKR, PS-HKR, SBN, SBNR, SSA, SSAR, or both the CNF and UNF as specified are utilized; a DGSS type 2 direct LPN assay, and the CNFs include one or more or all of C-HKR, spatial, intensity, or the UNFs include one or more or all of A•SC, A•SCR, R•SC, R•SCR, PAF, PAFR, PL-HKR, PS-HKR, LLS, LLSR, or both the CNF and UNF as specified are utilized; or a DGSS type 2 indirect LPN assay, and the CNFs include one or more or all of C-HKR, spatial, intensity, or the UNFs include one or more or all of A•SC, A•SCR, R•SC, R•SCR, PAF, PAFR, PL-HKR, PS-HKR, SBN, SBNR, LLS, LLSR, or both the CNF and UNF as specified are utilized.

In other similar embodiments, the assay is a non-microarray dot blot assay which analyzes cell sample RNA transcripts or equivalent cDNA or cRNA nucleic acids and in which the assay is a SGDS type 1 direct or indirect LPN assay, and the CNFs include one or more or all of C-HKR, spatial, intensity, or the UNFs include one or more or all of A•SC, A•SCR, R•SC, R•SCR, PAF, MLD, MLDR, or both the CNF and UNF as specified are utilized; a SGDS type 2 direct or indirect LPN assay, and the CNFs include one or more or all of C-HKR, spatial, intensity, or the UNFs include one or more or all of A•SC, A•SCR, R•SC, R•SCR, PAF, PAFR, or both the CNF and UNF as specified are utilized; a DGDS type 1 direct LPN assay, and the CNFs include one or more or all of C-HKR, spatial, intensity, or the UNFs include one or more or all of A•SC, A•SCR, R•SC, R•SCR, PAF, PAFR, MLD, MLDR, PL-HKR, PS-HKR, PSA, PSAR, PSS, PSSR, or both the CNF and UNF as specified are utilized; a DGDS type 1 indirect LPN assay, and the CNFs include one or more or all of C-HKR, spatial, intensity, or the UNFs include one or more or all of A•SC, A•SCR, R•SC, R•SCR, PAF, PAFR, MLD, MLDR, PL-HKR, PS-HKR, SBN, SBNR, SSA, SSAR, or both the CNF and UNF as specified are utilized; a DGDS type 2 direct LPN assay, and the CNFs include one or more or all of C-HKR, spatial intensity, or the UNFs include one or more or all of A•SC, A•SCR, R•SC, R•SCR, PAF, PAFR, PL-HKR, PS-HKR, LLS, LLSR, or both the CNF and UNF as specified are utilized; a DGDS type 2 indirect LPN assay, and the CNFs include one or more or all of C-HKR, spatial, intensity, or the UNFs include one or more or all of A•SC, A•SCR, R•SC, R•SCR, PAF, PAFR, PL-HKR, PS-HKR, SBN, SBNR, LLS, LLSR, or both the CNF and UNF as specified are utilized; a DGSS type 1 direct LPN assay, and the CNFs include one or more or all of C-HKR, intensity, or the UNFs include one or more or all of A•SC, A•SCR, R•SC, R•SCR, PAF, PAFR, MLD, MLDR, PL-HKR, PS-HKR, PSA, PSAR, PSS, PSSR, or both the CNF and UNF as specified are utilized; a DGSS type 1 indirect LPN assay, and the CNFs include one or more or all of C-HKR, intensity, or the UNFs include one or more or all of A•SC, A•SCR, R•SC, R•SCR, PAF, PAFR, MLD, MLDR, PL-HKR, PS-HKR, SBN, SBNR, SSA, SSAR, or both the CNF and UNF as specified are utilized; a DGSS type 2 direct LPN assay, and the CNFs include one or more or all of C-HKR, intensity, or the UNFs include one or more or all of A•SC, A•SCR, R•SC, R•SCR, PAF, PAFR, PL-HKR, PS-HKR, SBN, SBNR, LLS, LLSR, or both the CNF and UNF as specified are utilized; or a DGSS type 2 indirect LPN assay, and the CNFs include one or more or all of C-HKR, intensity, or the UNFs include one or more or all of A•SC, A•SCR, R•SC, R•SCR, PAF, PAFR, PL-HKR, PS-HKR, SBN, SBNR, LLS, LLSR, or both the CNF and UNF as specified are utilized.

In still other similar embodiments, the assay is a non-microarray nuclease protection assay which analyzes cell sample RNA transcripts or equivalent cDNA or cRNA nucleic acids and which is a SGDS type 1 or type 2 direct or indirect LPN assay, and the CNFs include one or more or all of C-HKR, intensity, or the UNFs include one or more or all of A•SC, A•SCR, R•SC, R•SCR, PAF, PAFR, MLD, MLDR, or both the CNF and UNF as specified are utilized; a DGDS type 1 direct LPN assay, and the CNFs include one or more or all of C-HKR, intensity, or the UNFs include one or more or all of A•SC, A•SCR, R•SC, R•SCR, PAF, PAFR, MLD, MLDR, PL-HKR, PS-HKR, PSA, PSAR, PSS, PSSR, or both the CNF and UNF as specified are utilized; a DGDS type 2 direct LPN assay, and the CNFs include one or more or all of C-HKR, intensity, or the UNFs include one or more or all of A•SC, A•SCR, R•SC, R•SCR, PAF, PAFR, PL-HKR, PS-HKR, LLS, LLSR, or both the CNF and UNF as specified are utilized; a DGDS type 1 indirect LPN assay, and the CNFs include one or more or all of C-HKR, intensity, or the UNFs include one or more or all of A•SC, A•SCR, R•SC, R•SCR, PAF, PAFR, MLD, MLDR, PL-HKR, PS-HKR, SBN, SBNR, SSA, SSAR, or both the CNF and UNF as specified are utilized; a DGDS type 2 indirect LPN assay, and the CNFs include one or more or all of C-HKR, intensity, or the UNFs include one or more or all of A•SC, A•SCR, R•SC, R•SCR, PAF, PAFR, PL-HKR, PS-HKR, SBN, SBNR, LLS, LLSR, or both the CNF and UNF as specified are utilized; a DGSS type 1 direct LPN assay, and the CNFs include one or more or all of C-HKR, intensity, or the UNFs include one or more or all of A•SC, A•SCR, R•SC, R•SCR, PAF, PAFR, MLD, MLDR, PL-HKR, PS-HKR, PSA, PSAR, PSS, PSSR, or both the CNF and UNF as specified are utilized; a DGSS type 2 direct LPN assay, and the CNFs include one or more or all of C-HKR intensity, or the UNFs include one or more or all of A•SC, A•SCR, R•SC, R•SCR, PAF, PAFR, PL-HKR, PS-HKR, LLS, LLSR, or both the CNF and UNF as specified are utilized; a DGSS type 1 indirect LPN assay, and the CNFs include one or more or all of C-HKR, intensity, or the UNFs include one or more or all of A•SC, A•SCR, R•SC, R•SCR, PAF, PAFR, PL-HKR, PS-HKR, SBN, SBNR, SSA, SSAR, or both the CNF and UNF as specified are utilized; or a DGSS type 2 indirect LPN assay, and the CNFs include one or more or all of C-HKR, intensity, or the UNFs include one or more or all of A•SC, A•SCR, R•SC, R•SCR, PAF, PAFR, PL-HKR, PS-HKR, SBN, SBNR, LLS, LLSR, or both the CNF and UNF as specified are utilized.

In other similar embodiments, the assay is a non-microarray RT-PCR assay which analyzes cell sample RNA transcripts or equivalent cDNA or cRNA nucleic acids, and which is a SGDS, DGDS, or DGSS, assay, and the CNFs include one or more or all of AE•SE, AE•SER, AE•AE, AE•AER, or the UNFs include one or more or all of A•SC, A•SCR, R•SC, R•SCR, PAF, PAFR, or both the CNF and UNF as specified are utilized; or a SGDS, DGDS, or DGSS assay also analyzes one or more exogenous and/or endogenous standard RNA (S RNA) transcripts or equivalent cDNA or cRNA nucleic acids, and the CNFs include one or more or all of AE•SE, AE•SER, AE•AE, AE•AER, or the UNFs include one or more or all of A•SC, A•SCR, R•SC, R•SCR, PAF, PAFR, or both the CNF and UNF as specified are utilized.

In still further similar embodiments, the assay is a clone counting or SAGE method assay which analyzes cell sample RNA transcripts or equivalent cDNA or cRNA nucleic acids, which is a SGDS, DGDS, or DGSS, assay, and the CNFs comprise one or more or all of sampling statistics, sequencing error, or the UNFs comprise one or more or all of STM, STMR, PAF, PAFR, or both the CNF and UNF as specified are utilized; or a SGDS, DGDS, or DGSS, assay in which one or more exogenous or endogenous standard, RNA transcripts or equivalent cDNA or cRNA nucleic acids are analyzed, and the CNFs comprise one or more or all of sampling statistics and sequencing error, or the UNFs comprise one or more or all of STM, STMR, PAF, PAFR, or both the CNF and UNF as specified are utilized.

In further embodiments, the improved PG RNA transcript expression analysis assay results produced include one or more or all of the following:
(a) an assay measured and normalized relative or absolute value for the number of RNA transcripts per sample cell, for one or more or all of the different assay detectable PG RNA transcripts which are present in the analyzed cell sample RNA transcript preparation;
(b) a normalized differential gene expression ratio (N-DGER) value for a different gene same cell sample (DGSS), same gene different cell sample (SGDS), or different gene different cell sample (DGDS) RNA transcript expression analysis assay comparison of different particular gene RNA transcripts which are present in the same cell sample RNA transcript preparation;
(c) an assay measured and normalized relative or absolute value for the RN value for one or more or all of the different PG RNA transcripts which are present in an aliquot of a cell sample RNA transcript preparation; and
(f) a combination of one or more or all possible, SGDS, DGDS, and/or DGSS particular gene RNA transcript comparison N-DGER values, and PG relative or absolute RN or abundance values, from one or more different RNA transcript expression analysis assays.

In still further embodiments, the gene expression RNA transcript expression analysis assay of a cell sample RNA transcript preparation or equivalent cDNA or cRNA nucleic acids, utilizes one or more exogenous RNA or DNA transcript artificial housekeeping gene standards or one or more valid endogenous RNA transcript true housekeeping gene standards, to produce for one or more non-housekeeping PGs in the assay one, each combination of two, or all three of
(a) improved relative or absolute values or both for a PG abundance or number of RNA transcripts per sample cell which is present in the analyzed cell sample,
(b) improved relative or absolute values or both for the number of PG RNA transcripts per sample cell haploid DNA content; and
(c) improved relative or absolute values or both for a PG RN which is associated with an aliquot of analyzed cell sample RNA.

In certain embodiments, such AHGs (which may be in combination with valid endogenous RNA transcript true housekeeping gene standards) are used to facilitate the determination of assay pertinent UNF and CNF values, by
a) determining the number of each cell sample's cell equivalents (CE) present in the cell sample nucleic acid sample being analyzed in the assay;
b) adding a known number of molecules for each of one or more particular RNA or DNA standards to each cell sample nucleic acid sample being analyzed in the assay, thereby producing in each cell sample nucleic acid sample being analyzed in the assay one or more artificial housekeeping gene (AHG) particular RNAs or DNAs whose copy per cell or abundance value is known;
c) performing the assay and producing raw assay results for each particular cell sample particular gene and particular AHG; and
d) utilizing the raw assay results for at least one particular standard AHG and the known abundance value for the particular standard AHG in the sample and the known true differential gene expression ratio value for the particular standard AHG in compared cell samples in determining the assay values for UNFs and/or CNFs which are pertinent for the assay; such UNFs and/or CNFs can then be used to normalize the particular gene assay results.

In embodiments in which AHGs are used, one or a plurality of AHGs are used, e.g., at least 2, 3, 4, 5, 6, 7, 8, 10, 20, 50, 100, 200, 500, 1000, or even more AHGs; the number of RNA or DNA molecules added to each nucleic acid sample differs for two or more different AHG standards (e.g., for 2, 3, 4, 5, 10, 20, or even more different AHG standards); in a plurality of different AHG standards the different AHG standards differ in at least one (or each combination taken 2, 3, or 4 at a time, or all 5) of the characteristics: a) nucleotide sequence, b) the nucleotide length, c) the nucleotide composition, d) the nucleotide sequence secondary structure, and e) the direct or indirect label density; at least one particular RNA or DNA AHG molecule (or a greater number, e.g., as specified above for the numbers added) is directly or indirectly prelabeled before addition; such prelabeling can be to a known quantitive degree or to an unknown quantitative degree before addition.

In particular embodiments, such AHGs and/or endogenous true housekeeping genes are applicable to any of a variety of assay types, for example, a) a microarray assay, b) a DOT blot assay, c) a northern blot assay, d) a nuclease protection assay, e) an RT-PCR assay, or f) a clone counting or SAGE assay.

Any source or type of cells, or type of transcript preparation can be analyzed with improved results. Thus, in particular embodiments of the present method, the cell sample RNA transcripts or equivalents targeted and analyzed include unspliced and unprocessed, partially processed and processed, or completely spliced and processed, cell sample associated RNA transcripts; the cell sample RNA transcript preparation analyzed or the cell sample RNA transcript preparations compared are derived from one or more of the following sources:
(a) one or more prokaryotic cell samples which are derived from cultured or naturally occurring prokaryotic organisms, or
(b) one or more prokaryotic cell samples infected with a virus or with another prokaryotic cell, or
(c) one or more prokaryotic cell samples of the same prokaryotic series or strain or other classification, or
(d) one or more prokaryotic cell samples of a different prokaryotic species or strain or other classification, or
(e) one or more prokaryotic cell samples which have been exposed one or a set of particular environmental conditions, such as light (e.g., UV light), radioactivity, a physical condition (e.g., pressure), chemical exposure, particular nutritional conditions, drug exposure (e.g., anti-bacterial agent or drug being tested for such activity), or other stimulus or treatment, or
(f) one or more prokaryotic cell samples of the same strain or species which are in different growth or nutritional states, or
(g) any other known or unknown cultured or natural prokaryotic cell sample or mixtures of cell samples of different types, or
(h) any combination of two or more of items a-g, or
(i) one or more eukaryotic cell samples which are derived from cultured or naturally occurring eukaryotic cells, tissues, or organisms, or
(j) one or more eukaryotic cell samples infected with a virus or with a virus and/or a prokaryotic cell and/or another eukaryotic cell, or
(k) one or more cell samples of the same eukaryotic species or strain or
(l) one or more cell samples of the same eukaryotic species or strain and the same or different state of growth and/or nutrition, or
(m) one or more cell samples of the same eukaryotic species or strain and the same or different state of differentiation and/or growth and/or nutrition, or
(n) one or more normal or diseased or pathologic cell samples of the same eukaryotic species or strain which have been treated with the same or different physical or chemical stimuli or other treatment (e.g., as indicated for projaryotic cells above), or
(o) one or more cell samples of primary or continuous culture eukaryotic cell samples of the same or different cell type and species, or strain, or
(p) one or more cell samples of primary or continuous culture eukaryotic cell samples of the same or different state of growth or nutrition, or
(q) one or more cell samples of primary or continuous culture eukaryotic cell samples which have the same or different states of differentiation, or
(r) one or more normal or diseased or pathologic eukaryotic tissue cell samples from the same or different eukaryotic organisms which are at the same or different states of differentiation, growth, and nutrition, or
(s) one or more eukaryotic tissue cell samples from a eukaryotic organism which have been treated with the same or different physical and/or chemical and/or other stimuli, or
(t) one or more primary or continuous culture eukaryotic organism tissue, or
(u) one or more cultured or natural eukaryotic cell sample or tissue or organism type or mixtures of such cell samples, or
(v) one or more cultured or natural eukaryotic cell sample, or tissue, or organisms, which are infected with a virus, a prokaryote cell or another eukaryotic cell type, or
(w) any other known or unknown cultured or natural eukaryotic cells or cell types, tissues or tissue types, or organisms or organism types, or
(x) any possible combination of items (i) through (w)
(y) any possible combination of items (a) through (x).
For any of embodiments above (e.g., for any of the cell sample sources and types), the sample can be of various content characteristics. Thus, in further embodiments, the cell sample RNA transcripts or equivalents targeted and analyzed include unspliced and unprocessed, unspliced and partially processed, and and/or unspliced and processed, and/or partially spliced and partically processed, and/or completely spliced and processed, cell sample associated RNA transcripts; such analyzed cell sample RNA transcripts or equivalent nucleic acids derived therefrom represent one or more of:
(a) cell sample total RNA transcripts, or
(b) cell sample isolated mRNA transcripts, or
(c) one or more cell sample PG mRNA transcripts which are present in total RNA or isolated mRNA, or
(d) cell sample total PG mRNA transcripts, or
(e) cell sample isolated PG mRNA transcripts, or
(f) one or more cell sample PG mRNA transcripts which are present in total RNA or isolated miRNA, or
(g) cell sample total PG siRNA transcripts, or
(h) cell sample isolated PG siRNA transcripts, or
(i) one or more cell sample PG siRNA transcripts which are present in total RNA or isolated siRNA, or
(j) cell sample total PG snoRNA transcripts, or
(k) cell sample isolated PG snoRNA transcripts, or
(l) one or more cell sample PG snoRNA transcripts which are present in total RNA or isolated RNA, or
(m) cell sample total PG rRNA transcripts, or
(n) cell sample isolated PG rRNA transcripts, or
(o) one or more cell sample PG rRNA transcripts which are present in total RNA or isolated RNA, or
(p) cell sample total PG tRNA transcripts, or
(q) cell sample isolated PG tRNA transcript, or
(r) one or more cell sample PG tRNA transcripts which are present in total RNA or isolated RNA, or
(s) one or more virus PG RNAs or virus PG RNA transcripts produced from virus RNA or DNA genes which are present in a cell sample total RNA or a cell sample isolated RNA, or
(t) foreign prokaryotic or eukaryotic cell total RNA, mRNA, miRNA, siRNA, snoRNA, rRNA, or tRNA transcripts or combinations thereof which are present in a cell sample total RNA or isolated RNA preparation, or
(u) one or more endogenous RNA transcripts which are present in cell sample total RNA or isolated RNA, or
(v) one or more exogenous RNA transcripts which are present in cell sample total RNA or isolated RNA.
In additional embodiments, the cell sample gene expression analysis assay of one or more cell sample RNA transcript preparations or equivalent nucleic acids derived therefrom, incorporates one or more of the following assay design solutions,
(a) as few assay pertinent UNFs as possible;
(b) as many assay pertinent UNF assay values as possible equal one;
(c) as few CNFs as possible are assay pertinent;
(d) as many assay pertinent CNF assay values as possible equal one;
(e) the occurrence of CNF and UNF related false negative particular gene assay results is minimized or eliminated;
(f) the use in the assay of one or more exogenous standard artificial housekeeping gene (AHG) RNAs or DNAs in order to simplify and improve the determination of the assay values for one or more assay pertinent CNFs or one or more assay pertinent UNFs or both;
(g) the use in the assay of one or more exogenous standard RNAs or DNAs in order to simplify and improve the determination of the assay values for one or more assay pertinent CNFs or one or more assay pertinent UNFs or both;
(h) the identification of and the use in the assay of one or more true housekeeping gene RNA transcripts which are endogenous to the cell sample or cell samples, in order to simplify and improve the determination of the assay values for one or more assay pertinent CNFs or one or more assay pertinent UNFs or both; and
(i) the use of one or more AHG or true housekeeping gene or both RNA or DNA transcripts whose abundance values are known, in order to determine the abundance values of one or more non-control PG RNA transcripts in a cell sample.

In still further embodiments, for each particular gene RNA transcript comparison or particular gene RNA transcript equivalent cDNA or cRNA comparison in the assay, the A•SCR assay value is used to measure the particular gene comparison assay result in terms of gene RNA copies per sample cell or the R•SCR assay value is used to measure the particular gene comparison in terms of gene RNA copies per haploid cell DNA content, or both; the A•SCR assay value is used to measure the particular gene comparison assay result in terms of RNA copies per sample cell; the R•SCR assay value is used to measure the particular gene comparison in terms of gene activity per haploid cell DNA content.

In yet further embodiments and related aspects, design solutions as specified in the design solution tables herein are utilized for producing improved assay measured SGDS, DGDS, or DGSS particular gene RNA transcript expression comparison N-DGER values which are known to be improved in normalization and interpretation relative to corresponding prior art assay produced gene expression comparison N-DGER values, e.g., in a method using a microarray assay, a design solution combination is utilized in the assay where (a) the design solution combination is selected from the group consisting of the design solution combinations presented in Tables 54-60, 75-81, and 100-102; or (b) the design solution combination is selected from the group consisting of the design solution combinations presented in Tables 61-69, and 82-90; in a method using a northern blot assay a design solution combination selected from the group of design solution combinations presented in Table 93 is utilized; in a method using a dot blot assay a design solution combination selected from the group of design solution combinations presented in Table 94 is utilized; in a method using a nuclease protection assay a design solution combination selected from the group consisting of the design solution combinations presented in Table 95 is utilized; in a method using a RT-PCR assay a design solution selected from the group consisting of the design solution combinations presented in Table 97 is utilized; in a method using a clone counting method assay a sign solution selected from the group consisting of the design solution combinations presented in Table 99 is utilized.

For the aspect and embodiments above, in particular aspect, the particular cell sample RNA transcript type analyzed in the assay includes one or more or all of different particular precursor and mature RNA transcript types which are present in the compared cell sample total RNA transcripts preparations; the transcripts include the RNA transcripts of all types which are present in a cell sample total RNA transcript preparation; the transcripts include one or more of:
(a) mRNA transcripts of one or more or all types;
(b) rRNA transcripts of one or more or all types;
(c) tRNA transcripts of one or more or all types;
(d) siRNA transcripts of one or more or all types;
(e) miRNA transcripts of one or more or all types;
(f) snoRNA transcripts of one or more or all types;
(g) regulatory RNA transcripts of one or more or all types;
(h) any other RNA transcripts of one or more or all types; and
(i) one or more combinations of two or more or all of the above described RNA transcript types.

Another set of related aspects of the present invention concerns assay kits for improving, validating, calibrating, and/or corroborating a particular gene (PG) RNA transcript expression analysis assay or PG transcript comparison analysis or both for a cell sample RNA transcript preparation or equivalent nucleic acids derived therefrom. In such aspects, the assay kit includes a set of components (which may be packaged). In one such aspect, the assay kit includes a reagent set (e.g., packaged or otherwise assembled or collected together) including at least one reagent for carrying out the assay, and either or both of instructions for performing the assay with improved normalization (e.g., according to the methods described above or otherwise described herein), or a quantity of at least one improved normalization reagent for obtaining one or more of the improved normalization, validation, calibration, and corroboration.

In particular embodiments, the assay kit includes the instructions for performing the assay with improved normalization and not the improved normalization reagent, or the improved normalization reagent and not the instructions; the normalization reagent includes at least one defined RNA or DNA (or a greater number as described above); the at least one defined RNA or DNA is or includes at least one artificial housingkeeping gene (AHG) (e.g., where use of the AHG improves determination of one or more assay pertinent UNFs or CNFs or both); the assay kit includes both the instructions and the at least one AHG; the improved normalization reagent includes a quantity of at least one cell sample total RNA or isolated mRNA for which is known characteristic data (which may be included in the assay kit or available separately), e.g., selected from the group consisting of a) the mass amount of cell sample total RNA per cell, b) the mass amount of cell sample mRNA per cell, c) the number of mRNA transcripts of any kind per cell, for each particular RNA sample, d) both a) and b), e) both a) and c), f) both b) and c), g) all of a) and b) and c); the number of PG RNA molecules per cell is also known for one or more PGs in the cell sample; the assay kit includes a quantity of at least one cell sample cDNA LPN or cRNA LPN or both, for which is known one or more of the characteristic data: a) the mass amount of cell sample cDNA LPN or cRNA LPN per cell equivalent (CE) or both, b) the number of cDNA or cRNA transcripts per CE for one or more PG cDNAs or PG cRNAS or both which are present in the cell sample cDNA or cRNA preparation; instructions and/or the characteristic data may be provided in the assay kit.

In certain embodiments, the improved normalization reagent includes one or more reagents for determining quantitative values for any 1, 2, 3, 4, or 5 of a) the mass of total DNA per intact cell, b) the total mass of DNA present in the intact cell sample aliquot which is analyzed in the assay, c) a cell sample's mass amount of total RNA per intact cell or mRNA per intact cell or both, d) the number of mRNA transcripts per intact cell, and e) the number of RNA molecules per cell in the cell sample for one or more PGs, instructions may be included in the kit, which may include directions for determining the quantitative values.

Similarly, in certain embodiments, the improved normalization reagent includes reagents for determining quantitative values for one or more of the following a) the mass amount of total cell sample cDNA LPN or cell sample cRNA LPN per intact cell or both, for each cell sample of interest, b) the mass amount of total cell sample cDNA LPN or cRNA LPN or both which is analysed in an assay, c) the number of cell sample cDNA or cRNA cell equivalents (CE) which are analysed in an assay, d) the cDNA or cRNA associated sample cell number (SC) value or both, for each assayed cell sample, e) the cell sample comparison cDNA or cRNA SCR value or both for each cll sample assay comparison, and f) the number of cDNA or cRNA transcripts per CE for one or more PGs in the cell sample cDNA or cRNA preparation or both, instructions and/or directions for determining those quantitative values may be included in the assay kit.

In particular embodiments, the improved normalization reagent includes a quantity of at least one of: a) one or more RNA or DNA oligonucleotides which are improved characterized RNA or DNA, or improved synthesis RNA or DNA, or both, b) modified RNA or DNA oligonucleotide which may be improved synthesis, c) RNA or DNA analog oligonucleotide which may be improved synthesis; such oligonucleotide or oligonucleotide analog is associated with or used for normalization improvement for the assay; the kit includes the instructions. In general, such oligonucleotides (that is un-modified and modified nucleotides and nucleotide analogs) are improved in characterization or synthesis or both

Also in certain embodiments, the improved normalization reagent includes one or more reagents for isolating RNA or DNA or both from a cell sample and determining quantitative values for one or more of: a) the cell sample's mass amount of total RNA per intact cell, b) the cell sample's mass amount of mRNA per intact cell, c) the cell sample's mass amount of total DNA per intact cell, d) the mass amount of DNA present in the intact cell sample aliquot which is analysed in the assay, and the number of mRNA transcripts per intact cell for the cell sample; the kit also includes instructions, e.g., for determining such quantititative values.

In particular embodiments, the reagent set includes at least one microarray (e.g., a cDNA microarray); a reverse transcriptase selected as suitable for performing RT-PCR; heat stable DNA polymerase selected as suitable for performing PCR; at least one oligonucleotide primer suitable for priming enzymatic reverse transcriptase mediated or DNA or RNA polymerase mediated in vitro enzymatic synthesis, or both, of cell sample-derived nucleic acid; one or more nucleases selected as suitable for performing a nuclease protection assay.

In some embodiments, the assay kit includes one or more reagents for validating a microarray or RT-PCR assay result by an independent gene expression analysis method (and may include instructions); the independent gene expression analysis method comprises one or more of: a nuclease protection assay, a hydroxyapatite assay, an ELISA assay, an affinity column separation assay, and a centrifugation separation assay.

In particular embodiments, the assay kit includes reagents for producing cell sample enzymatically synthesized directly or indirectly labeled polynucleotide (LPN) preparations to be used for gene expression comparison analysis assays, where the average nucleotide length of the newly synthesized LPN prep molecules is the same or nearly the same for each produced and compared LPN preparation, e.g., the average nucleotide lengths of the compared LPN preparations differ by less than 4, 3, 2, 1.5, 1.25, or 1.1 fold; the kit also includes the instructions.

Likewise in particular embodiments, the assay kit includes reagents for determining the average nucleotide length of one or more PG LPN populations in one or more cell sample LPN preparations, and may include the instructions; the reagent set includes quantities of labeled nucleotides or nucleotide analogs; the reagent set comprises a quantity of un-labeled nucleotides or nucleotide analogs.

In particular embodiments, the assay kit includes a system which is or includes one or more of the following: a) an oligonucleotide microarray system, b) an oligonucleotide (e.g., cDNA) microarray system, c) a clone counting or SAGE system, d) a nuclease protection assay system, e) a RT-PCR system; or f) a gene expression analysis system; the system is a commercial or homebrew system; such commercial or homebrew system is or includes one or more of the types of systems just indicated; a commercial system is or includes an AFFYMETRIX system, a GE HEALTHCARE system, an AGILENT system, a COMBIMATRIX system, an OXFORD GENE TECHNOLOGY SYSTEM, a NIMBLEGEN system, a FEBIT system, a CLONTECH system, a GENOSPECTRA system, a HIGH THROUGHPUT GENOMICS system, a SOLEXA system, an ABI microarray system, an ABI RT-PCR system, or a system from a successor of an identified entity.

In addition, assay kits can be supplied for providing information useful in improving, validating, calibrating, or corroborating another assay process and/or results of such other assay. Thus, another aspect concerns an assay kit for improving, validating, calibrating, or corroborating a PG RNA transcript gene expression analysis result or gene expression comparison analysis result for a particular cell sample, where the assaykit includes a quantity of at least one purified particular cell sample total RNA (T-RNA) preparation or a purified cell sample mRNA preparation or both, for which is known for the cell sample one or more or all of the following preparation parameters: a) the mass of cell sample T-RNA per intact cell, b) the mass amount of cell sample total mRNA per cell, c) the number of mRNA transcripts per intact cell, and d) the mass of DNA per intact cell; the kit can also include instructions for using the T-RNA preparation or mRNA preparation to provide improved normalization, validation, calibration, or corroboration for a PG RNA transcript gene expression analysis result or gene expression comparison analysis result for a particular cell sample, and/or preparation parameter data; the preparation parameter, the number of PG RNA molecules per cell for the cell sample, is also known, and may be specified for one or more particular genes in the cell sample.

Similarly, another aspect concerns an assay kit for improving or validating or calibrating or corroborating a PG RNA transcript gene expression analysis result or gene expression comparison analysis result for a particular cell sample, which includes a quantity of at least one purified particular cell sample cDNA LPN preparation or a cRNA LPN preparation or both, for which the mass of cell sample cDNA LPN or cRNA LPN per intact cell or both is known.

In certain embodiments, mass of cell sample cDNA LPN or cRNA LPN per intact cell or both is specified in said assay kit; the number of PG cDNA or cRNA transcripts per CE for one or more PGs which are present in the cell sample cDNA or cRNA preparations or both is also known, and may be specified in the assay kit; the number of PG cDNA or cRNA transcripts per CE for one or more PGs is known, and may be specified in the assay kit; the assay kit includes the instructions.

In addition the methods and assay kits described above, computer implementation of at least portions of the present method are highly useful. Thus, one such aspect concerns a computer accessible database which contains at least one data set stored in a computer accessible electronic storage medium configured for use in execution of software for providing improved normalization of results from a gene expression assay or a gene expression comparison assay or both. Thus, in particular embodimens, the database contains any of the types of data indicated herein as useful for performing improved normalization of such assay results. For example, in particular embodiments, the database contains one or a plurality of data sets from the following list (e.g., at least 2, 3, 4 5, 6, 7, 8, 9, or 10 of the exemplary categories of data indicated):
nucleotide sequence or sequence related data or both for the RNA of interest from a particular cell type; such sequence related data can include, for example, length, composition, and secondary structure;
sequence or sequence related data (e.g., as indicated above) or both for RNA from a plurality of different types of cells;
data describing one or more characteristics for variant or processed forms of particular genes and RNAs;
data describing at least one of the nucleotide sequence (NS), nucleotide length (NL), and nucleotide sequence composition (NC) of one or more (e.g., a set) of nucleic acid capture or detection probes;
data describing the effect of some or all of the length, sequence, composition, and secondary structure of the nucleic acid target or probe molecule(s) or both on the kinetics or completeness of hybridization or both of particular gene target (PG-T) molecules with a complementary nucleic acid capture probe or other complementary nucleic acid molecule or both;
data describing the effects of one or more of the label density, label location, and label type of a PG-T on the kinetics or completeness of hybridization or both of the target with a complementary oligonucleotide;
data describing the effect of label density on the magnitude of the signal intensity associated with the target, e.g., under assay conditions;
data describing the relationships between the sample target labeling conditions and compositions, and the efficiency of label molecule incorporation in different PG-T molecules;
data describing the relationship between the quantity of PG-T molecules measured under assay conditions and the intensity of signal obtained; and
data describing or characterizing the relationship between the average nucleotide length of a samples total target RNA or cDNA or cRNA molecules, and the average nucleotide length of particular gene (PG) RNA, cDNA, or cRNA molecule populations which are present in respective sample pools.

In particular embodiments, the data set is loaded in volatile memory or in non-volatile memory of a computer; the data set is embedded in a portable data storage device (e.g., a flash memory device, a CD, a DVD, or the like); the data set is embedded in a magnetic hard drive(s) of a computer or network; the data base is accessible from a stand alone computer, over a local area network (LAN), over a wide area network (WAN), over the internet.

A related aspect concerns a computer software program, usually stored in a computer accessible electronic storage medium, which includes a computer instruction set for providing improved normalization of assay results, e.g., for performing any of the calculations involved in the improved normalization described herein.

In certain embodiments, the instruction set includes instructions for calculating one or more improved UNF values selected from the group consisting of SCR, STMR, PAFR, MLDR, PL-HKR, PS-HKR, PSAR, PSSR, LLNR, LLSR, SBNR, and SSAR; the instruction set includes instructions for calculating one or more improved CNF values selected from the group consisting of spatial, print tip, print plate, intensity, and scale; the instruction set includes instructions for improved normalizing of assay results utilizing at least one improved normalization factor selected from the group consisting of SCR, STMR, PAFR, MLDR, PL-HKR, PS-HKR, PSAR, PSSR, LLNR, LLSR, SBNR, and SSAR; the instruction set includes instructions for improved normalizing of assay results utilizing at least one improved normalization factor selected from the group consisting of spatial, print tip, print plate, intensity, and scale; the instruction set includes instructions for performing calculations to determine one or more (e.g., any combination of 2, 3, 4, 5, 6, 7, 8, 9, 10, 11, or 12) of the following:
(i) the average nucleotide length for a PG-T molecule population in a sample target preparation;
(ii) the average NS, NC, and SS for a PG-T molecule population in a sample target preparation;
(iii) the label density (LD) for a PG-T molecule population in a sample target preparation;
(iv) the average mass of a PG-T nucleic acid which can hybridize to one spot immobilized complementary capture probe molecule;
(v) the effect of one or more of the NL, NS, NC, SS, and LD on the kinetics and completeness of hybridization of PG-T molecules to spot immobilized complementary capture probes or other complementary probes for a sample target preparation;
(vi) the effect of the PG-T LD value on the signal intensity produced by the PG-T for a PG-T in a sample target preparation;
(vii) the number of cell equivalents (CE) of sample target RNA, cDNA, or cRNA which are analyzed in the assay hybridization solution;
(viii) the proportionality of the relationship between the assay input RNA, dDNA, or cRNA concentration and the assay measured signal activity for spot hybridized PG-T molecules.
(ix) replicate sample or standard assay results or both;
(x) a data set specifying the spatial position of each PG capture probe on a micro array;
(xi) assay signal results for replicate assay results which represent known greatly different concentration inputs of standard RNA, cDNA, or cRNA into the assay;
(xii) a data set specifying the microtiter well origin of each replicate sample or standard microarray capture probe spot.

Another related aspect concerns a method for performing an improved normalization of gene expression assay results by using a computer loaded with a software program (e.g., as described for the preceding aspect) for performing improved normalization of gene expression assay results to validly normalize results for at least one gene expression assay or gene expression comparison assay.

In particular embodiments, the method includes performing any of the functions described for the software aspect above; the normalization includes improved normalizing of the assay results for one or more UNFs, e.g., including one or more UNFs selected from the group consisting of SCR, STMR, PAFR, MLDR, PL-HKR, PS-HKR, PSAR, PSSR, LLNR, LLSR, SPNR, and SSAR; the normalization includes improved normalizing of the assay results for one or more CNFs, e.g., including one or more CNFs selected from the group consisting of spatial, print tip, print plate, intensity, and scale; the normalization includes improved normalizing for one or more UNFs and one or more CNFs, e.g., as specified for the UNFs and CNFs individually.

The invention is particularly well adapted for use in developing improved gene expression assays and/or gene expression comparison assays, and corresponding assay kits and methods, or in improving existing such assays, kits, and methods. Thus, another aspect concerns a method for evaluating the performance of a gene expression analysis assay, where the method involves:
identifying the pertinent UNFs and CNFs which are associated with the assay;
identifying the normalization assumptions necessary for the valid normalization of assay pertinent CNF values by prior art methods;
determining the assay values for the pertinent UNFs;
determining the assay pertinent CNF values;
normalizing the cell sample and standard PG raw assay results for the determined pertinent UNF and CNF values;
determining quantitative assay metric values for the assay results; and
compare the resulting quantitative assay metric values for the assay with quantititative assay metric values for one or more different assays or one or more standards to evaluate the performance of the assay.

In certain embodimens, assay values for pertinent UNFs and/or assay pertinent CNFs are determined by improved normalization methods (e.g., as described herein); assay pertinent CNF values are determined by both prior art methods and by correlation with particular assay design; improved normalization is utilized to normalize the assay results for pertinent UNFs or to validly normalize the assay results for pertinent CNFs, or both.

In some embodiments, the method also includes obtaining or developing nucleic acid test materials which include cell sample and standard nucleic acid test materials which assist in providing improved UNF and CNF normalization of assay results; the method also includes developing test system quantitative assay metrics which can be used to quantitatively evaluate the performance of the assay done using the analysis system.

In particular embodiments, replicate results are produced for one or more standard or particular gene nucleic acids or both in a single assay run, or for results from a plurality of assay runs, or both, for one or more different assay conditions; the evaluation is performed for a plurality of different assays, e.g., at least 2, 3, 4, 5, 10, or more different assays (e.g., modifications.

In particular embodiments, the nucleic acid test materials include one or more of unlabeled standard RNA or DNA or both, unlabeled cell sample RNA or DNA or both, labeled standard RNA or DNA or both, labeled cell sample RNA or DNA or both, unlabeled standard cDNA, labeled standard cDNA, unlabeled cell sample cRNA; and labeled cell sample cRNA; the standard RNA or DNA is or includes artificial housekeeping genes (AHG); AHGs are of predetermined nucleotide length, sequence, composition, and/or degree of labeling; a plurality of different AHGs are used (e.g., at least 2, 3, 4, 5, 6, 7, 8, 10, 15, 20, 50, 100, or even more); the nucleic acid test materials includes one or more cell sample RNA or DNA or both, or cell sample cDNA or cRNA preparations or both, for which the mass of cell sample total RNA (T-RNA) or mRNA or cDNA or cRNA per intact cell is known and for each cell sample preparation the number of CEs analysed in an assay is known.

In certain embodiments, the quantitative assay metrics include one or more of a) linear dynamic range of detection of standard and PG RNA, cDNA or CRNA, b) standard or PG abundance values or both, c) standard or PG N-DGER values or both, d) limit of detection of PG RNA, cDNA, and cRNA, e) linearity of proportionality of standard or PG assay input RNA, cDNA, or cRNA concentrations and the observed assay signal, f) precision and reproducibility of assay replicate results, g) accuracy of replicate results, and g) detection specificity of standard and PG target RNA, cDNA, and cRNA.

The evaluation methods can readily be used in the process of developing or producing improved assay kits or systems. Thus, a related aspect concerns a method for producing an improved assay kit or assay analysis system, which includes utilizing an evaluation method as described for the preceding aspect to evaluate the performance of one or more gene expression or gene expression comparison analysis systems or assay kits of interest (reference or standard systems and/or kits may be included), identifying a kit or system having desired quantitative assay or system metrics, and making the identified kit or system.

In particular embodiments, the method includes using the above-described evaluation methods to evaluate the performance of a kit or system which has been modified in at least one respect from a prior configuration, comparing the performance results of the modified and unmodified kit or system to identify desirable modifications which improve the performance of said kit or system, and incorporating one or more of the identified desired modifications into the kit or system to provide an improved kit or system.

The ability to provide improved gene expression and/or gene expression comparison assay results provides additional improved results in methods which utilize the improved assay results. Thus, a further aspect concerns a method for producing improved application results, by utilizing improved assay results produced by any of the methods described herein for providing improved assay results in a an application to produce improved first order application results, such as improved results of one or more of the following applications:
(a) a data analysis and data mining analysis method;
(b) a gene expression profile measurement and identification method for normal, pathologic, or diseased cell samples and combinations thereof;
(c) a bioactive and pharmaceutical candidate or biomarker identification and discovery method;
(d) a systems biology analysis method;
(e) a toxic compound identification and discovery method;
(f) a method for developing gene expression based diagnostic test methods; and
(g) a quality assurance and quality control method for a gene expression analysis application or a method for discovery and identification of toxic compounds, drugs, or bioactive molecules, or combinations thereof.

In a similar aspect, the invention provides a method for producing improved second order application results, which involves utilizing improved first order application results produced by the method of the preceding aspect in a second order application.

In particular embodiments, the second order application is or includes an application selected from the following group: (a) a systems biology analysis method which uses improved data mining analysis results; (b) a gene regulatory discovery pathway method which uses improved data mining analysis and/or systems biology results; (c) a pharmaceutical or bioactive candidate or biomarker evaluation method using one or more of improved data mining analysis, systems biology analysis, toxicology analysis, and safety analysis results; (d) a method for producing improved pharmaceutical candidate development and biomarker discovery results using improved results from diagnostic tests, data mining analysis, toxicology analysis, systems biology analysis, gene regulatory pathway analysis, or QA/QC procedures, or combinations thereof; (e) a disease related gene expression profile based diagnostic method using one or more of improved data mining analysis, systems biology analysis, diagnostic test analysis, biomarker discovery, gene regulatory pathway analysis, and QA/QC procedures; (f) a method for producing improved toxicology or safety evaluation results or both for bioactive compounds by using improved results from one or more of data mining analysis, systems biology analysis, diagnostic test analysis, biomarker discovery, gene regulatory pathway analysis, and QA/QC procedures.

Yet another similar aspect concerns a method for producing improved results for a higher order application which directly or indirectly utilizes one or more gene expression assay abundance or RNA transcript number (RN) or normalized assay signal (NAS) results, or one or more gene expression comparison assay NASR or N-DGER results, where the method involves a) conducting one or more gene expression assays or one or more gene expression comparison assays or both; b) utilizing the methods of any of claims 1-195 to produce one or more improved application results (IRs) selected from the group consisting of improved gene expression assay abundance results, RN results, NAS results, gene expression comparison assay NASR results, and N-DGER results; and c) directly utilizing one or more IRs in a higher order application which directly utilizes gene expression assay or gene expression comparison assay results to produce higher order IRs.

In certain embodiments, the method further involves directly utilizing one or more of the improved higher order IRs in a different higher order application to produce different higher order IRs; the method can further involve a) directly utilizing one or more of the different higher order IRs in a still different higher order application to produce still different higher order IRs; and b) optionally utilizing IRs from progressively higher order applications which utilize other improved higher order application results.

In particular embodiments, the higher order application includes one or more of the following: a) a linear discriminant method; b) a K-nearest neighbor method; c) a neural network method; d) a decision tree method; e) a partially supervised method or supervised method or unsupervised method; f) a class discovery method; g) a time analysis series; h) a hierarchical agglomerative clustering method; i) a hierarchical divisive clustering method; j) a non-hierarchical K-means method; k) a self organizing maps and trees method; 1) a principal component analysis method or a relationship between clustering and principal component analysis method; m) a gene shaving method,

n) a clustering in discretised space method; o) a graph based clustering method; p) a Bayesian or model based clustering method and fussy clustering method; q) a clustering of genes and samples method; r) a combination of two or more methods (a)-(q); s) a drug or bioactive compound candidate validation application; t) a biomarker candidate discovery and validation application; u) a drug or bioactive compound candidate development and optimization application; v) a data mining analysis application; w) a systems biology analysis application; x) a drug candidate or bioactive compound candidate discovery process application; y) a drug candidate or bioactive compound candidate validation process application; z) a drug or bioactive compound candidate development and optimization process application; aa) a drug or bioactive compound candidate toxicology evaluation process application; bb) a biomarker discovery process application; cc) a drug or bioactive compound candidate manufacturing process application; dd) a drug or bioactive compound candidate QC/QA process application; ee) an application process for identifying and characterizing one or more of the following: one or more expressed genes, one or more gene expression profiles which are characteristic of a particular normal or diseased or pathologic cell sample, a particular cell sample treated with a particular drug or bioactive compound, or physical, chemical, or psychological treatment; ff) a regulatory pathway identification and/or analysis and/or monitoring process application; gg) a drug or bioactive compound candidate efficacy evaluation process application; hh) a drug or bioactive compounds selection process for clinical study patients application; ii) a drug or bioactive compound clinical trial monitoring process application; jj) a drug or bioactive compound market segment identification process application; kk) a drug or bioactive compound prescription to the patient or end user process application; ll) a drug or bioactive compounds effectiveness and/or safety in the patient process application; mm) a disease or pathologic status evaluation process process application; nn) a disease prognosis evaluation and monitoring before and after drug treatment process application; oo) a systems biology analysis application; pp) a drug or bioactive compound related diagnostic test development and use process application; qq) a process for monitoring long and short term drug and/or bioactive molecule effectiveness in the treated patient application; and rr) a process for monitoring the long and short term drug and/or bioactive molecule toxicity characteristics in the treated patient application.

Additional embodiments will be apparent from the Detailed Description and from the claims.

### DETAILED DESCRIPTION OF THE PREFERRED EMBODIMENTS

In order to assist the reader, an outline of the description and a summary of abbreviations is provided immediately below.

### I. Introduction

A. Glossary of Terms, Abbreviations, and Definitions
1. Table of Selected Terms and Abbreviations
2. Definitions

B. General Discussion of Invention

C. Underlying Bases for Invention

D. Overview of Some Aspects of Improved Assay Normalization

### II. Discussion of Conventional Assumptions and Practices

A. Validity of Representation and Frequency Assumptions R, Fₘₒₗₑ, and Fₘₐₛₛ

B. Validity of Prior Art Belief that for a Particular Gene mRNA Transcript Comparison Assay, (NASR)=(ACR)=(T-DGER)

C. Validity of Prior Art Belief that (ACR)=(T-DGER) for a Particular Gene Comparison
Validity of the relationship (N-DGER)=(ACR)=(T-DGER) when the first tacit assumption is invalid
Retrospective normalization of prior art measured particular gene N-DGER for SCR. An example
Validity of relationship (N-DGER)=(ACR)=(T-DGER) when the second tacit assumption is invalid
Validity of relationship (N-DGER)=(ACR)=(T-DGER) when the third tacit assumption is invalid
Validity of relationship (N-DGER)=(ACR)=(T-DGER) when two or more tacit assumptions are invalid
Interpretation of prior art measured N-DGER values when the Assay SCR [symbol] ₁
Effect of the validity of the prior art belief and practice that essentially all mRNA transcripts in a eukaryotic cell possess significant poly A tracts, on the relationship (N-DGER)=(ACR)=(T-DGER)
Aggregate effect on the biological accuracy of a particular gene N-DGER value of SCR [symbol] ₁and PAF [symbol] [symbol] ₁assay values
Summary: Validity of relationship (N-DGER)=(ACR)=(T-DGER) for prior art Microarray and non-microarray gene expression comparison assays
Validity of prior art assumptions required for the accuracy of prior art clone counting method particular gene mF and mFR values
Application of the validity discussions for gene expression analysis assays of all kinds

D. Validity of Prior Art Belief that (NASR)=(ACR) for a Particular Gene Comparison
Does the assay NASR equal the ACR?
Characteristics of gene expression analysis assay compared LPN molecules
Assay factors which affect the relationship (NASR)=(ACR)
TSAR and PSAR of LPNs
CDP and effective CDP complexity
The MLD and MLDR assay factors
The assay factor PL-HKR
The assay factor PS-HKR
The assay factor PSAR
The assay factor LLSR
The assay factors LD, LDR and PSSR
The association of signal generation complexes with hybridization immobilized indirectly labeled LPNs. The assay factors SBNR and SSAR
Effect of TSAR, PSAR and LLSR on (NASR=ACR)
The effect of the label density ratio (LDR) on the relationship (NASR)=(ACR)
Effect of MLDR on the relationship (NASR)=(ACR) for a Microarray gene comparison of type 1 LPNs.

Effect of MLDR on the relationship (NASR)=(ACR) for a Microarray gene comparison of type 2 LPN
Effect of assay hybridization kinetic factors on the relationship (NASR)=(ACR) for Microarray type 1 and type 2 LPN comparisons
Effect of PCR amplification efficiency (E) or AE AE values on the relationship (NASR)=(ACR) for an RT-PCR
Is the prior art belief that (NASR)=(ACR) valid?
Interpretation of prior art produced NASR and N-DGER values when (NASR)=(ACR)
Overall effect of MLDR, PL-HKR, PS-HKR, PSAR, PSSR, LLSR, SBNR, and SSAR UNFs on the relationship (NASR)=(N-DGER)=(ACR)

E. Effect of all UNFs on the Validity of Prior Art Produced N-DGER Values when it is not Assumed that (ACR=T-DGER) or that (Acr)=(NASR)=N-DGER

F. Effect of UNFP Assay Values on the Interpretation of Prior Art Microarray Data Analysis and Data Mining Analysis and Systems Biology Analysis Results.

G. Validity of Assumptions Required for Prior Art Normalization Methods Used to Produce Prior Art Microarray and Non-Microarray Results
(i) Most genes which are active in both compared cell samples are unregulated
(ii) In the Microarray cell sample comparison there is a balance between Up and Down regulated genes
(iii) Assay results associated with unregulated particular genes can be identified and used to generate one or more normalization factors (NF) which will correctly normalize all other assay particular gene results
(iv) The genes spotted on the array represent a significantly large random selection of the total number of genes in the compared cell sample
(v) and (vi) The total RNA per cell and/or the total mRNA per cell is the same for each compared cell sample
(vii) One of more particular genes which are active in both compared cell samples are known to be unregulated (ie, the housekeeping genes), and the assay RASR results for such genes can be used to normalize the other gene comparisons in the assay to produce biologically correct assay NASR values
Summary. Validity of prior art normalization assumptions

H. Validity of Prior Art Interpretation of Microarray and Non-Microarray Assay Measured Particular Gene Expression Negative Results
Occurrence of false negative gene activity results and regulation direction miscalls associated with (ACR) [symbol] (T-DGER)
Do EA rule and (ACR) [symbol] (T-DGER) related false negatives occur in real life?
Interpretation of EA rule and (ACR) [symbol] (T-DGER) related false negative results
Deviations from the EA rule in prior art Microarray and non-microarray practice
Occurrence of false negative gene activity results and regulatory direction miscalls (RDMs) associated with (ACR) [symbol] (RASR)
Do (ACR) [symbol] (RASR) related false negative results occur in real life?
Interpretation of NF related false negative results associated with (ACR) [symbol] (RASR)
Interpretation of assay variable NF related false negative results associated with prior art gene expression activity comparison assays

I. Validity of Prior Art Normalization of Corroborative Non-Microarray Gene Expression Comparison Assay Results
Validity of prior art practice of validating Microarray results with non-microarray gene expression comparison analysis results

### III. Exemplary Description of Applications and Practices of the Present Invention

A.
Determination of absolute and relative number of cells in a sample
Determination of total RNA per cell and total mRNA per cell for a cell sample
Determination of SCR for a cell sample gene expression comparison assay. The direct comparison of sample cell RNAs
Determination of SCR for a cell sample gene expression comparison assay involving the direct comparison of cell sample RNA equivalents such as cDNA or cRNA
Determination of Microarray cDNA or cRNA CE values and SCR values
Simplification of determination of assay SCR value for Microarray and non-microarray assays. The artificial housekeeping gene (AHG) approach
Key basic requirements and assumptions for gene expression analysis and gene expression comparison RT-PCR assays
Determination of RT-PCR assay CE values for oligo dT primed or random primed cell sample cDNA preps
Determination of RT-PCR assay SCR values for compared cell sample oligo dT and random primed cell sample cDNA preps
Determination of the number of particular gene ACEs and SCR for an SG primed RT-PCR assay
Interpretation of measured cell sample SCR values
Interpretation of prior art RT-PCR measured particular gene RN, mRNA abundance, and N-DGER values
Examples of prior art assay determination of particular gene RN, mRNA abundance, and N-DGER values
Determination of PAFR value
Determination of cDNA synthesis yield fraction (YF), and cDNA synthesis efficiency (SE), for a cell sample cDNA prep
Determination of nucleotide lengths of the analyzed and/or compared RNA transcript LPN preps
Determination of nucleotide sequence and/or nucleotide composition for particular gene RNA transcripts or particular gene RNA transcript LPNs
Determination of the total nucleotide complexity (TNC) for a particular gene RNA transcript LPN
Determination of the total polynucleotide number (TPN) for the analyzed or compared particular gene RNA transcript LPN
Determination of total signal activity (TSA) for the analyzed or compared cell sample RNA transcript LPN prep
Determination of PSAR and LLSR assay values for directly labeled LPNs
Determination of average label density (ALD) for a cell sample LPN prep and the label density (LD) for a particular gene LPN
Determination of compared particular gene LPN hybridization kinetic differences
Determination of ECDP
Determination of MLD and MLD
Determination of LLNR
LLSR determination and normalization for direct label type 2 LPN comparisons
LLSR determination and normalization for indirect labeled type 2 L-LPN comparisons
SBNR determination and normalization
SSAR determination and normalization
Normalization of particular gene comparison assay measured results for unconsidered assay variable associated UNFs
Normalization of particular gene expression comparison assay results for prior art considered assay variables (CNFs)
Normalization of particular gene comparison assay results for CNFs and UNFs
Normalization of SAGE and other clone counting method measured particular gene expression assay results for differences in cell sample RNA contents: measuring normalizing for the cell sample total mRNA number (STM)
The use of the artificial housekeeping gene (AHG) approach for simplifying and improving the determination of and normalization for, pertinent UNFs and CNFs for SAGE and other clone counting methods
Application of discussions on NF determination and normalization and the use of the AHG approach to Microarray and non-microarray or clone counting SGDS, DGDS, and DGSS gene expression analysis of different RNA types

B. Production of Improved Gene Expression Comparison Analysis Results for Microarray, Non-Microarray, and Clone Counting Method SGDS, DGDS, and DGSS Comparisons of Viral Prokaryotic, Eukaryotic and Standard RNA Transcripts of all kinds

C. Practice of the Invention for SGDS mRNA Transcript or mRNA Transcript cDNA or cRNA Equivalent Comparison Assays
Improvement of prior art normalization process for direct label LPN assays by assay design and measurement
Improvement of the prior art normalization process for indirect label L-LPN assays by assay design and measurement of UNF and CNF assay values
Improvement of non-microarray northern blot, DOT blot and nuclease protection assay normalization process
Improvement of RT-PCR assay normalization process
Improvement of all gene expression comparison assay normalization processes and particular gene expression results by using both the A SCR and R SCR assay values for normalization
Improvement of SAGE measured cell sample analysis and cell sample comparison analysis normalization process and assay results by assay design and measurement
Producing Microarray and non-microarray, and clone counting method improved normalization processes and improved assay results for DGDS and DGSS mRNA transcript comparison assays, and SGDS, DGDS, and DGSS RNA transcript of any kind comparison assays
Invention improved gene expression analysis results and gene expression analysis comparison results “Improvement Ripple Effect”: Further practices of the invention
Computer implementation of methods for determining and using improved assay normalization techniques
Conclusion

### IV. References

### V. Comments on Contents of Disclosure

### I. INTRODUCTION

A. Glossary of Terms, Abbreviations, and Definitions

1. Table of Selected Terms and Abbreviations

| | |
|---|---|
| Abundance | The number of RNA transcripts per cell for a particular gene. Equivalent to |
| | the RNA copies per cell, or RNA CPC. |
| ACR | The assay concentration ratio (ACR) equals the ratio in the microarray or non- |
| | microarray assay hybridization solution or the RT-PCR assay PCR |
| | amplification step of, (the molar concentration of a particular gene's RNA |
| | transcripts or equivalents from a cell sample) ÷ (the molar concentration of the |
| | compared particular gene's RNA transcripts from the compared cell sample). |
| | Note that the ACR can refer to an SGDS, DGDS, or DGSS comparison. |
| AE | Amplicon equivalent. A particular gene DNA or RNA molecule which can be |
| | used to produce the particular gene DNA amplicon molecule of interest by |
| | PCR amplification. An AE molecule can be designated an mRNA AE, an |
| | RNA of any kind AE, a cDNA AE, or a cRNA AE. |
| AE·AE | Amplicon equivalent PCR amplification efficiency. A particular gene or |
| AE·AER | standard AE·AE value is equal to (the number of particular gene or standard |
| | amplicon molecules produced in the assay in a known number of amplification |
| | cycles) ÷ (the number of particular gene or standard amplicons which would |
| | be produced in the same number of cycles when the PCR amplification |
| | efficiency (E) is one). In short, (AE·AE) = (1 + particular gene or standard |
| | assay E value)^{N} ÷ (2)^{N}, where N is the number of PCR amplification cycles. |
| | For a particular gene or standard comparison, the (AE·AER) = (AE·AE value |
| | for one particular gene or standard) ÷ (the AE·AE value for the compared |
| | particular gene or standard). |
| AE·CE or | A cell sample amplicon equivalent cell equivalent (ACE). For a particular |
| ACE | gene RNA or cDNA the ACE value is equal to the number of moles of the |
| | particular gene RNA transcript molecules which are present in an intact |
| | sample cell. The particular gene RNA ACE value equals the particular gene |
| | cDNA ACE value when the R and Fmole assumptions are valid. |
| AE·CN | The number of particular gene or standard RNA transcript AE cDNA |
| AE·CNR | molecules produced in the RT-PCR assay RT step from the RNA present in |
| | the RT step. AE·CNR is equal to the ratio of the compared particular gene or |
| | standard AE·CN values. |
| AE·RN | The number of particular gene or standard RNA transcript molecules present |
| AE·RNR | in an RT-PCR RT step. AE·RNR is equal to the ratio of the compared |
| | particular gene or standard AE·RN values. |
| AE·SE | The particular gene or standard AE cDNA synthesis efficiency (AE·SE). For |
| AE·SER | a particular gene or standard cDNA AE prep, (AE·SE) = (AE·CN ÷ AE·RN). |
| | The AE·SER for a particular gene or standard comparison is equal to the |
| | ratio of the compared particular gene or standard AE·SE values. |
| AHG RNA | A standard RNA or DNA which is used to produce an artificial housekeeping |
| or DNA | gene for a cell sample. |
| AHGR | Artificial housekeeping gene ratio. The AHGR is equal to, (the AHG |
| | abundance for one cell sample) ÷ (the AHG abundance for a compared cell |
| | sample). The AHGR equals the T-DGER for the AHG comparison, and is |
| | also equal to the SMAR for the AHG comparison. |
| ALD | Average label density for LPN. The ALD for a cell sample LPN prep is equal |
| ALDR | to the average number of direct or indirect label molecules per nucleotide |
| | base. ALDR is equal the ratio of the compared cell sample LPN ALD values. |
| AMPLICON | A particular gene or standard product DNA molecule produced by PCR |
| | amplification. |
| CAV | Prior art considered or visible assay variable. An assay variable which is |
| | known to the prior art and considered for the normalization of prior art gene |
| | expression analysis and gene expression comparison assay results. |
| CCN | cDNA cell equivalent number. The number of cell sample cDNA CEs |
| CCNR | produced in the RT step of the assay. CCNR is equal to the ratio of the |
| | compared cell sample CCN values. |
| cDNA YF | cDNA or cRNA synthesis yield fraction. cDNA YF is equal to the ratio for an |
| cRNA YF | RT reaction of, (the total amount of cDNA produced) ÷ (the amount of |
| | template RNA present). cRNA YF is equal to the ratio in the cRNA |
| | amplification solution of, (total cRNA produced) divided (by the amount of |
| | input template DNA). |
| CDP | The complementary detection polynucleotide. A CDP molecule is a spot |
| | immobilized polynucleotide molecule which is used to detect and quantitate |
| | the presence of particular gene LPN molecules in an assay hybridization |
| | solution. (See eCDP). |
| CE | A cell sample cell equivalent is the amount of cell sample nucleic acid or |
| | nucleic acid equivalent derived therefrom, which represents one sample cell or |
| | average sample cell. Such a nucleic acid CE may be an RNA or any kind CE, |
| | such as a T-RNA CE, a mRNA CE, or a particular gene RNA transcript of any |
| | kind CE. Such a nucleic acid equivalent CE may be a cDNA or cRNA CE |
| | derived from an RNA transcript of any kind, such as a T-RNA cDNA or |
| | cRNA CE, or a mRNA cDNA or cRNA CE, or a particular gene RNA |
| | transcript of any kind cDNA or cRNA CE. |
| C-HKR | Assay nucleic acid hybridization condition related hybridization kinetics ratio |
| | for a comparison of particular gene RNA, cDNA or cRNA LPNs. The C- |
| | HKR is a global CNF and affects all particular gene comparisons in the assay |
| | the same way. The C-HKR is a measure of the ratio of (the hybridization |
| | kinetics associated with all of the compared particular genes for one cell |
| | sample) ÷ (the hybridization kinetics associated with all of the compared |
| | particular genes in the compared cell compared particular genes in the |
| | compared cell sample). |
| CLR | The compared LPN nucleotide length ratio. The CLR is equal to the ratio of, |
| | (the nucleotide length of the synthesized particular gene RNA transcript |
| | cDNA LPN molecule) ÷ (the nucleotide length of the RNA template used to |
| | synthesize the cDNA LPN). |
| CNF | Prior art considered or visible assay variable associated normalization factor. |
| | A CNF is prior art known and it is often determined and normalized for. The |
| | CNFs include, but are not limited to, C-HKR, ARR, spatial, print tip, print |
| | plate, intensity, scale, AE·AE. (See NF) |
| CNFP | CNF assay values product. For an assay, the CNFP is equal to the product of |
| | the assay values for all of the assay pertinent CNFs associated with the assay. |
| CPC | RNA transcript copies per cell. For a particular gene RNA transcript in a cell, |
| | the CPC equals the abundance value. |
| DGE | Differential gene expression generally refers to the concept that the same |
| DGER | particular gene can be expressed to a different extent in different cells. In |
| N-DGER | addition, different particular genes in different cells (DGDS), and different |
| T-DGER | particular genes in the same cell (DGSS), can also be differentially expressed. |
| | Such a difference in gene expression between compared particular genes is |
| | generally described in terms of a DGE ratio or DGER. A DGER value which |
| | has been normalized for one or more assay variables is termed a N-DGER. |
| | The biologically accurate DGER value for a cell sample comparison is termed |
| | the true DGER or T-DGER. |
| DGDS | Different genes different cell sample (DGDS), and different gene same cell |
| DGSS | sample (DGSS). DGDS designates the comparison of the expression extents |
| | of different particular genes from different cell samples. DGSS designates the |
| | comparison of the expression extents of different particular genes in the same |
| | cell sample. (See also SGDS) |
| Direction | A change in a particular genes expression extent can result in a higher |
| of Gene | abundance or a lower abundance for the particular gene RNA transcript in a |
| Regulation | cell. A gene is upregulated when its RNA transcript abundance increases, and |
| | downregulated when the abundance decreases, and unregulated when the |
| | abundance is unchanged. |
| E | Efficiency of amplification value for a particular amplicon in a PCR |
| | amplification reaction. |
| EA Rule | Equal addition of RNA rule. Prior art gene expression comparison assays |
| | almost always compare equal amounts of cells sample RNA or mRNA. |
| ECDP | Effective CDP. The nucleotide length of a CDP molecule which is |
| | complementary to and can hybridize with, the particular gene LPN molecules |
| | in the assay hybridization solution which the CDP is designed to detect. |
| Equivalent | cDNA or cRNA which is derived from cell sample RNA, and represents the |
| cDNA | cell sample RNA in the assay. Also cDNA or cRNA which is derived from a |
| or cRNA | particular gene RNA transcript, and represents the particular gene RNA |
| | transcript in the assay. |
| False | Refers to a situation where a particular gene RNA transcript is present in a cell |
| Negative | sample RNA prep, but its presence is not detected by the assay. |
| Result | |
| Fmole | Mole frequency. Refers to the mole frequency of a particular gene RNA |
| | transcript or the cDNA or cRNA equivalents derived therefrom, in cells or in a |
| | cell sample RNA preparation derived from the cells, or in a cDNA or cRNA |
| | equivalent preparation derived from the cell RNA. |
| Global | An assay variable which affects all particular gene comparison results in the |
| Assay | assay to the same quantitative extent. (See non-global assay variable) |
| Variable | |
| Global NF | A normalization factor (NF) which is associated only with global assay |
| | variables. For an assay, there is only one assay value for each different global |
| | NF. A global NF assay value affects all particular gene comparison results in |
| | an assay in the same quantitative way. (See non-global assay variable) |
| HCN | High cell sample number. For a microarray or non-microarray cell sample |
| | comparison, the compared cell sample which is represented by the most cells. |
| | When the EA Rule is used for the assay, this cell sample has the lowest total |
| | RNA content per cell, or lowest total mRNA content per cell. |
| Intensity | A prior art known and normalized for non-global NF. |
| CNF | |
| JDA, JDAR | Just detectable abundance level for a cell sample in an assay. For a gene |
| | expression analysis assay the JDA is the lowest RNA transcript abundance |
| | level for a cell sample which can be detected by the assay. For a gene |
| | expression comparison assay, the JDAR is the ratio of the compared JDA |
| | values. |
| JDQ, JDQR | Just Detectable Quantity of a particular gene mRNA or cDNA or cRNA in a |
| | gene expression assay for a cell sample. JDQ can be measured in terms of the |
| | concentration of particular gene nucleic acid which is just detectable above |
| | background in an assay. The JDQR for a cell sample comparison is equal to |
| | the ratio of (the JDQ value for one compared cell sample) divided by (the |
| | JDQ value for the other compared cell sample). |
| LCN | Low cell sample number. For a microarray or non-microarray cell sample |
| | comparison, the compared cell sample which is represented by the least cells. |
| | When the EA Rule is used for the assay, this cell sample has the highest total |
| | RNA content per cell or the highest total mRNA content per cell. |
| LD | Label density. The LD for a particular gene RNA LPN molecule or cDNA or |
| LDR | cRNA equivalent LPN molecule, is equal to the number of label molecules per |
| | LPN nucleotide, which is associated with the particular gene LPN molecules. |
| | For a particular gene LPN comparison, the LDR is the ratio of the compared |
| | particular gene LD values. The LD is a non-global assay variable, which is |
| | associated with the non-global UNF PSSR, and also can affect the non-global |
| | UNFs PSAR and PS-HKR. |
| LLN | LPN label number. The LLN is associated with Type 2 LPNs, and is equal to |
| LLNR | the number of direct or indirect label molecules which are associated with |
| | each cell sample LPN molecule. For a cell sample comparison the LLNR is |
| | equal to the ratio of the compared cell sample LLN values. The LLN is a |
| | global assay variable. |
| LLS | Label signal activity per LPN molecule. The LLS is associated only with |
| LLSR | Type 2 LPNs, and is equal to the label signal activity which is associated with |
| | each LPN molecule in the cell sample LPN prep. For a Type 2 LPN the LLS |
| | value for each particular gene LPN in a cell sample LPN prep is the same. For |
| | a cell sample LPN comparison the LLSR is equal to the ratio of the compared |
| | cell sample LPN LLS values. The LLS value for each compared cell sample |
| | LPN can be the same or different. The LLSR is a global UNF. |
| LPN | Labeled polynucleotide. An LPN molecule is an RNA, DNA, cDNA, or |
| | cRNA molecule which is associated with direct or indirect label molecules. |
| L-LPN | A ligand labeled LPN molecule. An indirectly labeled LPN molecule. |
| mF | The mRNA transcript frequency. A measure of the frequency of occurrence |
| mFR | of a particular mRNA transcript in a population of mRNA transcripts of all |
| | kinds which is present in a cell or cell sample. The mF for a particular gene |
| | mRNA transcript in a cell is equal to the ratio of, (the number of particular |
| | gene mRNA transcript molecules per cell) ÷ (the total number of mRNA |
| | transcripts of all kinds in the cell). In short, for a particular gene mRNA |
| | transcripts, (mF) = (abundance) ÷ (STM). For a particular gene comparison, |
| | the mFR is equal to the ratio of the compared cell sample particular gene mF |
| | values. The mF varies for different particular gene mRNA transcripts in a |
| | cell. |
| mTN | The mRNA Transcript Number. Herein, MTN is used interchangeably with |
| RN. | mRNA Transcript Number. MTN used interchangeably with RN. |
| MLD | Maximum LPN nucleotide length detectable. The MLD for a particular gene |
| MLDR | RNA transcript LPN, is equal to the maximum nucleotide length of the |
| | particular gene LPN molecule(s) which can associate with one CDP molecule |
| | as a result of hybridization. For a particular gene LPN comparison, the |
| | MLDR is equal to the ratio of the compared MLD values. The MLD is |
| | associated with non-global assay variables, and the MLDR is a non-global |
| | UNF. |
| mTN | The mRNA transcript number. Herein mTN is used interchangeably with RN. |
| NAS | Normalized assay signal for a particular gene RNA transcript expression assay |
| NASR | result. The NAS for a particular gene RNA transcript expression analysis in |
| | an assay is derived by normalizing the assay measured raw assay spot signal |
| | activity (RAS) associated with the particular gene RNA transcript expression |
| | analysis, for pertinent assay variables, and/or assay variable associated NFs. |
| | For a particular gene RNA transcript comparison, the NASR value is equal to |
| | the ratio of the compared particular gene NAS values. A particular gene assay |
| | measured and normalized NASR value will equal the particular gene T-DGER |
| | value when the NASR value is validly and completely normalized for all |
| | pertinent assay variables. Prior art produced particular gene NASR values are |
| | believed to be biologically accurate, and therefore equal to the particular gene |
| | T-DGER value. |
| NF | Normalization factor. An NF is associated with non-global and/or global |
| | assay variables, and can be prior art known and considered, that is a CNF, or |
| | prior art unconsidered, that is a UNF. Each particular gene RNA transcript |
| | comparison assay result must be normalized for all pertinent NFs which are |
| | associated with the particular gene comparison. The NFs which are described |
| | herein include, the CNFs, C-HKR, spatial, print tip, print plate, intensity, |
| | scale, and the UNFs SCR, PAFR, MLDR, PL-HKR, PS-HKR, PSAR, PSSR, |
| | LLSR, SBNR, SSAR, and STMR. |
| NFP | NF assay values product. For an assay the NFP is equal to the product of the |
| | assay values for all assay pertinent CNFs and UNFs. In short, (NFP) = (CNFP) |
| | (UNFP). |
| Non-Global | A NF which is associated with one or more non-global assay variables. For a |
| NF | particular non-global NF there may be different assay values for the NF which |
| | are associated with different particular gene comparisons in the same assay. |
| | An assay value for an NF may be associated with only a subset of the |
| | particular gene comparisons in an assay. (See global NF) |
| NS·cRNA | Non-specific cRNA. Cell sample cRNA preps often contain a significant |
| | amount of cRNA which is not specific for the cell sample cDNA template the |
| | cRNA was produced from. |
| One Label | Each cell sample LPN prep is labeled with the same ligand or signal |
| Assay | generating molecule. For cell sample comparisons two separate microarrays |
| | must be used, and two separate hybridization reactions must be done. |
| PA mRNA | Polyadenylated mRNA. mRNA which is associated with a significantly long |
| | PA tract exists only in eukaryotic cells as PA mRNA. It is generally believed |
| | that virtually all eukaryotic cell mRNA molecules are associated with a |
| | significant 3′ PA tract. |
| PAF | Polyadenylated particular gene mRNA fraction. The PAF for a particular gene |
| PAFR | mRNA transcript in a cell is equal to the fraction of the particular gene mRNA |
| | transcript in the cell which is significantly polyadenylated. For a cell sample |
| | particular gene comparison the PAFR is equal to the compared particular gene |
| | PAF values. The PAFR is a non-global UNF. |
| Pertinent NF | For a particular gene RNA transcript comparison in an assay, a pertinent NF is |
| | one which is associated with assay variables which will cause the particular |
| | gene comparison assay result to deviate from assay or biological accuracy, |
| | when the assay value for the NF deviates significantly from one. |
| PG | Abbreviation for particular gene. |
| PGC | Abbreviation for particular gene RNA transcript or equivalents comparison. |
| PL-HKR | LPN nucleotide length difference related hybridization kinetics ratio for a |
| UNF | particular gene RNA transcript or cDNA or cRNA equivalent LPN |
| | comparison. The PL-HKR is a non-global UNF. |
| Print Tip | Replicate microarray CDP spots printed on the microarray by different print |
| CNF | tips can give different assay results, which are normalized for by the print tip |
| | non-global CNF. |
| Print Plate | Microarray CDP spots from a particular microtiter plate well are subpar and |
| CNF | must be normalized for. The print plate CNF is a non-global CNF. |
| PSA | The PSA represents the label signal activity associated with a particular gene |
| PSAR | RNA transcript or cDNA or cRNA equivalent LPN which is present in a cell |
| | sample LPN prep. The PSA value for a particular gene LPN is measured in |
| | terms of the signal activity per microgram of LPN. For a particular gene |
| | comparison the PSAR is equal to the ratio of the compared particular gene |
| | LPNs. The PSAR is a non-global UNF. |
| PS-HKR | Polynucleotide sequence difference related hybridization kinetics ratio for a |
| | particular gene RNA transcript or cDNA or cRNA equivalent LPN |
| | comparison. The PS-HKR is a non-global UNF. |
| PSS | Particular sequence duplex stability effect. For a particular gene RNA |
| PSSR | transcript or cDNA or cRNA equivalent LPN the PSS is expressed in terms of |
| | the fraction of the particular gene LPN which is associated with label density |
| | (LD) effects and which cannot form a stable hybridized duplex with the |
| | particular gene CDP, relative to the fraction of the same particular gene LPN |
| | which is not associated with LD effects and which can form a stable |
| | hybridized duplex with the particular gene CDP. The PSSR is equal to the |
| | ratio of the compared PSS values. The PSSR value is a non-global UNF. |
| R | Refers to the representation of particular gene RNA transcripts in an intact cell |
| | sample, relative to the representation in an isolated cell sample RNA prep, or a |
| | cell sample RNA LPN prep or a cell sample cDNA or cRNA equivalent LPN |
| | prep derived from the cell sample RNA prep. Prior art assumes that for assay |
| | compared cell sample RNA transcript LPNs, or cell sample cDNA or cRNA |
| | equivalent LPNs derived from the cell sample RNA, the R for each particular |
| | gene RNA transcript or cDNA or cRNA equivalent LPN in the cell sample |
| | LPN prep, is the same as in the intact cell sample. |
| RAS | The measured raw assay signal for a particular gene RNA transcript LPN or |
| RASR | cDNA or cRNA equivalent comparison. The RAS value for a particular gene |
| | LPN analysis is derived by subtracting the assay background associated with |
| | the particular gene spot from the total spot signal (TSS). The RASR is equal |
| | to the ratio of compared particular gene RAS values. |
| RCN | RNA sample cell equivalent (CE) number. The RCN is equal to the number |
| RCNR | of sample CEs which are present in the RT step of an assay. The RCNR is |
| | equal to the ratio of compared sample RCN values. The RCNR is also equal |
| | to the number of cell sample RNA CEs which are compared in an assay not |
| | associated with an RT step. |
| RDM | Regulation direction miscall. An RDM is associated with a particular gene |
| | RNA transcript comparison NASR or N-DGER assay result, when the |
| | direction of regulation change implicit in the ratio value is erroneous. |
| RIE | Sample cell RNA isolation efficiency. The RIE is equal to the fraction of the |
| | total |
| RIER | RNA, which is present in the intact sample cells processed, which is recovered |
| | as isolated RNA. For a cell sample comparison, the RIER is equal to the ratio |
| | of the compared cell sample's RIE values. |
| RN | The RNA transcript number. The RN for a particular gene RNA transcript |
| or | which is associated with the amount of cell sample or standard RNA which is |
| AE·RN | in the assay RT step, is equal to the number of particular gene RNA transcript |
| | molecules which is present in the assay RT step. |
| S | Standard RNA or DNA for microarray or non-microarray or clone counting |
| | method assays. |
| SAGE | Serial analysis of gene expression. The most widely used clone counting |
| | method. |
| SB | The signal generation complex (SGC) binding to ligand associated with a |
| | ligand labeled LPN which is immobilized on a surface. |
| SBN | Signal generation complex (SGC) binding number. The SBN is equal to the |
| SBNR | number of SGC molecules, which can stably bind to a single hybridization |
| | immobilized particular gene indirect label LPN molecule. The SBNR is equal |
| | to the ratio of compared particular gene SBN values. The SBNR is a non- |
| | global UNF. |
| Scale | A non-global CNF which adjusts the distribution width of the assay results. |
| SC | Sample cell number. The SC is equal to the number of a cell sample's RNA |
| SCR | or cDNA or cRNA cell equivalents (CE) which are analyzed in the assay |
| A-SCR | hybridization solution or PCR amplification step. For a cell sample |
| R-SCR | comparison, the SCR is equal to the ratio of the compared cell sample SC |
| | values. The SCR is a global UNF. The SCR and A-SCR are equivalent terms. |
| | The R-SCR reflects the sample cell number measured in terms of the haploid |
| | DNA content for the cell sample. |
| SE | cDNA or cRNA synthesis efficiency. The cDNA SE is equal to, (the number |
| SER | of cell sample cDNA CEs produced in the assay RT step) ÷ (the number of |
| | cell sample RNA template CEs present in the assay RT step). For a cell |
| | sample comparison, the cDNA SER is equal to the ratio of the compared cell |
| | samples cDNA SE values. The cRNA SE is equal to, (the number of cell |
| | sample cRNA CEs produced in the cRNA synthesis step) ÷ (the number of |
| | cell sample double strand cDNA template CEs present in the cRNA synthesis |
| | step). For a cell sample comparison, the cRNA SER is equal to the ratio of the |
| | compared cell sample cRNA SE values. When the R and Fmole assumptions |
| | are valid, the SER is associated with global assay variables. |
| SGC | Signal generation complex. SGC molecules are associated with indirect LPN |
| | assays. An SGC complex contains one or more signal generation molecules, |
| | and one or more molecules which specifically and strongly bind to a ligand |
| | molecule associated with the hybridization immobilized LPN molecule. |
| SGDS | Same gene different cell sample. SGDS comparisons compare the RNA |
| | transcript expression extents for the same particular gene, which is present in |
| | different cell samples. |
| SM | Standard RNA moles. The mole amount of a standard RNA which is added to |
| SMR | a cell sample RNA aliquot. For a cell sample comparison, the SMR is equal to |
| | the ratio of compared cell sample SM values. |
| SMA | Standard RNA abundance. The number of added standard RNA molecules per |
| SMAR | cell equivalent for a cell sample RNA aliquot. The SMA is equal to, (the SM |
| | for a cell sample aliquot) ÷ (the RCN for the same cell sample RNA aliquot). |
| | The SMAR for a cell sample RNA aliquot comparison is equal to the ratio of |
| | the compared cell sample RNA aliquot SMA values. For the cell sample |
| | comparison, the SMAR is equal to the AHGR and the T-DGER for the |
| | standard comparison. |
| Spatial CNF | A non-global CNF. The spatial CNF is often associated with surface |
| | heterogeneity related differences in assay signals. |
| SSA | SGC molecule signal activity. The SSA is equal to the quantitative amount of |
| SSAR | signal activity associated with an SGC molecule, which is immobilized in a |
| | particular gene spot, and is associated with an immobilized LPN from one cell |
| | sample LPN prep. The SSAR is equal to the ratio of compared particular gene |
| | SSA values. The SSAR is a non-global UNF, but can behave as a global |
| | UNF. |
| STM | Sample total mRNA. The STM is equal to the total number of mRNA |
| | molecules of |
| STMR | all kinds, which is present in a sample cell. The STMR is equal to the ratio of |
| | compared cell sample STM values. The STMR is a global UNF. |
| T-DGER | True differential gene expression ratio. T-DGER designates the actual DGER, |
| | which exists for the compared particular gene RNA transcripts in the |
| | compared cell sample or cell samples. |
| TNC | Total nucleotide complexity. The TNC for a particular gene RNA, or cDNA |
| TNCR | or cRNA equivalent LPN, represents the nucleotide complexity of the |
| | particular gene LPN molecule population which is present in a cell sample |
| | LPN prep. For any RNA transcript, the maximum possible TNC is equal to |
| | the nucleotide complexity of the said RNA transcript's undegraded RNA |
| | molecule. The TNCR is equal to the ratio of compared particular gene TNC |
| | values. The TNCR is associated with non-global assay variables. |
| TPN | Total LPN molecule number. The TPN for a particular gene RNA, or cDNA |
| TPNR | or cRNA equivalent LPN molecule population which is present in a cell |
| | sample LPN prep, represents the number or average number of individual |
| | particular gene LPN molecules in the particular gene LPN molecule |
| | population which are required to equal the TNC associated with the particular |
| | gene LPN molecule population. For a particular gene LPN which is the same |
| | nucleotide length as the undegraded particular gene RNA transcript, the TPN |
| | is equal to one. The TPNR for a particular gene LPN comparison is equal to |
| | the ratio of the compared TPN values. The TPNR can behave as a global or |
| | non-global assay variable. |
| TSA | Total signal activity. TSA is measured in terms of the total amount of signal |
| TSAR | activity per microgram of cell sample LPN molecules as measured under the |
| | assay signal measurement conditions. The TSAR for a sample comparison is |
| | equal to the ratio of the compared sample LPN prep's TSA values. Prior art |
| | regards the TSA as a global assay variable. |
| TSS | Total spot signal. The TSS is equal to the measured total spot raw assay signal |
| TSSR | obtained from a particular gene spot. The TSS for a particular gene spot is |
| | uncorrected in any way. The TSSR is equal to the ratio of compared particular |
| | gene TSS values. |
| Two Label | Each cell sample LPN prep is labeled with a different ligand or signal |
| Assay | generating molecule. For cell sample comparisons only one microarray and |
| | one hybridization reaction are required. |
| Type 2 LPN | A cell sample Type 2 LPN prep must have the following characteristics. The |
| | TPN must equal one for each particular gene RNA transcript, or cDNA or |
| | cRNA equivalent, LPN molecule population in the cell sample LPN prep. The |
| | LLN or LLS must be the same for each LPN molecule present in the cell |
| | sample LPN prep. |
| Type 1 LPN | A cell sample Type 1 LPN prep is any LPN prep which does not meet the |
| | requirement for a Type 2 LPN prep. |
| UCAV | Prior art unconsidered assay variable. The UCAVs are not considered by the |
| | prior art for normalization of prior art produced gene expression analysis and |
| | gene expression comparison assay results. |
| UNFP | UNF assay values product. For an assay the UNFP is equal to the product of |
| | the assay values for all of the pertinent UNFs which are associated with the |
| | assay. |

2. Definitions

As used herein in connection with nucleic acid preparations (e.g., RNA preparations purified from a cell sample) and samples, the term “characteristic data” refers to descriptive data concerning the nucleic acid molecules in the preparation or in a cell or cell sample, and in particular includes data describing amounts or molecule numbers for specified types of nucleic acid molecules in the nucleic acid molecule population in the preparation or cell or cell sample.

As used herein in reference to assays and assay kits and systems, the term “commercial” indicates that the kit, etc, is available for sale generally to individuals and/or business entities (e.g., profit and non-profit business entities). In contrast, the term “homebrew” indicates that the kit is not available for general sale. Typically such homebrew assays and materials are adapted for use by a particular laboratory and are not distributed beyond the particular business entity and/or collaborators.

As used in the context of the present invention, the terms “improved”, “improved results”, “improved assay” and like terms indicate that the reference item(s) or process has at least one better or more advantageous characteristic such that the item as a whole is better, more advantageous for a use, or otherwise preferred. Such improvement is commonly better in normalization, completeness of normalization, accuracy, reproducibility, interpretability, validity, and/or reliability and utility. Improvements in normalization are generally obtained according to the invention described herein by validly and/or more completely normalizing for pertinent UNFs and CNFs which were not previously completely and/or validly normalized for. Improvements in reliability may, for example, mean that the validity of the value, result, or process which was previously invalid or of uncertain validity have increased validity, e.g., either shown to be valid or correct, or the risk of invalidity or incorrect results or interpretations has been reduced. For example, the probability that a particular normalization factor or process is invalid may be reduced, even if not eliminated.

In the context of preparations of nucleic acid molecules, the term “improved nucleic acids” or “improved oligonucleotides” and like terms means that the molecules in the preparation are, on average, closer to a desired set of defined characteristics, e.g., defined length, sequence, composition, and absence of other damage. Generally for oligonucleotides the term indicates that the average density of damage in the nucleic acid molecules in the preparation is lower than under a comparison condition, e.g., differently synthesized preparations.

In the present context when used to refer to assay results, the phrase “known to be improved” means that the process of obtaining the results is based on normalization procedures which are known or shown to be valid or at least to be more likely to be valid than results produced using prior normalization procedures. Such procedures are distinguished, for example, from normalization procedures which are not known or shown to be valid (e.g., because they are based on assumptions which are themselves of unknown validity) or which are known or shown to be invalid (e.g., because they are based on assumptions which are known or shown to be invalid).

Improved validity, invalid, and uncertain validity, CNFs are defined in terms of the likelihood for a particular assay that the usual normalization assumptions which are necessary for the production of valid CNFs by prior art normalization methods which rely on these assumptions, are valid normalization assumptions for the assay. All or virtually all prior art microarray assay and high throughput gene expression analysis assay results are normalized by prior art normalization methods which rely on the validity of one or more of the usual necessary normalization assumptions.

Thus, one type of “improved CNF” is one where at least the likelihood of validity is increased for a CNF produced by a prior art normalization process which relies on the said normalization assumptions. Thus, for example, for a CNF of uncertain validity, if it is shown to be likely, even if not certain, that the usual necessary normalization assumptions are valid for an assay, it is therefore likely, even if not certain, that a prior art normalization method which relies on those assumptions will produce improved CNF s for the assay. Similarly, for a previously invalid CNF, if valid normalization assumptions are established, the CNF can then become an improved CNF. Another type of improved CNF is a CNF which is validly determined by a normalization method which does not rely on the prior art necessary normalization assumptions, e.g, a preferred method of doing this is to utilize multiple replicate artificial housekeeping genes (AHG) to facilitate valid CNF value determination.

An “invalid CNF” is one where it is likely but not necessarily certain that the usual normalization assumptions are invalid for the assay and therefore it is unlikely but not necessarily certain that a prior art method which relies on those assumptions will produce improved CNFs for the assay. Such designation of invalidity may, in some cases, be overcome by using alternative information to that which was initially used to characterize the CNF as an invalid CNF.

An “uncertain validity CNF” or “CNF of uncertain validity” is one where the likelihood of the validity of the usual normalization assumptions for the assay is uncertain and therefore it is uncertain whether a prior art method which relies on said assumptions will produce improved CNFs for the assay. In some cases, it may be possible with additional and/or different information to establish the validity or invalidity of the usual or alternative normalization assumptions.

Unless clearly indicated to the contrary (e.g., clearly limited to natural or unmodified molecules), the terms “nucleic acids” and “nucleic acid molecules” refer to molecules which are made of covalently linked chains of nucleotides and/or nucleotide analogs, and thus includes unmodified nucleic acid molecules, modified nucleic acid molecules, and analogs of nucleic acid molecules. The terms further include oligonucleotides as well as longer such chains, including without limitation, siRNAs, miRNS, and full-length mRNAs, cDNAs, and cRNAs.

Similarly, unless clearly indicated to the contrary, the term “oligonucleotide” is used to refer to relatively short nucleic acid molecules, that is molecules up to 200 linked nucleotides and/or nucleotide analogs. Such oligonucleotides may also be referred to as oligos or oligomers. Longer nucleic acid molecules may be referred to as polynucleotides, or simply nucleic acids or nucleic acid molecules.

The phrase, “obtain an NF value” or “determine an NF value” and like terms mean to measure the NF value (or other specified value or information) directly or to acquire it by some other means or from some other source, e.g., from a database or other reference source.

The term “pertinent” in the context of CNFs and UNFs designates a CNF or UNF which is associated with the assay and whose assay value must be obtained or directly or indirectly known in order to know whether it is necessary to normalize the assay result for the NF. When a pertinent NF assay value significantly deviates from one, then the gene expression assay result must be normalized for the pertinent NF.

The phrase, “prior art normalization process which relies on the usual necessary prior art assumptions for validity” and phrases of like import refers to a normalization process commonly utilized by the prior art which relies on the validity of one or more necessary assumptions for its validity. These prior art necessary assumptions are extensively discussed in the body of this disclosure in the section entitled “VALIDITY OF ASSUMPTIONS REQUIRED FOR PRIOR ART NORMALIZATION METHODS USED TO PRODUCE PRIOR ART MICROARRAY AND NON-MICROARRAY RESULTS”.

A “valid prior art CNF normalization process” and a “validated prior art CNF normalization process” are normalization processes for which the usual assumptions necessary for the valid determination of one or more assay pertinent CNF values are, respectively, known to be valid, and likely to be valid or known to be valid.

Conversely, an “invalid prior art CNF normalization process” refers to a prior art CNF normalization process for which one or more of the usual assumptions necessary for the direct or indirect valid determination of one or more assay pertinent CNF values, are invalid.

Reference to “a prior art normalization method determined CNF value which is known to be valid” refers to pertinent assay CNF value which has been or may be directly or indirectly determined by a prior art normalization process which is known to be a valid normalization process.

In the context of this invention, a “directly determined NF value” for a particular NF is an NF value which represents the quantitative assay value associated with one particular NF. An “indirectly determined NF value” for a particular NF, is one where the quantitative value for the NF is not determined directly, but is part of a determined quantitative assay value which represents the combined effect of two or more different pertinent NFs.

In the context of comparisons between values (e.g., total mRNA content per cell, or total number of mRNA transcripts per cell), unless otherwise specified, the term “significantly” indicates that the values differ to a statistically significant extent which is also substantial in the context of the particular assay. Further, specifically in the context of differences in total mRNA content per cell, or total number of mRNA transcripts per cell, indication that such difference is “not primarily due” to a specified cause or condition means that the specified cause or condition is responsible for less than ½ of the magnitude of the difference. In this same context, the phrase “expressed only in the compared sample which is associated with the larger measured value” means that the particular gene(s) are not expressed or not detectably expressed in cells from one of the two compared samples and are substantially and meaningfully expressed in cells of the other compared sample. Thus, it does not necessarily mean that there was absolutely no expression in the one set of cells, it only means that the expression in one set was insignificant compared to the expression level in the other.

B. General Discussion of Invention

The invention relates to all or nearly all prior art microarray and non-microarray and clone counting gene expression and gene expression comparison methods, and the assay results obtained with these methods. These include, but are not limited to, nucleic acid based microarray and macroarray methods, dot blot, northern blot, nuclease protection, various forms of reverse transcriptase PCR (RT-PCR), various forms of differential display, and various forms of clone counting methods. The invention relates in part to the incorporation of some mode of practice of the invention into such gene expression and gene expression comparison methods practiced by the prior art.

The invention further relates to all, or nearly all, applications, which utilize one form or the other of the assay results from gene expression and gene expression comparison methods of all kinds. Such assay results include, but are not limited to, gene expression results, gene expression comparison results, gene expression profile results, gene expression data mining results, and systems biology results. Said applications include, but are not limited to, all biological organisms such as eukaryotes, prokaryotes, viruses, and therefore microbes, plants, and animals of all kinds. The invention relates broadly to biological research and development of virtually all kinds, and to medical, agricultural, environmental, industrial, and manufacturing, applied, and service, applications, which are related to biology.

More specifically the invention relates to virtually all areas of biological research and development which include but are not limited to, physiology, genetics and gene regulation, epidemiology, evolution, ecology, endocrinology, immunology, nutrition, toxicology, oncology and cancers of all kinds, stem cell studies related to embryogenesis and differentiation, organ and tissue and cell in vitro studies of all kinds, organ and tissue and cell transplantation of all kinds, virology, microbiology, pathogenesis of all kinds, diseases of all kinds, and products and services which are associated with biological research and development.

The invention further relates to a large number of agricultural related applications. These include, but are not limited to, the following. Essentially all areas of basic, applied, and industrial agricultural research and development, including the just described biological research and development areas. The areas of developing naturally and genetically improved plants and animals and bacteria for food production and other purposes. The areas of plant and animal diseases of all kinds, and disease mechanisms, and host-pathogen interactions. The areas of the discovery, development, validation, production, and use, of plant and animal antiviral agents, antimicrobial agents, antifungal agents, pesticides, plant and animal growth agents, and agricultural pharmaceutical agents of all kinds. The areas of agricultural ecology and toxicology. Products and services which are associated with the above-described areas of application.

The invention further relates to a large number of medical, both human and veterinary, related applications. These include, but are not limited to, the following. Essentially all areas of basic, applied, and industrial, medical research and development, including the above-described biological research and development areas. The pathogenesis, prevention, diagnosis, treatment, and cure of: infectious and non-infectious diseases of all kinds; genetic and non-genetic diseases of all kinds; nutritional diseases of all kinds; central nervous system diseases of all kinds, including psychiatric conditions; cancers and tumors of all kinds; cardiac diseases of all kinds; other tissue or organ diseases of all kinds; immunologic diseases of all kinds; toxic compound related diseases of all kinds; fetal or differentiation related diseases of all kinds; addictive diseases of all kinds; other diseases of all kinds. Diagnostic tests for the above-described diseases. Products and services, which are associated with research and development associated with a disease or with the diagnosis, prevention, control, treatment, or cure, of a disease.

More specifically, the invention relates to most steps in the overall process of human and veterinary drug development, which includes the development of antimicrobial, and antiviral agents as well as other drugs. Such steps include, but are not limited to, the following. The discovery and identification of drug candidates. The evaluation of the specificity, toxicity, and efficacy, of drug candidates. The development of drug candidate related diagnostic tests. The improvement and/or optimization of drug candidate's specificity, and/or toxicity, and/or efficacy, and/or pharmacokinetic characteristics. The identification of clinical screening participants and the candidate drug's market niche. Quality control and quality assurance for drug production and manufacturing. The efficient prescription of drugs for patients and the evaluation of the effectiveness of drug treatment for the patient.

In addition the invention relates to the characterization, quality control, and use, of organisms and their organs and tissues and cells, including primary cells and stem cells, as well as in vitro cultured organs and tissues and cells including, primary cultured cells and stem cells, for different aspects of the drug development process. This includes the use of gene knockout and other organisms, and their organs and tissues and cells, as well as in vitro cultured organs and tissues and cells, including primary cells and stem cells, and also includes interfering RNA treated gene knockout and other organisms, and their organs and tissues and cells, as well as in vitro cultured organs and tissues and cells, including primary cells and stem cells, for use in the different aspects of the drug development and use process.

The invention also relates to industrial and applied applications, which are related to biology. These include but are not limited to, the following. Many of the above-described applications for biological, agricultural, medical, and drug development areas of application which relate to water quality, food quality, public health, ecology, including environmental and marine concerns, toxicology, forensics, diagnostics of many kinds, technology development, quality assurance and control. Also, standards for the development, production, or manufacture of applied products, and various services associated with the above areas of application.

In addition to the improvements in assay results described herein, the invention can also utilize the methods and compositions described in Kohne, U.S. Provisional Appl. 60/689,985, Kohne, U.S. patent application Ser. No. 11/38,203 and Kohne, U.S. patent application Ser. No. 11/383,198, each of which is hereby incorporated herein by reference in its entirety, including without limitation methods for providing improved oligonucleotide preparations and the resulting compositions, and methods for providing improved assay results including higher order application results.

C. Underlying Basis for Invention

The practice of the invention produces gene expression analysis assay results which, relative to prior art results, are by virtue of being known to be properly normalized, improved in one or more of the assay result characteristics, quantitation, accuracy, interpretability, reproducibility, intercomparability, likelihood of validity, utility, and biological correctness. The underlying bases for the said inventions improved gene expression analysis results, and the methods and means of the practice of the invention are rooted in: (a) The identification of, determination of the assay values for, and the consideration of during normalization for, certain biological and experimental global and non-global assay variables which are pertinent to microarray and non-microarray and clone counting gene expression analyzes for cell sample RNA transcripts of all kinds, and for such SGDS, DGDS, and DGSS gene expression RNA transcript expression analysis comparisons for RNA transcripts of all types, and which are not considered and taken into account by the prior art for the normalization of prior art microarray, non-microarray, and clone counting method, gene expression analysis assay results. (b) The biological and experimental assay factors which cause these prior art unconsidered global and non-global assay variables to occur. Herein, these prior art hidden or unconsidered microarray and non-microarray gene expression analysis assay variables are termed unconsidered assay variables, or UCAVs. Herein, the prior art visible assay variables which are taken into account for the normalization of prior art microarray and non-microarray gene expression analysis results, are termed considered assay variables, or CAVs. (c) Knowledge of the validity of the prior art assumptions which are required in order to produce prior art gene expression analysis and gene expression comparison results which are accurately normalized for prior art known and considered assay variable NFs.

The underlying bases for the invention's improved results and the practice of the invention method and means include, but are not limited to the following.
(i) Knowledge of the existence of the biological and experimental assay factors which cause the UCAVs to be associated with a gene expression analysis assay result.
(ii) Knowledge of whether a particular said biological or experimental assay factor causes global or non-global assay effects.
(iii) Knowledge of the effect of the said biological and experimental assay factors on the quantitation, accuracy, interpretation, intercomparability, reproducibility, utility, and biological correctness, of gene expression analysis assay results.
(iv) Knowledge of the effect of each said biological and experimental assay factor on the ability to produce gene expression analysis assay results which measure gene expression activity and gene expression differences in terms of the fundamental biological unit, the cell, or in terms of the DNA content of a cell.
(v) Knowledge of how to reduce or eliminate the effect of one or more of the said biological or experimental assay factors on gene expression analysis results.
(vi) Knowledge of how to obtain a quantitative measure for each of the said biological and experimental assay factors which are associated with a gene expression analysis assay.
(vii) Knowledge of how to express one or more of the said biological or experimental assay factors in terms of a defined and measured UCAV.
(viii) Knowledge of how to obtain a measure of the quantitative assay value for each UCAV associated with a gene expression analysis assay.
(ix) Knowledge of the effect of each separate said UCAV on the ability to produce gene expression analysis assay results which measure gene expression activity and gene expression differences, in terms of the fundamental unit, the cell.
(x) Knowledge of the effect of each UCAV on the quantitation, accuracy, interpretation, intercomparability, reproducibility, utility, and biological correctness of gene expression analysis results.
(xi) Knowledge of whether and when, each UCAV behaves as a global variable or non-global variable in a gene expression analysis assay.
(xii) Knowledge of how to determine a quantitative measure for a normalization factor (NF) value for a particular gene expression analysis assay result, for each UCAV or for combinations of different UCAVs.
(xiii) Knowledge of how to utilize each UNF or composite UNF to normalize particular gene expression assay results to produce improved gene expression analysis assay results.
(xiv) Knowledge of how to use the relevant UCAV normalization factors to produce improved normalized gene expression results, and difference in gene expression results, which are measured in terms of the fundamental biological unit, the cell.
(xv) Knowledge that data mining analysis and interpretation results of all kinds as well as systems biology analysis and interpretation results of many, if not all kinds, will be improved by the practice of the invention.
(xvi) Knowledge that the results from any process or application which utilizes gene expression analysis results, will be improved by utilizing improved gene expression analysis results.

D. Overview of Some Aspects of Improved Assay Normalization

As indicated above and described in greater detail below, the invention provides methods and means to obtain microarray and non-microarray and clone counting method gene expression and gene expression comparison assay results which are improved, relative to prior art microarray and non-microarray and clone counting method gene expression and gene expression comparison results. The practice of the invention provides microarray and non-microarray and clone counting method results which, as a result of being known to be improved in normalization relative to prior art microarray and non-microarray and clone counting method results, are improved with regard to quantitation and/or assay accuracy and/or biological accuracy and/or interpretability and/or intercomparability and/or utility, relative to prior art microarray and non-microarray and clone counting method gene expression analysis results. The practice of the invention is necessary in order to obtain gene expression analysis differential gene expression ratios for particular gene comparisons, which can be known to be biologically correct.

Because of the improved nature of such particular gene expression analysis results, the invention provides methods and means for obtaining improved global genome and genomic subset gene expression profiles for one or more sets of cell sample or tissue sample comparisons. The invention also provides methods and means for obtaining improved data mining (33) and systems biology (139) analysis results from the intercomparison, correlation, and analysis, of improved particular gene comparison assay results, and the improved genome profile results. Further, the invention provides methods and means for producing improved results from any process or application, which utilizes gene expression assay results, which can be improved by the practice of the invention.

The invention has application to all methods of gene comparison, and provides a variety of methods and means for obtaining improved microarray and non-microarray and clone counting method particular gene expression and SGDS, DGDS, and DGSS, particular gene RNA transcript of any kind expression comparison assay results. Such methods and means are broadly applicable to all kinds of cell sample or tissue sample gene expression comparisons or analyzes. Such methods and means can be used to produce improved particular gene expression and gene comparison results for cell sample and tissue sample comparisons which include, but are not limited to, the following. (a) Normal cells or tissues of all kinds and ages. (b) Differentiated cells and tissues of all kinds and ages. (c) Cells and tissues of all kinds in different cell cycle, growth, or metabolic states of all kinds. (d) Cells and/or tissues and/or organisms of all kinds associated with pathogenic or non-pathogenic viruses, cells, or organisms, of all kinds. (e) Cells and/or tissues and/or organisms of all kinds which are associated with a non-genetic or genetic disease state of any kind. (f) Cells and/or tissues and/or organisms of all kinds associated with a genetic change of any kind, whether created by man or nature. (g) Cell and/or tissues and/or organisms associated with or treated with bioactive, drug, toxic, non-toxic, mutagenic, inhibitor, or nutrient compounds, of all kinds, or any other chemical compounds, or combinations of such compounds. (h) Cells and/or tissues and/or organisms of all kinds associated with non-chemical treatments of all kinds such as radiation, temperature, mechanical, and stresses of all kinds. (i) Cultured cells of all kinds associated with substances or conditions which can affect cell growth rates, cell cycle stage, the cell cycle distribution profile, cell size, cell recombinant and other protein production capability, cell adherence to surface, cell morphology, cell differentiation, and other cell characteristics, and such substances and conditions include but are not limited to, pCO₂, pO₂, pH, stir rates and shear forces, osmotic pressure, redox potential, carbohydrate levels, growth factors, steroids and other hormones, lipids and fatty acids, amino acid levels, eicosanoids and eicosanoids precursors, cations, anions, cytokines, vitamins, nucleic acid precursors, and others.

The invention's method and means for producing improved microarray and non-microarray particular gene expression and gene expression comparison results include, but are not limited to, the following.

(i) Method and means for producing gene expression analysis and gene expression analysis comparison results which are known to be improved relative to prior art gene expression analysis and gene expression comparison analysis results, and such improved results include, but are not limited to, RN and abundance values for RNAs of all types, DGER values for SGDS, DGDS, and DGSS particular gene RNA expression comparison analyzes for RNAs of all types, cell sample gene RNA expression profiles for RNAs of all types, gene expression analysis and gene expression comparison analysis, gene expression profile data mining and analysis results of all kinds, and systems biology analysis results of all kinds which involve gene expression comparison results.

(ii) Method and means for producing gene expression analysis results which are more completely normalized relative to prior art gene expression analysis and gene expression comparison analysis results, and are thereby known to be improved relative to prior art produced gene expression analysis and gene expression comparison analysis results.

(iii) Methods and means to obtain cell, or cell sample, gene expression, and differences in gene expression, results measured in terms of the fundamental biological unit, the cell.

(iv) Method and means to obtain cell, or cell sample, or tissue sample, gene expression and differences in gene expression results, measured in terms of the amount of DNA per haploid or diploid cell for the compared cells, or cell samples.

(v) Methods and means for identifying and determining biological and experimental gene expression analysis assay factors, which can be responsible for the occurrence of certain prior art unconsidered assay variables.

(vi) Methods and means for identifying prior art unconsidered assay variables (UCAV) associated with prior art gene expression analyzes assays, which must be normalized for in order to obtain biologically correct gene expression analysis results, which are known to be correct.

(vii) Methods and means for determining a measure of the quantitative value for each gene expression analysis assay relevant unconsidered assay variable (UCAV) normalization factor UNF, which is associated with the assay.

(viii) Methods and means for evaluating the validity of the assumptions required for the validity of the prior art normalization for the prior art considered assay variables, and the interpretation of the prior art normalized assay results.

(ix) Method and means for reducing the assumptions required in order to interpret normalized gene expression analysis assay results.

(x) Method and means for improved, more complete normalization of gene expression comparison assay results.

(xi) Methods and means for improving the design of gene expression analysis assays, in order to minimize or eliminate the effect of one or more prior art considered or unconsidered assay variables on the assay results.

(xii) Method and means for improving the design of gene expression analysis assays to more efficiently obtain improved assay results.

(xiii) Method and means for improving the validity of the process of corroborating gene expression analysis normalized results obtained with one gene expression analysis method, with normalized gene expression analysis results obtained with a different gene expression analysis method.

(xiv) Method and means for retrospectively evaluating the validity of the prior art gene expression analysis normalized results with regard to quantitation, accuracy, interpretability, intercomparability, utility, and completeness of normalization.

(xv) Method and means for identifying and making known that certain prior art normalized gene expression analysis results, believed by the prior art to be correct and completely normalized, are incorrect and incompletely normalized.

(xvi) Method and means for identifying and making known that certain prior art normalized gene expression analysis results, believed by the prior art to be correct and completely normalized, cannot be known to be correct and completely normalized or not, and are not interpretable.

(xvii) Method and means to evaluate and make known the validity of prior art gene expression analysis corroboration results with regard to quantitation, accuracy, interpretation, intercomparability, and utility, and completeness, of normalization.

(xviii) Method and means for retrospectively improving the normalization of certain prior art gene expression analysis normalized assay results, which have been made known to be incompletely normalized or invalidly normalized.

(xix) Method and means for reducing or eliminating UCAV related erroneous differential gene expression ratio results, and associated erroneous regulation direction results, obtained from gene expression comparison analysis assays.

(xx) Method and means for retrospectively reducing or eliminating UCAV related erroneous differential gene expression ratio results, and associated erroneous regulation direction results present in prior art gene expression comparison analysis results.

(xxi) Method and means for identifying the occurrence of prior art considered and unconsidered assay variable related false negative assay results, and associated regulation direction miscalls, in gene expression analysis assays.

(xxii) Method and means for reducing and/or eliminating the occurrence of prior art considered and unconsidered assay variable related false negative results and associated regulation direction miscalls, in gene expression analysis assays.

(xxiii) Method and means for retrospectively identifying the occurrence of prior art considered and unconsidered assay variable related false negative results and associated regulation direction miscalls, in prior art gene expression assays.

(xxiv) Method and means to incorporate one or more of the aspects of the practice of the invention into virtually all prior art gene expression analysis methods.

(xxv) Method and means to discover and identify one or more true unregulated genes which are generally present in cells and cell samples, and which can be used to obtain improved normalized gene expression results. That is, can be used as a general use housekeeping gene.

(xxvi) Method and means to identify one or more true unregulated genes which are present in particular cells and cell samples, and which can be used to obtain improved normalized gene expression analysis results. That is, can be used as a limited use housekeeping gene.

(xxvii) Method and means to identify one or more different genes, which have a constant extent of regulation in different particular cells or cell samples, and such genes can be used to obtain improved normalized gene expression analysis results. That is, can be used in essentially the same manner as a limited use housekeeping gene.

(xxviii) Method and means for the design and incorporation into a gene expression analysis assay, of known amounts of one or more exogenous control polynucleotide molecules per compared sample cell for each compared cell sample, and which can be used to obtain improved normalized gene expression analysis results which are measured in terms of the fundamental biological unit, the cell. In other words, methods and means for creating one or more artificial true unregulated or regulated housekeeping genes in each compared cell sample, or one or more artificial constant extent of expression genes in each compared cell sample of a gene expression analysis assay.

As pointed out above, the invention has application to virtually all methods of gene expression and gene expression comparison analysis, and provides methods and means to produce improved particular gene expression and gene expression comparison results, and improved more accurate and more complete global and genomic subset gene expression profiles, for cell and tissue sample analyzes and comparisons of any kind. Such cell and tissue sample analyzes and comparisons include those listed above in the discussion on the invention methods and means for obtaining improved particular gene expression analysis and gene expression comparison results.

The methods and means for producing improved gene expression profile results include, but are not limited to means and methods (i)-(xxviii) described above.

The invention also provides methods and means to produce improved results from the intercomparison and analysis of one or more improved gene expression analysis global genomic, or genomic subset, gene expression profiles. Such improved results are herein termed improved data mining results. The inventions methods and means for producing improved data mining analysis and improved systems biology analysis results include, but are not limited to, the above discussed means and methods (i) thru (xxviii), and the following.

(xxix) Method and means for improving gene expression analysis data mining analysis and interpretation results of all kinds and systems biology analysis and interpretation results of all kinds.

(xxx) Method and means for retrospectively evaluating the validity of prior art gene expression analysis data mining and systems biology results with regard to quantitation, accuracy, interpretability, intercomparability, utility, and biological correctness.

(xxxi) Method and means for the improved more complete and accurate identification of genes with similar gene expression activity within a cell sample or tissue sample, or across a set of cell samples or tissue samples, or across multiple sets of cell samples or tissue samples, as for example, those cell samples or tissue samples (a)-(i) described above.

(xxxii) Method and means for the improved identification of genes with different expression activity within a cell sample or tissue sample, or across a set of cell samples or tissue samples, or across multiple sets of cell samples or tissue samples, as for example, those cell samples or tissue samples (a)-(i) described above.

(xxxiii) Method and means for the improved identification of groups of genes with similar global genomic and/or genomic subset gene expression profiles across a set of cell samples or tissue samples, or across multiple sets of cell samples or tissue samples, as for example, those cell samples or tissue samples (a)-(i) described above.

(xxxiv) Method and means for the improved identification of co-regulated genes within a cell sample or tissue sample, or across a set of cell samples or tissue samples, or across multiple sets of cell samples or tissue samples, as for example, those cell samples or tissue samples (a)-(i) described above.

(xxxv) Method and means for the improved identification of common patterns of gene expression within a cell sample or tissue sample, or across a set of cell samples or tissue samples, or across multiple sets of cell samples or tissue samples, as for example, those cell samples or tissue samples (a)-(i) described above.

(xxxvi) Method and means for the improved identification of common regulatory networks within a cell sample or tissue sample, or across a set of cell samples or tissue samples, or across multiple sets of cell samples or tissue samples, as for example, those cell samples or tissue samples (a)-(i) described above.

(xxxvii) Method and means for incorporating one or more aspects of the practice of the invention into virtually all prior art gene expression analysis result data mining method analyzes and/or systems biology based analyzes.

The invention provides methods and means to produce improved particular gene expression analysis results, and provides methods and means to produce improved global genomic and genomic subset gene expression profiles from the improved particular gene expression analysis results. In addition the invention provides methods and means to produce improved data mining analysis results and improved systems biology analysis results from the improved particular gene expression analysis results, and the improved global genomic and genomic subset gene expression profiles. The invention further provides methods and means for improving the results of any application, which utilizes one or more of the improved gene expression analysis results or improved data mining and/or systems biology analysis results described above. Such applications are very broad and include, but are not limited to, the areas of application of the methods and means of the invention described in the Field of Invention section. The invention's methods and means for producing improved results for these areas of application include, but are not limited to, the above discussed means and methods (i)-(xxxvii), and the following. For the description of the following means and methods, the term improved gene expression analysis results, refers to one or more of the methods of the invention improved, particular gene expression analysis or gene expression comparison results, improved global genomic or genomic subset gene expression analysis profiles, or improved data mining results or improved systems biology analysis results.

(xxxviii) Method and means for improving the results of any application which utilizes or produces gene expression analysis results of any kind which can be improved by the practice of the invention.

(xxxix) Method and means for retrospectively evaluating the validity of prior art application results which produces or utilizes gene expression analysis results which can be improved by the practice of the invention, with regard to quantitation and/or accuracy and/or interpretability, and/or utility and/or biological correctness.

(xl) Method and means for improving the results of biological research and development applications of all kinds which produce or utilize gene expression analysis results which can be improved by the practice of the invention, with regard to quantitation and/or accuracy and/or interpretation and/or intercomparability and/or utility and/or biological correctness.

(xli) Methods and means for improving the results of agriculture related applications of all kinds which produce or utilize gene expression analysis results which can be improved by the practice of the invention, with regard to quantitation and/or accuracy and/or interpretability and/or intercomparability and/or utility and/or biological correctness.

(xlii) Methods and means for improving the results of human medical, and prokaryote, and eukaryote, and virus medical related applications of all kinds which utilize or produce gene expression analysis results which can be improved by the practice of the invention with regard to quantitation, and/or accuracy, and/or interpretation and/or intercomparability and/or utility and/or biological correctness.

(xliii) Method and means for improving the results of in vitro cultured cell related applications, including primary culture; stem cell culture, and continuous cell culture related applications of all kinds, which produce or utilize gene expression analysis results which can be improved by the practice of the invention, with regard to quantitation and/or accuracy and/or interpretation and/or intercomparability and/or utility and/or biological correctness.

(xliv) Method and means for improving the results of in vitro cultured tissue or organ culture applications which produce or utilize gene expression analysis results which can be improved by the practice of the invention, with regard to quantitation and/or accuracy and/or interpretation and/or intercomparability and/or utility and/or biological correctness.

(xlv) Method and means for improving the results of gene knockout organism and their organs and tissues and cells, including primary and stem cells as well as in vitro cultured organs and tissues and cells, including primary and stem cells, applications of all kinds including drug discovery and development, which produce or utilize gene expression analysis results which can be improved by the practice of the invention, with regard to quantitation and/or accuracy and/or interpretation and/or intercomparability and/or utility and/or biological correctness.

(xlvi) Method and means for improving the results of interfering RNA and/or other regulatory RNA or DNA treated knockout and other organisms and their organs and tissues and cells, including primary and stem cells, as well as interfering RNA and/or other regulatory RNA or DNA treated knockout and other in vitro cultured organs and tissues and cells, including primary and stem cells, applications of all kinds including drug discovery and development and validation and toxicology evaluations, which produce or utilize gene expression analysis results which can be improved by the practice of the invention, with regard to quantitation and/or accuracy and/or interpretation and/or intercomparability and/or utility and/or biological correctness.

(xlvii) Methods and means for improving the results of industrial and applied applications of all kinds, which produce or utilize gene expression analysis results which can be improved by the practice of the invention, with regard to quantitation and/or accuracy and/or interpretation and/or intercomparability and/or utility and/or biological correctness.

(xlviii) Methods and means for improving the results of any human, veterinary, or other drug development processes which produce or utilize gene expression analysis results which can be improved by the practice of the invention, with regard to quantitation and/or accuracy and/or interpretation and/or intercomparability and/or utility and/or biological correctness.

(xlix) Methods and means for improving the results of any drug candidate discovery and identification process which produces or utilizes gene expression analysis results which can be improved by the practice of the invention, with regard to quantitation and/or accuracy and/or interpretation and/or intercomparability and/or utility and/or biological correctness.

(l) Methods and means for improving the results of any process for the evaluation of a drug candidates specificity and/or toxicity and/or efficacy and/or pharmokinetic characteristics, which produces or utilizes gene expression analysis results which can be improved by the practice of the invention, with regard to quantitation and/or accuracy and/or interpretation and/or intercomparability and/or utility and/or biological correctness.

(li) Methods and means for improving the results of any process for the evaluation and/or improvement and/or optimization of drug candidates specificity and/or toxicity and/or efficacy and/or pharmokinetic characteristics, which utilizes or produces gene expression analysis results which can be improved by the practice of the invention, with regard to quantitation and/or accuracy and/or interpretation and/or intercomparability and/or utility and/or biological correctness.

(lii) Methods and means for improving the results of any process for the identification of suitable clinical screening participants for the clinical evaluation of a candidate drug, which utilizes or produces gene expression analysis results which can be improved by the practice of the invention, with regard to quantitation and/or accuracy and/or interpretation and/or intercomparability and/or utility and/or biological correctness.

(liii) Methods and means for improving the results of any process for the identification of a candidate drugs market niche, which utilizes or produces gene expression analysis results which can be improved by the practice of the invention, with regard to quantitation and/or accuracy and/or interpretation and/or intercomparability and/or utility, and/or biological correctness.

(liv) Methods and means for improving the results of any process for the quality control and quality assurance for candidate drug discovery or drug manufacturing, which produces or utilizes expression analysis results which can be improved by the practice of the invention, with regard to quantitation and/or accuracy and/or interpretation and/or intercomparability and/or utility and/or biological correctness.

(lv) Methods and means for improving the results of any process for drug prescription and and/or evaluation of the effectiveness of the drug for the patient use, which utilizes or produces gene expression analysis results which can be improved by the practice of the invention, with regard to quantitation and/or accuracy and/or interpretation and/or intercomparability and/or utility and/or biological correctness.

(lvi) Methods and means for improving the results of any drug discovery, drug identification, drug toxicity, drug specificity, drug efficacy, drug pharmokinetic, or other, diagnostic process or test which utilizes or produces gene expression analysis results which can be improved by the practice of the invention, with regard to quantitation and/or accuracy and/or interpretation and/or intercomparability and/or utility and/or biological correctness.

(lvii) Methods and means for incorporating the practice of the invention into all applications and processes which produce or utilize gene expression analysis results which can be improved by the practice of the invention.

(lviii) Method and means for incorporating the practice of the invention into all software programs for normalization and analysis of gene expression results, and for data mining and systems biology analysis, which utilize gene expression analysis results which can be improved by the practice of the invention, as well as the resulting software and related databases and data sets.

### II. DISCUSSION OF CONVENTIONAL ASSUMPTIONS AND PRACTICES

Following is a description and discussion of each UCAV and how the UCAV relates to prior art microarray and non-microarray gene expression analysis results. This discussion and description of UCAVs is done in the context of the validity of prior art microarray and non-microarray and clone counting method gene expression analysis practices and assay results. These discussions include the following.
(i) A description of each UCAV and the biological and experimental factors which cause each UCAV.
(ii) A discussion of the effect of each UCAV on the quantitation and/or accuracy and/or interpretation and/or reproducibility and/or intercomparability and/or utility and/or biological correctness of microarray and non-microarray gene expression analysis results.
(iii) The validity of prior art microarray and non-microarray gene expression analysis practices and assumptions on the quantitation and/or accuracy and/or interpretability and/or intercomparability and/or reproducibility and/or utility and/or biological correctness, of microarray and non-microarray gene expression analysis results.

This discussion will start with the validity of the prior art assumptions on representation and frequency.

A. Validity of Representation and Frequency Assumptions R, Fmole, and Fmass

Virtually all prior art microarray and non-microarray gene expression analyzes routinely practice and believe the validity of the following assumptions. The representation and frequency of occurrence of each particular gene mRNA present in the intact cell or cell sample, is essentially identical to the representation and frequency of occurrence of each particular gene mRNA present in the total RNA isolated from the cell or cell sample, and in the total mRNA isolated from the cell or cell sample total RNA. In other words, it is assumed that isolation of the cell or cell sample total RNA and mRNA does not result in a significant change in the representation or frequency of occurrence of particular gene mRNAs, relative to the intact cell or cell sample. Further, it is assumed that the process of producing cell or cell sample mRNA LPN preparations from cell or cell sample total RNA or total mRNA, does not result in a significant change in the representation or frequency of occurrence of particular gene mRNAs, relative to the intact cell or cell sample. Prior art practices and believes that these assumptions must be valid in order to obtain certain gene expression analysis results, which are biologically correct. The validity of these representation (R) and frequency (F) assumptions is discussed below in terms of mRNA transcripts. However, the discussion also applies directly to different RNA transcripts of all types and to microarray and non-microarray SGDS, DGDS, and DGSS, assays of all types.

The basic representation and frequency assumptions were discussed earlier in the Background section. For simplicity, the term representation will be referred to as R, while the term frequency will be referred to as F. The terms mRNA Fmole and mRNA Fmass were defined earlier, and those definitions will be used in this discussion. In addition, the total RNA isolated from a cell or cell sample is herein referred to as T-RNA, and the PA mRNA fraction isolated from undegraded T-RNA is referred to as isolated mRNA or I•mRNA, while the PA mRNA fraction isolated from degraded T-RNA is referred to as degraded isolated mRNA or DI•mRNA.

The validity of the basic R and F assumptions requires that for a particular gene mRNA, the (R in the intact cell sample)=(R in the T-RNA isolated from the cell sample)=(R in the I•mRNA isolated from the T-RNA). For isolated cell and cell sample T-RNA preps, the assumption is generally assumed to be valid for both undegraded and degraded T-RNA preps. While there is no hard evidence to prove that the R assumption is always valid, there is sparse evidence which suggests that the R assumption is at least largely valid with regard to isolated T-RNA, for most, if not all, particular gene mRNAs in degraded and undegraded sample T-RNAs. With the exception of a small number of particular gene mRNAs, which do not possess polyadenylate tracts, prior art also generally assumes that the R assumption for undegraded I•mRNA preps is valid. Again, there is evidence, which suggests that the R assumption is largely valid with regard to undegraded I•mRNA preps for many, if not most, particular gene mRNAs in a cell sample.

Prior art acknowledges that for DI•mRNA isolated from degraded T-RNA, the R assumption is not valid for the entire nucleotide sequence of each particular gene mRNA present in the T-RNA. Isolated cell sample T-RNA is often degraded (140-142). Depending on the degree of degradation, some particular gene mRNA molecules in the T-RNA prep may be represented by multiple sub-mRNA molecules, which do not represent full sized mRNA molecules. If the degree of degradation is great enough, all short and long mRNA molecules in the T-RNA prep will be fragmented, and each individual total mRNA sequence will be represented by multiple sub-mRNA molecule fragments. In such a situation the entire mRNA sequence is present in the T-RNA, but in multiple pieces. Even when the T-RNA is extensively degraded the R of each particular gene mRNA is the same as if the T-RNA were undegraded. Therefore, even for extensively degraded T-RNA the assumption is valid with regard to R. Almost all undegraded T-RNA mRNA molecules have a poly A tract attached to the mRNA 3′ end. The I•mRNA isolation procedure relies on the ability to isolate the mRNA molecules which are physically attached to a poly A tract. During the PA mRNA isolation from degraded T-RNA, only the portion of each particular gene mRNA sequence which is attached to a poly A tract will be isolated and present in the DI•mRNA. Thus, for an extensively degraded T-RNA prep, only the mRNA molecule or mRNA piece which represents the 3′ end of each particular gene mRNA nucleotide sequence, is present in the DI•mRNA. The 5′ end pieces will be missing from the DI•mRNA prep for each particular gene mRNA. For the DI•mRNA prep the R assumption will be valid for each particular gene mRNA 3′ end nucleotide sequence piece, and invalid for each particular gene mRNA 5′ nucleotide sequence piece. In contrast, for the undegraded I•mRNA prep the R assumption is valid for the entire nucleotide sequence length of each particular gene mRNA.

The validity of the basic R and F assumption requires that for a particular gene mRNA in a cell sample T-RNA prep, the (Fmole in the cell sample)=(Fmole in the T-RNA isolated from the cell sample)=(Fmole in the I•mRNA isolated from the T-RNA prep), and that the (Fmass in the cell sample)=(Fmass in the T-RNA isolated from the cell sample)=(Fmass in the I•mRNA obtained from the T-RNA). In reality, these assumptions have not been proven to be valid or invalid. However, prior art gene expression analysis practitioners assume and practice that the mRNA Fmole and Fmass assumptions are valid for cell sample isolated T-RNA preps. This will also be assumed for this discussion, and generally assumed herein. As discussed earlier, prior art also believes and practices that virtually all of the mRNA molecules present in an undegraded eukaryotic T-RNA prep are PA mRNA's. This will also be assumed here.

In this context, for each particular gene mRNA present in an undegraded T-RNA prep, the (Fmole or Fmass in the T-RNA prep)=(the Fmole or Fmass in the I•mRNA prep isolated from the T-RNA), and the Fmole and Fmass assumptions are valid. However, as discussed, T-RNA is often degraded. Depending on the degree of degradation, some or all particular gene mRNA molecules in the T-RNA prep may be represented by multiple sub-mRNA molecules, which do not represent full sized mRNA molecules. If the degree of degradation is great enough, all short and long mRNA molecules in the T-RNA prep will be fragmented, and each individual total mRNA sequence will be represented by multiple sub-mRNA molecule fragments. In such a situation the entire mRNA sequence is present in the T-RNA, but in multiple pieces. Even when the T-RNA is extensively degraded the Fmole and Fmass of each particular gene mRNA is the same as if the T-RNA were undegraded. Therefore, even for extensively degraded T-RNA the F assumption is valid with regard to mRNA Fmole and Fmass. All undegraded T-RNA mRNA molecules have a poly A tract attached to the mRNA 3′ end. The I•mRNA isolation procedure relies on the ability to isolate the mRNA molecules which are physically attached to a poly A tract. During the PA mRNA isolation from degraded T-RNA, only the portion of each particular gene mRNA sequence which is attached to a poly A tract will be isolated and present in the DI•mRNA. Thus, for an extensively degraded T-RNA prep, only the mRNA molecule or mRNA piece which represents the 3′ end of each particular gene mRNA nucleotide sequence, is present in the DI•mRNA. The 5′ end pieces for each particular gene mRNA will be missing from the DI•mRNA prep. For the DI•mRNA prep then, the Fmole assumption will be valid for each particular gene mRNA 3′ end nucleotide sequence piece, and invalid for each particular gene mRNA 5′ end nucleotide sequence piece or pieces. For the same DI•mRNA prep, the Fmass assumption will be generally invalid for both the 3′ end pieces which are present and the 5′ end pieces which are missing.

Table 2 summarizes the validity of the assumptions for a particular gene mRNA which is present in degraded and undegraded T-RNA and isolated mRNA.

TABLE 2
Validity of Basic R and F Assumptions For Cell
and Cell Sample Isolated T-RNAs and mRNAs

| | | Validity For A Particular Gene mRNA in RNA Prep | | | | | |
|---|---|---|---|---|---|---|---|
| RNA | RNA | R | | Fmole | | Fmass | |
| Sample | Integrity | 3′ End | 5′ End | 3′ End | 5′ End | 3′ End | 5′ End |
| T-RNA | Undegraded | V | V | V | V | V | V |
| | And | | | | | | |
| | Degraded | | | | | | |
| I · mRNA | Undegraded | V | V | V | V | V | V |
| DI · mRNA | Degraded | V | NV | V | NV | NV | NV |

(i) V = Assumption is valid. NV = Assumption not valid.
(ii) The 3′ end and 5′ end, refers to whether the mRNA 3′ end and 5′ end are represented in the cDNA.

The validity of the basic F assumption requires that for each particular gene mRNA cDNA in a cell sample cDNA prep produced from T-RNA, the (R, Fmole and Fmass in the cDNA prep)=(R, Fmole and Fmass in the T-RNA prep). Whether these Basic R, Fmole, and Fmass assumptions are valid for cDNA preps produced from T-RNA preps, depends on whether the T-RNA prep is degraded, whether oligo dT primer or 3′ end specific gene primers or random primers are used to produce the cDNA prep from the T-RNA, and the nucleotide length of the oligo dT or 3′ end specific gene primed synthesized cDNA relative to the nucleotide length of the undegraded mRNA template molecules present in undegraded T-RNA. For this discussion, the situation for oligo dT primers will also represent the situation for 3′ end specific gene (SG) primers. Herein also, the ratio of (the nucleotide length of the synthesized cDNA molecule)÷(the nucleotide length of the mRNA template molecule used to produce the cDNA molecule), is termed the cDNA length ratio, or CLR. Note that when SG or oligo dT priming is used, a maximum of one cDNA molecule can be produced from each mRNA template molecule, but that not all mRNA template molecules may produce a cDNA molecule. Note further that when random priming is used, more than one different cDNA molecules are generally produced from each mRNA template, and essentially the entire mRNA template is represented in the cDNA.

Table 3 presents a summary of the effect of different combinations of the assay factors which can affect the validity of the basic assumptions for a particular gene mRNA cDNA which is present in a cell sample cDNA prep. The validity of the assumptions is determined with regard to whether the 3′ end and 5′ end of a particular gene mRNA is present in the cDNA prep. Since cell sample T-RNA and mRNA are often degraded, and oligo dT and random primers are often used, and the CLR value is often less than one for oligo dT primed cDNAs, each of the different combinations of assay factors presented in Table 3 has occurred often in prior art microarray and non-microarray gene expression comparison practice.

TABLE 3
Validity of R, Fmole, and Fmass, For Particular
Gene mRNA cDNA Molecules

| | | | | R and Fmole and Fmass For a Particular | | | | | |
|---|---|---|---|---|---|---|---|---|---|
| | | | | Gene mRNA cDNA in the cDNA Prep | | | | | |
| | | | | R | | Fmole | | Fmass | |
| RNA | RNA | Primer | | | 5′ | | 5′ | | 5′ |
| Sample | Integrity | Used | CLR | 3′ End | End | 3′ End | End | 3′ End | End |
| T-RNA | Undegraded | Oligo dT | 1 | V | V | V | V | V | V |
| | | Oligo dT | <1 | V | NV | V | NV | NV | NV |
| | Degraded | Oligo dT | 1 | V | NV | V | NV | NV | NV |
| | | | <1 | V | NV | V | NV | NV | NV |
| | Undegraded | Random | <1 | V | V | V | V | V | V |
| | Degraded | Random | <1 | V | V | V | V | V | V |
| Isolated | Undegraded | Oligo dT | 1 | V | V | V | V | V | V |
| mRNA | | Oligo dT | <1 | V | NV | V | NV | NV | NV |
| | Degraded | Oligo dT | 1 | V | NV | V | NV | NV | NV |
| | | | <1 | V | NV | V | NV | NV | NV |
| | Undegraded | Random | <1 | V | V | V | V | V | V |
| | Degraded | Random | <1 | V | NV | V | NV | NV | NV |

(i) V = Assumption is valid. NV = Assumption not valid.
(ii) The 3′ end and 5′ end refers to whether the cDNA represents the 3′ end and 5′ ends of the template mRNA.

The R and F assumptions are completely valid only under particular assay conditions. The majority of prior art cell sample cDNA preps are produced using oligo dT priming. In addition, prior art emphasizes the desirability of isolating and using undegraded T-RNA or mRNA for the production of such oligo dT primed cDNA. When oligo dT primer is used, the R and F assumptions can only be met when the T-RNA or I•mRNA are undegraded, and the cDNA synthesis CLR=1. However, it is known that for oligo dT priming of undegraded T-RNA, or I•mRNA, or an isolated particular gene mRNA transcript, the CLR value is virtually always significantly less than one (110). As a consequence, the R, Fmole, and Fmass assumptions are invalid for virtually all prior art produced cell sample cDNA preps which are oligo dT primed. Further, the Fmass assumption for such oligo dT primed cDNA preps is invalid for both the 3′ ends and 5′ ends of the mRNAs or cDNAs. In contrast, for such oligo dT primed cDNA preps the R and Fmole assumptions are likely to be valid for the 3′ end of the mRNAs or 5′ end of the cDNAs, for all poly A tract associated mRNAs.

The R, Fmole, and Fmass assumptions are essentially completely valid for cell sample cDNA preps produced with random primers from undegraded or degraded T-RNA, or undegraded isolated mRNA. This is shown in Table 2. The R, Fmole, and Fmass assumptions are invalid for cell sample cDNA preps, which are produced from DI•mRNA preps. In this situation, the Fmass assumption is invalid for both the 3′ ends and 5′ ends of the mRNA. Here, the R and Fmole assumptions are valid for the 3′ ends of the mRNAs, or 5′ ends of the cDNAs. Note that random primed cDNAs are somewhat underrepresented for the extreme 3′ end of a particular gene's mRNA.

Overall then, the R, Fmole, and Fmass assumptions are essentially always invalid for prior art cell sample cDNA preps produced by oligo dT priming, and are only valid for prior art cell sample cDNA preps produced by random priming of the T-RNA or undegraded isolated mRNA. However, the R and Fmole assumptions produced by random priming of T-RNA or undegraded I•mRNA are valid. However, for the oligo dT primed cell sample cDNA preps the R and Fmole assumptions are likely to be valid for the 3′ end mRNA nucleotide sequences near the priming site.

B. Validity of the Prior Art Belief that for a Particular Gene mRNA Transcript Comparison Assay, (NASR)=(ACR)=(T-DGER)

The validity of this prior art belief and practice requires that for a prior art particular gene mRNA transcript expression comparison assay, (the assay value for the particular gene mRNA transcript ACR value)=(the assay value for the particular gene mRNA transcript T-DGER value), and (the assay measured and normalized particular gene mRNA transcript N-DGER value)=(the assay value for the particular gene mRNA transcript ACR value). Since prior art gene expression analysis comparison assay practice involves almost exclusively the microarray, non-microarray, or cell counting method SGDS comparison of cell sample particular gene mRNA transcripts, this validity discussion will be in terms of the SGDS comparison of cell sample mRNA transcripts, unless otherwise noted. However, the discussion will be directly pertinent to SGDS, DGDS, and DGSS comparisons of cell sample RNA transcripts of all types.

C. Validity of Prior Art Belief that (Acr)=(T-DGER) for a Particular Gene Comparison

For this discussion on the validity of the relationship (ACR)=(T-DGER), it will be useful to assume that the relationship (NASR)=(ACR), is valid. Further, because by definition, (NASR=N-DGER) for an assay, and because prior art almost always reports gene expression comparison assay results in terms of the N-DGER, it will be useful to present this discussion in terms of the validity of the relationship (N-DGER)=(NASR)=(ACR)=(T-DGER), when it is assumed that (NASR)=(ACR). In other words, in terms of the validity of the relationship (N-DGER)=(T-DGER).

The validity of the relationship (N-DGER)=(T-DGER) for a particular gene mRNA transcript SGDS comparison is affected by the validity of each of the earlier discussed tacit assumptions one, two, and three. In order for the relationship (N-DGER)=(T-DGER) to be valid when it is assumed that (NASR)=(ACR), these three tacit assumptions must be valid, or the invalidity of each assumption must be compensated for by another assay variable value. In order to simplify this discussion it will be assumed that the prior art produced N-DGER value has been validly and accurately normalized for all pertinent assay variables except the tacit assumption being discussed. The validity of each of these assumptions, and the effect of the invalidity of each of these tacit assumptions on the validity of (N-DGER)=(T-DGER) for an assay, is discussed below, beginning with tacit assumption one. Each assumption will be discussed in the context of the almost universal prior art assay practice of the use of the EA Rule.

The Validity of the Relationship (N-DGER)=(ACR)=(T-DGER) when the First Tacit Assumption is Invalid.

The first tacit assumption specifies that for a gene expression comparison assay, each compared cell sample must have the same, or essentially the same, value for the amount of T-RNA or mRNA per cell. This assumption applies to prior art microarray and non-microarray assay SGDS and DGDS particular gene mRNA and all other cell sample RNA type transcript expression comparisons of all kinds, including those which directly compare cell RNAs, and those which are associated with the use of reverse transcriptase to produce T-RNA or mRNA equivalents such as cDNA or cRNA. Note that this first tacit assumption is not pertinent for microarray, non-microarray or clone counting DGSS gene RNA transcript expression comparisons of any kind.

As discussed in the Background section, significant naturally occurring differences in the amount of T-RNA and/or mRNA per cell are common for different cell samples of the same type, and different cell sample types. The magnitude of such differences depends on the cell's type, cell cycle stage, state of differentiation, growth conditions, and treatment conditions, as well as other factors. It is clear that prior art gene expression comparison assays of all kinds commonly compare cell samples, which have very significantly different values for the amount of T-RNA and/or mRNA per cell. Further, prior art gene expression comparison practice does not determine the T-RNA and/or mRNA content per cell for assay compared cell samples. In addition, little is known about the effect of various chemical and physical treatments on the amount of T-RNA and/or mRNA per cell values of the treated cells. Further, the amount of information available on the amounts of T-RNA per cell for different natural cells is relatively small, and there is even less information available concerning mRNA. As a result, the actual occurrence frequency for comparing cell samples with different or the same T-RNA and/or mRNA contents per cell cannot be known precisely, but is certainly high.

Prior art gene expression comparison assays of all kinds almost always employ the earlier discussed EA Rule to determine the amount of cell sample T-RNA or mRNA, or equivalents to compare in the assay. This rule specifies that equal amounts or masses of each cell sample's T-RNA, mRNA, or equivalents be compared in the assay. This then, is the assay context under which cell sample's which have different T-RNA and/or mRNA amounts per cell are compared.

It is clear that for many prior art gene expression comparison assays of all kinds, the first tacit assumption is invalid. Consequently, for such assays, the assay measured (N-DGER)≠(T-DGER). The effect of the invalidity of the first assumption on the assay N-DGER result is discussed and analyzed in detail below. It will be useful to present this discussion in terms of the prior art assay practice of using the EA Rule to determine the amount of cell sample RNA, or equivalents, to compare. Therefore, the discussion will focus on the effect of the use of the EA Rule on the relationship (N-DGER)=(T-DGER) when cell samples with different amounts of T-RNA and/or mRNA per cell are compared. For simplification, the discussion will concern the microarray assay comparison of cell sample isolated T-RNA preps, unless otherwise noted. However, the discussion will apply directly to microarray, non-microarray, and clone counting method assays of all kinds, as well as to SGDS and DGDS RNA transcript and RNA transcript equivalent of all kinds comparison assays.

In addition to the above, it will be assumed for this discussion that tacit assumptions two and three are valid. For this discussion then, the only assay variable is the use of the EA Rule in an assay situation where the first tacit assumption is invalid.

A consequence of the practice of the EA Rule for comparing cell samples which have different total RNA contents per cell, or total mRNA contents per cell, is that unequal numbers of each sample's cells are compared in the gene activity assay. In the assay the cell sample with the highest total RNA or mRNA content per cell will be the Low Cell Number (LCN) sample, while the cell sample with the lowest total RNA or mRNA content per cell will be the High Cell Number (HCN) sample. For a specific mRNA transcript present in each sample, this creates a situation where the relative amounts of each sample's mRNA transcripts which are present in the comparative assay, does not reflect the relative amounts of specific mRNA transcripts which are present in the average cell of each compared sample. Thus, relative to the actual situation present in the average cell of each compared sample, the amount of the LCN sample specific mRNA transcript present in the comparison assay is under-represented. A consequence of this is that in the resulting gene activity comparison assay, the specific mRNA transcripts from the HCN sample can be detectable, while those from the LCN sample can be undetectable, even though the numbers of specific mRNA transcripts per cell is equal to or higher than that in the HCN sample.

The effect of the practice of the EA Rule on the number of each samples cells which is compared in the assay can be illustrated using the earlier described comparison of rapid growing and slow growing bacterial cell samples. Herein, these will be termed RG and SG bacterial cell samples. Here, the total RNA content per cell of RG bacterial cells is ten times higher than that of SG bacterial cells. The EA Rule specifies that equal masses of total RNA from RG and SG cells must be compared. The number of SG cells in one specific mass amount of total RNA from SG cells is equal to, (the specific mass of SG cell total RNA compared)÷(x), where (x) is equal to the total RNA content per SG cell. Since the RG cells contain ten times more total RNA per cell than the SG cells, the amount of total RNA per RG cell is (10×). The number of RG cells in the same specific mass of total RNA from RG cells is then equal to, (the specific mass of RG cell total RNA compared)÷(10×). Thus, there are ten times more SG cells in the comparison than there are RG cells. Whenever the EA Rule is practiced for total RNA or total mRNA in a gene activity comparison of cell samples which have different total RNA contents per cell or total mRNA contents per cell, unequal numbers of cells will be compared. The practice of any rule which results in comparing a particular ratio of total RNA, total mRNA, or equivalents, from the cell samples, will also result in comparing unequal numbers of sample cells, except at the one unique ratio of sample RNA's which results in the comparison of equal sample cell numbers. For standard microarray and non-microarray methods where the EA Rule is almost always practiced; (a) the natural total RNA content per cell and total mRNA content per cell of the compared cell samples is often not the same; (b) the total RNA content per cell and total mRNA content per cell for the analyzed cell samples is unknown; (c) and therefore, the number of each cell sample's cells compared in the assay is almost always unknown. This situation makes it impossible to interpret certain prior art gene expression analysis results with regard to the biological accuracy of the particular gene N-DGER values. This is discussed below.

EA Rule related N-DGERs are widely believed to accurately reflect the actual differential gene expression ratios which are present in the cell samples being compared. For a particular gene, the T-DGER ratio which exists in two cell samples being compared, is equal to the ratio of, (the number of a gene's mRNA transcripts per cell for one sample)÷(the number of the same gene's mRNA transcripts per cell for the other sample). Standard microarray practice uses the EA Rule, and adds equal masses of each sample's total RNA to the hybridization solution, and by doing so, establishes a ratio in the hybridization solution of, (the number of one sample's gene mRNA transcripts which are present in the hybridization solution)÷(the number of the other sample's gene mRNA transcripts which are present in the hybridization solution). In a properly working microarray assay, this ratio is equal to the N-DGER value, which is experimentally obtained. This EA related, experimentally obtained N-DGER is currently regarded by the prior art as accurately reflecting the T-DGER of, (the number of gene mRNA transcripts per cell for one sample)÷(the number of gene mRNA transcripts per cell in the other sample). In other words, it is assumed that (the N-DGER)=(the T-DGER).

The problem with this interpretation of the N-DGER is embodied in the answers to two questions. First, does the EA Rule-related N-DGER always equal the T-DGER? Second, does the EA Rule-related N-DGER ever equal the T-DGER? The answers to the first and second questions are no, and yes, respectively. This is discussed below.

Significant differences in the total RNA content per cell, and mRNA content per cell, are common for different types of cells, depending on their type, cell cycle stage, state of differentiation or growth, and environment. By taking this into account, it is possible to demonstrate that the EA Rule related N-DGER values often do not accurately reflect the actual T-DGER values present in the cells compared. This can be illustrated by analyzing the microarray comparison of two cell samples, which have different, but known, total RNA contents per cell. One such system is a comparison of RG and SG bacteria, where it is known that the total RNA content per cell for RG bacteria is ten times higher than for SG bacteria (10, 11). Each of these bacteria populations is essentially a homogeneous population of cells of one type.

In the practice of the EA Rule, equal masses of total RNA from each bacteria sample are added to a microarray hybridization solution. The consequence of this is that the ratio in the hybridization solution of, (the number of RG cell equivalents)÷(the number of SG cell equivalents), is equal to 0.1. The number of sample RNA cell equivalents (CE) for one cell sample, is the number of sample cells, which contain the amount of total RNA added to the hybridization solution. The ratio in the microarray hybridization solution of, (the number of one sample's cell equivalents which are present)÷(the number of the other samples cell equivalents which are present), is termed the hybridization solution sample cell ratio, or SCR. In this illustration, the SCR is equal to 0.1. A microarray SCR of 0.1 means that an equal mass of SG bacteria cell total RNA represents ten times more bacteria cells, than an equal mass of RG cell total RNA. In this microarray cell comparison, the practice of the EA Rule dictates an (RG/SG) SCR equal to 0.1.

To further this illustration it will be assumed that in both RG and SG cells a particular gene is actively expressed, and that one copy of the gene's mRNA transcript is present in each RG and SG cell. For this gene, there is no difference in expression between RG and SG cells, and the T-DGER is equal to one.

In the practice of the EA Rule, equal masses of total RNA from each bacterial sample are added to a microarray hybridization solution. The consequence of this is that the resulting SCR is equal to 0.1, and this means that in the microarray hybridization solution there are ten times more SG cells than there are RG cells. Since both RG and SG cells contain one copy per cell of a particular gene's mRNA transcript, then in the hybridization solution the ratio of, (the number of the gene's mRNA transcripts present which originate from RG cells)÷(the number of the same gene's mRNA transcripts present which originate from SG cells) is equal to 0.1. In a properly working microarray assay, this ratio is equal to the N-DGER. This EA related, experimentally obtained N-DGER is in standard microarray practice, regarded as accurately reflecting the T-DGER present in the bacteria cell samples being compared. Further, the N-DGER value of 0.1 would be interpreted to mean that the particular gene was downregulated by ten fold in RG cells, relative to SG cells. In reality however, the gene was expressed at one copy per cell in both cell types. Clearly, in this situation the EA Rule practice results in a biologically erroneous N-DGER which is not equal to the T-DGER. Here the relationship between the N-DGER, the T-DGER, and the SCR, can be expressed as (T-DGER)=(N-DGER)÷(SCR). When (SCR=0.1), the N-DGER is ten times lower than the T-DGER for each gene which is active in both compared samples. In addition, the microarray miscalled the direction of gene expression change. Such a regulatory direction miscall is herein termed an RDM.

A similar analysis can be made by comparing purified total mRNA from growing and non-growing mouse fibroblast 3T3 tissue culture cell samples, which have different total mRNA contents per cell. The total mRNA content per cell of growing 3T3 cells is six times higher than that for non-growing 3T3 cells. Here when purified mRNA is compared, the value for SCR is 0.167, when SCR is defined in terms of (the number of growing cells present)÷(the number of non-growing cells present). Here, it is assumed that each growing cell contains six copies per cell of a particular genes mRNA transcripts, and the non-growing cells contain only one copy per cell of the same gene's mRNA transcript. In this instance, the practice of the EA Rule dictates that in a hybridization solution the ratio of, (the number of the gene's mRNA transcripts present which originate from growing cells)÷(the number of the same gene's mRNA transcripts present which originate from non-growing cells), is equal to one. The resulting N-DGER would then be equal to one, while the T-DGER is known to be equal to 0.167. This N-DGER of one would, in standard microarray practice, be regarded as accurately reflecting the T-DGER present in the 3T3 cell samples being compared. Further, the N-DGER would be interpreted to mean that the particular gene was expressed to the same extent in the two 3T3 cell samples, when in fact the gene was upregulated six fold in growing 3T3 cells. Here the relationship between T-DGER, N-DGER, and SCR can be expressed as (T-DGER)=(N-DGER)÷(SCR), and when SCR=0.167, then the N-DGER is six times lower than the T-DGER.

Because the above illustrations involved the comparison of two cell samples, each consisting of only one type of cell, the interpretation of the results is relatively straightforward. The following illustrations involve comparing natural heterogeneous populations of cells, that is, different mammalian tissue types. Each tissue is composed of multiple different cell types, and each cell type present consists of cells which may or may not be homogeneous with regard to growth stage, and stage of differentiation. In addition, the number or fraction of each different cell type present in the sample tissue is generally not known. However, for the purpose of the illustrations, each tissue will be treated as if it contained only one cell type. This is, in effect, the current microarray practice.

Table 1 indicates that the total RNA content per cell of the average rat adult liver cell is about 25 times greater than for a rat adult thymus cell. Here total RNA is compared and it is assumed that a particular gene is active in both tissues, and that there are ten copies of the gene's mRNA transcripts per liver cell, and one copy of the gene's mRNA transcript per thymus cell. Here, the EA Rule dictated SCR equals to 0.04 when the thymus cell number is present in the denominator. In this instance, the practice of the EA Rule dictates that in a hybridization solution the ratio of, (the number of the gene's mRNA transcripts present which originate from liver cells)÷(the number of the gene's mRNA transcripts present which originate from thymus cell), is equal to 0.4, and therefore the N-DGER will equal 0.4. Standard microarray practice would regard this (N-DGER=0.4) as correct, when in reality, the T-DGER=10. Further, the N-DGER would be interpreted as meaning that the liver gene was downregulated by 2.5 fold, when in fact; the liver gene is upregulated 10 fold. Here (T-DGER)=(N-DGER)÷(SCR).

This same method of analysis can be used to compare total RNA from cell populations, both of which have the same total RNA content per cell. In this instance, the EA Rule dictated SCR equals one. The date in Table 1 indicates that the total RNA content per cell is very similar for adult rat liver and pancreas tissue. For the purposes of this illustration, it will be assumed that both these tissues have identical total RNA per cell contents. Since both tissues are composed of multiple different cell types, the total RNA content per cell values will represent average values. Here it is assumed that each liver cell contains six copies per cell of a particular gene's mRNA transcript, while each pancreas cell contains only one copy per cell of the gene's mRNA transcript. Here, the practice of the EA Rule dictates that in a hybridization solution the ratio of, (the number of gene's mRNA transcripts present which originate from liver cells)÷(the number of the gene's mRNA transcripts present which originate from pancreas cells) is equal to six, while the T-DGER also equals six. Standard microarray practice would regard this N-DGER as being correct, and would interpret it to mean that the gene was upregulated six fold. In this situation where the EA Rule dictated SCR equals one, the practice of the EA Rule results in a correct N-DGER, which is equal to the T-DGER. Here, the relationship between N-DGER, T-DGER, and SCR, can be expressed as (T-DGER)=(N-DGER)÷(SCR). Since (SCR=1), then (T-DGER)=(N-DGER). Thus, in the practice of the EA Rule, whenever equal numbers of cells are compared, then (T-DGER)=(N-DGER), absent some other assay variable effect.

In the practice of the EA Rule, the SCR value is predictive of how far the N-DGER will deviate from the T-DGER. An SCR value of 0.1 or 10 for example, indicates that the N-DGER will deviate 10 fold from the T-DGER. If the total RNA content per cell of two samples is known, then the EA Rule related SCR is equal to the ratio of each samples total RNA content per cell. Note that this assumes that SCR is the only pertinent assay variable.

These examples demonstrate that when the (SCR≠1), then (T-DGER)≠(N-DGER), and when (SCR=1), then (T-DGER)=(N-DGER). This illustrates the problem with the interpretability of prior art produced the EA Rule related N-DGER values. The EA Rule related N-DGER may be obtained from a prior art microarray assay which has an (SCR=1), or it may not. Prior art microarray practice does not determine the SCR for a microarray cell comparison and the prior art gene expression analysis comparison of cell samples which have significantly different RNA per cell contents is very common. Consequently, there is no way of knowing when the (SCR=1), and when it doesn't, and therefore there is no way of knowing when these N-DGER are correct, and when they aren't. In this context, absent some knowledge of each EA Rule related microarray SCR value, both the quantitative extent and the direction of the prior art microarray gene expression measurements are uninterpretable.

An EA related microarray N-DGER for a gene does not always reflect the true direction of gene expression change or difference, that is, whether the gene is up, down, or not regulated. This was illustrated above in the bacteria, 3T3 cell, and tissue comparisons. Each of these examples involved just one assumed T-DGER for one gene. In order to better illustrate the effect of the practice of the EA Rule on the interpretation of the direction of gene expression change, a paper comparison of total RNA from RG and SG bacteria, and 3T3 cells, and total mRNA from growing and non-growing 3T3 cells, was done at many different T-DGERs. Each comparison then, involved the SCR dictated by the practice of the EA Rule, and multiple assumed T-DGERs. In the bacteria comparison, the total RNA content per cell of RG cells is ten fold higher than that of SG cells. For the 3T3 cell comparison, the total RNA content per cell of growing cells is four fold higher than that of non-growing cells, while the total mRNA content per cell is six times higher in growing cells. Tables 4, 5, and 6, present the results of this exercise. For the bacteria comparison, every N-DGER deviates ten fold from the correct T-DGER (Table 4). In addition, at certain T-DGERs values the EA Rule related N-DGER indicates that a gene is downregulated, when in reality the gene expression is upregulated. At another T-DGER value, the N-DGER will indicate no change in gene expression, when in reality the gene expression is upregulated 10 fold. At still another T-DGER, value the N-DGER indicates a 10-fold downregulation has occurred, when in reality no change in gene expression has occurred. Interestingly, while the quantitative value for each N-DGER always deviates 10 fold from its respective T-DGER, the N-DGER indications of upregulation are always 10 fold less than reality, and the N-DGER indications of downregulation are always 10 fold greater than reality. This occurs when the growing cell parameter is present in the numerator of the SCR, N-DGER, and T-DGER. The general pattern is the same for the 3T3 cell comparisons. In these cases the N-DGER differ less from reality because the SCR values are closer to one.

TABLE 4
Comparison of the Total RNA of RG and SG Bacteria

| | | ^{(b)}Experimental | Prior Art N-DGER | |
|---|---|---|---|---|
| ^{(a)}Assumed | Known | N-DGER | Based Assessment of | |
| T-DGER | SCR | Must Equal | Gene Activity | Reality |
| 100 | 0.1 | 10 | Upregulated 10 fold in | Upregulated 100 fold |
| | | | RG cells | in RG cells |
| 10 | 0.1 | 1 | No change | Upregulated 10 fold |
| | | | | in RG cells |
| 4 | 0.1 | 0.4 | Downregulated 2.5 fold | Upregulated 4 fold in |
| | | | in RG cells | RG cells |
| 2 | 0.1 | 0.2 | Downregulated 5 fold | Upregulated 2 fold in |
| | | | in RG cells | RG cells |
| 1 | 0.1 | 0.1 | Downregulated 10 fold | No change |
| | | | in RG cells | |
| 0.5 | 0.1 | 0.05 | Downregulated 20 fold | Downregulated 2 fold |
| | | | in RG cells | in RG cells |
| 0.1 | 0.1 | 0.01 | Downregulated 100 | Downregulated 10 |
| | | | fold in RG cells | fold in RG cells |
| 0.01 | 0.1 | 0.001 | Downregulated 1,000 | Downregulated 100 |
| | | | fold in RG cells | fold in RG cells |

^{(a)}All ratios represent (RG/SG)
^{(b)}(N-DGER) = (T-DGER) (SCR)

TABLE 5
Comparison of Growing and Non-Growing 3T3 Cells Total RNA

| | | | Prior Art | |
|---|---|---|---|---|
| | | Experimental | N-DGER Based | |
| Assumed | Known | N-DGER | Assessment of Gene | |
| T-DGER | SCR | Must Equal | Activity^{(a)} | Reality^{(a)} |
| 100 | 0.25 | 25 | G up 25x | G up 100x |
| 10 | 0.25 | 2.5 | G up 2.5x | G up 10x |
| 4 | 0.25 | 1 | No change | G up 4x |
| 2 | 0.25 | 0.5 | G down 2x | G up 2x |
| 1 | 0.25 | 0.25 | G down 4x | No change |
| 0.5 | 0.25 | 0.125 | G down 8x | G down 2x |
| 0.1 | 0.25 | 0.025 | G down 40x | G down 10x |
| 0.01 | 0.25 | 0.0025 | G down 400x | G down 100x |

^{(a)}G = Growing Cells Up = Upregulated Down = Downregulated x= Fold change in Gene Expression

TABLE 6
Comparison of Total mRNA From Growing and Non-Growing
Mouse Fibroblast 3T3 Cells

| | | | Prior Art | |
|---|---|---|---|---|
| | | Experimental | N-DGER Based | |
| Assumed | Known | N-DGER | Assessment of Gene | |
| T-DGER | SCR | Must Equal | Activity^{(a)} | Reality^{(a)} |
| 100 | 0.166 | 16.6 | G up 16.6x | G up 100x |
| 10 | 0.166 | 1.66 | G up 1.66x | G up 10x |
| 6 | 0.166 | 1 | No change | G up 6x |
| 5 | 0.166 | 0.83 | G down 1.2x | G up 5x |
| 4 | 0.166 | 0.66 | G down 1.5x | G up 2x |
| 2 | 0.166 | 0.33 | G down 3x | G up 2x |
| 1 | 0.166 | 0.166 | G down 6x | No change |
| 0.5 | 0.166 | 0.083 | G down 12x | G down 2x |
| 0.2 | 0.166 | 0.033 | G down 30x | G down 5x |
| 0.1 | 0.166 | 0.0166 | G down 60x | G down 10x |
| 0.01 | 0.166 | 0.00166 | G down 600x | G down 100x |

^{(a)}G = Growing Cells Up = Upregulated Down = Downregulated x= Fold change in Gene Expression

A comparison of Tables 5 and 6 indicates that the SCR for the EA Rule related 3T3 total mRNA comparison is significantly different from that of the 3T3 total RNA comparison. This disparity is due to the fact that the total mRNA in growing 3T3 cells increased by six fold while the total RNA increased only four fold. As a consequence, in this practice of the EA Rule a particular gene's N-DGER obtained from a total RNA comparison, will not equal the N-DGER for the same gene, which is obtained from a total mRNA comparison. This indicates that it cannot be assumed that the N-DGER obtained from comparing the total RNA from two cell samples will equal the N-DGER obtained from comparing the total mRNA from the same two cell samples. In this context, a situation may occur where the total RNA content per cell is identical in the samples compared, but the total mRNA content per cell in each sample is different. A comparison of these samples total RNA's with the practice of the EA Rule will result in an (SCR=1) and the experimentally obtained N-DGER will equal the T-DGER. In contrast, a comparison of these samples purified total mRNA's with the practice of the EA Rule will result in an (SCR≠1) and the experimental (N-DGER)≠(T-DGER).

Knowing the direction of a gene expression change is considered to be more important than knowing the absolute value of the DGE ratio (12). As discussed, the problems in interpretation of EA Rule related N-DGER concern both the magnitude and direction of gene expression extent changes which exist between samples. The practice of the EA Rule can produce N-DGERs which indicate that a gene is regulated in one direction, when in reality it is regulated in the other direction, or is not regulated at all. In the practice of the EA Rule, these regulation direction miscalls occur whenever the SCR does not equal one. However, as indicated in Table 7 (as well as Tables 4, 5, and 6), for a given SCR, a Regulation Direction Miscall (RDM) will occur only for genes which have a particular set of T-DGER values in the samples compared. The larger the difference between the total RNA content per cell, or mRNA content per cell, of the compared samples (that is the further the SCR deviates from one), the greater the range of gene T-DGER values which will fall into the RDM category. In a sample comparison where the EA Rule is practiced, the T-DGER range over which RDM's will occur is defined at one end by, (T-DGER=1), and at the other end by, (T-DGER)=(one÷SCR). The value of (one÷SCR) is equal to the ratio of, (the total RNA, or mRNA content per cell in one sample)÷(the total RNA, or mRNA content per cell in the other sample). Table 7 illustrates this. When the RNA content per cell for the samples compared differs by a factor of two (SCR=0.5), then the T-DGER range over which the RDM's occur is from (T-DGER=1), through about (T-DGER=2). In this case, the change in regulation direction will be miscalled for any gene in the sample comparison, which has a T-DGER in the samples of one through two. When the RNA content per cell for the samples compared differs by a factor of 10 (SCR=0.1), then the T-DGER range over which the RDM's occur is from (T-DGER=1), through about (T-DGER=10). In this case, the change in regulation direction will be miscalled for any gene in the sample comparison, which has a T-DGER of one through ten in the samples being compared. Table 1 indicates that the total RNA content per cell for adult rat liver is about 25 times greater than that for adult rat thymus. In the practice of the EA Rule the (SCR=0.04) with the thymus cells in the denominator. Here the N-DGER interpretation of the change in regulation direction will be miscalled for any gene in the liver-thymus comparison which has a T-DGER of one through twenty-five. The available information on the relative total RNA and mRNA contents of cells indicates that 2 to 10 fold differences are not uncommon. As mentioned earlier, 4 to 6 fold differences in total RNA or mRNA content per cell can exist for the same mammalian cells at different stages of growth. All prokaryotic and eukaryotic cells are associated with large differences in the RNA content per cell at different stages of the growth cycle.

TABLE 7
The T-DGER Range Over Which Regulation Direction Miscalls Occur in the
Practice of the EA Rule: Effect of SCR Value

| Relative Known | | | T-DGER | | | | | |
|---|---|---|---|---|---|---|---|---|
| Total RNA | | EA Rule | in Samples | Measured | Interpretation of | | | |
| Content Per Cell | | SCR | Compared | N-DGER | Regulation Direction | | | |
| G | NG | (G/NG) | (G/NG) | Must Equal | ^{(a)}N-DGER | | Reality | |
| 2 | 1 | 0.5 | 0.1 | 0.05 | D | 20x | D | 10x |
| | | 0.5 | 0.2 | 0.1 | D | 10x | D | 5x |
| | | 0.5 | 0.5 | 0.25 | D | 4x | D | 2x |
| | | 0.5 | 0.98 | 0.49 | D | 2.04x | D | 1.02x |
| | | 0.5 | 1 | 0.5 | D* | 2x | No Change | |
| | | 0.5 | 1.5 | 0.75 | D* | 1.33x | U | 1.5x |
| | | 0.5 | 1.96 | 0.98 | D* | 1.02x | U | 1.96x |
| | | 0.5 | 2 | 1 | *No change | | U | 2x |
| | | 0.5 | 2.02 | 1.01 | U | 1.01x | U | 2.02x |
| 10 | 1 | 0.1 | 10.1 | 1.01 | U | 1.01x | U | 10.1x |

| 0.1 | 10 | 1 | *No change | | U | 10x |
|---|---|---|---|---|---|---|
| 0.1 | 5 | 0.5 | D* | 2x | U | 5x |
| 0.1 | 2 | 0.2 | D* | 5x | U | 2x |
| 0.1 | 1 | 0.1 | D* | 10x | No change | |
| 0.1 | 0.98 | 0.098 | D | 1.02x | D | 10.2x |

*Gene Regulation Direction Miscalls
^{(a)}D = Down Regulated; U = Up regulated; x = Fold change in gene expression G = Growing Cells; NG = Non-growing Cells

As was discussed in the introduction, a typical mammalian cells' low abundance class mRNA contains thousands of genes which are expressed at a level of from 0.1 copy per cell to 5 to 10 copies per cell. In comparisons of different mammalian cell sample's low abundance mRNA populations, thousands of the same genes are expressed in both cell samples as low abundance mRNA's which are present in both cell samples at around one to five copies per cell. Consequently, in a mammalian cell comparison, thousands of genes represented in the low abundance mRNA class will have T-DGER values of between one and five. It seems highly likely that the practice of the EA Rule in a microarray comparison of mammalian cells will result in a large number of RDM's, even when the total RNA or mRNA content per cell of the compared samples differ by only two fold. For the liver-thymus comparison described above, it is likely that almost all EA related N-DGER will result in RDM's.

A similar situation occurs in yeast, where in a typical cell the low abundance, mRNA class represents several thousand expressed genes and the average number of mRNA transcript copies per cell is 1 to 2 (1). Here, a difference of two fold in the total RNA contents of the compared yeast cells could, in the microarray practice of the EA Rule, result in over half of the N-DGER being associated with RDM's. A difference of four fold in total RNA contents of the compared yeast cells could result in most N-DGER giving RDM's. Similar situations also exist for prokaryotes.

Non-microarray methods for gene expression analysis are commonly used to corroborate microarray gene expression results. Most, if not all of these alternative methods practice some form of the EA Rule. Therefore, the gene expression results obtained with these methods can but by no means always do, corroborate the microarray obtained results. The above discussion concerning the problem in interpreting EA Rule related microarray gene expression comparison results, also applies directly to gene expression results obtained by a non-microarray method of gene expression analysis which practices the EA Rule. This includes the methods of northern blotting, dot blots, nuclease protection, and RT-PCR, and the various forms of the differential display method. Here it is important to realize that it cannot be assumed that a result obtained by comparing purified mRNA can be corroborated by comparing the total RNA from the same samples, or vice versa. As an example, it cannot be assumed that a microarray result obtained by comparing sample cRNAs produced from T-RNA or isolated mRNA can be correctly corroborated by an RT-PCR result which produces the compared cDNAs from the same samples T-RNAs, as is often done. This is because the magnitude of the difference in total RNA content per cell between two samples is not necessarily equal to the difference in the total mRNA content per cell. Thus, depending on the situation the N-DGER ratio of the mRNA analysis may be significantly different from the N-DGER of the total RNA analysis for the same cell samples. In addition, under certain conditions, the total RNA analysis may yield a negative result for a gene with the total RNA analysis and a positive result for the same gene with a total mRNA analysis of the same samples.

Both microarray and non-microarray gene expression analysis assays have often used one or more housekeeping gene RNA's in order to control for experimental variables which are unrelated to any differences in gene expression which may exist in the samples being compared. A key requirement for the valid use of a housekeeping gene RNA for this control purpose, is that the level of the gene's expression of RNA must be the same in all compared samples. In this context, the level of the gene's expression of a RNA transcript often refers to the fraction of the total RNA, or total mRNA, which consist of a housekeeping gene RNA transcripts. The current, EA prior art Rule related experimentally based belief is that there are no housekeeping gene RNA's which are present at the same level in all samples which could be compared.

However, for the comparison of a limited number of particular samples it has been reported that particular housekeeping gene mRNA's are expressed to a similar level in these cell samples and can therefore be used as valid internal housekeeping standards. These results were obtained with the practice of the EA Rule. Because both of the above conclusions were obtained with the practice of the EA Rule, these conclusions may be erroneous. Absent knowledge of the actual sample cell ratios used in these microarray and non-microarray comparisons, the results are uninterpretable.

The above discussion applies directly to microarray and non-microarray methods of gene expression comparison analysis, including RT-PCR. The discussion has illustrated that differences in the number of RNA cell equivalents or CEs, which are directly compared in a microarray assay or non-microarray hybridization solution, or an RT-PCR assay amplification solution, is a global assay variable which is not taken into consideration by prior art microarray or non-microarray gene expression comparison analysis practice. The assay NF for this global assay variable is defined as the ratio of the number of RNA or cDNA, or cRNA, cell equivalents which are directly compared in the assay hybridization solution, or RT-PCR assay amplification solution. This NF is termed the sample cell ratio, or SCR. Note that the assay SCR value must be determined for the cell sample T-RNA, mRNA, or RNA equivalents which are directly compared and present in the assay hybridization solution, or the assay PCR amplification solution.

The invalidity of the first tacit assumption affects the assay SCR value so that under commonly occurring prior art assay conditions, the (N-DGER)=(ACR)≠(T-DGER). As a result, biologically inaccurate particular gene N-DGER values are determined for the assay. Note that for this discussion it has been assumed that for an assay, (N-DGER)=(NASR)=(ASR).

Retrospective Normalization of Prior Art Measured Particular Gene N-DGER Values for SCR. An Example.

Prior art gene expression comparison assay practice does not determine the assay SCR value, and does not normalize assay measured particular gene N-DGER values for the assay SCR value. In addition, information which can be used to retrospectively determine the SCR value for a published prior art gene expression comparison assay, is very rarely included in published reports, or otherwise available. An example of one of the very few instances, where a good estimate of the assay SCR value for a published gene expression comparison assay can be determined retrospectively from information in the report, and other information not present in the report, is described below. This retrospectively determined assay SCR value is used to normalize the prior art produced particular gene N-DGER values, and the effect of the use of this SCR value on the quantitative and qualitative characteristics of the published prior art assay measured and normalized particular gene N-DGER values is illustrated.

This prior art example involves a microarray gene expression comparison assay, which determines the genomic expression profiles of E. coli MG1655 rapidly growing (RG) cells from rich culture media, and slowly growing (SG) cells from minimal culture media. From these profiles, N-DGER values for expressed E. coli protein producing genes were obtained (143). This prior art example is discussed in great detail in the later section on the validity of prior art normalization assumptions.

One of the most comprehensively studied living organisms is the E. coli bacteria. Essentially all aspects of this bacteria have been extensively studied and documented, including the cell morphology, growth characteristics, genetics, biochemistry, and molecular biology. This includes the total RNA, mRNA, DNA, and protein contents, per cell for RG, as well as SG cells (10). It is well known that a RG E. coli cell contains much more T-RNA and mRNA than a SG cell, and that the actual T-RNA and mRNA contents per cell can be predicted from the growth rate or doubling time, of the bacterial cells (10). This is also true for other bacteria and other prokaryotes in general. It is known for example, that RG E. coli cells which have a doubling time of 25 minutes contain about 10 fold more T-RNA per cell and mRNA per cell, than do E. coli SG cells which have a doubling time of 57 minutes (10).

Pertinent experimental details obtained from the publication are summarized as follows. (i) E. coli MG1655 cultures were grown in batch culture in M63 minimal media, and Luria broth rich media, at 37° C. with aeration and shaking. Under these growth conditions the measured doubling times were, RG=25 minutes, SG=57 minutes. Here, the RG cells are known to contain 10 fold more T-RNA and mRNA on a per cell basis, than do the SG cells. (ii) T-RNA was quickly isolated and purified from RG and SG cells. Note that for this microarray assay, differences in the RNA isolation efficiencies for the RG and SG cell samples, have no effect on the assay SCR value. (iii) One microgram of RG T-RNA, and one microgram of SG T-RNA, were used to produce separate P³² labeled RG and SG cDNA preps for the assay. A specific gene primer for each of the 4290 E. coli genes examined was used to produce the cDNA preps. Care was taken to produce compared cDNAs with similar P³² specific radioactivities, and to compare similar total amounts of radioactivity for the RG and SG cDNA preps. This indicates that for this example, differences in the cell sample cDNA SE values have little effect on the assay SCR value. (iv) The entirety of each RG and SG cDNA prep was used in the assay hybridization step. (v) After hybridization and post hybridization processing, the assay signal associated with each gene spot was determined. Background was then subtracted from each gene spot signal. Duplicate spots were present for each gene, and the duplicate signal intensities for each gene were averaged for further analysis. (vi) For a compared microarray, each gene's spot signal intensity was expressed as a percentage of the total sum of all of the gene or spot signal intensities on the array. This is the widely used practice of total intensity normalization, or TIN, which prior art regards as a valid normalization method. (vii) The particular gene N-DGER values were obtained by comparing the averaged percent intensities for RG and SG genes. A particular gene assay measured N-DGER value is equal to, (the average percent signal intensity value for a particular gene on the SG array)÷(the average percent signal intensity value for the same particular gene on the RG array). Each particular gene N-DGER value was expressed as the log 10 of this ratio. (viii) A significant expression difference for a particular gene comparison in the assay is defined to occur when a difference in gene expression extent of 2.5 fold or greater occurs for a particular gene comparison.

For this published prior art example, the following is known. (a) RG cells contain 10 fold more T-RNA per cell than do SG cells. That is, the first tacit assumption is invalid for the assay. (b) The EA Rule is practiced for the assay. (c) N-DGER values are expressed in terms of the (SG/RG) ratio. The invalidity of the second tacit assumption will not affect the assay SCR value. (d) The third tacit assumption appears to be valid for the assay, or nearly so. (e) Because of items a-e, the SG/RG assay SCR value equals 10. (f) The published particular gene N-DGER values are not normalized for the assay SCR by the TIN process.

Tables 8 and 9 summarize the results of this comparison. These results were obtained from the publication and its supplementary material (www.ou.edu/microarray and 143). Of the 4290 protein producing E. Coli genes, which were included in the microarray assay, 3190 are detectably expressed in both the SG and RG cells. Of these, a very large number, 2846 genes, are unregulated, while 225 genes are upregulated in the SG cells, and 119 different genes are upregulated in the RG cells. These results are used in the report to categorize genes by functional grouping. The authors caution that a particular gene N-DGER ratio obtained from this comparison must be corroborated before being regarded as specific evidence for gene regulation, but indicate that the general trends represented by all of the results are substantially clear and useful.

TABLE 8
Gene Activity Budget For the E. coli SG Versus SG Comparison (143)

Activity of Genes In

| Number of Genes | SG Cells | RG Cells |
|---|---|---|
| 3190 | + | + |
| 96 | + | − |
| 307 | − | + |
| 697 | − | − |

TABLE 9
Summary of Prior Art Example Results (143)

| | | Prior Art | |
|---|---|---|---|
| | | (SG/RG) | |
| | | Particular Gene | |
| | Number of | N-DGER | ^{(a)}Prior Art |
| | Genes In | Values For | Interpretation of Gene |
| Gene Category | Category | Category | Expression Profile |
| Unregulated | 2846 | 0.4 to 2.5 | All 2846 Genes |
| Genes | | | Unregulated |
| Genes Active In | 225 | 2.51 to 74 | 225 Genes Significantly |
| SG and RG Cells | | | Upregulated In SG Cells |
| and Upregulated | | | (By 2.51 to 74 Fold) |
| In SG Cells | | | |
| Genes Active In | 119 | 0.39 to 0.1 | 119 Genes Significantly |
| SG and RG Cells | | | Upregulated In RG Cells |
| and Upregulated | | | (By 2.51 to 10 Fold) |
| In RG Cells | | | |

^{(a)}Assumes prior art N-DGER value of >2.5 or <0.4 is significant.

Table 10 presents a summary of these same results which have been normalized for the assay SCR value, which is equal to 10. This Table uses the same definition of significance for a ratio, as does the publication. That is a significantly expressed gene has an N-DGER value of <0.4 or >2.5. Note that here, the SCR is associated with a global assay variable, i.e., the natural differences in RNA content per cell for compared cell samples, and has only one assay value for all gene comparisons. The results of this normalization are quite striking. After SCR normalization 2846 prior art categorized unregulated genes, are significantly upregulated in the RG cells.

TABLE 10
Summary of SCR Normalized Prior Art Example
Normalized Gene Expression Results

| | | | | ^{(b)}SCR | |
|---|---|---|---|---|---|
| | Number | ^{(a)}Prior Art | | Normalized | ^{(c)}Interpretation of |
| Prior Art | of Genes | N-DGER | Assay | N-DGER | SCR Normalized |
| Gene | In | Values For | SCR | Values For | Gene Expression |
| Category | Category | Category | Value | Category | Profile |
| Unregulated | 2846 | 0.4 to 2.5 | 10 | 0.04 to 0.25 | All 2846 Genes |
| Genes | | | | | Significantly |
| | | | | | Upregulated In RG |
| | | | | | Cells (By 4 to 25 Fold) |
| Genes | 255 | 2.51 to 74 | 10 | 0.251 to 7.4 | 33 Genes |
| Active In | | | | | Unregulated |
| SG and RG | | | | | 186 Genes |
| Cells and | | | | | Significantly |
| Upregulated | | | | | Upregulated In RG |
| In SG Cells | | | | | Cells |
| | | | | | 6 Genes |
| | | | | | Significantly |
| | | | | | Upregulated In SG |
| | | | | | Cells |
| Genes | 119 | 0.39 to 0.1 | 10 | 0.039 to 0.01 | 119 Genes |
| Active In | | | | | Upregulated In RG |
| SG and RG | | | | | Cells (By 25 to 100 |
| Cells and | | | | | Fold) |
| Upregulated | | | | | |
| In RG Cells | | | | | |

^{(a)}All ratios are in terms of (SG/RG).
^{(b)}(Prior art N-DGER) ÷ (assay SCR) = (SCR normalized N-DGER).
^{(c)}Assumes SCR normalized N-DGER value of >2.5 or <0.4 is significant.

Before SCR normalization all 2846 of these genes were associated with erroneous N-DGER values, and regulation direction miscalls (RDMs). Further, 225 genes were prior art categorized as being upregulated in SG cells, and after SCR normalization about 186 of these genes are upregulated in RG cells, while about 33 of these genes are unregulated. Only about 6 of these 225 genes remain upregulated in the SG cells after SCR normalization. All 225 of these genes were associated with prior art measured and normalized N-DGER values which were erroneous by 10 fold, and 219 of these genes were associated with RDMs. The prior art categorized 119 genes, which were upregulated in RG cells, remained upregulated in RG cells after SCR normalization. All 119 genes were associated with N-DGER values, which were erroneous by 10 fold, but were not associated with RDMs. Overall then, before SCR normalization all of the 3190 genes which were expressed in both SG and RG cells were associated with assay measured N-DGER values which were erroneous by 10 fold, while 3065 of these genes were associated with RDMs. As a result of the SCR normalization, the interpretation of the general trends of the SCR normalized data is very different from the interpretation of the general trends of the published normalized data. In addition, the results from the data mining process of functionally grouping the expressed genes on the basis of the gene expression values, and the direction of regulation change implied by these N-DGER values, will be very different for the SCR normalized data than for the published data.

In addition to the erroneous N-DGER values and associated RDMs caused by not normalizing for the assay SCR, a significant number of the 307 genes which are expressed only in SG cells may be associated with false negative results which have occurred for these genes in the RG cells. Each such false negative result is associated with an RDM. Here, because of the assay SCR value, it is possible for the expression of a particular gene to be detected in SG cells and not in RG cells, even though the abundance of the particular gene mRNA in RG cells is equal to or greater than the mRNA abundance for the same gene in the SG cells. For an assay SCR value of 10, it is possible that the particular gene expression will be detected in SG cells, and not in RG cells, even though the particular gene mRNA abundance is 9 fold higher in the RG cells than the SG cells. The effect of the SCR on the occurrence of particular gene false negative values will be discussed in a later section.

Validity of the Relationship (N-DGER)=(ACR)=(T-DGER) when the Second Tacit Assumption is Invalid.

It is known that the cell sample RNA isolation efficiency is almost always significantly less than one, and that the RNA isolation efficiency values for different cell samples can vary significantly, depending on the condition and type of the cell sample (103). Prior art rarely determines the RNA isolation efficiency for the assay compared cell samples. In addition, little specific information is available regarding the isolation efficiencies of T-RNA and mRNA from cell samples, or the effect of different treatments on such efficiencies. Anecdotal and personal communication information suggests that it is not uncommon for the RNA isolation efficiency values of compared cell samples to differ by 2 to 3 fold or more.

It is very likely then, that the second tacit assumption is invalid for most prior art gene expression comparisons of all kinds. However, only a very small number of these prior art assays generate assay measured particular gene DGER values which can be caused to be biologically inaccurate by the invalidity of this assumption. This will be discussed below.

A small fraction of prior art gene expression comparison assays which practice the EA Rule is designed to determine particular gene N-DGER values by first determining for each compared cell sample a quantitative value for the number of particular gene mRNA transcripts per cell, or a quantitative value for the amount of assay signal activity per cell which is associated with a particular gene's mRNA transcripts or equivalents. The invalidity of the second tacit assumption for such an assay will cause these quantitative values to be biologically incorrect, and is likely to cause the N-DGER values derived from them to be biologically inaccurate. This is discussed below. For this discussion it is assumed that the first and third tacit assumptions are valid, the EA Rule is used, and that the invalidity of the second tacit assumption is the only assay variable which can cause the biological inaccuracy of the particular gene N-DGER values. For simplicity, the discussion will be presented in terms of particular gene mRNA transcripts per cell, or mRNA abundance. Such a prior art gene expression comparison assay is discussed below in terms of the following assay steps.
(a) The value for the amount of T-RNA or mRNA per cell is measured for each compared cell sample. For each compared cell sample, this value is determined by the standard prior art method of isolating and quantitating the amount of T-RNA or mRNA obtained from a known number of cells, and then determining the value for the amount of isolated T-RNA or mRNA per cell for each cell sample. (b) Equal amounts of isolated RNA from each cell sample is compared in the assay. (c) The known equal amount of cell sample isolated RNA used in the assay, is divided by the amount of isolated RNA per cell value determined for each cell sample. The result is the number of each cell sample's RNA cell equivalents (CEs) which are used in the assay. Herein, the ratio for the assay of (the number of RNA CEs for one cell sample)÷(the number of RNA CEs for the other compared cell sample), is termed the RNA CE number ratio, or RCNR. Here, since the first and third tacit assumptions are valid, and the EA Rule is used, the assay RCNR and SCR values will equal one if the second tacit assumption is also valid, and the assay measured N-DGER values will be biologically accurate. If the second tacit assumption is invalid, the RCNR and SCR assay values are not likely to equal one, and the N-DGER values are likely to be biologically inaccurate. (d) For each compared cell sample, the assay measured number of particular gene mRNA transcript molecules which is associated with the known amount of RNA used in the assay, is determined. (e) For each compared cell sample, the assay measured particular gene mRNA abundance value is determined, and is equal to, (the number of particular gene mRNA transcripts associated with the known amount of cell sample RNA used in the assay)÷(the calculated number of sample cell CEs for a cell sample which is associated with the known amount of cell sample RNA used in the assay). (f) A particular gene N-DGER value is then determined by comparing the particular gene mRNA abundance values for the compared cell samples.

For such an assay, when the second tacit assumption is invalid, the amount of RNA isolated from a known number of cells from either cell sample, is an underestimate of the actual amount of RNA present in the known number of cells. For each cell sample then, the value determined for the amount of T-RNA or mRNA per cell, is an underestimate. As a result, the calculated number of each cell sample's RNA CEs compared in the assay is inaccurate, and overestimated. In addition, because the prior art does not determine the RNA isolation efficiencies of the compared cell samples, the actual number of cell sample RNA CEs for each cell sample is unknown. Here, when the first and third assumptions are valid, and the EA Rule is practiced, when the RNA isolation efficiencies of the compared cell samples are the same, the assay (RCNR)=(SCR)=1. However, the RNA isolation efficiencies for different cell samples often vary significantly, and a difference in RNA isolation efficiencies of 2 fold or more, would not be surprising. When there is a significant difference in the compared cell sample RNA isolation efficiencies, the assay (RCNR)=(SCR)≠1. When the difference is 2 fold, then the assay SCR value is equal to either 0.5 or 2. For such an assay where the first and third assumptions are valid, the EA Rule is practiced, and the second tacit assumption is invalid, the assay SCR is, in essence, the only assay variable which can cause the assay N-DGER to be biologically incorrect. In this situation, an assay SCR value of 0.5 or 2 would cause the assay measured particular gene N-DGER values to be biologically inaccurate, and either over or under estimated by 2 fold.

Alternatively, a very small fraction of prior art gene expression comparison assays do not practice the EA Rule, but instead compare the RNA isolated from a known number of cells for each cell sample. Usually the entirety of the RNA isolated from each cell sample is compared in the assay. Such assays then determine a particular gene mRNA abundance value, or quantitative amount of particular gene assay signal activity per cell, for each compared cell sample. These values are then compared to obtain any assay measured particular gene N-DGER value. For such assays, the invalidity of the second tacit assumption can cause these particular gene N-DGER values to be biologically inaccurate. These prior art assays do not compare known, equal amounts of cell sample isolated RNA, but compare an amount of isolated RNA from each cell sample which is isolated from a known number of cells from each cell sample. The actual amount of isolated RNA compared, is often unknown. Such assays are designed by the prior art to measure a particular gene N-DGER value, by first determining for each compared cell sample, a quantitative value for the number of particular gene mRNA transcripts per cell, or a quantitative value for the amount of assay signal activity per cell which is associated with a particular gene's mRNA transcripts or equivalents which are put into the assay. The invalidity of the second tacit assumption will cause the measured value for the amount of particular gene mRNA in the cells to be biologically inaccurate and is likely to cause the particular gene N-DGER value derived from the quantitative values, to be biologically inaccurate. This is discussed below. For this discussion it will be assumed that the third tacit assumption is valid, and that the only assay variable which can affect the biological accuracy of the assay measured N-DGER values is the invalidity of the second assumption. Further, for simplicity this discussion will be in terms of the measurement of particular gene mRNA abundance values for compared cell samples, and the derivation of particular gene N-DGER values from them. Such a prior art gene expression comparison assay is discussed below in terms of the following assay steps. (a) The number of cells is determined for each cell sample. (b) For each cell sample, RNA is isolated from a known number of sample cells. The amount of RNA isolated may or may not be measured, and the RNA isolation efficiencies are not measured. (c) For each cell sample, an amount of RNA isolated from a known equal number of sample cells is compared in the assay. Here, the third tacit assumption is valid and the EA Rule may or may not be used, and if the second tacit assumption is valid, then the assay (RCNR)=(SCR)=1, and the assay measured N-DGER values will be biologically accurate. However, if the second tacit assumption is not valid, then the assay (RCNR)=(SCR)≠1, and the assay measured N-DGER values are likely to be biologically inaccurate. (d) For each compared cell sample, the assay measured number of particular gene mRNA transcripts associated with the amount of cell sample RNA used in the assay is determined. (e) For each compared cell sample, the assay measured particular gene mRNA abundance value is determined, and is equal to (the measured number of particular gene mRNA transcripts associated with the amount of cell sample RNA used in the assay)÷(the number of sample cells used to produce the amount of cell sample RNA used in the assay). Here, if the second tacit assumption is valid, then the particular gene mRNA abundance value is biologically accurate, because the amount of cell sample RNA used in the assay represents the entire amount of RNA present in the known number of sample cells used to isolate the RNA. However, if the second tacit assumption is not valid, then the particular gene mRNA abundance value will be biologically inaccurate, since the amount of cell sample RNA used in the assay, does not represent the entire amount of RNA present in the known number of sample cells used to isolate the RNA. Because the cell sample RNA isolation efficiency is less than one, only a portion of the RNA present in the known number of cells, is isolated. As a result, the number of cell sample RNA CEs which are used in the assay, is less than the number of sample cells used to isolate the amount of RNA use in the assay. (e) A particular gene assay measured N-DGER value is then determined by comparing the particular gene mRNA abundance values for the compared cell samples.

For such an assay, when the second tacit assumption is invalid, for each compared cell sample the number of cell sample RNA CEs which is used in the assay, is less than the number of sample cell RNA CEs used to determine the assay particular gene mRNA abundance values. The resulting assay mRNA abundance values are then, biologically incorrect and underestimated. In addition, because prior art does not determine the compared cell sample RNA isolation efficiencies, the actual assay RCNR and SCR value is unknown. Here, when the third assumption is valid, and the first assumption may or may not be valid, and the EA Rule may or my not be practiced, when the second assumption is valid then the assay (RCNR=(SCR)=1, and the assay N-DGER values are biologically correct. However, the RNA isolation efficiencies for different cell samples often vary significantly, and a difference in RNA isolation efficiencies of 2 fold or more, would not be surprising. When there is a significant difference in the compared cell RNA isolation efficiencies, the assay (RCNR)=(SCR)≠1. When the difference is 2 fold, then the assay SCR value is equal to either 0.5 or 2. For such an assay, the SCR is in essence, the only assay variable, which can cause the assay N-DGER values to be biologically incorrect. In this situation, an assay SCR value of 0.5 or 2 would cause the assay measured particular gene N-DGER values to be biologically inaccurate, and either over or under estimated by 2 fold.

For both assay examples discussed above the assay SCR value represents the assay normalization factor (NF), which is associated with multiple global assay variables. The global assay variables which directly influence the assay SCR value for a prior art gene expression analysis assay, are the validity for an assay of tacit assumptions one, two, and three.

Prior art examples of these assays which are affected by the validity of the second tacit assumption have been published (103, 144, 145, 146). These reports claim to have measured biologically accurate particular gene mRNA abundance values, or quantitative values for the amount of assay signal activity per cell which is associated with particular gene's mRNA transcripts or equivalents, and particular gene N-DGER values, for compared cell samples. However, as discussed, absent information not provided by these prior art reports, it cannot be known whether such assay results are biologically accurate or not. As an example, one report (144), indicates that gene expression comparison assay results were obtained using the isolated T-RNA from a known number of yeast cells. The known number of cells used for each cell sample, represented the number of viable yeast cells in the cell sample. However, no information was provided as to the fraction of each total yeast cell sample population, which consisted of viable cells. Therefore, while the value for the number of viable yeast cells which is associated with a known amount of yeast cell sample isolated T-RNA may be known, the value for the total number of yeast cells, both viable and quiescent, which is associated with a known amount of yeast cell sample isolated T-RNA cannot be known. Absent this information, it is not possible to determine biologically accurate particular gene mRNA abundance values, and N-DGER values. The report does claim to establish the validity for one yeast cell sample type, of the R and Fmole assumptions for particular gene mRNAs present in replicate, independently isolated T-RNA preps. In addition, this report does not determine the RNA isolation efficiency values for each analyzed or compared yeast cell sample.

In the context of the above discussion the second tacit assumption is pertinent for all microarray, non-microarray, and clone counting SGDS and DGDS gene mRNA transcript and all other cell sample RNA transcript type expression comparison assays, but is not pertinent for such DGSS assays.

Note that for this section on the validity of the prior art belief and practice that for an assay (N-DGER)=(ACR)=(T-DGER), it has been assumed that (N-DGER)=(ACR). The invalidity of the second tacit assumption affects the assay SCR value so that under commonly occurring prior art assay conditions, the (N-DGER)=(ACR)≠(T-DGER), and as a result, biologically inaccurate particular gene N-DGER values are determined.

Validity of Prior Art Relationship (N-DGER)=(ACR)=(T-DGER) when the Third Tacit Assumption is Invalid.

Prior art believes and practices that for a prior art SGDS microarray or non-microarray assay, the relationship (ACR)=(T-DGER) is true for a particular gene mRNA transcript comparison. Prior art further believes that when (ACR)=(T-DGER), then the assay measured particular gene (NASR)=(ACR)=(T-DGER), for the assay. By prior art definition, the (NASR)=(N-DGER) for a particular gene comparison. In order for the relationship (ACR)=(T-DGER) to be valid for assay compared cell sample cDNA preps, the number of compared cell sample cDNA cell equivalents (CE) must be the same for each cell sample. Prior art microarray and non-microarray assays practice the EA Rule and compare equal amounts of cell sample RNA in an assay, and also assume the validity of tacit assumption one for the assay. As a result, prior art believes that the amounts of each compared cell sample RNA put into the assay RT step represents the same number of cell sample RNA CEs. Thus, prior art believes that the ratio of the number of each compared cell sample's RNA CEs which is present in the assay RT step is equal to one for the assay. Prior art thereby assumes the third tacit assumption, and believes that the compared cDNA SE values are the same, and that the SER for the compared cell sample cDNA preps is also equal to one. In other words, that the assay compared cell sample cDNA prep SCR value is also equal to one for the assay. In this situation, the SCR will equal one only when the third tacit assumption is valid. For a particular gene comparison the relationship (ACR)=(T-DGER) is valid only when the assay value for the compared cell sample cDNA preps SCR is equal to one.

The third tacit assumption is pertinent for those microarray and non-microarray gene expression analysis assays, and gene expression comparison analysis assays, which directly compare cell sample cDNAs, but not those which directly compare cell sample cRNAs. The third tacit assumption for microarray assays which compare cell sample cDNA preps, indicates that in order for the particular gene assay relationship (ACR)=(T-DGER) to be valid, the compared cell sample cDNA SE values must be the same. For RT-PCR assays the third tacit assumption specifies that in order for the particular gene assay relationship (ACR)=(T-DGER) to be valid, the compared cell sample particular gene cDNA AE•SE values must be the same. For RT-PCR assays the third tacit assumption also concerns the compared particular gene assay ALGAE values. However, the particular gene comparison AE•AER assay value does not affect the validity of the relationship (ACR)=(T-DGER) for an assay or the assay particular gene comparison assay cDNA AE SCR value. The AE•AER assay value does affect the validity of the prior art belief that, (the assay measured NASR)=(ACR) for a particular gene comparison, and will be discussed later.

For the current discussion on the validity of the SE and AR•SE aspects of the third tacit assumption, the following will be assumed. Tacit assumptions one and two are valid. The R and Fmole assumptions are valid for each compared cell sample cDNA or cDNA AE prep. Each particular gene or standard ALGAE value is equal to one. The EA Rule is used for each SGDS cell sample mRNA transcript comparison assay. The relationship (N-DGER)=(NASR)=(ACR), is valid for each particular gene comparison. It is further assumed that only assay variable which can affect the validity of the prior art belief that (ACR)=(T-DGER), is the validity of the SE or AE•SE aspects of the third tacit assumption. Put differently, only the validity of the SE or AE•SE aspects of the third tacit assumption can cause the assay value for a particular gene comparison N-DGER or NASR or ACR, to deviate from the biologically accurate T-DGER value for the particular gene comparison.

It is highly likely that this third tacit assumption validity requirement is not met for many, if not most, microarray cDNA analysis assays, or RT-PCR assays. The reasons for this follow. It is known that for prior art microarray and RT-PCR assays the SE and AE•SE values for cell sample cDNA preps, particular gene cDNA preps, and standard cDNA preps are almost always equal to significantly less than one (103-106, 109-111, 147). While prior art does not measure the SE and AE•SE values for cell sample, particular gene, or standard cDNA preps, it does occasionally measure the ratio of, (the mass of cDNA produced in the RT step)÷(the mass of RNA template present in the RT step), which is herein termed the cDNA yield fraction or cDNA YF. The cDNA YF value for prior art microarray and RT-PCR assays is almost always equal to significantly less than one, and is generally around 0.1 to 0.5, and more usually around 0.1 to 0.3. It is also known that the cDNA YF values for cell sample, particular gene, and standard cDNA preps, can be affected by a variety of commonly occurring assay factors, and can vary significantly for different cell sample, particular gene, or standard cDNA preps. As a result, cDNA YF assay value differences of 1.5 to 2 fold or more for different microarray or RT-PCR assay analyzed cell sample particular gene, or standard cDNA preps or cDNA AE preps, would not be uncommon. This variability for the prior art microarray and RT-PCR assay cDNA YF values indicates that the prior art microarray and RT-PCR assay cell sample particular gene and standard cDNA SE and cDNA AE•SE assay values for different cell sample or particular gene or standard cDNA preps, also differ significantly and can differ by about the same amount as the cDNA YFs. Such cDNA SE or cDNA AE•SE values can differ by more than the cDNA YFs differ, or by less, depending on the characteristics of the synthesized cDNA. However, assay differences of 1.5 to 2 fold or more for the cDNA SE or cDNA AE•SE assay values for different assay compared cell samples, particular genes, or standards, would not be uncommon.

Prior art microarray and RT-PCR assay measured particular gene N-DGER values are believed by the prior art to be biologically accurate within the measurement accuracy of the assay. Prior art microarray and RT-PCR assay practice does not determine or normalize for the assay associated compared cell sample cDNA prep SCR values, or cDNA SER values, or cDNA AE•SER values. Therefore, in order to obtain a biologically accurate assay measured N-DGER value, prior art must assume that: (i) Each compared cell sample RNA in the assay RT step represents the same number of cell sample cell equivalents; (ii) Each assay compared cell sample cDNA prep or cDNA AE prep also represents the same number of cell sample cDNA CEs or ACEs, and the compared cDNA or cDNA AE assay SCR value equals one. The assay SCR value can equal one only when the third tacit assumption is valid and each compared cell sample cDNA SE or cDNA AE•SE value is the same. When the compared cell sample SEs or AE•SEs are significantly different, then the cDNA or cDNA AE SCR assay value deviates significantly from one, and the ACR value for each particular gene comparison deviates from the particular gene T-DGER value for the assay, and the relationship (ACR)=(T-DGER) is not valid. The magnitude of the SCR deviation from one, and the ACR deviation from the T-DGER, is then equal to the magnitude of the deviation of the compared cell sample SER assay value or AE•SER assay value, from one. In this situation, for an assay measured particular gene N-DGER or NASR value, the magnitude of the deviation from biological accuracy is also equal to the magnitude of the deviation of the compared cell sample SER or AE•SER assay values from one.

Prior art microarray and RT-PCR assays often claim a measurement accuracy of ±1.5 fold for prior art measured particular gene NASR and N-DGER values. For such an assay a deviation of the compared cell sample's cDNA SER or cDNA AE•SER value from one of ±1.5 or even ±1.2 fold can have a significant effect on the assay measured particular gene NASR and N-DGER values, and their prior art interpretation. As indicated above, it is very likely that compared cell sample cDNA and cDNA AE•SER values which deviate from one by ±1.5 fold to ±2 fold, are common for prior art microarray and RT-PCR assay practice. Prior art microarray practice does not determine cell sample comparison or particular gene comparison cDNA SER or cDNA AE•SER values, and prior art measured particular gene N-DGER values are not normalized for the SER and AE•SER. Absent such information it cannot be known whether the relationship (ACR)=(T-DGER) is valid for a prior art microarray or RT-PCR assay or not. However, it is very likely that the third tacit assumption is not valid for many, if not most, prior art microarray and RT-PCR assays.

Validity of Relationship (N-DGER)=(ACR)=(T-DGER) when Two or More Tacit Assumptions are Invalid.

The above discussions have indicated the following for prior art gene expression comparison assays. The first tacit assumption is often invalid for gene expression comparison assays of all kinds. The second tacit assumption is likely to be invalid for most gene expression comparison assays, which measure the number of mRNA transcripts per cell, or amount of assay signal activity per cell for a particular gene. Such assays comprise only a small fraction of the prior art assays. The third tacit assumption is likely to be invalid for most prior art gene expression comparison assays, which compare cell sample cDNAs. The vast majority of prior art gene expression comparison assays, which are done, utilize cDNA or cRNA. It is likely then, that many if not most, prior art gene expression comparison assays are associated with invalid assumptions one, two, and three.

Tacit assumption one is associated with natural differences in the amount of T-RNA or mRNA per cell which commonly occur for gene expression comparison assay compared cell samples. Tacit assumption two is associated with compared cell sample RNA isolation efficiencies. Tacit assumption three is associated with compared cell sample cDNA synthesis values. The invalidity of each of these assumptions causes the assay SCR value to deviate from one, and thereby causes the assay measured particular gene (N-DGER)≠(T-DGER), since the prior art does not determine or correct for the assay SCR value. Here the assay measured N-DGER deviates from the T-DGER, by the same magnitude as the SCR value deviates from one. The invalidity of each different tacit assumption has an independent effect on the assay SCR value. The aggregate effect of the invalidity of each of the assumptions for an assay, equals the product of the quantitative effect of each invalid assumption on the SCR value. The SCR value for an assay is then equal to, (the quantitative effect of the validity or invalidity of assumption one on the SCR)×(the quantitative effect of the validity or invalidity of assumption two on the SCR)×(the quantitative effect of the validity or invalidity of assumption three on the SCR). This can be illustrated by considering a gene expression comparison assay for which, all three tacit assumptions are invalid, the EA Rule is used, and there are no other assay variables which can affect the assay SCR value except the assumption invalidities. Practically, such an aggregate assay SCR value is relevant for prior art gene expression comparison assays, only if the assay SCR value deviates from one significantly. The illustration will address this issue. It is known that the intact cell RNA CE values commonly differ by as much as 4-10 fold or more, for different cell samples of the same cell type, and that differences of 2 to 4 fold are common. It is further known that the intact cell RNA CE values commonly differ by 2 to 25 fold or more for different cell types from the same organism, and that difference of 2 to 4 fold are common. Here, it's reasonable to believe that the intact cell RNA CE values for many prior art gene expression comparison assay compared cell samples, differ 3 fold. Such a difference will cause the assay SCR value to deviate from one by 3 fold.

It is also known that a cell sample RNA isolation efficiency is almost always significantly less than 1, and that the RNA isolation efficiencies for different cell samples often vary significantly, and RNA isolation efficiency differences of 2 fold or more, for compared cell samples would not be surprising. Here, it is reasonable to believe that the RNA isolation efficiency values for many prior art gene expression comparison assay compared cell samples, differ by 1.5 fold. Such a difference will cause the assay SCR value to deviate from one, by 1.5 fold.

It is further known that the cell sample SE value, which is associated with a microarray or non-microarray assay, is almost always equal to significantly less than one, and commonly ranges from 0.1 to 0.5. In addition, it is known that SE values for different cell samples commonly vary significantly, and SE differences of 3 fold would not be surprising. As a result, it is reasonable to believe that the SE values for many prior art microarray and non-microarray assay compared cell samples differ by 2 fold. Such a difference would cause the assay SCR value to deviate from one by 2 fold.

Each of the above derived estimates for the effect of the invalidity a tacit assumption on the assay SCR value is of a quantitative magnitude to have a very significant effect on the biological accuracy and interpretation of prior art assay measured particular gene N-DGER values. Many prior art assays report, and interpret, assay measured particular gene N-DGER values which deviate from one by ±1.5 to ±2 fold. These reported N-DGER values are not normalized for the assay SCR value. Further, the validity of the three tacit assumptions is not determined for these prior art assays. Many prior art assays claim to be able to obtain biologically accurate particular gene N-DGER values that are accurate to within ±1.2 to ±1.5 fold. The assays do not determine or correct for the assay SCR value. In this context, the estimated 1.5 fold effect of the invalidity of the second tacit assumption is highly meaningful and significant with regard to the biological accuracy and interpretation of prior art assay measured N-DGER values of all kinds. Note that each of these estimated quantitative effect values is believed to be a conservative estimate. It is believed that it would not be uncommon for each of these estimates, to be much larger for a prior art assay.

Table 11 illustrates the potential aggregate effect of these estimated values on a prior art gene expression comparison assay SCR value, and N-DGER value. Table 11 illustrates a situation where all three tacit assumptions are invalid, and pertinent to the assay. As discussed, it's likely that many prior art gene expression comparison assays are associated with the invalidity of all three of these assumptions, but for the vast majority of these assays, only the invalidity of assumptions one and three can have an effect on the assay SCR value, and are therefore, pertinent for the assay. As discussed, for only a small fraction of prior art assays, can the invalidity of the second tacit assumption affect the assay SCR value. Table 11 also illustrates that because each invalid assumption effect has an independent effect on the assay SCR value, then depending on the assay situation, the assay SCR value can be very different for these same three estimated effect values.

TABLE 11
Aggregate Effect of Invalidities of All Three Tacit
Assumptions On Assay SCR Value

| | | | | All Three | | All Assumptions | |
|---|---|---|---|---|---|---|---|
| | | | | Assumptions Invalid - | | Invalid. Only One | |
| | | | | All Are Pertinent | | and Three Are | |
| | | | | | ^{(c)}Deviation | Pertinent | |
| | | | | | of Assay N- | | ^{(d)}Deviation |
| | ^{(a)}Influence of Invalidity On | | | | DGER | | of N-DGER |
| | Assay SCR Value | | | ^{(b)}Assay | From | Assay | From |
| Assay | Tacit Assumption | | | SCR | Biological | SCR | Biological |
| Situation | One | Two | Three | Value | Accuracy | Value | Accuracy |
| (i) | ^{(a)}3 | 1.5 | 2 | 9 | 9 Fold | 6 | 6 Fold |
| (ii) | 3 | 1.5 | 0.5 | 2.25 | 2.25 Fold | 1.5 | 1.5 Fold |
| (iii) | 3 | 0.67 | 2 | 4 | 4 Fold | 6 | 6 Fold |
| (iv) | 3 | 0.67 | 0.5 | 1 | None | 1.5 | 1.5 Fold |
| (v) | ^{(a)}0.33 | 1.5 | 2 | 1 | None | 0.66 | 1.5 Fold |
| (vi) | 0.33 | 1.5 | 0.5 | 0.25 | 4 Fold | 0.165 | 6 Fold |
| (vii) | 0.33 | 0.67 | 2 | 0.45 | 2.2 Fold | 0.66 | 1.5 Fold |
| (viii) | 0.33 | 0.67 | 0.5 | 0.11 | 9 Fold | 0.165 | 6 Fold |

^{(a)}When the effect causes a 3 fold deviation from one, the quantitative value of the effect is either 0.33 or 3.
^{(b)}(Assay SCR value) = (effect of assumption one invalidity) × (effect of assumption two invalidity) × (effect of assumption three invalidity).
^{(c)}For this assay, the invalidity of only one of the tacit assumptions can affect the N-DGER value.
^{(d)}When the assay SCR <1, then the N-DGER value is underestimated relative to the T-DGER value.

For certain assay situations, the different effects interact to produce an assay SCR=1, and a biologically correct assay measured N-DGER values. For other assay situations, the different effects interact to produce an assay SCR=6 to 9, and assay N-DGER values which deviate from biological accuracy by 6 to 9 fold. In such a situation the actual assay N-DGER value could range from (0.11)×(T-DGER) to (9)×(T-DGER). Prior art does not determine the assay SCR value, and the prior art assay measured N-DGER values are not normalized for the assay SCR. Table 11 illustrates that absent such knowledge, prior art reported particular gene N-DGER values cannot be known to be biologically correct or not, and are therefore uninterpretable with regard to biological accuracy. However, many of these prior art assay measured N-DGER values have a high likelihood of being erroneous.

For a gene expression comparison microarray analysis, the natural differences in the compared cell sample's RNA CE values, the differences in compared cell sample's RNA isolation efficiency, and the differences in the cell sample's cDNA SE values, are each global assay variables. Consequently, an assay SCR acts as a global assay variable, whose value is influenced by the above-described differences. Each gene expression comparison assay is then associated with only one assay SCR value, and that SCR value applies equally to all particular gene assay measured DGER values in the assay.

It is clear that the aggregate effect of the invalidities of one or more of the tacit assumptions can cause the prior art believed and practiced relationship (N-DGER)=(ACR)=(T-DGER), to be invalid for many prior art gene expression comparison analysis assays.

Interpretation of Prior Art Measured N-DGER Values when the Assay SCR≠1.

Prior art gene expression comparison assay practice does not determine the assay SCR value and normalize the assay measured particular gene N-DGER values for SCR values, which deviate from one. Absent other compensating assay factors, an assay SCR≠1 value will cause the assay measured particular gene N-DGER values to be quantitatively inaccurate relative to the particular gene T-DGER values for the assay. In addition, an SCR≠1 assay value can also cause a regulation direction miscall (RDM) to occur for particular gene comparisons in the assay. An extensive discussion of the effect of SCR≠1 assay values on the quantitative value of assay measured N-DGER values was presented in the earlier section on “The validity of the relationship (N-DGER)=(T-DGER) when the first tacit assumption is invalid.” Included in this discussion is the effect of assay SCR≠1 values on the occurrence of RDMs for particular gene comparisons. These discussions are directly applicable to assay SCR≠1 values caused by the invalidity of any of the tacit assumptions.

Effect of the Validity of the Prior Art Belief and Practice that Essentially all mRNA Transcripts in a Eukaryotic Cell Possess Significant Poly a Tracts, on the Relationship (N-DGER)=(ACR)=(T-DGER).

For this discussion, the following will be assumed for a gene expression comparison assay. (i) For a particular gene comparison, (N-DGER)=(NASR)=(ACR). (ii) The EA Rule is practiced. (iii) The aggregate effect of the validity or invalidity of assumptions one, two, and three, produces an assay SCR=1.

Most prior art microarray and non-microarray gene expression analyzes compare the purified PA mRNA molecule populations prepared from the compared cell samples. Each such purified PA mRNA is isolated from the separate cell sample's total RNA by oligo dT binding affinity purification. This purification method will isolate PA mRNA molecules which have a PA tract of significant length, that is a PA tract which is long enough to stably bind to oligo dT. Such a PA tract is usually longer than about 15-20 nucleotides. Prior art generally believes and practices that such an isolated PA mRNA preparation represents essentially the total mRNA population of the cell or cell sample, and that only a small fraction of each particular gene's cell mRNA does not stably bind to oligo dT. Here the non-binding mRNA is termed PA⁻ mRNA. If this belief is correct then each different gene mRNA molecule population in a cell is composed of almost exclusively PA mRNA molecules, which can be isolated by oligo dT binding. This results in being able to compare for any particular gene in an assay, all of the gene's mRNA molecules which are present in one cell sample, to all of the same gene's mRNA molecules which are present in another cell sample. This belief and practice greatly simplifies the interpretation of the prior art gene expression comparison results. This occurs because it is not necessary to correct or normalize the assay results for the fraction of the total mRNA of a cell sample, which is comprised of PA mRNA.

It is generally believed that virtually all eukaryotic mRNAs possess a significantly long PA tract early in their lifetime. It is known that the PA tract length is often greatly shortened over the lifetime of many RNA types (148, 149, 150). Specific mammalian mRNAs that are deadenylated in the cytoplasm and accumulate to a large extent as PA⁻ mRNAs, have been reported (149, 150). After deadenylation these mRNAs did not bind to oligo dT. Another report indicated that one particular mRNA type, which possessed a significantly long PA tract, could not be isolated by oligo dT binding because the PA tract was unavailable for binding. Other reports indicate that certain mammalian mRNA types possess a spectrum of short PA tract lengths, some of which were long enough to bind to oligo dT, while others of the same type could not. Further, it has been reported for yeast that a large fraction (25-50 percent) of the total cell mRNA, can exit in the PA⁻ form in the cell. It was not reported whether all different mRNA type population in the yeast cell had the same proportion of PA mRNA, or whether some particular mRNA type populations were comprised of a higher proportion of PA mRNA than others.

These observations suggest that the ratio in a cell for a particular mRNA of, (oligo dT bindable mRNA)÷(total mRNA), can vary significantly for many different mRNA molecule types in the same cell. It also raises the possibility that for any particular mRNA in a cell, the ratio will vary under different cell conditions, such as cell cycle, cell growth, cell age, cell differentiation, cell size, chemical treatment, and physical treatment.

The above discussion indicates that the prior art belief and practice that the large majority of each different cell mRNA type possesses a PA tract which can bind stably to oligo dT, is often not valid for particular mRNA types in a cell, and in one case a large fraction of the mRNA types in a cell. Overall, for mammalian cells, specific knowledge concerning this assumption is limited to a relatively small number of different mRNA types. However, it is likely that many particular gene mRNAs are associated with significant fractions of PA⁻ mRNA. The effect of this situation on microarray and non-microarray assay results, and their interpretation is discussed below.

The above observations indicate that for a particular gene mRNA transcript in a cell, the ratio of, (the number of particular gene mRNA transcripts which can stably bind to poly dT or poly U)÷(the total number of particular gene mRNA transcripts present in the cell), can deviate significantly from one for many different mRNA types. Herein, such a ratio for a particular gene's mRNA in a cell or cell sample, is termed the PA Fraction, or PAF, for the particular mRNA in the cell. In different cell samples the PAF value for a particular gene mRNA in one cell sample, may be significantly different than the same gene mRNA PAF value in another cell sample. Herein, for such a particular gene mRNA, the ratio of (the PAF value for one cell sample)÷(the PAF value for a compared cell sample), is termed the PAF ratio, or PAFR, for the particular gene mRNA in the cell sample comparison. For certain microarray or non-microarray cell comparison assays, when the assay PAFR value for a particular gene mRNA deviates significantly from one, then a biologically correct gene expression level ratio for the gene cannot be obtained, unless the assay result for the particular gene comparison is normalized for the difference in the cell sample gene mRNA PAF values. The particular gene mRNA comparison assay result can be normalized for the assay variable associated with the cell sample PAF values, by dividing the particular gene mRNA comparison RASR value by the PAFR value associated with the gene mRNA comparison. This PAFR value represents the assay variable NF associated with the PAF related assay variable. Since the assay PAFR values for different gene mRNAs in the same cell comparison assay can differ significantly, the PAF related assay variable is a non-global assay variable, and the PAFR is a non-global assay variable NF.

The PAF related assay variable is not relevant to all prior art microarray and non-microarray cell sample gene comparison assays. It is relevant only to those microarray or non-microarray assays which directly compare: Isolated cell sample PA mRNA molecule preparations, or their cDNA or cRNA equivalents; mRNA molecules which have the signal label attached directly to the PA portion of the mRNA; labeled cDNA or cRNA molecules which require the PA tract of the mRNA in order to produce the labeled mRNA derived polynucleotides. The PAF related assay variable is not relevant to those microarray and non-microarray assays which directly compare: unpurified mRNAs present in the compared cell samples total RNAs; labeled cDNA or cRNAs which are derived from the unpurified mRNA present in the compared cell sample total RNAs, and which do not require the presence of a PA tract for labeling. For these latter assays, the PAFR assay NF value is always equal to one, and therefore there are no PAF differences to normalize for. Practically, this means that the PAF related assay variable may be relevant to any assay which compares mRNA LPN preparations produced by oligo dT priming of a labeling reaction, or which compares mRNA LPN preparations produced by random priming of purified mRNA.

The effect of the PAF related assay variable on the microarray and or non-microarray assay relationship (N-DGER)=(ACR)=(T-DGER) for a particular gene comparison, is illustrated in Table 12. Table 12 illustrates the effect of the PAFR on the assay ACR and RASR, when the PAFR is the only assay variable which is pertinent to the assay. For this illustration it has been assumed that the assay SCR=1, and that the relationship (N-DGER)=(NASR)=(ACR), is true for each particular gene comparison in the assay, and that oligo dT binding was used to isolate the assay compared PA mRNA preparations. Table 12 indicates that when the PAFR value for a particular gene comparison deviates from one, the N-DGER deviates from the T-DGER for the particular gene comparison by the same magnitude.

TABLE 12
Effect of PAF Related Assay Variable On the Relationship (N-DGER) =
(T-DGER) For A Particular Gene Comparison In An Assay

| | | | | | | | | Resulting | Prior Art |
|---|---|---|---|---|---|---|---|---|---|
| | | | | Gene | | | Resulting | Gene | Interpretation |
| Cell | | | ^{(a)}Gene's | mRNA | Assay | Assay | Gene | Assay | of Gene N- |
| Sample | | Gene | T-DGER | PAF | PAFR | SCR | ACR | N-DGER | DGER |
| (i) | 1 | A | 1 | 1 | 1 | 1 | 1 | 1 | Unregulated |
| | 2 | A | | 1 | | | | | |
| (ii) | 1 | A | 1 | 0.5 | 1 | 1 | 1 | 1 | Unregulated |
| | 2 | | | 0.5 | | | | | |
| (iii) | 1 | A | 1 | 0.5 | 0.5 | 1 | 0.5 | 0.5 | Down 2x^{(b)} |
| | 2 | | | 1 | | | | | |
| (iv) | 1 | B | 1 | 1 | 2 | 1 | 2 | 2 | Up 2x^{(b)} |
| | 2 | | | 0.5 | | | | | |
| (v) | 1 | C | 2 | 0.2 | 0.25 | 1 | 0.25 | 0.25 | Down 4x^{(b)} |
| | 2 | | | 0.8 | | | | | |
| (vi) | 1 | D | 100 | 0.5 | 0.5 | 1 | 50 | 50 | Up 50x |
| | 2 | | | 1 | | | | | |
| (vii) | 1 | E | 1 | 0.5 | 0.5 | 2 | 1 | 1 | Unregulated^{(a)} |
| | 2 | | | 1 | | | | | |
| (viii) | 1 | F | 1 | 0.5 | 0.5 | 0.5 | 0.25 | 0.25 | Down 4x^{(b)} |
| | 2 | | | 1 | | | | | |

^{(a)}All ratios involve (cell sample 1 parameter) ÷ (cell sample 2 parameter).
^{(b)}Regulation Direction Miscall (RDM).

Further, Table 12 indicates that when the PAFR value for a particular gene comparison deviates from one, a regulation direction miscall (RDM) can occur. The characteristics of the PAFR related RDMs are quite similar to the characteristics of the SCR related RDMs which were discussed extensively earlier. Note however, that the SCR NF is associated with a global assay variable, while the PAFR NF is associated with a non-global assay variable. Because of this, different particular gene comparisons in the same assay can have different PAFR values. The consequence of this is illustrated in Table 12 (iii) and (iv). Here, both genes A and B have a T-DGER=1. However, because the PAFR values are different for each gene, gene A appears to be downregulated 2 fold in cell sample 1, while gene B appears to be upregulated 2 fold in cell sample 2, even though both genes are in reality, unregulated. As can any other global or non-global assay variable, the PAF related assay variable can cause the occurrence of PAF related false negative assay results for particular gene comparisons.

Prior art microarray practice often compares cell sample isolated mRNA derived labeled cDNA or cRNA LPNs in a microarray assay, and then compares unfractionated cell sample total RNA in the northern blot, dot blot, nuclease protection, or RT-PCR method used to corroborate particular gene comparison microarray results. Here the PAF related assay variable can be associated with any particular microarray assay gene comparison. In contrast, the PAF related assay variable is not pertinent to any particular gene comparison in the corroborative assay. In this situation, the corroborative assay gene expression level ratio result may be greater than that for the microarray result.

Clearly the PAF related assay variable can cause the relationship (N-DGER)=(ACR)=(T-DGER) to be invalid for particular gene comparisons in a prior art microarray or non-microarray gene comparison assay. How often this has occurred for particular gene comparisons in prior art microarray or non-microarray gene expression analysis, is unknown. Prior art does not determine and take into consideration the PAFR for particular gene comparisons in the prior art normalization process. Absent such knowledge, for those prior art microarray and non-microarray assays which utilize only PA mRNA to produce the compared mRNA LPN preps, it cannot be known whether the relationship (ACR)=(T-DGER) is valid or not for any particular gene comparison, or whether the assay gene expression level ratio in biologically correct or not.

Note that most clone counting methods analyze only the PA mRNA fraction from the cell sample T-RNA. Therefore, the PAFR UNF is pertinent for all clone counting method particular gene comparisons.

Aggregate Effect on the Biological Accuracy of a Particular Gene N-DGER Value of the assay values for SCR≠1, and PAFR≠1.

The effect of the SCR and the PAFR on the assay measured N-DGER value, are independent of each other. Further, SCR is a global assay variable, and as such there is only one SCR value for an assay, and each particular gene N-DGER is affected to an equal extent by the SCR. In contrast, PAFR is a non-global assay variable for an assay, and as such there can be multiple different PAFR values for an assay, and each different PAFR value is associated with only one particular gene or one subset of particular genes. For those particular genes in an assay which are associated with an SCR≠1, and a PAFR≠1, then the aggregate effect on the N-DGER value, and on the deviation of the N-DGER from biological accuracy, is equal to, (assay SCR value)×(assay PAFR value). This is illustrated in Table 12 (viii), where the (PAFR=0.5) and the (SCR=0.5). Here, even though the T-DGER=1 for particular gene F, the N-DGER value is equal to (0.5×0.5) or 0.25. Table 12 (vii) illustrates that the SCR and PAFR assay values cancel each other out to produce a biologically correct N-DGER value. Note that when the aggregate effect equals the product of (the global assay variable SCR)×(the non-global assay variable PAFR), the resulting aggregate normalization product of (a global assay variable SCR≠1)×(a non-global assay variable PAFR≠1), then the resulting aggregate NF value is non-global in nature.

It is not clear whether PAFR values are common for prior art assays or not. However even small deviations of the assay PAFR values from one, can have a significant effect on the biological accuracy of a particular gene N-DGER, when combined with an assay SCR value which deviates from one by a small amount. A PAFR value of 0.75 for a particular gene, combined with an SCR value of 0.67 for the assay, gives an aggregate value of about 0.5, a twofold deviation from one. Absent other compensating factors, an aggregate value of 0.5 would cause the particular gene N-DGER value to deviate from biological accuracy by twofold. For a prior art assay, which claims an accuracy of measurement of the N-DGER of ±1.5 to 2 fold, as many prior art assays do, this aggregate twofold effect is highly significant.

Summary: Validity of the Relationship (N-DGER)=(ACR)=(T-DGER) for Prior Art Microarray and Non-Microarray Gene Expression Comparison Assays.

Prior art gene expression comparison practice assay measured particular gene N-DGER values are not normalized for the assay SCR value. The invalidity of one or more of the three prior art believed and practiced tacit assumptions, can affect the assay SCR value, and cause it to deviate from the value of one. Prior art does not determine the invalidity of these three assumptions, or determine or know, the assay SCR values for prior art gene expression comparison assays. It is highly likely that one or more of the three tacit assumptions is invalid for most prior art gene expression comparison assays, and that the assay SCR values for many of these prior art assays deviates significantly from one. Absent compensating assay factors, these assay SCR≠1 values will result in biologically incorrect prior art produced particular gene N-DGER values. In other words, for many prior art gene expression comparison assays the (N-DGER)=(ACR)=(T-DGER) relationship is invalid. Many of these biologically incorrect prior art N-DGER values will be associated with RDMs. The invalidity of this relationship can cause the occurrence of numerous EA Rule or SCR related, false negative particular gene expression results, and their associated RDMs.

Natural differences in the PAF values for particular mRNAs in compared cell samples, coupled with prior art assay practices, can result in assay PAFR not equal to one values for particular gene comparisons in the assay, which deviate significantly from one. These PAFR values will cause the assay measured N-DGER values for the particular genes to be biologically incorrect. In other words, for these prior art particular gene comparisons, the (N-DGER)=(ACR)=(T-DGER) relationship is invalid. Many of these biologically inaccurate particular gene N-DGER values will be associated with RDMs. Further, the invalidity of this relation can also cause the occurrence of numerous PAFR related false negative results and their associated RDMs. Prior art gene expression comparison practice assay measured particular gene N-DGER values, are not normalized for particular gene assay PAFR values. Prior art does not determine, or know, the particular gene PAFR assay values.

Prior art does not determine, or normalize gene expression comparison assay produced particular gene N-DGER values for, the assay SCR values, or particular gene assay PAFR values. Because of this, it is highly likely that many prior art assay measured particular gene N-DGER values are biologically inaccurate. However, absent knowledge not provided by the prior art, it cannot be known whether any particular prior art produced particular gene N-DGER values is biologically correct or not, and therefore all such prior art particular gene N-DGER values are uninterpretable with regard to biological accuracy. This includes particular gene N-DGER values used to corroborate particular gene N-DGER results. In other words, absent certain information which is not available, it cannot be known whether the relationship (N-DGER)=(ACR)=(T-DGER), is valid or not. However, as discussed earlier, a prior art produced positive result for a particular gene can be interpreted in a biologically accurate manner as being expressed in the cell sample being assayed.

Prior art produced particular gene mRNA expression analysis assay results for one or more cell samples, and gene expression comparison assay produced particular gene N-DGER values for compared cell samples, is frequently used for data mining analysis. Such data mining analyzes include scatter plots, principle component analysis, expression maps, pathway analysis, cluster analysis, self-organising maps and others (7, 34). Because of the above discussed biological inaccuracy of most prior art measured particular gene quantitative mRNA expression extents, the likely biological inaccuracy of many if not most, prior art gene expression comparison assay particular gene quantitative N-DGER values, and because these N-DGER values cannot be known to be correct or incorrect and are therefore uninterpretable with regard to biological accuracy, their use in data mining analysis is problematic.

Validity of Prior Art Assumptions Required for the Accuracy of Prior Art Clone Counting Method Measured Particular Gene mF and mFR Values.

Prior art believes and practices that a clone counting measured particular gene mF value for a cell sample is equal to the ratio of, (the number of particular gene mRNA molecules present in the intact cell sample)÷(the total number of mRNA molecules of all kinds in the intact cell sample), which is here termed the particular gene mRNA mF. In order for such belief and practice to be valid for the cell sample cloned tag library, the earlier discussed R and Fmole assumptions must be valid for the clone counting method pertinent portion of each mRNA molecule of any kind which is present in the intact cells of the analyzed cell sample. Thus, for such an analysis, the R and Fmole assumptions must be valid for the isolated cell sample T-RNA or mRNA, the cell sample cDNA prep produced from the cell sample RNA, and the cell sample mRNA tag clone library produced from the cell sample cDNA, for at least the clone counting method pertinent portion of each different mRNA molecule of any kind which is present in the intact sample cells. An earlier section concluded that for cell sample oligo dT primed cDNA preps the R and Fmole assumptions appear to be valid for the 3′ end of cell sample mRNAs which are associated with Poly A tracts. Whether the R and Fmole assumptions are valid for a cell sample mRNA tag clone library produced from the cell sample cDNA prep is not known. However, prior art widely assumes that such assumptions are valid for such a library. Note that the PAFR is pertinent to clone counting method assays.

Prior art believes and practices that an assay measured biologically accurate particular gene abundance value for a cell sample, can be determined by multiplying a clone counting method measured particular gene mF value by an estimated or measured value for the total number of mRNA molecules of all kinds per sample cell. Here the total number of RNA molecules of all kinds per sample cell value is termed the sample total mRNA value or, STM. The STM value used by the prior art for the particular gene mRNA abundance determination, is a commonly an estimated STM value, which is assumed to be the same for different cell types. As an example, prior art commonly estimates that the STM value for a typical mammalian cell is 300,000 mRNA transcripts per cell, while the STM for a typical yeast cell is 15,000 mRNA molecules per cell. In order for such belief and practice to be valid, different cell types must have the same STM values, and the STM value must be known. It is well known that different cell samples can, and often do, have significantly different STM values. As discussed earlier, the STM values for a bacterial cell can vary by as much as 10 fold depending on its growth rate, while the STM value associated with a rapidly growing cultured mammalian cell sample is about six times larger than the STM for slowly growing cells. In addition, the STM values associated with different cell types in the same mammalian organism can vary greatly, and potentially can vary by twenty fold or more. Clearly then, the prior art use of the estimated STM values to determine the abundance value for a particular gene from the SAGE measured particular gene mF value is not appropriate, unless it is known that the estimated STM value is accurate for the SAGE cell sample comparison. Further, in order to determine the cell sample STM value by using prior art practices, the earlier discussed second tacit assumption must be valid, or the isolation efficiency of the cell sample T-RNA and mRNA must be known. Prior art clone counting method practice does not determine or know the cell sample mRNA isolation efficiency, or determine or know the cell sample STM value. Therefore, the use of the estimated STM value to determine a particular gene abundance value from a SAGE measured particular gene mF value, is invalid for many such prior art produced particular gene abundance values, and cannot be known to be valid for other such values.

Prior art believes and practices that a clone counting method measured particular gene comparison mFR value is equal to the particular gene T-DGER value which exists in the compared cell samples. In order for such belief and practice to be valid, the first tacit assumption must be valid, and each compared cell sample must have the same STM value. As discussed extensively earlier, it is well known that the STM values for compared cell samples often vary significantly, by up to 2-10 fold or more, and prior art practice does not determine the STM values for each compared cell sample.

For a cell sample comparison the ratio of the compared cell sample's STM values is termed the STM ratio, or STMR. When for a cell sample comparison the STMR=1 a measured particular gene mFR is biologically accurate, and the (particular gene T-DGER value)=(the particular gene mFR value). When the STMR≠1, then the (T-DGER)≠(mFR) for the particular gene comparison. Further, when the STMR≠1, then the (particular gene T-DGER)=(particular gene mFR)×(STMR). This can be illustrated by considering the following. (a) Cell samples X and Y are analyzed. (b) The STM values for the compared cell samples are 9×10⁵ mRNA molecules per sample X cell, and 3×10⁵ mRNA molecules per sample Y cell. (c) For the compared cell samples, particular gene T has an mRNA abundance value of 9 copies per X cell and 3 copies per Y cell. (d) The particular gene T mRNA mF which exists in each sample cell is 10⁻⁵ for both cell samples X and Y. Here, (the T mRNA mF for cell sample X)=(9 T mRNA copies per X cell)÷(9×10⁵ total mRNAs for an X cell)=10⁻⁵, and for the Y cell sample (the T mRNA mF)=(3 T mRNA copies per Y cell)÷(3×10⁵ total mRNAs for a Y cell)=10⁻⁵. (e) The clone counting method analysis is done on each cell sample tag clone library, and the measured particular gene T mF values obtained are

10⁻⁵ for both cell samples X and Y. These mF values are biologically accurate. (f) The SAGE measured particular gene T mFR value is equal to one. This measured particular gene T mFR value is also biologically accurate. (g) Prior art believes and practices that the clone counting method measured particular gene mFR value is equal to the T-DGER value for the particular gene comparison. The prior art interpretation of this clone counting measured particular gene T mFR value, is that for this comparison, gene T mRNA is unregulated. This is not a biologically accurate interpretation, since it is known that the T gene is upregulated threefold in cell sample X. (h) Here, the (STM=3) assay value causes the prior art interpretation of the gene T mFR value to be erroneous with regard to the quantitative difference in the extent of gene T expression in the compared cell samples, but also causes a regulation direction miscall (RDM), which indicates that the gene T is unregulated when in reality it is 3 fold upregulated in cell sample X. (i) This example assumes that either, the cell counting method assay analysis has worked perfectly and the cell sample STMR value is the only assay variable which can affect the biological accuracy of the mFR, or that the gene T mFR value has been normalized for all pertinent prior art considered normalization factors.

Prior art practice does not determine the STM values for values for clone counting method analyzed cell samples, or the STMR values for clone counting method analyzed cell sample comparisons, and it is known that the STMR values for such prior art cell sample comparisons often deviate significantly from one. As a result, for any particular prior art clone counting method cell sample comparison assay, it cannot be known whether the STMR value equals one or not. Therefore, the prior art particular gene mFR values associated with such prior art cell sample comparisons are uninterpretable with regard to quantitative value and direction of gene expression regulation change. Note that the STMR is a prior art unconsidered assay variable normalization factor (UNF), and is a global UNF.

Table 13 illustrates further the effect of the assay STMR value on the prior art interpretation of clone counting method measured particular gene mFR values. The illustration involves the comparison of the earlier described growing (G) and non-growing (NG) cultured mammalian 3T3 cells from mouse.

Clone Counting Method Assay

TABLE 13
Comparison of Growing (G) and Non-Growing (NG) 3T3
Cells. Measured mFR Relationship to T-DGE

| Gene T | | | | | | |
|---|---|---|---|---|---|---|
| mRNA | | Clone Counting | | | Interpretation of Regulation | |
| Transcripts | | Method | | Assay | Direction of Growing Gene | |
| Per Cell | | Measured T mF | | Measured T | Activity | |
| G | NG | G | NG | mFR | Prior Art | Reality |
| 0.1 | 1 | 1.6 × 10⁻⁷ | 10⁻⁵ | 0.0167 | Down 60x | Down 10x |
| 1 | 1 | 1.67 × 10⁻⁶ | 10⁻⁵ | 0.167 | *^{(a)}Down 6x | No Change |
| 2 | 1 | 3.34 × 10⁻⁶ | 10⁻⁵ | 0.334 | *Down 3x | Up 2x |
| 5 | 1 | 8.35 × 10⁻⁶ | 10⁻⁵ | 0.835 | *Down 1.2x | Up 5x |
| 5.9 | 1 | 9.9 × 10⁻⁶ | 10⁻⁵ | 0.99 | *Down 1.01x | Up 5.9x |
| 6 | 1 | 10⁻⁵ | 10⁻⁵ | 1 | *No Change | Up 6x |
| 10 | 1 | 1.67 × 10⁻⁵ | 10⁻⁵ | 1.67 | ^{(a)}Up 1.67x | Up 10x |
| 100 | 1 | 1.67 × 10⁻⁴ | 10⁻⁵ | 16.7 | Up 16.7x | Up 100x |
| 1,000 | 1 | 1.67 × 10⁻³ | 10⁻⁵ | 167 | Up 167x | Up 1,000x |

*Gene Activity Regulation Direction Miscalls (RDM)
^{(a)}D—Downregulated; U—Upregulated: xFold Change in Gene Expression
^{(b)}Growing (G) Cell STM = 6 × 10⁵ mRNA Transcripts Per Cell Non-Growing (NG) Cell STM = 10⁵ mRNA Transcripts Per Cell (G/NG) STMR = 6

It is known that the STM value for G 3T3 cells is 6 times greater than the NG 3T3 cell STM value, and the STMR value for a 3T3 (G/NG) cell sample comparison is equal to 6. For this illustration, it has been assumed that the STM values are 6×10⁵ mRNA transcripts per cell for G cells, and 1×10⁵ mRNA transcripts per cell for NG cells. Further, in order to illustrate the effect of the interaction of the T-DGER and STMR values for a particular gene comparison, different particular gene T-DGER values are examined. For this illustration the assay STMR value is the only pertinent assay variable. The effect of the assay STMR value on the prior art interpretation of a clone counting method measured particular gene mFR value is very similar to the earlier extensively discussed effect of the microarray assay SCR value on particular gene N-DGER values. The greater the deviation of the assay STMR from one, the greater the range of particular gene T-DGER values which will give RDMs. For this cell sample comparison, the T-DGER value range in the assay over which RDMs will occur is defined at one end by about T-DGER=1, and at the other end by about T-DGER=6. Therefore, for the Table 13 illustration, the T-DGER range over which RDMs will occur for any particular gene in the assay which has a T-DGER value of 1-6. Note that in a typical prokaryote or eukaryote cell sample comparison analysis, a large fraction of the expressed particular genes have T-DGER values of 1-6.

Application of the Validity Discussions to the Gene Expression Analysis Assays of All Kinds.

The above discussions on the validity of the prior art belief and practice that for a particular gene comparison assay, the relationship (N-DGER)=(ACR)=(T-DGER) is valid. These discussions were primarily in the context of SGDS comparisons of particular gene mRNA transcripts. However these discussions are directly applicable to SGDS, and DGDS, assay analyzes of viral, prokaryotic, eukaryotic, and synthetic RNA types of all kinds, including all types and kinds of rRNAs, tRNAs, mRNAs, siRNAs, miRNAs, snoRNAs, antisense RNAs, and other known or unknown RNAs which occur in a cell. Note that for clone counting method DGSS particular gene comparisons, the STMR is not pertinent.

D. Validity of Prior Art Belief that (Nasr=Acr) for A Particular Gene Comparison

In the previous section, which discussed the validity of the prior art belief that for a particular gene comparison (assay NASR)=(assay ACR)=(T-DGER), it was assumed that the prior art belief that for a particular gene comparison, the (assay N-DGER)=(assay NASR)=(ACR), was valid. The following discussion examines the validity of the prior art belief that for a prior art particular gene comparison the prior art produced (assay NASR)=(ASR). Because, by definition, the (assay NASR)=(assay N-DGER), the validity of the prior art belief that the (assay N-DGER)=(ACR) will also be examined.

It will be assumed for this discussion that all prior art produced assay NASR values for particular gene comparisons have been produced by: first determining an accurate quantitative measure of the NF value for each prior art known and considered assay variable which is pertinent to the assay; and then normalizing each particular gene comparison assay RAS or RASR value for the prior art NFs which are pertinent to the assay. Such prior art known and considered NFs include the TSAR, ARR, C-HKR, spatial, print tip, print plate, intensity scale, AE•AE, non-specific hybridization, image analysis, background, and random noise NFs (7, 18, 31, 33-35, 41, 51, 88, 128). Many prior art microarray or non-microarray gene comparison assays do not determine assay values for one or more of the prior art known and considered assay variable NF values which are pertinent to the particular gene comparison assay. Such assays produce particular gene comparison assay NASR values which are incompletely normalized with regard to the prior art known and considered assay variables. Similarly, only rarely does prior art RT-PCR practice determine and normalize for the prior art known cDNA AE•SE and cDNA AE•AE assay variables. As an example, many of the microarray assay particular gene comparison assays described in reference (153) are incompletely normalized for prior art considered non-global assay variables.

Does the Prior Art Measured Assay NASR Equal the ASR?

At a given assay ARR value, each of the described assay variables which have been previously utilized for prior art normalization, can influence the measured RASR value for a particular gene comparison. Thus, for a particular gene comparison assay NASR result, if the NF values for the previously known and utilized assay NFs which are pertinent to the assay, accurately reflect the entire set of pertinent assay variables which affect the assay RASR value, then the assay NASR should equal the ACR for the assay. However, if these NFs do not accurately reflect the entire set of pertinent assay variables which affect the assay RASR value, then the assay NASR will not equal the ACR. In this context, it will be useful to identify the known or unknown assay variables which are associated with prior art microarray and other gene expression analysis assays which have not previously been utilized to normalize microarray and non-microarray RASR results, and which may commonly have a significant effect on the assay RASR value for a particular gene comparison. To accomplish this, it will be useful to first discuss the characteristics of the cell sample RNA or mRNA derived labeled polynucleotide molecules, or equivalents which are utilized for microarray and non-microarray gene expression comparison assays. Herein such labeled RNA derived polynucleotide molecules are termed RNA labeled polynucleotide molecules, or RNA LPN molecules, or RNA LPNs. Prior art also utilizes in the microarray and non-microarray assays, LPNs derived from exogenous control polynucleotides which are added to the assay. Herein, these will be termed standard molecule LPNs, or S LPNs. Herein, a polynucleotide molecule directly attached to a signal generation molecule is termed a directly labeled LPN, while a polynucleotide attached to a ligand molecule is termed an indirectly labeled LPN, or indirect LPN.

Characteristics of Gene Expression Analysis Assay Compared LPN Molecules.

Prior art analysis and interpretation of microarray and non-microarray gene comparison results, rely on the assay NASR and N-DGER equaling the ACR value for each particular gene comparison. The NASR for a particular gene comparison, is equal to the normalized ratio of, (the RAS associated with a particular gene in one cell sample)÷(the RAS associated with the same particular gene in a different cell sample). The assay signal itself originates from label molecules, which are associated with the LPN molecules compared in the assay. The signal from a particular label molecule may be fluorescent, or radioactive, or chemiluminescent, or light scattering, electrical or electrical related, or some other.

The LPN molecules used in an assay can be labeled directly or indirectly with a signal generating molecule (7, 8, 13, 151, 152, 154, 155, 156, 157). A directly labeled LPN has one or more label molecules physically attached to the LPN molecule. As a consequence, the label signal molecule is associated with the LPN molecule during the hybridization step, and when a LPN molecule hybridizes, the label signal molecule is carried right along. An indirectly labeled LPN or LPN molecule does not have signal generation molecules directly attached to it, but has one or more ligand molecules attached to it. In some cases, unmodified nucleic acid molecules can act as an indirectly labeled LPN molecules. As an example, an anti-RNA antibody, or an anti-RNA-DNA hybrid antibody attached to a signal label can be used to detect the presence of RNA hybridized to a microarray spot. Indirect label molecules include, but are not limited to Biotin, Avidin, various Haptens, metals, proteins, nucleic acids, glycoproteins, and others. For simplicity, such directly bound entities will be termed ligands.

The indirect label ligand which is directly attached to the LPN, can specifically bind a signal generating label molecule, or a signal generating complex, which contains multiple signal generating molecules. The signal from a particular signal generating molecule or label, may be fluorescent, radioactive, light scattering, chemiluminescent, electrical, or electrically related, or some other. It should be noted that prior art microarray and non-microarray practice assumes that the efficiency of binding the signal generation complex to the ligands associated with the hybridized indirectly labeled LPNs, is the same for all different gene indirect LPNs in an assay.

One or more direct or indirect label molecules may be associated with each LPN molecule. The position of a label in the LPN can vary. One or more particular label molecules may be situated at only the LPN molecules 3′ end, or 5′ end, or at both ends, and nowhere else. This type of LPN is not uncommon in the prior art. Alternatively, multiple labels may be spaced approximately randomly throughout the length of the LPN molecule. This is the most commonly used type of LPN in the prior art. The number of label molecules associated with an LPN molecule varies in different prior art gene comparison assays. Prior art generally attempts to associate as many label molecules as possible with the LPN in order to enhance the assay detection sensitivity. However, too high a label density in the LPN molecules can affect the LPNs ability to hybridize, and can further affect the stability of the hybridized mRNA LPN.

A preparation of directly labeled LPN molecules can be characterized by its quantitative signal activity per mass, usually a microgram, of LPN. Herein, when the LPN signal activity is measured under the signal detection conditions of the assay, this is termed the total LPN signal activity or TSA, for the LPN preparation. For the assay comparison of different directly labeled LPN preparations the ratio of (the TSA for one LPN preparation)÷(the TSA for the other LPN preparation), is termed the TSA ratio or TSAR. Prior art occasionally measures the TSA of fluorescent directly labeled LPNs and often measures the TSA of directly labeled radioactive LPNs (7). Prior art views such differences in the TSA values of different compared LPN preparations as reflecting differences in the efficiencies of labeling and/or label signal detection of each LPN. Prior art generally regards the efficiencies of labeling and signal detection for directly or indirectly labeled LPN preps as global assay variables, which affect all particular gene mRNA LPNs in a cell sample LPN prep in the same manner (7, 50).

It is known that the efficiencies of labeling are often significantly different for different particular gene mRNA LPNs which are present in a cell sample LPN prep. This occurs because different particular gene mRNA transcripts are known to differ in base composition by 3 to 4 fold, and the LPN labeling is often done with a ligand•nucleotide triphosphate precursor which represents only one nucleotide type. It is also known that the efficiencies of labeling are often significantly different for the same particular gene LPN which is present in compared cell sample LPN preps (7, 13, 31, 44, 45, 48, 88, 103, 158, 159, 160). This very often occurs when the same label is used for each compared cell sample LPN, or when a different label is used for each compared cell sample LPN prep. A variety of different cell sample associated factors can cause such differences in the incorporation of the same label in different cell samples. In addition, efficiency of incorporation of different labels into cell sample LPNs is generally significantly different (7). It is also known that the efficiencies of detection of cell sample LPNs which are associated with different labels can be quite different (157, 161, 163). Such differences are due to the intrinsic chemical properties of the different label molecules. As a result of these efficiency of labeling and detection differences the TSA values for compared cell sample LPNs can be significantly different and the assay TSAR value can deviate significantly from one. When the assay TSAR value deviates from one, the assay gene expression analysis results must be normalized for the difference in the TSA values. In the prior art view, since the assay TSAR is a global assay NF, the assay TSAR NF value applies equally to all particular gene expression analysis results in the assay. Such a normalization can be done by dividing each particular gene expression assay result by the assay TSAR value. However, prior art assay TSAR values are rarely used directly to normalize for differences in the labeling efficiency and label signal detection efficiency for the compared LPN preparations. Prior art believes and practices that prior art normalization processes appropriately correct the microarray and non-microarray gene expression analysis results for any differences in the efficiencies of labeling and signal detection for the assay. The validity of this prior art belief and practice depends upon the validity of the prior art assumptions which are necessary in order for the prior art normalization process to be valid. As discussed later, these assumptions are not valid in certain cases, and may not be valid for many others. It should also be noted that the TSAR is not a pure global assay variable NF. The efficiency of labeling of an LPN is influenced by a variety of assay variable factors, some which are global assay variables, and some which can be non-global assay variables. As an example, under certain assay conditions the relative efficiencies of direct or indirect labeling of compared particular LPNs can be affected by differences in nucleotide length, nucleotide sequence and composition, and RNA degradation and purity, which occur within a cell sample mRNA population, and between different cell sample mRNA populations. Similarly, the relative efficiencies of label signal detection of compared particular LPNs can be affected by differences in particular gene LPN label densities which occur within a cell sample mRNA LPN preparation and between the compared cell sample mRNA LPN preparations.

Indirectly labeled cell sample indirect LPN preparations are also employed frequently. The assay TSA value is not applicable to such indirect LPN preparations. For such indirect LPNs, the pertinent labeling parameter is the ligand density. The ligand density for a cell sample indirect LPN prep is the average number of ligands per base in the LPN prep. The relative average ligand densities of compared sample indirect LPN preps can be significantly affected by differences in the compared template RNA nucleotide lengths, nucleotide sequences, nucleotide compositions, degradation, and purity. Some of these factors are associated with global assay variables and others with non-global assay variables.

Indirectly labeled LPN preparations are used for gene expression analysis about as frequently as directly labeled LPN preparations. As discussed, the assay TSA value for a directly labeled LPN prep is influenced by the efficiencies of labeling and label signal detection. The factors which influence the assay TSA value for indirectly labeled LPN preps are more complex, and include, but are not limited to, the following. (i) The number of ligand molecules per average LPN molecule. (ii) The number, or average number of individual signal generating molecules associated with each individual ligand bound signal generation complex molecule or SGC molecule. (iii) The availability of the ligand for binding to the SGC molecule under assay conditions. (iv) The availability of the SGC molecules for binding to the ligand under assay conditions. (v) The efficiency of binding in the assay of available ligands with available SGC molecules. (vi) The stability of the ligand: SGC molecule combination in the assay. (vii) The efficiency of detection of the signal from the ligand bound SGC molecules in the assay. (viii) When an enzyme-substrate reaction is used to generate the assay signal, additional factors, such as substrate availability for the enzyme under assay conditions, enzyme turnover under assay conditions, localization of the substrate product under assay conditions, and others, also influence the assay TSA value for a LPN prep. Many of these factors are non-global assay variables. It is known that the assay values for many of these factors can be significantly different for different prior art compared indirectly labeled LPN preparations, and the SGCs associated with them, and therefore that the assay TSAs for compared indirectly labeled LPN preps can be significantly different. However, prior art compared indirectly labeled assay TSAR values are rarely, if ever, determined and used to normalize prior art gene expression analysis results for differences in the above-described factors which can influence the assay TSAR. Prior art believes and practices that the prior art normalization processes appropriately correct the microarray and non-microarray gene expression analysis assay results for any differences in these factors. The validity of this prior art belief and practice depends upon the validity of the prior art assumptions which are necessary in order for the prior art normalization process to be valid. As discussed later, these assumptions are not valid in certain cases, and may not be valid for many others.

For simplicity in the following discussions the terms LPN and indirect LPN will be designated by LPN. unless otherwise noted. It is not unusual for purified cell sample RNA or isolated cell sample mRNA to be degraded (7, 13, 38, 109, 140, 164-166). Prior art often does not check the degree of degradation of the purified cell sample mRNA before using it in the assay, or before using it to produce mRNA derived LPN molecules. In addition, prior art often does not determine the relative nucleotide lengths of the cell sample LPN molecules which are compared in an assay. The nucleotide length of mRNA LPN molecules can vary with the degree of degradation of the mRNA, the label used, and the purity of the mRNA being labeled. It is further known that mRNA LPN molecules produced from undegraded cell sample mRNA, are almost always significantly shorter in nucleotide length than the undegraded mRNA used to produce the LPN. As a consequence of all this, in a cell sample's mRNA LPN preparation, the nucleotide length or average nucleotide length of a particular mRNA LPN molecule is almost always significantly shorter than the nucleotide length of the undegraded particular mRNA molecule, and may be only a small fraction of the length of the undegraded mRNA or RNA molecule (7, 13, 97, 99, 110, 111, 157, 167-172). As an example, for a mammalian cell sample total mRNA population, the nucleotide length of the average undegraded mRNA transcript molecule is about 2000 nucleotides. For a typical mammalian cell sample cDNA or cRNA prep produced from such undegraded total cell mRNA transcripts, the average nucleotide length of the cDNA or cRNA LPN prep which is produced, generally ranges from an average of about 500-800 nucleotides to an average of 1200-1600 nucleotides (7, 170, 171). Even when producing mammalian cell sample cDNA preps with oligo dT primer the resulting cDNA or cRNA preps have a 500 to 1000 nucleotide average length.

In a cell sample's mRNA LPN preparation, the total nucleotide complexity associated with a particular mRNA's LPN molecules may ideally equal the nucleotide complexity of the undegraded particular mRNA molecule, or may equal only a fraction of the particular undegraded mRNA nucleotide complexity. Herein the total nucleotide complexity is termed the TNC. This can be illustrated by considering a particular undegraded mRNA with a nucleotide length, and nucleotide complexity, of 2000 nucleotides. The TNC of the LPN molecules produced from this intact mRNA may be 2000 nucleotides. This TNC of 2000 can result from two different situations. In one, oligo dT primer is used to produce LPN molecules which are 2000 nucleotides long, and which have a TNC of 2000 nucleotides. Alternatively, the resulting LPN molecules for this particular 2000 nucleotide long mRNA, are produced using random primers. Here, the average nucleotide length in the cell sample mRNA LPN preparation for this particular mRNA's LPN, may be only 500 nucleotides, but since the random primers allowed the entire particular mRNA to be converted to LPN, the aggregate TNC of this particular mRNAs LPN molecules is 2000 nucleotides. In another situation, oligo dT primer is used to produce LPN from this particular undegraded 2000 nucleotide long mRNA, and the maximum nucleotide length of the resulting particular LPN molecules is 700 nucleotides, and the average nucleotide length is about 400 nucleotides. Here the maximum TNC for these particular LPN molecules is 700 nucleotides, and the effective assay TNC is roughly 400-500 nucleotides. That is, the bulk of the particular LPN molecules have a TNC of roughly 400-500 nucleotides. In yet another situation, degraded cell sample total RNA is isolated, and the average nucleotide length of the non-Poly A portion of the particular mRNA molecules is 400 nucleotides, and the maximum length is 700 nucleotides. In the degraded total RNA preparation, the TNC of the particular mRNA molecules is 2000 nucleotides. Here, when the Poly A fraction of the cell sample total RNA is isolated, for the resulting purified Poly A cell sample mRNA the nucleotide length of the average particular mRNA molecules is again about 400 nucleotides with a maximum length of about 700 nucleotides. Here however, the maximum TNC of the particular isolated PA mRNA molecules is not 2000 nucleotides, but 700 nucleotides. In this case the TNC of the particular mRNA LPN molecules produced from this degraded purified cell sample mRNA, using either random or oligo dT primers is about 700 nucleotides. Note that in each of the illustrations where oligo dT primer is used to copy degraded or undegraded mRNA, each cell sample mRNA molecule yields only one LPN molecule per mRNA molecule. For degraded mRNAs, this one LPN molecule represents only the 3′ end of the mRNA molecule. Given that: full sized LPNs for all mRNAs in a cell sample RNA prep is rarely produced, even from undegraded cell sample mRNA; and that it is not unusual for cell sample mRNAs to be degraded and/or differ significantly in purity; and that microarray practitioners seldom determine the nucleotide length of cell sample mRNA and the LPN molecules produced therefrom; it is highly likely that all of the above-described scenarios have occurred and are occurring in prior art microarray practice. The factors which determine the nucleotide length and the TNC of cell sample mRNA LPN molecules include, but are not limited to, the following. The quality of the cell samples used to produce the cell sample RNA. The methods and procedures for isolating and processing cell sample total RNA and mRNA. The purity of isolated total RNA and mRNA. The reagents and procedures used for producing mRNA LPN molecules.

Standard polynucleotide labeling methods can produce two different types of LPN molecules. Herein, these are termed Type 1 and Type 2 LPNs. Both Type 1 (7, 13, 43, 61, 132, 152), and Type 2 (19, 156), LPNs have been used in prior art microarray and non-microarray gene comparison assays, but Type 1 LPNs are by far the most frequently used. Prior art endeavors to compare LPN molecules of the same type in an assay. The two LPN types can be characterized and differentiated by the use of three factors. One factor is the just described total nucleotide complexity or TNC of a mRNA of LPN. A second factor designates for each particular mRNA LPN, the number, or average number, of individual LPN molecules which must be considered in order to determine the TNC for the particular mRNA in the total mRNA LPN preparation. Herein, the number of individual LPN molecules needed to constitute a particular mRNA TNC, is termed the total polynucleotide molecule number, or TPN. The TPN can be illustrated by considering a particular mRNA which is present in cell sample total mRNA, and which has an undegraded nucleotide length and complexity of 2000 nucleotides, and which is used along with an oligo dT primer to produce an LPN preparation from the cell sample mRNA. Here it is assumed that the resulting particular mRNA LPN molecules are full sized, and have a nucleotide length and complexity of 2000 nucleotides. This particular mRNA LPN TNC is 2000 nucleotides. Further, the number of individual LPN molecules which is required to constitute the TNC of 2000 nucleotides, is one. Therefore the TPN=1, for the particular mRNA LPN molecules which are present in the cell sample total mRNA LPN preparation. Note that in the cell sample total mRNA LPN preparation, if all particular short or long mRNA LPN molecules are full sized, then the TPN=1, for all mRNA LPN molecules present in the LPN preparation. For a further illustration, random primers are used to produce an LPN preparation from the cell sample total mRNA containing the particular undegraded mRNA which has a nucleotide length and complexity of 2000 nucleotides. It is assumed that the resulting particular mRNA LPN cDNA molecules which are present in the cell sample total mRNA LPN preparation, are 500 nucleotides in length and have a TNC of 2000 nucleotides. Here a particular mRNA molecule 2000 nucleotides long is represented by, on average, four different particular mRNA LPN molecules, each 500 nucleotides in length on average. Therefore, for this particular mRNA LPN, the TPN=4. In this illustration where the LPN preparation is produced by random priming, the TPN of particular mRNA molecules which have a long undegraded nucleotide length and complexity, can be larger than the TPN of particular mRNA molecules which have a short undegraded nucleotide length and complexity. The nucleotide length and complexity of mammalian cell particular mRNA molecules range from about 200 nucleotides to greater than 6000 nucleotides. Clearly, with random priming the TPN value for different mRNA LPNs present in the cell sample total mRNA LPN preparation, can be very different. A third factor which is useful for characterization and differentiation of Type 1 and Type 2 LPNs, involves the number of label signal or ligand molecules which are associated with each particular LPN molecule which is present in a cell sample mRNA LPN preparation. Herein the number, or average number, of label molecules which are associated with each mRNA LPN molecule, is termed the LPN molecule label number, or LLN. The ratio of the LLN values for a comparison of different cell sample LPN preparations, is termed the LLNR. The LLN can be illustrated by considering a cell sample mRNA LPN preparation produced in a standard manner, by using an oligo dT primer to initiate the incorporation of labeled nucleic acid precursors into the LPN molecules. Here the TPN is equal to one for each of the particular mRNA LPN molecules present in the cell sample mRNA LPN preparation. However, due to the method of labeling, the nucleotide length and the number of label molecules incorporated, will be greater for particular long mRNA derived LPN molecules, than for particular short mRNA derived LPN molecules. Therefore, the LLN is not the same for each LPN molecule present in the cell sample mRNA LPN preparation. For a further illustration, consider a cell sample mRNA LPN preparation produced in a standard manner, by using a random primer to initiate the incorporation of labeled nucleic acid precursors into the LPN molecules. Here the TPN will be greater than one for the bulk of the particular mRNA LPN molecules, and particular LPN molecules from longer mRNAs will have larger TPN values than smaller mRNAs. In addition, because of the method of labeling, the nucleotide length and the number of incorporated label molecules, will be greater for some particular gene mRNA LPN molecules than for others. Thus, the LLN is not the same for each LPN molecule in the cell sample mRNA LPN preparation. As an additional illustration, consider a cell sample mRNA LPN preparation produced in a prior art manner by using oligo dT primer molecules, where each oligo dT primer molecule is associated with or labeled with the same number of label molecules, and no labeled nucleic acid precursor molecules are used. Here the TPN is equal to one for each of the particular mRNA LPN molecules present in the cell sample mRNA LPN preparation. Because of the method of labeling, each LPN molecule in the cell sample mRNA LPN preparation will have the same number of label molecules associated with it. This will be true for both short and long LPN molecules. Therefore, the LLN is the same for each LPN molecule present in the cell sample mRNA LPN preparation. As another illustration, consider a cell sample mRNA LPN preparation produced by using random primers, where each random primer molecule is associated with or labeled with the same number of label molecules, and no labeled nucleic acid precursor molecules are used. Here the TPN will be greater than one for the bulk of the particular mRNA LPN molecules present in the cell sample mRNA preparation, and the LLN will be the same for each LPN molecule present.

All Type 2 cell sample mRNA LPN preparations must have a TPN equal to one or nearly one, for each particular mRNA LPN in the cell sample mRNA LPN preparation, and the same or nearly the same LLN, for each LPN molecule present in the cell sample mRNA LPN preparation.

A Type 1 cell sample mRNA LPN preparation, is one which is not a Type 2 cell sample mRNA LPN preparation. As an example a Type 1 LPN preparation can have a TPN of one for each particular mRNA LPN, and different LLN values for different LPN molecules which are present in the LPN preparation. Alternatively, a Type 1 LPN preparation can have a TPN of two or more for any particular mRNA LPN, and the same LLN value for each LPN molecule present in the LPN preparation. A Type 1 LPN preparation can also have a TPN of one or more for each particular mRNA LPN present in the LPN preparation, and different LLN values for different LPN molecules present in the LPN preparation.

It is useful to measure the label signal activity associated with a Type 2 LPN in terms of label signal activity per LPN molecule. Here, the Type 2 LPN label signal activity of a cell sample LPN prep is termed the LLS. For a cell sample LPN comparison, the LLS value for each compared cell sample LPN may or may not be the same. Here, the ratio of the compared cell sample LPN LLS values is termed the LLS ratio, or LLSR. For a cell sample LPN comparison, even when the LLNR=1, and the same label is used for producing each compared LPN prep, the LLSR may or may not equal one. LLSR values are generally associated with global assay variables, and the LLSR value may or may not equal one.

The above discussion on the characteristics of the cell sample LPN molecules used for gene expression analyzes focused primarily on the SGDS cell sample comparisons of particular gene mRNA transcripts. The discussion also applies directly to LPN molecules produced from standards which are used in the assay. The discussion also applies directly to SGDS, DGDS, and DGSS, assay comparisons of viral, prokaryotic, eukaryotic, and standard RNAs of all kinds. This includes all types of rRNA, tRNA, mRNA, siRNA, miRNA, snoRNA, antisense RNA, and other known and unknown RNAs.

Assay Factors Which Affect the Relationship (NASR)=(ACR).

For a prior art microarray or non-microarray gene mRNA transcript comparison assay, the relationship (NASR)=(ACR) is valid only under certain assay conditions. Certain of these conditions involve prior art known assay variable NFs, which have been previously utilized for normalization of assay gene comparison results. Such NFs include TSAR, C-HKR, spatial, print tip, print plate, intensity, scale, PCR amplification efficiency, background, non-specific hybridization, and image analysis NFs. Other assay factors have been identified, which are associated with assay variables which can significantly affect the assay RASR and which have not been considered for prior art normalization of microarray and/or non-microarray gene expression RASR results. These include the following factors. (i) The nucleotide length or average nucleotide length for the mRNA LPN molecules which are compared in the assay. (ii) The TNC which is present in the assay for each compared particular gene mRNA LPN. (iii) The type, that is Type 1 or Type 2, of LPN molecules compared. (iv) The effective nucleotide length or complexity, of the assay complementary detection polynucleotide used in the microarray assay to detect and quantitate the presence of particular mRNA LPN molecules which are present in the microarray assay hybridization solution. Herein such an assay complementary detection polynucleotide is termed a CDP. The effective CDP or ECDP length or complexity will be discussed below. (v) The quantitative value for each particular mRNA LPN present in the assay for the maximum total nucleotide length of the particular mRNA LPN molecules which can be immobilized or detected in the assay by one CDP molecule. Herein, this is termed the maximum length detectable, or the MLD for a particular mRNA LPN. Herein, the ratio of the compared particular LPN MLD values for a particular gene comparison is equal to the ratio of, (the MLD value for one compared particular mRNA LPN)÷(the MLD value for the other compared particular mRNA LPN), and this ratio is termed the MLDR. The MLD and MLDR will be discussed later. (vi) The effect of the polynucleotide length or average length, of a particular mRNA LPN, on the assay hybridization kinetics of the particular mRNA LPN with its CDP. Herein, the relative hybridization kinetic ratio due to the effect of polynucleotide length on the hybridization kinetics of the compared particular mRNA LPNs, is termed the polynucleotide length hybridization kinetic ratio, or the PL-HKR. Note that different particular mRNA LPN comparisons in one assay can have different PL-HKR values. (vii) The effect of the polynucleotide sequence of a particular mRNA LPN on the assay hybridization kinetics of the particular mRNA LPN with its CDP. Herein, the relative hybridization kinetic ratio due to the effect of the polynucleotide sequence on the hybridization kinetics of the compared particular mRNA LPNs, is termed the polynucleotide sequence hybridization kinetic ratio, or PS-HKR. Note that different particular mRNA LPN comparisons in one assay, can have different PS-HKR values. Note further that the effect of polynucleotide composition on assay hybridization kinetics of particular mRNA LPNs, is included in the PS-HKR value. (viii) The effect of polynucleotide sequence and composition on the label signal activity of a particular gene mRNA LPN. Herein, the signal activity of a particular mRNA LPN which is present in a cell samples mRNA LPN preparation is termed the particular LPN sequence signal activity, or the PSA. Further, the ratio of, (the assay PSA value for a particular gene mRNA LPN from one cell sample)÷(the assay PSA value for the same particular gene's mRNA LPN from a compared cell sample), is termed the PSA ratio, or PSAR. For a particular gene comparison, the assay PSAR value is often not one. The PSA is measured in terms of the signal activity per mass of the particular mRNA LPN. In one cell sample gene comparison assay, different particular gene mRNA LPNs can have different PSAR assay values. Therefore, the PSAR is a non-global NF. (ix) The density of label molecules associated with a particular mRNA LPN can affect, the signal activity associated with the particular mRNA LPN molecules, the hybridization kinetics of the particular mRNA LPN molecule with the CDP, and the assay stability of the resulting CDP hybridized mRNA LPN duplexes. Herein, the label density in an assay of a particular mRNA LPN from one cell sample, is termed the LPN label density, or LD. The LD is measured in terms of the number, or average number, of direct or indirect label molecules per nucleotide base of the LPN molecule. Herein, the ratio of, (the assay LD value for one cell samples particular gene mRNA LPN)÷(the assay LD value for the other compared cell samples same particular gene mRNA LPN), is termed the LD ratio or the LDR. In one cell sample gene comparison assay, different particular gene comparisons can have different assay LDR values. The effect of the assay LDR on a particular gene comparison assay RASR value is complex and will be discussed later. (x) The assay LLSR value for cell sample Type 2 comparisons. (xi) For cell sample indirect LPN comparisons, a measure of the efficiencies of binding of the signal generation complex molecules to a hybridization immobilized indirect LPN molecule, and the stability of the indirect LPN-signal generation complex combination in the assay. A ligand associated signal generation complex molecule is termed an SGC molecule. The number of SGC molecules which can stably bind to a spot immobilized indirect LPN molecule reflects the SGC binding efficiency and the stability of the immobilized indirect LPN SGC complex. Here the number, or average number, of SGC molecules which can stably bind to a hybridization immobilized particular gene indirect LPN molecule, is termed the SGC molecule binding number, or SBN. For a particular gene comparison, the ratio of the compared particular gene SBN values is termed the SBNR. A variety of assay factors can affect the SBNR value. These include but are not limited to the following. (a) The molecular dimensions of the SGC molecules used. (b) The ligand label densities of the compared indirect LPNs. (c) The nucleotide lengths of the compared indirect LPNs. (d) The kinetics of binding of the SGC molecules to the compared immobilized indirect LPNs. (e) The stabilities of the compared immobilized indirect LPN•SGC complexes. Assay factors (b)-(d) can be associated with non-global assay variables, while factor (a) is associated with a global assay variable. Thus, the SBNR can be associated with both global and non-global assay variables. An SGC can bind directly to an indirect LPN molecule, or the binding of the SGC to the indirect LPN can be mediated by another molecule or complex in a sandwich like format. The immobilized ligand can be associated with a double or single strand region of the immobilized indirect LPN molecule. Such a ligand-SGC binding can occur before, after, or during the hybridization step. Prior art practice almost always does the SGC binding to the hybridization immobilized ligand after the post-hybridization wash step, and for simplification this and later discussions will assume this is so, unless otherwise noted. Well known strategies can be used to multiply the number of SGCs associated with an immobilized LPN. Prior art practice often uses such indirectly labeled LPN SGC combinations for microarray assays (156, 173-178). Prior art does not, however, determine SBNR assay values. (xii) For cell sample indirect LPN comparisons the efficiency of signal generation and detection for spot immobilized SGC molecules is measured in terms of the amount of signal activity detected per SGC molecule. Here, the amount of signal activity per immobilized SGC molecule is termed the SGC signal activity or SSA. For a particular gene comparison, the ratio of the compared particular gene SSA values is termed the SSAR. A variety of assay factors can affect the SSAR value for an immobilized SGC molecule. These include, but are not limited to, the following. (a) The type of signal generation molecules compared. (b) The number of signal molecules associated with an SGC molecule. (c) The conditions of signal generation and detection. For a properly designed cell sample indirect LPN comparison, each of these factors should be associated only with global assay variables. (xiii) The linearity of the assay relationship between the assay input of a particular gene RNA versus the observed assay signal associated with the input RNA. The linearity is measured in terms of the slope of the plotted relationship (input RNA amount) versus (observed assay signal). If the slope is one or nearly one for a particular gene RNA, then no normalization is needed for this factor. Microarray assays of most kinds are often associated with slopes which deviate significantly from one. This variable factor can be global or non-global in nature. (xiv) The amount of second strand cDNA synthesis which occurs during the first strand reverse transcriptase synthesis step for a particular RNA. This variable can be global or non-global in nature, but is likely to be non-global.

Note that all of the above-noted unconsidered assay variables are associated with non-global assay variables except the LLSR and possibly the SSAR. Most, if not all, of these assay variables can cause an assay measured particular gene RASR to deviate from the assay ACR and biological accuracy by 1.5 to 2 fold or more. In aggregate, the product of these unconsidered assay variable effects have the potential to cause an assay measured particular gene RASR value to deviate from the assay ACR value and biological accuracy by 10 to 20 fold or more. Each of these unconsidered assay variables is discussed below. This discussion includes the effect of a prior art considered assay variable associated NF, the PCR associated AE•AE NF or the PCR amplification efficiency. This considered NF is included here as it affects the validity of the RT-PCR assay relationship (NASR)=(ACR), and the prior art determination and normalization for this factor is not valid.

The following discussions on the validity of the relationship (NASR)=(ACR), applies directly to all SGDS, DGDS, and DGSS comparisons of viral, prokaryotic, eukaryotic, and standard RNAs of all kinds. This includes all types of rRNA, tRNA, mRNA, siRNA, miRNA, snoRNA, antisense RNA, and other known and unknown RNAs. TSAR and PSAR of LPNs.

For a cell sample gene expression analysis comparison the ratio of (the TSA for one cell sample's mRNA LPN preparation)÷(the TSA for a different cell sample's mRNA LPN preparation), is termed the TSA ratio, or TSAR. The TSA of an LPN preparation is measured in terms of the quantity of label signal activity per microgram of LPN as measured under the assay signal activity detection conditions. The TSA value for a cell sample mRNA LPN preparation is a measure of the signal activity per microgram of the total cell sample mRNA LPN preparation. However, a particular gene mRNA LPN molecule population present in the total cell sample mRNA LPN preparation, can have a significantly different label signal activity value per microgram of the particular gene mRNA LPN, when measured under assay conditions. Herein, the particular gene signal activity per microgram of particular gene mRNA LPN is termed the particular gene signal activity, or the PSA, and the ratio of (the PSA for a particular gene mRNA LPN which is present in one compared cell sample mRNA LPN prep)÷(the PSA for the same particular gene mRNA LPN which is present in a different compared cell sample mRNA LPN prep), is termed the PSA ratio or PSAR. The PSA value for a particular gene mRNA LPN in a cell sample mRNA LPN prep reflects the efficiencies of labeling and signal activity detection for the particular gene mRNA LPN. Thus, the assay PSAR value for a cell sample LPN prep comparison reflects the relative efficiencies of labeling and signal activity detection for the particular gene mRNA LPNs.

It is known that the PSA values of different particular gene mRNA LPNs in one cell sample mRNA LPN prep can be significantly different. As an example, particular gene mRNAs present in a cell sample total mRNA preparation have significantly different nucleotide sequences and nucleotide compositions. By far the most commonly used method for producing mRNA LPNs utilizes a DNA or RNA polymerase to incorporate deoxy or ribo labeled ATP or UTP, or CTP into mRNA LPN cDNA or cRNA molecules. In such a situation, particular gene mRNA LPNs produced from mRNAs which have a high adenine, guanine, or uridine content, will contain more label per microgram of mRNA LPN, than those particular gene LPNs produced from mRNAs which have a relatively low adenine or guanine content. Such contents can vary by about 3-4 fold for different particular gene mRNAs and for particular nucleotide sequences in one particular RNA.

It is also known that the PSA value for a particular gene mRNA LPN which is present in one cell sample mRNA LPN prep can be significantly different from the PSA value for the mRNA LPN of the same particular gene which is present in a different, compared cell sample mRNA LPN prep. These PSA differences can be caused by differences in labeling efficiency and/or label signal activity detection efficiency between the compared LPNs and particular gene mRNA LPNs, which are associated with differences in the nucleotide length, nucleotide sequence, nucleotide composition, RNA purity, LPN labeling density, and other factors, which can exist for the compared cell sample RNAs and/or LPN equivalents and/or the compared particular gene RNAs and/or equivalent LPNs. Such differences are not uncommon for prior art gene expression analysis microarray and non-microarray assays. Further, such differences can cause compared particular gene mRNA PSA values to differ by 2-4 fold or more. Note that many of these differences which can result in different PSA values are associated with non-global assay variables, and non-global assay variable NFs.

For a cell sample mRNA LPN comparison, the TSAR and PSAR are assay variable NFs. Prior art believes that the TSAR is a global assay variable NF. However, prior art seldom, if ever, directly determines the assay TSAR and uses it directly to normalize gene expression analysis results. As discussed, for a cell sample mRNA LPN comparison assay, the PSAR NF values for particular gene mRNA LPN comparisons can be significantly different from the TSAR NF value. Because of this the assay TSAR NF value may not correctly or completely normalize particular gene mRNA LPN comparison assay results for differences in the efficiency of labeling and/or signal detection of compared particular gene mRNA LPNs. Further, absent some knowledge of the assay PSAR values for particular gene mRNA LPN comparisons, it cannot be known whether the TSAR correctly and completely normalizes the particular gene comparison results or not. Prior art microarray and non-microarray gene expression analysis practice neither determines nor considers the PSAR NF values for particular gene mRNA LPN comparisons. In this context, it cannot be known whether prior art normalized particular gene mRNA LPN comparison results, are completely and correctly normalized or not.

Most prior art cell sample mRNA LPN preparations are produced by chemically or enzymatically incorporating label molecules more or less randomly along the length of the LPN molecule. For such an LPN molecule, the number of associated label molecules almost always increases in direct proportion to LPN molecule nucleotide length (7). Here, a particular gene mRNA LPN molecule population which consists of long nucleotide sequence molecules is generally associated with more label molecules per LPN molecule, than is a shorter LPN molecule from a different particular gene mRNA LPN population which consists of shorter LPN molecules. Similarly, for a cell sample particular gene mRNA LPN comparison which has an assay PSAR=1, when one cell samples particular gene mRNA LPN consists of long LPN molecules, and the other cell samples same particular gene mRNA LPN consists of short LPN molecules, then the signal activity per LPN molecule is greater for each longer LPN molecule than for each short LPN molecule. Consequently, the signal activity obtained from one long LPN hybridized to a spot immobilized CDP, will be greater than the signal activity obtained when one short LPN molecule hybridizes to the same spot immobilized CDP. Such differences in nucleotide length between compared particular gene mRNA LPNs from different cell samples, are not uncommon. Prior art generally does not determine and/or report the relative nucleotide lengths of compared cell sample mRNA LPN preps, and further does not determine and/or report the relative nucleotide lengths of compared particular gene mRNA LPNs.

Note that each DGDS and DGSS particular gene comparison is also associated with a PSAR value. The above discussion also applies to these particular gene comparisons.

CDP and Effective CDP Complexity.

Spot immobilized polynucleotide which is complementary to a particular mRNA LPN, is used in a microarray assay to detect and quantitate the presence of the particular mRNA LPN molecules in the assay hybridization solution (7, 58, 84, 179-185). Such spot immobilized polynucleotides are often termed capture probes. A CDP consists of a single or double stranded DNA or RNA polynucleotide, which can be as short as 15-20 and as long as thousands of nucleotides in length. The lower limit of 15-20 nucleotides represents the shortest complementary polynucleotide molecule, which can reliably be used to specifically detect an LPN molecule in an assay. Prior art microarray individual CDPs generally range in nucleotide length and complexity, from about 20 to 1200 nucleotides, but can be much longer. Oligonucleotide microarray CDP nucleotide length and complexity ranges from about 20-80 nucleotides, while most cDNA microarray CDPs are around 400-1200 nucleotides in length. Each CDP molecule is immobilized on the array surface in a single strand state. Each particular gene CDP is sited in a separate physical location on the surface of the array or assay device. Generally each separate spot contains only one CDP type, which has a single nucleotide complexity and length, as well as its own effective CDP nucleotide complexity and length. A particular CDP molecule type may contain one or more nucleotide sequences which are complementary to a particular mRNA or control LPN molecule population, and one or more nucleotide sequence regions which are not complementary to any particular cell sample or control mRNA or polynucleotide LPN present in the assay. Microarray CDP molecules are often designed to represent the 3′ portion of a particular gene mRNA molecule.

The effective CDP complexity or nucleotide length, is equal to the nucleotide complexity or length of a particular CDP molecule, which is complementary to and can hybridize with, the particular mRNA LPN molecules which the CDP is designed to detect in the assay hybridization solution. Prior art practice for microarray assay SGDS gene comparisons is to use only one CDP per spot for a particular cell sample mRNA. The effective CDP complexity or length in the assay can be equal to or less than, the nucleotide length or complexity of the particular mRNA the CDP is complementary to. Further, the effective CDP complexity or length in the assay, can be greater than, equal to, or less than, the nucleotide length of the LPN molecules which hybridize to it in the assay. Typically the effective CDP nucleotide length and complexity is significantly shorter than its full length mRNA. Herein, the effective CDP complexity and length is termed the ECDP.

The ECDP can be illustrated by considering a situation where, the particular gene mRNA LPN molecules present in the assay hybridization solution have a total nucleotide complexity (TNC) of 1000 nucleotides, and the spot immobilized CDP molecule contains a nucleotide sequence which has a total nucleotide complexity and length of 300 nucleotides which is complementary to the particular mRNA LPN molecules of interest. In this situation, the ECDP is equal to 300 nucleotides. Here the particular ECDP composition could consist of: 5 different 60 nucleotide long polynucleotide molecules, each with a different nucleotide sequence; or a single 300 nucleotide long molecule; or one or more complementary nucleotide sequences, interspersed among nucleotide sequences which are not complementary to the particular mRNA LPN molecules of interest.

As a further illustration, consider a situation where the particular mRNA molecule of interest has an undegraded nucleotide complexity and length of 2000 nucleotides. However, due to degradation in the cell sample mRNA LPN preparation, the TNC for the particular mRNA molecule population is only 500 nucleotides. The entire 1000 nucleotide length of the immobilized CDP for this particular mRNA LPN is perfectly complementary to the undegraded particular mRNA molecule. Here, since only 500 nucleotides of the CDP are complementary to the particular mRNA LPN molecules which are present in the assay hybridization solution, then the ECDP is equal to 500 nucleotides.

As an additional illustration, consider a situation where the particular mRNA LPN molecules of interest have a TNC of 2000 nucleotides, and an average nucleotide length of 400 nucleotides. Further, the assay CDP for this particular mRNA LPN molecule population, consists of an immobilized 20 nucleotide long oligonucleotide, which is completely complementary to the particular mRNA LPN of interest. Here the ECDP is 20 nucleotides.

In many prior art microarray gene comparison assays, different particular mRNA LPNs are often associated with different ECDP values, and when degradation of cell sample mRNA occurs, a particular mRNA's ECDP value for one cell sample can be significantly different from the ECDP associated with the same mRNA in a different cell sample.

Note that a prior art discussion of either the ECDP or the use of the ECDP for the normalization of gene expression assay results has not been discovered.

In contrast to the microarray CDP molecule which is unlabeled, a CDP for the non-microarray gene expression assay methods northern blot, dot blot, and nuclease protection, consists of a labeled polynucleotide which is complementary to the unlabeled particular mRNA of interest. In addition, for nuclease protection the CDP is not immobilized. RT-PCR assays generally do not have CDP molecules.

The MLD and MLDR Assay Factors.

The assay values for three of the earlier described assay factors, are required in order to derive the assay MLD value for each particular mRNA LPN of interest. The three factors are: (i) The nucleotide length or average nucleotide length in the assay, of the particular mRNA LPN molecules of interest; (ii) The TNC of the particular mRNA LPN of interest; (iii) The assay ECDP for the particular mRNA LPN of interest.

The use of these three assay factors to determine an assay MLD value for a particular gene comparison is illustrated in Table 14. Scenario A considers the following situation. (i) The nucleotide length and complexity of the undegraded mRNA of interest is 2000 nucleotides. (ii) The TNC of the mRNA LPN present in the assay is also 2000 nucleotides. The nucleotide length of the mRNA LPN molecules is also 2000 nucleotides. (iii) The ECDP of the mRNA LPN of interest is 20, 200, or 2000 nucleotides. Here, for short and long ECDP values the assay MLD is the same, 2000 nucleotides, since any stable hybridization event between a single short or long CDP molecule and a 2000 nucleotide long mRNA LPN molecule, will always result in the entire 2000 nucleotide long mRNA LPN molecule being immobilized in the CDP spot. Here then, the maximum mRNA LPN length which can be detected by one CDP molecule is 2000 nucleotides.

TABLE 14
Determination of the Microarray Assay MLD Value for
Particular mRNA LPN Molecules from One Cell Sample

| | | | | | | (MLD) |
|---|---|---|---|---|---|---|
| | | | | Nucleotide | | Maximum |
| | | Nucleotide | | Length of | | Length of |
| | | Complexity | TNC of | mRNA 1 | ECDP for | mRNA 1 LPN |
| Cell | | of | mRNA 1 | LPN | mRNA 1 | Molecules |
| Sample | Assay | Undegraded | LPN in | Molecules in | LPN in | Which is |
| Gene | Scenario | mRNA 1 | Assay | Assay | Assay | Detectable |
| 1 | A | 2000 | 2000 | 2000 | 20 | 2000 |
| | A | 2000 | 2000 | 2000 | 100 | 2000 |
| | A | 2000 | 2000 | 2000 | 2000 | 2000 |
| 1 | B | 2000 | 2000 | 100^{(a)} | 2000 | 2000 |
| 1 | C | 2000 | 2000 | 100^{(a)} | 30 | 100 |
| 1 | D | 2000 | 1000^{(a)} | 1000^{(a)} | 80 | 1000 |
| 1 | E | 2000 | 200^{(a)} | 200^{(a)} | 80 | 200 |
| 1 | F | 2000 | 1000^{(a)} | 1000^{(a)} | 80 | 1000 |
| 1 | G | 2000 | 2000 | 200^{(a)} | 1000 | 1000 |
| 1 | H | 2000 | 400^{(a)} | 200^{(a)} | 1000 | 400 |

^{(a)}Less than complete nucleotide complexity, or length may be due to RNA degradation or the labeling process, or both.

Scenario B considers a situation identical to that of Scenario A, except that the nucleotide length or average nucleotide length of the mRNA LPN molecules present in the assay is 100 nucleotides, and the assay ECDP value is 2000 nucleotides. In this situation the mRNA TNC is 2000 nucleotides, and because the nucleotide length of the mRNA LPN molecules present in the assay is only 100 nucleotides, the TNC of 2000 nucleotides represents 20 different 100 nucleotide long mRNA LPN molecules. That is the particular mRNA LPN TPN value is equal to 20. Since the ECDP is 2000 nucleotides, each different 100 nucleotide long LPN molecule can separately hybridize to a single CDP molecule. Here then, the maximum mRNA LPN length which can be immobilized or detected by one CDP molecule is 2000 nucleotides, and therefore the MLD equals 2000 nucleotides, even though the nucleotide length of the mRNA LPN molecules is only 100 nucleotides.

Scenario C considers a situation, which is identical to that of Scenario B, except that the mRNA LPN ECDP is equal to 30 nucleotides. In this situation only one of the 20 different 100 nucleotide long mRNA LPN molecules which represent the TNC of 2000 nucleotides, can hybridize to a single 30 nucleotide long CDP molecule. Here then, the maximum mRNA LPN length which can be immobilized or detected by one CDP molecule is 100 nucleotides, and therefore the assay MLD equals 100 nucleotides.

Scenario D considers a situation where: The undegraded nucleotide length of the mRNA of interest is 2000 nucleotides; and the TNC of the mRNA LPN present in the assay is 1000 nucleotides due to mRNA degradation; and the nucleotide length of the mRNA LPN molecules present in the assay is 1000 nucleotides; while the assay ECDP is 80 nucleotides. In this situation the mRNA LPN TNC is represented by one 1000 nucleotide long mRNA LPN molecule, that is the TPN of the mRNA LPN is equal to 1. In the assay only one 1000 nucleotide long LPN molecule can hybridize to a single 80 nucleotide long CDP molecule. Here then, the MLD is equal to 1000 nucleotides.

In the light of the above-described illustrations, Scenarios E-H are self-explanatory. Further, the above examples are idealized for simplicity, and these idealized aspects will be recognized and taken into consideration by one of skill in the art. These illustrations provide a basis for determining the assay MLD value for any particular mRNA LPN, in any assay for which the proper information can be determined.

Table 14 indicates that a particular mRNA's assay MLD may be widely different for different mRNA LPN preparations from one cell sample, depending on the quality of the cell sample mRNA, and the efficiency and details of the LPN production. Table 15 illustrates that different particular mRNA LPNs in one cell sample LPN mRNA preparation, can have different assay MLD values.

For an SGDS microarray gene comparison assay the ratio of, (the assay MLD value of a particular mRNA LPN from one cell sample)÷(the assay MLD value of the sample particular mRNA LPN from a different cell sample), is termed the MLD ratio, or MLDR.

TABLE 15
Determination of the Microarray Assay MLD Value for Different Particular
mRNA LPN Molecules in One Cell Sample mRNA LPN Preparation

| | | Nucleotide | TNC of | Nucleotide | ECDP | MLD | |
|---|---|---|---|---|---|---|---|
| | | Complexity | mRNA | Length of | for | for | |
| Cell Sample | Cell | of | LPN | mRNA | mRNA | mRNA | LPN |
| mRNA LPN | Sample | Undegraded | Molecules | LPN in | LPN in | LPN in | Labeling |
| Preparation | Gene | mRNA | in Assay | Assay | Assay | Assay | Method |
| I | 1 | 2000 | 2000 | 2000 | 700 | 2000 | Oligo dT |
| | 2 | 1000 | 1000 | 1000 | 500 | 1000 | Primer |
| | 3 | 500 | 500 | 500 | 300 | 500 | (assumes |
| | 4 | 200 | 200 | 200 | 200 | 200 | that full |
| | | | | | | | sized LPN |
| | | | | | | | molecules |
| | | | | | | | are |
| | | | | | | | produced) |
| II | 1 | 2000 | 2000 | 400 | 900 | ˜1200 | Random |
| | 2 | 2000 | 2000 | 400 | 300 | ˜400 | Primer |
| | 3 | 2000 | 2000 | 400 | 700 | ˜800 | |
| | 4 | 1000 | 1000 | 400 | 300 | ˜400 | |
| | 5 | 200 | ˜150 | ˜150 | 150 | ˜150 | |

Table 16 presents the determination of the assay MLDR values for the comparison of Gene B mRNA LPNs produced from Cell Sample 1 and Cell Sample 2. Clearly the MLDR value for the Gene B mRNA LPN comparison can vary widely, depending on the relative differences in nucleotide length, the TNC of the compared Gene B mRNA LPN molecules, and the ECDP of the Gene B CDP. Table 17 presents the determination of assay MLDR values, for different SGDS gene comparisons, which occur in one assay comparison. Here the MLDR values for different gene comparisons in the same assay are not necessarily the same, depending on the nucleotide length and TNC of the compared mRNA LPNs, and the mRNA LPNs assay ECDP value.

TABLE 16
Determination of Assay MLDR Values for A Particular mRNA LPN SGDS
Gene Comparison

| | | | | | | | MLD | |
|---|---|---|---|---|---|---|---|---|
| | | | | | | ECDP | Value | |
| | | | | TNC in | Nucleotide | for | for | Gene |
| | | | Undegraded | Assay | Length of | mRNA | mRNA | Comparison |
| | | Compared | mRNA B | of | LPN | B LPN | B LPN | (B1/B2) |
| Compared | | Cell | Nucleotide | mRNA | Molecules | in | in | MLDR in |
| Gene | | Sample | Complexity | B LPN | in Assay | Assay | Assay | Assay |
| (i) | | 1 | 2000 | 2000 | 2000 | 50 | 2000 | 1 |
| | B | 2 | 2000 | 2000 | 2000 | 50 | 2000 | |
| (ii) | | 1 | 2000 | 2000 | 200^{(a)} | 50 | 200 | 1 |
| | B | 2 | 2000 | 2000 | 200^{(a)} | 50 | 200 | |
| (iii) | | 1 | 2000 | 2000 | 2000 | 50 | 2000 | 10 |
| | B | 2 | 2000 | 200^{(a)} | 200^{(a)} | 50 | 200 | |
| (iv) | | 1 | 2000 | 1000^{(a)} | 200^{(a)} | 1000 | 1000 | 2.5 |
| | B | 2 | 2000 | 400^{(a)} | 200^{(a)} | 1000 | 400 | |
| (v) | | 1 | 2000 | 500^{(a)} | 500^{(a)} | 400 | 500 | 0.25 |
| | B | 2 | 2000 | 2000 | 2000 | 400 | 2000 | |
| (vi) | | 1 | 2000 | 2000 | 2000 | 500 | 2000 | 20 |
| | B | 2 | 2000 | 100^{(a)} | 100^{(a)} | 100^{(b)} | 100 | |
| (vii) | | 1 | 2000 | 1200 | 1200 | 300 | 1200 | 3 |
| | B | 2 | 2000 | 400 | 400 | 300 | 400 | |

^{(a)}Less than complete LPN complexity or length may be due to RNA degradation or the labeling procedure or both.
^{(b)}While only one CDP spot is used for both B1 and B2 in a SGDS gene comparison assay, it is possible to have two different ECDP values for the same CDP.

TABLE 17
Determination of Microarray Assay MLDR Values for Different Particular
mRNA LPN Gene Comparisons in the Same Assay

| | | | | | | | Gene |
|---|---|---|---|---|---|---|---|
| | | Nucleotide | TNC of | Nucleotide | ECDP | MLD | Comparison |
| | | Complexity | mRNA | Length of | for | for | (Sample |
| | Compared | of | LPN | mRNA | mRNA | mRNA | 1/Sample 2) |
| Compared | Cell | Undegraded | Molecules | LPN in | LPN in | LPN in | MLDR in |
| Gene | Sample | mRNA | in Assay | Assay | Assay | Assay | Assay |
| Assay I | | | | | | | |
| A | 1 | 2000 | 2000 | 2000 | 500 | 2000 | 1 |
| A | 2 | 2000 | 2000 | 2000 | 500 | 2000 | |
| B | 1 | 1000 | 1000 | 1000 | 400 | 1000 | 1 |
| B | 2 | 1000 | 1000 | 1000 | 400 | 1000 | |
| C | 1 | 400 | 400 | 400 | 200 | 400 | 1 |
| C | 2 | 400 | 400 | 400 | 200 | 400 | |
| Assay II | | | | | | | |
| A | 1 | 2000 | 2000 | 2000 | 200 | 2000 | 5 |
| A | 2 | 2000 | 400^{(a)} | 400^{(a)} | 200 | 400 | |
| B | 1 | 1000 | 1000 | 1000 | 300 | 1000 | 2.5 |
| B | 2 | 1000 | 400^{(a)} | 400^{(a)} | 300 | 400 | |
| C | 1 | 300 | 300^{(a)} | 300^{(a)} | 150 | 300 | 1 |
| C | 2 | 300 | 300^{(a)} | 300^{(a)} | 150 | 300 | |
| Assay III | | | | | | | |
| A | 1 | 2000 | 2000 | 400^{(a)} | 200 | 400 | 1 |
| A | 2 | 2000 | 400^{(a)} | ˜400^{(a)} | 200 | 400 | |
| B | 1 | 2000 | 2000 | 400^{(a)} | 1000 | 1000 | 2.5 |
| B | 2 | 2000 | 400^{(a)} | ˜400^{(a)} | 1000 | 400 | |

^{(a)}See footnote (a) of Table 16.

For an SGDS gene comparison analysis where: Type 1 LPN molecules are compared; the compared cell sample mRNAs are always undegraded; the mRNA labeling process always works perfectly, thereby producing full sized mRNA LPN molecules for all particular mRNAs in a cell sample mRNA preparation; the MLDR for any particular mRNA LPN SGDS gene comparison would always equal one, and could be ignored as an assay variable, as the prior art does. Note that for DGDS or DGSS comparisons this may not be true. Table 17 Assay I illustrates this for an SGDS comparison assay. In order to obtain these ideal results it is necessary to produce full length mRNA LPN using oligo dT primers from undegraded cell sample mRNA, or by chemically labeling undegraded cell sample mRNA without degrading it. This rarely, if ever occurs in reality.

In reality, it is not uncommon for isolated cell sample RNA to be degraded to a greater or lesser extent. In addition, it is known that different cell sample preparations of RNA often vary in purity. It is also known that mRNA LPN molecules produced from undegraded mRNA are generally significantly shorter in nucleotide length than the undegraded mRNA molecules used to produce the mRNA LPN, and that factors related to the isolation, purification, and processing, of RNA can have a great effect on the nucleotide length of LPN molecules and the TNC for a particular RNA LPN. These by no means rare imperfections, impact the production of reproducible cell sample RNA LPN molecules, and indicate that it is not reasonable to believe that the microarray assay SGDS MLDR value for each particular gene comparison in an assay is equal to one, and can therefore be ignored during the normalization process. Tables 16 and 17 illustrate the effect of the assay ECDP value, and differences in the nucleotide length, and the TNC of compared particular mRNA LPNs, on the assay MLDR for those gene comparisons. Consider the example in Table 16 (iii). Here, Cell Sample 1 mRNA B is undegraded and produces full sized 2000 nucleotide long LPN molecules, which also have a TNC of 2000 nucleotides. In contrast, the compared Cell Sample 2 mRNA is seriously degraded, and the oligo dT primer label method produces mRNA B LPN molecules which have a TNC of 200 nucleotides and a nucleotide length of 200 nucleotides. A single 50 nucleotide long ECDP molecule, can hybridize to only one 2000 nucleotide long LPN molecule from Cell Sample 1, or one 200 nucleotide long LPN molecule from Cell Sample 2. Here, the (Cell Sample 1 MLD)÷(Cell Sample 2 MLD) ratio, or MLDR is equal to 10. Such a situation arises because one of the compared cell sample mRNA's is seriously degraded, and the mRNA LPN was produced using oligo dT primers. Other examples which are consistent with using oligo dT primers to produce LPN molecules are Table 16 (i) (v) (vi) (vii), and Table 17 Assay I, and Assay II. Consider also the example of Table 16 (iv). This example is consistent with a situation where the Cell Sample 1 mRNA was mildly degraded, and the Cell Sample 2 mRNA was seriously degraded, before the Poly A mRNA from each cell sample was isolated. As a result, the purified Cell Sample 1 and Cell Sample 2 purified mRNA nucleotide lengths were respectively, 1000 nucleotides and 400 nucleotides. Random primers were then used to make the respective mRNA LPNs, and the nucleotide length of each of these LPN molecule populations is 200 nucleotides. Here, a single 1000 nucleotide long ECDP molecule, can hybridize to five of the 200 nucleotide long Cell Sample 1 mRNA B LPN molecules, and to only two of the 200 nucleotide long Cell Sample 2 mRNA B LPN molecules. The assay MLDR is then equal to 2.5. Other examples, which utilize the random primer method of labeling LPN molecules, are Table 16 (ii), and Table 17 Assay III. Tables 16 and 17 illustrate that differences in the nucleotide length, and TNC of compared particular mRNA LPN molecules, can cause the resulting assay MLDR values for particular SGDS mRNA LPN comparisons to deviate significantly from one.

Note that an MLDR value is also associated with each DGDS and DGSS particular gene comparison.

The Assay Factor PL-HKR.

For a SGDS microarray or non-microarray particular gene mRNA LPN comparison, when the compared LPN molecules have the same nucleotide length and nucleotide sequence, there will be no nucleotide length or nucleotide sequence dependent differences in the hybridization kinetics of each compared LPN molecule population with the CDP. However, it is known that the kinetics of LPN hybridization with its immobilized CDP is affected by the nucleotide length of the LPN (186, 187). For hybridization reactions where both complementary strands are free in solution, the hybridization rate is faster for longer LPN molecules than for short LPN molecules. In solution, the rate increases as the square root of the proportional increase in nucleotide length, and a 10 fold difference in length will result in the longer LPN hybridizing about three times faster than the short LPN. In contrast, the hybridization of short LPNs with a spot immobilized CDP will be faster than that of long LPNs. It has been reported that the hybridization kinetics of long and short LPNs with an immobilized CDP differ by about the square root of the length difference between them (186). This indicates that a 200 nucleotide long LPN will hybridize about two times faster than an 800 nucleotide long LPN.

For a gene comparison of the same particular LPN molecules from different cell samples, the effect of differences in nucleotide length between the two compared LPNs on the assay LPN hybridization kinetics can be described by the relative difference in the hybridization kinetics of the compared LPNs with the genes CDP. Herein, this relative difference is described as the polynucleotide length hybridization kinetic ratio, or the PL-HKR, for a particular gene comparison. It seems plausible that assay PL-HKR values, which deviate from one by two fold or so, are not uncommon for prior art assays. Note that the PL-HKR can be used to normalize gene comparison results for the polynucleotide length effect on the assay hybridization kinetics, and that the PL-HKR may be different for different particular gene comparisons in an assay. PL-HKR is a non-global NF. Prior art seldom determines the nucleotide lengths of the compared cell sample LPN molecules, and does not take the PL-HKR into consideration during the process of normalizing gene comparison assay results.

Note that a PL-HKR value is also associated with each DGDS and DGSS particular gene comparison.

The Assay Factor PS-HKR.

For an SGDS microarray or non-microarray particular gene mRNA LPN comparison, when the compared LPN molecules each have the same nucleotide length and nucleotide sequence, there will be no nucleotide sequence or nucleotide composition related difference in the hybridization kinetics of each LPN with the particular gene CDP. Here, the PL-HKR=1, and the PS-HKR=1, for the particular gene LPN comparison. However, when the nucleotide length or TNC of one compared particular gene LPN, differs from the other compared LPN, the nucleotide sequence of the longer compared LPN is different, at least in part, from the nucleotide sequence of the other shorter compared LPN. Because of the different nucleotide sequence the nucleotide composition of the longer compared LPN may be significantly different than the nucleotide composition of the shorter compared LPN. The effect of this nucleotide composition difference on the assay PL-HKR value for this particular gene LPN comparison will depend on the magnitude of the nucleotide composition difference. For such a particular gene LPN comparison the PL-HKR may or may not equal one or nearly one, depending on the magnitude of the nucleotide length difference, and the PS-HKR may or may not equal one or nearly one, depending on the magnitude of the nucleotide sequence and/or composition difference. Assay factors related to the isolation, purification, and processing cell sample mRNA, and to the production of mRNA LPN molecules, can have a great effect on the nucleotide length and TNC for a particular mRNA LPN in a cell sample's total mRNA LPN preparation. Because of these factors, it is reasonable to believe that for many prior art particular gene comparisons the nucleotide lengths and/or the TNCs of the compared mRNA LPNs are different, and therefore the polynucleotide sequences and/or compositions of the compared particular gene mRNA LPN molecules are not the same. This raises the possibility that the compared LPNs may differ significantly in nucleotide sequence and composition, and that the PS-HKR≠1 for the LPN comparison.

It is known that when the DNA molecule nucleotide complexity and nucleotide length and nucleotide composition are controlled for, then differences in nucleotide sequence have little effect on the basic kinetics of hybridization of DNA molecules of moderate length which are free in solution. However, when the DNA molecule nucleotide complexity and nucleotide length are controlled for, differences in nucleotide composition can affect the in solution hybridization kinetics, and high (64%) G+C DNA hybridizes about twofold faster than low, 34%, G+C DNA (187). Note that the G+C content of different mammalian mRNAs range from about 25-75%, and the G+C content of different regions of the same mRNA can differ very significantly. The effect of G+C content differences on the hybridization kinetics of compared LPNs to a gene CDP, is not known. It is likely, however, that there is some effect, but whether it is larger or smaller than the in solution effect is not known. Such information can be determined by experimentation.

Another nucleotide sequence related factor which can influence the hybridization kinetics and PS-HKR of compared particular gene LPN molecules, involves the nucleotide sequence related secondary structure of compared LPN molecules. It is known that strong nucleotide sequence dependent secondary structure in a nucleic acid single strand molecule, can greatly slow or even prevent the hybridization of a short nucleic acid molecule with a complementary short or long nucleic acid molecule. In general the shorter the nucleotide length of the nucleic acid containing the strong secondary structure, the greater the potential for reducing the hybridization kinetics. Similarly, the existence of a sequence dependent region of strong secondary structure in a long nucleic acid, can greatly slow the rate of hybridization of a short complementary nucleic acid with the strong secondary structure region of the long nucleic acid molecule. Again, the shorter the nucleic acid molecule which is trying to hybridize to the region of strong secondary structure on the long molecule, the greater the potential for slowing the hybridization rate. Here, the longer the short nucleic acid molecule is, the less the effect of the region of strong secondary structure in the long molecule has on the basic hybridization rate between the short and long molecules. When the short molecule gets long enough, the strong secondary structure region has little effect on the hybridization rate for the short and long molecules. When the short molecules have a nucleotide length in the range of very roughly 100-300 nucleotides, such sequence effects appear to be minimal for the vast majority of different sequences. Because of this, sequence secondary structure related hybridization kinetic differential inhibition effects in cDNA microarrays should be minimal and the probability of any one particular gene LPN comparison being affected is low. For cDNA microarrays the gene ECDP nucleotide length is almost always greater than 100 nucleotides long, and generally ranges from 200-1200 nucleotides long. In addition, the nucleotide length or TNC for any particular gene LPN is virtually always greater than 80 nucleotides long. In contrast, for oligonucleotide arrays the probability of any one particular gene LPN comparison being associated with such secondary structure related differential hybridization effects is much higher than for the cDNA microarray assays, and such effects may be a serious problem for many oligonucleotide array based assays. For oligonucleotide microarrays in general, the ECDP nucleotide length for any particular gene ranges from about 20 to 80 nucleotides, and for a particular oligonucleotide microarray, the ECDP nucleotide length for all genes is generally about the same. As an example Affymetrix oligonucleotide microarray's ECDP for all genes is generally around 25 nucleotides, and for the GE-Amersham codelink oligonucleotide microarrays, the ECDP for all genes is about 30 nucleotides, while for the Agilent oligonucleotide microarrays the ECDP for all genes is about 60 nucleotides. Prior art practice is to select oligonucleotide molecules which are capable of giving “strong” signals when hybridized to mRNA LPN molecules in an assay. However, it is not evident that all of the oligonucleotide molecules selected for inclusion on the microarray have the same, or nearly the same, basic rate of hybridization with their respective mRNA LPN molecules, nor that the rate of hybridization for each oligonucleotide ECDP and its respective mRNA LPN, is free of nucleotide sequence related secondary structure inhibition effects. For SGDS oligonucleotide microarray gene comparisons, the presence of such nucleotide sequence related secondary structure inhibition of hybridization kinetics for a particular oligonucleotide ECDP, should not present a problem, as long as the particular mRNA LPNs compared represent the same portion of the mRNA of interest, and are close to the same nucleotide length, nucleotide sequence, and nucleotide composition. In this context, current oligonucleotide microarray protocols often provide a method for reducing the nucleotide length of the compared LPN molecules to around 80-300 nucleotides in length. Whether the compared reduced size LPNs always have the same length is not known.

For prior art microarray gene comparisons, assay PS-HKR values which deviate from one by 5-10 fold or more are plausible, but are likely rare, and will be associated with the effect of strong secondary structure on the hybridization kinetics of the LPNs. Prior art differences in compared LPN nucleotide length or complexity make plausible prior art assay PS-HKR values, which deviate from one by 1.5-2 fold. Such particular gene comparison PS-HKR assay values may not be uncommon. Note that very little experimental information exists concerning the existence of LPN nucleotide length or complexity related PS-HKR≠1 situations in prior art microarray assay gene comparisons. Only rarely does prior art microarray practice determine either the nucleotide length or nucleotide complexity of the compared LPNs.

For a cell sample expression comparison assay, different compared particular gene LPNs can be associated with different PS-HKR values. This can occur because of the differences in nucleotide lengths and/or nucleotide sequences and/or complexity, which are associated with different particular gene LPN comparisons in the assay. A variety of assay factors related to the isolation, purification, and processing of cell sample mRNA, and to the production of mRNA LPN molecules, are responsible for these differences. Because of these factors and the resulting differences, it was earlier concluded that it is likely that for many prior art particular gene comparisons, the assay PS-HKR is not equal to one. In addition, on the basis of these differences it was estimated that the assay PS-HKR values for a significant number of prior art gene comparisons deviate from one by 1.5-2 fold.

A PS-HKR assay value is also associated with each DGDS and SGDS particular gene comparison in an assay. For such comparisons, the nucleotide sequences of the compared LPNs are always significantly different, and the nucleotide compositions may be different. For such comparisons it is likely that secondary structure differences in the compared LPNs will be greater than for SGDS particular gene comparisons.

Not included in the above-described evaluation and estimate of the magnitude of the effect of the PS-HKR, is the effect of the label densities associated with the compared particular gene LPNs on the assay value for the PS-HKR for the comparison. For many prior art particular gene comparisons the LDR effect could further increase the assay PS-HKR value from the estimated 1.5-2 fold deviation from one, to an estimated 2-4 fold deviation from one. The LDR will be discussed later.

For a microarray particular gene comparison where a nucleotide sequence or composition related difference in LPN hybridization kinetics occurs, the difference can be corrected for if the assay PS-HKR is known. In contrast to microarrays, for properly designed non-microarray gene comparison methods such as northern blots, dot blots, nuclease protection, and RT-PCR, neither the PL-HKR, or PS-HKR is likely to be a factor.

The Assay Factor PSAR.

In a cell sample LPN preparation, different particular gene mRNA LPNs can have different PSA values. This can occur because of differences in the nucleotide sequence and composition of different particular mRNAs, or because of differences in nucleotide sequence and composition which occur in different regions of the same mRNA molecule. Whether such differences cause a difference in PSA values between different particular mRNA LPNs, depends on the method of producing and labeling the LPN. The PSA is quantified in terms of label signal activity per mass unit of a particular gene's mRNA LPN. Note that the PSA value for a particular gene's mRNA LPN, may or may not equal the TSA value for the cell sample total mRNA LPN preparation which it is part of.

For a microarray SGDS particular gene comparison, when the compared particular gene mRNA LPN molecules from each compared cell sample have the same, or nearly the same, assay value for the label signal activity per mass unit of the particular LPN, the assay PSA values for the compared LPNs will be the same. Thus, the assay PSAR=1. For a microarray SGDS particular gene comparison, the assay PSA value for one cell sample's particular gene mRNA LPN, can be different from the assay PSA value for the same gene mRNA LPN from the other compared cell sample. Put differently, the assay PSAR≠1 for the particular gene comparison. An assay PSAR≠1 value reflects differences in the label signal activity per mass unit of LPN values for the compared particular gene mRNA LPNs. Such differences can be caused by differences in the nucleotide sequence and/or nucleotide composition of the compared particular gene mRNA LPN molecules, or by differences in the efficiencies of labeling each cell samples mRNA LPN preparation, or both. As discussed earlier, assay factors related to the isolation, purification, and processing of cell sample mRNA, and to the production of mRNA LPN molecules, can cause such differences to occur. Because of these assay factors it is reasonable to believe that the assay PSAR≠1, for many prior art particular gene comparisons. Assay PSAR values, which deviate from one by 5-10 fold or more, are plausible, but should be relatively rare. Assay PSAR values which deviate from one by 2-4 fold, are likely not uncommon. Note that very little experimental information exists concerning the assay PSAR values. Prior art microarray practice does not determine the PSAR for each particular gene comparison, nor take the assay PSAR into consideration during the prior art normalization process.

On the basis of assay differences in compared particular gene mRNA LPN molecules which are known to occur, it was earlier indicated that assay PSAR values which deviate from one by 2-4 fold are probably not uncommon. Not included in this earlier evaluation and estimate, is the effect of the label density ratio, or LDR, on the assay PSAR value for a particular gene comparison. For many prior art particular gene comparisons, the LDR effect may further increase the deviation of the assay PSAR value from the estimated 2-4 fold from one, to roughly 3-8 fold deviation from one. The LDR effect, which is pertinent to the assay PSAR, is the fluorescence quenching effect. The LDR will be discussed later.

For a microarray particular gene comparison associated with mRNA LPN PSA differences, the PSA differences can be corrected for by the assay value for the PSAR for the particular gene comparison. In contrast, for properly designed non-microarray gene comparison methods such as northern blots, dot blots, nuclease protection, and RT-PCR, the PSAR should not be a factor.

A PSAR value is also associated with each DGDS and DGSS particular gene LPN comparison in an assay. For such comparisons the LPN nucleotide sequences are always different, and the LPN nucleotide compositions may be different. In addition, for such comparisons it is likely that the LPN secondary structure differences are greater than for SGDS comparisons.

The Assay Factor LLSR.

For a particular cell sample Type 2 total mRNA LPN preparation, all different particular mRNA LPNs have the same assay value for the LLN. The assay LLN value for a cell sample Type 2 total mRNA LPN molecule population, is defined in terms of the number of label signal molecules when are associated with each individual LPN molecule in the population. The assay LLS value for a cell sample Type 2 LPN molecule prep is defined in terms of label signal activity per LPN molecule.

For a particular cell sample Type 1 total mRNA LPN preparation, all particular gene mRNA LPNs may or may not have the same LLN or LLS assay value. For the vast majority of the prior art microarray gene comparisons, the assay LLN and LLS values are not the same for each particular gene mRNA LPN in a cell sample total mRNA LPN preparation.

In the event that assay LLS values are different in different compared cell samples for a Type 2 LPN cell sample gene comparison, then the difference can be corrected for with the assay LLSR value for the assay. The Type 2 assay value for the LLSR is the same for each particular gene comparison in the assay, and is therefore a global NF, and will affect all particular gene comparisons in the assay in the same way.

An LLSR value is also associated with each DGDS and DGSS particular gene LPN comparison in an type 2 LPN assay. For such comparisons the LLSR is a global assay UNF.

The Assay Factors LD, LDR, and PSSR.

The label density or LD of a particular genes mRNA LPN molecule population is measured in terms of the number, or average number, of direct or indirect label molecules per LPN base or nucleotide. For a particular gene comparison the ratio of, (the assay LD value for one cell sample's particular gene mRNA LPN)÷(the assay LD value for the other cell sample's same particular gene mRNA LPN), is termed the LD ratio, or LDR.

A directly labeled LPN molecule can be labeled directly with radioactive, fluorescent, chemiluminescent, phosphorescent, or some other signal generating label molecule. An indirectly labeled LPN molecule can be labeled with a label binding molecular entity such as, Biotin, a hapten, Avidin or some other protein, an oligonucleotide, or some other molecular entity, which can interact with and bind a signal generating molecule or entity. Prior art microarray and non-microarray gene comparison assays primarily utilize fluorescent or radioactive signal emitting molecules for direct labels, and Biotin and various Haptens for indirect labels. For microarray assays, fluorescence is by far the most widely used signal emitting label molecule, and Biotin is the most widely used label binding molecule. Therefore, for simplicity this discussion will focus primarily on fluorescence and Biotin direct and indirect labels. However, the discussion will apply directly to other direct and indirect labels as well.

In a cell sample total mRNA LPN preparation, different particular mRNA LPNs can have different LD values. For a cell sample LPN prep, the average number of label molecules per base for all of the LPN molecules which are present in the cell sample LPN prep, is termed the average label density or ALD. For a cell sample gene comparison, the ratio of, (the ALD value for one cell sample total mRNA preparation)÷(the ALD value for the other compared cell sample total mRNA LPN preparation), is termed the ALD ratio, or ALDR.

The PSA value for a particular gene LPN is measured in terms of the quantitative amount of label signal activity per microgram of LPN. Such a PSA value is readily converted to the quantitative amount of label signal activity per base of the LPN. The LD value for the same particular gene LPN is measured in terms of the number of label molecules per base of the LPN. Thus, for a particular gene LPN, the magnitude of the PSA value, and the hybridized LPN assay signal, will be directly proportional to the magnitude of the LD value. This will occur unless some other assay factor is affected by the label or the magnitude of the LD, and as a result the proportional relationship is changed. Such LD and label effects on other assay factors are herein termed LD effects. Such LD effects are considered to be negligible when the LD value is not associated with a significant change in the direct proportionality of the LD and the magnitude of the PSA and/or the hybridized LPN assay signal.

The assay characteristics of a particular gene's mRNA LPN can be affected in various ways by the LD (7, 30, 158, 161, 162). The LD may cause a slowing of the kinetics of hybridization of the LPN with the genes CDP. The LD can also affect whether the resulting hybridized LPN duplex is stable under assay conditions. Here, each labeled nucleotide in the LPN duplex is similar to a damaged or mismatched base. Herein the LPN duplex stability LD effect refers to the effect of the LD on the LPN duplex stability under assay conditions. These LD effects are essentially absent or minimal at low LD values, but can be very significant at high LD values. At high LD values, the LPN can lose its ability to hybridize stably with the CDP. At lower LD, the kinetics of hybridization of the LPN with the CDP can be slowed significantly, and the resulting LPN hybrids only partially stable. At an even lower LD, the LPN hybridization kinetics are unaffected, and the resulting LPN hybrid duplexes are completely stable. Under the usual assay conditions, for a particular gene LPN the LD related LPN hybridization kinetic slowing effect will occur at a much lower LD value than does the LPN duplex stability effect. At high LD values, these LD effects occur together. That is, when the LPN hybridization kinetics are slowed, the hybridized LPN duplex stability is reduced, and one effect magnifies the other. The assay manifestation of one or both effects is a smaller assay RAS value for the particular gene's LPN. The assay stringency of hybridization and posthybridization washing can greatly magnify or minimize the effect of the LD on the LPN hybridization kinetics and duplex stability. Further, the effect of the assay LD value for a particular gene mRNA LPN in an assay on the LPN hybridization kinetics and duplex stability, is likely to be much greater for an oligonucleotide array which has short ECDP molecules, than for oligonucleotide arrays with long ECDP molecules, or a cDNA array with even longer ECDP molecules.

The LD can also cause the reduction, or enhancement, of the signal activity per fluorescent molecule in a fluorescent LPN, thereby reducing or enhancing the signal activity of the LPN molecules (161, 162). At high LD, the LPN fluorescent signal can be reduced by fluorescence quenching due to the interaction of closely spaced dye molecules. Quenching can occur when the fluorescent LPN is in a double or single strand form. Quenching is absent or minimal at low LPN LD values, but can be quite significant at high LD values. Quenching generally occurs at LDs of less than one dye per 8 bases.

Depending on the particular fluorescent molecule type, which is present in the LPN, the signal activity per fluorescent molecule for the LPN can be reduced or enhanced by being in a single or double strand form. Such effects may or may not be related to the LD of the LPN. However, in certain instances the enhancement or reduction in the single or double strand state is observed only at particular LD values. Such effects are likely to be due to dye•nucleotide interactions.

The LD effects for different labels can be different. As an example, only at high LD values does Biotin affect the LPN hybridization kinetics and hybrid stability. In contrast, the widely used Cy3 fluorescent label has been reported to have an LD effect when the LD of the LPN is greater than one Cy3 molecule per 20 bases. The presence of the aminoallyl label in the LPN is also reported to affect the hybridization efficiency.

One of the important factors which determines the just detectable abundance, or JDA, for a particular gene LPN in an assay, is the label signal activity of the LPN. Herein, the label signal activity of LPNs has been described in different ways, and these include the TSA, PSA, and LLS. Generally, the higher the LPN signal activity, the lower the assay JDA which can be achieved for the LPN. As discussed earlier, the JDA for many prior art microarray gene comparison LPNs is inadequate to detect all, or even most, low abundance mRNAs in an assay. Because of this, microarray practitioners often try to maximize the label signal activity of the LPN preparations compared, by having as high an ALD for the LPN as possible. With regard to the Cy3 and Cy5 fluorophores, it has been reported that lower hybridization signals are obtained when greater than one dye molecule per 20 bases are present in a cell samples total mRNA LPN preparation (158). It is not uncommon for prior art cell sample Cy3 of Cy5 total mRNA LPN preparations to have ALDs around, or greater than, one dye per 20 bases. As an example, an Amersham document (2-20-02) describing Amersham's kit labeled Cy3 and Cy5 cDNA, indicates that: The CyScribe First Strand Labeling Kit produces Cy3 of Cy5 cDNA LPNs with an ALD range of from 1 dye molecule per 12 bases, to 1 dye molecule per 20 bases; the CyScribe Post Labeling Kit produces Cy3 cDNA LPNs with an ALD range of from 1 dye molecule per 13 to 30 bases, and Cy5 cDNA LPNs with an ALD range of from 1 dye molecule per 9 to 30 bases. Note that these ALD values are average values for the entire cell sample Cy3 or Cy5 mRNA LPN population. Consequently, a significant fraction of the particular gene mRNA LPNs which are present in the LPN preparation will have significantly higher LDs. Thus, in order to obtain the lowest assay JDA possible many prior art microarray assay gene comparison Cy3 and Cy5 LPNs have LDs which are near, or greater than, the LD of one dye per 20 bases which has been reported to cause a reduction of hybridization signal. The effect of these prior art assay LD values is magnified by the prior art practice for minimizing non-specific hybridization of the LPN during the assay. Prior art often performs the assay hybridization are posthybridization processes at as high a stringency as possible, in order to minimize the effect of LPN non-specific hybridization on the assay signals. This magnifies the LD effect on LPN hybridization kinetics and duplex stability, since at higher hybridization stringency the LD related slowing of hybridization kinetics can occur at a lower assay LD value for the LPN. Similarly, at higher hybridization and posthybridization process stringency, the LPN duplex stability effect will occur at a lower assay LD value. Significant hybridization kinetic slowing can occur before the LPN duplex stability in the assay is affected. The stringency of hybridization or posthybridization processes does not affect the LD related quenching. Quenching is generally believed to occur at the high assay LPN LD of about one fluorescent dye molecule per eight bases, or less. The available information suggests that it is not uncommon for prior art compared cell sample total mRNA LPN preparations, to have ALD values of 1 dye molecule per 10-20 bases. Such an LD value for a cell sample's total mRNA LPN preparation is the LD for the average LPN molecule in the preparation. Particular mRNA LPN molecules, which are present in the LPN preparation, can have much higher or much lower LDs. Consequently, many particular gene mRNA LPNs present in these total mRNA LPN preparations, are likely to have LD values at which fluorescent quenching will occur in the assay. Gene mRNAs, which are particularly rich in the nucleotide used to incorporate the dye, will have the highest LD values. Such genes can be identified by their nucleotide sequences. Note that the fraction of the total different mRNA LPN molecules which are present in an LPN preparation and which exhibits quenching may be low, but the actual number of genes involved may be high, since 12,000 or so different genes are expressed in a typical mammalian cell sample.

Prior art cell sample gene comparisons rarely measure, or report, the assay LD values for the compared cell sample total mRNA LPNs. Nevertheless, it seems likely that the LD related fluorescence quenching effect is not a major problem for many prior art particular gene mRNA LPN comparisons, but that for many others quenching is likely to be a problem for a significant number of genes in the assay.

The available information indicates that a particular directly labeled fluorescent LPN which is associated with the quenching LD effect is likely to be associated with the LD related hybridization kinetic slowing, and the LPN duplex stability reduction. Of these three LD effects, the hybridization kinetic slowing of an LPN is the most sensitive to the LD value. The kinetic effect will generally occur at significantly lower LD values than the LPN duplex assay stability effect, and the quenching effect. In effect, incorporating a label molecule into a polynucleotide molecule damages the hybridization capability of the LPN molecule in a manner analogous to the effect of a nucleotide sequence change by a point mutation, or by a damaged base. Each of these changes results in a weakened hybrid duplex. In this context, the effect of the presence of the label in the LPN can be regarded as a nucleotide sequence effect. As with the mismatched or damaged base pairs, the higher the LD, the greater the effect of the LD on the LPNs hybridization kinetics, and duplex stability (187). Available information indicates that for Cy3 and Cy5 total mRNA LPN preparations, an ALD value of 1 dye molecule per about 20 bases results in decreased hybridization, relative to an LPN preparation with a lower ALD value. In this situation where the ALD value of the LPN preparation is about one dye molecule per 20 bases, any effect of quenching on the overall hybridization signal should be small, and the decrease in hybridization signal is likely due to a general decrease in the LPN hybridization kinetics. As discussed earlier, the available information suggests that prior art compared cell sample total mRNA LPN preparations with assay ALD values of one Cy3 of Cy5 dye molecule per 10-20 bases, is not uncommon. For such comparisons it is likely that a significant fraction of the particular gene mRNA LPNs are associated with LD related hybridization kinetic slowing as well as quenching. Here the higher the assay LD value for the total mRNA LPN preparation, the greater the fraction of particular gene mRNA LPNs which is associated with the hybridization kinetic slowing effect.

At low LD values, quenching is essentially absent. However, even at low LD values other fluorescence related effects can cause a reduction or enhancement of an LPNs fluorescent signal activity per label molecule. Such effects may or may not be related to the LD of the LPN. In certain instances, the signal activity per fluorescence molecule for an LPN can be different, depending on whether the LPN is in a single or double strand state. That is, whether the LPN is hybridized or not (161). Here, the signal activity per dye molecule for one dye type LPN may be enhanced by hybridization, while the signal activity per dye molecule of an LPN labeled with a different dye may be reduced by hybridization. Such signal activity behavior would not be related to the LD. In another instance, the enhancement or reduction of an LPNs fluorescent signal activity is related to the LPN LD value. As an example, at a particular Cy3 LD value an LPN's signal activity per fluorescent molecule is greater when the Cy3 LPN hybridized, than when the Cy3 LPN is single stranded. At another Cy3 LD value an LPN's signal activity per fluorescent molecule is less when the Cy3 LPN is single stranded or non-hybridized, than when the Cy3 LPN is double stranded or hybridized. In a cell sample total mRNA Cy3 LPN preparation, different particular gene mRNA Cy3 LPNs can have significantly different nucleotide sequences and nucleotide compositions. Because of this, it seems plausible that one or more particular mRNA Cy3 LPNs can exhibit enhanced signal activity in the hybridized state, while one or more different particular mRNA Cy3 LPN can exhibit reduced signal activity in the hybridized state. Reports of such Cy3 LPNs, or Cy5 LPNs, have not been discovered.

As described above, the assay LD value for the LPN can affect the LPN hybridization kinetics, the hybridized LPN duplex stability, and the LPN's signal activity per label molecule. The LD related hybridization kinetic effect can be characterized as a nucleotide sequence and/or composition effect, and therefore can be described as a particular sequence determined hybridization kinetic effect, or a PS-HK effect. The LD related signal activity effect also can be characterized as a particular sequence determined effect, and therefore can be described as a particular nucleotide sequence determined signal activity effect, or a PSA effect. The LD related LPN duplex stability effect may also be characterized as a particular nucleotide sequence determined effect, and can be described as a particular sequence duplex stability effect, or PSS effect. Herein, the PS-HK and PSA effect categories have been earlier described, while the PSS effect has not. Thus, the effect of the LD values of compared particular gene mRNA LPNs on the assay RASR value for the particular gene LPN comparison, can be discussed in terms of the earlier described PS-HKR and PSAR assay values, and the just described PSS ratio, or PSSR. Herein, the PSS for a particular mRNA LPN is expressed in terms of the fraction of the LPN which can form a stable hybridized duplex with the CDP during the assay, relative to the fraction of the same LPN not associated with LD effects, which can form a stable hybridized duplex with the same CDP. The PSSR is then equal to the ratio of (the PSS for one cell samples particular gene mRNA LPN)÷(the PSS for the other cell samples same particular gene mRNA LPN). The PSSR can be different for different particular gene comparisons, and is a non-global assay variable NF. PSS and PSSR values are difficult to measure.

Fluorescent signal generation molecules are by far the most frequently used labels for microarray gene comparisons. Next most frequently used are radioactive label molecules. The vast majority of prior art microarray gene comparisons utilize either fluorescence or radioactive LPNs. Relative to fluorescence, there are far fewer LD related effects for radioactive LPNs. The radioactive signal can be quenched, but this is easily avoided. Absent quenching effects, there is no difference in the radioactive signal activity per radioactive molecule for hybridized or non-hybridized LPNs. Further the LD effect on the LPN hybridization kinetics and duplex stability, can only be caused by the radiation induced damage of the LPN, and/or the resulting reduction in the induced damage of the LPN, such as base damage or strand scission. This can be readily avoided. Thus, from the point of view of LD effects, radioactive labels are preferable to fluorescent labels.

An LDR value is also associated with each DGDS and DGSS particular gene LPN comparison.

The Association of Signal Generation Complexes with Hybridization Immobilized Indirectly Labeled LPNs: the Assay Factors SBNR and SSAR.

By themselves, hybridization immobilized indirectly labeled LPN molecules used in microarray and non-microarray assays are not associated with a directly detectable signal, which can be used to detect and quantitate the presence of such indirectly labeled LPN molecules. Therefore, in order to detect and quantitate the presence of the hybridization immobilized indirectly labeled LPN molecules, it is necessary to stably and rationally associate one or more signal generating complex molecules (SGCs) with each ligand containing hybridization immobilized LPN molecule. Combinations of indirect ligand labeled LPNs and SGCs are commonly used in the prior art. Commercial microarray systems from GE, Affymetrix, and Applied Biosystems, use such an approach. Affymetrix uses Biotin labeled LPNs and a streptavidin-phycoerythrin SGC, while GE uses Biotin labeled LPNs and streptavidin-Cy5 SGC. Applied Biosystems uses Digoxigenin (DIG) labeled LPNs, and an SGC composed of anti-DIG antibody-alkaline phospatase SGCs. Other ligand-SGC combinations which are available are: Invitrogen's Biotin and anti-Biotin antibody covered gold particles which are detected by light scattering; Genisphere's 3DNA fluor-DNA dendrimer complexes which bind to the array immobilized LPN by a specific hybridization reaction; Martek's Biotin and streptavidin conjugated phycobiloproteins; and Quantum Dot's Biotin and streptavidin coated fluorescent quantum dots. Each of these SGCs has a characteristic molecular size or approximate diameter. In addition, depending on the particular assay, the average nucleotide length of the indirectly labeled cell sample LPN molecules used in the assay ranges from about 35 mm, or 100 bases long, to about 500 mm, or 1500 bases long. These are summarized in Table 18. A variety of other ligand-SGC combinations are available. However, the above-noted combinations are generally representative of such other combinations.

TABLE 18
Types of Signal Generation Complexes (SGC) Associated
with Indirectly Labeled LPNs

| | Approximate | |
|---|---|---|
| Type of Signal Generation | Molecular | Average^{(a)} Nucleotide |
| Complex (SGC) Associated | Diameter of | Length of Hybridization |
| with Indirect Labeled LPNs | SGC (nm) | Immobilized LPN (nm) |
| Streptavidin - Phycoerythrin^{(b)} | ˜15 | ˜35 |
| (SA·PE) (Affymetrix) | | |
| Biotinylated Anti-Streptavidin | ˜15 | ˜35 |
| Antibody (Affymetrix) | | |
| Streptavidin - Cy5^{(c)} (SA·Cy5) | ˜5 | ˜35 |
| (GE) | | |
| Alkaline Phosphatase - Anti- | ˜20 | Varies ˜50-500 |
| Digoxigenin Antibody (Applied | | |
| Biosystems) | | |
| Streptavidin Coated Quantum | 20 or 40 | Varies ˜50-500 |
| Dots (Quantum Dot Inc.) | | |
| Streptavidin Conjugated | 50 or 80 | Varies ˜50-500 |
| Phycobiloprotein Complexes | | |
| (Martek) | | |
| Anti-Biotin Antibody Coated | 110 | Varies ˜50-500 |
| Gold Particles (Invitrogen) | | |
| 3DNA Dendrimer Fluor | 200-300 | Varies ˜50-500 |
| Complex (Genisphere) | | |

^{(a)}A 100 base long DNA molecule is ˜15-35 nm long.
^{(b)}Each SA·PE complex contains on PE molecule and 2-3 SA molecules.
^{(c)}Each SA·Cy5 complex contains one SA molecule and 2-4 Cy5 molecules.

For gene expression analysis assays which compare indirectly labeled cell sample LPNS, the ligand molecules are attached directly to the LPN molecules, and when an LPN molecule is immobilized by hybridization to the spot, the ligand is also immobilized to the spot surface. Here, an LPN molecule indirectly labeled with one or more ligand molecules is termed a ligand-LPN molecule, or L-LPN molecule. In order to be able to detect an immobilized L-LPN molecule, one or more SGC molecules must be stably associated with the immobilized L-LPN molecule. This association must be stable and specific for the ligand associated with the LPN. For simplicity, the association of the SGC with the immobilized L-LPN molecule is termed the SGC binding reaction, or SB reaction. For a cell sample L-LPN assay, each particular gene comparison in the assay involves at least two binding steps. The purpose of the SB reaction is to associate signal generation molecules with the spot hybridization immobilized L-LPN molecules so that they can be detected and quantitated. In order to detect quantitate and compare the absolute or relative number of L-LPN molecules which are associated with a particular gene spot, a predictable absolute or relative quantitative relationship between the number of immobilized particular gene L-LPN molecules in a spot, and the assay measured signal activity associated with the spot immobilized SGC molecules, must be known.

Prior art assays which compare cell sample L-LPNs involve the following steps. (i) Produce the cell sample L-LPNs. Such cell sample L-LPN preps are produced in essentially the same manner as cell sample directly labeled LPN preps. The discussions on the production and characteristics of these LPNs apply directly to the L-LPNs. Generally, each compared L-LPN prep is indirectly labeled with the same ligand. While it is possible to utilize a different ligand label for each compared cell sample L-LPN prep, this is not often done, and this discussion will emphasize only the use of one ligand for a comparison, unless otherwise noted. However, the discussion will also apply to those assays using two different signal labels and ligands. A very large fraction of prior art indirect labeled LPN assays involve Affymetrix or GE commercial assays. Therefore, this discussion will be in terms of these assays. For both of these assays it has been reported that the cell sample Biotin labeled cRNA molecules contain about one Biotin molecule per 10 bases. In addition, for both these assays the compared cell sample L-LPN preps are fragmented to a smaller size before the hybridization step. As indicated in Table 18 the average cell sample L-LPN molecule fragmented nucleotide length is reported to be about roughly 100 bases for Affymetrix and GE assays. Prior art only rarely precisely determines the nucleotide lengths of either the synthesized or fragmented compared cell sample cRNA L-LPN preps. Further, prior art does not take such nucleotide lengths into consideration for normalization. The above-described cRNA L-LPNs are Type 1 L-LPNs. (ii) Each compared fragmented cell sample L-LPN prep is hybridized to a separate microarray under controlled conditions. Non-hybridized cRNA L-LPN molecules are then removed from the microarray. (iii) Each compared microarray is then incubated with an aliquot of one stock SGC staining solution in order to bind SGC molecules to the hybridization immobilized cRNA L-LPN molecules. Here, each compared microarray is exposed to the same SGC staining solution, and therefore the SGC molecules which bind to each microarray should have identical in solution signal activity properties. Here, any observed difference in the basic signal activity properties of the immobilized SGC molecules on one compared slide, relative to the other, are believed to be caused by differences in the hybridized microarrays. Non-immobilized SGC molecules are removed from each microarray with a wash step. To this point, the Affymetrix (190) and GE (184) assay steps are essentially identical. From this point, the GE protocol is significantly simpler, and while the discussion will primarily focus on the GE method, it applies as well to the Affymetrix method. (iv) For each compared microarray, the total spot signal (TSS) is measured for each particular gene spot. Identical signal generation and detection conditions are used to measure the signal activity associated with each particular gene spot on each compared microarray. Further, the SGC molecules used in the SGC binding step for each compared cell sample microarray are believed to be identical. Therefore, any observed difference in the basic signal activity properties of the immobilized SGC molecules associated with one cell sample's particular gene spot on one microarray, relative to the same particular gene's spot on the other cell sample microarray, can be attributed to differences in the microarray spots themselves. Such differences may be related to differences in spot surface environments caused by physical, chemical, or charge differences in the compared spot oligonucleotides or surfaces. (v) The background signal is subtracted from each compared particular gene TSS value to produce a raw assay signal value or RAS, for each particular gene in the comparison, and an RASR value for each particular gene comparison. (vi) The particular gene RAS and/or RASR values are then normalized for prior art considered assay variables to produce particular gene NASR values. Prior art believes and practices that such prior art indirect label assay measured particular gene NASR values, are biologically accurate.

The quantitative signal activity associated with each gene's spot immobilized L-LPN molecules is dependent on a variety of assay factors which affect either the number of SGC molecules which can stably bind to the spot immobilized L-LPN molecules, or the efficiency of signal generation and detection for the immobilized SGC complexes present in the spot. Here, a measure of the number of SGCs which can stably bind to a hybridization immobilized particular gene L-LPN molecule, is termed the SGC molecule binding number, or SGC binding number, or the SBN. For a particular gene comparison, the ratio of the compared particular gene SBN values is termed the SBNR. The SBN for an immobilized L-LPN molecule reflects the number of SGC molecules, which can bind to, and stably associate with, an immobilized L-LPN molecule. The SBN for an immobilized L-LPN molecule of a particular nucleotide length or average nucleotide length, can be expressed in terms of the number of stably bound SGC molecules per nucleotide for the L-LPN molecule. The SBNR can be expressed as the ratio of the absolute SBN values, or relative SBN values, for the compared immobilized L-LPN molecules. For an immobilized L-LPN of known nucleotide length, (the signal activity associated with the L-LPN molecule)÷(the L-LPN nucleotide length in nucleotides), is a relative measure of the SBN value for the L-LPN. The ratio of the signal activity per nucleotide values for different compared immobilized L-LPN molecules of known nucleotide lengths, is a measure of the SBNR for the L-LPN comparison. In an assay, compared immobilized L-LPN molecules which have the same nucleotide lengths and are associated with the same number of SGC molecules, and will also have the same signal activity per nucleotide value, and the comparison SBNR value equals one. Prior art does not determine or consider for normalization, the UNF SBNR. The SBNR UNF is associated with non-global assay variables. Further, the SBNR is pertinent only for cell sample Type 1 L-LPN comparisons.

The efficiency of signal generation and detection of the spot immobilized SGC molecules is measured in terms of the amount of signal activity detected per SGC molecule. Here, the quantitative amount of signal activity per immobilized SGC molecule, is termed the SGC molecule signal activity, or SSA. For a particular gene comparison, the ratio of the compared particular gene SSA values, is termed the SSAR. Prior art does not determine or take into consideration during normalization, the SSAR. For L-LPN comparisons which use only one SGC type, it is generally reasonable to believe the assay SSAR value is equal to one. The SSAR UNF is not pertinent to Type 2 L-LPN comparison assays. The SBN, SBNR, SSA, and SSAR will be discussed primarily in the context of the GE codelink indirect label LPN comparison method. Again, this discussion will apply directly to the Affymetrix system, as well as others. Both of these assays utilize Type 1 L-LPNs.

A variety of factors can affect the magnitude of the SBN value for a particular gene spot immobilized L-LPN molecule. These include, but are not limited to, the following. (i) The molecular dimensions of the SGC molecule used. The GE codelink GSC molecule is a streptavidin-Cy5 (SA•Cy5) complex which has an almost square molecular shape with dimensions of about 5 nm×5 nm×5 nm. Each SA•Cy5 molecule contains 2-4 Cy5 molecules. (ii) The ligand label density along the L-LPN molecule. Here, as with the Affymetrix system, there is on average, about 1 Biotin present per every 10 nucleotides in the L-LPNs. This is equivalent to about one Biotin molecule for every 3 nm of L-LPN length for a stretched out single DNA strand. (iii) The nucleotide length of the immobilized particular gene L-LPN. For the GE system, the average cRNA L-LPN length is about 100 bases long when fully stretched out. Such a stretched out L-LPN molecule has a nucleotide length of about 35 nms, and contains about 10 Biotin molecules, each on average about 3-4 nm apart. The maximum number of SA•Cy5 molecules, which may bind to a fully stretched out L-LPN molecule, is about 7. That is, for a fully stretched out L-LPN molecule in the single or double strand state, the SBN value is about 0.07. In reality, the actual SBN is likely to be lower for the GE assay situation because: only a maximum of 30 of the hybridization immobilized L-LPNs 100 bases can be in the double strand form since the immobilized CDP molecule is only about 30 bases long and the single strand portion of the immobilized L-LPN molecule will not be fully stretched out due to the formation of salt induced intrastrand secondary structure in the staining step. On average, such immobilized single strand regions have a nucleotide length of about 35 bases, and a secondary structure induced diameter of roughly 4 nm or so. The close spatial proximity of the Biotins in the 4 nm rough sphere, and the ability of a single SA molecule to bind multiple Biotins, would limit the number of SA•Cy5 molecules which could bind to the single strand regions of the immobilized L-LPN molecule. A reasonable estimate would be an SBN of 0.03 to 0.05 for a 100 base long immobilized L-LPN. For comparisons of cell sample L-LPN preps the SBNR assay value should be equal to one when the compared cell sample L-LPNs have the same nucleotide lengths, and the same Biotin label density. Significant differences in the Biotin label densities and/or nucleotide lengths for compared cell sample L-LPN preps can cause the SBNR values for compared particular gene L-LPNs to deviate significantly from one. Absent other compensating factors, such a deviation will cause the particular gene RASR value to significantly deviate from the particular gene ACR value. Prior art GE or Affymetrix assays rarely determine the nucleotide length and the Biotin label density of the compared cell sample fragmented cRNA L-LPN preps. It seems reasonable to believe that compared fragmented cRNA nucleotide length differences of twofold would not be unusual, and that significant Biotin label density differences also occur. Note that while a twofold difference in compared L-LPN nucleotide lengths will significantly affect the GE and Affymetrix assay SBNR value, a twofold difference in the ligand density for the compared L-LPNs may not affect the SBNR significantly in certain situations. (iv) The kinetics of binding of the SA•Cy5 molecules with the spot immobilized L-LPN molecules. Here, the SGC binding step should, if possible, be designed so that the binding reaction is completed in only a fraction of the binding period in order to eliminate the effect of any binding kinetic differences which exist for individual binding steps for compared cell samples. Note that since identical populations of SA•Cy5 molecules are generally used to stain compared arrays, any binding kinetic differences which exist for an assay are almost certainly associated with one or more differences in the compared array spot surfaces. There is essentially no prior art information available on this issue for the GE or Affymetrix assays. Absent other compensating assay design or assay variable factors, significant binding kinetic differences for compared cell sample particular gene L-LPNs can cause a particular gene comparison SBNR to deviate significantly from one, and the assay measured particular gene RASR value to deviate significantly from biological accuracy. (v) The stability of the SA•Cy5 or SA•PE LPN complex, once it has formed. Little or no information concerning this issue is available for the GE, Affymetrix, or other assay systems. Here, significant differences in the complex stability for compared particular gene L-LPNs can cause a particular gene comparison SBNR value to deviate significantly from one, and the assay measured particular gene RASR value to deviate significantly from the ACR and biological accuracy. Note that since identical populations of SA•Cy5 molecules are generally used to stain compared arrays, any binding stability differences which occur are almost certainly due to differences associated with the different arrays, or array spots. For differences in compared binding kinetics and/or binding stability, such differences may be caused by differences in the spot surface or content or the availability or accessibility of the immobilized Biotin. As an example, differences in the immobilized oligonucleotide CDP molecule density in the compared particular gene spots could cause differences in both the binding kinetics and binding stability.

A variety of factors can affect the magnitude of a particular gene SSA assay value for an immobilized SGC molecule. These, include, but are not limited to, the following. (i) The type of signal molecule which is associated with the SGC. As discussed, for the GE assay Cy5 fluorescent molecules are used, while for the Affymetrix assay fluorescent phycoerythrin protein molecules are used, and for ABI an enzyme chemiluminescent substrate system is used. (ii) The number of signal molecules associated with the immobilized SGC molecule. For the GE assay, about 2-4 Cy5 molecules are associated with each SA molecule, while for the Affymetrix assay, about 30 fluorescent dye molecules are associated with each SA•PE molecule, and for the ABI assay system there is one enzyme molecule associated with three identical anti-DIG FAB antibody fragments. Note that for the Affymetrix assay, three different binding reactions are used to produce a multi-layer immobilized SGC complexes, and multiple SA•PE molecules may be associated with each SGC complex. (iii) The conditions of signal generation and detection. For a cell sample cRNA L-LPN comparison, differences in i, ii, or iii, can cause a particular gene SSAR value to deviate significantly from one, and absent other compensating factors, cause the assay measured particular gene RASR value to deviate significantly from the ACR. However, while assay SSAR values are not determined by the prior art, it is reasonable to believe that for the great majority of prior art GE and Affymetrix assay measured RASR values, the SSAR values are equal to one or nearly one. This occurs because the compared arrays are stained with identical populations of SGC molecules taken from one stock solution, and the conditions of signal generation and detection are the same for each compared cell sample array.

Prior art does not determine or consider the SBNR values for particular gene comparisons of cell sample L-LPNs. Prior art practices and believes, that these particular gene comparison SBNR values are equal to one. However, it is known that the nucleotide lengths of compared particular gene L-LPN molecules and the Biotin label density of the synthesized cRNA L-LPNs can vary significantly, and in such a case the particular gene SBNR value could deviate significantly from one. Prior art only rarely determines, and does not take into consideration during normalization, the nucleotide lengths of the compared cDNA or c-RNA L-LPNs, or their actual Biotin label densities. In addition, the SGC binding kinetics and the stability of the immobilized SGC complexes is not determined or taken into consideration by the prior art. It is known that separate but replicate arrays, which are stained with the same SGC solution and measured under identical signal generation and detection conditions, can have very different, fourfold or greater, total signal intensities. Some prior art practitioners reject array comparisons associated with greater than threefold total intensity difference for the compared arrays. Affymetrix suggests that such differences may be, in part, due to differences in staining efficiencies for compared arrays. Affymetrix assumes, as do others, that such staining efficiency differences are solely associated with one or more global assay variables, and that the method of total intensity normalization (TIN), can be validly used to normalize compared arrays for such differences. As discussed here elsewhere, the use of the TIN method cannot be known to be valid for many, if not most, of these array comparisons. In addition, it cannot be assumed that any staining differences which occur are associated only with global assay variables, and not with non-global assay variables.

The GE assay uses only one SGC binding step, while there are three separate binding steps involved with the Affymetrix method of associating the SA•PE complexes with the spot immobilized cRNA L-LPN molecules. In addition, the Affymetrix method involves the use of two different ligand-receptor combinations, SA•Biotin, and anti-SA antibody and SA. Further, both the SA•PE and anti-SA antibody molecules are much larger than the SA•Cy5 molecules used for the GE method. The SA•PE complex consists of 2-3 SA molecules attached to a phycoerythrin molecule, and has a molecular weight of 340,000 to 400,000, and a molecular diameter of about 15-20 nm. The Biotinylated anti-SA antibody molecule has a molecular weight of about 150,000 and has effective molecular diameter of about 15 nm. In contrast, the SA•Cy5 complex has a molecular weight of about 53,000 and a molecular diameter of about 5 nm. Each of the three Affymetrix binding reactions is associated with binding kinetics and binding stability factors. The complexity of this staining step method makes it much more likely that the assay SBNR for a particular gene cRNA L-LPN comparison will deviate significantly from one than is the case for the GE assay method.

For the GE and Affymetrix assays, the earlier discussed TPN for particular gene cRNA L-LPN comparisons is greater than one and is often equal to greater than 5. Both GE and Affymetrix assays employ short, 25-30 nucleotide long oligonucleotide molecules, as immobilized CDP molecules, and the average nucleotide length of the fragmented cRNA L-LPNs is about 100 nucleotides. ABI uses 60 base long oligonucleotides as immobilized CDPs, and does not fragment the compared cRNA L-LPNs, which are generally roughly 500 nucleotides long. For all of these systems, only one cRNA L-LPN molecule can hybridize to a single immobilized CDP oligonucleotide molecule. Here, the longer the cRNA L-LPN molecule, the greater the number of SGCs which can be associated with a hybridization immobilized cRNA L-LPN molecule. However, it is not clear whether the increase in the number of bound SGC molecules with cRNA nucleotide length, is directly proportional to the increase in nucleotide length. This must be determined for each system in order to properly normalize the assay measured particular gene RASR values for differences in compared cell sample cRNA L-LPN nucleotide lengths.

For the GE, Affymetrix, and ABI assays, the particular gene comparison SSAR is pertinent, while the PSAR is not pertinent for the assay. As mentioned, it is reasonable to believe that the SSAR value for the GE and Affymetrix assays are equal to one. This cannot be assumed for the ABI assay because the enzyme activity and substrate availability are likely to be differentially affected by differences associated with the array surface, charge, and structure.

The GE, Affymetrix, and ABI cRNA L-LPNs are Type 1 LPNs and behave in the assay as Type 1 LPNs. While not employed for these assays, Type 2 L-LPNs can also be used. Type 1 and Type 2 LPNs were described earlier, and were primarily discussed in terms of directly labeled LPNs. When L-LPNs are compared in an assay, an indirectly labeled Type 1 L-LPN behaves as a Type 2 LPN under certain circumstances. This can occur when the molecular diameter of the SGC molecules used in the assay is significantly greater than the nucleotide length of the hybridization immobilized L-LPN molecule. In such a circumstance, only one SGC molecule may bind to each immobilized L-LPN molecule. Each immobilized L-LPN molecule is then associated with the same number of signal generating molecules, just as Type 2 LPNs are. If the SGC molecular diameter is much greater than the immobilized L-LPN nucleotide length, then one SGC molecule may bind with multiple immobilized LPNs.

For an L-LPN comparison assay, the SGC molecular size and L-LPN nucleotide length must be known in order to know whether the compared L-LPNs behave as Type 1 or Type 2 LPNs, and in order to properly identify and normalize for assay variables associated with the SGC and L-LPN combination. For example, an L-LPN which is produced as a Type 1 L-LPN, may behave in the assay as a Type 2 L-LPN if the molecular diameter of the SGC is similar to or somewhat larger than the nucleotide length of the immobilized L-LPNs in the assay, and the LPN TPN equals one. Here, the SBNR for each particular gene comparison in the assay can be ignored during the normalization. If the SGC is significantly smaller than the nucleotide length of the L-LPNs, then the Type 1 L-LPN behaves as a Type 1 LPN and the SBNR may or may not equal one for each particular gene comparison in the assay, and must be determined. In a situation where the SGC is very large relative to the L-LPN molecule, each SGC may bind to one or more L-LPN molecules. Here, it will not be possible to know how many immobilized L-LPN molecules an SGC is associated with, and it will not be possible to validly compare the signal magnitudes of the compared particular gene RAS values. Such a situation could occur with either relatively short or relatively long L-LPN molecules.

The unconsidered assay variable NFs SBNR and SSAR are associated only with indirect label cell sample L-LPN prep comparisons. For either of these UNFs, a significant deviation of an assay particular gene UNF value from one can cause the assay measured particular gene RASR value to deviate significantly from its ACR value, and from the biologically accurate value. It is reasonable to believe that prior art particular gene SBNR values which deviate from one by 1.5 to 3 fold are not uncommon. It is also reasonable to believe that most prior art particular gene SSAR values do not deviate significantly from one. It is likely that the prior art particular gene UNF SBNR values are associated with one or more non-global assay variables, while the SSAR UNF is associated only with global assay variables. Prior art does not determine either the assay SBNR or SSAR values.

An SBNR or SSAR value is associated with each DGDS and DGSS particular gene comparison in an assay.

Effect of TSAR, PSAR, and LLSR, Assay NFs on the Relationship (NASR)=(ACR).

The TSAR is a prior art considered assay variable NF. The TSAR has been included here because the prior art microarray practice often does not determine the TSAR, or normalize for the differences in the compared cell samples total mRNA LPN assay values. TSAR and PSAR NF values are measured in terms of label signal activity per microgram of LPN, and are applicable to microarray comparisons of Type 1 LPN preparations. The TSAR is a global NF, and the PSAR is a non-global NF. The LLSR is a global NF for Type 2 LPN comparisons, and is measured in terms of label signal activity per LPN molecule.

The effect of either the TSAR, PSAR, or LLSR, NF values on the (assay NASR)=(AHCR) relationship is presented in Table 19. In order to demonstrate the effect of each of these individual NF factors on this relationship for a particular gene comparison, it is assumed that the only assay variable which influences the assay NASR value, is the assay TSAR, or PSAR, or LLSR.

TABLE 19
Effect of TSAR, PSAR, or LLSR on the Relationship (Assay NASR) = (ACR)

| | | | | | ^{(a)}Gene A | | |
|---|---|---|---|---|---|---|---|
| | | | | Cell Sample | LPN Signal | | |
| Gene | | Compared | ACR | Gene A LPN | Activity | | Ratio of |
| Compared | | Cell | of | Signal | Ratio In | ^{(b)(c)}Observed | (Assay NASR) |
| in Assay | | Sample | Assay | Activity | Assay | Assay NASR | (ACR) |
| (i) | A | 1 | 1 | 1 | 1 | 1 | 1 |
| | A | 2 | | 1 | | | |
| (ii) | A | 1 | 1 | 4 | 4 | 4 | 4 |
| | A | 2 | | 1 | | | |
| (iii) | A | 1 | 1 | 1 | 0.25 | 0.25 | 0.25 |
| | A | 2 | | 4 | | | |
| (iv) | A | 1 | 4 | 1 | 1 | 1 | 0.25 |
| | A | 2 | | 4 | | | |
| (v) | A | 1 | 100 | 1 | 0.2 | 20 | 0.2 |
| | A | 2 | | 5 | | | |

^{(a)}Signal activity ratio can be TSAR, PSAR, or LLNR.
^{(b)}It is assumed that the only assay variable NF, which affects the assay NASR, is either the TSAR, PSAR, or LLSR.
^{(c)}All ratios have the cell Sample 1 parameters in the numerator.

Under these conditions the (assay NASR)=(ACR), for a particular gene comparison, when the TSAR or the PSAR or the LLSR, is equal to one. Table 19 illustrates that a difference in the compared cell sample LPN molecules TSAR, PSAR, or LLSR values, causes the (assay NASR)≠(ACR). In addition, the further the assay value for TSAR, PSAR, or LLSR, deviates from one, the greater the deviation of the assay NASR from the ACR. Note that such a deviation can cause a particular gene comparison assay result to be associated with an RDM.

The Effect of the Label Density Ratio (LDR) on the Relationship (Assay NASR)=(ACR).

Because the great majority of prior art gene comparisons utilize fluorescence as a label, this discussion will focus primarily on the effect of the LD of fluorescent LPN molecules, but will generally apply to other labels.

For the microarray gene comparison of a particular gene LPNs, the assay LD value for each compared LPN determines the assay LDR value for that particular gene comparison. For a particular gene comparison an LDR=1 value does not mean that there is no LD related effect on the relationship, (assay NASR)=(ACR). This is true even for SGDS gene comparisons which utilize only one label molecule. One reason for this is that the LD related effects are influenced by other non-LD assay factors. The assay LD for a particular gene mRNA LPN, is partly determined by the overall LPN labeling efficiency which is associated with the process of producing the cell sample total mRNA LPN preparation which contains the particular gene mRNA LPN. Both the assay values for the TSA and ALD reflect the overall labeling efficiency of the cell sample total mRNA LPN preparation. The TSA value is expressed in terms of label signal activity per mass unit of the cell sample total mRNA LPN preparation. The TSA represents the average label signal activity value for all of the particular mRNA LPNs which are present in the cell sample total mRNA LPN preparation. Put differently, the assay TSA is a measure of the average of the PSA values for all of the particular gene mRNA LPNs which are present in the cell sample total mRNA LPN preparation. Thus, the assay LD for a particular gene mRNA LPN reflects the assay PSA value for that particular mRNA LPN. In addition, both the assay PSA and LD values for a particular mRNA LPN, can be influenced by the nucleotide sequence and nucleotide composition of a particular mRNA LPN. Since the nucleotide sequence and/or composition is different for different particular mRNAs in a cell, and is also different for different regions of the same particular mRNA sequence, different particular mRNA LPNs in a cell sample total mRNA LPN preparation will have different PSA assay values and different LD assay values.

The assay values for PSA and LD for a particular gene mRNA LPN are influenced by, the overall labeling efficiency of the cell LPN preparation and the nucleotide sequence, and/or nucleotide composition of the particular gene mRNA or mRNA sub-region which produces the LPN. The overall labeling efficiency affects all particular mRNA LPNs in the cell sample LPN preparation in the same way, and is therefore a global assay variable. In contrast, the nucleotide sequence and/or nucleotide composition of different particular mRNA LPNs can be different, and therefore represent a non-global assay variable.

Because the overall LPN labeling efficiency, and the nucleotide sequence and/or nucleotide composition affect the magnitude of the assay LD value for a particular mRNA LPN, these factors also influence all LD related assay effects. These LD effects include the LD related hybridization kinetic slowing and the stability of the hybridized LPN duplex, and the LD related enhancement or reduction of LPN signal activity.

The LD related signal activity reduction and enhancement effects, including fluorescent quenching, is influenced by the above discussed two factors, and the label type. Each of these factors affects a particular LPN's assay PSAR value, and can therefore affect the relationship, (assay NASR)=(ACR). This relationship will be affected by these LD related effects to the extent that these LD related effects cause the assay PSAR to deviate from one. The effect of PSAR≠1, was discussed earlier (see Table 19). Thus, if the assay value for PSAR is known for a particular gene mRNA LPN comparison, the particular gene assay NASR can be corrected or normalized for LD related signal activity reduction and enhancement effects.

In addition, to the overall LPN labeling efficiency factor and the nucleotide sequence and/or nucleotide composition factor, the LD related hybridization kinetic effect is influenced by other non-LD assay factors. For a particular mRNA fluorescent LPN with a given assay LD, the LD related hybridization kinetic effect can vary with the type of fluorescent label, the LPN nucleotide length, the TCN and TPN of the LPN, the assay hybridization and posthybridization stringency conditions, and the particular LPNs ECDP. Each of these other factors and the LPN nucleotide sequence and nucleotide composition factor, can affect the particular LPN's assay PS-HKR value, and therefore can affect the relationship (assay NASR)=(ACR). This relationship will be affected by these LD related hybridization kinetic effects to the extent that these LD related effects cause the assay PS-HKR value to deviate from one. The effect of a PS-HKR≠1 assay value is discussed in a later section. Thus, if the assay value for PS-HKR is known for a particular gene mRNA LPN comparison, the particular gene assay RASR can be corrected or normalized for LD related hybridization kinetic effects.

The LD related hybridized LPN stability effect is related to the LD related LPN hybridization kinetic slowing effect. The stability effect will generally occur only after significant hybridization kinetic slowing has occurred. From that point, the stability effect will become greater as the kinetic effect increases. While the LPN hybrid stability effect is related to the hybridization kinetic effect, the LD related hybrid stability effect on the (assay NASR)=(ACR) relationship for a particular gene mRNA LPN comparison, cannot be corrected for by the particular gene comparisons PS-HKR assay value. The particular gene comparison's assay PSSR assay value must be used for this correction. In order to make this correction it is necessary to determine the PSSR assay value and use it for the correction, or normalize indirectly for the LPN hybrid stability effect on the assay RASR. Prior art microarray practice does not determine the assay PSSR for a particular gene comparison. Nor does prior art practice correct the assay NASR for the LPN hybrid stability effect. PSSR values are difficult to determine for even one particular gene comparison and it is not practical to determine the PSSR value for more than a very few such particular gene comparisons in an assay.

For a microarray cell sample gene comparison assay, different particular gene comparisons can be associated with different assay values for PSSR, the LD related portion of the PS-HKR, and the LD related portion of the PSAR. Therefore, each of these is a non-global assay variable.

For a particular gene comparison, the assay PSSR NF value affects the relationship, (assay NASR)=(ACR), in the same manner as other assay NF values. If it is assumed to be the only assay variable NF which has an effect on the assay NASR, the further the PSSR value deviates from one, the further the NASR deviates from the ACR. Little information is available concerning the prior art incidence of particular gene comparisons associated with PSSR≠1 assay values. The occurrence of particular gene comparison assay values which deviate from one by 1.5-2 fold or so, are plausible, and may occur at a significant frequency.

Note that the above discussion and Table 19 pertain directly to cell sample directly labeled LPN comparisons.

A PSSR, PS-HKR, and PSAR assay value is associated with each DGDS and DGSS particular gene comparison in an assay.

Effect of MLDR on the Relationship (Assay NASR)=(ACR) for a Microarray Gene Comparison of Type 1 LPN.

The prior art normalization process uses assay values for prior art known assay variable NFs to convert the assay RASR value for each particular gene comparison to an assay NASR value for that gene comparison. Prior art defines the assay NASR as the assay measured N-DGER for a particular gene comparison. Further, prior art belief is that the assay measured N-DGER equals the T-DGER, which exists in the compared cell samples for the gene comparison. Prior art does not determine the assay MLDR for a particular gene comparison, and therefore does not take the MLDR value into consideration during the process of normalization of assay observed RASR values. As a consequence, each prior art particular gene comparison assay NASR and N-DGER result which is associated with an MLDR≠1, is inaccurate to the extent that the assay MLDR for the gene comparison deviates from one. The basis for this MLDR effect is discussed below. However, it will first be useful to discuss the characteristics of Type 1 LPN molecule preparations.

The vast majority of prior art microarray gene comparison assays concern the comparison of Type 1 cell sample mRNA LPN molecules. Type 1 mRNA LPNs are usually produced by chemically or enzymatically incorporating label molecules more or less randomly along the length of the LPN molecule. For such Type 1 LPN molecules, the number of label molecules associated with the LPN molecule increases in essentially direct proportion to an increase in LPN molecule nucleotide length. Similarly, the number of label molecules associated with a particular mRNA LPN population which is present in a cell sample mRNA LPN preparation, increases in essentially direct proportion to an increase in the TNC of such a particular mRNA LPN molecule population. A randomly labeled Type 1 LPN molecule population has a TPN equal to one or more. A different kind of Type 1 LPN molecule population is one which has a TPN equal to two or more, and has the same number of label molecules associated with each individual LPN molecule, whether the individual LPN molecule has a short or long nucleotide length. Here, the number of label molecules increases in direct proportion to an increase in TPN for the mRNA LPN population. Type 1 randomly labeled LPN molecules can be characterized by the quantitative label signal activity per microgram of LPN. As discussed earlier the quantitative label signal activity per microgram of such a cell sample total mRNA LPN, is termed the total signal activity of the LPN preparation or the TSA, and the ratio of two compared cell samples TSA values is the TSAR. As also discussed earlier, the quantitative label signal activity per microgram of a particular gene mRNA LPN molecule population which is present in a cell sample LPN preparation, is termed the particular mRNA LPN signal activity or PSA, and the ratio of two compared particular mRNA LPN molecule populations PSA values is termed the PSAR. The TSA value for a cell sample LPN preparation reflects the average label signal activity per microgram for all of the LPN molecules present in the cell sample LPN prep.

The assay MLD value for a particular mRNA LPN molecule population which is present in a gene comparison assay is a measure of the total length of the maximum number of mRNA LPN molecules which can be hybridized by a single ECDP molecule. This is equivalent to stating that the MLD for a particular mRNA LPN molecule population is a measure of the total mass of particular mRNA LPN molecules which can be hybridized by a single CDP molecule.

The effect of the assay MLDR on the relationship, (assay NASR)=(ACR), can be illustrated with an idealized microarray cell sample SGDS gene comparison assay. For this assay, total mRNA is isolated from Cell Sample 1 and Cell Sample 2, and a Type 1 randomly labeled total mRNA LPN preparation is produced for each cell sample. The Cell Sample 1 LPN molecules are labeled with a particular signal molecule, and Cell Sample 2 LPN molecules are labeled with a different type of signal molecule. The signal from each of these different label molecules is readily detected in the presence of the other label molecule. The assay TSAR is equal to one, and the assay PSAR equals one for each particular gene comparison in the assay, including the particular Gene B mRNA LPN comparison. Gene B mRNA has an undegraded nucleotide length of 2000 nucleotides. The microarray assay hybridization solution contains an equal mass of each cell samples total mRNA LPN preparation. In the microarray assay hybridization solution, the ACR=1 for the Gene B mRNA LPN molecule population comparison. An ACR=1 for the Gene B LPN comparison indicates that in the assay hybridization solution the molar concentration of Cell Sample One mRNA LPN molecules which represent Gene B, equals the molar concentration of Cell Sample Two differently labeled mRNA LPN molecules which represent Gene B. The microarray slide used in this idealized assay has only one spot, and that spot contains a Gene B specific CDP with a specified ECDP value. The assay hybridization solution containing each cell sample's differently labeled LPN molecules is placed on the microarray spot, and then the slide is incubated under the appropriate hybridization conditions so that each cell sample's Gene B LPN molecules, can hybridize to the one microarray spot CDP. The kinetics of hybridization of each cell sample's Gene B LPN molecules with the spot immobilized Gene B CDP molecules, is determined by the immobilized CDP molecule concentration. Here, it is assumed that there is no difference in the hybridization kinetics of short or long nucleotide length Gene B LPN molecules with the Gene B CDP. At the end of the hybridization step the ratio of, (the number of Cell Sample 1 Gene B LPN molecules which are specifically hybridized to the spot)÷(the number of Cell Sample 2 Gene B LPN molecules which are specifically hybridized to the spot), will equal the known assay ACR value of one. After the hybridization and posthybridization processing, the spot associated signal activity for each different label is quantitatively measured to obtain the total spot signal (TSS) for each different label. Assay background is then subtracted from each different label's TSS to obtain a raw assay signal (RAS) for each label. The ratio of, (the Sample 1 RAS value)÷(the Sample 2 RAS value), is then the assay RASR value for this idealized Gene B LPN comparison. This RASR value is then normalized for a pertinent non-MLDR assay variable NFs to give an NASR value. It is assumed for this idealized assay that, aside from assay factors which determine the MLDR, there are no other assay variables which affect the relationship (assay NASR)=(ACR). Under such conditions, when the assay MLDR=1, for a particular gene comparison, the (assay NASR)=(ACR) for the gene comparison.

Table 20 illustrates the effect of the assay MLDR on the relationship (observed assay NASR)=(actual ACR for the particular gene comparison which exists in the assay hybridization solution). Table 20 indicates that for a particular gene comparison, the further the MLDR deviates from one, then the further the observed assay NASR value deviates from the gene comparison assay's actual ACR value. The assay MLD is usually described in terms of maximum nucleotide length detectable, because this factor is easier to describe and determine in terms of nucleotide length. However, the MLD value for a particular cell samples mRNA B LPN molecule population, is equivalent to the maximum or total mass of the cell sample's mRNA B LPN molecules, which can be hybridized by a single CDP molecule.

TABLE 20
The Effect of MLDR on the Relationship (Assay NASR) = (ACR) For Gene B
Comparison of Type 1 LPN Molecules

| | | | | | | | | ^{(c)}Relative | Relative | | |
|---|---|---|---|---|---|---|---|---|---|---|---|
| | | | LPN | | | | | Mass of | Signal | | Ratio |
| Compared | | | Nucleotide | TNC of | | | | Gene B | Activity | | Gene B |
| Cell Sample | | Assay | Length in | LPN in | Assay | Assay | Assay | LPN in | Observed | ^{(d)}Assay | (Observed NASR) |
| LPN | | TPN | Assay | Assay | ECDP | MLD | MLDR | Spot | in Spot | NASR | (Actual ACR) |
| (i) | 1 | 1 | 2000 | 2000 | 50 | 2000 | 1 | 1 | 1 | 1 | 1 |
| | 2 | 1 | 2000 | 2000 | 50 | 2000 | | 1 | 1 | | |
| (ii) | 1 | 10 | 200^{(a)} | 2000 | 50 | 200 | 1 | 1 | 1 | 1 | 1 |
| | 2 | 10 | 200^{(a)} | 2000 | 50 | 200 | | 1 | 1 | | |
| (iii) | 1 | 1 | 2000 | 2000 | 50 | 2000 | 10 | 10 | 10 | 10 | 10 |
| | 2 | 1 | 200^{(a)} | 200^{(a)} | 50 | 200 | | 1 | 1 | | |
| (iv) | 1 | 10 | 100^{(a)} | 1000^{(a)} | 1000 | 1000 | 2.5 | 2.5 | 2.5 | 2.5 | 2.5 |
| | 2 | 4 | 100^{(a)} | 400^{(a)} | 1000 | 400 | | 1 | 1 | | |
| (v) | 1 | 1 | 500^{(a)} | ˜500^{(a)} | 400 | 500 | 1 | 1 | 1 | 1 | 1 |
| | 2 | 4 | 500^{(a)} | 2000 | 400 | 500 | | 1 | 1 | | |
| (vi) | 1 | 1 | 2000 | 2000 | 500 | 2000 | 20 | 20 | 20 | 20 | 20 |
| | 2 | 1 | 100^{(a)} | 100^{(a)} | 100^{(b)} | 100 | | 1 | 1 | | |
| (vii) | 1 | 1 | 1200^{(a)} | 1200^{(a)} | 300 | 1200 | 3 | 3 | 3 | 3 | 3 |
| | 2 | 1 | 400^{(a)} | 400^{(a)} | 300 | 400 | | 1 | 1 | | |
| (viii) | 1 | 5 | 400^{(a)} | 2000 | 600 | 800 | 4 | 4 | 4 | 4 | 4 |
| | 2 | 1 | ˜200^{(a)} | ˜200 | 600 | 200 | | 1 | 1 | | |

^{(a)}Less than 2000 nucleotides for TNC and nucleotide length may occur because of mRNAdegradation or labeling procedure, or both.
^{(b)}Under certain conditions, a single CDP can have two assay ECDP values.
^{(c)}Keep in mind that the Gene B mRNA LPN PSAR = 1.
^{(d)}All ratios have the Cell Sample 1 parameters in the numerator.

For this idealized example, it will be useful to discuss the effect of the MLDR on the relationship (assay NASR)=(ACR), by describing the MLD values in terms of the mass of each compared cell sample's mRNA B LPN molecules which hybridizes to the ECDP spot during the assay. This is incorporated into Table 20.

In this idealized assay, the MLDR effect arises from the interaction of two factors. First, during the assay hybridization step, the mass of mRNA B LPN molecules which can hybridize to a single ECDP molecule is greater for one compared cell sample than the other, even though each compared cell sample mRNA B LPN is present at the same molar concentration in the hybridization solution. Second, each compared cell sample mRNA B LPN molecule population has the same quantitative label signal activity per mass of mRNA B LPN. This is illustrated by Table 20 (iii) where: A Cell Sample 1 mRNA B LPN molecule has a nucleotide length and mass which is 10 times greater than that of Cell Sample 2 mRNA B LPN molecules; the TPN for each cell sample mRNA B preparation equals one; each ECDP molecule has a nucleotide length of 50 nucleotides, and each single ECDP molecule can hybridize to only one mRNA B LPN molecule, whether it is short or long. Here, after hybridization, each ECDP molecule hybridized to a long Cell Sample 1 mRNA B LPN molecule, will be associated with ten times greater LPN mass and signal activity, relative to each ECDP molecule which is associated with a short Cell Sample 2 LPN molecule. Because of this, the MLDR=10 for the gene comparison. Table 20 (iv) illustrates another assay situation where: the mRNA B LPN molecules for Cell Sample 1 and 2 have the same 100 nucleotide length, and the same mass; the assay TPN for each cell sample mRNA B LPN is different, and equals ten for Cell Sample 1, and four for Cell Sample 2; each ECDP molecule has a nucleotide length of 1000 nucleotides, and each single ECDP molecule can hybridize to 10 different 100 nucleotide long mRNA B LPN molecules. Here, after hybridization, each ECDP molecule hybridized to Cell Sample 1 mRNA B LPN molecules can be associated with 10 different 100 nucleotide long LPN molecules, while each ECDP molecule hybridized to Cell Sample 2 mRNA B LPN molecules can be associated with only 4 different 100 nucleotide long LPN molecules. Consequently, each ECDP molecule hybridized with Cell Sample 1 mRNA B molecules will be associated with 2.5 times greater LPN mass and signal activity, relative to each ECDP molecule associated with Cell Sample 2 mRNA B LPN. Because of this the MLDR=2.5 for the gene comparison.

The illustrations of Table 20 indicate that for a particular microarray assay Gene B comparison, when the assay MLDR=1, then the relationship (assay NASR)=(ACR) is valid, and that when the assay MLDR≠1, then the relationship is not valid. Further, Table 20 indicates that when the MLDR≠1, then the magnitude of the deviation of the MLDR from one, is equal to the magnitude of the deviation of the observed RASR value from the actual ACR value for the Gene B comparison. Clearly the assay MLDR value must be taken into consideration, and when the assay MLDR≠1 the idealized assay and prior art assay observed NASR values must be normalized for the bias introduced by the MLDR assay variable.

As discussed, prior art practice does not determine assay MLDR values, and therefore does not take into consideration the assay MLDR values during the prior art normalization process. Further, it is not reasonable to believe that the assay MLDR is always equal to one for each particular SGDS, Type 1 LPN gene comparison in an assay. Each Table 20 example which illustrates results for an assay MLDR≠1, represents a plausible microarray gene comparison assay scenario which can occur in reality because of imperfections in the assay procedures, processes, and materials. These examples include only a few of the possible situations where MLDR≠1.

Table 20 (i) represents an assay situation where both compared cell sample isolated mRNA's are undegraded, and an oligo dT primer labeling method produces full sized LPN molecules. Current knowledge indicates that such a situation rarely occurs in prior art microarray practice, since LPN molecules produced from undegraded mRNA's are generally shorter in nucleotide length than the undegraded mRNA molecules used to produce them.

Table 20 (ii) is consistent with an assay where both cell sample isolated mRNA's are undegraded, and random priming is used to produce the compared LPNs. Table 20 (ii) is also consistent with an assay situation where both cell sample isolated mRNA's are degraded to about the same extent and random primers are used to produce the compared LPN's, or the compared LPN's are produced by direct chemical labeling of the mRNAs.

Table 20 (iii) is consistent with an assay situation where: Cell Sample 1 isolated mRNA is undegraded, and oligo dT primer is used to produce the Cell Sample 1 LPN; while Cell Sample 2 isolated mRNA is considerable degraded, and oligo dT primer is used to produce the short cell Sample 2 LPN molecules, which also have a low TNC, and which represent the 3′ end of the Cell Sample 2 mRNA molecules. Alternatively Table 20 (iii) is consistent with an assay situation where the Cell Sample 2 isolated mRNA is undegraded but impure, and the impurity results in uniform early termination of LPN molecule synthesis during the oligo dT primer mediated production of the Cell Sample 2 LPN, thereby resulting in short Cell Sample 2 LPN molecules which also have a low TNC. Table 20 (i), (ii), and (iii) have ECDP values of 50 nucleotides and represent oligonucleotide microarrays.

Table 20 (iv) is consistent with an assay situation where both the Cell Sample 1 and 2 isolated mRNA's are degraded before isolation, but to different extents. As a consequence, the TNC and TPN of the Cell Sample 1 LPN molecules produced with random priming, are different from the TNC and TPN of the Cell Sample 2 LPN molecules also produced by random priming. Here, although the nucleotide length of each compared LPN molecules is the same, the MLDR≠1.

Table 20 (v) is consistent with an assay situation where: the Cell Sample 1 mRNA is degraded before isolating the Poly A mRNA fraction, and random primers are used to produce the LPN, which represents the 3′ end of the Cell Sample 1 mRNA molecules; while Cell Sample 2 isolated mRNA is undegraded and random primers are used to produce the Cell Sample 2 mRNA molecules, which represent the entire length of the Cell Sample 2 mRNAs.

Table 20 (vii) is consistent with an assay situation where, both cell samples isolated mRNAs are degraded and Cell Sample 1 mRNA is less degraded, and oligo dT priming is used to produce both cell samples LPNs. Table 20 (iv)-(viii) represent cDNA microarray assays.

As discussed, prior art normalizes to convert the assay RASR for a gene comparison to the NASR for the gene comparison. Prior art defines the NASR as representing the assay measured N-DGER for the gene comparison, and believes that the N-DGER is equal to the T-DGER for the gene comparison. It is clear that when the assay MLDR value is equal to one, it does not influence the assay NASR value, and that when the assay MLDR value is not equal to one, the assay NASR is influenced. Further, it is not reasonable to believe that all prior art microarray gene comparisons have an assay MLDR equal to one, but it is reasonable to believe that the assay MLDR value for many prior art gene comparisons is not equal to one. Prior art practice does not determine the assay MLDR value for microarray gene comparisons, and therefore does not take the MLDR into consideration, during the process of determining the assay NASR and N-DGER for a gene comparison. In this situation, absent some knowledge of the assay MLDR value for each microarray assay SGDS Type 1 LPN gene comparison, it cannot be known whether the assay NASR and N-DGER value for a particular gene LPN comparison accurately reflects the gene comparison's ACR value. In other words, absent some knowledge of the assay MLDR for a particular gene comparison, it cannot be known whether any particular prior art gene comparison NASR or N-DGER result value contains assay bias due to the MLDR effect. This adds to the difficulty in interpreting prior art NASR and N-DGER results caused by prior art's absence of knowledge concerning the assay SCR value, which was discussed earlier.

In the context of the above discussion, the prior art belief that for a particular gene comparison the prior art normalized NASR and N-DGER value is always equal to the ACR for the particular gene comparison, is not valid.

Effect of MLDR on the Relationship (Assay NASR)=(ACR) for a Microarray Gene Comparison with Type 2 LPN.

Very few prior art microarray gene comparison assays use Type 2 LPN molecules. As discussed earlier, for a cell sample total mRNA Type 2 LPN preparation, each particular mRNA LPN molecule population present must have a TPN equal to one, or nearly one, and each particular mRNA LPN molecule in the LPN preparation must have the same, or nearly the same LLN and LLS value, whether it is short or long in nucleotide length. Thus, the label signal activity associated with each individual Type 2 LPN molecule, does not increase or decrease with the length of the LPN molecule or the mass of the LPN molecule.

The effect of the MLDR on the relationship (assay NASR)=(ACR), when Type 2 LPNs are compared can be illustrated by using a modified version of the idealized SGDS microarray Gene B comparison assay described in the previous section. For this use of the idealized assay, cell sample mRNA B Type 2 LPN molecules will be compared, and it will be assumed that the assay LLNR=1. Note that here, as before, it is assumed that there is no difference in the hybridization kinetics of short or long nucleotide length LPN's with the CDP, and that the assay label signal activity per label molecule is the same for each different label.

Table 21A & B (together representing one table) illustrate the effect of MLDR≠1 on the relationship (assay NASR)=(ACR), when Type 2 cell sample mRNA B LPN molecules are compared in the idealized assay. Table 21A & B clearly illustrate that MLDR ≠1 assay values have no effect on the said relationship. This occurs because during the hybridization step; the number of Cell Sample 1 mRNA B LPN molecules which hybridizes to the CDP spot is the same as the number of Cell Sample 2 mRNA B LPN molecules which hybridize to the spot; and each Cell Sample 1 and Cell Sample 2 mRNA B LPN molecule which has hybridized, is associated with the same number of label molecules; and the Cell Sample 1 and Cell Sample 2 different label molecules each have the same quantitative signal activity per label molecule.

TABLE 21A
The Effect of MLDR on the Relationship (Assay NASR) = (ACR) For
Gene B Comparison of Type 2 LPN Molecules

| Compared | | | LPN | | | | |
|---|---|---|---|---|---|---|---|
| Cell | | | Nucleotide | TNC of | | | |
| Sample | | Assay | Length In | LPN In | Assay | Assay | Assay |
| LPN | | TPN | Assay | Assay | ECDP | MLD | MLDR^{(c)} |
| (i) | 1 | 1 | 2000 | 2000 | 50 | 2000 | 1 |
| | 2 | 1 | 2000 | 2000 | 50 | 2000 | |
| (ii) | 1 | 1 | 2000 | 2000 | 50 | 2000 | 10 |
| | 2 | 1 | 200^{(a)} | 200^{(a)} | 50 | 2000 | |
| (iii) | 1 | 1 | 300^{(a)} | 300^{(a)} | 1000 | 300 | 0.25 |
| | 2 | 1 | 1200^{(a)} | 1200^{(a)} | 1000 | 1200 | |
| (iv) | 1 | 1 | 1200^{(a)} | 1200^{(a)} | 300 | 1200 | 3 |
| | 2 | 1 | 400^{(a)} | 400^{(a)} | 300 | 400 | |

TABLE 21B
The Effect of MLDR on the Relationship (Assay NASR) = (ACR) For Gene B
Comparison of Type 2 LPN Molecules

| | | Relative | Relative | | | |
|---|---|---|---|---|---|---|
| | | Mass of | Number of | Relative Signal | | Ratio |
| Compared Cell | | Gene B | Gene B LPN | Activity | | ^{(b)}Gene B |
| Sample | | LPN in | Molecules In | Observed | Assay | (Observed NASR) |
| LPN | | Spot | Spot | In Spot | NASR | (Actual ACR) |
| (i) | 1 | 1 | 1 | 1 | 1 | 1 |
| | 2 | 1 | 1 | 1 | | |
| (ii) | 1 | 10 | 1 | 1 | 1 | 1 |
| | 2 | 1 | 1 | 1 | | |
| (iii) | 1 | 1 | 1 | 1 | 1 | 1 |
| | 2 | 4 | 1 | 1 | | |
| (iv) | 1 | 3 | 1 | 1 | 1 | 1 |
| | 2 | 1 | 1 | 1 | | |

^{(a)}Less than 2000 nucleotides for TNC and nucleotide length may occur because of RNA degradation or the labeling procedure.
^{(b)}Here the ACR = 1.
^{(c)}All ratios have Sample 1 in the numerator.

Thus, for microarray SGDS, DGDS, and DGSS gene comparison assays which compare Type 2 LPN molecules, the MLDR has no effect on the assay NASR values for particular gene comparisons. This allows the design of microarray gene expression comparison assays where the MLDR non-global assay variable NF is effectively equal to one and can be ignored.

Note that the above-described discussion and tables apply directly to cell sample comparisons of directly labeled LPN preps. In addition, for cell sample comparisons of indirectly labeled particular gene L-LPNs, the MLDR assay value can be used to help determine the particular gene comparison SBNR value.

Effect of Assay Hybridization Kinetic Factors on the Relationship (Assay NASR)=(ACR) for Microarray Comparisons of Type 1 and 2 LPN Molecules.

During the normalization process of converting assay RASR values to assay NASR values, the prior art does not take into consideration effects on the assay hybridization kinetics of the mRNA LPN molecules with the assay ECDP, which are associated with nucleotide length or nucleotide sequence, nucleotide composition, or LD effect differences between the compared particular mRNA LPN molecules. As discussed earlier such differences, if large enough, can have an effect on the LPN hybridization kinetics in the assay. Generally, when in solution long nucleotide length LPN molecules will hybridize faster than short nucleotide length LPN molecules, and LPN molecules with weak nucleotide sequence related intramolecular secondary structure, will hybridize faster than LPN molecules with very strong secondary structure. This applies to both Type 1 and Type 2 LPN molecules. It has been reported that for the hybridization of LPN molecules in solution to surface immobilized CDP, the rate of hybridization is inversely proportional to the nucleotide length of the LPN and that shorter LPNs hybridize significantly faster than longer LPN molecules.

Differences in the hybridization kinetics of compared particular RNA transcript LPN molecules can affect the relationship (NASR)=(ACR). This occurs because the particular LPN molecules, which hybridize fastest to the CDP will, relative to their actual proportion in the assay hybridization solution, overcontribute to the assay signal value. As discussed earlier, the hybridization kinetics assay variable NF associated with any nucleotide length differences is termed the PL-HKR, while the hybridization kinetics assay variable NF associated with nucleotide sequence is termed the PS-HKR. Both the PL-HKR and the PS-HKR are non-global assay variable NFs.

Prior art microarray normalization practice does not take either the PL-HKR or PS-HKR into consideration, and seldom determines the nucleotide lengths of the compared LPN molecules. For microarray gene comparison assays, prior art presumably assumes that since the compared LPN molecules are produced from the same mRNA, significant nucleotide sequence differences will not be present. This may not be the case for gene comparison assays which compare mRNA LPN molecules of significantly different nucleotide length. It appears that prior art practice also assumes that differences in the compared LPN molecules nucleotide length do not cause hybridization kinetic differences.

The effect of the PL-HKR or PS-HKR on the relationship (assay NASR)=(ACR), can be illustrated by using a modified version of the idealized microarray Gene B comparison assay described for Table 20. For this use of the idealized assay: Cell Sample 1 or 2 Type 1 or Type 2 mRNA LPNs will be compared; it will be assumed that the only assay variable NF which can affect the NASR is the PL-HKR or the PS-HKR.

TABLE 22
The Effect of PL-HKR or PS-HKR on the Relationship (Assay NASR) = (ACR) For
Type 1 or Type 2 LPN Gene B Comparisons

| | | Gene B LPN | Gene B | | | |
|---|---|---|---|---|---|---|
| Compared | ^{(a)}Gene B | Relative Assay | Assay PL- | Observed | Observed | Ratio of |
| Cell Sample | ACR of | Hybridization | HKR or | Assay | Assay | (Assay NASR) |
| LPN | Assay | Kinetics | PS-HKR | RAS | NASR | (Assay ACR) |
| 1 | 1 | 1.5 (fast) | 1.5 | 1.5 | 1.5 | 1.5 |
| 2 | | 1 (slow) | | 1 | | 1 |
| 1 | 1 | 4 | 4 | 4 | 4 | 4 |
| 2 | | 1 | | 1 | | 1 |
| 1 | 1 | 1 | 0.5 | 0.5 | 0.5 | 0.5 |
| 2 | | 2 | | 1 | | 1 |
| 1 | 1 | 1 | 1 | 1 | 1 | 1 |
| 2 | | 1 | | 1 | | 1 |

^{(a)}All ratios have Cell Sample 1 parameters in numerator.

Table 22 illustrates the effect of the PL-HKR or PS-HKR on the relationship (assay NASR)=(ACR). Table 22 indicates that the for a particular gene comparison, the further the assay PL-HKR or PS-HKR deviates from one, the further the assay NASR deviates from the particular gene comparisons ACR which is present in the microarray assay hybridization solution.

Note that the above discussion and Tables apply directly to both cell sample comparisons of directly labeled LPNs or indirectly labeled L-LPNs. Note further that PL-HKR and PS-HKR assay values are also associated with each DGDS and DGSS particular gene comparison in an assay.

Effect of PCR Amplification Efficiency (E) or AE•AE Values on the Relationship (NASR)=(ACR) for an RT-PCR Assay.

For prior art RT-PCR assays the early described third tacit assumption concerns the PCR amplification efficiency (E) or AE•AE values associated with particular gene comparisons and particular gene and standard comparisons. The E and AE•AE terms were defined earlier, and are closely related. For simplicity, this discussion will emphasize the E term. The E and AE•AE values associated with an RT-PCR assay can affect the validity of the relationship (NASR)=(ACR) for particular gene comparisons, and particular gene and standard comparisons, and standard comparisons. The third tacit assumption also concerns the assay associated particular gene and standard AE•SE values. Since these AE•SE values do not affect the validity of the relationship (NASR)=(ACR) and have been discussed earlier, they will not be discussed here. For this discussion it will be assumed that the AE•SE values for assay compared particular genes, compared particular genes and standards, and compared standards, are the same, and that the only assay factor which can cause the assay measured NASR to deviate from the ACR is the validity of the E or AE•AE aspect of the third tacit assumption.

Most prior art RT-PCR assays practice the third tacit assumption and assume that the assay E or AE•AE values which are associated with particular gene comparisons, particular gene and standard comparisons, and standard comparisons, are essentially the same, or are equal to one (117, 118). Other prior art RT-PCR assays determine one or more E values for particular gene and standards in a reference system, and then assume that these values can be validly used during the assay result normalization process. These other prior art assays also make assumptions about the assay E values. These assumptions will be discussed below.

The variability of the RT-PCR assay associated E values is a prior art considered assay variable, and prior art does, at times, consider such E values during the assay result normalization process. However, the prior art RT-PCR assay associated normalization process for assay E value differences cannot be known to be valid, and in many cases is almost certainly invalid. This is discussed below.

For prior art RT-PCR assays it is known that the cDNA ALGAE assay values for cell sample, particular gene, and standard AE cDNA preps are virtually always equal to significantly less than one. Prior art reported particular gene and standard E values generally range from 0.7 to 0.95, and are often lower than 0.7. For a thirty cycle PCR assay this translates into a generally occurring ALGAE assay value range which varies from 0.008 to 0.21, a difference of about 25 fold. It is well known that for an RT-PCR assay, the particular gene and standard E and AE•AE assay values can be affected by a large variety of commonly occurring factors which can cause the E values for, different particular gene cDNA AEs in the same RT-PCR assay tube, or for particular gene and standard cDNA AEs in the same assay tube, or for different standard cDNA AEs in the same assay tube, to be significantly different. These factors include but are not limited to the following. Differences in the design characteristics of particular gene and/or standard PCR primers, including differences in the nucleotide length and/or nucleotide sequence and/or nucleotide composition of the particular gene and/or standard PCR primers. Also differences in the characteristics of the particular gene and/or standard amplicon equivalents to be amplified, including differences in the concentration of amplicons being amplified, differences in the nucleotide length and/or nucleotide sequence and/or nucleotide composition of the particular gene and/or standard cDNA molecules and cDNA amplicon molecules. Complicating the situation, even in the same RT-PCR assay amplification solution the E value for a particular gene or standard amplicon amplification decreases over the course of the amplification reaction, and it is possible that differences in the rate of decrease occur for different particular gene and/or standard cDNA AE or DNA AE molecules during the course of the PCR amplification reaction. The above-described same RT-PCR assay solution factors would affect the biological accuracy of the assay measured particular gene RNA transcript RN value and the particular gene N-DGER value derived from it, and the validity of the relationship (NASR)=(ACR), which is associated with the particular gene comparison N-DGER value.

It is also well known that particular gene and standard E and AE•AE values can be affected by a variety of commonly occurring factors which can cause the E values for, the same or different particular gene cDNA AEs in different RT-PCR assay tubes, or for different particular gene and standard cDNA AEs in different RT-PCR tubes, or for the same standard cDNA AEs in different RT-PCR tubes, to be significantly different. These factors include, but are not limited to the following. Differences in the particular gene and/or standard amplicon concentrations in the amplification solution, as well as differences in, the amplification solutions, the amplification temperatures, the amplification times for different aspects of an amplification cycle, the rates of accumulation of reaction byproducts, the rates of inactivation of the DNA polymerase, and the rates of decrease of the E values during the amplication reaction. In addition, differences in compared cell sample RNA purities and/or differences in compared cell sample particular gene mRNA transcript nucleotide lengths and/or differences in compared cell sample particular gene cDNA and standard cDNA nucleotide lengths and/or cDNA prep purity. The above-described different RT-PCR between assay factors would affect the biological accuracy of the comparative assay measured particular gene N-DGER value, and the validity of the relationship (NASR)=(ACR), which is associated with the particular gene comparison N-DGER value.

Prior art RT-PCR and PCR assay practice often assumes that the within assay solution assay AE•AE values for an assay are the same, or equal to one. As discussed, it is not uncommon for different particular gene cDNA AEs, or different standard cDNA AEs, or particular gene and standard cDNA AEs, in the same assay solution to have significantly different E or AE•AE values. Prior art RT-PCR practice also often assumes that the between assay E values for, the same particular gene cDNA AEs are the same, a particular gene and standard cDNA AEs are the same, and the same standard cDNA AEs, are the same. As discussed, it is not uncommon for the same particular gene cDNA AE associated E values, as well as the same standard cDNA AE associated E values, to be significantly different in separate assays.

Many prior art gene expression analysis RT-PCR assays do not determine the assay E or AE•AE values for either the particular gene of interest or standards which are associated with the assay, while some do. In general prior art RT-PCR practice takes a casual view of these assay E values and their importance for accurate quantitation, and does not often take such E values into consideration when determining and interpreting particular gene measured RN or particular gene comparison N-DGER values. The determination of the particular gene or standard E values which are associated with prior art RT-PCR assay gene expression analyzes for particular genes in an unknown cell sample, are almost always done by determining a statistically significant value for the particular gene or standard E value in a reference system. Prior art then assumes that the reference system determined particular gene or standard E value is valid for the accurate determination of the particular gene RN values and particular gene comparison N-DGER values by RT-PCR assays which analyze unknown cell samples. For RT-PCR assays which do not utilize a standard, this approach assumes that for the determination of biologically accurate unknown cell sample particular gene RN values, the particular gene E value must equal one in the reference system and the unknown cell sample assay. Prior art also assumes that for the determination of biologically accurate unknown cell sample comparison particular gene N-DGER values, the compared unknown cell sample particular gene E values must be the same, or equal the reference system particular gene E value. As an example of this approach, Applied Biosystems, Inc., which is the leading company with regard to the use of pre-designed RT-PCR assays for quantitative particular gene expression analyzes for unknown cell samples, pre-determines a particular gene assay E value in a reference system. ABI indicates that it is not necessary to measure the E value for an ABI unknown cell sample particular gene RT-PCR assay. ABI states that the particular gene E value has been pre-determined to have a statistically significant average assay value of close to one in an ABI RT-PCR assay reference system which is free of PCR inhibitors, and ABI claims that the E value for the ABI unknown cell sample particular gene RT-PCR assay does not need to be measured, because it will also be equal to close to one. ABI represents that an ABI RT-PCR assay particular gene E value is equal to close to one when measured with the best method available. More specifically the ABI claimed particular gene E value equals 1±10% for the ABI RT-PCR assay, which is free of PCR inhibitors. This means that for the PCR inhibitor free system the particular gene E values for the ABI RT-PCR assay replicates varied from 0.9 to 1.1. ABI further represents that all of their TaqMan gene expression RT-PCR particular gene expression assays are associated with particular gene E values which are equal to 1±10%, and because of this all of their TaqMan gene expression assays are equivalent. ABI does not discuss how it is possible to obtain an assay E value of greater than one. ABI acknowledges that even in the PCR inhibitor free reference assay system, different replicate assays for one particular gene have assay E values which differ by ±10% from one, and also that E values which are associated with ABI assays for different particular genes also differ by ±10% from one. ABI did not provide information on the particular gene E values, which are associated with different unknown cell samples, and does not recommend the determination of the E value for each unknown cell sample assay. It seems very likely that such differences in E values between replicates of one particular gene assay, and between different assays for different particular genes will be greater in unknown cell sample assays where PCR inhibitors are commonly present. The source of this information (117) is the ABI application note titled “Amplification Efficiency of TaqMan Gene Expression Assays,” which was obtained from the ABI web site (www.appliedbiosystems.com), in late 2004.

The above discussion of prior art RT-PCR assays, which do not use standards, indicates the following. For the determination of particular gene RN values it is unlikely that, and it cannot be known that, the particular gene assay E value is equal to one, and therefore the third tacit assumption cannot be known to be valid, and is very likely to be invalid, for these RT-PCR assays. Further, for the determination of particular gene comparison N-DGER values, it is unlikely that, and it cannot be known that, the compared particular gene assay E values are the same, and therefore the third tacit assumption cannot be known to be valid, and is likely to be invalid for these RT-PCR assays.

ABI indicates that for a particular gene TaqMan analysis, different replicate reference system assays which are done in the absence of PCR inhibitors, are associated with particular gene E values which differ by as much as 1±10%. This means that for an SGDS cell sample particular gene comparison the compared particular gene E values can differ by as much as about 20%, or in other words vary from E=1.9 for one cell sample to E=1.1 for the other cell sample. From the ABI data which is presented in the applications note, it appears that even for a large number of replicates assay E values for a particular gene, a one standard deviation value of ±5 percent at a minimum, is associated with the set of E values presented in the document. This one standard deviation value of ±5 percent indicates that even in the absence of PCR inhibitors in the assay sample, about one in three replicate assay E values is likely to deviate by greater than ±5 percent. As discussed earlier it is known that the presence of PCR inhibitors is common in unknown cell samples. This makes it likely that for the ABI unknown cell sample assay, the magnitude of the standard deviation associated with the same particular gene E value, is significantly larger than for the reference system assays. It is reasonable to believe that one standard deviation values of ±8 percent or greater are not uncommon for such E values in unknown cell sample assays. In order to determine an unknown cell sample particular gene RN value, or an unknown cell sample comparison N-DGER value, the ABI system also employs one or more exogenous and/or endogenous standards for the assay. Prior art standard methods involve one of the following situations. (a) The co-amplification of the standard and particular gene cDNA AEs which are present in the unknown cell sample cDNA AE prep, in a single PCR step tube. (b) The separate amplification of standard or particular gene cDNA AEs which are present in the same unknown cell sample cDNA AE prep, in separate PCR tubes. (c) The separate amplification of a standard cDNA AE which is present in a reference system cell sample cDNA prep, and a particular gene cDNA AE which is present in an unknown cell sample cDNA AE prep, where the standard is an exogenous standard mRNA transcript. (d) As c where the standard is mRNA transcript for the particular gene of interest. (e) As c where an endogenous standard and the particular gene cDNA AEs which are present in the unknown cell sample cDNA AE prep are co-amplified together in the same tube. For each of the situations a-d, it is assumed that the compared unknown cell sample cDNA AE•SE values are the same, and that compared standard and particular gene AE•SE values are the same. For all of these situations, in order to obtain accurate prior art ABI assay measured particular gene RN and particular gene comparison N-DGER values, the compared particular gene, and compared standard, and compared particular gene and standard, assay E values must be the same. However, as earlier discussed, the assay E values for each different particular gene or standard can often be significantly different when measured in separate PCR tubes, or the same PCR tube, under reference system assay conditions or unknown cell sample assay conditions. Also, the assay E values for a standard can often be significantly different when measured under reference system assay conditions, relative to unknown cell sample assay conditions. In addition, the assay E value for a particular gene can often be significantly different when measured under reference system assay conditions relative to unknown cell sample assay conditions. Further, the assay E value for a particular gene which is measured under unknown cell sample assay conditions, can often be significantly different than the assay E value for a standard measured under unknown cell sample assay conditions in the same or separate PCR tube, even when the particular gene and standard average E values are the same when measured in the reference system assay. All of this indicates that it cannot be assumed that the compared particular gene, compared standard, and compared particular gene and standard, assay E values for the prior art ABI TaqMan assays, as well as other prior art RT-PCR assays, are the same for an unknown cell sample assay. The ABI results indicate that for these assays it is reasonable to believe that the said assay associated compared E values often differ by ±8 percent or more. These ABI results very likely reflect examples of the best prior art practice of the RT-PCR quantitative gene expression practice, and therefore it is likely that the ±8 percent represents a low value for the prior art in general. The effect of such a ±8% value and even smaller values on the deviation from biological accuracy of the prior art RT-PCR measured particular gene RN values and particular gene comparison N-DGER values is discussed later.

Prior art RT-PCR practice sometimes determines the assay particular gene and/or standard E values associated with multiple different unknown cell samples of interest. These multiple assay measured E values for the particular gene and standard cDNA AEs are then processed to obtain the average E value and its standard deviation for the particular gene and standard in the replicate set (191). Prior art then assumes that this value represents the assay particular gene and/or standard E values which are associated with any RT-PCR assay of the unknown cell sample type. This approach acknowledges that particular gene and/or standard E values commonly vary in different unknown sample assays, and attempts to compensate for the variations. The standard deviation for each of these values can then be used to estimate the accuracy limits of the unknown assay measured particular gene RN value or particular gene comparison N-DGER values. The effectiveness of this approach for determining biologically accurate RN and N-DGER values for a particular gene in an unknown sample by RT-PCR depends on the accuracy of the assay measured E values and the magnitude of the standard deviations associated with these average E values. Prior art RT-PCR and PCR assay practice uses this latter approach because it is not practical to determine the PCR E values for each and every different cell sample because the determination of the E values is complex and labor intensive.

Prior art believes and practices that because of the known variability which is associated with the particular gene and/or standard E values, as well as the AE•SE values, it is necessary to use standards in an assay in order to control for these variables and obtain accurate assay results. Because in an assay the particular gene and standard E and AE•AE values are often affected differently, the presence of the standard in the assay can result in even larger deviations from result accuracy, than if the standard is not used.

At present there is no practical and accurate method for controlling and normalizing RT-PCR assay determined particular gene RN values and particular gene comparison N-DGER values for the within, and between, assay deviations of particular gene and/or standard E and AE•AE values, even with the use of standards. Indeed, because of the nature of the problem, the use of standards may be counterproductive. This situation is caused by the many common assay factors which cause the E to deviate from one, and the fact that even very small differences in the E values of compared particular gene cDNA AEs, or compared particular gene and standard cDNA AEs, which may not be practically measurable for an assay, can cause the assay measured particular gene RN or N-DGER values to deviate very significantly from biological accuracy. For a thirty cycle prior art RT-PCR assay, a difference of even five percent between the E values of particular gene cDNA preps compared in separate assays, will cause the assay measured particular gene N-DGER value to deviate from biological accuracy by about twofold. For a single thirty cycle prior art RT-PCR assay, a difference of even five percent between the E values of a particular gene cDNA AE prep and a standard cDNA AE prep in the same assay, will cause the assay measured particular gene RN value to deviate from biological accuracy by about twofold. The deviation of an RT-PCR assay measured particular gene N-DGER value from two such separately derived particular gene RN values, where in each separate assay the particular gene and standard E values differ by five percent, will cause either a fourfold deviation, or no deviation, of the assay measured particular gene N-DGER value from biological accuracy. A fourfold deviation will occur in an assay where for one compared cDNA AE prep assay the particular gene cDNA AE associated E value is five percent larger than the standard associated E value, and for the other compared cDNA AE prep the particular gene cDNA AE associated E value is five percent smaller than the standard associated E value. No deviation will occur when for both compared particular gene cDNA AE preps, the particular gene E value is either five percent larger or smaller than the standard gene associated E value.

Prior art RT-PCR practice routinely claims a measurement accuracy for the RT-PCR assay of ±1.2 to ±2 fold. In this context, for a 30 cycle prior art RT-PCR assay, a 2 to 4 fold deviation of the assay measured RN or N-DGER result from biological accuracy is a very significant deviation. Further, in this same context, for assay measured particular gene RN and N-DGER values a deviation from biological accuracy of 1.4 and 2 fold can occur for a 30 cycle RT-PCR assay when the particular gene E value is 2.5 percent larger than the standard E value. Such deviations from biological accuracy are significant relative to the prior art claimed assay measurement accuracy.

Prior art believes and practices that prior art RT-PCR assay measured particular gene RN values are biologically accurate. Prior art RT-PCR assay practice commonly claims that a particular gene RN value or standard RN value can be measured to an assay measurement accuracy of ±1.2 fold to ±2 fold. Prior art RT-PCR assay practice then believes that the prior art RT-PCR assay measured particular gene RN values are biologically accurate to within ±1.2 fold to ±2 fold.

When the measurement accuracy is ±1.2 fold this indicates that the biologically accurate assay value lies somewhere between, (the measured value×1.2) and (the measured value÷1.2). Similarly, when the measurement accuracy is ±2 fold, then the biologically accurate value lies between, (the measured value×2) and (the measured value÷2). Here, for duplicate assay measurements of a particular gene or standard N-DGER value from the comparison of different cell samples, when the measurement accuracy of the compared RN values is within ±1.2 fold, the measured N-DGER value may deviate from biological accuracy by as much as 1.44 fold. This occurs when the measured RN value for one cell sample is 1.2 fold greater than the biologically accurate value, and the measured RN value for the other compared cell sample RN value is 1.2 fold less than the biologically accurate value. For the ±2 fold assay measurement situation, the measured N-DGER value may deviate by as much as 4 fold from biological accuracy. For a situation where each compared RN value deviates from biological accuracy to the same extent and in the same direction, the derived N-DGER value is biologically correct.

The measurement accuracy value for a particular gene in a prior art RT-PCR assay is usually determined in a well defined reference system by doing replicate determinations in order to obtain a statistically significant value for the assay measurement accuracy, which consists of a mean value and an associated statistic which indicates the probable deviation of the reference system measured mean value from the true value. Prior art then assumes that the reference system measured value and statistic for the assay measurement accuracy is valid for assays involving the quantitation of the particular gene expression in unknown cell samples. Note that an RT-PCR assay measured particular gene RN or N-DGER assay value is always “assay accurate.” That is accurate to within the assay measurement accuracy limits. However, the measured RN or N-DGER values may not be “biologically accurate.” That is the biologically accurate RN or N-DGER values does not lie within the measured RN or N-DGER value assay measurement accuracy limits.

The effect of small unknown cell sample induced changes in the reference system determined particular gene E values on the biological accuracy of the unknown cell sample assay measured values for the particular gene RN and particular gene comparison N-DGER values, is illustrated below. For simplicity of discussion, the following will be assumed. (i) The standard is an exogenous mRNA transcript. (ii) For the reference system, known amounts of standard and particular gene mRNA transcript molecules are added to the reference system cell sample RNA prep which is put into the reference system assay RT step. (iii) The standard and particular gene cDNA AEs are produced and the AE•SE values for the particular gene and standard cDNAs are the same. The cDNA AEs are put into the reference system PCR amplification step and amplified. (iv) The standard and particular gene PCR E values are determined to be the same in the reference system assay and equal to 0.9. (v) In the reference system RT-PCR assays and the unknown cell sample RT-PCR assays the measured particular gene RN values are biologically accurate to within ±1.2 fold. (vi) All unknown cell samples have the same amount of total RNA (T-RNA) per cell, and the same amount of unknown cell sample T-RNA is used in the RT step of each unknown cell sample assay. (vii) The same known number of standard mRNA transcripts are added to each unknown cell sample assay RT step, and the abundance of the standard mRNA transcript in the unknown cell sample T-RNA is known to be equal to one copy per cell. (viii) The particular gene mRNA transcript abundance value in the unknown cell sample T-RNA is known to be one copy per cell. (ix) For all unknown cell sample assay RT steps the AE•SE values for the particular gene and standard cDNA AE preps are the same. (x) The entirety of the cell sample particular gene and standard cDNA AEs are added to the assay PCR step and amplified for 30 PCR cycles. (xi) Here, and in the prior art, the particular gene and standard assay E or AE•AE values are not determined for an unknown cell sample assay. (xii) Here it is not assumed, as would the prior art, that the relationship between the quantitative assay E values in an unknown cell sample assay is the same as in the reference system assay. In other words, it is not assumed here that the particular gene and standard assay E values in each unknown cell sample assay are the same or are known. (xiii) A quantitative measure of the amount of particular gene and standard amplicon DNA which is produced in the assay is determined. (xiv) At this point prior art assumes that the compared particular gene and standard E and AE•AE values are the same, and then uses the measured amount of standard amplicon DNA produced in the unknown cell sample assay, and the known amount of standard mRNA transcript present in the unknown cell sample RT step of the assay, and the measured amount of unknown cell sample assay produced particular gene amplicon DNA, in order to determine the biologically correct amount of particular gene mRNA transcripts which are present in the amount of unknown cell sample T-RNA which was used in the assay RT step. This can be done using the relationship, (number of particular gene mRNA transcript present in the unknown cell sample T-RNA present in the assay RT step)=(number of particular gene amplicons produced in the assay PCR step)×(number of standard mRNA transcripts present in the assay RT step÷number of standard amplicons produced in the assay PCR step). Stated differently, (PG RN)=(PG AN)×(S RN÷S AN) where PG and S represent particular gene and standard, RN is the earlier defined mRNA transcript number, and AN is the newly defined amplicon number value. The mRNA transcript number, or mTN, designates the number of particular RNA transcript molecules which are present in the cell sample RNA put into the assay reverse transcriptase reaction. RN and mTN are used interchangeably herein. The PG AN value is the number of particular gene amplicon molecules produced in the assay PCR step, while the S AN value represents the number of S amplicon molecules produced in the assay PCR step. (xv) The relationship (PG RN)=(PG AN)×(S RN÷S AN) is valid only when the prior art assumption that the particular gene and standard AE•SE, E, and AE•AE values are the same. Here it is assumed that the AE•SE values are the same, and it is not assumed that the E and AE•AE values are the same. In this situation (PG RN)=(PG AN)×(S RN÷S AN)÷(PG AE•AE÷S AE•AE). (xvi) Unknown cell sample particular gene comparison N-DGER values are derived by comparing the unknown cell sample assay measured particular gene RN values measured for different unknown cell samples. (xvii) Here it is assumed that only an unknown cell sample assay associated differential change in a particular gene and/or standard E value which causes the assay ratio of the (particular gene E value)÷(the standard E value) to deviate from one, can affect the biological accuracy of the assay measured unknown cell sample particular gene RN values and the unknown cell sample comparison particular gene N-DGER values.

Following is a discussion of the effect of small, and very small essentially undetectable, differential changes in the particular gene and/or standard assay values for E which are likely to occur in unknown cell sample RT-PCR assay designed to quantitate the expression extent of a particular gene mRNA transcript in a cell sample analysis, or a cell sample comparison analysis. Such changes would have occurred unknown to the prior art. However, even if the prior art was aware that such changes had occurred in the unknown cell sample assay, it would be impractical to determine such differences for each unknown cell sample assay, even if it were possible to experimentally measure such differences.

When the particular gene and standard assay associated E values are the same for individual unknown cell sample RT-PCR analyzes, and for RT-PCR assay comparisons of unknown cell samples, the assay measured particular gene mRNA transcript RN values for each analyzed cell sample, and the assay measured particular gene mRNA transcript comparison N-DGER values, are biologically accurate to within the assay measurement accuracy of ±1.2 fold for the particular gene RN values, and ±1.44 fold for the particular gene comparison N-DGER values.

As discussed above, it is likely that in a prior art RT-PCR gene expression analysis assay of unknown cell samples, about ±8 percent difference in a particular gene or a standard assay E value is common for different unknown cell sample assays. Thus, in different unknown cell sample assays the particular gene assay E values or standard assay E values, may differ by as much as 16 percent. Further, in the same RT-PCR unknown cell sample assay tube, the particular gene and standard assay E values may differ by as much as 16 percent. For a first above-described unknown cell sample 30 cycle RT-PCR assay, where the standard E value is 8 percent less (0.828), than the particular gene E value of 0.9, the assay measured particular gene RN and abundance level values are overestimated, and deviate from biological accuracy by 3.2 fold. For this assay situation, the particular gene's true abundance level in all unknown cell samples is known to be one copy per cell. Therefore, the assay measured and overestimated particular gene abundance level is 3.2 copies per cell (CPC). For this assay, the assay measurement accuracy is to within ±1.2 fold. Thus, the measured assay accurate CPC value for the particular gene abundance level lies somewhere between 2.7 to 3.8 CPC. For a second above-described unknown cell sample 30 cycle RT-PCR assay, where the standard E value is 8 percent greater (0.972) than the particular gene 0.9 E value, then the assay measured particular gene RN value and abundance value is underestimated, and deviates from biological accuracy by 3 fold. Here, the assay measured particular gene abundance level is 0.33 CPC, and the accurate CPC value for this assay lies between 0.28 CPC and 0.4 CPC. For a third above-described 30 cycle RT-PCR assay, where the particular gene and standard assay E values are the same and equal to 0.9, then the assay measured particular gene RN value and abundance level values are biologically correct within the limits of the measurement accuracy of the assay. Thus, the biologically accurate particular gene abundance value lies between 0.83 CPC to 1.2 CPC.

RT-PCR measured particular gene N-DGER values for unknown cell sample comparisons are derived from the unknown cell sample RT-PCR measured particular gene RN or abundance level values. For the above-described unknown cell samples, the particular gene comparison T-DGER value equals one for all unknown cell sample comparisons. The particular gene comparison N-DGER value for, (the first above-described assay measured abundance value)÷(the third described assay measured abundance value) is equal to (3.2/1) or 3.2. The measurement accuracy of this particular gene N-DGER value is defined by the ratio of, (the measurement assay accuracy range of the first abundance value, i.e., 2.7 CPC to 3.8 CPC)÷(the measurement assay accuracy range of the third abundance value, i.e., 0.83 CPC to 1.2 CPC). Thus, the assay accurate particular gene N-DGER value lies between 2.3 CPC and 4.6 CPC. The (second abundance value)÷(the third abundance value) particular gene comparison N-DGER value is equal to (0.31/1) or 0.31, and the assay accurate N-DGER value lies between 0.23 CPC to 0.48 CPC. Further, the (first abundance value)÷(the second abundance value), particular gene comparison N-DGER value is equal to (3.2/0.31) or 10.3, and the assay accurate N-DGER value lies between 6.8 CPC and 13.6 CPC.

The ±8 percent value used in the above discussion is a conservative estimate of the one standard deviation value for the measurement accuracy of particular gene or standard E values for unknown cell sample ABI TaqMan RT-PCR gene expression quantitation assays. Further, ABI is part of the leading edge for the design, optimization, and use of RT-PCR assays for quantitating gene expression analysis, and it is highly likely that this ±8 percent measurement accuracy reflects the best, or close to the best, prior art assay E value measurement accuracy possible at this time for RT-PCR assays of all kinds. Note that this ±8 percent value is a one standard deviation value and that about one out of three measured E values will have a greater than ±8 percent deviation.

In order to know that a prior art RT-PCR assay measured particular gene N-DGER value is biologically accurate to within the measurement accuracy of the RT-PCR assay, it is necessary to know the assay values for the assay associated and compared E values to a very accurate degree. When the compared assay E values are the same, no normalization of the assay result for differences in the E values is required. The known degree of E value accuracy required in order to obtain RT-PCR assay measured particular gene N-DGER values which are known to be biologically accurate to within the measurement accuracy of the RT-PCR assay, can be illustrated. This is done below by using the above-described illustrative example of an RT-PCR assay which has a measurement accuracy of ±1.2 fold for assay measured particular gene RN and abundance values, and a measurement accuracy of ±1.44 for assay measured particular gene comparison N-DGER values. This means that the accurate N-DGER value for the assay is within ±1.44 fold of the assay measured N-DGER value. Note that a particular gene N-DGER value which is accurate for a particular RT-PCR assay, may not represent the biologically accurate N-DGER value for the cell sample comparison. For this illustration the biologically accurate particular gene abundance level is 1 CPC for all compared cell samples, and the biologically accurate particular gene comparison N-DGER value equals one for all cell sample comparisons. For this illustration it is assumed that the validity of the relationship (N-DGER)=(T-DGER) for a particular gene comparison can be affected only by a quantitative difference in the assay compared E values.

When for a cell sample particular gene comparison RT-PCR assay the compared assay E values are exactly the same, then the assay measured particular gene comparison N-DGER value of one is both biologically accurate, and assay accurate, to within ±1.44 fold. Here then, the biologically accurate N-DGER lies within the N-DGER value range 0.69 to 1.44, and the assay accurate N-DGER value also lies within the N-DGER value range of 0.69-1.44.

When for a cell sample particular gene comparison RT-PCR assay the compared assay E values differ by two percent, i.e., compared E values of (0.90/0.882), the assay measured particular gene comparison N-DGER value equals 1.33, and is not equal to the biologically accurate T-DGER value of one. This assay measured N-DGER value is assay accurate to within ±1.44 fold, and the assay accurate N-DGER value lies within the N-DGER value range of 0.92 to 1.92. Here the biologically accurate T-DGER value of one lies barely within the 0.92 to 1.92 measurement accuracy range of the assay. When the compared assay E values differ by three percent, the assay measured particular gene comparison N-DGER value equals 1.53, and the accurate assay N-DGER value lies within the N-DGER value range 1.06 to 2.2. Here the biologically accurate T-DGER value of one does not lie within the 1.06 to 2.2 measurement accuracy range of the assay. When the compared assay E values differ by six percent, the assay measured N-DGER value equals 2.3, and the accurate assay N-DGER value lies within the N-DGER value range of 1.6 to 3.3. The biologically correct T-DGER value does not fall within this assay measurement accuracy range.

An RT-PCR assay measurement accuracy of ±1.2 fold for particular gene RN values and ±1.44 fold for particular gene comparison N-DGER values, is often claimed by the prior art. For such an assay when the compared assay E values differ by three percent, the biologically accurate T-DGER value for the particular gene expression comparison does not fall within the assays measurement accuracy range. The context of the above illustration is an RT-PCR assay, which has an N-DGER measurement accuracy of ±1.44 fold. For RT-PCR assays which have an N-DGER measurement accuracy of ±2 fold or +4 fold, and the compared E values differ by six percent and 10 percent respectively, the biologically accurate T-DGER value for the particular gene comparison does not fall within the assays N-DGER value measurement accuracy range. For a prior art RT-PCR assay measured particular gene comparison N-DGER value, it cannot be known whether the biologically accurate particular gene comparison T-DGER value can fall within the assay's N-DGER value measurement accuracy range or not, absent knowledge of the quantitative difference in the compared AE•AE, and E assay values. The prior art determination of particular gene and standard assay E values was earlier discussed, and it appears that at best the prior art determined E values have a one standard deviation of around ±8 percent.

The above discussion concerns the likely differences in the assay compared AE•SE, AE•AE, and E values which occur for prior art RT-PCR assays, and the quantitative effect of such differences on the biological accuracy of the assay measured N-DGER values. From these discussions it can be concluded that prior art RT-PCR assay measured particular gene N-DGER values cannot be known to be correct or incorrect. It is also highly likely that for many if not most, such prior art measured N-DGER values, the compared E value differences are large enough to cause the N-DGER value to deviate from biological accuracy by 2 to 4 fold or more. Further, it is highly likely that just on the basis of the differences in compared E values, the third tacit assumption is invalid for many if not most prior art RT-PCR assays, and its validity cannot be known for any prior art RT-PCR assay. In addition, such differences in RT-PCR assay compared E values may be unavoidable since such differences of ±5 percent or below may be impractical or impossible to measure for the vast majority of RT-PCR unknown assay analyzes.

The above discussion has focused primarily on SGDS particular gene mRNA transcript comparisons. However, the discussion and conclusions apply directly to all SGDS, DGDS, and DGSS, RT-PCR assay analyzes for any type of analyzed RNA, including all types and kinds of prokaryotic, eukaryotic, viral, and synthetic RNAs such as rRNA, tRNA, mRNA, siRNA, miRNA, snoRNA, antisense RNA, and other known or unknown RNAs of any type. The discussion of the AE•AE and E values and the conclusions also apply directly to PCR assays of all kinds, whether RT-PCR or non-RT-PCR.

Is the Prior Art Belief That the (Assay NASR)=(ACR) Valid?

Prior art microarray and non-microarray gene expression analysis assays concern Type 1 or Type 2 LPN gene comparisons. Prior art generally believes that, for a microarray or non-microarray assay particular gene comparison, the ACR for the particular gene comparison in the assay, is equal to the particular gene comparison T-DGER which is present in the compared cell samples. Prior art further generally believes that the assay RASR result for each particular gene comparison must be adjusted or normalized in order to correct the assay RASR for prior art known assay biases or variables, before biologically meaningful interpretations of the assay RASR signal results can be made. In other words, the prior art believes the following. (a) The ACR, which is present in the assay hybridization solution for the particular gene comparison, is equal to the T-DGER for the gene comparison, which exists in the cell samples being compared. (b) The assay measured particular gene RASR value obtained in the assay must be corrected or normalized, so that the resulting assay NASR value equals the ACR for that gene comparison in the assay. (c) Since the ACR equals the biologically relevant T-DGER for the gene comparison, the prior art normalization must be done in order to obtain a biologically relevant, or meaningful, interpretation of the gene comparison assay result.

Prior art believes that normalization of microarray and non-microarray gene comparison results is necessary because of the existence of prior art known assay variables or biases, which influence the assay value of the RASR. These assay variables do not concern the biological difference in gene expression extents which exists in the compared cell samples for a mRNA of interest. These variables include, but are not limited to, biases associated with assay materials, assay processes, assay design, assay performance, and assay signal measurement. The aim of the prior art normalization process is to correct the assay signal results for those assay related differences which do not represent true gene expression differences in the compared cell samples.

A prior art microarray or non-microarray gene comparison assay NASR result for a particular gene comparison is derived from the assay RASR result by a prior art normalization process which normalizes for a variety of prior art known assay variables or biases. Prior art believes and practices that, when a particular gene comparison assay RASR result is normalized for prior art known assay variables, the resulting (assay NASR)=(ACR). Thus, prior art belief is that, a prior art normalized (assay NASR)=(assay N-DGER)=(ACR). Such prior art belief is valid only if all pertinent microarray or non-microarray assay variables have been taken into consideration in the prior art normalization process. Since the prior art believes and practices that, after prior art normalization of assay RASR results, the (assay NASR)=(ACR), by inference prior art believes that all of the pertinent microarray or non-microarray assay variables are known, and have been accounted for, during the normalization process.

Herein are described hidden multiple assay variables or biases which are not considered during prior art normalization of particular gene comparison assay RASR results, and which can cause the prior art belief that the (assay NASR)=(ACR), to be invalid. As discussed, these multiple hidden assay variables, which are not considered during prior art normalization, include one or more of the assay variable UNFs, MLDR, PL-HKR, PS-HKR, PSAR, PSSR, LLSR, SBNR, or SSAR. The prior art belief that the (prior art assay measured NASR)=(ACR), is valid for a particular gene comparison only when the assay value for each of the said UNFs which are pertinent to the assay are equal to one, or when the product of said pertinent NF's is equal to one, or when the product of the said assay pertinent UNFs is equal to one. This assumes other unknown variables do not exist which affect the relationship. It further assumes that the prior art produced assay NASR has been properly normalized for all prior art visible and considered assay variables, which are pertinent to the assay. There is good reason to believe that for many prior art particular gene comparisons, the assay value for one or more of these unconsidered assay variable NF's, deviates significantly from one. In this event the prior art belief that the (assay NASR)=(ACR), is invalid for many prior art microarray assays. However, absent knowledge not provided by the prior art, it cannot be known whether this relationship is valid or invalid for any particular prior art microarray assay produced N-DGER value.

Interpretation of Prior Art Produced NASR and N-DGER Assay Values when the (Assay NASR)≠(ACR).

The assay values for the unconsidered NFs MLDR, PL-HKR, PS-HKR, PSAR, PSSR, SBNR, and SSAR, can cause the prior art assay measured particular gene NASR value to not equal the ACR value for the particular gene in a Type 1 directly or indirectly labeled LPN assay. The assay values for the UNFs PL-HKR, PS-HKR, LLSR, SBNR, and SSAR can cause the prior art produced assay measured particular gene NASR value to not equal the ACR value for the particular gene in a Type 2 directly or indirectly labeled LPN assay. As discussed, the assay values for the UNFs SCR and PAFR, cannot influence whether the (NASR)=(ACR) for a particular gene comparison in an assay, or not. This discussion will concern only those prior art UNFs which can influence whether the (NASR)=(ACR) for a particular gene comparison in an assay. Further, for simplicity the discussion will focus on directly labeled LPN assays and the UNFs MLDR, PL-HKR, PS-HKR, PSAR, PSSR, and LLSR. However, the basic discussion and conclusions will apply directly to indirectly labeled L-LPN assays and their associated UNFs.

For a particular gene comparison assay, a situation where the (assay NASR)≠(ACR), occurs when the assay value for a pertinent UNF is not equal to one, and the product of the said pertinent UNF values, is not equal to one. For this discussion, the product of two or more pertinent UNF values is termed a UNF product, or UNFP.

For many prior art particular gene directly labeled LPN comparisons, there is good reason to believe that one or more of the MLDR, PL-HKR, PS-HKR, PSAR, PSSR, or LLSR UNF values is not equal to one, and that the product of these UNF values is also not equal to one. This discussion concerns the effect of the (assay NASR)≠(ACR) on the prior art interpretation of directly labeled LPN assay measured NASR values for particular gene comparisons. Because, by definition, the (assay NASR)=(assay N-DGER), for a particular gene comparison, and because prior art generally reports gene comparison results in terms of the assay measured and normalized N-DGER values, this discussion will be in terms of the prior art interpretation of N-DGER values. Further, because prior art belief is that the (assay N-DGER)=(T-DGER) for a particular microarray gene comparison, the discussion will focus on the prior art interpretation of a prior art produced N-DGER value in situations where, unknown to the prior art, the assay value for a pertinent UNF and UNFP, is not equal to one. In the context of this discussion, a pertinent UNF or UNFP for a SGDS Type 1 LPN gene comparison involves one or more of the MLDR, PL-HKR, PS-HKR, PSAR, and PSSR UNFs, while a pertinent UNF or UNFP for a SGDS Type 2 LPN gene comparison involves one or more of the PL-HKR, PS-HKR, PSSR, LLNR, LLSR UNFs. In such a situation, when it is known that the assay value for UNFP≠1, then the (assay NASR)=(assay N-DGER)≠(ACR), for either a Type 1 or Type 2 LPN assay.

The interpretation of the prior art produced assay N-DGER for such a situation, can be illustrated for a microarray SGDS Type 1 or Type 2 LPN gene comparison by considering an idealized microarray assay. For this idealized assay it is assumed that: Cell Sample 1 and Cell Sample 2 Gene B mRNA LPNs are compared; the Gene B T-DGER is known; the Gene B LPN ACR is known, the EA Rule is practiced and the assay values for SCR and PAFR equal one, and therefore in the assay the Gene B LPN ACR is equal to the Gene B T-DGER; the assay value for one or more of the UNFs, MLDR, PL-HKR, PS-HKR, PSAR, PSSR, LLSR UNFs, and the UNFP assay value, is not equal to one; the prior art normalization process corrects for all other pertinent assay variables. For simplification, the illustrations are presented and discussed in terms of, the prior art interpretation when an assay UNFP value is not equal to one. These illustrations will apply to both SGDS Type 1 and Type 2 LPN comparisons. Table 23 illustrates the prior art interpretation of a prior art produced particular gene comparison assay N-DGER result, by comparing such a result to the known T-DGER for the assay. It is clear from these illustrations that, when the assay UNFP ≠1 the prior art N-DGER value is erroneous, since it does not equal the ACR or T-DGER of the gene comparison. In addition, certain of the erroneous prior art assay N-DGER values are associated with regulation direction miscalls, or RDMs (see Table 23 vi-ix).

TABLE 23
Prior Art Interpretation of Prior Art Gene B mRNA LPN Comparison When
the Assay UNFP Is Not Equal To One

| | | | | | Prior Art | |
|---|---|---|---|---|---|---|
| | | | | | N-DGER | |
| | | | | Assay^{(c)} | Assessment of | |
| | | | | Observed Prior | Gene B | |
| Known Assay | | Known Assay | Assay UNFP | Art Normalized | Regulation^{(d)} | |
| T-DGER^{(a)} | | ACR^{(b)} | Value | N-DGER Value | Activity | Reality |
| (i) | 1 | 1 | 1 | 1 | No Change | No Change |
| (ii) | 4 | 4 | 4 | 16 | Up 16x | Up 4x |
| (iii) | 4 | 4 | 2 | 8 | Up 8x | Up 4x |
| (iv) | 4 | 4 | 1 | 4 | Up 4x | Up 4x |
| (v) | 4 | 4 | 0.5 | 2 | Up 2x | Up 4x |
| (vi) | 4 | 4 | 0.25 | 1 | No Change | Up 4x |
| (vii) | 4 | 4 | 0.248 | 0.99 | Down 1.01x | Up 4x |
| (viii) | 4 | 4 | 0.125 | 0.5 | Down 2x | Up 4x |
| (ix) | 4 | 4 | 0.05 | 0.2 | Down 5x | Up 4x |

^{(a)}All ratios are in terms of (Cell Sample 1 parameter) ÷ (Cell Sample 2 parameter).
^{(b)}In all examples the assay SCR = 1 and PAFR = 1.
^{(c)}By definition, the (assay NASR) = (assay N-DGER).
^{(d)}Up = upregulated; Down = down regulated; x = fold change in gene expression.

Thus, a prior art produced particular gene comparison N-DGER result which is associated with a UNFP≠is very likely to be erroneous with regard to the magnitude of the difference in gene expression extents in the compared cell samples, and can be associated with an RDM. Table 23 indicates that the further the UNFP assay value deviates from one, the greater the deviation of the assay N-DGER and the assay NASR, from the T-DGER value, and the gene comparison ACR. Such behavior is similar to that seen for the earlier discussed assay variable UNF, the SCR, which is described in Tables 4, 5, 6, and 7. In addition, Table 23 indicates that UNFP≠1 related RDM results, do not occur at every UNFP ≠1 assay value, but occur over a specified range of UNFP≠1 assay values. Again, such behavior is similar to that seen for the earlier discussed SCR UNF related RDM's, described in Tables 4, 5, 6, and 7. The earlier discussions and characteristics of the SCR related erroneous N-DGER and RDM assay results, are directly applicable to the illustrations to Table 23.

Each of the assay variable UNFs MLDR, PL-HKR, PS-HKR, PSAR, and PSSR is a non-global NF. Consequently, in one assay different gene comparisons can have different assay values for one UNF. In contrast, the LLSR is a global assay variable NF, and therefore has only one assay value, which applies to each particular gene comparison in the assay.

As discussed earlier, the SCR does affect the ACR of the assay, while each of the MLDR, PL-HKR, PS-HKR, PSAR, PSSR, LLNR, and LLSR can affect the assay RASR value for a particular gene comparison, but do not affect the ACR of the assay. As also discussed, there is good reason to believe for many particular prior art gene comparisons, that the assay UNFP value associated with the pertinent MLDR, PL-HKR, PS-HKR, PSAR, PSSR, and LLSR UNFs is not equal to one. Thus, many prior art produced gene comparison assay NASR and N-DGER values are associated with a situation where the (assay NASR)=(assay N-DGER)≠(ACR), and the prior art produced assay NASR and N-DGER results are therefore incompletely normalized. Because of this, it cannot be known for any particular prior art gene comparison whether the relationship (assay NASR)=(ACR), is valid or not, since for any particular prior art gene comparison, the prior art produced assay NASR may or may not equal the ACR. Absent some knowledge of the particular gene comparison assays UNFP value, there is no way to determine such validity. As a consequence, prior art produced assay NASR and N-DGER values are not interpretable with regard to biological accuracy. In addition, many of these prior art N-DGER results are likely to be associated with RDMs. As a consequence of this, the data mining and systems biology analyzes of prior art produced assay NASR and N-DGER values, also produces results which cannot be known to be correct or incorrect, and are therefore not interpretable with regard to the general pattern, or patterns of gene expression changes. Such data mining analyzes includes scatterplots, principle component analysis, expression maps, pathway analysis, cluster analysis, self-organizing maps, and others.

Note that the above conclusions also apply to prior art indirectly labeled L-LPN assay results.

Overall Effect of MLDR, PL-HKR, PS-HKR, PSAR, PSSR, LLSR, SBNR, and SSAR UNFs On the Relationship (NASR)=(N-DGER)=(ACR).

The assay values for the UNFs MLDR, PL-HKR, PS-HKR, PSAR, PSSR, SBNR, and SSAR may be pertinent for a prior art Type 1 gene expression comparison assays. The assay values for the UNFs PL-HKR, PS-HKR, PSSR, LLSR, SBNR, and SSAR may be pertinent for a prior art Type 2 gene expression comparison assay. Note that assay variables associated with label density are associated with the PL-HKR and PS-HKR UNFs. This discussion is intended to illustrate the effect of all of the assay pertinent UNFs on the said relationship. For simplicity, the discussion will focus on assays using directly labeled LPNs. However, the general basic discussion and conclusions apply directly to indirectly labeled L-LPN assays.

For a particular gene comparison in an assay, absent other compensating assay factors, when one of the UNFs≠1, then (N-DGER)=(NASR)≠(ACR), for that particular gene comparison in the assay. The assay value for each different UNF which is pertinent to a particular gene comparison in an assay, has an independent effect on the assay RASR for that particular gene comparison, and on the (N-DGER)=(NASR)=(ACR) relationship. Therefore, the overall effect of all of these UNFs on the assay N-DGER value, or (N-DGER)=(NASR)=(ACR) relationship, for a particular gene comparison in the assay, is equal to the product of the assay values of all of the UNF values which are associated with the particular gene comparison. Here, this product is termed the UNF product or UNFP for the particular gene comparison. For a particular gene comparison in an assay, when the UNFP≠1, then the N-DGER=NASR≠ACR. When for a particular gene comparison, two or more of the UNFs do not equal one, the individual UNF values may interact to produce a UNFP value which is much larger than any individual UNF value, or much smaller than any individual UNF value. Prior art does not determine the assay UNFP value for each particular gene comparison in an assay. Therefore, prior art produced particular gene NASR or N-DGER values are not normalized for the UNFP. Prior art believes and practices that such a prior art produced and normalized N-DGER value is equal to the assay ACR value and the T-DGER value, and is therefore biologically accurate. However, absent other compensating assay effects, when the assay UNFP≠1 for a particular prior art gene comparison the prior art produced and normalized NASR and N-DGER values are incompletely normalized, and do not equal the ACR value for the particular gene comparison in the assay. The N-DGER or NASR value for a particular gene comparison will deviate from the assay ACR value for the particular gene expression, by the same magnitude that the UNFP value deviates from one.

There is good reason to believe that for prior art microarray gene expression comparison assay, UNFP≠1 assay values are not uncommon for many particular gene comparisons. Practically, such a UNFP≠1 is relevant for prior art assay RASR, NASR, or N-DGER normalization, only if the assay UNFP deviates from one significantly. Such deviations have relevance when the magnitude of the deviation of the UNFP from one is large enough to significantly affect the value of the prior art produced RASR, NASR, or N-DGER for a particular gene comparison, when the RASR, NASR, or N-DGER value is normalized for the UNFP. Such normalization is done using the relationship, (T-DGER)=(N-DGER)÷(UNFP).

Many prior art microarray assays claim to produce assay measured particular gene NASR values, which are accurate to within about ±1.2 to ±2 fold (152, 192-197). These prior art assay measured particular gene NASR values are not normalized for assay UNFs and therefore are incompletely normalized when the assay UNFP value is not equal to one. The magnitude of the deviation from one for commonly occurring UNF≠1 values for each different UNF is estimated below for prior art microarray particular gene comparison assay. The deviation from one for commonly occurring UNF≠1 values is estimated below for the various UNFs which may be pertinent to a prior art directly labeled or indirectly labeled LPN assay.

It is known that compared particular gene LPN TNC values and nucleotide lengths can differ by 5 to 10 fold or more, and often differ by 2 to 4 fold. Such differences are caused by differences in the purity and state of degradation of the compared cell sample RNAs, the type of primer used to produce the compared cell sample LPN preps, and common imperfections associated with producing the cell sample LPN preps. Differences in the purity or state of degradation of the RNA are common for compared cell samples. It is also known that compared cell sample LPN TPN values can differ by 5 to 10 fold or more, and often differ by 2 to 4 fold. Such differences are caused by the state of degradation of the compared cell sample RNAs, and the type of primer used to produce the compared cell sample LPN preps. Differences in the state of degradation of compared cell sample RNAs are common. It is further known that particular gene ECDP nucleotide complexities can be about 30 or 60 nucleotides for oligonucleotide microarrays, and roughly 300 to 1200 nucleotides or longer, for cDNA microarrays. The above issues were discussed earlier. All of these assay factors contribute to the MLDR UNF assay value. As indicated in Table 20, different combinations of such factors can cause the assay MLDR value to deviate from one by as much as 10-20 fold in a plausible prior art assay. Here, it is reasonable to believe that an assay particular gene MLDR value which deviates from one by 2 to 4 fold, is not uncommon for prior art gene expression comparison assays. Here, a deviation of 3 fold is a reasonable estimate. As indicated above, it is known that compared cell sample LPN nucleotide lengths can differ by 5-10 fold or more, and often differ by 2 to 4 fold. As discussed, the kinetics of hybridization of the LPN with the spot immobilized CDP is inversely proportional to the square root of difference in compared LPN nucleotide lengths. Here, it is reasonable to believe that particular gene PL-HKR assay values, which deviate from one by 1.5 fold, will not be uncommon.

As discussed earlier, differences in compared cell sample LPN nucleotide lengths cause significant differences in compared cell sample LPN nucleotide sequences, and can cause significant differences in compared cell sample LPN nucleotide composition. In addition, differences in the cell sample LPN LD values, which often occur, can magnify the nucleotide sequence difference effect on the hybridization kinetics of the compared cell sample LPNs. Such effects could cause the assay PS-HKR value to deviate from one by as much as 5-10 fold or more. Given the prior art practices concerning the LPN production process it is reasonable to believe that a deviation from one of 2 fold to 4 fold or so for assay PS-HKR values is not uncommon. Here, it is reasonable to estimate that an assay PS-HKR value which deviates from one by 2 fold is not unusual.

As discussed earlier, differences in compared cell sample LPN nucleotide sequence and/or nucleotide composition can cause significant differences in compared cell sample LPN PSA values. In addition, differences in the cell sample LPN LD values can amplify the cell sample LPN PSA differences. Such effects could cause the assay PSAR value to deviate from one by as much as 4-6 fold or more. Given the prior art practices concerning the production of compared cell sample LPN preps, it is reasonable to believe that an assay PSAR value, which deviates from one by 2 to 3 fold is not uncommon. Here, it is reasonable to estimate that an assay PSAR value which deviates from one by 2 fold is not uncommon.

As discussed earlier, differences in compared cell sample LPN LD values can cause significant differences in compared cell sample hybridized LPN duplex stabilities. Such effects would be amplified by differences in cell sample LPN nucleotide lengths, LPN nucleotide sequences, and nucleotide compositions, and by the use of high stringency assay conditions designed to enhance LPN specificity of reaction. Very little is known concerning PSSR value of prior art assays. However, given the prior art practices concerning the production of cell sample LPN preps, PSSR values which deviate from one by 2 to 3 fold would not be surprising. In this context, it is reasonable to estimate that PSSR assay values which deviate from one by 1.5 fold are not uncommon.

A small fraction of prior art microarray gene expression comparison assays compare cell sample Type 2 LPN preps. For these assays the LLNR is readily known and is often equal to one. However, even in a situation where the assay LLNR=1, and each compared LPN is associated with the same label molecule, it cannot always be assumed that the assay LLSR=1. When the LLNR=1, and each compared LPN prep is labeled with the same radioactive isotope, then it can be assumed that the assay LLSR=1. When the LLNR 1, and each compared LPN prep is labeled with a different radioactive isotope, then the LLSR cannot be assumed to equal one. Further, when the LLNR=1, and each compared LPN prep is labeled with the same fluorescent dye, such as Cy3, or a different fluorescent dye, then it cannot be assumed that the assay LLSR value is equal to one. Differences in the process of producing LPNs can cause differences in the signal activity per dye molecule for compared cell sample LPNs labeled with the same fluorescent dye. Further, different dyes are often associated with different signal activities PCR dye molecule. It also cannot be assumed that because the LLNR≠1, the LLSR≠1. The LLSR value for an assay can only be known by measurement. It is reasonable to believe that a deviation from one of 1.5 to 3 fold for assay LLSR values is not uncommon. Here, it is reasonable to estimate that an assay LLSR value which deviates from one by 2 fold is not uncommon.

The UNFs SBNR and SSAR are associated only with assays comparing indirectly labeled L-LPNs. Such assays are also associated with other UNFs. The majority of prior art indirect label L-LPN assays involve Affymetrix assays. For these assays it is reasonable to believe that assay SBNR values which deviate from one by 1.5 fold or so are not uncommon, and that the assay SSAR values deviate from one by a smaller amount.

The vast majority of prior art microarray gene expression comparison assays compare Type 1 directly or indirectly labeled LPN molecules. The large majority of these Type 1 assays use oligo dT primer produced cell sample cDNA or cRNA preps. All of the above-described UNFs, except the LLNR and LLSR, may be pertinent to such Type 1 assays, as well as to Type 1 assays associated with random primed directly or indirectly labeled LPN preps. The overall effect of these UNFs which are associated with an assay, on the relationship (N-DGER)=(NASR)=(ACR) for a particular gene comparison, and the significance of any such effect, is discussed below. This discussion is primarily in terms of directly labeled LPN assays.

Each of the above-described estimates of commonly occurring prior art UNF assay values is large enough to significantly change the prior art measured N-DGER value by normalizing for the UNF. As an example, normalization of an N-DGER value of two, with a UNF value which deviates from one by 1.5 fold, will result in a newly normalized N-DGER value of 1.33 or 3. Such a change has a significant effect on the prior art N-DGER value, and its biological accuracy. The aggregate effect of these UNFs on a prior art measured particular gene N-DGER value can be smaller, or much larger, than 1.5 fold. Table 24 illustrates how the UNFP for these UNF estimates might affect the biological accuracy of prior art measured particular gene N-DGER values. In addition, the effect of the UNFP on the relationship (N-DGER)=(NASR)=(ACR), is illustrated. For Table 24 it is assumed that for each particular gene comparison, (ACR)=(T-DGER).

TABLE 24
Overall Effect of UNFs on Particular Gene N-DGER For Type 1 LPNs

| | | | | | | | ^{(a)}Deviation of Prior Art | |
|---|---|---|---|---|---|---|---|---|
| | | | | | | | N-DGER Value From | |
| | Estimated Value for UNF | | | | | Assay | Assay Value For | |
| | MLDR | PL-HKR | PS-HKR | PSAR | PSSR | UNFP | ACR | ^{(b)}T-DGER |
| (i) | 1 | 1 | 1 | 1 | 1 | 1 | 1 | 1 |
| (ii) | 3 | 1.5 | 2 | 2 | 1.5 | 27 | 27 | 27 |
| (iii) | 0.33 | 0.67 | 0.5 | 0.5 | 0.67 | 0.037 | 27 | 27 |
| (iv) | 0.33 | 0.67 | 0.5 | 2 | 1.5 | 0.33 | 3 | 3 |
| (v) | 3 | 0.67 | 2 | 0.5 | 0.67 | 2 | 2 | 2 |
| (vi) | 0.33 | 1.5 | 0.5 | 2 | 1.5 | 0.74 | 1.35 | 1.35 |
| (vii) | 3 | 0.67 | 2 | 0.5 | 0.67 | 1.35 | 1.35 | 1.35 |
| (viii) | 3 | 1.5 | 0.5 | 0.5 | 0.67 | 0.75 | 1.33 | 1.33 |
| (ix) | 0.33 | 0.67 | 2 | 2 | 1.5 | 0.75 | 1.33 | 1.33 |

^{(a)}For this table it is assumed that for each particular gene comparison, that (ACR) = (T-DGER)
^{(b)}Normalize N-DGER for UNFP by using the relationship (T-DGER) = (N-DGER) ÷ (UNFP).

Note that most of these UNFs are associated with non-global assay variables, and as such each particular gene comparison in an assay may have a different assay value for a particular UNF. Table 24 (i) illustrates a situation where the UNF values for a particular gene comparison in an assay are all equal to one. Here, there is no effect on the N-DGER value. Table 24 (ii)-(vii) illustrate the effect of different combinations of the estimated commonly occurring values. Table 24 (ii) and (iii) represent situations where all of the deviations from one, are either greater than one, or less than one, respectively. In each case, the prior art measured N-DGER value deviates from the ACR and the T-DGER by 27 fold. Here, depending on what the assay situation is for a particular gene, the actual particular gene T-DGER could be equal to (N-DGER÷0.037) or (N-DGER÷27), a 729 fold difference. Table 24 (viii) and (ix) illustrate the minimum effect of these estimated UNF values. Here, the actual particular gene T-DGER value could be equal to (N-DGER÷1.33), or (N-DGER÷0.75) a 1.8 fold difference. Note that only a few of the many possible UNF combinations are illustrated here.

The assay values of MLDR, PL-HKR, PS-HKR, and PSSR are all influenced by differences in the compared cell sample LPN nucleotide lengths. Absent such a difference for oligo dT or specific gene primed cell sample LPNs, then the assay values for MLDR, PL-HKR, and PS-HKR are all equal to one. When there is a nucleotide length difference for compared cell sample oligo dT or specific gene primed particular gene LPNs, both the MLDR and the PL-HKR assay values will be either greater than one or less than one. This is illustrated in Table 24 (ii)-(iv) and (viii) and (ix).

Absent some knowledge of the UNF assay values for each particular gene comparison, which is not provided by the prior art, the assay UNFP value cannot be known. Therefore, it cannot be known whether the prior art measured and normalized N-DGER value is biologically accurate or not. It is highly likely, however, that many if not most such prior art produced N-DGER values, are associated with a situation where (N-DGER)=(NASR)≠(ACR).

The above discussions concerning the effects of the various UNFs on the relationship (NASR)=(N-DGER)=(ACR), primarily focused on SGDS assay comparisons of particular gene mRNA transcripts. However, these discussions apply directly to SGDS, DGDS, and DGSS assay comparisons of viral, prokaryotic, eukaryotic, and standard RNA transcripts of all kinds. This includes all types of rRNA, tRNA, mRNA, siRNA, miRNA, snoRNA, antisense RNA, and other known and unknown RNAs.

E. Effect of All UNFs on the Validity of Prior Art Produced N-DGER Values when it is Not Assumed That (ACR)=(T-DGER) or (ACR)=(NASR)=(N-DGER).

Prior art believes and practices that prior art microarray and non-microarray assay measured and normalized particular gene N-DGER values are biologically accurate, within the accuracy of the assay. Many prior art microarray assays claim to be able to obtain particular gene N-DGER values which are biologically accurate to within ±1.2 to ±2 fold (152, 192-197). These prior art particular gene N-DGER values are normalized for one or more of the prior art considered assay variable NFs, ARR, TSAR, C-HKR, PCR E value, spatial, print tip, print plate, intensity, scale, background, random noise, and image analysis.

Previous sections have examined the validity of two key prior art assumptions which must be true for the microarray or non-microarray assay, in order for prior art assay produced particular gene N-DGER values to be biologically correct. One key prior art assumption and belief specifies that for a particular gene comparison, (ACR)=(T-DGER). A second key assumption and belief specifies that for a particular gene comparison, (ACR)=(NASR)=(N-DGER). Thus, prior art believes and practices that for a particular gene comparison, (N-DGER)=(NASR)=(ACR)=(T-DGER), or briefly that (N-DGER)=(T-DGER).

In order to separately evaluate the validity of each of these key prior art beliefs, previous sections have examined the effect of UNFs which are pertinent to each key assumption, on the validity of the key assumption, when the other key assumption is valid. The UNFs SCR, and PAFR, can influence the validity of the key assumption (ACR)=(T-DGER). One section examined the effect of SCR and PAFR on the validity of (ACR)=(T-DGER), when it was assumed that the other key assumption, (ACR)=(NASR)=(N-DGER) was valid. The UNFs MLDR, PL-HKR, PS-HKR, PSAR, PSSR, LLSR, SBNR, and SSAR, as well as the CNF AE•AER, can influence the validity of the key assumption (ACR)=(NASR)=(N-DGER). A second section examined the effect of the AE•AER, MLDR, PL-HKR, PS-HKR, PSAR, PSSR, LLSR, SBNR, and SSAR on the validity of (ACR)=(NASR)=(N-DGER), when it was assumed that the key assumption, (ACR)=(T-DGER), was valid.

The present discussion concerns the effect of all of the UNFs, SCR, PAFR, MLDR, PL-HKR, PS-HKR, PSAR, PSSR, LLSR, SBNR, and SSAR, which are pertinent to an assay on a particular gene N-DGER value, when it is not assumed that (ACR)=(T-DGER), or that (ACR)=(NASR)=(N-DGER). The effect of the pertinent UNFs on microarray and non-microarray Type 1 and Type 2 LPN particular gene N-DGER values will be discussed. For a particular gene Type 1 LPN assay, one or more of the UNFs SCR, PAFR, MLDR, PL-HKR, PS-HKR, PSAR, PSSR, SBNR, SSAR, are pertinent. Here, the assay UNFP is termed a Type 1 UNFP. For a particular gene Type 2 LPN assay, one or more of the UNFs, SCR, PAFR, PL-HKR, PS-HKR, PSSR, LLSR, SBNR are pertinent. Here, the assay UNFP is termed a Type 2 LPN UNFP.

As discussed, there is good reason to believe that for many prior art microarray and non-microarray assays, UNFP≠1 assay values are not uncommon for particular gene comparisons. Prior art produced N-DGER values are not normalized for the UNFs SCR, PAFR, MLDR, PL-HKR, PS-HKR, PSAR, PSSR, LLSR, SBNR, or SSAR. Therefore, a prior art particular gene N-DGER value which is associated with an assay UNFP≠1 value is incompletely normalized, and is likely to be biologically inaccurate. In order to obtain a biologically accurate value, such an N-DGER value must be normalized for the UNFP value. Such normalization is done using the relationship (T-DGER)=(N-DGER)÷(UNFP). For an assay measured particular gene RASR value, the normalization is done using the relationship (normalized DGER)=(RASR)÷(UNFP).

The assay value for each different UNF, which is pertinent to a particular gene comparison in an assay, has an independent effect on the biological accuracy of a UNFP normalized assay result for that particular gene comparison. Therefore, the overall effect of all pertinent UNFs on a particular gene comparison assay result, is equal to the product of the assay values for all of the pertinent UNF values which are associated with the particular gene comparison. The resulting UNFP value can be much larger or much smaller than any individual UNF value.

Prior art does not measure, or take into consideration during the prior art normalization process for a particular gene comparison, the assay values for the UNFs SCR, PAFR, MLDR, PL-HKR, PS-HKR, PSAR, PSSR, LLSR, SBNR, and SSAR. Therefore, prior art produced particular gene N-DGER values are not normalized for these UNFs. As discussed, there is good reason to believe that for many prior art produced particular gene comparisons, the UNFP≠1. Consequently, absent other compensating factors, for these particular gene comparisons the N-DGER values are unlikely to be biologically accurate and cannot be known to be biologically accurate or inaccurate, and may be associated with RDMs.

The vast majority of prior art microarray gene expression comparison assays are associated with oligo dT primed fluorescent Type 1 LPNs, and determine the N-DGER values from the particular gene NASR values produced for the compared cell samples. The effect of the assay UNFPs on the N-DGER values produced by such an assay can be illustrated by considering a microarray assay, which has the following characteristics. (a) The gene expression activity of Gene B in Cell Samples 1 and 2 are compared using oligo dT primed Type 1 directly labeled fluorescent LPN preps. Gene B is actively expressed in each cell sample, and the (Cell Sample 1/Cell Sample 2) Gene B T-DGER=4. (b) The prior art normalization process corrects each compared cell sample's Gene B RASR value for all pertinent prior art considered assay variables to produce a Gene B NASR value for each cell sample. The cell sample Gene B NASR values are then compared to produce a prior art Gene B N-DGER value. (c) The value for each UNF associated with the assay is the earlier determined estimated value. Such estimated values for each UNF are believed to occur commonly for prior art microarray assays, and are believed to be conservative estimates. Here, the estimates for the SCR assume that tacit assumptions one and three are invalid, and pertinent to the assay. The estimated SCR values used are 6 or 0.17, and 1.5 or 0.67. The estimated values for other UNFs are: PAFR=0.75 or 1.33; PL-HKR=0.67 or 1.5; PS-HKR=0.5 or 2; PSAR=0.5 or 2; PSSR=0.67 or 1.5. (d) It is assumed that the prior art considered NFs and prior art UNFs are the only assay variables which affect the biological accuracy of the prior art particular gene N-DGER values.

This illustration is presented in Table 25. Table 25 illustrates only a few of the many possible combinations of UNF values, and the resulting UNFP values. Table 25 (i) and (ii) illustrate the maximum deviation of the prior art N-DGER value from biological accuracy for these UNF values. This occurs when all of the UNFs have assay values greater than, or less than, one. Here, the maximum deviation ranges from 54 fold to 215 fold, depending on the SCR value used. Table 25 (iii) and (iv) illustrate that certain combinations of UNF values give UNFP values of close to one, and therefore prior art N-DGER values which are close to being biologically accurate. Table 25 (ii) and (vi) indicate UNF combinations, which result in RDMs.

TABLE 25
Effect of UNFP On Prior Art Produced Gene B N-DGER Values: Oligo dT
Primed Fluorescent Type 1 LPN Microarray Comparisons

| | | | | | | | | | | | | ^{(d)}Assessment of |
|---|---|---|---|---|---|---|---|---|---|---|---|---|
| | | | | | | | | | | | | Direction of |
| | | | | | | | | | | | | Gene B |
| | ^{(a)}UNF Assay Value | | | | | | | | | ^{(b)}Prior Art | | Regulation |
| | S C R | P A F R | M L D R | PL- H K R | PS- H K R | P S A R | P S S R | Gene B UNFP | Known Gene B T-DGER | Produced Gene B N-DGER Value | $\begin{matrix}{}^{( c )} Normalization \\ Deficit \\ \frac{( N \text{-} DGER )}{( T \text{-} DGER )}\end{matrix}$ | Change From Prior Art N-DGER Value |
| | 1 | 1 | 1 | 1 | 1 | 1 | 1 | 1 | 4 | 4 | 1 | Up 4x |
| (i) | 6 | 1.33 | 3 | 1.5 | 2 | 2 | 1.5 | 215 | 4 | 860 | 215 | Up 860x |
| | 1.5 | 1.33 | 3 | 1.5 | 2 | 2 | 1.5 | 54 | | 216 | 54 | Up 216z |
| (ii) | 0.17 | 0.75 | 0.33 | 0.67 | 0.5 | 0.5 | 0.67 | 0.0047 | 4 | 0.019 | 0.0047 | Down 54x |
| | 0.67 | 0.95 | 0.33 | 0.67 | 0.5 | 0.5 | 0.67 | 0.019 | | 0.076 | 0.019 | Up 13x |
| (iii) | 6 | 1.33 | 0.33 | 0.67 | 0.5 | 2 | 0.67 | 1.2 | 4 | 4.8 | 1.2 | Up 4.8x |
| | 1.5 | 0.75 | 3 | 1.5 | 0.5 | 0.5 | 0.67 | 0.85 | | 3.4 | 0.85 | Up 3.4x |
| (iv) | 0.17 | 0.75 | 3 | 1.5 | 2 | 2 | 0.67 | 1.5 | 4 | 6 | 1.5 | Up 6x |
| | 0.67 | 0.75 | 3 | 1.5 | 0.5 | 0.5 | 1.5 | 0.85 | | 3.4 | 0.85 | Up 3.4x |
| (v) | 6 | 1.33 | 3 | 1.5 | 0.5 | 2 | 0.67 | 24 | 4 | 96 | 4 | Up 96x |
| | 1.5 | 0.75 | 3 | 1.5 | 0.5 | 2 | 1.5 | 7.6 | | 30 | 7.6 | Up 30x |
| (vi) | 0.17 | 0.75 | 0.33 | 0.67 | 2 | 2 | 1.5 | 0.17 | 4 | 0.68 | 0.17 | Down 1.5x |
| | 0.67 | 0.75 | 0.33 | 0.67 | 0.5 | 2 | 0.67 | 0.07 | | 0.28 | 0.07 | Down 3.6x |

(a) All ratios have Sample 1 parameter in numerator.
(b) (N-DGER) = (UNFP) (T-DGER).
(c) (Normalization Deficit) = (UNFP) = (N-DGER) ÷ (T-DGER).
(d) Up = upregulated; Down = downregulated; x = Fold Change in Expression Extent.

Table 25 illustrates the difficulty in interpreting whether a prior art microarray assay measured particular gene N-DGER is biologically accurate or not. Prior art does not determine the assay values for the UNFs, and a prior art produced N-DGER value is not normalized for the assay UNFP value. In addition, there is good reason to believe that many, if not most, prior art microarray assays are associated with UNFP values, which deviate significantly from one. Table 25 indicates that conservative estimates for microarray assay UNF values can result in many prior art N-DGER values which deviate significantly from biological accuracy. Absent knowledge of the actual UNF and UNFP assay values, it cannot be known whether a particular prior art assay is associated with a UNFP≠1 or not.

While each of the UNF assay values has an independent effect on the biological accuracy of a N-DGER value, the assay values of certain of these UNFs are coordinated. As an example, the MLDR and PL-HKR UNFs are both strongly influenced by differences in the nucleotide lengths of the compared cell sample LPNs. Here, if the assay value for the MLDR>1, then it is likely that the assay value for the PL-HKR<1. Depending on the assay details, this could result in a (MLDR×PL-HKR) product value which is smaller than the MLDR value. The PSAR UNF is directly and strongly influenced by label density differences for the compared cell sample LPNs. The PS-HKR and PSSR UNFs are indirectly influenced by label density differences for compared cell sample LPNs, and can be strongly influenced at high LD levels. Under certain assay conditions, the UNF values for the PS-HKR and PSSR will be positively coordinated, but the PSAR UNF value will be negatively coordinated with the PS-HKR and PSSR UNF values. This is unlikely to occur for most prior art assays. The MLDR and PL-HKR UNFs are not coordinated with either the PSAR UNF, or the PS-HKR and PSSR UNFs. The SCR and PAFR UNFs are not coordinated with each other, or any other UNF.

A minority fraction of prior art microarray assays compare cell sample randomly primed Type 1 LPNs. Most of these assays utilize fluorescent labeled Type 1 LPNs. For such assays, differences in the nucleotide lengths of the compared cell sample Type 1 LPNs are significantly less than for oligo dT primed Type 1 fluorescent LPN comparisons. As a result, for these assays the likely assay values for the MLDR, PL-HKR, PS-HKR, and PSAR UNFs are significantly smaller than for the oligo dT primed situation. Because of this, it is reasonable to believe that MLDR values which deviate from one by 1.5 fold, are not uncommon for prior art randomly primed fluorescent Type 1 LPN comparisons. Further, it is reasonable to believe that PL-HKR UNF values which deviate from one by 1.2 fold, and PS-HKR and PSAR UNF values which deviate from one by 1.5, are not uncommon for prior art random primed fluorescent Type 1 LPN comparisons. Note that under certain less common assay conditions, much larger deviations from one can occur for MLDR and PL-HKR, and PS-HKR UNF values. For random primed Type 1 fluorescent LPN comparisons, the cell sample cDNA YF values tend to be higher than for oligo dT primed LPNs. Because of this it is reasonable to believe that SCR values which deviate from one by 4.5 fold, are not uncommon for prior art random primed fluorescent Type 1 LPN comparisons. Note that under certain less common conditions, much larger deviations from one can occur for the SCR. Random priming does not affect the estimates for PAFR.

Non-microarray gene expression assays employing northern blot, dot blot, and nuclease protection methods often utilize 3′ and labeled radioactive Type 2 LPNs. A small fraction of prior art microarray gene expression assays compare Type 2 LPNs, and these are generally radioactive or fluorescent labeled LPNs. As discussed earlier, the MLDR is not pertinent for these Type 2 LPN microarray assays and the PSSR is very unlikely to be pertinent for these Type 2 LPN assays. Further, the PSAR is also not pertinent for these assays, and is replaced by the UNF LLSR. Note that the LLSR is a global assay UNF. It is reasonable to believe that a Type 2 LLSR value, which deviates from one by 2 fold, is not uncommon. The use of Type 2 LPNs does not affect the estimated values for the SCR, PAFR, PL-HKR, or PS-HKR UNFs.

Note that for Table 25 and the discussion thus far, the N-DGER values have been determined by comparing particular gene normalized assay signals (NAS) which are derived from raw assay signals (RAS). A very small fraction of prior art microarray gene expression comparison assays, produce particular gene N-DGER values by first determining the mRNA abundance values for a particular gene in each compared cell sample, and then comparing these mRNA abundance values. In this situation all three tacit assumptions are pertinent to the assay, and it is reasonable to believe that the estimated SCR value deviates from one by 9 fold for an oligo dT primed LPN assay, and by 6 fold for a random primed LPN microarray assay.

The overall pattern of the UNFP value effects is essentially the same for oligo dT, SG, and random primed Type 1 LPN comparisons, and oligo dT or SG primed Type 2 LPN comparisons. Some UNF combinations result in very high or low UNFP values. These values indicate that the prior art N-DGER value can commonly deviate from biological accuracy by a large factor. A few UNF combinations result in UNFP values, which equal one or nearly one. Such UNFP values indicate that the prior art N-DGER value is biologically accurate, or nearly so. Many of the different UNF combinations have a UNFP value which deviates significantly from one. For most potential assay UNF combinations, the UNFP value, i.e., the normalization deficit, is large enough to indicate that the prior art N-DGER value is biologically inaccurate to a significant degree. Normalizing for even small UNFP values can have a significant effect on the prior art interpretation of the prior art microarray produced N-DGER values. This is discussed later.

The above discussions are directly applicable to cell sample comparisons using fluorescent or radioactive LPNs. For cell sample radioactive LPN comparison, the commonly occurring estimated prior art assay UNF values are similar to the earlier discussed fluorescent LPN comparisons, with the exception of the PSSR. It is highly likely that the PSSR UNF value equals one for the vast majority of radioactive particular gene comparisons.

As discussed earlier in detail, there is good reason to believe that for many prior art RT-PCR assays of all kinds, UNFP≠1 assay values are common for particular gene comparisons. Therefore, these prior art produced RT-PCR measured particular gene comparison N-DGER values which are associated with UNFP≠1 values are incompletely normalized and are likely to be biologically inaccurate. In order to obtain biologically accurate N-DGER values such prior art measured incompletely normalized N-DGER values must be normalized for the UNFP≠1 values, as described earlier. These common RT-PCR assay UNFP≠1 values occur even though most of the UNFs, which are pertinent to microarray assays, are not pertinent for RT-PCR assays. This is discussed below.

The UNFs, which are directly pertinent to RT-PCR assays, are the SCR and PAFR. Each of these UNFs can affect the validity of the relationship (N-DGER)=(ACR)=(T-DGER) for a particular gene comparison in an RT-PCR assay. Neither of these UNFs affects the validity of the relationship (N-DGER)=(NASR)=(ACR). Prior art believes and practices that adequate control and normalization procedures are available to endure the validity of this second relationship. For this discussion it is assumed that the relationship (N-DGER)=(NASR)=(ACR) is valid for RT-PCR assays, and that only a deviation of the SCR and/or PAFR assay value from one can affect the biological accuracy of the RT-PCR measured N-DGER value.

The effect of the SCR and PAFR UNF assay values, and the resulting UNFP, on the biological accuracy of prior art RT-PCR assay produced particular gene comparison N-DGER values, is discussed below. This can be illustrated using an RT-PCR assay, which has the following characteristics. (a) The gene expression activity of Gene B in Cell Samples 1 and 2 are compared. The (Cell Sample 1/Cell Sample 2) Gene B T-DGER=4. (b) Cell sample T-RNAs or isolated mRNAs are compared using the EA Rule. (c) SG primers are used in the RT step. (d) A particular gene N-DGER value is determined from either measured assay particular gene mRNA transcript number values or equivalents, or particular gene mRNA abundance values. Equivalents refers to assay measured NAS values. (e) The prior art assay measured Gene B N-DGER values are corrected for all pertinent prior art considered assay variable NFs, including the AE•SER and AE•AER. (f) The assay value for SCR or PAFR which is associated with the Gene B comparison, is determined from the earlier estimated value for the deviation of the UNF value from one, which is believed to commonly occur for many prior art particular gene comparisons. These estimated UNF values are different for different RT-PCR assay situations, and the estimated values for each different assay situation are presented in Tables 26 and 27.

Table 26 illustrates RT-PCR assays, which analyze cell sample T-RNA using SG primers. Here, the PAFR=1, and the only UNF which can influence the biological accuracy of the prior art measured N-DGER values, and the prior art interpretation of the N-DGER values, is the SCR. Table 27 illustrates RT-PCR assays which analyze cell sample isolated mRNA. Here, the PAFR≠1, and both the SCR and PAFR UNFs influence the biological accuracy of the N-DGERs, and the prior art interpretation of N-DGER values. As discussed, the assay SCR value can be influenced by the invalidity of one or more of the three tacit assumptions. When the prior art N-DGER value is determined from the compared cell sample's measured mRNA transcript number values, or equivalents, only tacit assumptions one and three are pertinent to the assay. When the N-DGER value is derived from the compared cell sample mRNA abundance values, all three of the tacit assumptions are pertinent for the assay.

TABLE 26
Effect of UNFP On Prior Art RT-PCR Produced Gene B N-DGER Values:
Specific Gene Primed LPN

| | | | | | | | | ^{(d)}Assessment of |
|---|---|---|---|---|---|---|---|---|
| | | | | | | | | Direction of |
| | | ^{(a)}Estimated UNF | | | | | | Gene B |
| | | Assay Value | | | | ^{(b)}Prior Art | | Regulation |
| Cell Sample RNA Type | N-DGER Value Determined From | ^{(e)}SCR | PAFR | Gene B UNFP | Known Gene B T-DGER Value | Produced Gene B N-DGER Value | $\begin{matrix}{}^{( c )} Normalization \\ Deficit \\ \frac{( ( N \text{-} DGER )}{( T \text{-} DGER )}\end{matrix}$ | Change From Prior Art N-DGER Value |
| (i) T-RNA | mRNA | 1 | 1 | 1 | 4 | 4 | 1 | Up 4x |
| | Transcript | 6 | 1 | 6 | 4 | 24 | 6 | Up 24x |
| | Number | 1.5 | 1 | 1.5 | 4 | 6 | 1.5 | Up 6x |
| | Values or | 0.66 | 1 | 0.66 | 4 | 2.7 | 0.67 | Up 2.7x |
| | Equivalents | 0.17 | 1 | 0.17 | 4 | 0.68 | 0.17 | Down 1.5x |
| (ii) T-TNA | mRNA | 9 | 1 | 9 | 4 | 36 | 9 | Up 36x |
| | Abundance | 4 | 1 | 4 | 4 | 16 | 4 | Up 16x |
| | Values | 2.3 | 1 | 2.3 | 4 | 9.2 | 2.3 | Up 9.2x |
| | | 1 | 1 | 1 | 4 | 4 | 1 | Up 4x |
| | | 0.44 | 1 | 0.44 | 4 | 1.8 | 0.44 | Up 1.8x |
| | | 0.25 | 1 | 0.25 | 4 | 1 | 0.25 | Unchanged |
| | | 0.11 | 1 | 0.11 | 4 | 0.44 | 0.11 | Down 2.3x |

(a) All ratios have Sample 1 parameter in numerator.
(b) (N-DGER) = (UNFP) (T-DGER).
(c) (Normalization Deficit) = (UNFP) = (N-DGER) ÷ (T-DGER)
(d) Up = Upregulated; Down = Downregulated; x = Fold Change in Expression Extent.
(e) SCR values from Table 11. Here, tacit assumption two is not pertinent to the assay for (i) and is pertinent for (ii).

TABLE 27
Effect of UNFP On Prior Art RT-PCR Produced Gene B N-DGER Values:
Specific Gene Primed LPN

| | | | | | | | | ^{(d)}Assessment of |
|---|---|---|---|---|---|---|---|---|
| | | | | | | | | Direction of |
| | | ^{(a)}Estimated UNF | | | | | | Gene B |
| | | Assay Value | | | | ^{(b)}Prior Art | | Regulation |
| Assayed Cell Sample RNA Type | N-DGER Value Determined From | ^{(e)}SCR | PAFR | Gene B UNFP | Known Gene B T-DGER Value | Produced Gene B N-DGER Value | $\begin{matrix}{}^{( c )} Normalization \\ Deficit \\ \frac{( N \text{-} DGER )}{( T \text{-} DGER )}\end{matrix}$ | Change From Prior Art N-DGER Value |
| (i) Isolated | mRNA | 6 | 1.33 | 8 | 4 | 32 | 8 | Up 32x |
| mRNA | Transcript | 6 | 0.75 | 4.5 | 4 | 18 | 4.5 | Up 18x |
| | Number | 1.5 | 1.33 | 2 | 4 | 8 | 2 | Up 8x |
| | Values or | 1.5 | 0.75 | 1.1 | 4 | 4.4 | 1.1 | Up 4.4x |
| | Equivalents | 0.67 | 1.33 | 0.9 | 4 | 3.6 | 0.9 | Up 3.6x |
| | | 0.67 | 0.75 | 0.5 | 4 | 2 | 0.5 | Up 2x |
| | | 0.11 | 1.33 | 0.15 | 4 | 0.6 | 0.15 | Down 1.5x |
| | | 0.11 | 0.75 | 0.083 | 4 | 0.33 | 0.75 | Down 3x |
| (ii) Isolated | mRNA | 9 | 1.33 | 12 | 4 | 48 | 12 | Up 48x |
| mRNA | Abundance | 9 | 0.75 | 6.8 | 4 | 27.2 | 6.8 | Up 27.2x |
| | Values | 4 | 1.33 | 5.3 | 4 | 21.2 | 5.3 | Up 21.2x |
| | | 4 | 0.75 | 3 | 4 | 12 | 3 | Up 12x |
| | | 2.3 | 1.33 | 3 | 4 | 12 | 3 | Up 12x |
| | | 2.3 | 0.75 | 1.73 | 4 | 6.9 | 1.73 | Up 6.9x |
| | | 1 | 1.33 | 1.33 | 4 | 5.3 | 1.33 | Up 5.3x |
| | | 1 | 0.75 | 0.75 | 4 | 3 | 0.75 | Up 3x |
| | | 0.45 | 1.33 | 0.6 | 4 | 2.4 | 0.6 | Up 2.4x |
| | | 0.45 | 0.75 | 0.34 | 4 | 1.36 | 0.34 | Up 1.36x |
| | | 0.25 | 1.33 | 0.33 | 4 | 1.36 | 0.33 | Up 1.36x |
| | | 0.25 | 0.75 | 0.19 | 4 | 0.76 | 0.19 | Down 1.3x |
| | | 0.11 | 1.33 | 0.15 | 4 | 0.6 | 0.15 | Down 1.67x |
| | | 0.11 | 0.75 | 0.083 | 4 | 0.33 | 0.083 | Down 3x |

^{(a)-(e)}See Table 26 footnotes (a)-(e).

This is true for cell sample T-RNA or isolated mRNA comparisons which use SG, oligo dT, or random primed LPNs. The Table 26 and 27 illustrations reflect this situation. The derivation of the estimated SCR values used in these illustrations was discussed earlier as part of the discussion concerning Table 11. For both Tables 26 and 27, the UNFP value is generally dominated by the estimated SCR value, even when the PAFR≠1. The overall pattern of UNFP value effects is essentially the same for the Table 26 and Table 27 illustrations, and further is similar to the earlier discussed microarray assay overall pattern. Most of the estimated assay UNFP values deviate significantly from one, and some UNFP values differ very significantly from one. This indicates that most of the N-DGER values deviate significantly from biological accuracy. However, some of the estimated assay UNFP values are equal to one, or nearly one, which indicates that the associated N-DGER values are biologically accurate, or nearly so. Even small UNFP values can have a significant effect on the prior art interpretation of the prior art RT-PCR produced N-DGER values. This will be discussed later.

Tables 26 and 27 specifically concern the comparison of SG primed cell sample LPNs. However, the general aspects of these tables and the discussion associated with them, applies directly to oligo dT and random primed cell sample LPN comparisons. While the magnitude of the SCR and PAFR UNFPs can be affected by the type of primer used, and the type of cell sample RNA analyzed, the general conclusions apply to the use of any primer type or cell sample RNA. Similarly, these discussions apply to DGDS and DGSS particular gene RNA of any kind comparisons.

Tables 26 and 27 illustrate the difficulty in interpreting whether a prior art RT-PCR assay measured particular gene N-DGER is biologically accurate or not. Prior art does not determine the assay values for the UNFs, and a prior art produced N-DGER value is not normalized for the assay UNFP value. In addition, there is good reason to believe that many, if not most, prior art RT-PCR assays are associated with UNFP values, which deviate significantly from one. Tables 26 and 27 indicate that conservative estimates for RT-PCR assay UNF values can result in many prior art N-DGER values which deviate significantly from biological accuracy. Absent knowledge of the actual UNF and UNFP assay values, it cannot be known whether a particular prior art assay is associated with a UNFP≠1 or not.

Almost all microarray assays and all RT-PCR assays do not directly compare cell sample T-RNA or mRNA, but compare cell sample RNA equivalents such as cDNA or cRNA. In contrast, essentially all prior art northern blot, dot blot, and nuclease protection assays, directly compare cell sample T-RNAs or mRNAs. As discussed earlier, there is good reason to believe that many prior art northern blot, dot blot, and nuclease protection assays, are associated with UNFP≠1 values. Therefore, prior art produced northern blot, dot blot, and nuclease protection, particular gene N-DGER results which are associated with UNFP≠1 values are incompletely normalized and are likely to be biologically inaccurate. In order to obtain biologically accurate N-DGER values, such incompletely normalized N-DGER values must be normalized for the UNFP≠1 values, as described earlier. The UNFs, which are directly pertinent to the northern blot, dot blot, and nuclease protection assays, are the SCR and PAFR. Each of these UNFs can affect the validity of the relationship (N-DGER)=(ACR)=(T-DGER) for a particular gene comparison in a northern blot, dot blot, or nuclease protection assay. Neither of these UNFs affects the validity of the relationship (N-DGER)=(NASR)=(ACR) for these assays. Prior art believes and practices that adequate control and normalization procedures are available to ensure the validity of this second relationship for these assays. Here, it has been assumed that the second relationship is valid for prior art northern blot, dot blot, and nuclease protection, assay measured particular gene N-DGER values.

The effect of the SCR and PAFR UNF assay values, and the resulting UNFP value, on the biological accuracy of prior art northern blot, dot blot, and nuclease protection assay produced particular gene N-DGER values, is discussed below. For simplification, the discussion will focus on the nuclease protection assay. However, the discussion will apply directly to northern blot and dot blot assays. This can be illustrated using a nuclease protection assay, which has the following characteristics. (a) The gene expression activity of Gene B in Cell Samples 1 and 2 are compared. The (Cell Sample 1/Cell Sample 2) Gene B T-DGER=4. (b) Cell sample T-RNAs or isolated mRNAs are compared using the EA Rule. (c) A single preparation of Gene B LPN is used for the assay. (d) A particular gene N-DGER value is determined from either measured assay particular gene mRNA transcript number values or equivalents, or measured particular gene mRNA abundance values. (e) The prior art assay measured N-DGER values are corrected for all pertinent prior art considered assay variable NFs. (f) The assay value for SCR or PAFR which is associated with the Gene B comparison, is determined from the earlier estimated value for the deviation of the UNF value from one, which is believed to commonly occur for many prior art particular gene comparisons. These estimated UNF values are different for different assay situations, and the estimated SCR and PAFR values for each assay situation are presented in Tables 28 and 29. For simplification, nuclease protection assays are referred to as NP assays.

Table 28 illustrates nuclease protection (NP) assays, which analyze cell sample T-RNA. Here, the assay PAFR=1, and the only UNF which can affect the biological accuracy of the prior art measured N-DGER values, and the prior art interpretation of the prior art N-DGER values, is the SCR. Table 29 illustrates NP assays which analyze cell sample isolated mRNA. Here, the PAFR≠1, and both the SCR and PAFR assay values can influence the biological accuracy of the prior art measured N-DGER values, and the prior art interpretation of the N-DGER values. As discussed, the assay SCR value can be influenced by the invalidity of one or more of the three tacit assumptions. Here, for an NP assay which analyzes cell sample T-RNA and determines the particular gene N-DGER value from compared cell sample mRNA transcript number values, or equivalents, only the first tacit assumption is pertinent to the NP assay SCR value.

TABLE 28
Effect of UNFP On Prior Art Nuclease Protection Assay N-DGER Values:
Comparing Cell Sample T-RNA

| | | ^{(a)}Estimated UNF | | | | | | ^{d}Assessment of |
|---|---|---|---|---|---|---|---|---|
| | | Assay Value | | | | ^{(b)}Prior Art | | Direction of Gene |
| Cell Sample RNA Type | N-DGER Value Determined From | ^{(e)}SCR | PAFR | Gene B UNFP | Known Gene B T-DGER Value | Produced Gene B N-DGER Value | $\begin{matrix}{}^{( c )} Normalization \\ Deficit \\ \frac{( N \text{-} DGER )}{( T \text{-} DGER )}\end{matrix}$ | B Regulation Change From Prior Art N-DGER Value |
| (i) T-RNA | mRNA | 1 | 1 | 1 | 4 | 4 | 1 | Up 4x |
| | Transcript | 3 | 1 | 3 | 4 | 12 | 3 | Up 12x |
| | Number | 0.33 | 1 | 0.33 | 4 | 1.3 | 0.33 | Up 1.3x |
| | Values or | | | | | | | |
| | Equivalents | | | | | | | |
| (ii) T-RNA | mRNA | 4.5 | 1 | 4.5 | 4 | 18 | 4.5 | Up 18x |
| | Abundance | 2 | 1 | 2 | 4 | 8 | 2 | Up 8x |
| | Values | 0.5 | 1 | 0.5 | 4 | 2 | 0.5 | Up 2x |
| | | 0.22 | 1 | 0.22 | 4 | 0.88 | 0.22 | Down 1.1x |

^{(a)-(e)}See Table 26 footnotes (a)-(e).

TABLE 29
Effect of UNFP On Prior Art Nuclease Protection Assay N-DGER Values:
Comparing Cell Sample Isolated mRNA

| | | | | | | | | ^{(d)}Assessment of |
|---|---|---|---|---|---|---|---|---|
| | | ^{(a)}Estimated UNF | | | | | | Direction of |
| | | Assay Value | | ^{(b)}Prior Art | | | Gene B | |
| Cell Sample RNA Type | N-DGER Value Determined From | ^{(e)}SCR | PAFR | Gene B UNFP | Known Gene B T-DGER Value | Produced Gene B N-DGER Value | $\begin{matrix}{}^{( c )} Normalization \\ Deficit \\ \frac{( N \text{-} DGER )}{( T \text{-} DGER )}\end{matrix}$ | Regulation Change From Prior Art N-DGER Value |
| (i) Isolated | mRNA | 3 | 1.33 | 4 | 4 | 16 | 4 | Up 16x |
| mRNA | Transcript | 3 | 0.75 | 2.3 | 4 | 9.2 | 2.3 | Up 2.3x |
| | Number | 0.33 | 1.33 | 1 | 4 | 4 | 1 | Up 4x |
| | Values of | 0.33 | 0.75 | 0.25 | 4 | 1 | 0.25 | Unchanged |
| (ii) Isolated | mRNA | 4.5 | 1.33 | 6 | 4 | 24 | 6 | Up 24x |
| mRNA | Abundance | 4.5 | 0.75 | 3.4 | 4 | 13.6 | 3.4 | Up 13.6x |
| | Values | 2 | 1.33 | 2.67 | 4 | 10.7 | 2.67 | Up 10.7x |
| | | 2 | 0.75 | 1.5 | 4 | 6 | 1.5 | Up 6x |
| | | 0.5 | 1.33 | 0.67 | 4 | 2.7 | 0.67 | Up 2.7x |
| | | 0.5 | 0.75 | 0.38 | 4 | 1.5 | 0.38 | Up 1.5x |
| | | 0.22 | 1.33 | 0.29 | 4 | 1.2 | 0.29 | Up 1.2x |
| | | 0.22 | 0.75 | 0.17 | 4 | 0.68 | 0.17 | Down 1.5x |

^{(a)-(e)}See Table 26 footnotes (a)-(e).

Further, the PAFR=1 for this assay. For such a NP assay then, only the assay SCR UNF value influences the biological accuracy of the N-DGER value. The Table 28 (i) illustration reflects this situation. Table 28 (ii) illustrates a situation where T-RNA is compared, but the N-DGER value is determined from NP assay measured particular gene mRNA abundance values. Here, tacit assumptions one and two are pertinent to the NP assay, and the PAFR=1. Table 29 illustrates the NP assay analysis of isolated cell sample mRNA. Here, the PAFR≠1.

The overall pattern of the estimated UNFP value effects on prior art NP assay N-DGER values is essentially the same for the Table 28 and 29 illustrations, and further is similar to the earlier discussed microarray and RT-PCR overall patterns. Most of the estimated UNFP values deviate significantly from one, and some UNFP values differ very significantly from one. Thus, most of the N-DGER values associated with these assays deviate significantly from biological accuracy, while some of the estimated UNFP assay values are equal to one, or nearly one, and are therefore associated with biologically accurate, or nearly biologically accurate, N-DGER values. Even small UNFP values can have a significant affect on the prior art interpretation of the prior art produced N-DGER values. This is discussed below.

Tables 28 and 29 illustrate the difficulty in interpreting whether a prior art NP, northern blot, or dot blot, assay measured particular gene N-DGER value is biologically accurate or not. Prior art does not determine the assay values for the SCR or PAFR UNFs, and a prior art N-DGER value is not normalized for these UNFs. In addition, there is good reason to believe that many, if not most, prior art NP, northern blot, and dot blot, assays are associated with UNFP values, which deviate significantly from one. Tables 28 and 29 indicate that conservative estimates of NP assay UNF values can result in many prior art N-DGER values, which deviate significantly from biological accuracy. However, absent some knowledge of the actual UNF and UNFP values which are associated with the assay, it cannot be known whether a particular prior art NP, northern blot, or dot blot, is associated with a UNFP≠1, or not.

A gene expression comparison assay UNFP≠1, which is unknown to the prior art, can affect the validity of the prior art analysis and interpretation of the biological accuracy of prior art measured particular gene N-DGER values in multiple ways. First, when the magnitude of the deviation of the prior art unknown UNFP is large enough, the prior art measured N-DGER value can be known to be biologically inaccurate. Second, even when the prior art unknown UNFP≠1 value is relatively small, the N-DGER value cannot be known to be biologically accurate or inaccurate. Third, even when the prior art unknown UNFP value is relatively small, prior art interprets and misidentifies genes which are significantly expressed as being unregulated, and other gene which are unregulated as being significantly expressed. Fourth, when the magnitude of the prior art unknown UNFP is large enough, prior art interprets and misidentifies upregulated genes as being downregulated or vice versa. Fifth, when the prior art unknown UNFP≠1, prior art often interprets and misidentifies genes in one cell sample as being actively expressed and upregulated, relative to the same genes in the second cell sample which are not measured by the assay as being actively expressed, but which in reality, are actively expressed to an equal or greater extent in the second cell sample.

When the UNFP≠1 for a particular gene comparison prior art measured N-DGER, the N-DGER value is incompletely normalized. Here, a prior art measured N-DGER, which is associated with an assay UNFP≠1, is incorrect and must be normalized for the assay UNFP≠1 value. Here, a prior art deficiently normalized N-DGER is termed a DN-DGER, while a UNFP normalized DN-DGER is termed an improved normalized DN-DGER, or IN-DGER. The DN-DGER normalization is done using the relationship (IN-DGER)=(DN-DGER)÷(UNFP).

The effect of such a prior art unknown UNFP≠1 value on the validity of the prior art analysis and interpretation of the biological accuracy of prior art measured N-DGER values is illustrated below for microarray, RT-PCR, and NP assays. For this discussion, it will be useful to describe certain characteristics of a typical prior art microarray, RT-PCR, or NP assay cell sample comparison. For most gene expression comparison assays the great majority of prior art measured particular gene N-DGERs have small values which generally range from around 0.33 to 3 (7). This occurs for most prior art prokaryote and eukaryote cell comparisons. For mammalian cell comparisons, typically thousands of different gene comparisons have prior art measured N-DGER values of 0.33 to 3. Further, it is known that for a typical mammalian cell sample comparison, 12,000 or so different genes are expressed in each compared cell sample, and well over half of these genes are expressed in both cell samples as low abundance mRNA transcripts. This indicates that for a mammalian cell comparison assay, over 6,000 different genes will have prior art measured N-DGER values of 0.33 to 3. In addition, the abundance of different commonly expressed low abundance mRNA transcripts is similar, but not necessarily the same, in each compared cell sample. This large overlap between commonly expressed low abundance mRNA populations of different related cell types, is common for other eukaryotes as well as prokaryotes. Generally, prior art microarray and RT-PCR assays are claimed to be able to measure biologically accurate N-DGER values to within ±2 fold or less. Certain prior art microarray and RT-PCR assays are claimed to be able to measure biologically accurate N-DGER values to within about ±1.2 fold. Prior art northern blot and dot blot assays are often regarded as being semi-quantitative. However, prior art NP assays are also capable of measuring accurate particular gene N-DGER values to within about ±1.2 fold (144).

The effect of a prior art unknown UNFP≠1 value on the validity of the prior art analysis and interpretation of the biological accuracy of prior art microarray measured particular gene N-DGER values, can be illustrated by considering the following assay situation. (a) Unknown to the prior art, the assay UNFP value equals 0.75 or 0.17. The UNFP is associated with only global assay variables. (b) For the microarray assay mammalian cell comparison over 6,000 genes have prior art assay measured N-DGER values of 0.33 to 3. Further, 500 of these particular gene comparisons have prior art measured N-DGER values of between 1.51 to 2, while a different 500 genes have N-DGER values of between 0.376 and 0.499. For the assay, 5,000 genes have N-DGERs of between 0.5 and 2.
(c) The prior art specifies that the prior art microarray assay can accurately measure a particular gene N-DGER value to within ±2 fold. Further, the prior art specifies that for this assay a particular gene with a measured N-DGER value of >2 or <0.5, is significantly differentially expressed, while a particular gene with a measured N-DGER value of <2 or >0.5 is not significantly differentially expressed. (d) The assay N-DGER values have the compared Cell Sample 1 parameters in the numerator and the Cell Sample 2 parameters in the denominator. (e) Using the specified significance criteria, the prior art interpretation of the assay N-DGER values, is that the 500 genes with assay measured N-DGER values of 0.376 to 0.499, are significantly differentially expressed, while the 500 different genes with N-DGER values of 1.51 to 2 are not significantly differentially expressed. Further, the prior art interprets the Cell Sample 1 genes, which are associated with the 0.376 to 0.499 N-DGER values, as being significantly downregulated, relative to the expression of the same genes in Cell Sample 2. In addition, the prior art interprets the Cell Sample 1 genes associated with the 1.51 to 2 N-DGER values, as being unregulated, relative to the expression of the same genes in Cell Sample 2. As discussed, the prior art measured deficiently normalized N-DGER is termed a DN-DGER, while a UNFP normalized prior art DN-DGER is termed an improved normalized DGER or IN-DGER.

It is reasonable to believe that, unknown to the prior art, assay UNFP values of 0.17, or so are not unusual for prior art microarray and non-microarray assays. A prior art example where, unknown to the prior art, a global assay variable UNFP which deviates from one by 10 fold, was discussed earlier. As described for this illustration, 500 of the gene comparisons in the assay have prior art measured DN-DGER values, which range from 1.51-2. The prior art interpretation of these values indicates that all 500 of these genes are unregulated because they have prior art measured DN-DGER values of 2 or less, and greater than 0.5. When these DN-DGER values are normalized for the assay UNFP=0.17 value, which is unknown to the prior art, all 500 of these genes have IN-DGER values of 8.9 to 11.8. By the prior art assay standard of significance then, all of these genes are very significantly differentially expressed, and the Cell Sample 1 genes are all very significantly upregulated. This is in contrast to the prior art interpretation, which indicated that all of these genes were unregulated. As further described, 500 other genes in this microarray assay have prior art measured DN-DGER values, which range from 0.376 to 0.499. The prior art interpretation of these values indicates that all 500 of these genes are significantly differentially expressed, and that the Cell Sample 1 genes are all downregulated. When these DN-DGER values are normalized for the assay UNFP=0.17 value, all 500 of these gene have IN-DGER values of greater than 2, which range from 2.2 to 2.9. By the prior art standard of significance then, all of these genes are significantly expressed, and the Cell Sample 1 genes are upregulated. This is in contrast to the prior art interpretation that all 500 of these genes are downregulated in Cell Sample 1. As further described for this illustration, a total of 5000 genes have prior art measured DN-DGER values of between 0.5 and 2. The prior art interpretation of these values is that none of these 5000 genes is significantly differentially expressed. When these DN-DGER values are normalized for the assay UNFP 0.17 value, all 5000 of these genes have IN-DGER values of 2.9 to 11.8. By the prior art standard of significance then, all of these genes are significantly differentially expressed, and all 5000 genes are upregulated in Cell Sample 1. This is in contrast to the prior art interpretation, which indicates that all of these genes are unregulated. The above discussion clearly indicates that when the magnitude of the deviation of the assay UNFP value from one is large enough, the prior art measured DN-DGER values can be known to be biologically inaccurate. In addition, genes which are prior art interpreted to be upregulated in a cell sample are actually downregulated, and vice versa, and genes which are prior art interpreted as being unregulated are actually upregulated or down-regulated.

Even when the prior art unknown UNFP≠1 value is small, it can affect the validity of the prior art analysis and interpretation of the biological accuracy of the prior art measured DN-DGER values. As described, 500 of the genes in the assay have prior art measured DN-DGER values, which range from 1.51 to 2. The prior art interpretation indicates that all 500 of these genes are unregulated because they have prior art measured DN-DGER values of 2 or less, and greater than 0.5. When these DN-DGER values are normalized for an assay UNFP=0.75 value, which is unknown to the prior art, all 500 of these genes have IN-DGER values of greater than 2, and these values range from 2.01 to 2.67. By the prior art assay standard of significance then, all 500 of these genes are significantly differentially expressed, and upregulated, with regard to Cell Sample 1 genes. This is in contrast to the prior art interpretation that all 500 genes were unregulated.

As further described, 500 other genes in the microarray assay have prior art measured DN-DGER values, which range from 0.376 to 0.499. The prior art interpretation indicates that all 500 of these genes are significantly differentially expressed, and that the Cell Sample 1 genes are all downregulated, relative to the same genes in Cell Sample 2. When these DN-DGER values are normalized for the assay UNFP=0.75 value, all 500 of these genes have DN-DGER values of 0.5 or greater, and these values range from 0.501 to 0.67. By the prior art assay standard of significance then, all 500 of these genes are not significantly differentially expressed, and are therefore unregulated. This is in contrast to the prior art interpretation that all 500 of these genes were significantly expressed before UNFP normalization, and that the Cell Sample 1 genes were all downregulated, relative to the same genes in Cell Sample 2. The above discussion illustrates that for a prior art microarray assay which has a measurement accuracy of ±2 fold, a small UNFP=0.75 value which deviates from 1 by 1.33 fold, can significantly affect the validity of the prior art interpretation of many prior art measured particular gene DN-DGER values. Because the prior art does not determine the assay UNFP values associated with the particular gene comparisons in an assay, the prior art cannot know that the prior art interpretation of the biological accuracy of these DN-DGER values is inaccurate, and that the prior art interpretation misidentifies many genes as being unregulated which are significantly differentially expressed, or regulated, and also misidentifies many genes as being significantly differentially expressed, or regulated, which are unregulated.

The above discussion on the effect of prior art unknown UNFP≠1 values on the validity of the prior art interpretation of prior art microarray produced DN-DGER values, applies directly to prior art produced RT-PCR and NP DN-DGER values, as well as to SGDS, DGDS, and DGSS particular gene RNA of all kinds transcript comparisons.

Prior art unknown small and large assay UNFP≠1 values affect the validity of the prior art analysis and interpretation of prior art measured particular gene N-DGER values. Unknown to the prior art, such small or large assay UNFP values can cause prior art measured particular gene N-DGER values: (a) To be biologically inaccurate. (b) To be misidentified as being associated with unregulated genes when the genes are actually regulated. (c) To be misidentified as being associated with regulated genes when the genes are actually unregulated. (d) To be misidentified as being associated with upregulated genes when the genes are actually downregulated, and vice versa. (e) In addition, such prior art unknown small and large UNFP≠1 values cause the occurrence of UNFP≠1 related false negatives for genes which are present in one of the compared cell samples. These false negatives are associated exclusively with the genes of only one of the compared cell samples, and these genes are not detected as being actively expressed in the assay, while the same genes in the other compared cell sample are detected as being actively expressed in the assay, and the mRNA abundance of the undetected genes, is equal to or greater than the mRNA abundance of the detected genes. Under certain assay conditions, large numbers of such UNFP≠1 related false negative values can occur for an assay. Each UNFP≠1 related false negative is associated with an RDM. Such false negatives occur primarily for those genes whose mRNA abundance values are near the cell samples just detectable abundance level for the assay. Such false negatives have been discussed extensively elsewhere herein.

For such prior art assays with low or high prior art unknown assay UNFP values, absent some knowledge of the assay UNFP value, it cannot be known whether the prior art interpretation regarding the biological accuracy of the prior art assay measured N-DGER values, is valid or not. It is very likely that assay UNFP values which deviate significantly from one are common for all kinds of prior art gene expression comparisons, and it is known that prior art gene expression comparison practice does not determine assay UNFP values. Because of this, it cannot be known for any specific prior art assay measured particular gene N-DGER value, whether it is biologically accurate or not. In other words, prior art measured particular gene N-DGER values are uninterpretable with regard to biological accuracy, and such results are often largely uninterpretable with regard to regulation direction changes. Further, the extent of occurrence of UNFP≠1 related false negative results and their associated RDMs, cannot be known.

It is necessary to determine the assay UNFP values for gene expression comparison assays of all kinds in order to obtain particular gene N-DGER values, which are improved relative to prior art produced particular gene N-DGER values. Knowledge of the assay UNFP values for particular gene comparisons provides information necessary for producing and interpreting particular gene N-DGER values which can be known to be improved in normalization and biological accuracy. Further, such knowledge can be used to improve the overall process of normalization and interpretation of assay measured particular gene RASR values, and to generally produce particular gene N-DGER values which are known to be more completely and accurately normalized, than prior art produced particular gene N-DGER values. Knowledge of the assay UNFP value can be used in the following ways in order to produce particular gene N-DGER values, which are improved relative to prior art produced particular gene N-DGER values. (i) Such knowledge can be used to identify those assay situations, which require no normalization for assay UNFP values. (ii) Such knowledge can be used to identify those assay situations, which require normalization for the assay UNFP value, and provides the assay UNFP value for doing the normalization. (iii) Such knowledge can be used to produce completely, or more completely normalized assay measured particular gene N-DGER values. (iv) Such knowledge can be used in conjunction with the quantitative value for the measurement accuracy of the assay, to better interpret the significance of the assay measured and normalized particular gene N-DGER values, with regard to biological accuracy. (v) Such knowledge can be used to estimate the frequency of occurrence of UNFP≠1 false negative results and their associated RDMs. (vi) Such knowledge can be used to identify the mRNA or RNA abundance levels in the compared cell sample, which are associated with the occurrence of false negative results.

Note that for simplicity, in this overall discussion on the effect of the UNFP it has generally been assumed that the illustrative UNFP values are associated only with global assay variables. As discussed earlier, in reality the UNFP values are often associated with non-global assay variables.

The above discussion concerning UNFPs concerned SGDS comparisons of particular gene mRNA transcripts. This discussion also applies directly to all SGDS, DGDS, and DGSS particular gene comparisons of viral, prokaryotic, eukaryotic, and standard RNAs of all kinds. This includes all types of rRNA, tRNA, mRNA, siRNA, miRNA, snoRNA, antisense RNA, and other known or unknown RNAs.

F. Effect of UNFP Assay Values on the Interpretation of Prior Art Microarray Data Analysis and Data Mining Analysis Results and Systems Biology Analysis Results.

There is good reason to believe that many, if not most, particular prior art produced microarray and corroborative gene comparison assay N-DGER values are associated with assay UNFP≠1 values. Consequently, such N-DGER values are erroneous with regard to the magnitude of gene expression, and may be erroneous with regard to the direction of gene regulation change, which is implied by the N-DGER value, thereby resulting in RDMs. In a cell sample gene comparison assay such erroneous N-DGER results and RDMs can occur for any particular gene comparison in the assay, and at any RNA abundance level in a cell sample. Because the unconsidered NFs include both global and non-global assay variable NFs, different particular gene comparisons in one assay may have different assay UNFP values. Therefore, in a gene expression analysis assay, one particular gene comparison may be more erroneous and have a higher probability of being associated with an RDM, than another particular gene comparison in the same assay. Such a situation greatly complicates the interpretation of prior art produced N-DGER results. In addition, it greatly complicates the task of correcting or normalizing microarray assay produced particular gene comparison assay RASR values.

Prior art does not determine, or take into consideration during the prior art normalization process for a particular gene comparison, the assay UNFP value for a particular gene comparison. Consequently, it cannot be known whether the assay UNFP for any particular prior art gene comparison is equal to one or not. Therefore, a prior art produced assay N-DGER value for any particular gene comparison cannot be known to be correct with regard to the magnitude of gene expression differences, or the direction of gene regulation change. Thus, absent some knowledge of the assay UNFP value associated with a prior art produced particular gene comparison N-DGER, said N-DGER is essentially uninterpretable with regard to the extent of gene expression activity difference, or the direction of gene regulation change.

To this point, the primary emphasis has been focused on the analysis and interpretation of prior art produced SGDS particular gene RNA transcript comparison N-DGER results obtained from an assay comparison of two cell sample LPN preps. A powerful extension of these microarray analyzes arises from the analysis of the gene expression results of not just one, but many microarray cell sample comparisons, in order to discover common patterns of gene expression in multiple cell samples and pathways of gene expression. Such analyzes are generally termed gene expression data mining (7, 33, 34, 35, 38, 50, 84, 153). A further powerful extension is the use of gene expression results, as well as protein expression and any other pertinent biological or other information to, analyze the biological system. Such an analysis is generally termed a systems biology approach (139). As an example, the prior art often endeavors to identify which individual genes are expressed to similar and different extents in response to some chemical stimulus. To accomplish this, it is necessary to establish a baseline or reference point in order to be able to determine if and when a gene has changed its expression in the treated cell samples. This is generally done by establishing a control or reference cell sample's gene expression profile as the baseline. Then in order to identify the genes in the treated cell sample which have altered their expression, the gene profile of each treated cell sample is compared to that of the reference cell sample, and the N-DGER for each gene of interest is determined. One common data mining method groups together genes which are associated with prior art produced particular gene N-DGERs which have similar quantitative magnitudes and directions of gene expression change. In order for the results of this and other data mining analysis methods, to be known to be valid, and to accurately reflect the pattern of gene expression in the cell sample's examined, the prior art assay N-DGER values used in the data mining analysis must be accurate, interpretable, and intercomparable. As discussed earlier, prior art believes that for each prior art produced particular gene comparison the (assay N-DGER)=(T-DGER), and therefore believes that the prior art produced N-DGER values used in data mining and systems biology analyzes are valid and accurate. Thus, the prior art believes the results of the various data mining and systems biology analyzes are accurate and interpretable. However, since the prior art produced N-DGER values used in these analyzes cannot be known to be correct with regard to the magnitude of gene expression differences, or the direction of gene regulation change, the prior art produced data mining and systems biology results also cannot be known to be correct. Thus, absent some knowledge of the UNFP assay values for the prior art produced particular gene comparison N-DGER values used in the data mining and/or systems biology analyzes, the prior art produced data mining and systems biology analysis results cannot be known to be correct, and are therefore largely uninterpretable.

G. Validity of Assumptions Required for Prior Art Normalization Methods Used to Normalize Prior Art Microarray and Non-Microarray Results.

One or more of the following assumptions must be valid in order for prior art normalization of microarray results to be valid.
(i) Most of the genes, which are active in both compared cell samples, are unregulated (7, 33, 34).
(ii) For those genes, which are regulated in the cell sample comparison, there is a balance between the up and down regulated genes (7, 33, 37, 52, 55, 72, 84, 138).
(iii) In a cell sample comparison the assay results from enough unregulated genes can be identified so that the identified unregulated genes can be used as internal reference genes, from which normalization factors or NFs, can be derived, and then used to accurately normalize other gene comparison results from the same assay (7, 31, 33, 34, 46, 50, 52, 72).
(iv) The genes spotted on the array represent a significantly large random selection of the genes in the compared cell samples (7, 33, 34, 84).
(v) The total RNA content per cell is the same for each compared cell sample (37, 38, 46, 52, 84, 138).
(vi) The total mRNA content per cell is the same for each compared cell sample (37, 38, 46, 52, 84, 138).
(vii) One or more known genes which are active in both compared cell samples are known a priori to be unregulated or to be regulated to a known extent, and such genes serve as internal references from which NFs can be derived, and then used to normalize the other gene comparisons in the gene comparison assay. Such genes are termed housekeeping genes by the prior art (7, 33, 34, 50).

All of these assumptions involve, directly or indirectly, a biological condition which is intrinsic or natural to the cell samples being compared. Assumptions (i) (ii) (v) (vi) and (vii) directly involve the state of the compared cell sample's total RNA or mRNA in the compared cells. Assumption (iii) is dependent on Assumption (i) being valid, and on the ability to identify or describe the assay characteristics of the unregulated genes in the event they are present. Assumption (iv) is known to be valid for high density microarrays, and prior art acknowledges that assumption (iv) is not valid for many low density microarrays. Assumption (vii) is widely regarded as being generally not valid, but is considered by some to be valid in certain limited situations.

The validity of each of these assumptions and the effect of the validity of each of these assumptions on prior art normalized gene comparison results is examined below.

(i) Most Genes which are Active in Both Compared Cell Samples are Unregulated.

Gene regulation occurs in the cell. In the context of this basic biological unit, a gene is either active or inactive in a cell. Relative to other genes in the same cell, or the same gene in another cell, a gene is either unregulated, upregulated, or downregulated. The degree of regulation within a cell is usually expressed in terms of the abundance of the genes mRNA transcripts in the cell, and the abundance is expressed in terms of the number of copies of the particular gene's RNA transcript molecules which are present in a cell. A high abundance gene in a cell is considered to be upregulated relative to a low abundance gene. When a particular gene in a cell has a higher abundance than the same gene in another cell, the higher abundance gene is considered to be upregulated relative to the same gene in another cell which has a lower abundance level. Prior art almost always assumes that the majority of genes which are active in both compared cell samples are not associated with significant differences in gene expression, and are unregulated. That is, the majority of genes in a cell sample comparison have a T-DGER=1, or nearly one. Except for the housekeeping gene normalization method, virtually all other prior art normalization approaches have relied on this key assumption. Current microarray practitioners believe that this is a reasonable assumption, and believe that microarray gene comparison results provide an experimental basis for believing the assumption is reasonable. Outside of the microarray results, which are inconclusive, there is little experimental data, which justifies the assumption. There is, however, solid experimental non-microarray information, which raises a serious concern about the validity of Assumption (i) for many prior art microarray cell sample gene expression comparisons. This is discussed below.

Perhaps the most widely studied living organism is the E. coli bacterial cell. Essentially all aspects of this bacteria have been extensively studied and documented, including the cell morphology, growth characteristics, genetics, biochemistry, and molecular biology. This includes the total RNA, mRNA, DNA, and protein contents per cell for rapidly growing, as well as slowly growing cells (10). It is well known that a rapidly growing E. coli cell contains much more T-RNA and mRNA than a slowly growing cell, and that the actual T-RNA and mRNA contents per cell can be predicted from the growth rate (i.e., doubling time) of the bacterial cells (10). This is also true for other prokaryotes and eukaryotes in general. It is known, for example, that rapidly growing E. coli cells which have a doubling time of 25 minutes contain about 10 fold more T-RNA per cell and mRNA per cell than do E. coli cells which have a doubling time of 57 minutes (10). It is also known that a typical E. coli mRNA has a half-life in the cell of about one minute, and that in a rapidly growing cell about one-half of the newly synthesized RNA is mRNA. It has been reported that for E. coli about 0.04 of the total RNA consists of mRNA (10). Herein, rapidly growing cells, and slowly growing cells are termed RG cells and SG cells.

In the process of converting an SG cell to an RG cell, the amount and number of total RNA and mRNA molecules per cell is increased by 10 fold in the RG cell, relative to the SG cell. Put differently, the amount of both total RNA and mRNA present in the RG cell is upregulated 10 fold, relative to the SG cell. This degree of upregulation in the RG cells suggests that for a microarray comparison of E. coli RG and SG cells, Assumption (i) may not be valid. Assumption (i) specifies that most genes in such a comparison must be unregulated. Whether the 10 fold overall upregulation of total mRNA content in the RG cells causes Assumption (i) to be invalid, depends on the pattern of gene regulation which is associated with converting an SG cell to an RG cell. If most genes which are active in both the RG and SG cells are in fact, unregulated, and only a small fraction of the genes are highly upregulated in the RG, Assumption (i) is valid. However, if most of the genes which are active in both the RG and SG cells are upregulated 10 fold in the RG cells, and only a small fraction of the RG and SG genes which are active in both SG and RG cells are unregulated, then Assumption (i) is invalid. In a situation where it is known that the total mRNA content per cell is significantly greater in one compared cell sample, it is not possible to know whether Assumption (i) is valid or not, absent further knowledge concerning the pattern of gene expression in the compared SG and RG cell samples. As discussed earlier, it is not uncommon for such differences in total mRNA content per cell between different cell samples, even different samples of the same type of cell, to occur in nature. It is well known that total mRNA and/or total mRNA content per cell can: vary significantly, by 2-10 fold or more, in the same type of prokaryotic or eukaryotic cell; vary by 2-25 or possibly more, for different types of cells in the same organism; vary greatly in the same and different types of cells from different organisms; vary significantly with cell size, differentiation, stage of cell growth, ploidy of cells and the disease state of cells. In addition, little is known concerning the effect of a particular physical or chemical treatment on the total RNA and total mRNA contents per cell. It seems clear that many prior art gene expression analyzes have compared cell samples, which had significant differences in total RNA/cell and/or total mRNA/cell. The above-described E. coli SG and RG cell sample comparison illustrates the uncertainty associated with knowing whether Assumption (i) is valid for a cell comparison where a significant difference in the total mRNA/cell is known to occur for the compared cell samples. Adding to this uncertainty is the fact that prior art microarray and non-microarray practice almost never determines, or knows, the total mRNA/cell content or total RNA/cell content of the compared cell samples, and does not consider the effect of the relative amounts of total RNA/cell or total mRNA/cell for the compared cell samples on the normalization method utilized.

In the specific situation when E. coli SG cells and RG cells are compared in a microarray assay it is possible to determine whether Assumption (i) is valid because: (a) The relative amounts of total RNA/cell and total mRNA/cell are known for SG and RG cells with known doubling times; (b) A global E. coli microarray measured gene expression profile for the comparison of SG and RG cells with doubling times of 57 minutes and 25 minutes is available in the literature (143), and the assay raw results are available at (www.ou.edu/microarray). Arrays containing all 4,290 E. coli genes were used to generate a gene expression profile comparison of SG E. Coli cells in minimal glucose media which had a doubling time of 57 minutes, and RG E. coli cells grown in rich media with a doubling time of 25 minutes. The comparison was done with radioactive labeled cDNA. The microarray gene comparison results were normalized using a version of the prior art TIN method, where each individual gene spot intensity was expressed as a percentage of the total of all of the gene spot intensities on an array. This then, allowed for the direct comparison of the results from the compared arrays. A normalized expression ratio was determined for each of the genes in the comparison, which were active in both the SG and RG cell. A normalized (SG/RG) expression ratio of greater than 2.5 or less than 0.4 was considered to reflect a statistically significant change in gene expression. By this standard the great majority, about 2,846 genes, of the about 3,190 genes which were measured active in both SG and RG cells, do not differ significantly in gene expression extent. This number and following numbers were obtained from analysis of the raw assay data from the web site www.ou.edu/microarray, provided by Dr. T. Conway. These genes are therefore, considered to be unregulated. This study found that 3,496 genes were active in SG cells, and 3,284 genes were active in RG cells. In addition to the about 2,846 unregulated genes, 225 genes which were active in both SG and RG cells were significantly upregulated in SG cells, and 119 genes which were active in both SG and RG cells were significantly upregulated in RG cells. For the 225 genes which are active in both RG and SG cells, and which are upregulated in the SG cells, the (SG/RG) expression levels range from just over 2.5 to 74, and only 6 of these upregulated genes have ratios of 10 or more. It appears that the total number of upregulated SG cell mRNA molecules is greater than the total number of RG cell upregulated mRNA molecules. For the 119 genes which are active in both the SG and RG cells and which are upregulated in the RG cells, the expression levels range from 2.5 to 10 fold, relative to SG cells. None of these 119 RG cell genes are upregulated over 10 fold. In addition, about 96 genes are active in the RG cell and inactive in the SG cells and are therefore upregulated in the RG cells, while about 307 genes were active in the SG cells and inactive in the RG cells, and are therefore upregulated in the SG cells. Table 30 presents a summary of these results. Note that the results originate in part from the TAO et al., published report, and part from the raw data from the website www.ou.edu/microarray (143).

The results of this prior art microarray gene expression comparison analysis were normalized using a standard prior art normalization method. These results indicate that 2,846 genes, the great majority of the genes which are active in both the RG and SG cells have been measured to be unregulated. In this context, it appears that the generally believed assumption that most of the genes, which are active in both compared cell samples, are unregulated, is true. Many prior art microarray gene expression analyzes have generated similar results and these results have strengthened the widespread belief in the general validity of Assumption (i).

TABLE 30
Gene Activity Budget For the E. coli RG Cell and SG Cell Comparison

| | |
|---|---|
| | Fraction of Total |
| | RG Assay Signal |
| Activity of Genes In | Associated |

| Number of Genes | | RG Cells | SG Cells | with Genes |
|---|---|---|---|---|
| 3,190 | Genes | + | + | — |
| 96 | Genes | + | — | 0.002-0.004 |
| 307 | Genes | — | + | — |
| 697 | Genes | — | — | — |

^{(a)}119 genes active in both SG and RG cells and upregulated in RG cells, and 225 genes are active in both SG and RG cells and are upregulated in SG cells.
^{(b)}Total number unregulated genes = (3,190 − 119 − 225) = 2,846 genes.
^{(c)}Total signal on SG array = 1.69 × 10⁷ signal units. Total signal on RG array = 1.66 × 10⁷ signal units.
^{(d)}Criterion for active gene ≧500 signal units (˜0.003% of total). For active gene ≦˜499 signal units.

Interestingly, the results of this SG and RG gene activity comparison do not identify a small group of RG genes which are responsible for the bulk of the 10 fold increase in mRNA content per cell in the RG cells, relative to the SG cells. Only 119 genes which are active in both SG and RG cells are upregulated in the RG cells, and the degree of upregulation, relative to the SG cells, ranges from 2.5-10 fold. The average degree of upregulation for these 119 RG genes is roughly 4 fold. This degree of cell upregulation for these 119 genes does not account for anywhere nearly enough RG mRNA molecules to account for the 10 fold greater mRNA content/cell present in RG cells. The only other possible source of the 10 fold increase in the RG cell total mRNA content/cell are the 96 upregulated RG genes which are active in the RG cells and not active in SG cells. As indicated in Table 30 these genes account for just 0.2-0.4% of the total assay signal for RG cells. In order for these 96 genes to account for all of the 10 fold increase in the mRNA/cell content of RG cells, the assay signal associated with these genes would have to constitute about 90% of the total RG cell normalized assay signal. This indicates that the bulk of the 10 fold greater mRNA/cell content in the RG is due to a general about 10 fold upregulation of many different genes, and that assumption (i) is invalid.

It is useful to illustrate this discussion in terms of the number of mRNA molecules per cell which are typically present in SG and RG cells. Table 31 presents the total RNA and total mRNA contents per cell for SG and RG E. coli cells (10). A SG cell contains 1,550 mRNA molecules, while an RG cell contains 15,500 individual mRNA molecules. Each RG cell then, contains about 14,000 more mRNA molecules than does each SG cell.

TABLE 31
RNA Content of SG and RG E. coli Cells

| | | | | ^{(b)}Number of | |
|---|---|---|---|---|---|
| | | Femtograms/Cell | | Average | Number of |
| | (Minutes) | (fg) | | Gene | Active |
| | Doubling | Total | | Sized mRNA | Genes in |
| Growth Media | Time | RNA | ^{(a)}mRNA | Per Cell | Cell |
| Minimal (SG) | 57 | 20 | 0.8 | 1,550 | 3,496 |
| Rich (RG) | 25 | 200 | 8 | 15,500 | 3,284 |

^{(a)}Assumes 0.04 of total RNA is mRNA.
^{(b)}Assumes average gene mRNA is about 1,040 nucleotides long.
(c) Estimated from data in.

As discussed above, the genes responsible for the presence of the extra 14,000 molecules in the RG cells cannot be identified in prior art normalized results of an E. Coli microarray gene expression comparison of RG and SG cells. In addition, these same results indicate that the use of Assumption (i) for the normalization of the raw assay results is valid. Both of these issues will be further discussed below.

An earlier section discussed the effect of the use of the EA Rule, and the existence of natural differences in the total RNA/cell and total mRNA/cell for different cell and tissue types, on prior art microarray and non-microarray gene expression results. In the above-described microarray comparison of SG and RG cells: (a) The EA Rule was practiced by comparing equal masses of SG and RG T-RNA, and; (b) RG cells contained 10 fold more T-RNA and T-mRNA than SG cells. In the said microarray comparison of SG and RG cells a prior art version of TIN was used for normalizing the gene comparison results (143), and no consideration was given to normalizing the assay gene expression results for differences in the number of SG and RG cells compared in the assay. In other words, the assay SCR was not determined, and the assay gene expression ratio results were not normalized for the SCR. Since: the T-RNA content/cell of the RG cells with a doubling time of 25 minutes is known to be 10 fold greater than the T-RNA/cell for SG cells with a doubling time of 57 minutes, and equal masses of T-RNA from SG and RG cells were compared in the assay, then the (SG/RG) SCR value=10 for the assay. As discussed earlier, the measured gene expression ratio for a gene is divided by the SCR in order to normalize the particular gene expression ratio for the SCR. This means that each assay gene expression ratio is divided by 10 in order to obtain an SCR normalized gene expression ratio for each particular gene in the assay. Table 32A presents a summary of the assay prior art normalized gene expression ratios, which have been further normalized with the SCR. As indicated in Table 32A, before SCR normalization the majority of genes, which were active in both SG and RG cells were measured to be unregulated. However, after SCR normalization only about 30 of the genes which are active in both SG and RG cells, are unregulated, and none of these genes were considered to be unregulated before SCR normalization. The same criterion for statistically significant differences in expression levels is used for before and after SCR normalization. That is, that a (SG/RG) ratio greater than 2.5 or less than 0.4, indicates a significant difference in expression levels.

TABLE 32A
SCR Normalization of E. coli Gene Expression Results

| | | Range of | Range of SCR | Overall Interpretation of Gene Regulation For | |
|---|---|---|---|---|---|
| | | (SG/RG) | Normalized | Gene Expression Results | |
| | Number of | Expression | Gene | Prior Art | |
| Gene | Genes in | Ratios for Genes | Expression | Before SCR | |
| Category | Category | Before SCR | Ratios | Norm. | ^{(a)}After SCR Norm. |
| Unregulated | 2,846 | 0.4 to 2.51 | 0.04 to 0.251 | All 2,846 Genes | All 2,846 Genes Upregulated By 4-25 |
| | | (1/2.51) to (2.51/1) | (1/25) to (1/4) | Unregulated | Fold |
| Genes Active | 225 | 2.51 to 74 | 0.251 to 7.4 | 225 Genes | 6 Upregulated in SG Cells |
| in SG and RG | | | (1/4) to (7.4/1) | Upregulated in | 186 Upregulating in RG Cells |
| Cells and | | | | SG Cells | 33 Unregulated |
| Upregulated | | | | | |
| in SG Cells | | | | | |
| Genes Active | 119 | 0.4-0.1 | 0.04-0.01 | Genes | All 119 Genes Upregulated 25.1 to |
| in SG and RG | | (1/2.51 to 1/10) | (1/25 to 1/100) | Upregulated 2.51 | 100 Fold in RG Cells |
| Cells and | | | | to 10 Fold in RG | |
| Upregulated | | | | Cells | |
| in RG Cells | | | | | |

^{(a)}Assumes same criterion for expression level significance as in TAO et al., publication.

The prior art interpretation of the prior art normalized (SG/RG) gene expression ratios indicates that the great majority of the genes, about 2,846, in this gene comparison assay, are unregulated. In this prior art context, Assumption (i) is clearly a valid assumption for normalization. After SCR normalization none of the 2,846 genes interpreted by the prior art as being unregulated, are unregulated. After SCR normalization, only about 30 genes fall in the unregulated category, and all 30 of these genes were identified by the prior art as being upregulated in the SG cells. These observations dramatically illustrate the difficulty in identifying the unregulated genes in prior art microarray gene expression comparison normalized assay results, in a situation where the compared cell samples have significantly different total RNA/cell and total mRNA/cell contents.

In the context of the SCR normalized results for the E. coli SG and RG cell sample microarray gene expression comparison, Assumption (i) is clearly not a valid assumption. This definite conclusion could be determined because: (a) The total RNA/cell and total mRNA/cell contents are known for both E. Coli SG and RG cells with known doubling times and these doubling times were reported in the TAO et al., publication; (b) All of the E. coli genes were represented on the microarray; (c) Enough could be discerned from the available TAO et al., microarray results so that a rough pattern of gene regulation in the RG cells and SG cells could be determined; (d) The effect of the SCR on the assay results was considered; (e) The EA Rule was utilized in the assay; (f) TAO et al., provided excellent and relatively (compared to most microarray reports) complete and pertinent information in their report.

It was discussed earlier that a significant difference in mRNA/cell content for compared cell samples could occur in several ways. One way is for most of the genes in the comparison to be unregulated, while a subset of genes in the cell sample comparison are highly upregulated in the cells, which have the greater total mRNA/cell content. In this case, Assumption (i) would be valid for the cell comparison. A second way is to upregulate all or a large fraction of the active genes for the compared cells, which have the greater total mRNA/cell content. In this second case, the majority of active genes would not be unregulated and Assumption (i) would be invalid. Situations intermediate between these two extremes may also occur. In these intermediate situations it will be generally more difficult to determine the validity of Assumption (i). In a microarray assay situation where it is known that the total mRNA/cell content of one compared cell sample is greater than the other, it is not currently possible to know which pattern of gene regulation exists for any particular microarray cell sample comparison, without experimentally determining the true pattern of gene regulation in the compared cell samples. In order to determine the true pattern of gene regulation the total RNA/cell and/or total mRNA/cell contents of the compared cells must be known and taken into consideration in the normalization process. This was done for the above-described microarray comparison of E. coli SG and RG cells. The results indicate that the greater mRNA/cell content for RG cells is due to an overall roughly uniform upregulation of the large majority of genes which are active in the RG cells, and that only a small percentage of the RG active genes were actually unregulated. The consequence of the existence of this gene regulation pattern is that Assumption (i) can be known to be not valid for this microarray comparison. This result has serious implications for prior art microarray cell sample gene expression comparisons in general. This is discussed below.

The above-described E. coli microarray assay involved the comparison of E. coli cells at different growth or cell cycle stages. It is well known for both prokaryotic and eukaryotic cells, that the total RNA/cell and total mRNA/cell contents of the same type of cell generally differs significantly for cells at different growth rates and stages of the cell cycle. These differences can range from 2-10 fold or more. It is not uncommon for prior art microarray practice to compare cells of the same type which are at different growth or cell cycle stages. If the gene regulation pattern associated with the cell cycle differences in total mRNA/cell content, generally involves a uniform or roughly uniform upregulation of most of the active genes in one cell sample, then Assumption (i) is generally not valid for prior art microarray assays associated with cell samples which have cell cycle or growth stage differences in total RNA/cell and/or total mRNA/cell contents. Cell cycle or growth stage differences in cells can be induced by multiple factors including nutrients, hormones, chemicals, drugs, physical treatment, and other factors. Little is known regarding the effect of most of these factors on the cell cycle or growth stage of particular cell types or cells in general. It is clear that prior art microarray and non-microarray gene expression analysis practice has often compared cell samples possessing significantly different cell cycle or growth stage related total RNA/cell and/or total mRNA/cell contents. However, with few exceptions, it is not possible to identify such particular prior art cell comparisons. Prior art only rarely determines or knows whether cell cycle or growth stage differences are present in the compared cell samples. Further, prior art only rarely determines or knows whether the total RNA/cell and total mRNA/cell contents of the compared cell samples differ, and does not consider the total RNA/cell or total mRNA/cell contents, or the SCR of the compared cell samples during the microarray data analysis process. As a result of all this, the general extent of occurrence of cell cycle or growth stage related differences in the total RNA/cell and/or total mRNA/cell contents of the compared cell samples is not known, even though such occurrences are highly likely to have occurred often. As a consequence, it cannot be known whether Assumption (i) is valid or not for the vast majority of particular microarray cell comparisons, although it is highly likely that Assumption (i) is invalid for many of these prior art assays.

It is well known that the total RNA/cell and total mRNA/cell contents can vary greatly for the same cell type at different stages of differentiation, and for different cell types in the same organism. Such differences also occur between the same and different cell types in different organisms. Such differences in total RNA/cell and total mRNA/cell content can range from 2 to 25 fold or more, depending on the specific cell sample comparison. At present the pattern or patterns of regulation which occurs when differences in total RNA/cell and/or total mRNA/cell are associated with different stages of differentiation, is not known. Different patterns of regulation may well be associated with different cell types or tissue types. A particular tissue may be associated with more than one pattern of regulation. It is not uncommon for prior art microarray practice to compare different types of cells or tissues. If the gene regulation pattern for these different type of cell comparisons involves a uniform or roughly uniform upregulation of most or many of the active genes in the high mRNA/cell content cell sample, then Assumption (i) is not valid for these cell comparisons. Differences in the differentiated state can be induced by multiple factors including nutrients, hormones, chemicals, drugs, physical treatment, and other factors. Little is known regarding the effect of many of these factors on the differentiation mechanism and the total mRNA/cell and/or total mRNA/cell content of different differentiated cells and tissues. It is clear that prior art microarray and non-microarray gene expression analysis practice has often compared cell samples, which possessed significantly different differentiation state related total RNA/cell and total mRNA/cell contents. It is possible, because of limited prior art knowledge concerning the total RNA/cell and/or total mRNA/cell contents of certain differentiated cell or tissue types, to identify particular prior art microarray cell sample comparisons where the compared cell samples have significantly different total RNA/cell and/or total mRNA/cell contents. However, knowledge concerning the upregulation patterns for the cell sample with the greater total mRNA/cell content is not available. Therefore, it is not possible to know whether Assumption (i) is valid for these comparisons or not. Note that tumor or cancer cells are here considered to be different states of differentiation, and the above discussion applies directly to them. In a similar vein, aspects of the above discussions on cell cycle and differentiation stage effects on the total RNA/cell and total mRNA/cell content of cells applies directly to the total RNA/cell and total mRNA/cell content of diseased or otherwise damaged cells of all kinds, and to the uncertainty of knowing whether Assumption (i) is valid for microarray cell sample comparisons involving one or more diseased or damaged cell samples. Note that the state of the total RNA/cell and/or the total mRNA/cell content for any cell at any time is influenced by both the cell cycle or growth stage of the cell and its differentiation and treatment state.

It is also known that the total RNA/cell and/or total mRNA/cell content of cells can vary significantly due to cell size and ploidy. Generally the larger the cell size and the higher the ploidy of a cell, the greater the total RNA/cell content and it is likely that the total mRNA/cell content is also greater. Ploidy changes are observed in many cancer cells and virtually all continuous cell cultures are aneupolid. It is not known how such changes affect the total RNA/cell and/or total mRNA/cell contents of continuous cell cultures. Overall, little knowledge exists concerning the effect of cell size or ploidy changes on the total RNA/cell content, and even less on the total mRNA/cell content. It is clear that prior art microarray and non-microarray practice has often compared cell samples, which differ in cell size and ploidy. However, the effect of such differences on the validity of Assumption (i) cannot be known without further knowledge. Note that the state of a cells total RNA/cell content and/or total mRNA/cell content at any one time is influenced by the cells cell cycle or growth stage, its state of differentiation and treatment, its cell size, and its ploidy. The ploidy of the cell may influence all of the other factors.

As discussed above, the conversion of E. coli SG cells to RG cells is associated with a large general upregulation in the RG cells of the majority of genes which are active in both SG and RG cells. This raises the possibility that a similar general gene upregulation occurs for the conversion of all prokaryotic and eukaryotic cells from SG cells to RG cells, and that a general gene downregulation occurs for these cell types when a cell converts from RG to SG. If such a general gene regulation pattern is associated with the cell cycle and growth stages of all prokaryote and eukaryote cells, then Assumption (i) would be invalid for any microarray assay associated with significant differences in total RNA/cell and total mRNA/cell content which are related to cell cycle or growth stage differences in the compared cell samples. In this context, it is reasonable to believe that many prior art prokaryote and eukaryote microarray cell sample gene comparisons cannot validly assume Assumption (i). However, many prior art microarray practitioners believe that evidence from prior art microarray gene comparisons validates Assumption (i). This is believed because for many prior art microarray assays, the measured normalized expression levels for the majority of the particular gene comparisons in the assay, are not statistically different, and are therefore considered to be unregulated, or nearly so. The above-described microarray cell comparison of E. coli SG and RG cells is an example of a prior art microarray gene comparison assay for which such a conclusion was reached. TAO et al., concluded from their prior art measured and normalized SG and RG expression levels, that the majority of genes (about 2,846 genes) active in both the SG and RG cells did not differ significantly in expression levels between growth conditions, and therefore were unregulated, or nearly so. As discussed above, after further SCR normalization of the TAO et al., gene expression results, only about 30 genes do not differ significantly, and are therefore unregulated. Such a situation, where the majority of compared genes are prior art normalized and measured to be unregulated and the SCR normalized results indicate that very few of the compared genes are unregulated, is the result of the interaction of the practice of the EA Rule, the similar increases in both T-RNA/cell and total mRNA/cell content of the RG cells, and the regulation pattern which exists for the SG and RG cell comparison. Because the EA Rule is practiced for the assay, the relative number of SG cells in the hybridization solution is 10 fold higher than the number of RG cells, because the T-RNA/cell content of RG cells is 10× that of SG cells. This results in the relative (SG/RG) concentration ratio of each particular genes mRNA in the hybridization solution being 10× higher, than the relative (SG/RG) ratio of each particular gene which is present in the SG and RG cells. Thus, for any particular gene mRNA in the assay which has a relative (SG/RG) cellular abundance ratio of 0.1 or nearly 0.1, the hybridization solution relative (SG/RG) concentration ratio will be 1 or nearly 1. As a consequence, the prior art measured and normalized (SG/RG) expression level ratio, will be equal to 1 or nearly 1. Limited information indicates that both prokaryotic and eukaryotic cells exhibit similar general characteristics with regard to increases of total RNA/cell and total mRNA/cell contents of rapidly growing cells relative to slowly growing cells. The general pattern is that both total RNA/cell and total mRNA/cell contents increase by substantial but not always equal amounts. As an example, as described earlier, mouse cell culture rapidly growing 3T3 cells contain 4× more total RNA/cell and 6× more total mRNA/cell, relative to slowly growing 3T3 cells. Mouse cultured growing 3T6 cells show a similar pattern, but the degree of increase is less (1, 14).

The above discussion indicates that a combination of microarray assay practice, biological characteristics intrinsic to the compared cell sample, and an inadequate prior art normalization procedure, can result in the prior art misidentification of many genes as unregulated, when they are in fact significantly regulated. This almost certainly has occurred in the prior art microarray practice and has contributed to the prior art view that for most microarray cell comparison assays the majority of genes which are active in both cell samples are unregulated. It should be noted that such situations cannot be validly normalized for by any prior art normalization practice methods involving TIN or local TIN methods, or scatterplot or ranking methods. The housekeeping gene approach would properly correct such a situation, but prior art consensus is that housekeeping genes with the appropriate characteristics are not available.

The gene regulation pattern where large numbers of genes in a cell type or tissue are up or down regulated together, could also be associated with other factors than the cell cycle or growth stage. For example, such a general gene regulation pattern may be associated with: normal differentiation of cells, as well as abnormal differentiation of cells to form cancers, tumors, or some other disease state; size and ploidy changes in cells; and various drug, chemical, and physical treatment of cells. Alternatively, each of these different situations may be associated with a different pattern of global and non-global regulation.

The above discussions indicate that Assumption (i) is not valid for certain prior art microarray and non-microarray gene expression analyzes, and may not be valid for many prior art microarray assay cell comparisons. Further, with few exceptions, it is not possible to know whether Assumption (i) is valid for any particular prior art microarray or non-microarray cell comparison. With the proper information, it is possible to know when Assumption (i) is valid. However, that information is not available for prior art microarray and non-microarray assays.

(ii) In the Microarray Cell Sample Comparison there is a Balance Between Up and Down Regulated Genes.

The just discussed section on the validity of Assumption (i) is directly pertinent to the validity of Assumption (ii). Clearly for those microarray assay situations where a significant difference in the total mRNA/cell content is present for the compared cell samples, a significant degree of upregulation has occurred in one compared cell sample and Assumption (ii) is not valid. This is true whether the increased mRNA/cell content of the cell sample is due to a general upregulation of all or most active genes, or to the upregulation of one or a relatively small number of genes. As discussed, it is known that prokaryotic or eukaryotic cells of the same type have 2-10 fold or more, differences in total RNA/cell and total mRNA/cell contents. In addition, different normal and abnormal cell types in one organism can have roughly 2-25 fold differences in total RNA/cell and total mRNA/cell contents. As discussed, differences in cell size, cell ploidy, the disease state of the cell, and exposure to drugs, chemicals, physical treatment, and other factors, may result in a greater total mRNA/cell content for one cell sample relative to a compared cell sample. It is clear that prior art microarray and non-microarray practice has often compared cell samples, which differ in total RNA/cell and total mRNA/cell content. Assumption (ii) is not valid for such microarray assays.

For the large majority of prior art microarray and non-microarray cell comparisons, it is not known whether the total mRNA/cell content of one cell sample was greater than the compared cell sample or not. Therefore, it cannot be known whether Assumption (ii) is valid for these assays or not, since prior art does not determine the total RNA/cell and/or total mRNA/cell contents of the compared cell samples. Therefore, with certain exceptions, it is not possible to know whether Assumption (ii) is valid for any particular microarray or non-microarray cell sample comparison. With the proper information this could be determined. However, the information is not available.

It should be noted that in certain microarray assay situations, even when the up and down regulated genes are balanced in the compared cell samples, an erroneous normalization factor can result. These certain conditions involve the pattern of up and down regulation, which exists in the compared cells, and the just detectable mRNA abundance level of the assay. The first requirement is an up and down regulation pattern where in one sample a relatively small number of genes mRNA is upregulated to high abundance, and in the other cell sample a larger number of different low abundance genes are upregulated just 2-3 fold, and the total amount of up and down regulated mRNA is the same for both compared cell samples. The second requirement is that the microarray assay just detectable mRNA abundance level allows the detection of all of the highly upregulated mRNA from one sample, and only a fraction of the low abundance upregulated mRNA from the other sample. Such microarray assay just detectable conditions are common for mammalian cell sample comparisons.

(iii) Assay Results Associated with Unregulated Particular Genes can be Identified and Used to Generate One or More Normalization Factors (NF) which Will Correctly Normalize all Other Assay Particular Gene Comparison Results.

Assumption (iii) then, requires the following. (a) A significant number of assay results associated with unregulated genes must be identified, and distinguished, from regulated gene assay results. (b) The NF or NFs generated from the identified unregulated gene results must accurately normalize other assay results so that the normalized gene expression level ratios are biologically correct. That is, so that the normalized assay result ratio (NASR)=(T-DGER), for each particular gene comparison in the assay.

The prior art global and local TIN based methods of normalization require that Assumption (i) be valid, but do not require the identification of the assay results associated with a significant number of unregulated genes, and therefore do not require the validity of Assumption (iii). In contrast, the prior art methods of normalization involving global and local regression analysis, scatterplots, ranking, and other methods, require being able to identify a significant number of assay results associated with unregulated genes, and therefore require the validity of both Assumption (i) and (iii.)

Technically, certain prior art normalization methods do not identify specific unregulated genes in the assay, but assume that the center or mean of the distribution of unregulated gene comparison assay RASR results can be identified, quantified, and used for normalization. For the purposes of this discussion on the validity of Assumption (iii), this is equivalent to identifying specific unregulated genes. For simplicity this discussion will be in terms of correctly identifying specific unregulated genes. This discussion will be directly applicable to identifying the center or mean of the distribution of unregulated gene comparison assay RASR values.

The discussion on the validity of Assumption (i) is directly pertinent to the validity of Assumption (iii). Discussion (i) concluded that: Assumption (i) is not valid for some prior art microarray assay cell comparisons; Assumption (i) may not be valid for many prior art microarray cell comparisons, and; it is not possible to know whether Assumption (i) is valid or not for most prior art microarray assay cell comparisons. Clearly, if Assumption (i) is not valid, then the identification of the unregulated gene results is problematic, and Assumption (iii) is not valid. The prior art view is that unless a majority of the genes which are active in both compared cell samples are unregulated, identifying the unregulated gene assay results, and distinguishing them from regulated gene results, is problematic.

The Assumption (i) discussion described a prior art microarray assay cell comparison of SG and RG E. coli cells which showed that a combination of, common microarray assay practice, biological characteristics intrinsic to the compared cell samples, and an incomplete prior art normalization procedure, resulted in the following. The misidentification of the majority of the genes in the assay as being unregulated, when in reality those genes were all regulated to a significant degree, and the misidentification of the actual unregulated genes, as regulated. This prior art microarray example suggests that the conditions, which cause the misidentification of regulated and unregulated gene results, occurs often in prior art microarray practice. The reasons for this are discussed in the Assumption (i) section. For most particular prior art microarray assay cell comparisons, it is not possible to know whether the particular gene comparison assay results identified as being associated with unregulated genes, are actually associated with unregulated genes, or not. Similarly, the actual regulatory status of certain assay results, which are identified as being regulated, is also unknowable. With the proper information it is possible to determine the true regulatory status of a gene assay result. However, prior art does not determine the information required to accomplish this.

The following discussion on the validity of Assumption (iii) will assume that Assumption (i) is valid. Prior art practices and believes that when Assumption (i) and (iii) are valid: it is possible to identify assay results associated with unregulated genes, and distinguish them from regulated gene assay results; and then use the identified unregulated gene results to generate one or more assay normalization factors or NFs; and then use the one or more assay NFs to normalize all other assay results to produce biologically correct NASR values for each particular gene comparison.

Prior art generally believes that the microarray and non-microarray assay result for each particular gene comparison must be normalized in order to produce biologically correct results. As discussed earlier, an assay result for a particular gene comparison includes the raw assay signal, or RAS, which is associated with each gene in each cell sample, and the RAS ratio, or RASR, which is the ratio of the RAS values for a particular gene comparison. The normalized RAS is the NAS, and the normalized RASR is the NASR. Prior art believes that such normalization is necessary because of the existence of prior art known assay variables, which cause the assay RASR value for a particular gene comparison to deviate away from the biologically correct value. The aim of the normalization process is to correct the assay RAS and/or RASR results, for all pertinent assay variables which cause the assay RASR value for a particular gene comparison to deviate from the biologically correct T-DGER value.

Assay variables, which are known and considered in the prior art normalization process, have been discussed earlier. Prior art belief and practice is that, when a particular gene comparison assay RASR result is normalized with the prior art known and considered assay variables, the resulting assay (NASR)=(T-DGER). Such prior art belief is valid only if all pertinent microarray or non-microarray assay variables have been taken into consideration in the prior art normalization process. Since prior art believes and practices that after prior art normalization the assay (NASR)=(T-DGER), then prior art believes that all of the pertinent assay variables are known and considered in the prior art normalization process. Also discussed earlier were multiple assay variables which can cause the assay RASR to deviate significantly from the T-DGER, and which are not considered in the prior art microarray and non-microarray normalization process.

When Assumption (i) is valid, Assumption (iii) is invalid if one of the following circumstances occurs. (a) It is not possible to identify the microarray assay results, which are associated with unregulated genes, and distinguish the unregulated gene results from the regulated gene assay results. (b) Normalization of assay results with the one or more NFs derived from the identified unregulated assay results, does not produce biologically correct mRNA expression level ratios for each particular gene comparison in the assay. The following discussion pertains to factors, which can cause (a) or (b) to occur.

Prior art believes and practices that, since the majority of genes which are active in both cell samples are unregulated, the assay results associated with these unregulated genes should have assay values which are similar, and the similarities can be used to identify and distinguish unregulated gene assay results from regulated gene assay results. In essence, this approach identifies a significant number of genes which have similar assay results, and because it is believed that the majority of genes active in both cell samples are unregulated, these similar results are believed to be associated with unregulated genes and T-DGER=1 values. The approach assumes that significantly regulated gene results will not share these similar result characteristics.

Prior art uses global and local regression analysis, scatterplots, ranking, and other methods to identify and distinguish a significant number of assay results which are associated with unregulated genes which are active in both compared cell samples. Prior art then uses these unregulated gene assay results to determine: a single global normalization factor (NF) which is used to normalize all other gene comparison assay results on the array, or; multiple “local” NFs, each of which is applicable to only a subset of the assay results. Prior art believes that Assumption (i) and (iii) must be valid in order to obtain valid global or “local” NFs.

Prior art microarray normalization practice uses the identified unregulated gene assay results to determine either a single global NF value, or multiple local NF values. The assay global NF value for normalizing the assay measured gene comparison assay RASR value for a particular gene comparison, is equal to, (the identified unregulated gene RASR)÷(the T-DGER of the unregulated genes). Since the unregulated gene T-DGER=1, then (the global NF value)=(the unregulated gene assay RASR). This single prior art global NF value is then used to normalize each particular gene comparison RASR value in the assay to generate an assay NASR value for each gene comparison. This normalization is accomplished by dividing a particular gene comparison assay RASR by the pertinent assay variable NF value. This will yield a NASR value for the particular gene comparison. Such NASR value will be completely normalized and equal to the T-DGER for the gene comparison, when the assay RASR value has been normalized with all pertinent assay variable NF values. Note that the normalization process can also be done on the assay RAS values using assay variable NF values, which are in a different form.

Prior art often believes and practices that a prior art determined global NF is a true global NF and that normalization of each particular gene comparison RASR with the global NF, will produce a NASR value for the gene comparison which is biologically correct. A prior art determined global NF value for a particular microarray assay virtually always represents more than one particular assay variable. The prior art determined global NF is almost always a composite of the products of multiple different pertinent assay variable NFs. Thus, a prior art practice global NF typically is believed to normalize each particular gene comparison RASR for multiple different assay variables. Prior art believes and practices that the assay variables associated with differences in amounts of compared cell sample RNA, differences in labeling and detection of mRNA LPN molecules, and hybridization kinetic differences associated with the assay hybridization solution composition, are normalized for by prior art global assay NFs.

Local assay variable NFs are associated with non-global assay variables. A non-global assay variable is a single assay variable, which can affect different gene comparisons in the same assay to a different quantitative extent. A particular non-global assay variable local NF value represents the NF value, which can be used to normalize a particular subset of assay gene comparisons for the non-global assay variable. In essence, a particular assay variable's local NF value for a particular subset of regulated or unregulated gene comparisons in an assay, is equal to, (the identified unregulated gene assay RASR value associated with the particular subset of gene comparisons)÷(1). This prior art determined particular assay variable's single unregulated gene NF value, is used to normalize each particular gene comparison RASR value in a particular subset population, for the particular unregulated gene assay variable. Prior art often identifies and normalizes for three different non-global assay variables, which require normalization with local NF values. These are the spatial, intensity, and print tip assay variables. Thus, each particular gene comparison in an assay for which the three assay variables are pertinent, is normalized with local assay variable NF values for three different assay variables. Prior art believes and practices that when a particular gene comparison RASR is properly normalized for prior art known and considered global and local assay variables, the NASR produced is biologically correct.

For a particular cell sample gene expression comparison there is only one assay value for each particular global assay variable NF, and that assay NF value can be applied to each particular gene comparison RASR value in the assay. There can be, and usually are, more than one pertinent global assay variable in each assay, and each different global NF can have a different quantitative value. For a single microarray assay there can be and usually are, multiple different pertinent non-global assay variables associated with the assay. Each of the particular non-global assay variables can be associated with multiple assay non-global NF values, and each particular non-global NF value is associated with a different subset of gene comparisons in the same assay.

Prior art believes that microarray and non-microarray assay results for each particular gene comparison must be normalized in order to produce biologically correct assay measured gene expression ratios. Prior art believes that such normalization is necessary because of the existence of global and non-global assay variables in the assay, which causes the measured assay RASR values for particular gene comparisons to deviate from the biological correct values. A typical prior art microarray and non-microarray gene comparison assay is virtually always associated with one or more global assay variables, and one or more non-global assay variables, and each non-global assay variable is almost always associated with multiple different NF values. For any particular gene comparison in an assay, the aggregate effect of these assay associated global and non-global assay variable NFs, can cause the assay RASR value to deviate from the T-DGER value for that particular gene comparison. In such a situation, the separate NF values for each global and non-global assay variable can interact to cause the deviation for a particular gene comparison in the assay, to be small, large, or non-existent. In order to know whether the prior art normalization process is valid for each particular gene comparison, it is necessary to somehow obtain an accurate measure for aggregate effect of all of the pertinent global and non-global assay variables on the particular gene comparison's assay RASR value. It is unlikely that this can be obtained unless all of the assay associated global and non-global assay variables can be identified, and the method for obtaining a measure of each variables NF is valid. Note that different prior art assays can, and usually are, be associated with different assay variables.

As discussed earlier, multiple global and non-global assay variables exist, which are not identified and considered in prior art normalization. All of these previously unconsidered assay variables can cause an assay RASR value for a particular gene comparison to deviate significantly from biological correctness. The existence of such multiple previously unconsidered assay variables suggests that many prior art normalized assay NASR values are incompletely normalized, and therefore biologically incorrect. The impact of these prior art unconsidered global and non-global assay variables on the validity of Assumption (iii), is discussed below.

For an unregulated gene, the quantitative assay RASR value is influenced by unwanted assay signal associated with multiple global assay variables, unwanted assay signal associated with multiple non-global assay variables, and wanted assay signal concerning the true difference in gene expression for the unregulated gene, which exists in the compared cell samples. In order to determine the wanted assay signal value for the unregulated gene, it is necessary to adjust or normalize the unregulated genes assay RASR for all significant global and non-global assay biases present in the gene's assay measured RASR.

In the absence of pertinent non-global assay variables, all unregulated genes in the assay would have essentially the same assay RASR value. Each global assay variable pertinent to the assay will affect each unregulated gene RASR to the same extent. Thus, even when one or more pertinent global assay variables are associated with each unregulated gene assay RASR value, all unregulated gene RASR values will be the same, or nearly the same, in the absence of non-global assay variables. In addition, each significantly regulated gene assay RASR value in the assay will be significantly different than the unregulated genes RASR value. This situation is optimum for the identification of unregulated gene RASR results, and distinguishing these results from regulated gene assay results.

Any assay factor or factors which reduces the similarity of different unregulated gene's RASR quantitative assay values, will complicate the identification of such unregulated genes on the basis of assay RASR value similarity. In addition, it will be more difficult to distinguish between assay RASR values from unregulated and regulated genes on the basis of RASR value differences. If such factors reduce the similarity between the individual unregulated genes enough, it will not be possible to identify the different unregulated gene assay RASR values on the basis of their similarity. In addition, it will not be possible to distinguish unregulated gene assay RASR values from regulated gene assay RASR values, on the basis of their differences. As discussed, virtually all prior art microarray assay particular gene comparison RASR values, including those for unregulated genes, are associated with multiple non-global variables. These non-global variables include the prior art considered spatial, intensity, print tip, and print plate NFs, as well as the prior art UNFs MLDR, PL-HKR, PS-HKR, PSAR, PSSR, SBNR, SSAR, and LLSR. Each of these unconsidered non-global assay variables can cause the assay RASR value for a particular unregulated gene comparison, or regulated gene comparison, to deviate significantly from biological correctness. In addition, each of these unconsidered non-global assay variables can affect the assay RASR values of different particular unregulated gene comparisons, or regulated gene comparisons, to a different significant extent. Individual unconsidered non-global assay variables can affect one unregulated gene comparison in an assay differently, than another unregulated gene comparison in the same assay. A single non-global variable can cause different unregulated genes in the same assay to differ by 1.5 to 10 fold or more, depending on the details of the assay. Because most different regulated gene assay RASRs do not differ by more than 2-10 fold, one unconsidered non-global assay variable can cause two different unregulated gene assay RASR values to be as different or more different than many regulated gene RASR values. In this situation, it would not be possible to distinguish the different unregulated gene assay RASR values from the different regulated gene assay RASR values in the same assay. In plausible assay situations where there are multiple pertinent unconsidered non-global variables associated with an assay, the separate different unconsidered non-global variables associated with one particular unregulated gene comparison, can interact to cause the assay RASR value for that unregulated gene to be different by 1.5 to 40 fold, or more, from a different particular unregulated gene comparison in the same assay. In such a situation many regulated gene assay RASR values in the same assay can be more similar than the particular unregulated genes, making it problematic to distinguish regulated gene RASR values from unregulated gene RASR values. The interaction of the non-global UNFs associated with any particular unregulated gene or regulated gene in the assay, can cause the particular assay RASR value to be smaller, larger, or unchanged, relative to a situation where the unconsidered non-global assay variables are not pertinent to the assay. In such a situation, significant numbers of unregulated gene and regulated gene assay RASR values may have similar or nearly similar RASR values, and because of the RASR value similarity, be erroneously identified as a group of unregulated genes which can be used for normalization purposes. This is most likely to occur in situations where there are large numbers of relatively low mRNA abundance regulated and/or unregulated genes which are active in both compared cell samples, in the assay. Many prior art prokaryotic and eukaryotic, including mammalian, cell comparisons involve such low mRNA abundance gene populations.

Multiple unconsidered non-global assay variables are associated with many if not most prior art microarray and non-microarray gene expression comparison assays. The above discussion indicates that the presence of such unconsidered non-global assay variables makes the identification of the unregulated gene assay RASR values in a particular assay problematic, at best. A consequence of this is that it cannot be assumed that the unregulated genes in an assay can be detected, even when Assumption (i) is valid. Such discussion also indicates that because of the presence of UNFs, distinguishing many unregulated gene assay RASR values from the regulated gene assay RASR values, is also problematic, at best. Furthermore, because of this situation, it cannot be known whether the prior art produced normalization factors for an assay, actually produce biologically correct assay NASR values for each particular gene comparison in a prior art assay. Differences in the linearity of the observed assay signal versus input particular RNA or equivalents. Differences in the accuracy of quantitation of RNA or DNA used in the assay.

The unconsidered non-global assay variables are associated with factors which occur commonly in prior art microarray practice. These include, but are not limited to the following factors. Differences in the nucleotide lengths of the compared LPN molecules. Differences in the total nucleotide complexity (TNC) of the compared LPN molecules. Differences in the CDP molecules present on the array. Differences in the nucleotide sequences of the compared LPN molecules. Differences in the hybridization kinetics of the compared LPN molecules. Differences in the labeling and detection of compared LPN molecules. Differences in the extent of degradation and purity of compared RNAs, and in the isolation efficiencies of total RNA and mRNA.

The existence of multiple prior art unconsidered global and non-global assay variables greatly complicates the interpretation of prior art microarray and non-microarray gene expression comparison assay results. Because prior art microarray practice does not determine or consider these unconsidered assay variables, it is likely that a large fraction of prior art microarray and non-microarray assay NASR values are incompletely normalized, and are biologically incorrect. Since for any particular prior art microarray gene comparison assay, the aggregate effect of the pertinent unconsidered assay variables on each particular gene comparison assay RASR value is not known, the prior art identified unregulated gene assay RASR values, cannot be known to be associated with actual unregulated genes. Consequently, it cannot be known whether Assumption (iii) is valid or not for any particular prior art microarray or non-microarray assay, or whether Assumption (iii) is valid for any prior art gene comparison assay at all.

(iv) The Genes Spotted On the Array Represent A Significantly Large Random Selection of the Total Number of Genes In the Compared Cell Sample.

This assumption is known to be valid for high density microarrays, and prior art acknowledges that Assumption (iv) is not valid for many low density microarrays.

(v) and (vi) The Total RNA Content/Cell is the Same for Each Compared Cell Sample, and/or the Total mRNA Content/Cell is the Same for Each Compared Cell Sample.

As discussed earlier, neither of these assumptions is valid for many prior art microarray and non-microarray gene expression comparison assays. For certain prior art microarray and non-microarray assays it can be known that these assumptions are invalid. For the rest of the prior art microarray and non-microarray gene expression assays, the information is not available to be able to know whether the assumptions are valid or not.

(vii) One or More Genes which are Active in Both Compared Cell Samples are Known to be Unregulated (that is the so Called Housekeeping Genes), and the Assay RASR Results from Such Genes can be Used to Normalize the other Gene Comparisons in the Assay to Produce Biologically Correct Assay NASR Values.

Prior art acknowledges that housekeeping genes with general utility have not been identified. However, a few prior art practitioners believe and practice that unregulated housekeeping genes, which are applicable to particular cell sample comparisons, have been identified. Such limited use housekeeping genes have been identified using prior art microarray and/or non-microarray gene expression analysis methods. As discussed earlier, these prior art microarray and non-microarray gene expression analysis methods do not determine, or take into consideration the prior art unconsidered global and non-global assay variables. Therefore, it cannot be known whether these prior art identified housekeeping genes actually are unregulated, or not. In this context, prior art has never been able to identify a housekeeping gene which can be known to be unregulated in a cell comparison, and thus far there is no evidence that such housekeeping genes exist, even for particular cell sample comparisons.

Prior art has often assumed that certain particular genes in a prior art cell comparison are true housekeeping genes, that is unregulated genes, and used the assay RASR values for these assumed housekeeping genes to normalize the other gene assay RASR values. In such instances, prior art assumed that the particular gene NASRs, which were produced, were biologically correct. Even if it is assumed that such true housekeeping genes actually exist and have been identified, the existence of pertinent non-global assay variables, and in particular the prior art unconsidered non-global assay variables, severely limits the utility of even true housekeeping genes for valid normalization of other particular gene comparison assay RASR values in the same assay. This is discussed below.

Assume that a microarray assay is associated only with pertinent global assay variables, both prior art considered and unconsidered, and is not associated with either considered or unconsidered non-global assay variables. Further, assume that one or more identified true housekeeping genes are present in the cell comparison assay. Here, the assay RASR value for a particular true housekeeping gene is associated with multiple global assay biases, and the aggregate effect of each of these biases can be represented by the product of the NF values for each of the global assay variables. Such product is termed the global variable NF product or GVP. Here, the NF value derived from the housekeeping gene is composed of only the global assay variable GVP value. Consequently, the housekeeping gene derived NF value can be validly used to normalize any other particular gene comparison in the assay to produce biologically correct NASR values. Note that under these conditions where a true housekeeping gene is available, and there are no non-global assay variables associated with the assay, all global assay variables, both considered and unconsidered, and known and unknown, are validly normalized for. Note further that there are no prior art microarray or non-microarray assays where it is known that, identified true housekeeping genes were present in the cell sample comparison, and no non-global assay variables were associated with the prior art assay.

For another assay, assume that only global assay variables and prior art considered non-global assay variables, are associated with the assay. Further, assume that one or more identified true housekeeping genes are present in the cell comparison assay. This situation is much more complex. Here, the housekeeping gene assay RASR value is associated with both global assay variables and non-global assay variables. The aggregate effect of the multiple global assay variables associated with the assay on the housekeeping gene assay RASR value, can be represented by the product of the NF values for each of the assay associated global assay variables NF product, or GVP. This GVP value is associated with every particular gene comparison RASR value in the assay. For this assay, the housekeeping gene assay RASR value is also associated with the aggregate effect of the multiple considered non-global assay variables. The aggregate effect on the housekeeping gene assay RASR of the multiple considered non-global variables, can be represented by the product of the NF values for each of the assay associated considered non-global assay variables which are associated with the housekeeping gene RASR value. The product of the NF values for these considered non-global assay variables associated with the housekeeping gene RASR is the non-global assay variable NF product, or NGVP. This NGVP value is associated with only a subset of particular gene comparisons and is not associated with every particular gene comparison RASR value in the assay. Here, the housekeeping gene assay RASR value is affected by both global and considered non-global assay variables. The aggregate effect on the housekeeping gene assay RASR value of both the global and considered non-global assay variables can be represented by the product of the assay GVP and the assay NGVP. This product represents the NF derived from the unregulated true housekeeping gene assay RASR value. The housekeeping gene NF value is termed the HG-NF value. Thus, the (HG-NF)=(GVP) (NGVP), for a particular housekeeping gene. Multiple true housekeeping genes may be present in the same assay. All of the HG-NF values derived for these housekeeping genes will be associated with the same GVP value. However, because of the assay association with the considered non-global assay variables, each different housekeeping gene HG-NF may be associated with a different NGVP value. Therefore, different HG-NF values would be different, even though all true housekeeping genes are unregulated. In this situation, a particular housekeeping gene's HG-NF value will validly normalize only those other particular gene comparisons, which are associated with the same NGVP value as the particular housekeeping gene. Many prior art microarray and non-microarray gene expression comparisons have assumed the identity of particular housekeeping genes, and used the assay RASR values from these assumed housekeeping genes to normalize all other particular gene comparison RASR values, without taking into consideration any assay associated prior art considered non-global variables. Such a normalization practice is invalid, even if the assumed true housekeeping genes, are true housekeeping genes.

If true housekeeping genes exist, in the above-described situation it should be possible to use existing prior art local normalization methods and herein described methods, in conjunction with endogenous and exogenous replicate controls, to normalize all of the particular gene comparison RASR values, including the housekeeping gene's assay RASR values, for the prior art considered non-global variables which are associated with the HG-NFs. The resulting non-global variable normalized housekeeping gene incompletely normalized NASR value, is then equal to the assay HG-GNFP value which has no NGVP component, and this HG-GNFP value can validly be used to normalize all other particular gene comparisons for the assay global variables. The completely normalized NASR s should then be biologically correct. Note that these normalization approaches will not work unless all of the assay pertinent associated non-global variable biases can be identified and normalized for.

In reality, many, if not virtually all, prior art microarray and non-microarray gene expression comparison assays are associated with multiple considered and unconsidered global assay variables, as well as multiple considered and unconsidered non-global assay variables. As a consequence of the presence of the unconsidered non-global assay variables, prior art assays are even more complex than the above-described hypothetical situation. In reality, a true housekeeping gene derived HG-NF value would represent the product of, (assay GVP)(assay considered NGVP)(assay unconsidered NGVP). Prior art normalization practice does not take unconsidered non-global assay variables into account when determining the prior art version of the HG-NF.

The above discussion on the validity of Assumption (vii) indicates the following. (a) Prior art generally acknowledges that general use housekeeping genes have not been found. (b) Prior art identified and used putative housekeeping genes were identified using gene expression analysis methods which did not take unconsidered assay variables into consideration, and therefore such genes cannot be known to be true housekeeping genes. (c) Even if true housekeeping genes did exist, their prior art use for valid normalization of other particular gene comparison results is not valid due to the association of prior art microarray and non-microarray assays with unconsidered assay variables. In this context Assumption (vii) is invalid for prior art microarray and non-microarray gene expression comparison assays.

Validity of Prior Art Normalization Assumptions: Summary.

The conclusions regarding the validity of the prior art assumptions which are required for one or another prior art normalization approach, are presented below.

| | |
|---|---|
| Assumptions (i) & (ii) | Assumptions are not valid for certain prior art microarray and non- |
| | microarray and assays, and may not be valid for many of these |
| | assays. Further, with few exceptions, it is not possible to know |
| | whether the assumption is valid for any particular prior art assay. |
| Assumption (iii) | While it is likely that the assumption is invalid for many prior art |
| | microarray and non-microarray assays, it cannot be known for any |
| | particular assay whether the assumption is invalid or not. The |
| | assumption may be invalid for all prior art assays. |
| Assumption (iv) | Assumptions are known to be valid for high density microarray |
| | assays, and is not valid for many low density microarray assays. |
| Assumptions (v) & (vi) | Assumptions are known to be invalid for certain prior art |
| | microarray and non-microarray assays, and is likely to be invalid |
| | for many other prior art assays. |
| Assumption (vii) | Assumption is invalid. |

Of the seven required prior art normalization assumptions, six are either invalid or have questionable and unknown validity for prior art assays.

Even if all of these prior art assumptions are valid for an assay, it cannot be assumed that the prior art normalization process produces validly normalized particular gene comparison NASR values which are accurately normalized for all pertinent global and non-global CNFs and UNFs. Such prior art normalization processes include the various global prior art normalization approaches and prior art local normalization approaches. Such approaches include those using the global and local intensity approaches of various kinds and those, which include spike in controls.

H. Validity of Prior Art Interpretation of Microarray and Non-Microarray Assay Measured Particular Gene Expression Results

Occurrence of EA Rule Related False Negative Gene Activity Results and Regulation Direction Miscalls Associated with (ACR)≠(T-DGER).

A very important aspect of gene expression analysis is the identification of active genes in a cell sample. Also very important is the determination of whether the same gene is active in different cell populations. This is usually accomplished by the direct comparison of the total RNA, total mRNA, or equivalents, from two or more cell populations. Many of these direct comparisons indicate that a particular gene is active in one cell sample, but not in another cell sample. The standard interpretation of this situation is that the number of mRNA copies per cell for the particular gene in the “active” cell sample, is higher than the number of mRNA copies per cell for the same gene in the “inactive” cell sample. As a consequence, the gene in the “inactive” cell sample is regarded as being downregulated, relative to the same gene in the “active” cell sample. For many EA Rule related gene activity comparisons, this interpretation cannot be known to be correct. The reasons for this are discussed below.

As discussed earlier, a consequence of the practice of the EA Rule for microarray or non-microarray gene expression analyzes which compare cell samples which have different total RNA or total mRNA contents per cell, is that unequal numbers of each sample's cells are often compared in the assay. This then, creates an assay situation where the relative amounts of a particular gene's cell sample mRNA transcripts which are present in the assay hybridization solution, does not accurately reflect the relative amounts of the gene's particular mRNA transcripts which are present in the average cell of each compared cell sample. Thus, relative to the actual situation present in the average cell of each compared cell sample, the amount of LCN sample particular mRNA transcript present in the comparison assay hybridization solution is underrepresented. Therefore, in this situation the ACR for the particular mRNA transcript in the assay hybridization solution is not equal to the T-DGER for the particular mRNA transcript, which exists in the compared cell samples. Put differently, for the particular gene comparison, the (ACR)≠(T-DGER). When a microarray or non-microarray assay particular gene comparison, is associated with a situation where the (ACR)≠(T-DGER), an EA Rule or SCR related false negative result and RDM can occur. The occurrence of such false negative results is discussed below. Since prior art microarray and non-microarray gene expression analysis assays almost always involve an SGDS comparison of particular gene mRNA transcripts, the discussion will primarily concern these prior art assays. However, the discussion applies directly to all SGDS, DGDS, and DGSS comparisons of viral, prokaryotic, and eukaryotic RNAs of all kinds. This includes all types of rRNA, tRNA, mRNA, siRNA, miRNA, snoRNA, antisense RNA, and other known and unknown RNAs.

The nature of gene activity comparison assay true positive and false negative results were discussed earlier. In the context of an EA Rule related gene activity comparison assay, in which the HCN sample gives a positive result for a particular gene, while the LCN sample gives a negative result of the same gene, there are two different types of LCN sample false negative results. The first, termed an EA Rule false negative, results from the EA Rule practice related under-representation of the LCN sample mRNAs in the assay. This EA Rule false negative result can be converted to a true positive, by increasing the number of LCN sample cells in the assay so that equal numbers of sample cells are compared in the assay. In addition, the EA Rule false negative result causes a gene regulation direction miscall. The second type is termed a non-EA false negative. The non-EA false negative results in a correct gene regulation direction call, and indicates that an LCN sample gene which gives a false negative result is downregulated, relative to the same gene in the HCN sample which gives a true positive result. Only the EA Rule false negative results will be discussed below.

To simplify the analysis of this problem, the discussion will be presented in terms of a standard microarray comparative gene expression analysis, which compares the total RNA of two cell samples. However, the discussion will be directly applicable to the use of total RNA equivalents, or total mRNA or equivalents, as well as to other non-microarray methods of comparative gene expression analysis, including northern blotting, dot blotting, nuclease protection, and RT-PCR. The discussion will assume that, the EA Rule is practiced in an ideal way, and that equal amounts of total RNA from each cell sample are added to the microarray hybridization solution and that the prior art belief that (N-DGER)=(NASR)=(ACR), is true. This practice means that the ratio of the amounts of each sample's total RNA being compared is equal to one for every separate microarray gene expression comparison analysis. This discussion concerns the effect of always using the same ratio of input sample total RNA in a gene expression analysis, on the interpretation of the results. Note that while only one method of fixing this ratio, the EA Rule, is discussed, the discussion applies directly to any other non-EA Rule method of fixing the ratio of sample amounts added to all microarray hybridization solutions.

Earlier sections have established five key points. First, the amount of each samples total RNA, total mRNA, or equivalents, added to the gene expression assay determines whether a detectable quantity of a particular gene's mRNA transcript is present in the assay. Further, changing the amount added can change the assay gene activity measurement result from positive to negative, or from negative to positive. Second, the amount of sample RNA available for a comparative gene activity assay is, very often, not enough to ensure that all, or even a majority of the low abundance mRNA transcripts will be detected. This is especially true for mammalian comparisons. Third, it is common for a large number of the same genes to be transcriptionally active in each sample being compared. This is especially true in mammals, where thousands of the same genes produce low abundance mRNAs in different cell samples. Fourth, significant differences in the total RNA content per cell, and total mRNA content per cell, are common for different cell samples. Fifth, virtually all gene activity comparisons practice one form or another of the EA Rule to determine the ratio of each sample's RNA, which is present in a comparison assay. A consequence of this is that unequal numbers of cells from each sample are usually compared because different cell samples have different RNA contents per cell.

It will be useful for this analysis to discuss the just detectable amount of an mRNA in a microarray assay. This discussion will utilize generally accepted parameters from the literature and other sources. These parameters will be used to relate the just detectable amount or quantity of a mRNA in a standard microarray assay, to the amount of total RNA, or total mRNA added to a microarray hybridization solution, and to the abundance level of the mRNA which is just detectable with a particular amount of a samples input total RNA, or total mRNA. Herein, the just detectable quantity of a particular cell sample mRNA, or nucleic acids derived therefrom, in the assay hybridization solution is termed the JDQ.

The JDQ is determined by a variety of factors, including the hybridization solution composition, volume, and temperature, as well as the hybridization reaction time. For a given microarray assay system, these factors are fixed. The JDQ of a particular gene's RNA LPN in an assay, is also affected by the characteristics of the particular gene LPN in the assay hybridization solution, as well as the characteristics of the Complementary Detection Polynucleotide (CDP) utilized for the assay to detect the particular gene LPN. For a microarray particular gene comparison of the expression of the same gene in different cell samples, the JDQ of each cell sample's particular gene mRNA LPN molecules is the same, when the assay hybridization conditions and the compared LPN molecule characteristics are the same for each compared cell sample's particular gene mRNA LPN molecules. For such a gene comparison, the ratio in the assay hybridization solution of, (the JDQ for the particular gene mRNA LPN molecules associated with one cell sample)÷(the JDQ for the same particular gene mRNA LPN molecules associated with a different cell sample), is equal to one. Herein, this assay JDQ ratio is termed the assay JDQR.

The JDQ for a cell sample particular gene mRNA LPN in a gene comparison assay, represents the minimum amount of particular mRNA LPN which can be detected in the gene comparison assay system, and as such, the JDQ is independent of the amount of the particular gene mRNA LPN which is actually present in the assay itself. Thus, for a given microarray assay system, the JDQ of a particular gene mRNA LPN with particular LPN characteristics, is fixed, and is not influenced by the amount of a particular mRNA LPN in the assay. Therefore, the assay JDQR value is also not influenced by the assay SCR, PAFR, or ARR values. In other words, the practice or non-practice of the EA Rule for a gene comparison assay has no influence on the assay JDQR for the gene comparison. For the purposes of this discussion on the occurrence and interpretation of EA Rule false negative results, it will be assumed that the assay JDQR=1, for all illustrations.

The occurrence of microarray and non-microarray EA Rule related false negative results, can be prevented by adding enough RNA or RNA LPN from each cell sample to the assay hybridization solution, to ensure that every high or low abundance mRNA present in the compared cell samples, is present in the assay hybridization solution in an amount equal to or greater than the JDQ for each mRNA LPN. In reality, this is rarely possible, as discussed below.

An average mammalian cell has a total RNA/DNA ratio of about two, contains a total of about 300,000 mRNA transcript molecules, and has about 0.02 of its total RNA as total mRNA (1, 5, 7, 26, 27). A particular mRNA type present at one copy per cell would then be present at a frequency of 1 in 300,000. It is usually assumed that an average mammalian mRNA molecule contains about 1,800 bases, has a molecular weight of about 6×10⁵ Daltons, and a mass of about 10⁻¹⁸ grams. It should be noted that the above quoted values are averages, and that for any specific real life situation the average value could significantly differ from reality. As an example, the total RNA/DNA ratio for different mammalian cell samples can range from about 1/5 to 5/1; the number of total mRNA transcripts per mammalian cell can range from about 10⁵ to 10⁶; and the fraction of total RNA consisting of mRNA can range from 0.01 to 0.05. For simplification of the discussion on the just detectable amount of an mRNA transcript, the average values will be used.

A typical gene expression analysis glass microarray assay employs a hybridization solution volume of around 20 microliters, and a hybridization incubation time of 10-15 hours. For this condition, the just detectable amount of an mRNA transcript is about 10⁷ mRNA transcript molecules or equivalents (5). This results in a just detectable mRNA transcript concentration of 8×10⁻¹³ M. By definition, the number of cells which contain a total of 10⁷ single copy per cell mRNA transcripts, is 10⁷ cells. In this system, the minimum amount of purified total mRNA which contains a just detectable amount of a one copy per cell mRNA transcript is equal to, (the number of sample cells required for a just detectable amount)×(number of total mRNA molecules per cell), or 3×10¹² total mRNA molecules. This is equivalent to about three micrograms of purified total mRNA, since each mRNA weighs about 10⁻¹⁸ grams. It is often assumed that the fraction of total mammalian RNA which consists of total mRNA transcripts is 0.02. Assuming this, the amount of total cell RNA which contains three micrograms of total mRNA is about 150 micrograms of total mammalian cell RNA. Thus, in order to just detect one copy per cell mRNA in an average mammalian cell, the amount of total cell RNA which must be added to the 20-microliter-volume hybridization solution is 150 micrograms. Alternatively, the amount of purified total mRNA, which must be added, is three micrograms. These are the amounts of total RNA and total mRNA present in 10⁷ average mammalian cells. In reality, the amount of mammalian sample total RNA, or total mRNA, added for gene activity comparisons is often much less. In the above context, when the gene comparison assay mRNA LPNs have the same nucleotide length, the same nucleotide sequence, the same nucleotide complexity, the same Total Polynucleotide Number (TPN), and the same appropriately high label signal activity per mass of LPN, the JDQ for each particular compared mRNA LPN in the assay is the same, or about 8×10⁻¹³ M, and the assay JDQR equals one.

This illustration can be extended to examine the effect of the amount of total RNA or total mRNA present in the microarray hybridization solution on the abundance level of the cell mRNA transcripts which are present in the just detectable mRNA fraction. This is illustrated in Table 32B. As the number of sample cells decreases, the abundance level of the just detectable cell mRNA fraction increases proportionally. It is common to utilize 0.5 to 1 microgram of purified mRNA, or its total RNA equivalent, to produce labeled cDNA, which is then added to the microarray hybridization solution. It is not uncommon to utilize more mRNA, or less. In this example based on an average mammalian cell, at one microgram added total mRNA, just detectable mRNAs have an abundance of about three mRNA transcripts per cell. In reality, at this total mRNA input, the just detectable mRNA abundance level can range from less than one mRNA copy per cell, to about nine copies per cell or more, depending on the mammalian sample types being compared. In real life mammalian microarray gene activity comparisons, the just detectable mRNA's abundance class is rarely as low as one mRNA copy per cell, even for the comparison of homogeneous populations of cells. Herein the just detectable abundance level for a particular gene RNA in a cell is termed the JDA.

TABLE 32B
Sample Cell Number Versus Just Detectable Abundance Level in
Microarray Gene Expression Assay

| ^{(a)}Just Detectable | | Micrograms of Input | | Just Detected |
|---|---|---|---|---|
| Amount of a | Number | RNA in Hybridization | | Abundance |
| Particular | of Cells | Solution | | Level in |
| mRNA | in Sample | Total^{(c)} | mRNA | Cell^{(b)} |
| 10⁷ Molecules | 10⁹ | 15,000 | 300 | 0.01 |
| 10⁷ Molecules | 10⁸ | 1,500 | 30 | 0.1 |
| 10⁷ Molecules | 10⁷ | 150 | 3 | 1 |
| 10⁷ Molecules | 10⁶ | 15 | 0.3 | 10 |
| 10⁷ Molecules | 10⁵ | 1.5 | 0.03 | 100 |
| 10⁷ Molecules | 10⁴ | 0.15 | 0.003 | 1,000 |
| 10⁷ Molecules | 10³ | 0.015 | 0.0003 | 10,000 |
| 10⁷ Molecules | 10² | 0.0015 | 0.00003 | 100,000 |

^{(a)}Hybridization solution volume equals 20 microliters placed on a glass slide. Average mRNA length equals 1,800 bases 0.02 of total RNA as mRNA Incubation time 10-15 hours
^{(b)}Abundance level represents the number of copies per cell for a particular mRNA.
^{(c)}Assumes a total RNA to DNA ratio of 2/1, and a diploid cell DNA content of 7.5 picograms per mammalian cell.

As described above, the just detectable amount of an mRNA in a typical glass microarray assay system is 10⁷ mRNA transcript molecules. Also, the amount of average mammalian cell total RNA which must be added to the hybridization solution in order to just detect one copy per cell mRNA transcripts, is 150 micrograms. The average mammalian cell is generally assumed to have a total RNA/DNA ratio of about two, while in reality the total RNA/DNA ratio of different mammalian cell samples ranges from 0.2 to around 5. In this context, it will be useful to determine the effect of the mammalian cell sample total RNA/DNA ratio on the amount of total RNA from a particular mammalian cell sample which is required in order to attain a just detectable abundance level of one mRNA transcript per cell in a microarray gene activity comparison assay. The results are presented in Table 33. These results show that the amount of total RNA required to detect the one copy per cell abundance level goes up or down proportionally with the total RNA/DNA ratio of the mammalian cell type assayed, and can differ by twenty-five fold, depending on the cell sample.

TABLE 33
Effect of Sample Total RNA/DNA Ratio on Amount of Total RNA Necessary
to Detect One mRNA Transcript Copy/Cell

| | | | Number of Cells | |
|---|---|---|---|---|
| | | Just Detectable | Necessary to | |
| | | Number of | Yield 10⁷ mRNA | |
| | Total RNA | mRNA | Copies of One | ^{(b)}Required |
| Mammalian | DNA | Transcripts in | Copy mRNA Per | Amount of Total |
| Cell Sample | Ratio^{(a)} | Assay | Cell | RNA |
| Average | 2/1 | 10⁷ | 10⁷ | ˜150 micrograms |
| Mammalian Cell | | | | |
| Rat Adult | 0.17/1 | 10⁷ | 10⁷ | ˜13 micrograms |
| Thymus Cell | | | | |
| Rat Adult Liver | 4.3/1 | 10⁷ | 10⁷ | ˜323 micrograms |
| Cells | | | | |

^{(a)}See Table 1.
^{(b)}Amount of total RNA added to 20-microliter hybridization solution, which will give a just detectable abundance level of one mRNA copy per cell.

The following discussion is designed to analyze the effect of one factor, the practice of the EA Rule, on the interpretability of a negative or inactive result for a particular gene in one sample, when the same gene is detected as being active in another cell sample being compared. It is important to emphasize that for this analysis, the existence of any identified interpretation problem is independent of the workings of the microarray assay itself, and that the problem is caused by the EA Rule dictated composition of the microarray hybridization solution. Thus, the problems are intrinsic to the use of the EA Rule. In this context, the discussion has assumed that the microarray assay itself works perfectly, and that the EA Rule is practiced ideally. It has also been assumed that the process of obtaining cell samples and isolating and quantitating total RNA, and total mRNA, works perfectly, and that all total RNA or total mRNA equivalents perfectly reflect the qualitative and quantitative characteristics of the natural RNA populations used to produce them, and that the only significant assay variable is the use of the EA Rule. Any imperfections in these assumptions would increase the magnitude of the interpretability problem already existing due to the practice of the EA Rule.

In this context, microarray results concerning the active or inactive status of a particular gene in a sample, reflects the amount of the gene's mRNA transcripts which is present in the microarray hybridization solution. If a detectable amount of the gene's mRNA transcripts is present in the amount of the samples total RNA which has been added to the hybridization solution, then the gene is reported to be active. In the microarray practice of the EA Rule for gene activity detection, the measurement units are in terms of the amount of a gene's mRNA transcripts per hybridization solution, and the amount is either detectable or undetectable. In a comparative gene expression analysis, which practices the EA Rule, these measurement units are adequate for unambiguously establishing the presence of an actively expressed gene. These units are also adequate for the unambiguous intercomparison of active genes identified in different microarray or non-microarray gene comparison analyzes, involving different samples and different conditions. In simple words, with these EA Rule dictated measurement units, a positive result is readily interpretable. A positive result means that the gene is active in the sample. A positive result for the gene for both samples, means that the gene is active in both samples.

While the microarray practice of the EA Rule does not cause any problems in interpreting whether a result is positive or not, it can lead to erroneous conclusions about negative results when, in a gene comparison, a particular gene is measured to be active in one sample, and inactive in the other sample. Large numbers of such results are obtained in microarray comparisons of mammalian cell samples, and the great majority of these results occur for the low abundance mRNA. As discussed earlier, in a typical mammalian cell somewhat more than 12,000 different genes are expressed as mRNA. The mRNA transcripts from about 10,000 different genes constitute the low abundance mRNA fraction in a typical mammalian cell. In different mammalian cell samples, thousands of the same genes are active and produce low abundance mRNA. In each of these different mammalian cell samples, there are also thousands of different genes which produce low abundance mRNA and which are active in one cell sample and not another. Currently, the accepted interpretation of this situation is that gene's extent of expression is higher in the sample where it is measured to be active, than in the sample where the gene is measured to be inactive. In other words, the prior art accepted interpretation indicates that the number of the gene's mRNA transcripts per cell in the “active” sample, is greater than the number of the same gene's mRNA transcripts per cell in the “inactive” sample. As a result, the gene in the “active” sample would be regarded as being upregulated, relative to the gene activity of the “inactive” sample. Because of the practice of the EA Rule, and the existence of significant natural differences in the total RNA content per cell and total mRNA content per cell in different cell samples, this interpretation cannot be known to be true. It is possible that the microarray negative result for gene expression activity in one sample is a false negative and that, in reality, the gene may be expressed to an equal or greater extent per cell in the inactive sample than in the active sample, but its expression is not detectable because of the practice of the EA Rule. This situation can occur because the microarray practice of the EA Rule dictates that the gene activity results are measured in terms of a detectable or undetectable amount of a particular gene's mRNA transcripts per microarray hybridization solution, and no effort is made to relate these measurement units to the number of each samples cell equivalents which are present in a microarray hybridization solution. The number of sample cell equivalents for one cell sample is the number of sample cells, which contain the amount of total RNA, total mRNA, or equivalents, which is present in the hybridization solution. The ratio in a microarray hybridization solution of, (the number of one sample's cells, which are present)÷(the number of the other sample's cells, which are present), is termed the hybridization solution cell ratio, or SCR. The ratio of the number of each sample's cells, which are directly compared in a gene expression comparison assay, is also termed the sample cell ratio, or SCR.

An EA Rule related false negative result for a particular gene is certain to occur in gene activity sample comparisons which meet all of the following criteria. First, the EA Rule must be practiced for the comparison. Second, the total RNA content per cell or total mRNA content per cell must be different for each sample compared. This, along with the practice of the EA Rule will result in unequal sample cell numbers being compared, and the Sample Cell Ratio (SCR) for the comparison assay will not be equal to one. For simplification, the sample which contributes the most cells to the comparison is designated the High Cell Number (HCN) sample, while the other sample is designated the Low Cell Number (LCN) sample. The LCN sample has a larger total RNA content per cell or total mRNA content per cell, than the HCN sample. Third, a particular gene must be actively expressed in each sample being compared. Fourth, the particular gene's cell mRNA abundance level in the HCN sample, must be equal to or less than, the same gene's LCN sample mRNA abundance level. Therefore, the particular gene's LCN sample mRNA abundance level, must be equal to or greater than, the same gene's HCN sample mRNA abundance level. Fifth, a detectable amount of the gene mRNA LPN from the HCN sample must be present in the assay hybridization solution. Put differently, the particular genes HCN sample mRNA abundance level must be detectable in the assay. Sixth, an undetectable amount of the gene mRNA LPN from the LCN sample must be present in the assay hybridization solution. Put differently, for the particular gene comparison, the magnitude of the deviation of the assay SCR value from one, must be great enough so that the gene's mRNA abundance in the LCN sample, is not detectable in the assay, even though the gene's LCN sample mRNA abundance level is equal to or greater than, the gene's HCN sample mRNA abundance level in the same assay.

The occurrence of EA Rule related false negative results, can be illustrated with the mouse fibroblast 3T3 growing and non-growing cell samples described earlier. The total RNA content per growing 3T3 cell, is four times larger than that of non-growing 3T3 cells, and the total mRNA content per growing cell is six times that of non-growing 3T3 cells. For the purpose of this illustration, the following will be assumed. (a) Equal amounts of LCN growing and HCN non-growing 3T3 cell total RNA's are present in the microarray hybridization solution. This results in an SCR of 0.25, and more non-growing cells than growing cells in the hybridization solution. (b) The mRNA of a particular gene is present at one copy per cell in both LCN growing and HCN non-growing cells. (c) The amount in the microarray hybridization solution of the particular gene's HCN non-growing cell mRNA transcripts in the microarray system, and the just detectable HCN non-growing cell abundance level is one mRNA copy per cell. (d) The JDQR is equal to one.

As a consequence of the practice of the EA Rule, which results in comparing unequal numbers of sample cells, the microarray hybridization solution contains a detectable amount of the HCN non-growing cell particular gene mRNA transcripts, and an undetectable amount of LCN growing cell mRNA transcripts from the same gene. This microarray assay will yield a positive result for the HCN non-growing cell particular gene, and a negative result for the same gene in the LCN growing cells. The standard interpretation of these results would be that the particular gene is not active in the LCN growing cells, and that the gene was downregulated in growing cells, relative to HCN non-growing cells. This result is an EA Rule related false negative result because, in reality, the particular gene is expressed to the same extent per cell in both non-growing and growing cells. In addition, in reality there is no change in regulation direction between growing and non-growing cells. This illustration is summarized in Table 34. This table also illustrates the occurrence of EA Rule related false negatives in a comparison of total mRNA from growing and non-growing 3T3 cells, by assuming different numbers of mRNA copies per cell for the two sample. In the mRNA comparison, the microarray result was negative for the LCN growing cells even when the LCN growing cells contained five mRNA copies per cell, and the HCN non-growing cells had only one copy per cell. A similar result was observed for the total RNA comparison. The SCR's of the total RNA and total mRNA comparisons were 0.25 and 0.166 respectively. For the mRNA comparison, the mRNA abundance range over which the false negatives occurred in the LCN sample, was from one mRNA copy per cell to almost six mRNA copies per cell, while the comparable range for the total RNA comparison was from one to almost four mRNA copies per cell. Clearly, it cannot be assumed that the total mRNA and total RNA from the same cells will give the same pattern of false negative results. This also indicates that the farther the SCR deviates from one, the greater the mRNA abundance range in the LCN growing cell sample, over which EA Rule false negatives can occur.

TABLE 34
EA Rule Related False Negative Gene Activity Results: Growing and Non-
Growing 3T3 Cell Comparison

| | | Assumed | | | | | | | |
|---|---|---|---|---|---|---|---|---|---|
| | | Number | | Relative | | | | | |
| | | of mRNA | | Amount | | Microarray | | EA | |
| | | Copies | | of Gene's mRNA | | Gene | | Rule Related | |
| 3T3 Cell | | Per Cell | | in Hybridization | | Activity | | False Negative | |
| RNA | (G/NG) | for Gene | | Mix | | Result | | Result | |
| Compared | SCR^{(a)} | G^{(d)} | NG | G | NG^{(b)} | G | NG | G | NG |
| Total RNA | 0.25 | 1 | 0.99 | 0.25 | 0.99 | NEG | NEG | | |
| | 0.25 | 1 | 1 | 0.5 | 1 | NEG | POS | YES^{(c)} | |
| | 0.25 | 2 | 1 | 0.75 | 1 | NEG | POS | YES^{(c)} | |
| | 0.25 | 3 | 1 | 1 | 1 | NEG | POS | YES^{(c)} | |
| | 0.25 | 4 | 1 | 1 | 1 | POS | POS | | |
| mRNA | | 1 | 0.99 | 0.166 | 0.99 | NEG | NEG | | |
| | 0.166 | 1 | 1 | 0.166 | 1 | NEG | POS | YES^{(c)} | |
| | 0.166 | 2 | 1 | 0.333 | 1 | NEG | POS | YES^{(c)} | |
| | 0.166 | 4 | 1 | 0.666 | 1 | NEG | POS | YES^{(c)} | |
| | 0.166 | 5 | 1 | 0.833 | 1 | NEG | POS | YES^{(c)} | |
| | 0.166 | 6 | 1 | 1 | 1 | POS | POS | | |

^{(a)}EA Rule is practiced.
^{(b)}A value of one for the NG mRNA transcripts represents a just detectable amount in this microarray analysis system and the just detectable NG sample abundance level is one mRNA copy per cell.
^{(c)}Regulation direction change indicated is also false.
^{(d)}G - growing sample is the LCN sample
NG - non-growing sample is the HCN sample

This can be further illustrated by a comparison of the total RNA's from adult rat liver and thymus samples. In the practice of the EA Rule the SCR=0.04 for this comparison when the thymus cells are in the denominator. This indicates that the liver total RNA content per cell is 25 times greater than that of the thymus cells (see Table 1). For this illustration, the following will be assumed. (a) The SCR=0.04. (b) The mRNA of a particular gene is present at one copy per thymus cell, and at varying mRNA copy per cell numbers for liver cells. (c) The amount in the microarray hybridization solution of the particular gene's HCN thymus cell mRNA transcripts, equals the just detectable amount of mRNA transcripts in the microarray assay system, and the just detectable HCN thymus cell abundance level is one copy per cell. Table 35 presents the results of this example. At an SCR of 0.04, the mRNA abundance range in the LCN liver sample over which false negatives can result extend from one mRNA copy per cell to about 25 mRNA copies per cell. The results of Tables 34 and 35 indicate this range increases in direct proportion to the extent of deviation of the SCR from one, and decreases as the SCR approaches one. Clearly at (SCR=1), no EA Rule related false negatives will occur.

TABLE 35
EA Rule Related False Negative Gene Activity Results: Comparison of Rat
Liver and Thymus Samples

| | Assumed | | | | | | |
|---|---|---|---|---|---|---|---|
| | Number | | | | | | |
| | of mRNA | | | | | | |
| | Copies | | Relative Amount of | | Microarray Gene | | EA Rule Related |
| | Per Cell | | Gene's mRNA in | | Hybridization | | False Negative |
| (Liver/Thymus) | for Gene | | Mix | | Activity Result | | Result |
| SCR^{(a)} | Liver | Thymus | Liver | Thymus^{(b)} | Liver | Thymus | Liver^{(d)} |
| 0.04 | 1 | 1 | 0.04 | 1 | NEG | POS | YES^{(c)} |
| 0.04 | 10 | 1 | 0.4 | 1 | NEG | POS | YES^{(c)} |
| 0.04 | 20 | 1 | 0.8 | 1 | NEG | POS | YES^{(c)} |
| 0.04 | 24 | 1 | 0.96 | 1 | NEG | POS | YES^{(c)} |
| 0.04 | 25 | 1 | 1 | 1 | POS | POS | |

^{(a)}EA Rule is practiced.
^{(b)}The amount of thymus mRNA present is a just detectable amount in this microarray system, and the HCN thymus just detectable abundance level is one mRNA copy per cell.
^{(c)}Also falsely indicates that the gene is downregulated in growing cells.
^{(d)}Liver is LCN sample, and thymus is HCN sample.

Do EA Rule and (ACR≠T-DGER) Related False Negatives Occur in Real Life?

The above discussions establish that EA Rule related false negative gene activity results will occur under certain conditions, and cannot occur under other conditions. The obvious question concerning the relevance of this to real life prior art gene activity measurements arises, and will be discussed below. This discussion will be presented in terms of the same microarray gene activity comparison used in the above analysis. The discussion will be directly applicable to other non-microarray gene activity measurement methods.

EA Rule related false negative results are certain to occur in real life gene activity comparisons if all 6 of the earlier described criteria are met for one or more genes. This discussion will investigate the extent to which each criterion is known to be met in standard microarray and non-microarray gene activity comparison practice.

The first requirement specifies that the EA Rule must be practiced for the particular gene comparison. In this event, the EA Rule related false negative results can occur only when unequal numbers of sample cells are compared. For a specific mRNA transcript present in each compared sample, this creates a situation where the relative amounts of each sample's mRNA transcripts which are present in the comparative assay, do not reflect the relative amounts of the specific mRNA transcripts which are present in the average cell of each sample. Thus, relative to the actual situation present in the average cell of each compared sample, the amount of the LCN sample specific mRNA present in the comparison assay is under-represented. A consequence of this is that the LCN sample specific mRNA can be present at an undetectable amount in the gene activity comparison, even though the LCN sample specific mRNA per cell number is equal to or higher than that for the HCN sample. As discussed earlier, this requirement is certainly met.

The second requirement specifies that the total RNA content per cell, or total mRNA content per cell, must be different for each sample compared. This, along with the practice of the EA Rule, will result in unequal sample cell numbers being compared, and the sample cell ratio, or SCR, for the comparison assay will not equal one. In this situation, one sample is the High Cell Number (HCN) sample, and the other is the Low Cell Number (LCN) sample. In the practice of the EA Rule, this condition is not met only when the total RNA contents per cell, or total mRNA contents, of the compared samples are equal, or in other words, when the sample cell ratio is equal to one. The available information indicates that the total RNA content, per cell, or the total mRNA content per cell, is often not the same in different cell samples. Indeed, as discussed earlier for bacteria and mouse fibroblast 3T3 cells, the total RNA, or total mRNA, contents per cell of a single homogeneous population of cells can vary by four to ten fold, depending on the growth stage of the cells. Thus, in a comparison of the same cells, the EA Rule dictated SCR can vary by 1 to 6 fold in mammalian 3T3 cells, and 1 to 10 fold in bacteria cells, depending on the growth stage of the cells. The total RNA per cell contents of different mammalian cells from the same organism can vary by twenty-five fold. Thus, different types of mammalian cell samples seldom have the same total RNA content (see Table 1). This indicates that in for many prior art microarray, and non-microarray gene activity comparison assays, the SCR is not equal to one. Further, it is likely that many of these prior art assays have SCR values which deviate from one by a factor of two or more, whether T-RNA or isolated mRNA is compared.

The third required condition for the certain occurrence of an EA Rule related false negative result for a particular gene comparison, specifies that the particular gene must be actively expressed in each compared cell sample. As discussed earlier, in real life prior art gene comparisons this condition is almost always met for thousands of genes in each compared cell sample. This is particularly true for mammalian cell sample gene activity comparisons where over 10,000 different genes are reported to be actively expressed in a typical mammalian cell sample comparison, and well over half of these different genes are expressed in both compared cell samples as low mRNA abundance mRNA transcripts. In addition, the abundance of the commonly expressed low abundance mRNA transcripts, is similar but not necessarily identical, in each different cell sample. This large overlap between the low abundance mRNA populations of different related mammalian and other cell types, is common for mammalian, and other eukaryote and prokaryote cell types, and their neoplastic offshoots. All this indicates that in real life prior art microarray mammalian gene activity comparisons, the third requirement is met for the mRNA transcripts of as many as 5,000 different active genes.

The Fourth requirement specifies that the particular gene's cell mRNA abundance level in the HCN sample, must be equal to or less than, the same gene's LCN sample mRNA abundance level. As discussed earlier, each cell sample in a mammalian cell sample gene comparison contains 12,000-15,000 active genes, and about 10,000 or so of these active genes are low mRNA abundance level genes which have an abundance level of 1-5 mRNA copies per cell. Over half the 10,000 or so low abundance mRNA genes are active in both compared mammalian cell samples, while the rest are detected as being active in only one cell sample. For simplicity it will here be assumed that for a mammalian cell comparison, about 5,000 low abundance 1-5 mRNA copy per cell genes are detected as being active in both compared cell samples, and about 5,000 low abundance 1-5 mRNA copy per cell genes are detected as being inactive in one cell sample and active in the other. Thus, for a mammalian cell sample gene comparison: 5,000 or so different low abundance 1-5 mRNA copy per cell genes are active in both cell samples, and an active gene in one cell sample has a mRNA abundance level which is equal to or similar to the abundance level of the same gene in the compared cell sample; 5,000 or so different low abundance 1-5 mRNA copy per cell genes are detected as active in one cell sample and not the other, and each detected active gene in one cell sample has a mRNA abundance level which is similar to the mRNA abundance level of the same gene in the other compared cell sample. Prior art generally believes that for those genes which are active in both cell samples, and differentially expressed, about half are downregulated in one cell sample, and upregulated in the other cell sample. Thus, for a particular differentially expressed gene in a gene comparison assay, the probability of the downregulated gene being associated with the LCN sample is about 0.5. In addition, the probability of the upregulated gene being associated with the HCN sample, is about 0.5. Therefore, roughly one quarter of differentially expressed particular prior art genes meet this fourth requirement for both the LCN and HCN samples.

Prior art also commonly practices that for a typical cell sample gene comparison assay, the great majority of those genes which are active in both cell samples, are unregulated. Unregulated indicates that, a particular active gene has the same gene mRNA abundance level in each compared cell sample. In this event, for eukaryotic and prokaryotic cell sample gene comparisons, the majority of active in both cell samples genes, are unregulated low mRNA abundance genes. For mammalian cell sample gene comparisons, as many as 4,000-5,000 active in both cell samples genes, are unregulated, low mRNA abundance genes. Therefore, for many prior art eukaryotic and prokaryotic gene comparisons, the particular active gene's mRNA abundance in the LCN sample, is equal, or nearly equal to the same gene's mRNA abundance in the HCN sample, and the fourth requirement is met. For prior art mammalian gene comparisons, this fourth requirement is met for 4,000-5,000 different particular low cell mRNA abundance genes.

A typical prior art microarray cell sample comparison detects as active a large number of low mRNA abundance level genes in each cell sample, and does not detect as active the same genes in the other cell sample. For a high density mammalian microarray, hundreds to thousands of low mRNA abundance level genes may be detected as being active in only one cell sample of the comparison. While the nature of these active in one cell sample low mRNA abundance genes is not known, many of them could meet this fourth requirement.

The fifth requirement specifies that a detectable amount of a particular gene mRNA LPN from the HCN sample, must be present in the assay hybridization solution. Put differently, the particular gene's mRNA abundance level in the HCN sample, must be detectable in the assay. As discussed above, for a typical mammalian cell sample about 10,000 genes are associated with the low abundance mRNA class of about 1-5 copies per cell. For a typical mammalian cell sample gene expression comparison, about 6,000 or so genes which are associated with low abundance mRNAs are believed to be active in both compared cell samples, and most of these are unregulated genes or nearly unregulated genes. Further, many prior art microarray assays are associated with a JDAs which allows the detection for the HCN cell sample of an mRNA abundance level of about 3 CPC. For a typical mammalian cell sample comparison it appears likely that a thousand or so unregulated low abundance mRNA genes will be associated with the 3 copy per cell abundance level group. For many typical cell sample comparisons then, the JDA for each compared cell sample is the same when the SCR=1, and the just detectable abundance level for the HCN is 3 copies per cell for the assay. This assay situation approximates many prior art assays. Because of the large pool of unregulated or nearly unregulated genes which exist for each typical mammalian cell sample comparison, the different assay gene comparisons which are associated with detectable abundance values of 1-5 or so, will also be associated with a large number of unregulated or nearly unregulated low abundance genes. Thus, it appears that the fifth requirement is met for many mammalian genes for typical assays which have a just detectable abundance range which spans the 1-5 or so copies per cell range. Prior art reports a large number of these.

Requirements 1-5 for the certain occurrence of an EA Rule or SCR related false negative result and RDM, appear to be met for a large number of individual genes in prior art microarray and non-microarray gene comparisons. The discussion of the real life relevance of the sixth requirement will assume that requirements 1-5 have been met.

The sixth requirement specifies the following. The magnitude of the gene's assay SCR value deviation from one, and the resulting deviation of the gene's assay ACR from the T-DGER must be great enough, so that the gene's LCN sample mRNA abundance level is not detectable in the assay. This must occur even though, the gene's HCN sample mRNA abundance level is detectable in the same assay, and has an mRNA abundance level which is equal to or less than, the gene's LCN sample mRNA abundance level. The larger the assay SCR value deviation from one, the greater the magnitude of the deviation of the assay ACR from the T-DGER. The further a gene's assay ACR deviates from the T-DGER, the higher the gene's LCN sample mRNA abundance can be, and still be undetectable in the assay, and the greater the difference can be in the assay between the detectable HCN sample gene mRNA abundance, and the undetectable LCN sample gene mRNA abundance, and still get the occurrence of an EA Rule related false negative result for the gene in the LCN sample. As an example, if the deviation of a gene's T-DGER value from the ACR is twenty fold, and the deviation of the assay SCR from one, is twenty fold, an LCN sample mRNA abundance level of 99 mRNA copies per cell for the gene, will be undetectable in the assay, even though the HCN sample mRNA abundance level for the same gene in the same assay is 5 mRNA copies per cell, and is just detectable in the assay. Here, the LCN sample mRNA abundance level range over which an EA Rule related false negative result for the gene can occur, is 5-99 mRNA copies per cell. If in a gene comparison assay, the LCN sample mRNA abundance level for the gene is less than 5 copies per cell, or 100 or more copies per cell, an EA Rule related false negative cannot occur for the gene in the LCN sample. Whether the LCN sample's gene mRNA abundance level coincides with the 5-99 copies per cell abundance level range over which an EA Rule related false negative result for the gene will occur, depends on biological factors.

Given that requirements 1-5 are met for a large number of prior art prokaryotic and eukaryotic particular gene comparisons, the real life relevance of the sixth requirement hinges on the following. (i) Whether the magnitude of the deviation of the assay SCR from one, which commonly occurs in the prior art gene comparisons, is enough to allow the occurrence of EA Rule related false negative results. (ii) The number of different genes in a typical prior art gene comparison which are active in both cell samples, and which have an LCN sample mRNA abundance level which properly overlaps the mRNA abundance level in the LCN sample over which an EA related false negative result can occur for the gene in the LCN sample.

Significant differences in the total RNA content per cell, and the total mRNA content per cell, are common for different types of prokaryotic and eukaryotic cells. As discussed earlier, the amount of total RNA per mammalian cell can vary over a range of about 25 fold for different cell samples from one mammalian organism. The amount of total cytopasmic RNA obtained from different types of certain mammalian tissue culture cells can vary by 16 fold. Within a homogeneous population of one type of bacterial or mammalian cells, the total RNA content per cell can vary by 4 to 10 fold or more, depending on the physiological state of the cells. The total mRNA content per cell can also vary significantly in different prokaryotic and eukaryotic cell types. Different cell types from the same mammalian organism may vary in total mRNA content per cell by 10 fold or more. Within a homogeneous population of one type of bacterial or mammalian cell, the total mRNA content per cell can vary by up to 4-6 fold or more, depending on the physiological state of the cells. The available information on the relative total RNA or mRNA contents of cells, indicates that 2-10 fold differences are not uncommon. As discussed earlier, 4 to 10 fold differences in total RNA or total mRNA content per cell, can occur for the same bacterial or mammalian cells at different growth stages.

Prior art microarray gene comparison, and the non-microarray corroborative gene comparison practice, rarely if ever, determines the total RNA content per cell, or total mRNA content per cell, or both, for each of the cell samples compared. There is relatively little information available, concerning: the total mRNA per cell, or total mRNA transcripts per cell, for different cells and tissue types; or the effect of various physical and chemical treatments on the total RNA and/or total mRNA per cell content of different cells and tissue types. However, as discussed above, different cells and tissue types often have total RNA per cell, and/or total mRNA per cell amounts, which vary significantly. In addition, even within a homogeneous population of just one cell type, such as the earlier discussed mouse 3T3 tissue culture cells, 4-6 fold differences in the total RNA content per cell, and total mRNA content per exist. For those prior art cell sample comparisons for which no RNA per cell content information exists, it cannot be known whether the total RNA content per cell, and/or total mRNA content per cell, of the compared cell samples are the same or not. Therefore, it cannot be known whether the assay SCR=1, or not. However, for many prior art gene comparisons, the total RNA and total mRNA contents per cell are known to differ, and therefore for those gene comparisons, when the EA Rule is practiced the assay SCR value is known to not equal one. Further, it is likely that many of these prior art gene comparisons have assay SCR values which deviate from one by two to four fold or more. In this event, the assay ACR for a particular gene comparison will deviate from the gene's T-DGER by two to four fold.

Prior art does not determine, or take into consideration during the normalization of gene comparison results, the assay SCR. As discussed, the assay SCR or SCR is a global assay variable NF, and as such the SCR value affects all of the particular gene comparisons in a cell sample comparison in the same way. It is important to note that the prior art normalization process cannot correct the gene comparison results for the presence of prior art considered NF related false negative results, or the prior art unconsidered EA Rule or SCR related false negative results. Further, a normalization process which perfectly corrects the gene comparison results for all pertinent assay variables, also will not, and cannot, correct for the presence of any assay variable false negative result.

The above discussion indicates that for many prior art gene comparisons, the assay SCR value deviates from one by 2-4 fold, and that the deviation may be much greater for many other prior art particular gene comparisons. It is clear that such 2-4 fold deviations are large enough to cause EA Rule or SCR related false negative results and RDMs, if all six requirements are met. Table 36 illustrates this. Table 36 illustrates that SCR related false negative results occur only when the LCN sample Gene A mRNA abundance level properly overlaps with the Gene A mRNA abundance level range in the LCN sample, (see Table 36 i-iv, and vi-viii).

TABLE 36
Occurrence of Assay SCR Related False Negative Results in LCN Sample

| | | | Gene's Just | | | | Gene's Just | | Occurence |
|---|---|---|---|---|---|---|---|---|---|
| | | | Detectable | Gene's | Gene's | | Detectable | | of SCR |
| | | | HCN Cell | HCN Cell | LCN Cell | | LCN Cell | | Related |
| | | | mRNA | mRNA | mRNA | | mRNA | | False |
| | | | Abundance | abundance | Abundance | | Abundance | Detectability | Negative |
| | | | Level in | Level for | Level for | Assay SCR | Level in | of Gene | Result for |
| Gene | | Cell Sample | Assay | Assay | Assay | Deviation | Assay | Activity in | Gene in |
| Compared | | Compared | (CPC)^{(a)} | (CPC) | (CPC) | From One | (CPC) | Assay | LCN |
| (i) | A | HCN | 3 | 3 | | 2 | | YES | |
| | A | LCN | | | 3 | | 6^{(b)} | NO | YES |
| (ii) | A | HCN | 300 | 300 | | 2 | | YES | |
| | A | LCN | | | 300 | | 600 | NO | YES |
| (iii) | A | HCN | 3 | 3 | | | | YES | |
| | A | LCN | | | 5.9 | 2 | 6 | NO | YES |
| (iv) | A | HCN | 300 | 300 | | 2 | | YES | |
| | A | LCN | | | 400 | | 600 | NO | YES |
| (v) | A | HCN | 3 | 3 | | 4 | | YES | |
| | A | LCN | | | 12 | | 12 | YES | NO |
| (vi) | A | HCN | 3 | 3 | | 4 | | YES | |
| | A | LCN | | | 11.9 | | 12 | NO | YES |
| (vii) | A | HCN | 10^{(b)} | 40 | | 4 | | YES | |
| | A | LCN | | | 39 | | 40^{(b)} | NO | YES |
| (viii) | A | HCN | 3 | 3 | | 20 | | YES | |
| | A | LCN | | | 59 | | 60 | NO | YES |

^{(a)}CPC = mRNA copies per cell.
^{(b)}LCN sample mRNA abundance level over which SCR related false negative results can occur. For (i) the range is 3 to <6 Gene A CPC. For (vii) the range is 10 to <40 Gene A CPC. For (viii) the range is 3 to <60 Gene A CPC.

The incidence of occurrence of these SCR related false negative results in typical prior art microarray and non-microarray gene comparisons depends upon, the number of LCN sample active in both cell samples genes present in such a gene comparison assay which have mRNA abundance levels which coincide with the LCN sample mRNA abundance level over which such false negative results can occur. The magnitude of this gene number in prior art gene comparisons, is discussed below.

As illustrated in Table 36, SCR related false negatives and RDMs can occur at high or low abundance levels. For a typical prior art gene comparison, the number of active in both cell samples genes which have a high mRNA abundance level is relatively small. In mammals, the medium and high abundance genes comprise roughly 5-10 percent of the total number of expressed genes. The incidence of occurrence of SCR related false negatives for these medium and high abundance genes will be relatively small due to the small numbers involved. In contrast, it has been estimated that about 0.85 of the expressed genes in mammalian cell samples, or roughly 10,000 genes, have a mRNA abundance level of 1-5 mRNA copies per cell. As discussed earlier, for a typical mammalian cell sample comparison, about 5,000 or so of the same 1-5 copy per cell genes, are actively expressed in both cell samples. In addition, the mRNA abundance of a particular active 1-5 copy per cell low abundance gene in one cell sample, is similar to or equal to, the mRNA abundance level of the same 1-5 copy per cell low abundance gene, present in the other cell sample. Prior art believes that generally, only a small number of these active in both cell samples 1-5 copy per cell low mRNA abundance genes, are differentially expressed. For those active in each cell sample 1-5 copy per cell low mRNA abundance genes which are differentially expressed, the maximum T-DGER=5, and it is likely that most of these genes will differ in expression by 2-3 fold. Prior art also commonly practices that for a typical mammalian cell sample gene comparison, the great majority of those 1-5 copy per cell low mRNA abundance genes which are active in both cell samples, or about 4,000-5,000 genes, are unregulated, and have a T-DGER=1. For a typical prior art mammalian cell sample gene comparison, each of these 4,000-5,000 unregulated 1-5 copy per cell low abundance genes, meets requirements 1-5. The potential incidence of occurrence of EA Rule or SCR related false negative results and RDMs, in prior art mammalian gene comparison practice is evaluated below.

As discussed, many prior art microarray and non-microarray gene comparisons have assay SCR values which deviate from one by two to four fold. It is not uncommon for a prior art microarray mammalian cell sample gene comparison assay, to have a HCN sample just detectable mRNA abundance level of 3-10 mRNA copies per cell. Here for simplicity, the following will be assumed for this discussion on the incidence of SCR related false negative results in prior art particular gene comparison assays. (a) The HCN sample just detectable mRNA abundance level, is 3 copies per cell for each of the different 5,000 or so unregulated 1-5 copy per cell low mRNA abundance level genes. (b) The magnitude of the deviation of each gene's assay SCR value from one, is two to four fold. This situation is illustrated in Table 36. Table 36 (i) (iii) indicate for this situation that when a gene's SCR deviation is 2 fold, then the LCN sample mRNA abundance level range over which a SCR related false negative will occur, is from 3 to almost 6 mRNA copies per cell. Here, the HCN sample's just detectable mRNA abundance level of 3 copies per cell, closely coincides with the 3˜6 copy per cell HCN sample mRNA abundance level, over which an SCR related false negative can occur for the LCN sample 1-5 copy per cell low mRNA abundance level genes. Here, of the 4,000-5,000, 1-5 copy per cell low mRNA abundance LCN sample genes, the ones which have an LCN sample mRNA abundance level of 3 to about 6 mRNA copies per cell will not be detected in the assay, and therefore will be associated with SCR related false negative results and RDMs. This LCN sample mRNA abundance level range of 3 to about 6 CPC represents about 0.4 of the 1-5 copy per cell low cell mRNA abundance level range, which comprises 4,000-5,000 different mammalian active genes. It is not known how many LCN sample genes are actually present, in this 3˜6 copy per cell region of the LCN low mRNA abundance level genes. However, if it is assumed that the genes are evenly distributed over the 1-5 copy per cell range, the number of SCR related false negative results which will occur in this typical mammalian cell sample gene comparison assay, is roughly 1,600. In the above assay situation, if the assay SCR deviates from one by 4 fold, the LCN sample mRNA abundance level over which an SCR related false negative result can occur, ranges from 3 to almost 12 copies per cell (see Table 36 v, vi). In this event, nearly half of the 4,000-5,000 LCN sample low mRNA abundance genes can be associated with SCR related false negative results.

As discussed above, for a typical prior art microarray cell sample gene comparison, the HCN sample is associated with a large number of low mRNA abundance level 1-5 mRNA copy per cell genes, which are detectable as active only in the HCN sample. Each of these HCN sample active genes is not detectable or inactive in the assays LCN sample. In a high density microarray mammalian cell comparison the number of genes in each of the said, active only in the HCN sample, and inactive in the LCN sample, categories can be hundreds to thousands. For a cell sample gene comparison, many of the same inactive or undetected genes in the LCN sample, which are active in the HCN sample, may in fact be active, and meet the sixth requirement.

The above discussed considerations indicate that the sixth requirement is met for a large fraction of LCN sample 1-5 copy per cell low abundance genes under certain, not uncommon, prior art assay conditions used for mammalian cell sample gene comparisons. While the above discussion has focused on whether the sixth requirement was met for a significant number of prior art mammalian LCN sample 1-5 copy per cell unregulated low abundance mRNA genes, the discussion and conclusions also apply to the differentially expressed 1-5 copy per cell LCN sample low mRNA abundance level genes, as well as to both unregulated and differentially expressed genes in the LCN sample, which have a mRNA abundance above 5 copies per cell. The discussion and conclusions also apply to many prior art non-mammalian eukaryotic and prokaryotic gene comparison LCN sample high, medium, or low mRNA abundance genes. With regard to LCN sample low mRNA abundance genes, in both eukaryotes and prokaryotes a large number of active in both compared cell sample genes have mRNA abundance levels of 1-5 mRNA copies per cell in both compared cell samples, and are believed by the prior art to be unregulated. Under certain, not uncommon, prior art assay conditions used for eukaryotic and prokaryotic cell sample gene comparisons, a significant fraction of these LCN sample 1-5 copy per cell low mRNA abundance genes will be associated with SCR related false negative results.

EA Rule or SCR related false negative results and their associated RDMs can also occur for DGDS particular gene comparisons, and under certain circumstances, can also occur for DGSS particular gene comparisons. The above discussion applies directly to these DGDS and DGSS comparison assays.

Interpretation of EA Rule and (ACR≠T-DGER) Related False Negative Results.

These EA Rule related false negative gene activity results cannot occur when either, the number of sample cells compared is equal, or enough sample RNA is added to the assay to ensure the detection of the least abundant mRNA in each sample being compared. Neither of these conditions is often met in mammalian gene activity comparisons. For prior art prokaryote and simple eukaryote gene activity comparisons the first condition is often not met, that is the EA Rule is practiced. The second condition, while rarely met, is approximately met much more often for prokaryotes and simple eukaryotes, than for mammals. The consequence of not meeting one or the other of these conditions is summarized below.

A typical prior art mammalian gene activity comparison practices the EA Rule and does not involve enough sample mRNA to ensure that every mRNA type present in each sample, including all low abundance mRNAs, is detectable in the assay. In such a comparison, when a positive result associated with a relatively low assay signal is obtained for a particular gene in the HCN sample, and a negative result is obtained for the same gene in the LCN sample, the interpretation of the LCN sample negative result is uncertain. The LCN sample negative result, is caused by one of three different situations which might exist in the LCN sample. First, the gene is inactive in the LCN sample, and thus the negative result is a true negative result. In this case, an interpretation that relative to the HCN sample gene, the LCN sample gene is downregulated would be correct. Second, the LCN sample gene is active, but not active enough to be detected, even if the number of LCN sample cells compared is increased so that equal numbers of HCN sample cells and LCN sample cells are compared. This situation produces a false negative result. This type of false negative result was earlier termed a non-EA Rule related false negative result. In this second case, an interpretation that relative to the HCN sample gene, the LCN sample gene is down-regulated would be correct. Third, on a per cell basis, the activity of the LCN sample gene is equal to or greater than the activity of the same gene in the HCN sample, and because of the practice of the EA Rule this situation produces a false negative result, herein termed an EA Rule or SCR related false negative result. In this third case, an interpretation that relative to the HCN sample gene, the LCN sample gene is downregulated, is incorrect.

For a particular prior art gene comparison, where a positive result for the gene in one cell sample is associated with a relatively low assay signal, and a negative result is obtained for the same gene in the other cell sample, the interpretation of the negative result is uncertain. In reality, the negative cell sample gene could be active or inactive. In addition, the interpretation of the direction of gene regulation differences between the inactive gene in one cell sample, and the active gene in the other cell sample, is also uncertain. In reality, relative to the active gene in the one cell sample, the negative gene in the other cell sample could be upregulated, downregulated, or unregulated. Absent some knowledge of the assay SCR value, and the gene's cell sample mRNA abundance level range over which such EA Rule or SCR related false negatives can occur in the assay, the interpretation of a negative result for a gene in this situation is uncertain. Prior art practice for microarray gene comparisons, and non-microarray corroborative gene comparisons, does not determine the assay SCR, or mRNA abundance level range over which such EA Rule or SCR false negatives can occur. In addition, prior art gene comparison assays rarely involve enough cell sample mRNA, or equivalents, in the assay, to ensure the detection of the least abundant mRNA in each cell sample being compared. Thus, for such a prior art situation where, a positive gene activity result for a gene in one cell sample is associated with a relatively low assay signal, and a negative gene activity result is obtained for the same gene in a different cell sample, the interpretation of the negative result is uncertain. Note that if the deviation from one of the assay SCR value is large enough, the positive assay result associated with an SCR related false negative can be quite large.

Deviations from the Ea Rule in Prior Art Microarray and Non-Microarray Practice.

Up to this point it has been assumed that the EA Rule has been practiced in an ideal fashion in the context of the current microarray assay analysis. The ideal practice of the EA Rule requires that it must be known that the microarray hybridization solution actually contains equal masses of total RNA or total mRNA, or equivalents, from each sample to be compared. In standard microarray practice, the EA Rule generally has not been practiced ideally. The reality of the current microarray practice is that the usual microarray hybridization solution is put together in a way that often makes it difficult, if not impossible, to know whether it contains equal masses of total RNA or total mRNA cDNA or cRNA equivalents from each sample. Only rarely is the natural total cell RNA, or total cell mRNA, added directly to the microarray hybridization solution. Instead, the natural RNA is converted to an equivalent form, which is then added to the hybridization solution. The commonest equivalent form is complementary DNA (cDNA), which is produced by copying the natural total RNA, or total mRNA, with reverse transcriptase.

A second equivalent form in use is the complementary RNA (cRNA), which is produced by a complex process where: the RNA is converted to first strand cDNA; the first strand cDNA is then converted to double strand by producing the second strand cDNA which also incorporates a T7 polymerase promoter; then using this double strand form to produce cRNA. The cDNA and cRNA molecules are labeled during the production process. This labeled cDNA or cRNA is then added to the hybridization solution.

In standard microarray practice for producing the equivalent form, the EA Rule is usually used and the same amount of total RNA or mRNA from each sample to be compared is used to produce the cDNA or cRNA. However, the amount of cDNA yield from each sample's RNA and the amount of cDNA from each sample which is added to the hybridization solution, is very rarely reported. Whether these measurements were done, and just not reported, is not known. The situation for cRNA is somewhat better, and these amounts are reported more often. It seems likely that, the amount of each samples RNA equivalents which is present in the microarray hybridization solution, is not known for many if not most microarray analyzes. Thus, it is not known whether the EA Rule is being practiced for most microarray assays. The uncertainties involved with these various deviations further contribute to the uninterpretability of the EA Rule related N-DGER generated by standard microarray practice. This makes it more difficult to derive a T-DGER for the natural RNA comparison. As discussed in the previous section the use of a housekeeping gene mRNA as an internal control does not help clarify the interpretation.

The above discussion applies directly to the non-microarray method gene expression analysis methods, including RT-PCR.

Occurrence of False Negative Gene Activity Results and Regulation Direction Miscalls (RDMs) Associated with (ACR≠RASR).

The nature of gene activity comparison true positive and false negative results, is discussed earlier. In the context of that discussion there are two kinds of assay false negative results. The first is termed a non-NF related false negative result. Here, the false negative result is associated with a correct gene regulation direction call, which indicates that the inactive gene in one cell sample is downregulated, relative to the active gene in the other cell sample. The second kind, termed an NF related false negative result, is associated with an RDM for the particular gene comparison. An NF related false negative result indicates that the inactive gene in one cell sample is downregulated, relative to the active gene in the other cell sample, when, in reality, in the compared cell samples the gene is unregulated, or upregulated.

Several different types of NF related false negative results and associated RDM's, can occur for a particular microarray or non-microarray gene comparison assay. One of these types is an NF related false negative result, which is related to only the ARR or SCR assay NF values. Herein, this NF related false negative result type is termed an EA Rule or SCR related false negative result. A second type, is an NF related false negative result which is related only to one or more, of the set of prior art considered and prior art unconsidered NF assay values, which does not includes but is not limited to, the NFs SCR and ARR. The set of prior art considered, and prior art unconsidered NFs includes, the C-HKR, spatial, print tip, print plate, intensity, PAFR, MLDR, PL-HKR, PS-HKR, PSAR, PSSR, LLSR, SBNR, and SSAR. This second type of NF related false negative result is termed a non-SCR related false negative result, or an NS false negative result. A third type of NF related false negative result is related to one or more of the NFs which are associated with the SCR related false negative results, and is also related to one or more of the NFs which are associated with the NS related false negative results. Herein, this third type is termed the mixed type NF related false negative results, or MT related false negative results.

The different NF related false negative types occur for different reasons. The first type, the SCR related false negative results, occurs because the (ACR)≠(T-DGER), for a particular gene comparison. The second type, the NS related false negative result, occurs because the (assay RASR)≠(ACR), for a particular gene comparison. The third type, the MT related false negative result, occurs because the (assay RASR)≠(ACR), and the (ACR)≠(T-DGER), for the particular gene comparison. All three of these NF related false negative types are associated with RDM's.

For a particular gene comparison, absent some assay variable or bias which affects the assay RASR the (assay RASR)=(ACR). However, when a particular gene comparison is associated with one or more assay variable or biases, which cause the assay RASR value to deviate from the assay ACR value, the (assay RASR)≠(ACR), and an NS or MT related false negative result can occur for the particular gene comparison. Thus, an NS or MT related false negative can occur for a particular gene comparison, whenever an assay RASR value must be normalized so that it equals the ACR present in the assay hybridization solution. As discussed, prior art believes that assay variables exist which cause the assay RASR value for a particular gene comparison to deviate from the ACR value in the assay hybridization solution for that particular gene comparison. Further, prior art believes that the assay RASR result for each particular gene comparison must be normalized or corrected for prior art known assay variables and biases, and that the resulting NASR value for each particular gene comparison is equal to the ACR for the gene comparison in the assay hybridization solution. Prior art believes that such assay RASR normalization is necessary in order to obtain biologically meaningful and interpretable gene comparison results. This indicates that prior art believes and practices that almost all prior art produced gene comparison assay RASR values are not equal to the ACR for the gene comparison. Thus, under one or another assay condition, most, if not all, of these prior art gene comparisons, have the potential to be associated with an NS or MT related false negative result. The prior art known and considered NFs, which can cause the (assay RASR)≠(ACR) for a particular gene comparison, are C-HKR, spatial, print tip, print plate, intensity, and AE•AER. The NFs, which are not considered by the prior art, and which can cause the (assay RASR)≠(ACR) for a particular gene comparison, include but are not limited to, the NS-UNF assay variables MLDR, PL-HKR, PS-HKR, PSAR, PSSR, LLSR, SBNR, SSAR. The prior art considered NF ARR, and the prior art unconsidered NF SCR, do not cause the assay RASR value to deviate from the ACR value.

Because the SCR related false negative results are associated only with the global NFs SCR and ARR, all particular gene comparisons in a microarray or non-microarray gene comparison assay have the same assay SCR value, and the same assay ARR value. In contrast, the product of all of the assay variable NFs pertinent to the particular gene comparison, may not be the same for each different particular gene comparison in the assay. Herein, the product of all of the assay variable prior art considered and unconsidered NFs, which are pertinent to a particular gene comparison, is termed the pertinent NF product, or PNFP. Both global and non-global NFs can be associated with a particular assay PNFP value for a particular gene comparison. As a consequence, in one gene expression analysis assay, different particular gene comparisons can have different assay PNFP values. NS and MT related false negative results may be associated with only global NFs, only non-global NFs, or a mixture of global and non-global NFs. When the NS related and MT related false negative results in an assay are associated with only global NFs, all particular gene comparisons in a cell sample gene comparison assay, have the same assay values for a particular global NF, and the PNFP. When the NS related or MT related false negative results are associated with only non-global NFs, different particular gene comparisons in the cell sample gene comparison assay, can have different assay values for a particular non-global NF, and for the PNFP. When NS related or MT related false negative results are associated with both global NFs, and non-global NFs, all particular gene comparisons in a cell sample gene comparison assay have the same assay values for a particular global NF, and in addition, different particular gene comparisons in the assay, can have different assay values for a particular non-global NF, and for the PNFP.

While different types of NF related false negative results are caused by different situations, the general characteristics of all NF related false negative results are essentially the same. An earlier section presents an extensive discussion of EA Rule or SCR related false negative results. While the discussion is presented in terms of SCR related false negative results, the discussion's general interpretations, and conclusions, apply directly to NS and MT false negative results, and associated RDMs. Such discussion, interpretations, and conclusions, apply directly to: the occurrence of NF related false negative results in general; the biological and assay conditions which favor the occurrence of NF related false negative results in general; the role of the assay JDQ for particular gene mRNA LPN molecules in the occurrence of NF related false negatives in general; the identification for a microarray or non-microarray gene expression analysis assay, of the mRNA abundance levels and range over which NF related false negative results may occur; the likelihood that significant numbers of NF related false negative results in general have occurred in prior art microarray and non-microarray gene comparison assays; the prior art interpretation of NF related false negatives in general.

This current discussion concerns the occurrence of particular gene comparison NF related false negative results, which are the result of an assay situation where the (assay RASR)≠(ACR). Such false negative results are associated with NS and MT false negative results. For this discussion SGDS comparisons of particular gene mRNA transcripts will be emphasized. However, the discussion applies directly to all SGDS, DGDS, and DGSS comparisons of viral, prokaryotic, eukaryotic, and standard RNA transcripts of all kinds. This includes all types of rRNA, tRNA, mRNA, siRNA, miRNA, snoRNA, antisense RNA, and other known and unknown RNAs.

It will be useful for this discussion to consider the earlier extensive discussion on the just detectable amount (JDQ), of a particular cell sample mRNA LPN in a gene comparison assay, and the JDQR of the assay compared cell sample mRNA LPN molecules in a gene comparison assay. This discussion on the assay JDQ and JDQR is directly applicable to this current discussion. It will also be useful to consider the earlier discussion concerning the effect of the assay NFs PSAR, and LLSR, on the relationship, (assay RASR)=(ACR). This discussion is also directly applicable to the current discussion.

An NF related false negative result for a particular gene comparison, which is associated with a situation where, the (assay RASR)≠(ACR), can occur, when the (assay JDQR)≠1, or when the assay signal activity ratio of the compared particular mRNA LPN molecules≠1, or when both these situations occur. Herein, the assay signal activity ratio of the compared particular mRNA LPN molecules, is termed the assay signal activity ratio, or assay SAR. The assay SAR for a particular SGDS Type 1 mRNA LPN gene comparison, is influenced by the TSAR of the compared cell sample mRNA LPN preparations, but is not necessarily equal to the TSAR. The SAR for a particular SGDS Type 1 mRNA LPN gene comparison is equal to the assay PSAR for that particular gene comparison. The SAR for a particular SGDS Type 2 mRNA LPN gene comparison is equal to the assay LLSR for that particular gene comparison. In other words, for a particular gene comparison, an NS or MT related false negative result can occur when; the (assay JDQR)=1, and the (assay SAR≠1); or when the (assay JDQR)≠1, and the (assay SAR)=1; or when the (assay JDQR)≠1, and the (assay SAR)≠1. For a particular gene comparison, the farther the assay JDQR or the assay SAR, or the product of the assay JDQR and SAR, deviates from one, the greater the opportunity for the occurrence of an NS or MT related false negative result and RDM. Note that when a sufficient amount of each compared cell sample's mRNA LPN preparation is added to the gene expression analysis assay to ensure that every different particular mRNA LPN which is in the assay hybridization solution is present in a detectable amount, NF related false negative results cannot occur. Note also that when all the pertinent assay NF values for a particular gene comparison equal one, an SCR, NS, or MT related false negative cannot occur. Here, the assay PNFP=1. However, for a particular gene comparison, having a PNFP=1, does not guarantee that an NF related false negative cannot occur. Note further, that neither the assay SCR, or assay ARR, influences the assay value for the JDQR or SAR.

Assay variable NFs, which can cause the assay JDQR not to equal one, include those CNFs, which are considered for the prior art normalization of assay RASR values, and UNFs, which are not considered for the prior art normalization of assay RASR values. Prior art considered NFs include, TSAR, C-HKR, spatial, print tip, print plate, intensity, scale, AE•SER, AE•AER. Assay variable NFs not considered by the prior art include, MLDR, PL-HKR, PS-HKR, PSAR, PSSR, LLSR, SBNR, SSAR. For a particular gene comparison, a JDAR≠1 occurs when a difference in the hybridization kinetics of each compared particular gene mRNA LPN molecule population with the particular gene complementary detection polynucleotides (CDP), exists for the particular gene comparison. Note that a difference in the signal activity of the compared particular gene LPN molecules, can result in a difference in the hybridization kinetics of the compared particular gene mRNA LPN molecules with the particular gene CDP. The assay variable NFs, spatial print tip, print plate, can also influence these hybridization kinetics, and can therefore, cause the assay JDQR≠1. Generally, in a gene comparison assay, the particular gene mRNA LPN associated with the faster hybridization kinetics, and/or the higher label signal activity, has the lowest assay JDQ.

Assay variable NFs, which can cause the assay SAR not to equal one, include but are not limited to, the prior art considered TSAR, and UNFs MLDR, PSAR, PSSR, and LLSR. For a particular gene comparison, when the assay SAR≠1, the hybridization of each different cell sample particular gene LPN to the gene CDP, results in a quantity of signal label activity being associated with a single hybridized CDP molecule, which is different for one cell sample's particular gene mRNA LPN, than for the other compared cell sample's particular gene mRNA LPN.

An NF related false negative result for a particular gene comparison which is the result of the (assay RASR)≠(ACR), can occur when the assay JDQR≠1. When for a particular gene comparison, the assay JDQR≠1, an assay situation exists where, the assay value for the JDQ of the particular gene mRNA LPN molecules from one cell sample, is higher than the assay value of the JDQ of the other cell sample's particular gene mRNA LPN molecules. For simplicity herein, for a particular gene comparison, the cell sample which is associated with the higher assay JDQ value, is termed the high JDQ cell sample, or HJDS, while the cell sample which is associated with the lower assay JDQ value, is termed the low JDQ cell sample, or UDS. The particular gene mRNA LPN from the UDS, will because of a faster hybridization rate, or greater signal activity or both, associate more signal activity with the particular gene CDP, than the particular gene mRNA LPN from the HJDS. Consequently, the assay RASR value obtained for the particular gene comparison, will be disproportionally enriched for the label signal activity from the UDS LPN, and therefore not equal the ACR. Note that the HJDS here is analogous to the low cell number cell sample (LCN), associated with SCR related false negative results, and the UDS is analogous to the high cell number cell sample (HCN).

An NF related false negative result for a particular gene comparison, which is the result of the (assay RASR)≠(ACR), can occur when the assay SAR≠1. When for a particular gene comparison, the assay SAR≠1, an assay situation exists where the signal activity of the particular mRNA LPN molecules representing one cell sample, is higher than the signal activity of the same particular mRNA LPN molecules representing the other compared cell sample. Herein, the cell sample associated with the high signal activity LPN is termed the high signal activity cell sample, or HSAS, while the other cell sample is termed the low signal activity cell sample, or LSAS. When the assay SAR≠1, for a particular gene comparisons, the quantity of signal label associated with a single particular gene CDP molecule, can be greater for the HSAS particular gene LPN, than for the LSAS particular gene LPN. This can occur even when the assay JDQR=1. As a result, the (assay RASR)≠(ACR).

An NS related false negative result for a particular gene comparison where the (assay RASR)≠(ACR), is certain to occur under the following conditions. First, the particular gene of interest must be active in each compared cell sample. Second, as a result of the assay JDQR and/or assay SAR values, the (assay RASR)≠(ACR). Third, the particular gene's mRNA abundance level in the LJDS or HSAS, must be equal to or less than, the same gene's mRNA abundance level in the HJDS or LSAS. Therefore, the particular gene's mRNA abundance level in the HJDS or LSAS, must be equal to or greater than, the same gene's mRNA abundance level in the UDS or HSAS. Fourth, a detectable amount of the particular gene mRNA LPN from the LJDS or HSAS, must be present in the assay hybridization solution. Put differently, the particular gene's mRNA abundance level in the UDS or HSAS, must be detectable in the assay. Fifth, for the particular gene comparison, the magnitude of the deviation of the gene's assay JDQR and/or assay SAR from one, and the resulting deviation of the genes assay RASR value from the ACR, must be great enough so that the gene's mRNA abundance in the HJDS or LSAS, is not detectable in the assay, even though the gene's HJDS or LSAS mRNA abundance level is equal to or greater than, the gene's UDS or HSAS mRNA abundance level in the same assay.

The occurrence of an NS related false negative result and RDM, can be illustrated by considering an idealized gene comparison assay. For such an idealized assay, the following is assumed. Gene A is actively expressed in Cell Samples 1 and 2. As a result of the assay JDQR and/or SAR values, the (assay RASR)≠(ACR) for the gene A comparison. The UDS or HSAS gene A mRNA abundance level, is equal to or less than the HJDS or LSAS gene A mRNA abundance level, and the HJDS or LSAS gene A mRNA abundance level, is equal to or greater than the LJDS or HSAS gene A mRNA abundance level. The LJDS or HSAS gene A mRNA abundance level is known, and is detectable in the assay. The HJDS or LSAS gene A mRNA abundance level is known, and is not detectable in the assay because of the effect of the assay JDQR and/or SAR values on the deviation of the gene A assay RASR from the ACR. The magnitude of the deviation of the gene A assay JDQR or SAR value from one, is equal to the magnitude of the deviation of the gene A assay RASR from the ACR. Only the JDQR and/or SAR NFs affects the gene A assay RASR value, and all other pertinent assay variable NFs have an assay value of one, including the SCR. Cell Sample 1 is always the LJDS or LSAS. For simplicity the illustration is primarily presented in terms of the JDQR. However, the results and conclusions are also directly applicable to the SAR. Note that both JDQR and SAR related false negative results are NS NF related false negative results.

Table 37A & B (which together represent one table) summarizes the illustrations. Table 37A & B (i) and (vi) illustrate that the activity of gene A in the HJDS is not detected in the assay, even though the SCR=1, and the HJDS mRNA abundance level is equal to the gene A UDS mRNA abundance level, which is detectable in the same assay. This occurs because the gene A assay JDQR value of 0.91, causes the gene A assay RASR to deviate from the ACR enough so that the gene A activity in the HJDS is not detected in the assay. The result is a JDQR related false negative result for gene A in the HJDS. The illustrations of Table 37A & B (ii) (iv) (v) (vii) (viii), indicate that the activity of gene A in the HJDS is not detected in the assay, even though gene A has a higher cell mRNA abundance in the HJDS, than the UDS, and the SCR=1.

TABLE 37A
Occurrence of Gene A NF Related False Negative Results Associated
with the (Assay RASR) ≠ (ACR)

| | | Gene A Just | | | |
|---|---|---|---|---|---|
| | | Detectable | Gene A | | Gene A |
| | | LJDS mRNA | mRNA | Gene A | mRNA |
| | | Level in | Abundance | mRNA | LPN |
| Cell Sample | | Assay | Level in | LPN Assay | Assay |
| Compared | | (CPC)^{(b)} | Assay (CPC) | JDQR^{(c)} | SAR |
| (i) | ^{(d)}LJDS^{(a)} | 4 | 4 | 0.91 | 1 |
| | HJDS | | 4 | | |
| (ii) | LJDS | 200 | 200 | 0.3 | 1 |
| | HJDS | | 600 | | |
| (iii) | LJDS | 4 | 4 | 0.5 | 1 |
| | HJDS | | 8 | | |
| (iv) | LJDS | 4 | 4 | 0.4 | 1 |
| | HJDS | | 8 | | |
| (v) | LJDS | 2 | 2 | 0.3 | 1 |
| | HJDS | | 6 | | |
| (vi) | LJDS | 4 | 4 | 1 | 1.1 |
| | HJDS | | 4 | | |
| (vii) | LJDS | 2 | 2 | 1 | 3.3 |
| | HJDS | | 6 | | |
| (viii) | LJDS | 4 | 4 | 0.4 | 2.25 |
| | HJDS | | 20 | | |

^{(a)}LJDS is always Cell Sample 1.
^{(b)}CPC = mRNA copies per cell.
^{(c)}All ratios have Cell Sample 1 parameters in numerator.
^{(d)}The assay SCR = 1 for all examples.
^{(e)}(0.8) = (2.5 × 4) ÷ (1 × 8).

TABLE 37B
Occurrence of Gene A NF Related False Negative Results Associated
with the (Assay RASR) ≠ (ACR)

| | | | | | Occur- |
|---|---|---|---|---|---|
| | | | | Detect- | rence of |
| | | | Just | ability | HJDS |
| | | mRNA LPN | Detectable | of Cell | Gene A |
| | | Signal Activity | Gene A | Sample | NS NF |
| | | Associated | LPN Signal | Gene | Related |
| Cell Sample | | with Gene A | Activity in | A LPN in | False |
| Compared | | CDP | Assay | Assay | Negative |
| (i) | ^{(d)}LJDS^{(a)} | 1 | 1 | + | No |
| | HJDS | 0.91 | 1 | − | Yes |
| (ii) | LJDS | 1 | 1 | + | No |
| | HJDS | 0.91 | 1 | − | Yes |
| (iii) | LJDS | 1 | 1 | + | No |
| | HJDS | 1 | 1 | + | No |
| (iv) | LJDS | 1 | 1 | + | No |
| | HJDS | 0.8^{(e)} | 1 | − | Yes |
| (v) | LJDS | 1 | 1 | + | No |
| | HJDS | 0.91 | 1 | − | Yes |
| (vi) | LJDS | 1 | 1 | + | No |
| | HJDS | 0.91 | 1 | − | Yes |
| (vii) | LJDS | 1 | 1 | + | No |
| | HJDS | 0.9 | 1 | − | Yes |
| (viii) | LJDS | 1 | 1 | + | No |
| | HJDS | 0.9 | 1 | − | Yes |

^{(a)}LJDS is always Cell Sample 1.
^{(b)}CPC = mRNA copies per cell.
^{(c)}All ratios have Cell Sample 1 parameters in numerator.
^{(d)}The assay SCR = 1 for all examples.
^{(e)}(0.8) = (2.5 × 4) ÷ (1 × 8).

The result is a JDQR related false negative result for gene A in the HJDS. Table 37A & B (iii) illustrates that the JDQR related false negative results do not always occur when the gene A assay JDQR≠1, and Table 37A & B (iv) illustrates that when the gene A assay JDQR deviates sufficiently from one, a gene A JDQR related, and (assay RASR)≠(ACR) related, false negative result can occur. For Table 37A & B (iii), the JDQR=0.5, but does not deviate from one by enough to cause a JDQR related false negative result for gene

A. For Table 37A & B (iv) the JDQR=0.4, but does deviate from one enough to cause a JDQR related false negative result for gene A. The illustrations of Table 37A & B also indicate that the greater the deviation of the assay JDQR, or the assay SAR, or the product of the assay JDQR and the assay SAR, from one, the greater the mRNA abundance range over which the gene A NF related false negatives and RDM's, can occur. Further, the illustrations indicate that, depending on the assay just detectable mRNA abundance level, these false negative results and RDM's can occur for any mRNA abundance level in a cell sample. As discussed, the assay value for JDQR can be influenced by most, if not all, of the prior art considered and not considered assay variable NFs, while the assay values for the NFs TSAR, PSAR, and LLSR, affect the SAR, as well as the JDQR assay values. The scenarios illustrated in Table 37A & B, represent only a very small fraction of the possible microarray and non-microarray assay situations, where (assay RASR)≠(ACR) related false negative results and RDMs, can occur. Note that, while EA Rule or SCR related false negative results cannot occur when the assay SCR=1, the NF related false negative associated with the (assay RASR)≠(ACR), can occur when the SCR=1. However, said NF related false negative results and RDMs, cannot occur when all of the pertinent assay variable NFs equal one.

The above illustrations and discussion establish that (assay RASR)≠(ACR) related false negative results and RDMs can occur under certain microarray and non-microarray assay conditions, and not under other such assay conditions. The relevance of these NS related false negative results to prior art microarray and non-microarray gene comparison assays is discussed below.

Do (Assay RASR)≠(ACR) Related False Negative Results Occur in Real Life?

As discussed, an (assay RASR)≠(ACR) associated NS related false negative result is certain to occur in real life microarray gene comparisons, if all five of the earlier described conditions are met for one or more particular gene comparisons. The following discussion will examine the extent to which each required condition met in prior art microarray and non-microarray gene comparison practice. Note that (assay RASR)≠(ACR) associated NS related false negative results can be caused by prior art considered NFs, or prior art unconsidered NFs, or both.

As discussed earlier, prior art microarray and non-microarray practice does not determine and take into consideration the assay SCR value. Consequently, the assay SCR value for any particular prior art microarray gene comparison assay, is unknown. This complicates the evaluation of whether a significant number of (assay RASR)≠(ACR) related false negatives occur in the prior art microarray practice, since when the assay SCR≠1, SCR related false negative results can occur. For simplification, it will be assumed for this current discussion, that the assay SCR=1 for all prior art gene comparison assays, even though it clearly doesn't. Note that, for a particular prior art gene comparison, if both the assay SCR≠1, and the (assay RASR)≠(ACR), the opportunity for the occurrence of an NF related false negative result generally increases. Thus, an estimate of the likelihood of occurrence of prior art (assay RASR)≠(ACR), related false negative results and RDMs, will underestimate the likelihood of occurrence of prior art NF related false negatives and RDMs in general.

Another complication for this discussion is the effect of the interaction of the assay JDQR value and the assay SAR value, for a particular gene comparison, on the relationship (assay RASR)=(ACR). Under certain assay conditions, the product of the assay JDQR and assay SAR values may effectively equal one. In this event the (assay RASR)=(ACR), and an NS related false negative will not occur. This will occur only rarely in real life. For other assay conditions, when for a particular gene comparison, the assay JDQR value is greater than one, and the assay SAR value is less than one, the product of the assay JDQR and assay SAR values will not be equal to one, and will be dominated by the JDQR value, or the SAR value. In the event that the product is dominated by the JDQR value, the cell sample which is associated with the lower JDQ value is effectively the UDS. Alternatively, when the product is dominated by the SAR value, the cell sample which is associated with the higher signal activity value, is effectively the HSAS. Here, a particular cell sample may be both the LJDS and the HSAS, only the LJDS, or only the HSAS. One or another version of such possibilities occur for most prior art particular gene comparisons. For simplification in this discussion, a cell sample will be described as a LJDS or HSAS, or a HJDS or LSAS.

The first required condition for the certain occurrence of an (assay RASR)≠(ACR), related false negative result for a particular gene comparison, specifies that the particular gene must be actively expressed in each compared cell sample. As discussed earlier, in real life prior art gene comparisons, this condition is almost always met for thousands of genes in each compared cell sample. This is particularly true for mammalian cell sample gene activity comparisons where over 10,000 different genes are actively expressed in a typical mammalian cell sample comparison, and over half of these different genes are expressed in both compared cell samples as low mRNA abundance mRNA transcripts. In addition, the abundance of the commonly expressed low abundance mRNA transcripts, is similar but not necessarily identical, in each different cell sample. This large overlap between the low abundance mRNA populations of different related mammalian and other cell types, is common for mammalian and other eukaryote and prokaryote cell types, and their neoplastic offshoots. All this indicates that, in real life prior art microarray mammalian gene activity comparisons, the first requirement is met for the mRNA transcripts of as many as 5,000 different active genes.

The second requirement that, as a result of the assay JDQR and/or SAR value the (assay RASR)≠(ACR), is also met for almost all prior art microarray and non-microarray gene comparison results. Prior art generally believes that the assay RASR value for a particular gene comparison must be normalized, and that the NASR=ACR. Prior art considered assay variable NFs are utilized for this normalization process. All of these considered assay variable NFs, can affect the assay value for the JDQR, while the TSAR can affect the assay value for the SAR. In addition, the prior art unconsidered assay variable NFs, also affect the assay JDQR and SAR values.

The third requirement specifies that, the particular gene's mRNA abundance in the UDS or HSAS, must be equal to or less than, the gene's mRNA abundance in the HJDS or LSAS. As mentioned above, each cell sample in a mammalian cell sample comparison contains about 12,000-15,000 active genes, and about 10,000 or so of these active genes are low mRNA abundance level genes, which have an abundance level of 1-5 mRNA copies per cell. Over half the 10,000 or so low abundance mRNA genes are active in both compared mammalian cell samples, while the rest are detected as being active in only one cell sample. For simplicity it will here be assumed that for a mammalian cell comparison, about 5,000 low abundance 1-5 mRNA copy per cell genes are detected as being active in both compared cell samples, and about 5,000 low abundance 1-5 mRNA copy per cell genes are detected as being inactive in one cell sample and active in the other. Thus, for a mammalian cell sample gene comparison: 5,000 or so different low abundance 1-5 mRNA copy per cell genes are active in both cell samples, and an active gene in one cell sample has a mRNA abundance level which is equal to or similar to the abundance level of the same gene in the compared cell sample; 5,000 or so different low abundance 1-5 mRNA copy per cell genes are detected as active in one cell sample and not the other, and each detected active gene in one cell sample has low a mRNA abundance level which is similar to the mRNA abundance level of the same gene in the other compared cell sample. Prior art commonly practices that, for those genes which are active in both cell samples, and differentially expressed, about half are downregulated in one cell sample, and upregulated in the other cell sample. Thus, for a particular differentially expressed gene in a gene comparison assay, the probability of the downregulated gene being associated with the UDS or HSAS is about 0.5. In addition, the probability of the upregulated gene being associated with the HJDS or LSAS, is about 0.5. Therefore, about half of differentially expressed particular prior art genes meet this third requirement for both the LJDS or HSAS.

Prior art also commonly practices that for a typical cell sample gene comparison assay, the great majority of those genes which are active in both cell samples, are unregulated. Unregulated indicates that, a particular active gene has the same gene mRNA abundance level in each compared cell sample. In this event, for eukaryotic and prokaryotic cell sample gene comparisons, prior art believes that the majority of active in both cell samples genes, are unregulated low mRNA abundance genes. For mammalian cell sample gene comparisons, as many as 5,000 active in both cell samples genes, are unregulated, low mRNA abundance genes. Therefore, for many prior art eukaryotic and prokaryotic gene comparisons, the particular active gene's mRNA abundance in the LJDS or HSAS, is equal to the same gene's mRNA abundance in the HJDS or LSAS, and the third requirement is met. For prior art mammalian gene comparisons, this third requirement is met for 5,000 or so different particular low mRNA abundance level genes which are active in both cell samples.

A typical prior art microarray cell sample comparison detects as active a large number of low mRNA abundance level genes in each cell sample, and does not detect as active the same genes in the other cell sample. For a high density mammalian microarray thousands of low mRNA abundance level genes may be detected as being active in only one cell sample of the comparison. While the nature of these active in one cell sample low mRNA abundance genes is not known, many of them could meet this third requirement.

The fourth requirement specifies that a detectable amount of a particular gene mRNA LPN from the LJDS or HSAS, must be present in the assay. Put differently, the particular gene's mRNA abundance level in the UDS or HSAS must be detectable in the assay. The gene's LJDS or HSAS mRNA abundance level which is just detectable in the assay determines the gene's HJDS or LSAS mRNA abundance level around which the NS related false negatives can occur in the assay. For a particular gene comparison, the range of HJDS or LSAS mRNA abundance levels over which an NS related false negative can occur in an assay, is determined by the gene's LJDS or HSAS just detectable mRNA abundance level, and the magnitude of the deviation of the gene's assay RASR from the ACR. The higher the UDS or HSAS just detectable mRNA abundance level in the assay, the higher the gene's HJDS or LSAS mRNA abundance level must be in order for these NS related false negatives to occur. The greater the deviation of the gene's assay JDQR and/or SAR from one, and the greater the magnitude of the deviation of the gene's assay RASR from the ACR, the greater the HJDS or LSAS gene mRNA abundance level range, over which an NS related false negative can occur for the gene.

As discussed above, prior art believes that for a cell sample gene comparison, for those genes which are detected as being active in both cell samples and are differentially expressed, about half are downregulated in one cell sample and upregulated in the other cell sample. Consequently, about half the downregulated, differentially expressed, active in both cell samples genes, in a mammalian or other cell sample gene comparison assay, are associated with the UDS or HSAS and are detectable in the assay. Thus, about one quarter of the differentially expressed genes in a mammalian or other gene comparison meet the fourth requirement, if prior art beliefs and practices are correct.

As discussed above, prior art believes and practices that for a cell sample gene comparison, the great majority of the detected active in both cell sample genes, are unregulated genes. In this context, about 4,000-5,000 different active in both cell sample low mRNA abundance level genes, would be unregulated in a mammalian cell sample gene comparison. Each one of these unregulated genes is active in the UDS or HSAS. Under the proper microarray assay conditions each LJDS or HSAS unregulated low mRNA abundance level gene can be detected. Thus, in both prior art eukaryotic and prokaryotic cell sample gene comparisons, a large number of genes are unregulated, and can meet this fourth requirement.

As discussed, a typical prior art microarray cell sample gene comparison detects as active, a large number of low mRNA abundance level genes in each cell sample, and does not detect as active the same genes in the other cell sample. For a mammalian high density microarray, hundreds to thousands of low abundance level genes may be detected as being active in just one cell sample. Thus, the LJDS or HSAS in such an assay would be associated with hundreds to thousands of low mRNA abundance level 1-5 mRNA copy per cell genes, which are detectable as active only in the UDS or HSAS. Many of these genes could meet this fourth requirement.

The earlier discussed fifth requirement for the certain occurrence of SCR≠1 related false negative results in an assay, is essentially identical to this fourth requirement. The earlier discussion on the fifth requirement is also directly applicable here.

Requirements 1-4 for the certain occurrence of an (assay RASR)≠(ACR) related false negative result and RDM, appear to be met for a large number of individual genes in prior art microarray and non-microarray gene comparisons. The discussion of the real life relevance of the fifth requirement will assume that requirements 1-4 have been met.

The fifth requirement specifies the following. The magnitude of the gene's assay JDQR and/or SAR deviation from one, and the resulting deviation of the gene's assay RASR from the ACR, must be great enough so that the gene's HJDS or LSAS mRNA abundance level is not detectable in the assay. This must occur even though, the gene's UDS or HSAS mRNA abundance level is detectable in the same assay, and has a mRNA abundance level which is equal to or less than the gene's HJDS or LSAS mRNA abundance level. The larger the assay JDQR and/or SAR deviation from one, the greater the magnitude of the deviation of the assay RASR from the ACR. The further a gene's assay RASR deviates from the ACR, the higher the gene's HJDS or LSAS mRNA abundance can be, and still be undetectable in the assay, and the greater the difference can be in the assay between the detectable UDS or HSAS gene mRNA abundance, and the undetectable HJDS or LSAS gene mRNA abundance, and still have the occurrence of an NS related false negative result for the gene in the HJDS or LSAS. As an example, if the deviation of a gene's assay RASR value from the ACR is twenty fold, an HJDS or LSAS mRNA abundance level of 99 mRNA copies per cell for the gene will be undetectable in the assay, even though the UDS or HSAS mRNA abundance level for the same gene in the same assay is 5 mRNA copies per cell, and is detectable in the assay. Here, the HJDS or LSAS mRNA abundance level range over which an NS related false negative result for the gene can occur, is 5-99 mRNA copies per cell. If in a gene comparison assay, the HJDS or LSAS mRNA abundance level for the gene is less than 5 copies per cell or 100 or more copies per cell, an NS related false negative cannot occur for the gene in the HJDS or LSAS. Whether the HJDS or LSAS gene's mRNA abundance level coincides with the 5-99 copies per cell abundance level range over which an NS related false negative result for the gene will occur, depends on biological factors.

Given that requirements 1-4 appear to be met for a large number of prior art prokaryotic and eukaryotic particular gene comparisons, the real life relevance of the fifth requirement hinges upon: (a) Whether the magnitude of the deviation of the assay RASR values from the ACR values which generally occurs in prior art gene comparisons is enough to cause the occurrence of NS related false negative results and RDMs; (b) The number, in a typical prior art gene comparison assay, of different, active in both cell samples genes, each of which has an HJDS or LSAS cell mRNA abundance level, which overlaps the mRNA abundance level range over which an NS related false negative result can occur for the gene in the HJDS or LSAS.

As discussed earlier, prior art considered and unconsidered NFs can cause a gene's assay JDQR and/or SAR to deviate from one, and can therefore cause an (assay RASR)≠(ACR) related false negative result for the gene. For the purposes of this discussion, it has been assumed that the prior art gene comparison assay SCR=1, even though it is often not equal to one. Prior art generally believes that a particular gene's prior art produced (assay RASR)≠(ACR), and that the gene's assay RASR must be converted by a prior art normalization process, to an assay NASR value. Prior art believes that this prior art produced (assay NASR)=(ACR). It is estimated that prior art particular gene comparison assay measured RASR values commonly deviate from the ACR by 2 to 4 fold or more. Prior art often attributes such deviation to the deviation of the assay global NF TSAR value from one. In this event, the assay RASR values for all of the genes compared in the assay deviate from the ACR by 2 to 4 fold, absent other influences. For many, if not most, published prior art microarray gene comparison assay results, the quantitative magnitude of the normalization correction factor which converts the assay RASR to the assay NASR, is not reported. Further, the prior art produced assay NASR is not corrected for prior art unconsidered NFs, and therefore may be incompletely normalized. Consequently, it is reasonable to believe that many prior art assay RASR values may deviate from ACR more than 2 to 4 fold. It is clear from earlier discussions on the effect of different prior art considered and unconsidered NF values on the relationship (assay RASR)=(ACR), that under certain plausible prior art assay conditions which are not uncommon, prior art produced assay RASR values could deviate from the ACR by a factor of 5-10 fold, or more. It is important to recognize that the prior art normalization process cannot, and does not, correct for the presence of prior art considered NS related false negative results and RDMs which occur in a gene comparison assay. A normalization process which perfectly corrects assay RASR results for all pertinent assay variables, also does not and cannot, correct for the presence of NS related false negative results. The best that can be done is to either: design the microarray assay to prevent or minimize the occurrence of such false negative results; or to determine the assay mRNA abundance levels at which the false negatives can occur, and take that into consideration when interpreting the assay positive, and negative results.

The above discussion indicates that, it is common for prior art produced gene comparison assay RASR values to deviate from the ACR by 2 to 4 fold, and that the deviation may be much greater for many prior art particular gene comparisons. It is clear that such 2 to 4 fold deviations are large enough to cause NS related false negative results and RDMs if all five requirements are met. Table 38A & B (which together represent one table) illustrate this. The examples of Table 38A & B are presented in terms of the HJDS and LJDS. However, Table 38A & B also applies directly to HSAS and LSAS situations. These examples illustrate that NS related false negative results occur only when the HJDS gene A mRNA abundance level falls within the HJDS mRNA abundance level range over which these NS related false negatives can occur (see Table 38A & B i-iv and vi-viii).

TABLE 38A
Occurrence of NS Related False Negative Results in HJDS or LSAS

| | | | Gene's Just | | |
|---|---|---|---|---|---|
| | | | Detectable | Gene's | Gene's |
| | | | LJDS | LJDS | HJDS |
| | | | mRNA | mRNA | mRNA |
| | | | Abundance | Abundance | Abundance |
| Gene | | Cell Sample^{(c)} | Level in | Level for | Level for |
| Compared | | Compared | Assay (CPC) | Assay (CPC) | Assay (CPC) |
| (i) | | LJDS | 3 | 3 | 3 |
| | A | HJDS | | | |
| (ii) | | LJDS | 300 | 300 | 300 |
| | A | HJDS | | | |
| (iii) | | LJDS | 3 | 3 | 4.49 |
| | A | HJDS | | | |
| (iv) | | LJDS | 300 | 300 | 400 |
| | A | HJDS | | | |
| (v) | | LJDS | 3 | 3 | 9 |
| | A | HJDS | | | |
| (vi) | | LJDS | 3 | 3 | 8.9 |
| | A | HJDS | | | |
| (vii) | | LJDS | 10 | 20^{(b)} | 29 |
| | A | HJDS | | | |
| (viii) | | LJDS | 5 | 5^{(b)} | 24 |
| | A | HJDS | | | |

(a) CPC = mRNA copies per cell.
^{(b)}HJDS mRNA abundance level range over which NS related false negatives can occur.
For (viii) the range is 5 to <25 gene A mRNA copies per cell.
For (vii) the range is 20 to <30.
^{(c)}Assay SCR = 1, for all examples.

TABLE 38B
Occurrence of NS Related False Negative Results in HJDS or LSAS

| | | | | Gene's Just | | |
|---|---|---|---|---|---|---|
| | | | | Detectable | | |
| | | | Gene's | HJDS | | Occurrence of |
| | | | Assay RASR | mRNA | Detectability | NS Related |
| | | | Deviation | Abundance | of Gene | False Negative |
| Gene | | Cell Sample^{(c)} | From | Level in | Activity in | Result for |
| Compared | | Compared | AHCR | Assay (CPC) | Assay | Gene in HJDS |
| (i) | | LJDS | 1.5 | 4.5 | Yes | Yes |
| | A | HJDS | | | No | |
| (ii) | | LJDS | 1.5 | 450 | Yes | Yes |
| | A | HJDS | | | No | |
| (iii) | | LJDS | 1.5 | 4.5 | Yes | Yes |
| | A | HJDS | | | No | |
| (iv) | | LJDS | 1.5 | 450 | Yes | Yes |
| | A | HJDS | | | No | |
| (v) | | LJDS | 3 | 9 | Yes | No |
| | A | HJDS | | | Yes | |
| (vi) | | LJDS | 3 | 9 | Yes | Yes |
| | A | HJDS | | | No | |
| (vii) | | LJDS | 3 | 30^{(b)} | Yes | Yes |
| | A | HJDS | | | No | |
| (viii) | | LJDS | 5 | 25^{(b)} | Yes | Yes |
| | A | HJDS | | | No | |

(a) CPC = mRNA copies per cell.
^{(b)}HJDS mRNA abundance level range over which NS related false negatives can occur.
For (viii) the range is 5 to <25 gene A mRNA copies per cell.
For (vii) the range is 20 to <30.
^{(c)}Assay SCR = 1, for all examples.

The incidence of occurrence of these NS related false negative results in typical prior art microarray and non-microarray gene comparisons, depends upon the number of HJDS or LSAS active in both cell samples genes present in such a gene comparison assay, which have mRNA abundance levels which coincide with the HJDS or LSAS mRNA abundance level over which such false negative results can occur. The magnitude of this gene number in prior art gene comparisons, is discussed below.

As illustrated in Table 38A & B, NS related false negatives and RDMs can occur at high or low abundance levels. For a typical prior art gene comparison, the number of active in both cell sample genes which have a high cell mRNA abundance level, is relatively small. In mammals, the medium and high abundance genes comprise roughly 5-10 percent of the total number of expressed genes. The incidence of occurrence of NS related false negatives for these medium and high abundance genes will be relatively small due to the small numbers involved. In contrast, it has been estimated that about 0.85 of the expressed genes in mammalian cell samples, or roughly 9,000 genes, have a mRNA abundance level of 1-5 mRNA copies per cell. As discussed earlier, for a typical mammalian cell sample comparison, about 5,000 of the same 1-5 copy per cell genes, are actively expressed in both cell samples. In addition, the cell mRNA abundance of a particular active 1-5 copy per cell low abundance gene in one cell sample, is similar to or equal to, the cell mRNA abundance level of the same 1-5 copy per cell low abundance gene, present in the other cell sample. Prior art believes that generally, only a small number of these active in both cell sample 1-5 copy per cell low mRNA abundance genes, are differentially expressed. For those active in each cell sample 1-5 copy per cell low mRNA abundance genes which are differentially expressed, the maximum T-DGER=5, and it is likely that most of these genes will differ in expression by 2-3 fold. Prior art also commonly practices that for a typical mammalian cell sample gene comparison, the great majority of those 1-5 copy per cell, low mRNA abundance genes which are active in both cell samples, or about 4,000-5,000 genes, are unregulated, and have a T-DGER=1. For a typical prior art mammalian cell sample gene comparison, each of these 4,000-5,000 unregulated 1-5 copy per cell low abundance genes, meets requirements 1-4. The potential incidence of occurrence of NS related false negative results and RDMs in a prior art mammalian cell sample comparison, is analyzed below.

As discussed above, prior art generally believes that for all microarray eukaryotic and prokaryotic gene comparisons, the (assay RASR)≠(ACR). Further, it is common for prior art produced assay RASR values to deviate from the ACR by at least 2 to 4 fold. It is not uncommon for a prior art microarray mammalian cell sample gene comparison assay, to have a UDS or HSAS just detectable cell mRNA abundance level of 3-5 mRNA copies per cell. Here, for simplicity, the following will be assumed. (a) The LJDS or HSAS just detectable mRNA abundance level is 3 mRNA copies per cell, for each of the different 5,000 or so unregulated 1-5 copy per cell low cell mRNA abundance genes. (b) The magnitude of the deviation of each genes assay RASR from the ACR, is 1.5 or 3 fold. This situation is illustrated in Table 38A & B. Table 38A & B (i) (iii) indicates that in this situation, when a gene's deviation is 1.5 fold, then the HJDS or LSAS mRNA abundance level range over which a false negative will occur for a gene in the HJDS or LSAS, is 3 to almost 4.5 copies per cell. In this situation, the UDS or HSAS just detectable mRNA abundance level of 3 copies per cell, closely coincides with the 3-4.5 copy per cell HJDS or LSAS cell mRNA abundance level, over which an NF related false negative can occur for the HJDS or LSAS 1-5 copy per cell low mRNA abundance level genes. Here, of the 5,000 or so 1-5 copy per cell low mRNA abundance HJDS or LSAS genes, the ones which have a HJDS or LSAS mRNA abundance level of 3 to about 4.9 mRNA copies per cell, will not be detected in the assay, and therefore will be associated with NS related false negative results and RDMs. This HJDS or LSAS mRNA abundance level range of about 1.5 fold, represents about one third of the 1-5 copy per cell low cell mRNA abundance level range, which comprises 5,000 or so different mammalian active genes. It is not known how many HJDS or UDS genes are actually present, in this 3-4.9 copy per cell region of the low cell mRNA abundance level genes. However, if it is assumed that the genes are evenly distributed over the 1-5 copy per cell range, the number of NS related false negative results which will occur in this typical mammalian cell sample gene comparison assay, is roughly 1,500. In the above assay situation, if the assay RASR deviates from the ACR by 3 fold, the HJDS or LSAS mRNA abundance level over which an NS related false negative result can occur, ranges from 3 to almost 9 copies per cell (see Table 38A & B v, vi). In this event, nearly half of the 5,000 or so HJDS or LSAS low mRNA abundance genes can be associated with NS related false negative results.

As discussed above, for a typical prior art microarray cell sample gene comparison, the LJDS or HSAS is associated with a large number of low mRNA abundance level 1-5 mRNA copy per cell genes, which are detectable as active only in the LJDS or HSAS. Each of these LJDS or HSAS active genes is not detectable in one of the compared cell samples. In a high density microarray mammalian cell comparison, the number of genes in each of the said, active only in the LJDS or HSAS, and inactive in the HJDS or LSAS, categories can be thousands. For a cell sample gene comparison, many of the same inactive undetected genes in the HJDS or LSAS, which are active in the LJDS or HSAS, may in fact be active, and meet the fifth requirement.

The above discussed considerations indicate that the fifth requirement appears to be met for a large fraction of HJDS or LSAS low mRNA abundance level genes, under certain, not uncommon prior art assay conditions used for mammalian and other cell sample gene comparisons. The above discussion has focused on whether the fifth requirement was met for a significant number of prior art mammalian HJDS or LSAS low mRNA abundance level genes. However, the discussion also applies to differentially expressed HJDS or LSAS genes at any mRNA abundance level, as well as to HJDS or LSAS unregulated genes at any abundance level. The discussion and conclusions also apply to many prior art non-mammalian eukaryotic and prokaryotic gene comparison HJDS or LSAS high, medium, and low mRNA abundance level genes.

It appears likely that a significant number of prior art particular low mRNA abundance level gene comparisons meet the 5 requirements and are associated with NS related false negative results and RDMs. Overall, it appears that the prior art occurrence of NS related false negative results and RDMs, is not uncommon. Interpretation of NS Related False Negative Results Associated with (Assay RASR)≠(ACR).

These NS related false negative results cannot occur for a particular gene comparison when, the pertinent assay NF values are equal to one, or enough mRNA or equivalents from both cell samples is added to the assay ensures the detection of the least abundant mRNA in each cell sample being compared. Neither of these conditions is often met in mammalian gene activity comparisons. For prior art prokaryote and eukaryote gene expression comparisons, the first condition is not met. Prior art generally believes that all gene comparison assay RASR results need to be corrected or normalized in order to obtain biologically relevant or meaningful gene comparison assay results. The second condition, while not often met, is met much more often for prokaryotes and simple eukaryotes, than for mammals. The consequence of not meeting one or the other of these conditions, is discussed below. For this discussion, it will again be assumed that the SCR=1, and that any NF related false negative result will be an NS related false negative result.

In reality, a typical mammalian gene comparison assay meets neither of the conditions. In such a comparison, when a positive result which is associated with a relatively low assay signal value is obtained for a gene in the LJDS or HSAS, and a negative result is obtained for the same gene in the HJDS or LSAS, the interpretation of the HJDS or LSAS negative result is uncertain. The HJDS or LSAS negative result, could be caused by one of three different situations which might exist in the HJDS or LSAS. In the first situation, the gene is inactive in the HJDS or LSAS, and therefore the negative result is a true negative result. Here, an interpretation that, relative to the UDS or HSAS, the HJDS or LSAS gene is downregulated, would be correct. In the second situation, the HJDS or LSAS gene is active, but not active enough to be detected, even if the assay JDQR or SAR is equal to one. This situation produces a false negative result which is not related to the assay NF values. Here, any interpretation that, relative to the LJDS or HSAS gene, the HJDS or LSAS gene is downregulated, would be correct. In the third situation, on a mRNA copy per cell basis, the activity of the HJDS or LSAS gene is equal to or greater than, the activity of the same gene in the LJDS or HSAS, and because the (assay RASR)≠(ACR), an NS related false negative result is produced for the HJDS or LSAS gene. In this third case, an interpretation that, relative to the UDS or HSAS gene, the HJDS or LSAS gene is downregulated, is incorrect.

For a particular prior art gene comparison where a negative result is obtained for a gene in one cell sample, and a positive result associated with a relatively low assay signal is obtained for the same gene in a different cell sample, the interpretation of the gene's activity in the negative cell sample is uncertain. In reality, the negative cell sample gene could be active or inactive. In addition, the interpretation of the direction of gene regulation differences between the active gene cell sample and the inactive gene cell sample, is also uncertain. In reality, relative to the gene in the positive cell sample, the gene in the negative cell sample could be unregulated, upregulated, or downregulated. Absent some knowledge of the gene comparison assay JDQR or SAR values, and the gene's HJDS or LSAS mRNA abundance level range over which an NS related false negative can occur in the assay, the interpretation for such a prior art negative result is uncertain. Prior art practice for microarray and non-microarray gene comparisons does not determine a gene's assay PNFP, or the assay mRNA abundance level range over which such NS related false negatives can occur. In addition, prior art gene comparison assays rarely involve enough cell sample mRNA or equivalents in the assay, to ensure the detection of the least abundant mRNA in each cell sample being compared. Thus, for such a prior art situation where a positive gene activity result associated with a relatively low assay signal is obtained for a gene in one cell sample, and a negative gene activity result is obtained for the same gene in a different cell sample, the interpretation of the negative result is uncertain for mammalian, as well as other eukaryote and prokaryote prior art gene comparisons. Note that if the deviation of a particular gene RASR value from the particular gene ACR value is large enough, the positive assay result associated with an NS related false negative can be quite large.

Interpretation of Assay Variable NF Related False Negative Results Associated with Prior Art Gene Expression Activity Comparison Assays.

A particular gene comparison associated with an NF related false negative result has the following characteristics. One. The particular gene is detected as being active in one cell sample, and the assay signal associated with this active gene is generally relatively low. Two. The particular gene is detected as being inactive in the other compared cell sample. Three. The gene's mRNA copy per cell abundance level in the inactive or negative cell sample, is equal to or greater than the same gene's mRNA copy per cell abundance level in the active or positive cell sample. Four. As a result of three, the gene's mRNA copy per cell abundance level in the active or positive cell sample, is equal to or less than the gene's mRNA copy per cell abundance level in the inactive or negative cell sample. Five. Such an NF related false negative result for the gene will occur in the negative cell sample at an mRNA per cell abundance level which is equal to or greater than the just detectable mRNA copy per cell abundance level, for the gene in the active or positive cell sample. Sixth. The mRNA copy per cell abundance level range in the negative cell sample over which an NF related false negative can occur in the negative cell sample, is determined by the particular gene comparison's pertinent NF assay values.

Two different types of assay variable related false negative results and their associated RDMs, have been discussed. One of these is the EA Rule, or SCR, related false negative results. An EA Rule or SCR related false negative result is caused by the almost universal practice of the EA Rule for microarray and non-microarray gene activity comparison analysis. The second of these is the non-SCR, or NS, related false negative results. An NS related false negative result is associated with one or more of the prior art considered or non-SCR prior art unconsidered assay variable NFs. The prior art considered NS assay variable NFs include but are not limited to, the ARR, TSAR, C-HKR, spatial, print tip, print plate, intensity, scale, AE•AER. The prior art unconsidered assay variable NFs include but are not limited to SCR, PAFR, MLDR, PL-HKR, PS-HKR, PSAR, PSSR, LLSR, SBNR, and SSAR assay variable NFs. EA Rule related false negative values occur when the assay SCR≠1. NS related false negative values occur when, for a particular gene comparison, the (assay RASR)≠(ACR). This occurs when the assay value for one or more of the NS NFs deviates significantly from one.

SCR related and NS related false negative results and the associated RDMs, cannot occur for a particular gene comparison when one of the following conditions is met. (i) Enough cell sample mRNA LPN is added to the assay hybridization solution to ensure the detection of the least abundant mRNA LPN in each compared cell sample. (ii) An equal number of cells from each cell sample is compared, i.e., the assay SCR=1, and the assay value for each of the assay pertinent NS assay variable NFs, is equal to one. None of the above conditions is often met for prior art gene expression activity comparisons. It is not clear whether the first condition has ever been met, even for a prokaryotic gene expression analysis. A typical mammalian gene comparison assay meets neither of the two conditions.

An earlier discussion on the incidence of EA Rule related false negative results in real life indicated that, EA Rule or SCR related false negative results are not uncommon in prior art microarray and non-microarray prokaryotic and eukaryotic gene activity comparison analyzes, and the number of such false negative associated with prior art mammalian gene comparisons may be very high. Similarly, an earlier discussion on the incidence of NS related false negative results in real life indicated that NS related false negative results are not uncommon in prior art prokaryotic and eukaryotic gene comparison analyzes, and the number of such false negatives associated with prior art mammalian gene comparisons may be very high. In one prior art gene comparison assay the EA Rule related and NS related false negative results can occur together. Here, depending on the assay values for the SCR and the NS assay variable NFs, the combined incidence of SCR and NF related false negative results in a prior art gene comparison may be significantly higher, or lower, than the estimated incidence of either the SCR related, or NS related false negative results.

Prior art does not determine, or take into consideration during the normalization of particular gene comparison assay RASR results, the assay SCR value, or the assay values for the prior art unconsidered assay variable NFs. Prior art does determine, or take into consideration during the normalization process, the assay values for the prior art considered NFs. However, even if the assay values for all pertinent considered and unconsidered NFs were known, a normalization process which perfectly normalizes the gene comparison results for these assay variables, cannot correct for the presence of the SCR related or NS related false negative results and RDMs which occur in the assay. Overall, the best that can be with regard to minimizing the occurrence of such false negatives in an assay, or minimizing the effect of their occurrence on the interpretation of the assay results, is to either design the microarray assay to prevent or minimize the occurrence of such false negative results and/or determine the assay mRNA abundance levels at which the false negatives can occur, and take that information into consideration when interpreting the assay positive and negative gene activity results which occur at all mRNA abundance levels. Prior art gene comparison practice does neither of these.

For a particular prior art gene comparison, where a positive result which is associated with a relatively low assay signal is obtained for a gene for one cell sample, and a negative result is obtained for the same gene in the other compared cell sample, the interpretation of the gene's activity in the negative cell sample is uncertain. In reality, the interpretation of whether the negative cell sample gene is active or not, is uncertain, as is the interpretation of the direction of the gene regulation difference between the active gene in one cell sample, and the negative or measured inactive gene in the compared cell sample. The negative gene in the one cell sample may, in reality, be actively expressed in that cell sample. Further, relative to the measured active gene in the positive cell sample, in reality the measured inactive gene in the negative cell sample could be unregulated, upregulated, or downregulated. Absent some knowledge of the particular gene comparison's assay values for SCR and the NS assay variable NFs, and the negative cell sample's gene mRNA abundance level over which the SCR and/or NS related false negatives can occur in the assay, the interpretation for such a prior art negative result is uncertain. Prior art practice for microarray and non-microarray particular gene comparisons does not determine whether or not prior art considered or unconsidered assay variable NF related false negatives can occur in a microarray or non-microarray gene comparison assay. Nor does prior art practice determine the gene mRNA abundance range over which such NF related false negatives can occur in the assay. In addition, prior art gene comparison assays rarely, if ever, involve enough cell sample mRNA LPN in the assay to ensure the detection of the least abundant mRNA in each cell sample being compared.

The consequence of all this is that for the many prior art gene comparison instances where a relatively low assay signal positive result is obtained for a particular gene in one cell sample, and a negative result is obtained for the same gene in a compared cell sample, the interpretation of the negative result is uncertain for mammalian, as well as other eukaryote and prokaryote prior art gene comparisons. This means that such particular said prior art negative results are essentially uninterpretable.

A typical prior art microarray cell sample gene expression analysis is associated with a large number of different particular gene comparisons where a gene in one cell sample is measured as positive or active and is associated with a relatively low assay signal, while in the other compared cell sample the same gene is measured to be negative or inactive. Hundreds to thousands of such prior art gene comparison results occur in a typical high density microarray mammalian gene comparison assay. The great majority of such mammalian particular gene comparison results involve particular low mRNA copy per cell abundance level genes. In a typical prokaryotic or non-mammalian eukaryotic gene comparison assay, large numbers of such gene comparison results also occur.

As discussed earlier, an important and powerful extension of microarray and non-microarray gene expression analysis involves data mining and systems biology analyzes. As an example, prior art endeavors to identify which particular genes are active or expressed, and which particular genes are inactive or not expressed in a cell sample or cell samples exposed to some chemical or other stimulus. One basic data mining method is to group together the genes which are active in the cell samples, and genes which are inactive in the cell samples, and chart which genes become active in response to the stimulus and which become inactive. Inferences about the effect of the stimulus on gene regulation patterns are then often made from such prior art produced particular gene comparison active and inactive results. For a typical microarray gene comparison assay, a large number of particular gene comparisons associated with low mRNA copy per cell abundance level genes, result in a gene in one cell sample being measured as positive, and the same gene in another cell sample being measured inactive or negative. As discussed, the actual gene activity state which exists in the cell sample for each of such particular measured negative genes, and the actual regulation direction relationship which exists between the measured negative gene in one cell sample, and the same measured positive gene in another cell sample, cannot be known to be correct when two cell samples are compared in an assay. The interpretation of such measured inactive genes is therefore uncertain. Adding more cell samples to the gene comparison assay compounds the interpretation of such measured negative results further. Because of these uncertainties in the interpretation of such measured negative gene results, the interpretation of prior art data mining and systems biology analysis results which rely on the correctness of the prior art interpretation of these negative results, are uncertain.

The above discussion applies directly to DGSS, DGDS, and SGDS gene comparisons of all kinds.

I. Validity of Prior Art Normalization of Corroborative Non-Microarray Gene Expression Comparison Assay Results.

As discussed earlier, prior art microarray practice uses non-microarray gene comparison methods in order to validate or corroborate microarray gene comparison results (133, 198, 199). Such non-microarray methods include the methods of northern blot, dot blot, nuclease protection, and RT-PCR. Prior art believes and practices that both prior art normalized microarray and non-microarray particular gene comparison assay NASR values are biologically correct and can be validly intercompared.

Prior art believes and practices that it is necessary for each different type of non-microarray assay method to control for the amounts of RNA or equivalents compared in the assay. To accomplish this, many prior art non-microarray and corroborative methods utilize housekeeping genes, which are believed to be unregulated, as internal controls. The assay results from the housekeeping genes are then used to normalize the assay particular gene comparison results for differences in the amounts of RNA, or equivalents, compared in the assay, as well as other assay variables. For northern blot, such other assay variables include, but are not limited to, differences in, RNA purity, RNA integrity, RNA immobilization efficiency, hybridization availability of RNA, and quantitation of hybridization, for the compared RNAs. For nuclease protection, such other assay variables include, but are not limited to, differences in, RNA purity, RNA integrity, hybridization conditions, and quantitating the hybridization, for the compared RNAs. For RT-PCR, such other assay variables include, but are not limited to, differences in, RNA purity, RNA integrity, efficiency of cDNA synthesis, integrity of cDNA, purity of cDNA, amplification efficiency of cDNA and resulting amplicons, and quantitating the resulting cDNA amplicons, for the RT-PCR assay. Prior art occasionally utilizes added exogenous polynucleotide molecules to each compared RNA in order to control for and normalize for certain of these assay variables. Such variables include, but are not limited to, differences in, RNA purity, hybridization availability of RNA, quantitation of hybridization, hybridization conditions, certain limited aspects of cDNA synthesis efficiency, and certain limited aspects of cDNA and amplicon amplification efficiency.

All of the prior art non-microarray or corroborative methods have relied heavily on the putative housekeeping genes in order to control and normalize for the amount of RNA or equivalents compared, as well as other assay variables. As discussed earlier, the prior art currently acknowledges that housekeeping genes with general utility have not been identified. However, a few prior art microarray and non-microarray gene comparison practitioners, believe and practice that unregulated housekeeping genes which are applicable to particular cell sample comparisons have been identified, and are valid for normalization purposes. Prior art identifies such limited use housekeeping genes using prior art microarray and non-microarray gene comparison methods. As discussed earlier, these prior art microarray and non-microarray gene comparison methods do not take into consideration the prior art unconsidered global and non-global assay variables discussed earlier. As a consequence many prior art microarray and non-microarray particular gene comparison assay NASR values are biologically incorrect, and the vast majority of the other prior art microarray and non-microarray particular gene comparison assay NASR values cannot be known to be correct or incorrect. Therefore, many of the identified limited use housekeeping genes are likely not to be true limited use housekeeping genes, and the others may, or may not be true limited use housekeeping genes. As discussed earlier, even if prior art identified, true general and limited use housekeeping genes were known, their utility and applicability for normalizing other particular gene comparisons in an assay is severely limited by the existence of prior art unconsidered non-global assay variables associated with prior art microarray and non-microarray assays.

The considerations discussed indicate the following. The prior art belief and practice that a prior art non-microarray or corroborative assay measured NASR value for a particular gene comparison is biologically correct, is erroneous for many particular gene comparison NASR values, and cannot be known to be correct or incorrect for many other particular gene comparison NASR values. This occurs because the prior art non-microarray assay result normalization practice does not take into consideration during the normalization process for non-microarray particular gene expression results, all of the pertinent considered and unconsidered assay variables. The result of this is many prior art non-microarray particular gene comparison results which can be known to be incompletely normalized, and therefore biologically incorrect, and many other prior art non-microarray results for which it cannot be known whether the results are incompletely normalized or not. Thus, the prior art normalization of non-microarray or corroborative assay particular gene comparison results can be known to be invalid for many such prior art results, and for others it cannot be known whether the prior art normalization of such gene comparison results is valid or not.

Validity of Prior Art Practice of Validating Microarray Results with Non-Microarray Gene Comparison Method Results.

Prior art belief and practice is that it is necessary to validate or corroborate microarray particular gene comparison assay NASR values, and that this is done using a non-microarray gene expression comparison method to independently determine the assay NASR for the particular gene comparison. In practice, these prior art non-microarray or corroborative results often appear to verify the prior art microarray results, and somewhat less often, they do not. The discussion in the previous section indicates that the prior art normalization of prior art non-microarray assay results is often not valid, and in many other instances, it cannot be known to be valid or invalid. Because of this, the prior art belief and practice that a prior art non-microarray or corroborative assay measured gene comparison NASR value can be validly compared to the same cell sample gene comparison NASR value obtained using a different non-microarray or corroborative method, or a microarray method, is not valid for many such comparisons, and cannot be known to be valid for many others. The effect of this situation on the interpretation of prior art non-microarray or corroborative assay results is discussed below.

When the microarray assay result is not verified by the non-microarray corroborative method result, in reality the prior art cannot know whether either assay NASR value is biologically correct or not. What is known is that the two methods disagree on the particular gene comparison value. In a situation where the microarray assay and the non-microarray assay NASR values agree, it cannot be known by the prior art whether either assay NASR value is biologically correct or not. It can only be known that the two results agree. This uncertainty in the interpretation of corroborative results occurs because the prior art microarray and non-microarray particular gene comparison results which are compared, cannot be known to be completely and validly normalized for all assay associated sources of experimental and biological bias. Prior art does not determine or consider for the prior art normalization process, the prior art unconsidered global and non-global assay variables which have been identified and discussed herein. Absent knowledge concerning the unconsidered assay variables associated with each particular microarray and non-microarray assay particular gene comparison result, the interpretation of the compared microarray and corroborative assay particular gene comparison results cannot be clarified.

Note that when the prior art assay NASR value for a particular gene comparison obtained for one type of non-microarray method, is compared to the prior art assay NASR value for the same particular gene comparison obtained with a different type of non-microarray method, the interpretation is uncertain. Absent further information, it cannot be known if either result is biologically correct or not. Note further that in none of the above situations does prior art determine or consider the information necessary to clarify the interpretation.

### III. DESCRIPTION OF EXEMPLARY APPLICATIONS AND PRACTICES OF THE PRESENT INVENTION

The invention comprises a novel method and means for obtaining microarray and non-microarray and clone counting assay gene expression analysis results, gene expression comparison results, and gene expression comparison data mining and systems biology analysis results, which are known to be improved relative to prior art obtained microarray and non-microarray and clone counting assay obtained gene expression analysis results, gene expression comparison analysis results, and gene expression comparison data mining and systems biology analysis results. Such improved results include assay results from SGDS, DGDS, and DGSS assay comparisons of viral, prokaryotic, eukaryotic, and standard RNA transcripts of all kinds. This includes all types of rRNA, tRNA, mRNA, siRNA, miRNA, snoRNA, antisense RNA, and other known and unknown RNA transcripts. The implementation of the method and means of the invention involves: (a) The identification, definition, and experimental measurement, of assay related global and non-global assay variables which have been previously unknown or unconsidered in the prior art normalization process; (b) The method of consideration of these said global and non-global assay variables for the normalization of microarray and non-microarray gene expression analysis results; (c) The use of microarray and non-microarray assay design to simplify the process of said normalization.

The methods and means of the invention can be readily incorporated into existing microarray and non-microarray gene expression analysis, and gene expression comparison analysis, and associated data mining and systems biology analysis methods as well as pharmaceutical and many other applications. One aspect of the incorporation of these methods and means requires the experimental determination of one or more experimentally derived results or items of information which are produced separately from the gene expression analysis results. These separately produced experimental results include, but are not limited to, one or more of the following. (a) A quantitative measure of the absolute or relative number of cells or cell equivalents analyzed in the assay. (b) A quantitative measure of the absolute or relative amount of total mRNA transcript molecules per cell for the analyzed cells or cell samples. (c) A quantitative measure of the absolute or relative amount of total RNA per cell for the analyzed cells or cell samples. (d) A quantitative or relative measure of the fraction of each particular mRNA type which is PA mRNA in the analyzed cells or cell samples. (e) A quantitative measure of the absolute or relative differences in the nucleotide length of the PG mRNA LPN molecule populations, which are analyzed or compared in the assay. (f) A quantitative measure of the absolute or relative nucleotide composition for the mRNA LPN molecule populations which are analyzed or compared in the assay. (g) A quantitative measure of differences in the nucleotide sequences in an assay of a particular genes compared mRNA LPN molecules. (h) A quantitative measure of the absolute or relative total nucleotide complexity (TNC) for a particular gene's mRNA LPN molecule populations which are analyzed or compared in an assay. (i) A quantitative measure of the absolute or relative TPN value for a particular genes mRNA LPN populations which are analyzed or compared in the assay. (j) A quantitative measure of the absolute or relative TSA values for the mRNA LPN preparations compared in the assays. (k) A quantitative measure of the absolute or relative PSA values for a particular genes mRNA LPN molecule population which is analyzed or compared in an assay. (1) A quantitative measure of the label density (LD) of each particular gene's compared or analyzed LPN molecule population. (m) A quantitative measure of the absolute or relative ECDP value for each gene present in the assay. The determination of each of these is described below. Also described are microarray and non-microarray, and clone counting assay methods, which simplify the improved normalization process. For these descriptions, SGDS comparisons of particular gene mRNA transcripts will be emphasized. However, these descriptions apply directly to SGDS, DGDS, and DGSS, comparisons of viral, prokaryotic, eukaryotic, and standard RNA transcripts of all kinds. This includes all types of rRNA, tRNA, mRNA, siRNA, miRNA, snoRNA, antisense RNA, and other known and unknown RNAs.

A. Determination and Normalization of Assay Variables

Determination of Absolute and Relative Number of Cells in a Sample.

There are a variety of established methods for measuring the number of cells present in a cell sample, and for measuring the number of cell equivalents present in a cell sample (16, 200, 201). In many cases, the number of cells present can be determined by the direct counting of cells. There are a variety of prior art methods to accomplish this. Such methods include, but are not limited to, counting individual cells with a hemacytometer or an automatic cell counting device, such as a Coulter counter or a flow cytometer.

Alternatively, the number of cells present in a cell sample may be determined by quantitatively measuring some physical or chemical property or activity of the cells of interest which correlates accurately with cell number. As an example, the quantitative value for the DNA content per haploid cell is known for many prokaryotic organisms (10, 11), and for a particular type of prokaryote all haploid cells have the same DNA content. A similar situation exists for eukaryotes where all diploid cells of a particular type of eukaryote have the same DNA content (15). For example, all of the different types of diploid cells which make up a human have the same DNA content per cell, and a diploid cell in one human has the same DNA content per cell as a diploid cell in any other human. In this situation, the relative number of cells for two different diploid human cell samples can be determined by directly comparing the total DNA contents of each cell sample. The ratio of these two DNA contents per cell sample is then a measure of the relative number of cells in each cell sample, or the sample cell ratio SCR for the two cell samples. Thus, it is not necessary to determine the absolute number of cells present in each of two diploid cell samples, or each of two diploid cell samples, from the same organism type or very similar organism types, in order to obtain a measure of the quantitative SCR value for a comparison. In this instance, if the human diploid DNA content per cell is known, the absolute number of cells in each different cell sample can be determined by dividing the total DNA content of the cell sample by the diploid DNA content per cell. It should be noted that in reality it is not uncommon for the average ploidy of a prokaryotic or eukarotic, including mammalian, cell to be greater than the strict diploid state. How much greater depends on the differentiation, growth, and metabolic states, of the cells of each sample. Note that most culture cells are associated with aneuploidy. For certain continuous mammalian cell lines, the cells are approximately tetraploid in nature. Further, a single mammalian cell line type, such as HeLa cells, can have significantly different degrees of aneuploidy in different laboratories. It cannot be assumed that the ploidy of different cultured cells from one organism type or from one organism, such as a human, are the same. It is believed however, that the diploid DNA contents of essentially all cell varieties in one organism type, such as human, are the same. However, even in these cells the DNA content per cell is greater than the diploid DNA content per cell at particular stages of the cell cycle. This occurs for both prokaryotes and eukaryotes. In addition, because of the pattern of bacteria DNA replication, in a rapidly growing bacterial cell the copy per cell number for those bacterial genes which are located near the origin of chromosomal DNA replication, can vary up to four fold during the cell cycle. This could mean that the mRNA abundance values of these genes mRNAs vary four fold or so, over the cell cycle (10, 11). For a cell sample gene expression comparison of fast and slow growing cells, this could affect the magnitude of the measured expression differences, and should be taken into consideration in the interpretation of the assay results. It is likely that such a situation also exists in mammalian and other eukaryotic cells. It is preferable to determine both the number of cells per sample, and the average DNA content per cell for the cell sample. This would then allow the gene expression results to be compared in terms of the quantitative gene expression per cell, the basic functional biological unit, or compared in terms of the quantitative gene expression per haploid or diploid DNA content for the compared cells. Both comparison methods have utility for analysing and interpreting gene expression results and gene expression comparison results. For simplicity, this document will emphasize the gene expression activity per cell approach. However, converting from this approach to the gene activity per diploid DNA content is straightforward.

There are a variety of widely used methods which can be used for detecting and quantitating the amount of DNA and RNA in cells or cell samples (7, 8, 13, 15, 200, 201, 202). These include, but are not limited to spectrophotometric colorimetric, fluorescent, and PCR based methods. In a similar vein, the measurement of either the amount of total protein in a cell sample, or the amount of a particular protein fraction in a cell sample, can be used to determine the number of cells present in a cell sample, if the amount of total cell protein per cell, or the amount of the particular protein fraction per cell is known for each cell sample. There are a variety of widely used methods for detecting and quantitating the amount of total protein, or a particular protein, in a cell sample, or per cell (202).

Unlike DNA, the total RNA content per cell and total mRNA content per cell, often varies greatly from cell type to cell type, and even differs significantly for the same cells in different growth stages. In general there is a scarcity of available information concerning the absolute amount of total RNA per cell or total mRNA per cell, or the relative amounts of total RNA per cell or total mRNA per cell, for the same cells under different conditions and for different types of cells under different conditions of maintenance or growth or treatment. Therefore, the total RNA content or total mRNA content of a cell sample does not have general utility for determining cell numbers, but may have utility for this purpose in particular well characterized situations where the absolute and/or relative total RNA content per cell, or total mRNA content per cell is known for particular cells which are being compared. In this situation the absolute number of analyzed cells is equal to, (the amount of a particular cell sample's total RNA or total mRNA which is present in the analysis)÷(the total RNA content per sample cell or the total mRNA content per sample cell). In this situation one of skill in the art will recognize that the relative number of cells compared can also be determined from the known values for total RNA content per cell, or total mRNA content per cell. There are a variety of widely used methods for detecting and quantitating the amount of total RNA, or total PA mRNA, in a cell sample, or per cell (7, 8, 13, 15, 200, 201, 202, 203).

As discussed in the later section on the determination of total RNA per cell, prior art values for the total RNA per cell and total mRNA per cell are often underestimated. If such an underestimated total RNA per cell or total mRNA per cell value is used to determine the number of cells represented by a given amount of total RNA or total mRNA, the resulting cell number value will be an overestimate.

Each method for determining the absolute number of cells in a cell sample, or the relative number of cells from compared cell samples has a level of quantitative accuracy associated with it. The accuracy of cell number determination required by a particular cell sample gene expression analysis, or by a particular gene expression analysis comparison, should be considered when choosing the cell counting method if at all possible.

The sample cell ratio being compared can also be determined from the results of a microarray, or non-microarray, gene expression analysis, if a certain key requirement can be met. This can be done by using one or more mRNA's, which are naturally present in the cell samples, as an internal control. A variety of mRNA types, including housekeeping gene mRNA's can be used for this purpose if certain specific requirements can be met. Each mRNA transcript utilized as a housekeeping gene internal control must be naturally present in the mRNA of one, more than one, or all, of the cell samples being compared. The key requirement for the valid use of internal control mRNA transcripts for the purpose of determining the sample cell ratio being compared, is that a quantitatively accurate measure of the extent of expression for at least one mRNA transcript type must be known for each different cell sample being compared. Here the term, extent of mRNA expression refers to the number of, or average number of, mRNA transcripts per cell in a sample. It is not necessary to know the absolute number of mRNA transcripts per cell for the control gene in order to utilize its mRNA as a valid internal control. In this context, either the accurate ratio of, (the extent of mRNA expression for one gene in one sample)÷(the extent of mRNA expression of the same gene in another sample); or (the extent of mRNA expression for one gene in one sample)÷(the extent of mRNA expression for a different gene in a different sample); qualifies as an accurate quantitative measure of the extent of expression for the mRNA transcripts involved. Note that in order to identify the existence of such internal control mRNA transcripts it is necessary to perform gene expression analyzes which require determining a quantitative measure of the number of cells present in each sample analyzed. It is not known whether such internal control mRNA transcripts actually exist in different cell samples since the practice of the invention is necessary in order to accurately identify such internal control mRNA transcripts, and an effort to identify such internal control mRNA transcripts using the method of the invention has not as yet been known to occur.

Determination of Total RNA/Cell and Total mRNA/Cell for Cells or Cell Samples.

As discussed above, established methods exist for determining the relative or absolute number of cells in a cell sample. A variety of methods also exist for determining the relative or absolute amount of total RNA in a cell sample. Herein the total RNA is termed the T-RNA of a cell sample. These methods include, but are not limited to, the following. Methods for determining the relative number of cells and the relative amount of T-RNA present in intact cells by using flow cytometry and quantitative differential dye staining of total cell RNA and DNA (205). Methods for determining the T-RNA content and/or DNA content of cell sample lysates which do not require cell sample nucleic acid purification (15). Methods for determining the T-RNA content and/or total DNA content of cell samples which require cell sample T-RNA and/or DNA purification.

The determination of the T-RNA content per cell is almost always done by the following process. (i) The number of cells present in a cell sample are determined. (ii) The T-RNA is isolated from a known number of sample cells by standard methods. (iii) The amount of T-RNA isolated from the known number of cells is quantitated. (iv) The T-RNA per cell value for the cells is then equal to, (the amount of isolated T-RNA obtained)÷(the number of sample cells used to isolate the T-RNA). Prior art practice almost always regards this value as accurately reflecting the T-RNA per cell value, which exists in the cell sample. However, such a value can accurately reflect the true T-RNA per cell value only if the RNA isolation efficiency of the T-RNA isolation process is 100%. That is, all of the RNA present in the processed cell sample is present in the isolated T-RNA preparation. In reality, this rarely occurs, and it is not uncommon to lose a significant portion of the cell sample RNA in the isolation process. If a significant loss occurs, then a value for T-RNA per cell, which is based on the amount of isolated RNA obtained, will be an underestimate. Consequently, if such an underestimated T-RNA per cell value is used to determine the number of cells represented by a given amount of T-RNA, the resulting cell number will be overestimated by the same factor that the T-RNA per cell value is underestimated. This occurs because it is believed that for a particular cell sample T-RNA prep, even when the RNA isolation efficiency is less than 100% the R, Fmole, and Fmass, assumptions are valid for the T-RNA prep. Prior art microarray and non-microarray gene expression analysis practice rarely determines the T-RNA per cell of intact cell samples, or the efficiency of extraction of RNA from compared cell samples.

The RNA isolation efficiency for a cell sample can be determined by determining the value for the amount of T-RNA or mRNA per intact sample cell, and then determining the value for the amount of isolated T-RNA or mRNA per cell obtained by isolating and quantitating the amount of T-RNA or mRNA from a known number of the same sample cells. The ratio of, (the amount of isolated RNA per cell)÷(the amount of RNA per intact cell), is the RNA isolation efficiency value, or the RIE value. For a gene expression comparison assay the ratio of, (one cell samples RIE)÷(the other compared cell sample RIE), is termed the RIE ratio or RIER.

As discussed earlier, prior art generally believes and practices that the total PA mRNA fraction of a cell or cell sample represents, with very minor exceptions, the total mRNA transcript population of a cell or cell sample. A variety of methods have been utilized to determine the absolute and/or relative amount of total PA mRNA in a cell sample (7, 8, 13, 148, 200). One method requires the isolation of T-RNA from the cell sample. For this method, the determination of the total mRNA per cell is almost always done by the following process. (i) T-RNA from a known number of sample cells is isolated and quantitated to obtain a T-RNA per cell value. (ii) A known amount of T-RNA is contacted with oligo dT in order to specifically isolate the total mRNA molecule fraction from the T-RNA. (iii) The amount of total mRNA isolated from the known amount of T-RNA is quantitated. (iv) The total mRNA per sample cell is then equal to, (the amount of total mRNA isolated)÷(the number of cells represented by the amount of T-RNA processed). Such a value can accurately reflect the total mRNA per cell value which exists per sample cell only if the following conditions are met. (a) The isolation efficiency of T-RNA from the cell sample is 100%. (b) The isolation efficiency of total mRNA molecules from the T-RNA is 100%. As discussed earlier, the RNA isolation efficiency of T-RNA from cell samples is often significantly less than 100%. Further, the cell sample T-RNA is often degraded, and this results in a situation where only the 3′ ends of a degraded mRNAs will be isolated by the oligo dT separation step, and only the poly (A) tract containing 3′ ends of the mRNAs which are present in the T-RNA will be present in the isolated mRNA fraction. Thus, it is not uncommon for neither condition to be met. The observed total mRNA per cell value can vary greatly, depending on these efficiencies of T-RNA and total mRNA isolation. For any situation where one or the other, or both conditions are not met, the total mRNA per cell value obtained would be an underestimate of the true value. For any situation where condition (a) is not met, the underestimated total mRNA per cell value obtained cannot be used to accurately determine the number of cells represented by a given amount of total mRNA, and the resulting cell number will be underestimated. For a situation where (a) is met, but because of RNA degradation (b) is not, the underestimated total mRNA per cell value obtained for a particular cell sample isolated mRNA prep, can be used to accurately determine the number of cells represented by a given amount of that particular cell sample isolated mRNA prep, if 100% of RNA molecules which possess PA are isolated. This occurs even though for the particular cell sample isolated mRNA, the amount of mRNA, which represents one cell, is less than the true mRNA per cell value for undegraded mRNA. In other words, a cell equivalent of the isolated degraded mRNA has a lower mRNA per cell value than a cell equivalent of isolated undegraded mRNA. In this situation, it is believed that for this degraded mRNA preparation the R and Fmole assumptions are valid, while the Fmass assumption is clearly invalid.

The above-described approach measured the total isolated mRNA per cell for a cell sample in terms of the mass of mRNA per cell or average cell. Another approach for determining a quantitative measure of the total mRNA per cell is to obtain a measure of the number mRNA transcripts of all kinds per cell. As discussed, it is believed that the vast majority of the mRNA molecules in a eukaryotic cell are associated with a significant poly (A) tract. Because of this the total number of mRNA molecules present in a cell sample T-RNA or isolated mRNA prep can be determined by determining the total number of individual poly (A) tracts in the RNA prep. Prior art methods are available to accomplish this (204). One method involves the quantitative saturation hybridization of labeled poly (dT) or poly (U) of known length to an amount of cell sample T-RNA or isolated mRNA, which represents a known number of cells. The measured number of poly (A) tracts is then equal to the number of mRNA molecules, which are associated with a poly (A) tract in the measured RNA. The number of poly (A) tract containing mRNA molecules per cell is then equal to, (the total number of poly (A) tract molecules present in the cell sample RNA)÷(the number of cells represented by the amount of RNA in the assay). Such a measurement can also be done on a cell lysate containing a known number of cells. Here, the number of mRNA molecules per cell which are associated with poly (A) tracts is equal to, (the total number of poly (A) tracts present in the lysed cell sample)÷(the total number of lysed cells present in the lysed cell sample). Herein, the number of mRNA molecules of all kinds per cell is termed the sample cell total mRNA molecules per cell or the STM. Since it is believed that the R and Fmole assumptions are valid for isolated cell sample T-RNA and mRNA, then the STM value should be the same for a particular cell sample, a T-RNA isolated from the particular cell sample, and the total mRNA isolated from the T-RNA. This should occur whether the cell RNA is degraded or undegraded.

Prokaryotic mRNA transcript molecules are not associated with significant poly (A) tracts, and the above-described methods are not applicable for these cells. Currently, there is no direct method for determining the total mRNA per cell for prokaryotes.

Determination of SCR for a Cell Sample Gene Expression Comparison Assay. The Direct Comparison of Sample Cell RNAs.

Only certain gene expression comparison methods, such as northern blot, dot blot, nuclease protection, certain ELISSA assays, and rarely microarrays, directly compare cell sample T-RNA or isolated mRNA in an assay. For these methods, direct comparison of RNAs indicates that an aliquot of each compared cell sample's T-RNA or mRNA is incorporated directly into the assay hybridization solution. For such an assay the sample cell number ratio or SCR, is equal to (the number of sample cells represented by the amount of cell T-RNA or mRNA from one cell sample which is present in the assay hybridization solution)÷(the number of sample cells represented by the amount of cell T-RNA or mRNA from a different cell sample which is present in the assay hybridization solution). Herein, the amount of cell sample T-RNA or isolated mRNA which is equivalent to one cell or one average sample cell, is termed a cell equivalent or CE of T-RNA, or a cell equivalent or CE of mRNA. In this context the SCR of a gene comparison assay is equal to, (the number of T-RNA CEs or mRNA CEs from one cell sample which is present in the assay hybridization solution)÷(the number of T-RNA CEs or mRNA CEs from a different compared cell sample which is present in the assay hybridization solution). The assay CE value can be affected by the state of degradation of the T-RNA or isolated mRNA. This will be discussed below. For this discussion it will be assumed, as does the prior art microarray practice, that the R and Fmole assumptions are valid for the 3′ end portions of the cell sample mRNAs.

Table 39 presents the definition of CE for different cell sample RNA preparations. For an undegraded or degraded isolated cell sample T-RNA prep, even when the T-RNA isolation efficiency is less than 100% the T-RNA CE value is equal to the amount of total RNA present in one intact sample cell. For an mRNA prep isolated from an undegraded cell sample T-RNA prep, the mRNA CE value is equal to the amount of mRNA of all kinds present in one intact sample cell or average sample cell, even when the mRNA isolation efficiency is less than 100%. However, the mRNA CE value for an mRNA prep isolated from degraded cell sample T-RNA is not equal to the CE value for the mRNA in an intact sample cell. Herein, such an isolated degraded mRNA is termed a DI-mRNA. Here, the DI-mRNA CE will vary, depending on the degree of degradation of the cell sample T-RNA prep used to produce the DI-mRNA.

TABLE 39
Definition of CE Values for Cell Sample RNAs

| | | Definition of Cell Equivalent (CE) | |
|---|---|---|---|
| Type of Cell Sample RNA | | of Cell Sample RNA | |
| (1) | Undegraded or degraded | (1) | The amount of total RNA per intact cell for the |
| | T-RNA in intact cell sample | | cell sample. |
| (2) | Undegraded or degraded | (2) | As (1)* |
| | T-RNA isolated from cell | | |
| | sample | | |
| (3) | Undegraded or degraded | (3) | The amount of mRNA of all kinds per intact cell |
| | mRNA in intact cell sample | | for the cell sample. |
| (4) | Undegraded or degraded | (4) | As (3)* |
| | mRNA present in T-RNA | | |
| | isolated from cell sample | | |
| (5) | Undegraded mRNA | (5) | As (3)* |
| | isolated from undegraded | | |
| | cell sample T-RNA | | |
| (6) | Degraded mRNA isolated from degraded cell sample T-RNA | (6) | $\frac{\begin{matrix}( CE of mRNA in intact cell ) \times \\ ( average nucleotide length of isolated \\ degraded cell sample mRNA )\end{matrix}}{ \begin{matrix}( average nucleotide length of isolated \\ undegraded cell sample mRNA )\end{matrix}}$ |

*The CE value even when the cell sample T-RNA or mRNA isolation efficiency is less than 100%.

This occurs because the cell sample isolated mRNA prep consists of only those mRNA 3′ end fragments in the T-RNA prep which have a poly (A) tract attached. The nucleotide length of such poly (A) tract associated mRNA fragments will depend on the degree of degradation of the cell sample T-RNA. For such a cell sample DI-mRNA prep, the amount of DI-mRNA which is present in, or represents the mRNA population of one cell, is less than the amount of undegraded mRNA present in one cell. How much less depends on the degree of degradation of the T-RNA. A CE of this DI-mRNA would represent a smaller amount of mRNA, than the CE for an undegraded mRNA prep, or a less degraded mRNA prep. This can be illustrated by considering the following idealized situation. (i) The amount of mRNA of all kinds per intact cell for a cell sample is 1 picogram (1 Pg), and, therefore, the mRNA CE for an isolated undegraded mRNA prep from the cell sample, is 1 Pg. (ii) The T-RNA from the cell sample is degraded in such a way that the average nucleotide length of the DI-mRNA prep is one half of the average nucleotide length of an undegraded mRNA prep from the same cell sample. (iii) Because of ii, the average amount of mRNA per cell, which is isolated from the DI-mRNA is not 1 Pg per cell, but about 0.5 Pg per cell, since the DI-mRNA CE is equal to the product of, (the fraction of the mass of the total cell sample mRNA present in the degraded T-RNA prep which is isolated as PA mRNA)×(1 Pg of mRNA per cell). In other words, a DI-mRNA CE is equal to the mass in one cell of the 3′ end portions of the cell mRNA molecules which are directly represented in the DI-mRNA prep. For such a cell sample DI-mRNA the R and 3′ end mRNA portion Fmole assumptions are valid, but the Fmass and 5′ end mRNA portion Fmole assumptions are not valid. In such a situation, a microarray assay must be designed to detect the 3′ end mRNA portions.

For northern blot, dot blot, nuclease protection, certain ELISSA, and microarray assays which directly compare cell sample degraded or undegraded T-RNA preps, the assay T-RNA CE value for a compared cell sample is equal to the amount of T-RNA present in one intact cell or average cell, of the cell sample. The number CEs of T-RNA in the assay is equal to the ratio of, (the mass of cell sample T-RNA present in the assay hybridization solution)÷(the cells sample T-RNA CE value). The determination of the mass of T-RNA per cell for a cell sample was described in an earlier section. Well established procedures exist for determining the amount of nucleic acid of any kind. Here, the assay SCR value is equal to the ratio in the assay hybridization solution of, (the number of T-RNA CEs for once cell sample)÷(the number of T-RNA CEs for the other compared cell sample).

For northern blot, dot blot, nuclease protection, CERTAIN ELISSA, and microarray assays, which directly compare cell sample undegraded mRNA preps, the assay mRNA CE value for a compared cell sample, is equal to the amount of mRNA of all kinds present in one cell, or average cell, of the cell sample. The number of mRNA CEs in the assay is equal to the ratio of, (the mass of cell sample mRNA present in the assay hybridization solution)÷(the cell sample mRNA CE value). The determination of the mass of mRNA of all kinds per cell was described in an earlier section. Here, the assay SCR value is equal to the ratio in the assay hybridization solution of, (the number of isolated mRNA CEs for one cell sample)÷(the number of isolated mRNA CEs for the other cell sample).

For northern blot, dot blot, nuclease protection, certain ELISSA, and microarray assays, which directly compare cell sample DI-mRNA preps, the assay DI-mRNA CE value for a compared cell sample is equal to, the product of (the fraction of the mass of mRNA present in the T-RNA prep which is isolated as PA mRNA)×(mass of mRNAs of all kinds per cell for the degraded T-RNA prep). The number of DI-mRNA CEs in the assay is equal to the ratio of, (the mass of cell sample DI-mRNA present in the assay hybridization solution)÷(the cell sample DI-mRNA CE value). The determination of the amount of mRNA present in an assay hybridization solution is straightforward. Here the assay SCR value is equal to the ratio in the assay hybridization solution of, (the number of DI-mRNA CEs for one cell sample)÷(the number of DI-mRNA CEs for the other cell sample). For the determination of this SCR it is necessary to determine the number of CEs of each compared cell sample's DI-mRNA, which are present in the assay hybridization solution. To do this it is necessary to determine the DI-mRNA CE value for each cell sample's DI-mRNA prep. Obtaining the CE value for a particular cell sample's DI-mRNA prep can be complex and problematic. This is discussed below.

One approach to determining the CE of a cell sample DI-mRNA requires knowing the intact sample cell mRNA CE, and the fraction of cell sample undegraded T-RNA which consists of mRNA, and then determining the fraction of cell sample degraded T-RNA which consists of DI-mRNA. The cell sample DI-mRNA CE is then equal to, (the fraction of the cell sample degraded T-RNA which consists of DI-mRNA÷the fraction of the cell sample undegraded T-RNA which consists of mRNA)×(CE of intact cell sample mRNA). The method for determining the fraction of total RNA which, consists of mRNA was earlier described.

Another approach for determining the CE value for a cell sample DI-mRNA prep requires knowing the average undegraded mRNA nucleotide length, and the undegraded mRNA nucleotide length distribution profile for the cell sample mRNA prep of interest. In addition, it is necessary to assume that the degradation process which produced the degraded mRNA is random in nature, and affects most of the mRNA molecules in the same random manner, and that the R and Fmole assumptions are valid for at least the cell sample DI-mRNAs 3′ end portions. The determination of the average mRNA nucleotide length and nucleotide length distribution in a cell sample T-RNA or isolated mRNA prep, is discussed later. A measure of the CE value for a cell sample DI-mRNA prep can be obtained from the difference in the average mRNA nucleotide length values of the undegraded and degraded cell sample mRNA molecule populations. As an example, if an analysis of the mRNA nucleotide length and nucleotide length distribution profiles indicates that the average nucleotide length of the DI-mRNA is about 0.5 that of the undegraded mRNA, then the average DI-mRNA 3′ end mRNA molecule has about 0.5 the mass of the average undegraded cell sample mRNA molecule. This would indicate that only about one half of the total mass of mRNA present in the cell sample T-RNA prep can be isolated as DI-mRNA. The resulting DI-mRNA CE value would then be one half of the mRNA CE value for the T-RNA and the cell. The determination of the mRNA CE for a cell sample, i.e., the mass of mRNA per cell, was discussed earlier. Note that for some cell samples, it may not be possible to isolate T-RNA or mRNA which is known to be undegraded, and it is not possible to determine the average nucleotide length of the undegraded mRNA molecules. For such cell samples this approach cannot be used. In such an event a related approach can be used to obtain a measure of the cell sample undegraded mRNA average nucleotide length and nucleotide length distribution profile. For simplicity, this will be discussed in terms of the total mRNA molecule populations of mammalian cells. Existing evidence indicates that the average nucleotide lengths, and nucleotide length distribution profiles, for different mammalian undegraded mRNA preps are similar. It is generally believed that the average nucleotide length for a typical mammalian cell is about 2000 nucleotides, and the nucleotide distribution profile is similar for many different mammalian isolated mRNA preps believed to be undegraded. In such a situation, the generic mammalian cell sample average nucleotide length value and nucleotide length distribution profile, can be used to determine the CE value of the DI-mRNA as described above.

For northern blot, dot blot, nuclease protection, certain ELISSA, and microarray assays, which directly compare a cell sample undegraded isolated mRNA prep, and a cell sample DI-mRNA prep the assay SCR value is equal to the ratio in the hybridization solution of, (the number of undegraded mRNA CEs for one cell sample)÷(the number of DI-mRNA CEs for the other cell sample).

Determination of SCR for a Cell Sample Gene Expression Comparison Assay Involving the Direct Comparison of Cell RNA Equivalents Such as cDNA or cRNA.

A large variety of reverse transcriptase (RT) related gene expression analysis methods directly compare cell RNA equivalents such as cDNA or cRNA in an assay. These include but are not limited to, microarray methods, various forms of RT-PCR methods, various forms of differential display methods, various forms of representational difference analysis methods, SAGE, and others (7, 8). For these methods, direct comparison of cDNA or cRNA indicates that an aliquot of each compared cell sample's cDNA prep or cRNA prep is incorporated directly into the assay, as for example into a microarray assay hybridization solution, or into a PCR amplification solution. This will be discussed below. For simplification, the discussion will be in terms of the widely used microarray and RT-PCR methods. However, the discussion will broadly apply to other reverse transcriptase (RT) related methods. For these assays, the assay SCR is equal to the ratio of (the number of sample cells represented by the amount of one cell sample's RNA equivalents which is present in the microarray assay hybridization solution, or in the PCR amplification solution)÷(the number of sample cells represented by the amount of the other cell sample's RNA equivalents which is present in the microarray assay hybridization solution, or in the PCR assay amplification solution). Herein, the amount of cell sample cDNA or cRNA, which is equivalent to one cell or one average sample cell, is termed a cell equivalent, or CE of cDNA, or a CE of cRNA. In this context the SCR of a gene comparison assay is equal to, (the number of cDNA CEs or cRNA CEs from one cell sample which is present in the microarray assay hybridization solution or the PCR assay amplification solution)÷(the number of cDNA CEs, or cRNA CEs, from the other cell sample which is present in the microarray assay hybridization solution or the RT-PCR assay amplification solution). In order to determine the number of a cell sample's cDNA or cRNA CEs in an assay, it is necessary to know; (a) The amount of cell sample cDNA or cRNA present in the microarray assay hybridization solution or RT-PCR assay amplification solution, and; (b) The CE value for the cell sample cDNA or cRNA prep. The determination of the amount of cell sample cDNA or cRNA produced, and the amount of cell sample cDNA or cRNA or cRNA present in a microarray assay hybridization solution, or an RT-PCR assay amplification solution, is straightforward, if there is enough cell sample cDNA or cRNA synthesized to quantitate accurately. This is often not the case, as for example, the analysis of laser capture and needle biopsy cell samples. Methods for such determination of nucleic acid amounts were described earlier. The determination of cell sample T-RNA and mRNA CE values was also described earlier. Methods for determining the nucleotide length of RNA, cDNA, and cRNA preps are described later. The determination of cell sample cDNA or cRNA prep CE values may not be straightforward. This is discussed below. The subject of microarray assay cDNA or cRNA CE values is discussed first, followed by a discussion of RT-PCR cDNA CE values.

For these discussions it will be useful to describe prior art microarray and RT-PCR practices with regard to the determination of cell sample cDNA prep and cRNA prep CE values, and the determination of the assay SCR values for assay comparisons of cell sample cDNA or cRNA preps. Such descriptions are summarized in Tables 40, 41, 42, 43.

TABLE 40
Prior Art Practices with Regard to Microarray Assays Using First Strand
Synthesized cDNA.
Prior Art Practice:

| | |
|---|---|
| (1) | Rarely determines the CE value for intact cell sample T-RNA or mRNA. |
| (2) | Seldom determines the average nucleotide length of undegraded cell sample T-RNA or |
| | mRNA. |
| (3) | Rarely determines the isolation efficiency of T-RNA or mRNA from a cell sample. |
| (4) | Generally does not determine the isolated cell sample T-RNA or mRNA state of |
| | degradation. |
| (5) | Generally determines the amount of cell sample T-RNA or mRNA used in the RT step. |
| (6) | Does not determine the CE value for the isolated cell sample T-RNA or mRNA used in |
| | the RT step. |
| (7) | Rarely determines the amount of cell sample cDNA produced in the first strand |
| | synthesis RT step. |
| (8) | Rarely determines the first strand cDNA prep average nucleotide length. |
| (9) | Does not determine the CE value for the cell sample first strand cDNA prep. |
| (10) | Rarely determines the amount of first strand cell sample cDNA prep present in the |
| | microarray |
| | assay hybridization solution. |
| (11) | Does not determine the number of cell sample first strand cDNA CEs present in the |
| | microarray |
| | assay hybridization solution. |
| (12) | Does not determine the assay SCR value present in the microarray assay hybridization |
| | solution |
| | for cell sample cDNA prep comparisons. |
| (13) | Does not take the assay SCR value into consideration during the normalization and |
| | interpretation of assay measured DGER results. |

TABLE 41
Prior Art Practice with Regard to Microarray Assays Using cRNA
Prior Art Practice:

| | |
|---|---|
| (1) | Rarely determines the CE value for intact cell sample T-RNA or mRNA. |
| (2) | Seldom determines the average nucleotide length of undegraded cell sample T-RNA or |
| | mRNA. |
| (3) | Rarely determines the isolation efficiency of T-RNA or mRNA from a cell sample. |
| (4) | Generally does not determine the isolated cell sample T-RNA or mRNA state of |
| | degradation. |
| (5) | Generally determines the amount of cell sample T-RNA or mRNA used in the RT step. |
| (6) | Does not determine the CE value for the isolated cell sample T-RNA or mRNA used in |
| | the RT step. |
| (7) | Rarely determines the amount of cell sample cDNA produced in the first strand |
| | synthesis RT step. |
| (8) | Rarely determines the first strand cDNA prep average nucleotide length. |
| (9) | Does not determine the CE value for the cell sample first strand cDNA prep. |
| (10) | Rarely determines the amount of first strand cDNA put into the second strand synthesis |
| | step. |
| (11) | Rarely determines the amount of double strand cell sample cDNA produced in the |
| | second strand synthesis step. |
| (12) | Rarely determines the average nucleotide length of the synthesized double strand cell |
| | sample cDNA. |
| (13) | Does not determine the CE value for the cell sample double strand cDNA prep. |
| (14) | Rarely determines the amount of cell sample double strand cDNA put into the cRNA |
| | synthesis step. |
| (15) | Often determines the amount of cRNA produced in the cDNA synthesis step. |
| (16) | Occasionally determines the average nucleotide length of the synthesized cell sample |
| | cRNA prep. |
| (17) | Does not determine the CE value for the cell sample cRNA prep. |
| (18) | Generally determines the amount of cRNA present in the microarray assay hybridization |
| | solution. |
| (19) | Does not determine the number of cell sample cRNA prep CEs present in the assay |
| | hybridization solution. |
| (20) | Does not determine the assay hybridization solution SCR value for compared cell |
| | sample cRNA preps. |
| (21) | Does not take the assay SCR value into consideration during the normalization and |
| | interpretation of assay measured DGER results. |

TABLE 42
Prior Art RT-PCR Practices with Regard to RT-PCR Assays Using Oligo dT
or Random Primer or Certain SG Primed Cell Sample cDNA Preps
Prior Art Practice:

| | |
|---|---|
| (1) | Rarely determines the CE value for intact cell sample T-RNA or mRNA. |
| (2) | Seldom determines the average nucleotide length of undegraded cell sample T-RNA or |
| | mRNA. |
| (3) | Rarely determines the isolation efficiency of T-RNA or mRNA from a cell sample, and |
| | does not determine the cell sample intact CE value. |
| (4) | Generally does not determine the state of degradation of isolated cell sample T-RNA or |
| | mRNA. |
| (5) | Generally determines the amount of T-RNA or mRNA used in the RT step. |
| (6) | Does not determine the CE value for the isolated cell sample T-RNA or mRNA used in |
| | the RT step. |
| (7) | Rarely determines the amount of standard or cell sample cDNA produced in the RT |
| | step. |
| (8) | Rarely determines the standard or cDNA prep average nucleotide length. |
| (9) | Does not determine the CE valued for the synthesized standard or cell sample cDNA |
| | prep. |
| (10) | Rarely determines the amount of standard or cell sample cDNA present in the PCR |
| | amplification solution. |
| (11) | Often assumes that the AE R and AE Fmole assumptions are valid for the cell sample |
| | cDNA prep for |
| | at least a portion of each particular gene mRNA which is present in the cell sample |
| | mRNA |
| | prep. If these assumptions are valid then, in effect, the assay AE·SE value for each |
| | particular |
| | gene cDNA is equal to one. |
| (12) | Does not determine the number of cell sample cDNA CEs present in the assay PCR |
| | amplification solution. |
| (13) | Does not determine the assay SCR value associated with the assay PCR amplification |
| | step for a cell sample cDNA prep comparison. |
| (14) | Does not take the assay SCR value into consideration during the normalization and |
| | interpretation of the RT-PCR assay measured particular gene RASR value. |
| (15) | Only rarely determines for each particular external standard used in the assay, the |
| | AE·SE assay value for one cell sample or compared cell samples. |
| (16) | Often does not determine for each particular gene of interest, or each particular external |
| | or internal standard in the assay, the AE·AE assay value for one cell sample or |
| | compared cell samples. |

TABLE 43
Prior Art Practices with Regard to RT-PCR Assays Using Cell Sample cDNA
Preps Produced Using Only One or a Few SG Primers.
Prior Art Practice:

| | |
|---|---|
| (1) | Rarely determines the CE value for intact cell sample T-RNA or mRNA. |
| (2) | Seldom determines the average nucleotide length of undegraded cell sample T-RNA or |
| | mRNA. |
| (3) | Rarely determines the isolation efficiency of T-RNA or mRNA from a cell sample, and |
| | does not determine the cell sample intact CE value. |
| (4) | Generally does not determine the state of degradation of isolated cell sample T-RNA or |
| | mRNA. |
| (5) | Generally determines the amount of T-RNA or mRNA used in the RT step. |
| (6) | Does not determine the CE value for the isolated cell sample T-RNA or mRNA used in |
| | the RT step. |
| (7) | Rarely determines the amount of cell sample particular gene cDNA which is produced |
| | in the RT step, or which is present in the assay PCR amplification solution. |
| (8) | Rarely determines a cell samples particular gene cDNA AE·SE assay value or the assay |
| | value for each external standard, or internal standard or exogenous standard used in the |
| | assay, for one cell sample or compared cell samples. |
| (9) | Rarely determines the cell sample particular gene cDNA average nucleotide length. |
| (10) | Cannot directly determine the number of cell sample cDNA ACEs which are produced |
| | in the RT step, or which are present in the assay PCR amplification solution. |
| (11) | Often does not determine for each particular external standard, internal standard, |
| | exogenous standard, or particular gene of interest in the assay, the AE·AE value for one |
| | cell sample or compared cell samples. |
| (12) | Rarely takes into consideration during the normalization process the pertinent values for |
| | the particular gene and standard AE·SE. |
| (13) | Does not determine the assay SCR value for the PCR amplification step and therefore |
| | does not normalize the assay results for the SCR value. |
| (14) | Cannot know that the assay values for the particular gene mRNA transcript number or |
| | mRNA abundance values are correct. |

Determination of Microarray Assay cDNA or cRNA CE Values and SCR Values.

The microarray CE value for a cell sample cDNA or cRNA prep, can be affected by a variety of assay factors. These include, but are not limited to, the following. (i) The state of degradation of the cell sample T-RNA or mRNA. (ii) Whether T-RNA or isolated mRNA is used to produce the cell sample cDNA or cRNA preps. (iii) The type of primer used for the cDNA or cRNA synthesis. (iv) The nucleotide length of the synthesized cDNA or cRNA relative to the nucleotide length of the RNA template used to produce the cDNA. This ratio has been defined as the cDNA length ratio or CLR. (v) The isolation efficiency of T-RNA and mRNA from the cell sample. (vi) The average nucleotide length of the cell sample synthesized cDNA or cRNA prep. (vii) The efficiency of cDNA synthesis for a cell sample cDNA or cRNA prep. (viii) The efficiency of synthesized cDNA or cRNA recovery for further use. It will be useful to first discuss the effect of factors (i)-(iv) on the cDNA or cRNA value of a microarray assay, and then discuss the other factors. This discussion will assume, as does the prior art microarray practice, that the R and Fmole assumptions are valid for the cell sample cDNA or cRNA preps, for at least the 3′ end portions of cell sample RNAs. Initially this discussion will focus on the determination of cell sample cDNA CE values. The discussion is also directly applicable to the determination of cRNA CE values, since the process of producing a cRNA prep first requires the synthesis of cDNA from cell sample RNA. Determination of cRNA CE values will be discussed in more detail later. These discussions will emphasize oligo dT and random primers.

Table 44 summarizes the effect of assay factors (i)-(iv) on a cell sample cDNA prep CE value. Table 44 relates the CE value for the RNA type used to produce the cDNA to the synthesized cDNA prep CE value. Oligo dT priming produces cDNA which represents only PA mRNA whether the cDNA is produced from undegraded T-RNA or isolated mRNA, or degraded T-RNA, or isolated mRNA. Herein, degraded isolated mRNA is termed DI-mRNA, and degraded T-RNA is termed DT-RNA. As indicated in Table 44, when oligo dT is used to produce cDNA from undegraded T-RNA or purified mRNA, and the CLR is equal to one, then the cDNA CE value equals the CE value of the mRNA which is used to produce it. However, when the CLR value does not equal one, then the cDNA CE value does not equal the CE value of the mRNA used to produce it. When oligo dT is used to produce cDNA from DT-RNA, then the cDNA CE value is not equal to the CE value of the mRNA which is used to produce it, even when the CLR is equal to one.

TABLE 44
Effect of Assay Factors on Cell Sample cDNA Prep CE Value

| | | | | | RNA Type | |
|---|---|---|---|---|---|---|
| RNA | ^{(a)}RNA | ^{(b)}(CE of Isolated RNA Sample) | | | Represented | (CE of cDNA) |
| Prep | Integrity | (Maximum CE for RNA) | ^{(c)}Primer | CLR | by cDNA | (̅C̅E̅ o̅f̅ R̅N̅A̅ U̅s̅e̅d̅ t̅o̅ P̅r̅o̅d̅u̅c̅e̅ c̅D̅N̅A̅)̅ |
| T-RNA | UD | 1 | odT | 1 | mRNA | 1 |
| | | | | <1 | | <1 |
| | D | 1 | odT | 1 | mRNA | <1 |
| | | | | <1 | | <1 |
| Isolated | UD | 1 | odT | 1 | mRNA | 1 |
| mRNA | | | | <1 | | <1 |
| | D | <1 | odT | 1 | mRNA | 1 |
| | | | | <1 | | <1 |
| T-RNA | UD | 1 | Random | <1 | rRNA | ˜1 |
| | D | 1 | Random | <1 | mRNA | ˜1 |
| | | | | | etc. | |
| Isolated | UD | 1 | Random | <1 | rRNA | ˜1 |
| mRNA | D | <1 | Random | <1 | mRNA | ˜1 |
| | | | | | etc. | |

^{(a)}UD = Undegraded RNA.
^{(b)}Maximum CE for RNA refers to the CE value for the RNA sample in intact sample cells.
^{(c)}odT = oligo dT primer.

This occurs because the DI-mRNA molecules which are present in the DT-RNA and which can be primed by oligo dT, consists of only the 3′ end portion of each mRNA. When oligo dT is used to produce cDNA from DI-mRNA, and the CLR value is equal to one, then the cDNA CE value is equal to the CE value of the DI-mRNA used to produce it. However, when the CLR is not equal to one, the cDNA CE value is not equal to the CE value of the DI-mRNA used to produce it. For a situation where random primed cDNA is produced from undegraded or degraded T-RNA, the cDNA represents all of the different RNA types present in the T-RNA. Here the cDNA CE value is essentially equal to the CE value of the T-RNA prep used to produce it. When random primed cDNA is produced from undegraded or degraded isolated mRNA, the cDNA CE is essentially equal to the CE of the undegraded or degraded isolated mRNA prep used to produce it. Thus, when the CE of the cell sample RNA is determined, the CE of the random primed cDNA is also determined.

As indicated in Table 44, when oligo dT primer is used, the produced cDNA prep CE value equals the CE of the isolated mRNA or the mRNA in T-RNA, only when the cDNA synthesis CLR value equals one. Since the CLR value for the oligo dT primed production of cDNA from undegraded T-RNA or isolated mRNA, is almost always significantly less than one, then only rarely does a prior art cell sample cDNA prep CE value equal the CE value for the cell sample undegraded or degraded mRNA which is present in T-RNA or isolated mRNA. Only rarely then, does a cell sample cDNA prep CE value equal the CE value of the T-RNA or mRNA which is present in the intact cells, or the CE value of the isolated cell sample T-RNA or mRNA. This is due to the following oligo dT priming related situation.

For the production of oligo dT primed cDNA preps from undegraded T-RNAs or isolated mRNA preps, the CLR value is almost always significantly less than one, and the synthesized cDNA prep does not contain a 5′ end portion of each template mRNA molecule. Further, for the oligo dT primed cDNA preps from degraded cell sample T-RNA or isolated mRNA preps, the CLR is almost always significantly less than one, and the 5′ end portion of the template mRNA molecules are not attached to a poly (A) tract, and do not serve as a template for cDNA synthesis, and are therefore not represented in the cDNA. As a consequence, for virtually all prior art oligo dT primer produced cell sample cDNA or cRNA preps, the CE value is significantly less than the CE value for cell sample T-RNA or mRNA in intact sample cells, or in isolated T-RNA or mRNA. The determination of these cDNA or cRNA prep CE values will be discussed later.

As further indicated in Table 44, random primer produced cDNA preps for degraded or undegraded cell sample T-RNA preps, or for an undegraded cell sample isolated mRNA prep, have cDNA prep CE values which essentially equal the CE value of the RNA sample used to produce the cDNA. Here, the cDNA CE value is essentially equal to either the amount T-RNA per intact sample cell, or the amount of total mRNA per intact sample cell. In order for this to be true the R, Fmole, and Fmass assumptions, must be valid for these random primed cell sample cDNA preps, for each different RNA type which is present in the T-RNA or isolated mRNA. Prior art generally believes and practices that random primed RNA templates produce cDNA preps, which are essentially completely representative of the RNA templates used to produce them.

Table 44 also indicates that a random primer produced cDNA prep for a degraded cell sample isolated mRNA, has a cDNA prep CE value which is essentially equal to the CE value of the cell sample DI-mRNA used to produce it. This DI-mRNA prep CE value is smaller than the total undegraded mRNA CE value for the intact sample cells.

A cDNA or cRNA prep which is produced from oligo dT or specific gene primed cell sample degraded or undegraded T-RNA or isolated mRNA, or from random primed degraded or undegraded cell sample isolated mRNA, is believed to represent only cell sample mRNA templates. A cDNA prep produced from random primed degraded or undegraded cell sample T-RNA however, is believed to represent essentially all of the different RNA types which are present in T-RNA, including mRNA and rRNA, siRNA, miRNA, snoRNA, and others. For a typical cell sample T-RNA prep, on a mass basis roughly 0.9 of the T-RNA represents rRNA, and 0.02 of the T-RNA represents mRNA. If it is assumed that these proportions are also present in the random primed T-RNA cDNA prep, the fraction of the random primed T-RNA which represents mRNA is small, about 0.02.

The above indicates that depending on the assay situation, a cell sample cDNA or cRNA CE: (a) is defined differently; (b) can be associated with different assay values; (c) often does not equal the CE of the cell sample T-RNA or isolated mRNA used to produce it.

Table 44 indicates that the definition of the CE can be different for different assay situations. Table 45 presents the definition of the cDNA prep CE for different assay situations. It will be useful to illustrate Table 45 definition (a) by considering the following idealized situation for an isolated cell sample mRNA. (i) In the mRNA prep all mRNA molecules are undegraded, and the average nucleotide length of a mRNA molecule is 2000 nucleotides, and the mass per cell of mRNA molecules of all kinds is 1 picogram (1 Pg). (ii) The average nucleotide length of the cDNA prep produced from the mRNA is 1000 nucleotides, and on average each cDNA molecule in the cDNA prep is half the nucleotide length of the mRNA template which produced it. As a result, each cDNA molecule in the cDNA prep represents only the 3′ end of the template mRNA which produced it, and on average half of each mRNA nucleotide sequence is not represented in the cDNA prep.

TABLE 45
Different Definitions of CE for Cell Sample cDNA Preps

| Type of Cell Sample cDNA Prep | | Definition of CE | |
|---|---|---|---|
| (a) | Essentially any oligo dT primed | (a) | The *mass of cDNA prep which is equal to, the |
| | cDNA from degraded or | | mass in one cell of all of the portions of cell mRNA |
| | undegraded cell sample T-RNA | | molecules which are directly represented by a |
| | or isolated mRNA. | | nucleotide sequence in the synthesized cDNA prep. |
| | | | ^{Δ}The approximate value of this CE. |
| (b) | Random primed cDNA prep | (b) | As (a). Here, the cDNA CE is essentially equal to |
| | from degraded isolated mRNA. | | the CE of the DI-mRNA used to produce it. |
| (c) | Random primed cDNA prep | (c) | The *mass of cDNA prep which is equal to the |
| | from undegraded or degraded | | mass of T-RNA per sample cell. Here, the T-RNA |
| | T-RNA. | | CE is essentially equal to the cDNA CE.* |
| (d) | Random primed cDNA prep | (d) | The *mass of cDNA prep which is equal to the |
| | from undegraded isolated | | mass of total mRNA per sample cell. Here, the |
| | mRNA. | | mRNA CE is essentially equal to the cDNA CE. |

*In reality there is a small difference in mass between DNA and RNA molecules of identical
length. This can be corrected for if necessary.
${}^{Δ} CE = \frac{( cell sample cDNA prep nucleotide length}{\begin{matrix}cell sample undegraded mRNA prep \\ nucleotide length )\end{matrix}} \times \\ ( Cell Sample undegraded mRNA CE )$

In this situation the mRNA CE is equal to 1 Pg per cell. However, since only half of each mRNA molecule's nucleotide sequence is represented in the cDNA prep, the cDNA CE is equal to, (mRNA CE)×(0.5), or 0.5 Pg per cell. The definitions of Table 45 (c) and (d) are straightforward, and should require no further illustration. An earlier section discussed the determination of the T-RNA per cell and mRNA per cell values for a cell sample.

Prior art microarray cell sample cDNA or cRNA comparisons virtually always input known equal amounts of cell sample T-RNA or isolated mRNA, into the reverse transcriptase step of the assay. In addition, for the great majority of prior art microarray assays, oligo dT primer is used to produce the compared cell sample cDNA or cRNA preps. As discussed above, only rarely does a prior art oligo dT produced cDNA prep CE value, equal the CE value of the mRNA which is used to produce it. Further, the amount of oligo dT primed cDNA produced from a cell sample RNA is almost always significantly less than the amount of input mRNA present in the cDNA synthesis reaction. This cDNA synthesis efficiency can be different for different cell sample RNAs, and can range from 10-60%. That is, the amount of cDNA synthesized is only 0.1 to 0.6 of the amount of input mRNA. Similarly the synthesis efficiency for random primed cDNAs ranges from 25-60%.

As a consequence, for a large majority of prior art microarray cell sample cDNA and cRNA comparisons, the known equal amount of each cell sample T-RNA or mRNA which is added to the reverse transcriptase step of the assay does not accurately reflect the amount of each cell sample cDNA produced in the reverse transcriptase step of the assay. In addition, the ratio of the amounts of each cell sample's added RNA does not reflect the ratio of the amounts of compared cell sample cDNAs in the microarray assay hybridization solution. Further, neither compared cell sample cDNA prep CE value is equal to the CE value of the cell sample RNA used to produce the cDNA prep. Therefore, for the vast majority of prior art microarray cell sample cDNA or cRNA comparisons, it is not possible to use the known amount of cell sample T-RNA or mRNA used in the assay, or the CE values for these cell sample RNAs, to determine the assay SCR value for the assay hybridization step. Thus, even if the cell ratio represented by the input amounts of compared cell sample T-RNAs or mRNAs is known, the assay SCR for the compared cell sample cDNA preps which exists in the microarray assay hybridization solution cannot be known, unless further information is available. For these prior art microarray assays, in order to determine the assay SCR it is necessary to determine a quantitative measure of the number of cDNA or cRNA CEs for each compared cell sample which is present in the microarray assay hybridization solution. In order to directly determine a quantitative measure of the number of a cell sample's cDNA or cRNA CE's which are present in the assay hybridization solution, the amount of cell sample cDNA or cRNA prep present in the hybridization solution, and the cell sample cDNA or cRNA CE value must be known. Prior art microarray practice concerning these determinations is summarized in Table 40.

Prior art microarray practice rarely determines the amount of each compared cell sample cDNA present in a microarray hybridization solution, and does not determine the CE values for the compared cDNA preps, and therefore cannot determine the number of CEs of each cell sample cDNA prep which is present in the assay hybridization solution. Thus, the assay SCR values for these prior art microarray assays cannot be known. As a result, the assay SCR value for prior art microarray assay cell sample cDNA prep comparisons cannot be known, absent further information.

For the above discussion cell sample cDNA and cRNA comparisons have, for simplicity, been discussed together because the production of a cell sample cDNA prep is necessary in order to produce a cell sample cRNA prep. The use of cell sample cRNA in microarray assays is discussed in more detail below. Prior art practices concerning the use of such cRNA preps in microarray assays are summarized in Tables 40 and 41. The general process for producing a cell sample cRNA prep involves the following (7). (a) Produce cell sample T-RNA or isolated mRNA. (b) Produce first strand cDNA from the cell sample T-RNA or isolated mRNA. This is almost always done by oligo dT priming the mRNA with a specially designed oligo dT-T₇ promoter primer. The first strand cDNA prep is then virtually always associated with a cDNA CE value which is significantly smaller than the CE of the mRNA prep used to produce the cDNA. (c) The first strand single strand cDNA molecules are converted to a double strand form. Generally 70-90% of the first strand cDNA is converted to a double strand form. (d) The double strand cDNA is then used to produce large amounts of cRNA product. At this point the cell sample cRNA prep represents one round of cRNA amplification. It is known that the T₇ polymerase system which produces the cRNA which is specific for the cell sample cDNA, can simultaneously produce significant quantities of cRNA which is not specific for the cell sample cDNA templates in the reaction mixture (206). Such non-specific RNA can be longer in nucleotide length than the longest cDNA template present. For simplicity such non-specific cRNA will be termed NS-cRNA. In addition, the average overall nucleotide length of a cell sample cRNA prep is generally significantly shorter than the average nucleotide length of the cDNA used to produce it. Prior art does not determine the fraction of a cell sample cRNA prep which is composed of NS-cRNA. The presence of significant amounts of NS-cRNA in a cell sample cRNA prep complicates the determination of a cell sample cRNA prep CE value, and the determination of the amount of cell sample cDNA specific cRNA which is present in the cRNA prep. This would also occur for a cDNA prep if NS-cDNA were produced. This occurs because the nucleotide length of the cell sample cDNA specific cRNA must be known in order to determine the cRNA prep cell sample CE value, and the presence of the NS-cRNA complicates the nucleotide length determination. In addition, in order to determine the number of cell sample cDNA specific cRNA CEs which are present in the assay hybridization solution, it is necessary to know the fraction of the total cRNA prep which consists of cell sample cDNA specific cRNA, or NS-cRNA. If it is assumed, as the prior art does, that for a cell sample cRNA prep the R and Fmole assumptions are valid, or essentially valid, for at least the 3′ end portions of all cell sample particular expressed gene mRNA transcripts, then the earlier described methods for determining cell sample cDNA prep nucleotide lengths can be used to determine the cell sample cDNA specific cRNA preparation nucleotide length. For those cRNA preps containing NS-cRNA, the nucleotide length of the cell sample cDNA specific cRNA can be determined with the use of one or more different gene specific LPNs to obtain an estimate of average nucleotide length of a cell sample cDNA or cRNA molecule population. It is also important to determine the fraction of the cell sample total cRNA prep which consists of NS-cRNA in order to determine the number of cell sample cDNA specific cRNA CEs which are produced by the cRNA synthesis process, and which are present in the assay hybridization solution. Unless this NS-cRNA fraction is known and considered, the number of cell sample cRNA CEs which are present in the assay hybridization solution can be signification overestimated. One approach to determining the NS-cRNA fraction of a cell sample total cRNA prep, is to determine the fraction of the cell sample cRNA prep which can specifically hybridize to the cell sample T-RNA or mRNA. (e) Often a second round of amplification is done to produce even more cell sample cRNA. This is generally done by producing a reverse transcriptase mediated random primed first strand cDNA prep from the first round cRNA prep, and then repeating steps (c) through (d). The use of random primer for this first strand synthesis is known to result in a significant reduction of nucleotide length of the second round amplified cRNA prep, relative to the first round amplified cRNA nucleotide length. Such a reduction can be as much as 2-3 fold or more. In the absence of NS-cRNA, this would cause a further reduction in the second round cRNA CE value, relative to the CE value of first round amplified cRNA. NS-cRNA can also be produced in the second round. (f) Occasionally further rounds of amplification are done on the second round cRNA prep by repeating step (e). The cRNA CE value can be different for each subsequent round cRNA prep.

In contrast to prior art microarray practice comparing cell sample cDNA preps, prior art microarray practice almost always compares known equal amounts of different cell sample cRNA preps. However, as with prior art cell sample cDNA comparisons, prior art does not determine the CE value for each compared cell sample cRNA prep, and therefore cannot determine the assay SCR value for the compared cell sample cRNA preps which are present in the microarray assay hybridization solution.

For many prior art microarray gene comparison assays, the amount of cell sample RNA used in the assay is too small to measure directly. This often occurs for cell samples obtained by laser capture or needle biopsy methods. On occasion, equal numbers of laser captured cells from different cell samples are compared as follows. (i) Total RNA is isolated from each cell sample. (ii) The entire preparation of each cell sample's total RNA is used to produce oligo dT•T₇ primed first strand cDNA preps. (iii) The entire first strand cDNA prep for each cell sample is converted to double strand cDNA. (iv) The entire double strand cDNA prep from each cell sample is used to produce cell sample cRNA preps. (v) The entire amount of each cell sample cRNA prep is compared in the microarray assay hybridization solution. For this situation, the ratio of the number of sample cells used for the assay is equal to one. Here, the amount of T-RNA present in each cell sample is too small to measure, and the efficiency of T-RNA isolation from each cell sample is not measured or known. Therefore, the amount of each cell sample's T-RNA, which is used to produce cDNA, and the T-RNA CE value for each cell samples T-RNA cannot be measured or known. Because of this, the number of RNA CEs for each cell sample which is used to produce the cell sample cDNA preps, cannot be known, and the ratio of the number of each cell samples T-RNA CEs which are present in the reverse transcriptase step, cannot be known to equal one. Further, the cDNA synthesis efficiency, the cDNA synthesis CLR, the cDNA prep nucleotide length, and the total amount of cDNA synthesized for each cell sample cDNA prep, are not measured, and cannot be known. Because of this the number of each cell sample's cDNA CEs which are produced, and the CE value for each cell sample cDNA prep cannot be known, and the ratio of each cell sample's number of cDNA CEs which are present in the reverse transcriptase mixture cannot be known to equal the ratio of the number of cells for each cell sample used in the assay. Similarly, the second strand cDNA synthesis efficiency, the nucleotide lengths of the cell sample double strand cDNAs produced, the total amount of each cell sample double strand cDNA produced, are not measured or known. Because of this the number of each cell sample's double strand cDNA CEs which are produced, and the CE value for each cell sample double strand cDNA prep cannot be known, and the ratio of each cell samples number of double strand cDNA CEs which is used to produce cell sample cRNA preps cannot be known to equal one. The amount of each cell sample cRNA prep produced, and the nucleotide length of the cell sample cRNA preps, are occasionally measured and known. Prior art then often compares the entire amount of each cell sample cRNA prep in the microarray assay hybridization solution. However, prior art does not determine the cRNA CE value for each compared cell sample, and therefore cannot know the assay SCR value for the microarray assay. As a consequence of the above, it cannot be known whether the compared cell sample cRNA SCR value in the assay hybridization solution, equals one or not. Thus, even though equal numbers of sample cells are used in the microarray assay, the assay SCR in the hybridization assay solution cannot be known to reflect the input compared sample cell ratio of one. In reality the assay SCR value for any particular prior art microarray assay is likely to deviate significantly from one, and can deviate by 2 to 20 fold or more. Thus, in a situation where the ratio of cells used to produce the amounts of compared cell sample T-RNA or mRNA used in the RT step is known, the actual assay SCR value for the compared cRNA preps in the microarray assay hybridization solution, cannot be known, absent further information. For such prior art microarray assays, in order to determine the assay SCR value, the CE values for each compared cell sample cRNA prep must be known, as well as the amount of each cell sample specific cRNA which is present in the assay hybridization solution. As discussed, prior art does not determine the CE for each compared cell sample cRNA prep. Therefore, the assay SCR value cannot be determined.

A variety of assay factors can affect the cDNA or cRNA CE value and therefore the SCR value for an assay. A summary of these factors follows: (i) It is known that the efficiency of isolation of RNA from cell samples is almost always significantly lower than 100%, and that the RNA isolation efficiency can be different for different cell samples, depending on the freshness of the cell sample and how the cell sample is stored, as well as other factors. (ii) It is known that the cDNA or cRNA synthesis efficiencies can vary significantly for RNAs from the same and different cell sample types. (iii) It is known that the synthesized cDNA or cRNA prep average nucleotide lengths can vary significantly for the same and different cell sample depending on the source of the cell sample and the state of degradation and/or purity of the isolated T-RNA or mRNA. These factors must be taken into consideration in order to determine the assay CE value for each compared cell sample cDNA prep or cRNA prep in a microarray gene expression comparison assay.

At present, there is no means of obtaining, or correcting for, microarray assay SCR values for cell sample cDNA or cRNA comparisons, except by the direct measurement of the cDNA or cRNA CE values for each compared cell sample, and the direct determination of the amount of cell sample cDNA or cRNA prep which is present in the assay hybridization solution. As discussed earlier, if true housekeeping genes existed for compared cell samples, then the microarray assay results from these genes could potentially be used to adjust the assay results of non-housekeeping genes for the assay SCR. As indicated, there is no evidence that such true housekeeping genes exist. Consequently, in the absence of true housekeeping genes, it is necessary to directly take into consideration these assay factors and the CE values in order to determine the assay SCR value. The earlier described methods for determining the CE values for cell sample RNA preps can also be used to determine the CE values for cDNA or cRNA preps.

For the determination of a microarray assay cell sample oligo dT primed cDNA CE value, it is necessary to know or determine a quantitative measure of the following. (i) The intact cell total mRNA CE value. (ii) The average nucleotide length of undegraded cell sample mRNA. (iii) The average nucleotide length of the cell sample cDNA prep. Here, the cell sample cDNA prep CE value is essentially equal to, (the average nucleotide length of the cell sample cDNA prep÷the average nucleotide length of the cell sample undegraded total mRNA prep)×(the CE value of the intact cell sample total mRNA).

For the determination of a microarray assay cell sample oligo dT primed cRNA CE value, it is necessary to know or determine a quantitative measure of the following. (i) The intact cell sample total mRNA CE value. (ii) The average nucleotide length of undegraded cell sample mRNA. (iii) The average nucleotide length of the cell sample cRNA prep. Here, the cell sample cRNA prep CE value is essentially equal to, (the average nucleotide length of the cell sample cRNA prep÷the average nucleotide length of the cell sample undegraded mRNA prep)×(the CE value of the intact cell sample total mRNA).

For the determination of a microarray assay CE value for cell sample T-RNA random primed cDNA, it is necessary to know or determine a quantitative measure for the CE value for the intact cell sample T-RNA. Here, the cell sample T-RNA cDNA CE value is equal to the CE value of the cell sample RNA used to produce it.

For the determination of a microarray assay CE value for cell sample isolated undegraded mRNA random primed cDNA, it is necessary to know or determine a quantitative measure for the CE value of intact cell sample total mRNA. Here, the cell sample mRNA cDNA CE value is equal to the CE value of the isolated undegraded mRNA used to produce it.

For the determination of a microarray assay CE value for cell sample isolated degraded mRNA random primed cDNA, it is necessary to know or determine a quantitative measure for the following. (i) The intact cell sample total mRNA CE value. (ii) The average nucleotide length of the intact cell sample total mRNA. (iii) The average nucleotide length of the cell sample isolated degraded mRNA. (iv) The CE value for the cell sample isolated degraded mRNA. This CE value is equal to (the average nucleotide length of the isolated degraded cell sample mRNA÷the average nucleotide length of the intact cell sample undegraded mRNA)×(the intact cell sample mRNA CE value). Here the cell sample random primed cell sample isolated degraded mRNA cDNA CE value is equal to the CE of the cell sample degraded isolated mRNA used to produce it.

Note that for microarray assays where the R and Fmole assumptions are valid for the 3′ end portions of the cell sample RNAs, and invalid for the 5′ end portions of these mRNAs, the microarray assay CDPs for each particular gene must be designed to detect the cDNA or cRNA which represents the mRNA 3′ end portion.

When a microarray cell sample comparison assay SCR value does not equal one, the assay measured DGER results must be normalized or corrected for the deviation of the assay SCR value from one, in order to obtain assay DGER results for particular gene comparisons which can be known to be biologically correct. Prior art microarray practice does not determine the assay SCR value for compared cell sample cDNA or cRNA preps in the assay hybridization solution. It is highly likely that the majority of prior art microarray assay SCR values deviate significantly from one. The reasons for this follow. (a) The almost universal use of the EA Rule in prior art microarray and non-microarray gene expression comparison practice. (b) The common occurrence of significant natural differences in the intact cell sample T-RNA and mRNA CE values which occur between cell samples of the same type, and between cell samples of different types. Such natural intact cell RNA CE value differences of 2-10 fold commonly occur between cell samples of the same type, and natural differences in the intact cell RNA CE values of 2-25 fold commonly occur between cell samples of different types. Such natural differences were discussed extensively in an earlier section. (c) The almost universal occurrence of imperfections in the process of producing cell sample isolated RNA preps and cDNA and cRNA preps, which results in the common occurrence of differences in the compared cell sample's template RNA CE values, differences in the compared cell sample's cDNA synthesis efficiencies, differences in the average nucleotide lengths of the compared cell sample's cDNA preps, differences in the amounts of cell sample cDNA produced for each compared cell sample. Differences in the cDNA synthesis efficiencies and amounts of cDNA prep produced for compared cell samples, affect the prior art microarray assay SCR values. Prior art microarray practice seldom determines the amounts of each compared cell sample cDNA prep which are compared in the hybridization solution of a microarray assay, and seldom compares equal quantities of each compared cell sample cDNA prep. This is not often the case for prior art microarray cell sample cRNA prep comparisons where equal amounts of each cell samples cRNA prep are compared. The effect of these imperfection related differences for a microarray assay cell sample cDNA or cRNA prep comparison on the assay SCR value, is independent of the effect of natural differences in the compared cell sample intact cell RNA CE values on the same assay's SCR value. In aggregate, these imperfection related differences may cancel each other out and have a minimal effect on the assay SCR value, or can interact so that their aggregate effect on the assay SCR is much greater than the effect of any one imperfection factor. In aggregate, these related imperfection differences could cause the assay SCR value to deviate from one by 1.5-5 fold or more. Current knowledge of the imperfection related factors indicates that it is reasonable to believe that aggregate effects which result in a 1.5-5 fold deviation of the assay SCR from one, are not uncommon. The overall microarray assay SCR value is influenced by both the effect of the natural differences in RNA CE values on the SCR, and the aggregate effect of the imperfection related factors on the SCR. These two influences may interact to cancel each other out, so that the deviation of the assay SCR from one is minimized. Alternatively, these two influences may interact to cause the assay SCR value to deviate from one by an amount much greater than the deviation caused by either the natural factor, or aggregate imperfection factor influence. (d) Prior art microarray practice does not determine the compared cell sample cDNA or cRNA prep CE values, and only rarely determines the cell sample cDNA synthesis efficiency or the amount of cDNA produced for an assay, and only rarely determines the amount of each compared cell sample cDNA prep which is present in the assay hybridization solution, and does not determine the number of each compared cell sample's cDNA or cRNA CEs which are present in the assay hybridization solution. Therefore, prior art microarray practice does not determine the assay SCR for cell sample cDNA and cRNA prep comparisons, and does not correct the assay measured N-DGER result for each particular gene comparison deviations of the assay SCR value from one.

For prior art microarray comparisons of cell sample cDNA or cRNA preps, assay SCR value deviations of 2-4 fold from one which are due to natural differences in the intact cell RNA CE values for compared cell samples of the same type are common. An SCR deviation from one of 10 fold can result for certain prior art microarray assay comparisons of the same cell sample type. For comparisons of different cell sample types, these natural differences can cause the assay SCR to deviate from one by 25 fold or more. The SCR deviations from one which are related to the natural differences in cell sample intact cell CE values, occur even when all aspects of the microarray assay work perfectly. In reality, the other aspects of the microarray assay rarely, if ever, work perfectly, and the assay imperfections discussed above, and others, are very common. Thus, the assay SCR values for prior art microarray assays can be affected by both the natural differences in cell sample RNA CE values, and the assay imperfections. To illustrate this, consider an assay situation where the differences in compared cell sample RNA CE values is 4 fold, and the assay imperfection related value causes a twofold deviation of the SCR from one. Here, under certain assay conditions, the assay SCR value will deviate from one by 8 fold, and the assay measured DGER value for each particular gene comparison in the assay will deviate from the true DGER (T-DGER) value for the comparison by 8 fold. Under other assay conditions, the assay SCR value will deviate from one by 2 fold.

The above discussions apply directly to the determination of cell sample cDNA prep CE values, and cell sample cDNA prep comparison SCR values for all RT-PCR gene expression assays. The methods described for the determination of cell sample CE values and SCR values for microarray assay oligo dT primed and random primed cDNA preps, can also be used to determine the cell sample CE values and SCR values for RT-PCR assay oligo dT, random and certain SG primed cDNA preps. It will be useful to further discuss the determination of RT-PCR assay CE and SCR values for cell sample cDNA preps, and to begin this with a discussion of the key requirements which are necessary for the validity of RT-PCR cell sample gene expression analysis, and cell sample gene expression analysis comparison. This discussion will be presented in a later section.

The above discussion relies on the determination of the average nucleotide lengths of degraded and undegraded RNA, cDNA, and cRNA preps. One of skill in the art will recognize that the process of determination of the proper average nucleotide length of a nucleic acid prep must take into account the nucleotide length distribution for the molecules in the nucleic acid preps, as well as a realistic model of degradation of the nucleic acid molecules in the degraded nucleic acid prep.

Simplification of Determination of Assay SCR Value for Microarray and Non-Microarray Assays. The Artificial Housekeeping Gene (AHG) Approach.

As discussed, the determination of the SCR value associated with a cell sample gene expression comparison assay can be complicated. A significant aspect of this complication involves the determination of the assay SCR value for the comparison of the cDNA or cRNA RNA equivalents. For this process, the number of cell sample mRNA, T-RNA, or other RNA cell equivalents which are compared in the assay must first be determined. Here, the number of one cell sample's RNA cell equivalents compared in the assay, is termed the RNA CE number or RCN, while the ratio of the RCN values for a cell sample comparison is termed the RCNR. The RCN and RCNR values for a cell sample LPN comparison are generally much easier to measure than the SCR value for the cDNA or cRNA produced from the RNA.

The process of determining the assay SCR value for the compared cell sample cDNAs or cRNAs, can be greatly simplified by using an exogenous standard (S) mRNA to create an artificial housekeeping gene (AHG) RNA transcript which has a known abundance in each compared cell sample mRNA or T-RNA aliquot. A general description of this AHG approach follows. (i) Determine the number of RNA CEs associated with each compared cell sample mRNA or T-RNA aliquot. (ii) Add a known mole amount of exogenous S RNA molecules of the same type to each compared cell sample mRNA or T-RNA aliquot to be compared. The mole amount of S RNA added to a cell sample RNA aliquot is termed the S RNA moles added or SM. Here, the SM will be discussed in terms of numbers of S RNA molecules added to the cell sample RNA aliquot or the number of S RNA moles added to the cell sample RNA aliquot. The ratio of, (SM for one cell sample and aliquot)÷(the SM for the other compared cell sample RNA aliquot), is termed the SM ratio, or SMR. The amount of S RNA added to each compared cell sample T-RNA or mRNA aliquot, should be an amount that ensures a strong assay signal which is far from saturation, and which minimizes signal intensity effects. For each compared cell sample RNA aliquot then, the number of S RNA copies per CE is known, and is equal to the ratio of, (the SM value for the cell sample aliquot)÷(the RCN for the same cell sample aliquot). This number of S RNA molecules per cell value is termed the SM abundance value or SMA, for the cell sample RNA aliquot. For a cell sample comparison, the ratio of the compared cell sample SMA values is termed the SMAR. Here, the SMAR can be known to equal the T-DGER for the S RNA transcript molecules in the cell sample comparison, and the exogenous S RNA transcripts qualify as valid Artificial Housekeeping Gene (AHG) RNA transcripts for the assay. Herein these S RNA transcripts, which are present in the compared cell sample RNA aliquots, are termed AHG RNA transcripts. (iii) The compared cell sample RNA aliquots are put into the assay RT step where cell sample cDNA preps are synthesized and labeled. AHG cDNA can be synthesized and labeled simultaneously. Often each compared cell sample cDNA prep is synthesized unlabeled, and then used to produce labeled cell sample cRNA. Labeled AHG cRNA is also produced by this process. (iv) The cell sample cDNA or cRNA preps are then put into the assay hybridization solution and hybridized to an array or arrays which contain CDP spots specific for the AHG cDNA or cRNA, as well as the particular gene CDP spots of interest, and other control CDP spots. After hybridization, and post-hybridization washing and processing, the RAS and RASR values associated with each AHG and particular gene comparison in the assay are determined. (v) The AHG RASR value can then be used to determine the assay SCR value which can then be used to normalize each particular gene comparison in the assay for the assay SCR value, when the assay is designed properly. Many such proper assay designs are possible. A preferred design is discussed below.

A large number of prior art microarray assays involve the comparison of Cell Sample Type 1 directly labeled LPN preps, where each compared LPN prep is labeled with a different label. A preferred improved assay design utilizing this basic prior art format is discussed in order to illustrate the use of the AHG approach for simplifying and improving the process of determining and normalizing for the assay SCR value. This preferred design involves the following. (a) Determine the intact cell T-RNA CE value for each compared cell sample. (b) Isolate T-RNA from each compared cell sample. (c) Compare isolated T-RNA aliquots in the assay. Determine the RCN value for each compared cell sample T-RNA aliquot, and the RCNR value for the cell sample T-RNA comparison. (d) Add known mole amounts of the same AHG S mRNA to each compared cell sample T-RNA aliquot. The added amounts may be equal, i.e., AHG SMR=1, or unequal, i.e., AHG SMR≠1. For each compared cell sample T-RNA aliquot the AHG SMA is known, and is equal to (SM/RCN). The cell sample comparison AHG SMAR is then equal to (SMR/RCNR) and the AHG SMAR is equivalent to the AHG T-DGER for the cell sample AHG mRNA transcript comparison. Herein, for simplicity the AHG T-DGER is termed the AHG RNA transcript ratio or AHGR. (e) Each cell sample T-RNA aliquot mixture is put into the assay RT step where SG or random priming is used to produce cDNA LPN preps for each compared cell sample. A different label is used to produce each cell sample LPN prep. The assay RT steps are designed so that the cDNA nucleotide lengths and nucleotide sequences are the same or nearly the same for each SGDS particular gene LPN or AHG S LPN comparison in the assay, and also the cell sample LPNs have LD values low enough to essentially eliminate LD effects. The production of such compared cell sample LPNs is discussed later. Here, the PAFR UNF can be ignored for normalization because cell sample T-RNAs are compared and the LPN is produced by SG or random priming. In addition, because the compared particular gene and AHG LPNs have essentially the same nucleotide lengths and sequence and TNCs, and the LD effects are negligible, then: the assay values for the UNFs MLDR, PL-HKR, PS-HKR, and PSSR are equal to one for all SGDS particular gene comparisons in the assay, and therefore these UNFs can be ignored for normalization of all SGDS particular gene and AHG comparisons in the assay, and; the PSAR UNF acts as a global NF for this assay, and has the same assay value for all SGDS particular gene and AHG comparisons in the assay. (g) The earlier discussed R and Fmole assumptions are believed by the prior art to be valid for each compared isolated cell sample T-RNA. Prior art also believes and practices that the R and Fmole assumptions are valid for each compared cell sample cDNA prep, for at least a portion of each particular gene mRNA type which is present in the isolated T-RNA. In this context then, it is quite reasonable to believe and practice that the R and Fmole assumption is also valid for the AHG cDNA which is present in each cell sample cDNA prep, and that the abundance of the AHG cDNA in a cell sample cDNA prep is known to be the same as the known AHG mRNA abundance in the cell sample T-RNA aliquot used to produce the cDNA prep. (h) A part, or all of each cell sample cDNA prep is added to a single hybridization solution, which is then incubated on an array. (i) Each such array contains replicate AHG CDP spots specific for the AHG LPN molecules, as well as cell sample particular gene CDP spots of interest, and other control CDP spots. Preferably such replicate AHG CDP spots should be made in such a way that the print tip and print plate CNF assay values are equal to one, or are not pertinent to the assay, and can be ignored for the normalization of the AHG spot results. This can be done for spotted arrays by using one tip to print all AHG spots from one AHG CDP containing well. The print tip and print plate CNFs are not pertinent for arrays where the CDP spots are synthesized on the surface. Such AHG replicate spots should be located on each such array in multiple locations, and in sufficient number to obtain a significant sampling of the spatial surface of the array. (j) Because each cell sample cDNA prep is present in the same hybridization solution, the CNF C-HKR assay value is equal to one for all AHG and particular gene comparisons, and can be ignored for normalization. After hybridization to the array under appropriate conditions, and post-hybridization washing and processing, the signal activity associated with each different label in each AHG and each other spot on the array is determined. For each replicate AHG spot, and each other spot on the array, determine the RAS value for each different label in each spot, and the RASR value for each spot. (k) At this point the pertinent UNF and CNF assay values for PAFR, MLDR, PL-HKR, PS-HKR, PSSR, C-HKR, print tip, print plate, intensity, and spatial, which are associated with an AHG spot RASR value, are equal to one or are not pertinent to the assay, and can be ignored for the normalization of each AHG spot RASR value for assay NFs. Note that the print tip and print plate CNFs are not pertinent for arrays, which are not produced by spotting. However, the assay values for the PSAR and SCR which are associated with each AHG spot RASR value, are not known, and must be determined. Here, the SCR value associated with an AHG spot can be determined, if the PSAR can be known or determined. Note that at this point the intensity CNF value associated with each AHG spot RASR value is relatively low for a properly designed AHG associated assay, and therefore the intensity CNF can be ignored during normalization of the AHG spot RASR values. However, for each particular gene comparison spot RASR value in the assay, the intensity CNF value cannot be known to be low. Therefore, for particular gene comparison spot RASR values, the associated intensity CNF value must be determined and taken into consideration during normalization. The measurement of such intensity CNFs was discussed earlier, and can be done using appropriate internal or exogenous standard replicates. (1) Each AHG spot RASR value is the result of the hybridization to one AHG spot of AHG LPN molecules associated with each cell sample LPN prep which are essentially identical except for the label. Further, the LD for each cell sample's AHG LPN molecules is known to be sufficiently low so that LD effects are essentially eliminated. Because these essentially identical AHG LPN molecules from each cell sample LPN prep hybridize to the same CDP molecules located on the surface of one AHG spot, there should be no spatial surface difference effect on the spot's RASR ratio, and the spatial CNF can be ignored for the normalization of the AHG and particular gene comparison RASR values. In such a situation, each replicate AHG spot RASR value on the array should be essentially the same. (m) The assay PSAR values associated with the replicate AHG spot and particular gene spot RASR values, should also be the same or nearly the same. Methods of determining the assay PSAR value, with or without the use of exogenous standard control molecules, are described later. (n) In this situation, the AHG spot related assay values for RCNR, AHGR, PSAR, and RASR are known by measurement and design. For the compared cell sample RNA preps, which contain the AHG mRNA, the AHG mRNA abundance ratio is the AHGR. Here, (the AHGR)=(AHG SMR)÷(RCNR). (o) The assay measured cDNA related (AHG RASR)=(AHG SMR÷RCNR) (PSAR)(SCR). This converts to (AHG RASR)=(AHGR)(PSAR)(SCR). The assay cDNA related SCR value which is associated with the AHG spot RASR can be determined from the following relationship, (SCR)=(AHG RASR)÷(AHGR×PSAR). Since the SCR is a global UNF, this same cDNA related SCR value is associated with each different SGDS, and DGDS, particular gene comparison spot RASR value in the assay. (p) In this situation, for each SGDS comparison particular gene spot RASR value in the assay, the associated assay values for PAFR, MLDR, PL-HKR, PS-HKR, PSSR, C-HKR, spatial, print tip, and print plate, are known to be equal to one, or not pertinent for the assay, and therefore can be ignored for normalization of the particular gene comparison spot RASR value. In addition, for each particular gene spot RASR value in the assay, the associated assay values for intensity, PSAR, and SCR are known by measurement and design. For the compared cell sample RNA preps which contain the particular gene mRNAs, the particular gene comparison abundance ratio is equal to the T-DGER value for the particular gene comparison in the assay. Here, (the particular gene mRNA T-DGER)=(the particular gene mRNA comparison SMR which exists for the cell sample RNA comparison)÷(cell sample comparison RCNR value). For a gene expression comparison assay, the T-DGER value associated with each particular gene mRNA comparison, is the unknown parameter, which the assay is supposed to measure. (q) Here, for an SGDS particular gene comparison in an assay, (the measured particular gene RASR value)=(particular gene SMR÷cell sample comparison RCNR) (associated intensity CNF value)(associated PSAR value)(assay SCR value). This converts to, (particular gene RASR)=(particular gene T-DGER value)(intensity CNF)(PSAR) (SCR). (r) Here, an assay measured SGDS particular gene comparison RASR value can be normalized to yield the particular gene N-DGER value which is completely normalized for all pertinent UNFs and CNFs by using the relationship (T-DGER)=(N-DGER)=(RASR)÷(intensity CNF×PSAR×SCR). Such an N-DGER value is equal to the T-DGER if the RASR is completely and validly normalized for all pertinent assay variables. Here, the assay value for PSAR is essentially the same for all SGDS particular gene and AHG comparisons in the assay. For DGDS and DGSS particular gene comparisons in this assay, the assay PSAR value is not the same for all particular gene comparisons, and in addition, it cannot be known that the assay PS-HKR UNF assay value equals one. For other assay designs the assay PSAR values associated with different SGDS particular gene comparison spots in the assay may be different, and must be determined for the normalization process. (s) The above-described assay design which is modified to compare oligo dT primed cDNA preps produced from cell sample T-RNAs or mRNAs, is also a preferred design. However, in this situation the UNF PAFR is pertinent to the cell sample particular gene comparisons in the assay, and must be taken into consideration during the normalization of the SGDS comparison particular gene spot RASR values. This is done using the relationship, (particular gene T-DGER)=(particular gene N-DGER)=(particular gene measured RASR) (PSAR×SCR×intensity CNF×PAFR). Here, the PAFR value must be determined for each particular gene comparison. Note that for such an oligo dT primed cell sample cDNA prep comparison, the PAFR is not pertinent to the AHG cDNA comparisons, and that the above-described improved, simplified method of determining the assay SCR value can be utilized. In this situation, each SGDS, DGDS, and DGSS, particular gene comparison RASR value can be normalized for the AHG determined SCR value even if the PAFR value is not known. This would result in a particular gene comparison incompletely normalized N-DGER value which is known to be validly normalized for SCR, and is therefore known to be an improved particular gene comparison N-DGER value, relative to a prior art produced particular gene comparison N-DGER value, since prior art does not determine or take into consideration during normalization, the SCR.

The above-described improved and simplified SCR determination approach can be practiced using multiple different AHG types in one assay to improve and simplify the SCR determination and normalization process, and also in the same assay using one or more different S mRNA and/or unlabeled and/or labeled S DNA types for determination of and normalization for, assay values for other assay pertinent NFs. These NFs include, but are not limited to, the UNFs MLDR, PL-HKR, PS-HKR, PSAR, PSSR, LLSR, SBNR, SSAR, and the CNFs C-HKR, spatial, print tip, print plate, intensity, scale. Many different improved assay designs are possible which utilize the improved simplified approach for determination of and normalization for the assay SCR value. Only a very small fraction of such assay designs are presented here. Such assays must be designed so that all pertinent NFs, which are associated with the AHG cDNA comparison can be known, or measured, or ignored for normalization, except for the SCR. Alternatively, such assays can be designed so that all pertinent non-global assay NF values which are associated with the AHG and particular gene comparisons in the assay, can be known or measured or ignored for normalization, and all pertinent global NFs associated with the cell sample cDNA prep comparison, are associated with the each AHG and particular gene comparison in the assay. In this situation, (the AHG RASR value which is normalized for all pertinent global NFs)=(AHGR)(the product of all assay pertinent global NF values), and this product is termed the global NF product or GNFP. The assay SCR value is included in the GNFP value. Also included in this value is any unknown assay pertinent global NF which affects both the AHG and particular gene comparison RASR values. The GNFP can be used to normalize each particular gene comparison RASR value for all assay pertinent global NF values, including the SCR. Further, (a particular gene comparison GNFP and NGNFP normalized RASR value)=(a particular gene comparison RASR value)÷(GNFP×the product of the assay pertinent non-global NF values).

Similarly, many different improved assay designs are possible which incorporate into one assay: one or more AHG or S RNA types for improving the SCR determination and to improve the determination and normalization of other pertinent NF assay values; and one or more pre-labeled or unlabeled S DNA types for improving the determination and normalization of different pertinent NF assay values. Examples of the use of such standard mRNAs or DNAs are discussed elsewhere herein.

This AHG approach for the improvement and simplification of the determination of and normalization for the assay SCR value, is also directly applicable to the comparison of cell sample cRNA preps. This is illustrated below for the comparison of differently labeled Cell Sample Type 1 directly labeled cRNA preps, produced by standard prior art methods from cell sample T-RNA, by using a preferred improved assay design. This preferred design involves the following. (a) Determine the intact cell sample T-RNA CE value for each compared cell sample. (b) Isolate and compare cell sample T-RNA preps. (c) Determine the RCN value for each compared cell sample T-RNA aliquot, and the RCNR value for the cell sample T-RNA comparison. (d) Add known mole amounts of the same S mRNA to each compared cell sample T-RNA. The amounts added should be enough to ensure a strong, but far from saturating AHG assay signal, and to further ensure that there is little or no intensity effect. The added mole amounts may be equal or unequal, i.e., the SMR=1, or SMR≠1. Here, for the compared cell sample T-RNA aliquots, the S mRNA AHGR or T-DGER value is known, and equals (SMR/RCNR). (e) Each cell sample T-RNA aliquot mixture is put into the assay RT step where T₇-oligo dT priming is used to produce an unlabeled first strand cDNA prep for each compared cell sample. Each first strand cDNA prep is then converted to double strand cDNA which contains the T₇ RNA polymerase promoter. Each double strand cell sample cDNA prep is then used to produce cell sample first round amplified label cRNA preps, where each compared cell sample cRNA LPN is associated with a different label. The cRNA synthesis step is designed so that the compared synthesized particular gene cRNA LPNs and AHG cRNA LPNs associated with the cell sample cRNA LPN comparison assay, have the same or nearly the same nucleotide lengths and nucleotide sequences, and TNCs, and also have LD values low enough to essentially eliminate LD effects. The production of such compared LPNs is discussed later. Because the compared particular gene and AHG LPNs have essentially the same nucleotide lengths and nucleotide sequences, and the LD effects are negligible, then the SGDS comparison assay values for the UNFs MLDR, PL-HKR, PS-HKR, and PSSR, are effectively equal to one for all particular gene and AHG comparisons in the assay, and therefore these UNFs can be ignored for the normalization of all SGDS particular gene and AHG comparison assay results. Also the UNF PSAR acts as a global NF and has the same assay value for all SGDS particular gene and AHG comparisons in the assay. (f) The R and Fmole assumptions are believed by the prior art to be valid for each compared isolated T-RNA. Prior art also believes and practices that the R and Fmole assumptions are valid for each compared cell sample cDNA prep produced from a cell sample T-RNA, for at least a portion of each particular gene mRNA type present in the cell sample T-RNA. Prior art also believes and practices that the R and Fmole assumptions are valid for each compared first round or second round amplified cell sample cRNA prep for at least a portion of each particular gene mRNA type present in the cell sample T-RNA. In this context then, it is quite reasonable to believe and practice that the R and Fmole assumptions are also valid: for each AHG cDNA type which is present in each cell sample cDNA prep; and for each AHG cRNA type which is present in each cell sample first or second round amplified cell sample cRNA preps; for at least a portion of each particular gene mRNA type present in the cell sample T-RNA used to produce the cDNA and cRNA preps. A consequence of this belief and practice is that the abundance value for each AHG cRNA type which is present in a cell sample first or second round amplified cell sample cRNA prep, is known to be equal to the known abundance value for the AHG mRNA type in the cell sample T-RNA aliquot used to produce the cell sample cRNA prep. (g) A part or all of each compared cell sample LPN prep is added to a single hybridization solution, which is then incubated with an array. (h) Each such array contains replicate CDP spots specific for one AHG cRNA LPN type, as well as cell sample particular gene CDP spots of interest. Preferably, such replicate AHG CDP spots should be made in such a way that the print tip and print plate CNF assay values are equal to one or are not pertinent to the assay, and can be ignored for the normalization of the AHG spot results. Such AHG replicate spots should be located on each such array in multiple locations, and in sufficient number to obtain a significant sampling of the array spatial surface. (i) Because each compared cell sample cRNA LPN prep is present in the same hybridization solution, the CNF C-HKR assay value is equal to one and can be ignored for normalization of all particular gene and AHG cRNA LPN comparisons in the assay. (j) After hybridization and post-hybridization washing and processing, the total signal activity associated with each different label in each AHG and other array spot is determined. For each replicate AHG spot, and each other spot on the array, also determine the RAS value for each label in a spot, and the RASR value for the spot.
(k) At this point the pertinent SGDS comparison UNF and CNF assay values for MLDR, PL-HKR, PS-HKR, PSSR, C-HKR, print tip, print plate, and spatial NFs, which are associated with an AHG or particular gene spot RASR value for the array, are equal to one, or are not pertinent to the assay, and can be ignored for the normalization of each AHG or particular gene RASR value. Further, for all AHG spot RASR values in the assay, the PAFR and intensity CNF can be ignored for normalization of the AHG spot RASR values. The PAFR is not pertinent for the AHG comparisons, and by design the intensity CNF assay values are essentially equal to one for the AHG comparisons. However, the PAFR is pertinent for particular gene comparison spot RASR values in the assay, and must be determined and taken into consideration during normalization. Determination of PAFR assay values and the impracticality of determining more than a very few PAFR values for an assay, was discussed earlier. In addition, the intensity CNF values associated with particular gene comparison spot RASR values in the assay, cannot be known to be low. Therefore, the particular gene comparison intensity CNFs must be determined and taken into consideration during normalization of the particular gene comparison spot RASR values. For this situation, the assay value for the UNF SCR is also not known for the cell sample cRNA LPN prep comparison. Here, the assay cRNA related SCR value can be determined if the PSAR value associated with the AHG spot RASR value is known or determined. Here, the PSAR value must be determined. (1) As described in the previous preferred AHG assay design, the spatial CNF can be ignored here for the normalization of the SGDS AHG and particular gene comparison spot RASR values. (m) The assay PSAR values associated with the SGDS comparison replicate AHG or particular gene spot RASR value, should also be the same or nearly the same. Methods of determination of assay PSAR values, with or without the use of exogenous standard or control molecules, are described later. (n) In this situation, the AHG spot associated assay values for RCNR, SMR, PSAR, and RASR, are known by measurement and design. Here, the PAFR UNF is not pertinent to the AHG comparisons, but is pertinent for the cell sample particular gene comparisons. (o) As discussed, the cRNA relatrd SCR value which is associated with the AHG spot RASR value can be determined from the relationship (SCR)=(AHG RASR)÷(AHGR×PSAR). This SCR value can then be used to normalize each SGDS, and DGDS particular gene comparison RASR value in the assay for the cRNA related SCR. (p) In this situation, for each cell sample particular gene comparison spot RASR value in the assay, the assay values for RASR, PSAR, and intensity CNF, are known by measurement and design, and the PAFR can be known by measurement, but it is impractical to directly determine the PAFR for more than a very few particular gene comparisons in a cell sample comparison assay. (q) As discussed for the previous preferred AHG design assay, in this situation, an assay measured SGDS particular gene comparison RASR value can be normalized using the relationship, (particular gene comparison T-DGER or N-DGER)=(particular gene comparison RASR value)÷(intensity CNF)(PSAR)(SCR) (PAFR).

In the above-described situation each SGDS, and DGDS particular gene comparison RASR value can be normalized for the AHG determined cRNA related SCR value, even if the PAFR and intensity CNF values are not known. This would result in particular gene cRNA LPN comparison N-DGER values which are incompletely normalized, but known to be validly normalized for the UNF SCR, and therefore improved, relative to prior art produced particular gene comparison N-DGER values, since prior art does not determine or take into consideration during normalization, the assay SCR value.

The above-described improved and simplified SCR determination approach for cell sample cRNA LPN prep comparison assays, can be practiced using multiple different AHG types in one assay to improve and simplify the SCR determination and normalization process, and in the same assay using one or more different S RNA and/or DNA S types, or one or more labeled or unlabeled S cRNA types, for the determination of and the normalization for, assay values for other assay pertinent NFs. Such NFs include, but are not limited to, the UNFs, MLDR, PL-HKR, PS-HKR, PSAR, PSSR, LLSR, SBNR, SSAR, and the CNFs C-HKR, spatial, print tip, print plate, intensity, and scale. For directly or indirectly labeled Cell Sample Type 1 or Type 2 cDNA or cRNA LPN comparisons, many different improved assay designs are possible which utilize the improved simplified AHG approach for determination of, and normalization for, the assay SCR value. Only a very small fraction is described here. Similarly, a very large number of improved assay designs are possible which combine exogenous standard methods in order to improve and simplify the determination of, and the normalization for, the SCR, and other UNFs and CNFs, for cell sample directly and indirectly labeled Type 1 and Type 2, one label and two label, cDNA and cRNA prep comparison assays. Here, only a very few are described.

The standard methods for producing amplified cRNA LPNs often produce cell sample cRNA LPN preps which contain significant amounts of cRNA which does not represent the cell sample RNA which was used to produce the cRNA LPN prep. Here, such cRNA is termed non-specific cRNA, or NS-cRNA. The presence of significant amounts of such NS-cRNA in one or more compared cell sample cRNA LPN preps can make it very difficult, if not impossible, to directly determine the assay SCR value for such a cell sample cRNA LPN comparison. The AHG approach described here makes it possible to determine the SCR even in the presence of the NS-cRNA, and greatly simplifies and improves the process of the determination of, and the normalization for, the assay SCR value associated with cell sample cRNA LPN comparison assays, as well as other assay variable normalization.

The above-described AHG approach for improving and simplifying the determination of, and the normalization for the compared cell sample cDNA prep SCR value, is also directly applicable for cell sample particular gene comparison RT-PCR assays. Such application requires the accurate determination of the PCR amplification efficiency of each compared cell sample cDNA prep.

The use of the AHG approach for improving and simplifying the determination of and the normalization for microarray and non-microarray assay SCR values, is discussed in terms of the use of exogenous standard AHG mRNAs and DNAs. However, judiciously chosen endogenous cell sample mRNAs or DNAs which represent a cell sample mRNA or a potential cell sample mRNA or other RNA produced from the cell sample DNA, can also be used as AHG mRNA or DNAs for this and other purposes.

Key Basic Requirements and Assumptions for Gene Expression Analysis and Gene Expression Comparison RT-PCR Assays.

For this discussion, it will be useful to describe a particular gene cDNA molecule, which can be detected in the PCR amplification step of an RT-PCR assay, as a cDNA amplicon equivalent molecule, or a cDNA AE molecule. Herein, the term amplicon equivalent or AE for a particular gene polynucleotide molecule indicates a polynucleotide molecule which, has a nucleotide length equal to or greater than the nucleotide length of the particular gene's primer defined double strand DNA amplicon, and which also contains the entire nucleotide sequence of one polynucleotide strand of the PCR primer defined double strand amplicon for that particular gene. Herein, an amplicon equivalent of a particular gene's RNA or mRNA is termed an RNA AE, or mRNA AE, while an AE of a particular gene's cDNA is termed a cDNA AE. Note that for a particular gene RNA or cDNA AE, the AE nucleotide length and nucleotide sequence will vary with the PCR primers used to produce the particular gene amplicon. Such nucleotide lengths can vary from 2-10 fold or more.

The earlier discussed representation or R assumption is a key assumption for the validity of the RT-PCR assay results. The reference point for this assumption is the intact sample cell, which contains undegraded T-RNA and mRNA. For cell sample isolated T-RNA, the AE R assumption is valid when at least one AE RNA molecule is present in the isolated T-RNA prep for each different particular gene expressed in the intact cell sample. For cell sample isolated mRNA, the AE R assumption is valid when at least one mRNA AE molecule is present in the isolated mRNA prep for each different particular mRNA gene expressed in the intact cell sample. For a cell sample cDNA prep, the AE R assumption is valid when at least one AE cDNA molecule is present in the cell sample cDNA prep for each different particular expressed gene mRNA which is present in the intact cell sample.

If the RNA in a cell or isolated RNA prep is very highly degraded, each individual particular expressed gene mRNA molecule present in the cell or RNA prep is fragmented into multiple fragments, which together represent a complete particular gene RNA transcript molecule. However, the nucleotide length of each RNA fragment, and any cDNA produced from such an RNA fragment, is shorter than any particular gene primer defined amplicon. In this situation if the isolated cell sample RNA and the cDNA produced from it, is too small to produce any PCR amplicons then the RT-PCR assay will be negative for all expressed genes. This is in contrast to the microarray gene expression analysis where such cDNA may be detected despite its short nucleotide length.

As discussed, it is not unusual for the RNA present in cells or isolated from cells to be degraded. When the RNA is highly degraded there may be no RNA AE molecules for any particular expressed gene present in the cell, or in the isolated cell sample RNA, or in the cDNA produced from such RNA. If the RNA is degraded, the shorter the primer defined PCR amplicon is for each particular expressed gene RNA, the more likely it will be that the AE R assumption will be valid for all, or some particular expressed gene RNA molecules, and the cDNA molecules produced from these RNA molecules. Depending on the degree of RNA degradation for a cell sample isolated RNA prep, and the amplicon primer spacing, the AE R assumption may be valid for some particular expressed gene RNAs and the cDNAs produced from them, and not valid for other expressed gene RNAs and their cDNAs. Further, the AE R assumption may be valid for all particular expressed gene RNAs present in a degraded cell sample isolated RNA prep, while the AE R assumption is valid for only some of the particular expressed gene cDNAs produced from the degraded cell sample RNA prep. This can occur because the nucleotide length of synthesized cDNA is almost always significantly shorter than the nucleotide length of the cell sample RNA template used to produce the cDNA. Prior art RT-PCR practice generally does not determine the degree of degradation of the cell sample RNA prep used to produce the cDNA for an RT-PCR assay. Further, only rarely does prior art RT-PCR practice determine the nucleotide length of the cell sample synthesized cDNA preps added to the PCR amplification solution.

The validity of the AE R assumption does not affect the qualitative interpretation of positive particular gene expression RT-PCR results obtained for a cell sample. Here, a positive result indicates that the gene is expressed in that cell sample. When the AE R assumption is invalid for one or more expressed genes, then the qualitative interpretation for a negative RT-PCR result for a gene is erroneous, and the result can be associated with a Regulation Direction Miscall, or RDM. The above discussion is applicable to oligo dT, random, primed RT-PCR assays, as well as to SG primed RT-PCR assays where the SG primers used represents all different particular gene mRNAs which are or may be present in the cell sample mRNA being analyzed.

The earlier discussed mole frequency or Fmole assumption it also a key assumption for the validity of oligo dT and random primed RT-PCR assays, as well as the SG primed RT-PCR assays where the SG primers used represent all different particular gene mRNAs which are or may be present in the cell sample mRNA being analyzed. The reference point for this requirement is again the intact sample cell, which contains undegraded T-RNA and mRNA. Using this reference point, the Fmole assumption specifies the following. (i) The AE mole frequency of occurrence, or AE Fmole, of a particular gene's RNA or mRNA transcripts in the intact cell sample, is the same as the AE Fmole of the particular gene's RNA or mRNA transcripts in the T-RNA isolated from the cell sample. Further, the AE Fmole of a particular genes cDNA transcripts which are produced from the isolated T-RNA, is the same as the AE Fmole of the particular gene's RNA transcripts which are present in the isolated cell T-RNA. Also, the AE Fmole of a particular gene's cDNA transcripts which are produced from isolated cell mRNA is the same as the AE Fmole of the particular gene's mRNA transcripts which are present in the isolated cell mRNA. (ii) When the AE Fmole of a particular gene's mRNA transcripts which are present in an intact cell, is determined on the basis of the total moles of mRNA transcripts of all kinds which are present in the cell, the AE Fmole of a particular gene's mRNA transcripts which are present in the cell, is the same or nearly the same, as the AE Fmole of the particular gene's mRNA transcripts which are present in the isolated cell T-RNA, and also the same as the AE Fmole of the particular gene's mRNA transcripts which are present in the purified mRNA isolated from the cell T-RNA. Further, in this context, the AE Fmole of a particular gene's cDNA transcripts which are produced from the mRNA present in isolated cell T-RNA, is the same as the AE Fmole of the particular gene's mRNA transcripts in the total mRNA transcript population of the isolated cell T-RNA. Also, the AE Fmole of a particular gene's cDNA transcripts which are produced from the isolated cell mRNA, is the same as the AE Fmole of the particular gene's mRNA transcripts which are present in the purified mRNA. Table 46 presents different definitions of AE Fmole for different situations. These definitions will be useful for the discussion on the determination of RT-PCR assay cDNA AE values, and SCR values.

TABLE 46
Definitions of Terms RNA AE Fmole, mRNA AE Fmole, and cDNA AE Fmole.

| Term | | Definition | |
|---|---|---|---|
| (1) | Mole frequency or AE Fmole, for a particular gene RNA which is present in a cell sample T-RNA prep. | (1) | $\frac{\begin{matrix}( Moles of RNA AE molecules for a particular \\ gene which is present in a T \text{-} RNA prep )\end{matrix}}{\begin{matrix}( Moles of particular gene RNA transcripts of all kinds \\ present in the T \text{-} RNA prep . )\end{matrix}}$ |
| (2) | Mole frequency or AE Fmole, for a particular gene mRNA which is present in a cell sample T-RNA prep. | (2) | $\frac{\begin{matrix}( Moles of m RNA AE molecules for a particular \\ gene which is present in a T \text{-} RNA prep )\end{matrix}}{\begin{matrix}( Moles of particular gene m RNA transcripts of all \\ kinds present in the T \text{-} RNA prep . )\end{matrix}}$ |
| (3) | Mole frequency or AE Fmole, for a particular gene mRNA which is present in a cell sample mRNA prep. | (3) | $\frac{\begin{matrix}( Moles of m RNA AE molecules for a particular \\ gene which are present in an mRNA prep )\end{matrix}}{\begin{matrix}( Moles of particular gene m RNA transcripts of \\ all kinds present in the mRNA prep . )\end{matrix}}$ |
| (4) | Mole frequency or AE Fmole, for a particular gene's cDNA molecules, which is present in a cell sample RNA cDNA prep. | (4) | $\frac{\begin{matrix}( Moles of RNA cDNA AE molecules for a particular \\ gene which are present in an RNA cDNA prep )\end{matrix}}{\begin{matrix}( Moles of particular gene RNA cDNA transcripts of all \\ kinds present in the cell sample RNA cDNA prep . )\end{matrix}}$ |
| (5) | Mole frequency or AE Fmole, for a particular gene's mRNA cDNA molecules, which is present in a cell sample mRNA cDNA prep. | (5) | $\frac{\begin{matrix}( Moles of mRNA cDNA AE molecules for a particular \\ gene which are present in an mRNA cDNA prep )\end{matrix}}{\begin{matrix}( Moles of particular gene mRNA cDNA transcripts of all \\ kinds present in the cell sample mRNA cDNA prep . )\end{matrix}}$ |

Prior art RT-PCR practitioners generally believe and practice that the AE Fmole assumption is valid or very nearly valid for the cDNAs used in their RT-PCR assays. This assumption may not be valid for cDNAs produced from impure or degraded cell samples. It is known that oligo dT primed cDNA produced from degraded T-RNA or mRNA, does not represent the 5′ end of some or all particular mRNAs present in the T-RNA or mRNA preps. Such cDNA represents only the 3′ end portion of the degraded mRNAs. A similar situation can occur for oligo dT primed cDNAs produced from undegraded impure T-RNA or mRNA. The impurities can cause the production of highly truncated cDNA molecules, which do not represent or are deficient in representation of the 5′ end portions of the template RNA molecules. In such a case, the Fmole assumption for the produced cDNA can be valid for the RNA 3′ end portion, and invalid for the RNA 5′ end portion. It is also known that the nucleotide length of a synthesized cDNA molecule is almost always significantly shorter than the nucleotide length of the RNA template, which produced it. One or more of the above situations can contribute to the production of oligo dT primed cDNA preps, which are short in nucleotide length, and represent only the 3′ end portion of the RNA molecules. For such cDNA preps, the AE Fmole assumption may not be valid for one or more particular expressed genes. The validity will depend on the degree of degradation of the template RNA, the degree and type of impurity present in the template RNA prep, the nucleotide length of the particular gene synthesized cDNA, the cDNA synthesis efficiency, the nucleotide length of the particular gene amplicon, and the location of the particular gene amplicon in the particular gene undegraded RNA or mRNA template. With regard to these issues, prior art practice often does not determine either the degree of degradation or impurity for the cell sample RNAs used to produce cDNA, and only rarely measures the cDNA synthesis efficiency or the nucleotide length of the synthesized cDNA.

For random primed cDNA, the AE Fmole assumption is generally believed to be valid for the RNA 3′ end portions and the 5′ end portions, even when the cDNA is produced from degraded and/or impure RNA. However, if the RNA is too impure, or too degraded, or the amount of random primer used in the cDNA synthesis too high, the cDNA nucleotide length for one or more, or all, particular gene RNAs may be smaller than the nucleotide length of the particular gene amplicon. For the cDNA of such a gene or genes, the AE Fmole assumption is invalid. Prior art RT-PCR practice only rarely determines the nucleotide length of a cDNA prep. For random primed cDNA produced from mRNA isolated from degraded T-RNA, the AE Fmole assumption is valid only for the mRNA 3′ end portions.

The validity of the cDNA AE Fmole assumption does not affect the qualitative interpretation of a positive gene expression result for a particular gene in a given cell sample, which is obtained with an RT-PCR method. A positive result means that the gene is expressed in the cell sample. However, when the AE Fmole assumption is not valid for a particular gene cDNA, the qualitative and quantitative interpretation of positive and negative results are affected. Such invalidity can result in the following. (i) An erroneous quantitative value for the number of particular gene mRNA transcripts present in a cell sample. (ii) An erroneous quantitative value for the differential gene expression ratio, which exists for a particular gene in compared cell samples. (iii) An error in the direction of gene regulation change for a particular gene, which is expressed in compared cell samples. That is a Regulation Direction Miscall (RDM) can occur. (iv) A false negative result for a particular expressed gene in a cell sample, and an RDM can be associated with the occurrence of a negative result for a particular gene comparison.

The above discussion pertains directly to the AE R and AE Fmole requirements for oligo dT and random primed assays of all kinds and may pertain to certain RT-PCR assays which use mixtures of many different SG primers. However, many, if not most, prior art RT-PCR assays, use only one or a few SG primers to produce the cDNA for only one or a few particular gene mRNAs which are present in a cell sample RNA prep.

When only one, or a few, different particular gene SG primers are used to produce cDNA from a cell sample RNA prep, only one, or a few, particular gene mRNA molecule populations will be transcribed to produce the cell sample cDNA prep. In such a situation, the AE R requirement for a particular gene cDNA prep specifies that at least one particular gene cDNA AE molecule of interest, must be present in the cDNA prep. Prior art SG primed RT-PCR assay practice believes that the AE R assumption is valid.

When only one or a few particular gene cDNAs are present in the cell sample SG primed cDNA prep, the AE Fmole parameter, is not the appropriate parameter for characterizing the particular gene's cell sample RNAs or the cDNAs produced from the cell sample RNAs. An appropriate parameter for this is the cell sample RNA or cDNA AE CE value for a particular gene. Herein, such a cell sample particular gene mRNA or cDNA AE CE value is termed a particular gene mRNA ACE value, or cDNA ACE value. The cDNA ACE value for a cell sample particular gene cDNA AE prep, is equal to the number of, or moles of, the particular gene AE mRNA transcript molecules which are present in an intact sample cell which contains only undegraded RNA. Here, by definition, the cell sample particular gene mRNA ACE value is equal to the particular gene cDNA ACE value.

In addition to the above discussed key requirements, two or more of the earlier discussed three tacit assumptions must be valid in order to obtain prior art RT-PCR assay measured particular gene RN, mRNA abundance, and N-DGER values, which are biologically accurate, and do not need to be normalized for the invalidity of these tacit assumptions. The invalidity of each of these tacit assumptions can affect the assay SCR value and the biological accuracy of RT-PCR measured particular gene RN, mRNA abundance, and N-DGER values. Earlier discussions indicated that tacit assumption one is very often invalid for prior art gene expression analysis assays of all kinds, including RT-PCR assays, and tacit assumption two is rarely valid, and tacit assumption three is seldom valid for microarray assays. Tacit assumption one will not be further discussed. Tacit assumptions two and three for RT-PCR assays are further discussed below.

It is generally believed by prior art RT-PCR practice that the use of internal or exogenous mRNA and/or DNA assay standards is necessary for obtaining meaningful RT-PCR assay measured particular gene RN, mRNA abundance, and DGER values. Prior art believes and practices that such particular gene RN, mRNA abundance, and DGER assay values, are biologically correct and do not require further normalization. The validity of this prior art belief and practice depends on the validity of each of the tacit assumptions which is pertinent to the assay. Tacit assumption one is often not valid for RT-PCR assays and the reasons for this were discussed earlier. While usually not pertinent for an RT-PCR assay, tacit assumption two is almost never valid for a microarray or RT-PCR gene expression analysis assay RIE being equal to one, and is usually invalid with regard to cell sample comparison assays. The third tacit assumption associated with prior art RT-PCR assays is complex and has been discussed in the earlier sections on “The validity of the relationship (N-DGER)=(T-DGER) when the third tacit assumption is invalid,” and “The effect of the PCR E or AE•AE values on the relationship (NASR)=(ACR) for an RT-PCR assay.” Different versions of the RT-PCR related third tacit assumption are associated with different RT-PCR assay formats, depending on whether a standard is used in the assay and the type of standard and standard strategy used for the assay. These different versions are described in the earlier section, “Key Prior Art Beliefs And Practices For Microarray And Non-Microarray Gene Expression Analysis. Three Tacit Assumptions.” It has been concluded that the prior art RT-PCR assay related third tacit assumption is rarely, and possibly never, valid for prior art RT-PCR assays. The reasons for this are summarized below. For simplicity, particular gene will be designated PG, and standard will be designated S.

Pertinent general prior art RT-PCR assay characteristics which contribute to the invalidity of the RT-PCR related third tacit assumption follow. (a) A cell sample cDNA AE•SE value, or a cell sample PG cDNA AE•SE value, or an assay associated external or internal S cDNA AE•SE value, is almost always equal to significantly less than one for an RT-PCR assay. (b) The assay cell sample cDNA AE•SE value, or PG cDNA AE•SE value, or exogenous or endogenous S cDNA AE•SE value, often varies significantly for different cell samples of the same type or different types. (c) Because of b, for cell sample PG comparisons and the associated exogenous or endogenous S comparisons, the assay AE•SER values often deviate significantly from one for a cell sample comparison assay. (d) A cell sample PG AE•AE value or PCR E value, or an assay associated exogenous or endogenous S AE•AE value or PCR E value, almost always deviates significantly from one. (e) A PG AE•AE value or PCR E value is very often significantly different for the same PG in different cell samples of the same and different types, and even for replicates of the same cell sample isolated RNA. (f) An exogenous or endogenous S AE•AE value or PCR E value is very often significantly different for different cell samples of the same and different types, and even for replicates of the same cell sample isolated RNA. (g) A PG AE•AE value or PCR E value is very often significantly different from the assay associated exogenous or endogenous S AE•AE value or PCR E value. (h) Different PG and different exogenous or endogenous S AE•AE values or PCR E values in the same PCR amplification solution are very often significantly different. (i) Because of h, for a PG comparison, or PG and S comparison, or an S comparison, the AE•AER value very often deviates significantly from one and varies significantly for different cell sample comparisons of the same and different types. (j) For an unknown cell sample it is impractical and may be impossible, to determine a PG or S AE•AE value or PCR E value with enough accuracy to rule out significant PCR E value difference effects for the assay.

A discussion of the different versions of the prior art RT-PCR related third tacit assumption follows. Here, PG specifies particular gene and S specifies standard.

For prior art RT-PCR assays which do not use a standard the third tacit assumption specifies the following. A prior art measured particular gene comparison N-DGER value can be biologically accurate only when the product of the compared cell samples (AE•SER×AE•AER), is equal to one. [Note that this third tacit assumption also applies to prior art RT-PCR assays, which use a separately generated quantitative standard reference curve to determine the particular gene comparison N-DGER values. This occurs because the external standard system is constructed for a single reference system condition, which is associated with particular S AE•SE and S AE•AE values. The compared cell samples PG AE•SE and AE•AE values may, or may not, be the same as those associated with the standard. With regard to the compared cell sample PG AE•SE and PG AE•AE values, unless it is known that the AE•SE and AE•AE values for the compared cell samples are the same as the external standard curve system, the assay situation is equivalent to not using a standard. As indicated above, it is well known that particular gene AE•SE values for compared cell sample cDNAs can be very significantly different, and can vary by 2 fold or so, depending on the type, integrity, and purity of the cell sample RNA, and the type of primer used in the RT step. In addition, it is well known that the particular gene AE•AE values of compared cell sample cDNAs often differ significantly. In addition, it appears that the assay AE•SE and AE•AE values for a cell sample cDNA are independent of each other in the assay. In this instance, in order for a prior art RT-PCR assay measured cell comparison result to be biologically accurate, the combination of four different assay values, namely Cell Sample 1 PG AE•SE and PG AE•AE, and Cell Sample 2 PG AE•SE and PG AE•AE, each of which can have a different assay value, must have just the right assay values so that the assay value for (PG AE•SER)×(PG AE•AER), equals one. This is possible but unlikely to occur often, and indicates that the third tacit assumption is likely to be invalid for most, if not virtually all, of the prior art RT-PCR assays, which do not use a standard.

In an attempt to control and normalize RT-PCR assay results for the occurrence of such significant differences in the compared particular gene cDNA AE•SE values, prior art RT-PCR practice introduced the use of internal and exogenous RNA standards for the RT step of the RT-PCR assay. Similarly, in order to control and normalize RT-PCR assay results for the occurrence of such significant assay differences in the particular gene AE•AE values, prior art RT-PCR practice introduced the use of RNA or DNA standards for the amplification step of RT-PCR or PCR assays.

For prior art RT-PCR assays, which use a DNA standard, but do not use an RNA standard, the third tacit assumption specifies the following. A prior art RT-PCR assay measured cell sample particular gene RN value can be biologically accurate only when the product of, (the particular gene AE•SE value)×(the PG/S AE•AE value), is equal to one. As indicated above, it is well known that particular gene AE•SE are almost always equal to significantly less than one, and the particular gene AE•SE assay values often vary significantly for different cell sample cDNAs. In this instance, in order for a prior art measured particular gene RN value to be biologically accurate, a combination of three different assay values, the PG AE•SE and AE•AE values and the S AE•AE value, each of which can have a different assay value, must have just the right values so that the product of, (the PG AE•SE value)×(the PG/S AE•AER value) is equal to one. This is possible but unlikely. These considerations suggest that the third tacit assumption is invalid, and the assay measured particular gene mTN values are biologically incorrect for most of these prior art RT-PCR assays.

For these prior art RT-PCR assays which use only a DNA standard in the assay, the third tacit assumption specifies that, a prior art RT-PCR measured particular gene comparison N-DGER value can be biologically correct only when the product of (the PG AE•SER assay value)×(the PG AE•AER value÷the standard AE•AER value), is equal to one. In this instance, in order for a prior art measured particular gene comparison N-DGER value to be biologically accurate, a combination of six different assay values, the Cell Sample 1 PG AE•SE and PG AE•AE values and the Cell Sample 1 associated S AE•AE value, the Cell Sample 2 PG AE•SE and PG AE•AE values and the Cell Sample 2 S A•AE value, each of which can be different, must have just the right values so that the product of, (the PG AE•SER)×(the PG AE•AER÷the S AE•AER), is equal to one. This seems highly unlikely. These considerations suggest that almost all of these prior art RT-PCR assay measured and reported particular gene N-DGER values are biologically inaccurate.

For prior art RT-PCR assays, which use an exogenous RNA standard for quantitation, the third tacit assumption specifies the following. A prior art RT-PCR measured particular gene RN value can be biologically accurate only when the product of, (the PG/S AE•SER value)×(the PG/S AE•AER value), is equal to one. As indicated, it is well known that standard AE•SE assay values are almost always equal to significantly less than one and often range from 0.1 to 0.5. Further, the standard AE•SE assay value is often very significantly different for different cell samples. Here, a total of four different assay values are associated with this assay, the PG AE•SE and AE•AE values and the S AE•SE and AE•AE values. In the assay, each of these 4 assay values can have a significantly different assay value. In order for a prior art measured particular gene RN value to be biologically correct, the combination of four separate assay values must be associated with just the right assay values so that the value of the product of, (the PG/S AE•SER value)×(the PG/S AE•AER value), is equal to one. This is unlikely but not impossible. These considerations suggest that the third tacit assumption is invalid, and the assay measured particular gene mTN values are biologically incorrect, for most of these prior art RT-PCR assays.

For prior art RT-PCR assays which use an internal RNA standard such as a housekeeping gene mRNA, or an exogenous RNA standard, for cell sample particular gene comparisons, the third tacit assumptions specifies the following. A prior art RT-PCR measured particular gene comparison N-DGER value can be biologically accurate only when, (the PG AE•SER value×the PG AE•AER value)÷(the S AE•SER value×the S AE•AER value), is equal to one. Here, a total of eight different AE•SE and AE•AE assay values are associated with this assay, the Cell Sample 1 values for PG AE•SE and S AE•SE, PG AE•AE, S AE•AE, and the Cell Sample 2 values for PG AE•SE, S AE•SE, PG AE•AE, and S AE•AE. In the assay, each of these different assay values can be significantly different. In order for a prior art measured particular gene comparison N-DGER value to be biologically accurate, the combination of eight separate assay values must have just the right assay values so that the value of, (the PG AE•SER value×PG AE•AER value)÷(the S AE•SER value×the S AE•AER value), is equal to one. This is highly unlikely. These considerations suggest that the third tacit assumption is invalid, and the particular gene comparison N-DGER values are not biologically accurate for most, if not almost all, of these prior art RT-PCR assays.

Prior art relative RT-PCR assays often use a housekeeping gene (HG) internal standard for the normalization of particular gene N-DGER values for differences in the PG AE•SE and PG AE•AE values of the compared cell sample cDNAs. This method is based on the assumptions that housekeeping genes exist, and can be identified, and that for each cell sample cDNA compared, the ratio of the PG/HG AE•SE values is the same, and that for each cell sample cDNA compared, the ratio of the PG/HG AE•AE values is also the same. Prior art RT-PCR practice acknowledges that generally applicable housekeeping genes have not been identified, but certain prior art RT-PCR practitioners believe and practice that housekeeping genes have been identified which can be used for restricted situations. However, such prior art housekeeping genes were identified using prior art methods which do not take the SCR and other prior art pertinent assay UNFs into consideration, and therefore cannot be known to be valid housekeeping genes, even for the restricted situation. In addition, as discussed above, while it is likely that the assay PG/HG AE•SER value will equal one for each compared cell sample cDNA prep, it is not likely that the PG AE•AE and HG AE•AE values for a compared cell sample cDNA will be the same, or that the PG AE•AE and HG AE•AE values for the different compared cell samples will be the same.

Prior art RT-PCR practice rarely determines and normalizes for differences in assay particular gene or standard AE•SE or AE•AE assay values. When such determinations are done, the determination is done for one particular gene, one standard, and one cell sample. Prior art then assumes that the PG and S AE•SE and AE•AE values are similar for other cell sample cDNA preps, and can be validly used for other samples. As discussed, this prior art assumption is invalid for RT-PCR assays in general. Differences in the compared cell sample's and standards AE•SE and AE•AE values have a directly proportional effect on the magnitude of deviation of the particular gene mTN or measured DGER value from biological accuracy. However, very small differences in the PCR amplification efficiency value E, results in large differences in cell sample particular gene and standard assay AE•AE values. Prior art RT-PCR assay E values for particular genes and standards generally range from 0.7 to 0.9. For an RT-PCR assay, which uses 30 cycles of amplification, a 0.7-0.9 range of E values translates into a 26 fold difference in the assay value for AE•AE. Very small differences in the assay values for a particular gene in one cell sample cDNA prep, and a particular gene in a compared cell sample cDNA prep, translate into significant differences in the AE•AE values for the compared cell samples cDNAs, and a significant deviation of the assay particular gene AE•AER value from one. This can be illustrated by considering an RT-PCR assay with the following characteristics. (a) The assay uses 30 amplification cycles. (b) E=0.8 for one cell sample particular gene cDNA. (c) E=0.84, for a second compared cell sample same particular gene cDNA. (d) E=0.76 for a third compared cell sample same particular gene cDNA. (e) E=0.8 for the standard. (f) The particular gene mRNA abundance is one copy per cell for all cell samples, and the particular gene T-DGER equals one for all cell sample comparisons. Here, the cell sample two and three E values differ by only 5 percent from the E of cell sample one. Such an E difference translates into a first sample/second sample PG AE•AER value of about 0.5, a first sample/third sample AE•AER value of about 2, and a second sample/third sample AE•AER value of about 4. For an RT-PCR particular gene comparison, a PG AE•AER value of 2 for the cell sample one/cell sample three comparison will cause the cell sample one assay measured particular gene RN value to equal twice the cell sample three particular gene RN value, and thereby cause the measured particular gene DGER value to deviate from biological accuracy by 2 fold. Here then, a 5 percent difference in the E values of compared cell samples cDNAs, causes: (i) A two fold difference in the compared PG AE•AE values; (ii) An assay particular gene AE•AER value equal to two; (iii) A two fold deviation of the assay measured particular gene N-DGER value from biological accuracy.

Prior art practice generally claims an accuracy of measurement for particular gene RN values, and particular gene N-DGER values, of ±1.2 to 2 fold. Certain prior art RT-PCR assays claim an accuracy of measurement for particular gene N-DGER values of ±1.2 fold. In this context, small differences in the E values for the assay cell sample cDNAs and standard cDNAs, result in large differences in the assay AE•AE values, which then cause significant deviations of the measured particular gene assay measured RN and N-DGER values from biological accuracy. Further, these deviations from biological accuracy can have a magnitude which is near or greater than the assay accuracy, even when the difference or difference in the particular gene or standard assay value for E is quite small. For 30 cycle RT-PCR assays, a change in the value of E of 2.5 to 10 percent can cause an assay measured particular gene RN or N-DGER value to deviate from biological accuracy by 1.4 to about 3.7 fold. Note, that other non-E assay factors can cause such E associated deviations to be even larger, or smaller. Prior art RT-PCR practice only rarely determines and normalizes for assay particular gene or standard E values, and as discussed earlier, it is not unusual for such values to be associated with a measurement accuracy of ±10% or more. Note that the maximum theoretical assay value for E is one, and the measured assay values for E generally equal 0.7 to 0.9. Absent knowledge of the assay particular gene and standard E and AE•AE values, which is not provided by the prior art, it cannot be known that the prior art RT-PCR measured particular gene RN, mRNA abundance, and N-DGER values are biologically accurate or not, within the stated accuracy of the assay. The measurement of the particular gene and/or standard E or AE•AE values is a daunting task. Given the large number of assay factors which can affect the particular gene or standard E and AE•AE values, it may be necessary to measure the particular gene and standard E and/or AE•AE assay values for each cell sample cDNA associated with the assay, and then to normalize for differences. Determination of even a single E or AE•AE particular gene or standard assay value is a complex task. The process of determining the effect of E or AE•AE differences on the measured particular gene RN, mRNA abundance, and N-DGER values, is greatly complicated by the necessity to determine the assay values for the particular gene and standard AE•SE values. Such values can magnify, or diminish, the effect of assay differences in particular gene and/or standard E and AE•AE assay values. Prior art only rarely determines the particular gene or standard AE•SE values for an assay. Adding to the complication of determining and normalizing for differences in particular gene and/or standard E and AE•AE values, is that it appears that such differences are associated with both non-global and global assay variable factors, and relatively little information is available concerning the differences, and the assay factors which cause them.

RT-PCR assays are often used to corroborate microarray assay measured particular gene N-DGER values. Both RT-PCR and microarray assays involve an RT step, and both rely on being able to accurately measure the relative concentrations of a particular gene cDNA in compared cell sample cDNA preps. However, microarray assay measured particular gene N-DGER values are not associated with particular gene and standard E and AE•AE values. Microarrays are associated with other assay variables which are not associated with RT-PCR assays, and like the E and AE•AE values, prior art microarray practice only rarely, if ever, determines and normalizes for these unconsidered assay variables. Such a situation indicates that the prior art use of RT-PCR assay measured particular gene N-DGER values to corroborate the quantitative magnitude of the microarray assay measured particular gene N-DGER value, and the direction of gene regulation change implied by the microarray N-DGER value, is problematic at best.

The third tacit assumption for microarray assays involves only the cell sample cDNA synthesis efficiency, and is associated only with the unconsidered assay variable global NF, the SCR. The RT-PCR versions of the third tacit assumption involve both the AE•SE and the AE•AE values. The RT-PCR assay AE•SE value is associated with the assay global NF, the SCR. Here, the assay AE•AE value does not affect the assay SCR value, and is independent of the SCR, and is a non-global assay variable.

A small fraction of prior art RT-PCR gene expression analysis assays is designed to determine the number of particular gene mRNA transcripts per cell for a cell sample, i.e., designed to determine the particular gene mRNA abundance value for the cell sample. Prior art generally believes and practices that such a particular gene mRNA abundance value is biologically accurate for the cell sample. The validity of this belief depends on the validity of tacit assumptions two and three for the assay. This will be discussed below in terms of the effect of the validity of the second tacit assumption on the biological accuracy of such an RT-PCR measured particular gene abundance value. For simplicity, this discussion will assume that the third tacit assumption is valid for the RT-PCR assay. In this situation, when the second tacit assumption is invalid for the particular gene the RT-PCR measured value for the particular gene mRNA abundance is not biologically correct, and is therefore erroneous. Prior art RT-PCR practice does not determine the validity of tacit assumption two and take it into consideration during normalization. It is known that the efficiency of RNA isolation can vary significantly for different cell samples of the same type, or for different cell sample types. In addition, prior art does not determine the amount of T-RNA or mRNA per cell for intact cells. As a result, the cell sample T-RNA or mRNA CE values used by the prior art to determine the number of RNA CEs present in the RT step of the assay are inaccurate, and the resulting particular gene mRNA abundance values for the cell sample particular gene are biologically inaccurate, even when the measured particular gene mRNA transcript RN value used to determine the abundance value, is biologically accurate. Under these assay conditions, the RT-PCR assay measured ratio of the particular gene mRNA abundance values is biologically inaccurate, unless the RNA isolation efficiency for each cell sample RNA is the same. Absent knowledge of the compared cell sample RNA isolation efficiencies, it cannot be known whether a prior art measured particular gene mRNA abundance value or compared mRNA abundance value ratio, is biologically accurate or not.

Since prior art RT-PCR gene expression analysis assays almost always involve an SGDS comparison of particular gene mRNA transcripts, the above discussion has emphasized such assay analyzes. However, the discussion applies directly to all SGDS, DGDS, and DGSS comparisons of viral, prokaryotic, eukaryotic, and standard RNA transcripts of all kinds. This includes all types of rRNA, tRNA, mRNA, siRNA, miRNA, snoRNA, antisense RNA, and other known or unknown RNAs.

Determination of RT-PCR Assay CE Values for Oligo dT Primed or Random Primed Cell Sample cDNA Preps.

For an RT-PCR assay which utilizes oligo dT primed cell sample cDNA, in order to determine the CE value for the cell sample cDNA prep used in the assay, it is necessary to know or determine the following. (i) The intact cell total mRNA CE value. (ii) The average nucleotide length of the cell sample undegraded total mRNA. (iii) The average nucleotide length of the cell sample cDNA Prep. Here, the cell sample cDNA prep CE value is equal to, [the average nucleotide length of the cell sample cDNA prep÷the average nucleotide length of the cell sample undegraded total mRNA]×[the CE value for the intact cell sample total mRNA]. This applies to oligo dT primed cDNA preps produced from degraded or undegraded cell sample T-RNA or isolated mRNA.

For an RT-PCR assay which utilizes an SG primer mixture which represents all different particular gene mRNAs which are or may be present in the analyzed cell sample RNA prep, the requirements for determining the CE value for the cell sample cDNA prep are the same as those for dT primed cDNA preps when all of the particular gene SG primers are targeted to the extreme 3′ end portion of the mRNA.

For an RT-PCR assay which utilizes random primed cell sample cDNA produced from isolated T-RNA, in order to determine or know the CE value of the cell sample cDNA prep, the CE value of the isolated cell sample T-RNA prep must be known. Here, the CE value for the cell sample random primed cDNA is equal to the CE value of the cell sample T-RNA prep, which is used to produce it. This applies to such cDNA preps produced from degraded or undegraded cell sample isolated T-RNA.

For an RT-PCR assay which utilizes random primed cDNA produced from isolated cell sample mRNA, in order to determine or know the CE value of the cell sample cDNA prep, the CE value for the cell sample isolated mRNA prep must be known. Here, the CE value for the random primed cDNA is equal to the CE value of the isolated cell sample mRNA prep which is used to produce it. This applies to such cDNA preps produced from degraded or undegraded isolated cell sample mRNA.

Determination of RT-PCR Assay SCR Values for Compared Cell Sample Oligo dT and Random Primed cDNA Preps.

In order to determine the SCR value for an RT-PCR assay comparison of different cell sample oligo dT or random primed cDNA preps, it is necessary to know or determine the following. (i) The amount of each compared cell sample cDNA prep, which is present in the assay PCR amplification solution. (ii) The assay CE value for each compared cell sample cDNA prep. (iii) The number of each compared cell sample's CEs which is present in the assay PCR assay amplification solution. For each compared cell sample cDNA, the number of CEs present in the assay PCR solution is equal to, (the amount of cell sample cDNA present in the PCR amplification solution)÷(the CE value for the cell sample cDNA prep). The RT-PCR assay SCR value is then equal to the ratio for the assay PCR amplification solutions of, (the number of one cell sample's cDNA CEs present in the PCR amplification solution)÷(the number of the other compared cell sample cDNA CEs present in the PCR amplification solution).

Table 42 presents a summary of prior art RT-PCR assay practices with regard to the determination of CE values for cell sample cDNA preps, and SCR values for the RT-PCR comparison of cell sample oligo dT or random primed cDNA preps. For most prior art RT-PCR assays, very small amounts of cell sample RNA is used in the RT step and, therefore, very little cDNA is produced. Such amounts are often not quantifiable unless labeled with radioactivity. As discussed earlier, it is known that the cDNA synthesis efficiency can vary greatly for the same and different cell samples. If the amount of cDNA produced in the RT step is not known, then the amount of cell sample cDNA, which is present in the assay PCR amplification step, cannot be determined or known. In addition, in this situation it is often not possible to directly determine the average nucleotide length of the cell sample cDNA prep unless the cDNA is labeled with radioactivity. As described later, it is possible to indirectly determine the average nucleotide length of a cDNA prep with the use of LPN molecules which are complementary to the cDNA.

As indicated in an earlier discussion, it is essential to know the SCR value for a microarray or non-microarray gene expression comparison measured assay in order to correctly normalize the particular gene N-DGER values when the assay SCR value is not equal to one. As an example, an RT-PCR gene expression comparison assay, which compares equal amounts of cell sample cDNA, can have an assay SCR which deviates from one by a large factor. If the assay SCR value for such an assay is not determined, and used to correct the RT-PCR measured DGER result, and all other aspects of the RT-PCR assay work perfectly, the measured RT-PCR assay N-DGER result can deviate from biological accuracy by a large factor as a result of the invalidity of the first tacit assumption. When the second and third tacit assumptions are also invalid, the deviation can be potentially 50-75 fold or more. Prior art RT-PCR assay practice does not determine the validity of tacit assumptions one, two, and three, or the assay SCR value. Because of this it cannot be known whether a particular RT-PCR assay SCR value deviates significantly from one or not. Therefore, it cannot be known whether the assay SCR value will cause the assay measured N-DGER result to deviate significantly from the T-DGER value or not. As a consequence, prior art RT-PCR assay measured particular gene comparison DGER results cannot be known to be biologically correct or incorrect, and are therefore uninterpretable with regard to biological correctness.

Absent knowledge of the assay SCR value, prior art RT-PCR assay measured N-DGER results cannot be known to be biologically correct, even for the following prior art RT-PCR assay approaches. Approach One. Known equal amounts of compared cell sample T-RNA or mRNA from cell samples which are believed to have the same known intact cell T-RNA or mRNA CE value are used in the RT step of the assay. Approach Two. An amount of each compared cell sample T-RNA or mRNA which represents a known number of cells for each cell sample, is used in the RT step of the assay. Here it is assumed, as does the prior art, that the PCR amplification step accurately measures the absolute or relative amounts of a particular gene cDNA which is present in each cell samples cDNA preps.

For Approach One, when the EA Rule is practiced for the comparison of cell sample T-RNA or mRNA preps which have the same intact cell T-RNA or mRNA CE values, and for Approach Two, where the intact cell T-RNA or mRNA CE values may not be the same for the compared cell sample T-RNA or mRNA preps, the amount of each cell sample T-RNA or mRNA used in the RT step represents a known number of cells for each compared cell sample. From these cell sample RNAs each cell sample cDNA prep is produced, and the entirety of each cell samples cDNA prep, is then put in the assay PCR amplification solution. Here, it cannot be known that the assay SCR value for the compared cDNA preps is equal to, (the number of RNA CEs for one cell sample which is used in the assay RT step)÷(the number of CEs for the other compared cell sample which is used in the assay RT step).

Certain prior art RT-PCR gene expression analysis and gene expression comparison assays, use an amount of each compared cell sample T-RNA or mRNA in the RT step which is claimed to represent the same known number of sample cells for each compared cell sample. These prior art assays believe and practice that biologically accurate values for particular gene mRNA transcript number and mRNA abundance, which are associated with each compared cell sample, as well as biologically accurate particular gene N-DGER values, are obtained from such prior art assays. These assays are versions of the above-described Approach One assays. Such assays were discussed in the earlier section on the validity of the second tacit assumption. These prior art Approach One assays utilize what is regarded as a known measured and accurate value for the amount of T-RNA or mRNA per cell for each compared cell sample, and then use this value to determine the number of cells for each cell sample which are represented by the amount of each cell samples T-RNA or mRNA which is used in the assay RT step. The second prior art approach is to isolate the T-RNA or mRNA from a known number of cells for each cell sample, and then use all, or the same fraction, of the isolated T-RNA or mRNA from each cell sample in the assay RT step. For both approaches the number of cells in the assay RT step which is represented by the amount of cell RNA present in the RT step, is believed to be known for each compared cell sample. It will be useful to discuss prior art beliefs and practices with regard to Approach One and Two assays. Prior art practices with regard to RT-PCR assays in general are presented in Tables 42 and 43.

For Approach One, a prior art measured value for the amount of T-RNA or mRNA per cell is used to determine the number of cells or cell equivalents for each compared cell sample which is present in the assay RT step. Prior art RT-PCR, microarray, and other non-microarray gene expression analysis and gene expression comparison analysis practitioners, believe and practice the first tacit assumption, i.e., the compared cell samples have the same or essentially the same intact cell CE values for T-RNA or mRNA. For almost all prior art RT-PCR, microarray, and other non-microarray gene expression analysis and gene expression comparison analysis assays, the intact cell T-RNA or mRNA CE value for a cell sample or for either compared cell samples has not been determined, or known. Prior art practice tacitly assumes that the intact cell T-RNA and mRNA CE values for each compared cell sample in an assay is the same or, essentially the same.

Further, for all or almost all of the rare prior art RT-PCR and other gene comparison assays where the amount of T-RNA per cell or mRNA per cell was determined for a cell sample, the determination was done for only one of the compared cell samples, and it was tacitly assumed that each of the compared cell samples in the assay had essentially the same, or nearly the same amount of T-RNA or mRNA per sample cell. Generally such a measurement is done to provide a basis for determining the abundance values for particular gene mRNAs in a cell sample, and prior art believes and practices that such a measurement allows the accurate determination of mRNA abundance values for particular genes in each compared cell sample. Almost all prior art RT-PCR, microarray, and other non-microarray gene expression analysis and gene expression comparison analysis assays practice tacit Assumption One, even though knowledge of common significant differences in the intact cell T-RNA and mRNA CE values has been known for forty years or more. Intact cell T-RNA or mRNA CE values for different cell samples of the same type often differ by 2-10 fold, while such values commonly differ by 2-25 fold for different cell sample types from the same organism. Such differences were extensively discussed earlier.

For RT-PCR, microarray, or other non-microarray prior art Approach One assays, as well as other prior art gene expression analysis and gene expression comparison analysis assays, tacit assumption one must be valid in order to know the number of RNA cell equivalents used in the RT step of the assay, and to know that the same number of RNA CEs for each compared cell sample, is used in the RT step. If tacit Assumption One is invalid, then absent further information which is not determined by the prior art, the assay measured abundance values for particular genes in a cell sample, and the assay measured DGER values for particular genes in a cell sample comparison, cannot be known to be biologically accurate, and are uninterpretable.

Prior art also believes and practices that when the amount of T-RNA per cell or mRNA per cell for a cell sample is determined by quantitating the amount of T-RNA and/or mRNA isolated from a known number of sample cells, and then determining the amount of T-RNA and/or mRNA per sample cell, the value for the amount of T-RNA per cell or mRNA per cell is essentially equal to the cell sample's intact cell CE value for T-RNA or mRNA. In order for this to be true, the second tacit assumption must be valid for the assay, i.e., the cell sample RNA isolation efficiency must equal one. Almost all, if not all prior art RT-PCR, microarray, and other non-microarray assays which involve the above-described first or second approaches, tacitly assume this, even though it is known that the efficiency of isolation of T-RNA and mRNA from intact cells of different cell samples of the same type, and different cell sample types, can vary significantly. This tacit assumption must be valid for both Approach One and Two assays, and other prior art gene expression analysis and gene expression comparison analysis assays, in order for the prior are practitioner to know that the same number of RNA cell equivalents is used in the RT step of the assay. If the assumption is invalid, then the assay measured abundance values for particular genes in each compared cell sample, and the assay measured DGER results for particular genes cannot be known to be biologically accurate, and therefore are uninterpretable.

Prior art RT-PCR and other prior art gene expression analysis assays also believe and practice the third tacit assumption, i.e., the number of cell sample RNA cell equivalents or CEs used in the assay RT step, is the same or essentially the same as the number of cell sample cDNA CEs which is produced from the cell sample RNA in the RT step. Almost all, if not all prior art RT-PCR and other gene expression analysis assays which involve the above-described Approach One or Approach Two, tacitly assumes this third assumption, even though it is known that imperfections in the RT step almost always results in an amount of produced cell sample cDNA which is significantly less than the amount of cell sample RNA present in the RT step. In addition, the effect of the RT step imperfections on the fraction of cell sample RNA produced as cDNA, can vary significantly for compared cell sample RT steps. This third tacit assumption must be valid in order to determine the number of cell sample cDNA CEs which are produced in the RT step of the assay, and which are present in the assay PCR amplification solution or the microarray hybridization solution. If this third assumption is invalid, then the prior art assay measured mRNA transcript number values and mRNA abundance values for particular genes in a cell sample, and any DGER values derived from these particular gene mRNA transcript number values or mRNA abundance values, cannot be known to be biologically accurate, and therefore are uninterpretable.

Specific prior art examples, which utilize the above-described first and second approaches, are discussed in a later section.

Determination of the Number of Particular Gene ACE's and the SCR for an SG Primed RT-PCR Assay.

One purpose of defining and determining the cDNA cell equivalent is to facilitate the quantitation and determination of the assay SCR value for a gene expression comparison assay. As discussed, for cell sample cDNA preps produced with oligo dT and random primers it is possible to define and quantitatively determine in a practical way, CE values for the intact sample cell RNA, the isolated cell sample RNA prep, the cell sample cDNA prep, and the cell sample cRNA prep, in terms of the bulk properties of the total cell sample T-RNA or mRNA, the cell sample cDNA prep, or the cell sample cRNA prep. This cannot be done for a cell sample cDNA prep which is produced using only one, or a few, particular gene SG primers. The reasons for this are discussed below.

A large fraction of all prior art RT-PCR assays use only one or a few SG primers to produce the cell sample cDNA which is analyzed. Such a cell sample cDNA prep consists of only one or a few particular gene cDNAs of interest. Because of this, the cell sample cDNA CE must be defined in terms of a particular gene's cDNA molecules in order to determine the number of cell sample particular gene cDNA CEs which are present in the assay PCR amplification solution of the assay, and the assay SCR value. As defined earlier, an amplicon cell equivalent or, ACE value, for a particular gene mRNA transcript molecule population in an intact sample cell, is equal to the number of or moles of, or average number of or moles of, the particular gene mRNA transcripts per cell. An ACE value for a particular mRNA transcript in a cell is therefore, equal to the particular gene mRNA abundance value for the cell. For a cell sample particular gene cDNA prep, the particular gene cDNA ACE value, is then equal to the particular gene mRNA transcript ACE value for the cell sample RNA of interest. Prior art SG primed RT-PCR gene expression analysis and comparison assays are discussed in more detail below. For simplicity this discussion will be in terms of the cDNA produced by one SG primer which is specific for a cell sample particular gene mRNA of interest. In addition, it will be assumed, as does the prior art, that the AE R assumption is valid for the cell sample SG primed cDNA prep. It will also be assumed, as does the prior art, that the AE Fmole assumption is valid for the cell sample isolated T-RNA or mRNA used in the RT step to produce the cDNA used in the assay. Further, the prior art SG primed RT-PCR assay will involve the following. (i) Isolate cell sample T-RNA or mRNA. (ii) Use a known amount of cell sample RNA in the RT step. This RT step may or may not contain known amounts of one or more exogenous standard mRNA transcripts, and the appropriate SG primers for them, or SG primers for one or more particular endogenous housekeeping gene standards. (iii) Produce the cell sample cDNA prep. (iv) Put the entirety of the produced cell sample cDNA prep into the assay PCR assay amplification solution. (v) Amplify and determine a measure of the number of particular gene and standard amplicons, which have been produced, and use this information to determine the assay measured particular gene RN value. (vi) For cell sample particular gene comparisons, a known equal amount of each compared cell sample RNA prep is added to the assay RT step. (vii) For a cell sample comparison the particular gene measured N-DGER value is equal to the ratio of the assay measured particular gene RN values, and the particular gene T-DGER value equals one. (viii) For a cell sample the number of RNA CEs which are present in the RT step is termed the RNA cell equivalent number, or RCN. For a cell sample comparison, the compared cell sample RCN ratio is termed the RCNR. (ix) For a cell sample comparison the assay SCR value is equal to the ratio of, (the number of SG produced particular gene cDNA ACEs for one cell sample)÷(the number of particular gene SG produced cDNA ACEs for a compared cell sample). (x) For a cell sample SG primed cDNA, the synthesis efficiency of the particular gene cDNA from the particular gene mRNA transcripts present in the RT step is termed the cDNA AE•SE, and for a cell comparison RT-PCR assay the ratio of the compared cell sample's cDNA SEs is termed the cDNA AE•SER. Note that the cDNA synthesis efficiency is defined in terms of the ratio of, (the number of particular gene cDNA AE molecules which are produced in the RT step)÷(the number of particular gene mRNA AE transcript molecules present in the RT step).

For an SG primed RT-PCR assay it is possible to determine the number of cell sample RNA CEs which are used in the assay RT step from the bulk properties of the cell sample RNA. Such a determination was described earlier. For such an assay the number of cell sample RNA CEs present in the RT step, is equal to the number of particular gene mRNA transcript ACEs which are present in the assay RT step. Clearly when the assay RT step works perfectly and the particular gene cDNA synthesis efficiency or AE•SE, is equal to one, then the number of cell sample cDNA ACEs produced in the RT step, and present in the assay PCR amplification step, is equal to the known number of cell sample RNA CEs which are used in the RT step. This will occur only if the particular gene cDNA AE•SE value equals one. It is well known that a particular gene cDNA AE•SE value only rarely if ever, equals one, and generally equals from 0.1 to 0.5. As a result, the number of cell sample particular gene cDNA ACEs produced in the RT step is virtually always very significantly smaller than the number of cell sample RNA CEs, or cell sample particular gene mRNA transcript ACEs, present in the assay RT step. In addition, it is not possible to directly determine the number of cell sample particular gene cDNA ACEs produced in the RT step, or present in the assay PCR amplification step. This occurs for the following reasons. (a) The SG primed cell sample cDNA prep consists of only the particular gene cDNA of interest and in essence has no bulk properties. (b) While rarely done by the prior art, the amount of particular gene cDNA produced and the number of particular gene cDNA AE molecules produced in the RT step can be determined. (c) In order to determine the number of cell sample particular gene cDNA ACEs produced in the RT step it is necessary to know the cell sample particular gene mRNA ACE value, which is equal to the particular gene mRNA abundance value for the cell sample. (d) The particular gene mRNA abundance value is the assay unknown, and therefore it is not possible to directly determine the number of cell sample particular gene cDNA ACEs produced in the RT step, or present in the PCR amplification solution.

For prior art SG primed RT-PCR assays, only when the particular gene cDNA AE•SE value is known to equal one, can it be known that the number of cell sample particular gene cDNA ACEs produced in the RT step is equal to the number of cell sample RNA CEs used in the assay RT step. Here, the number of cell sample RNA CEs present in the RT step of the assay is termed the RNA CE number or RCN, while the number of cDNA ACEs which are produced in the RT step is termed the cDNA ACE number or CCN. Here then, when the assay cDNA AE•SE value equals one, in the RT step of the assay (the cell sample RCN value)=(the particular gene CCN value). When the assay value for the particular gene cDNA AE•SE is not equal to one, then for the assay RT step, (the cell sample RCN value)=(the particular gene CCN value÷the particular gene cDNA AE•SE value). As discussed, the prior art cell sample particular gene cDNA AE•SE value is almost always equal to significantly less than one and is usually 0.1 to 0.5. Further, prior art SG primed RT-PCR assay practice rarely if ever, determines the cDNA AE•SE and RCN assay values.

The relationship, (cell sample RCN)=(particular gene CCN÷particular gene cDNA AE•SE), can be used to determine the particular gene CCN, if the cell sample RCN value, which can be directly determined, and the particular gene cDNA AE•SE value, are known for the cell sample SG primed RT-PCR assay. The particular gene cDNA AE•SE value cannot be directly determined, but can be determined indirectly. Such an indirect determination involves the use of an exogenous mRNA standard in the RT mix, and relies on the prior art belief and practice that in the same cell sample RT reaction solution the AE•SE value is the same for all cell sample and standard mRNAs present. This can be done for a cell sample T-RNA or mRNA of interest and requires knowing very accurately the E value for the standard cDNA AE molecules in the assay. It is here assumed that such accurate E measurements can be produced using prior art methods. Such determination of a cell sample particular gene AE•SE value involves the following. (a) To an RT step containing a known amount of cell sample RNA which is associated with a known RCN value, add a known number of S mRNA molecules and an appropriate SG primer for the S mRNA. Here, the number of a particular gene mRNA transcript molecules present in the cell sample RNA is termed the PG RN value, while the known number of S mRNA transcript molecules present in the same cell sample RNA is termed the S RN value. (b) Produce the cell sample particular gene cDNA AE molecules and the S cDNA AE molecules in the RT step and put the entirety of the cDNA produced into the PCR amplification step solution. (c) Amplify the particular gene and S cDNA amplicons for a known number of cycles. (d) Determine the number of particular gene amplicons PGN, and the number of standard amplicons SN produced in the amplification step. Here, the number of PG and S cDNA AE molecules put into the amplification solution is termed the PGo and So. (e) The PGo and So values for the assay can be determined using the known S and PG E values and the measured PGN and SN values. This is done using the well known relationship PGo=(PGn)÷(1+PG E)^{N} or So=(SN)÷(1+S E)^{N}, where N is the number of amplification cycles. The S AE•SE value for the assay is then equal to (S AE•SE)=(measured So value)÷(known S RN Value). (f) Prior art believes and practices that the R and Fmole assumptions are valid for different particular genes and standard cDNAs in a cell sample cDNA prep. If the R and Fmole assumptions are valid for both particular gene and S cDNAs, then for the cell sample cDNA synthesis step, the AE•SE values for the different particular gene and S cDNAs which are produced are the same. Therefore, for this RT reaction the (PG AE•SE)=(S AE•SE). (g) Determine the PG AE•SE value for each compared cell sample and the PG AE•SER value for the cell sample PG comparison. (h) Determine each compared cell samples RCN value for the cell sample RNA put into the RT step of the assay, and determine the cell sample comparison RCNR value. (i) Determine the cell sample comparison cDNA related SCR value using the relationship (cDNA PG SCR)=(PG RCNR)×(PG AE•SER). (j) The PG mRNA ACE value for each cell sample can be determined from the relationship (PG mRNA ACE)=(measured PGo÷PG AE•SE)÷(PG RCN). Here, (measured PGo÷PG AE•SE)=(PG RN). Note that the above-described method for determining the assay cell sample SCR value for an SG primed RT-PCR assay, can also be used to determine the SCR value for oligo dT or random primed RT-PCR assays. Note also that the validity of the above-described method for SCR determination depends on the validity of the prior art belief and practice that in the same RT mix the AE•SE values for all particular gene and standard cDNAs is the same, or nearly the same. A recent report which uses a method related to the above-described method, suggests that said prior art belief and practice may not always be valid (111). Note further that the validity of the above-described SCR determination method requires knowing very accurate relative or absolute E values for the particular gene and/or standard.

In order to determine an RT-PCR measured particular gene RN or mRNA abundance value, which is biologically accurate, it is necessary to determine the number of particular gene ACEs which are present in the PCR amplification step. Prior art does not do this. In order to determine an RT-PCR measured particular gene comparison N-DGER value which is biologically accurate, it is necessary to determine the SCR value for the assay PCR amplification step. Prior art does not. In addition, prior art RT-PCR assay practice does not often determine the particular gene or standard AE•SE and AE•AE values for an assay. The prior art determinations of the assay AE•SE values for a particular gene or standard cDNA requires knowing precisely accurate amplification E values for the PG and S. When the E values are precisely accurate, small deviations from accuracy for an AE•AE value will cause only a small deviation from accuracy in the resulting AE•SE value. However, small deviations from accuracy for an E value can cause very large deviations from accuracy for the resulting AE•SE value. This can be illustrated by considering the effect of an E value, which has a measured value of 0.85±0.05 where one standard deviation is equal to 0.05, or about 6%. Here, when the PG AE•SE determination is done using a 30 cycle PCR assay for the measured 0.85 E value, the resulting PG AE•SE value can deviate from accuracy by as much as 2.3 fold too high, or 2.3 fold too low, depending on the actual PG E value for the assay. Thus, a 6% change in the E value can cause a 2.3 fold change in the measured PGo value and the measured PG AE•SE value. Note that prior art seldom determines the standard deviation value for a measured E value. When such measurement are reported, standard deviations of ±10-15% are common.

Interpretation of Measured Cell Sample SCR Values.

An essential aspect of determining the SCR value for a cell sample gene expression comparison is to obtain a quantitative measure of the number of each sample's cells or cell equivalents which are compared in the assay. As discussed earlier, an absolute or relative measure of cell number can be used for this purpose. Absolute determination of the number of cells in a cell sample is done by directly counting the number of cells in the cell sample. For a variety of reasons this can be very difficult or impossible to do, or impractical to do for multiple cell samples. A quantitative measure of the relative number of cells in compared cell sample can be done by quantitatively measuring a physical or chemical property or activity of the sample cells, which correlates accurately with cell number. Currently the best method for doing this is to measure the amount of cell DNA associated with a cell sample, and then determine the number of cells in the sample by dividing the measured amount of cell sample DNA by the known value for the amount of DNA per haploid or diploid cell for the cell type. Such values are known for many prokaryotic and eukaryotic cells. This DNA measurement method is the easiest and often essentially the only practical approach for obtaining a measure of the number of cells in a cell sample. Note that both the measured amount of DNA in the cell sample and the value for the amount of DNA per haploid or diploid cell, should represent the amount of DNA in intact cells. DNA per cell values derived from the amount of DNA isolated and purified from a known number of cells, can be known to be accurate only when the DNA isolation efficiencies are known and taken into consideration. Here, for convenience, the amount of DNA per cell will be determined in terms of the amount of DNA per haploid prokaryotic or eukaryotic cell. Note that in order to obtain an accurate SCR value based on the DNA per cell, the ploidy or average ploidy of the compared cells must be known.

While for a particular cell type the amount of DNA per haploid cell is the same for all haploid cells of that type, the amount of DNA per cell, or the ploidy of the cell can vary by as much as twofold, depending on the stage of the cell cycle the cell is in. Therefore, two different cells of the same type can vary in the amount of DNA per cell by about twofold. Mixtures of cells which are associated with different cell cycle stages generally have an average DNA content per cell of between 1-2 times the haploid content. Because the ploidy can vary with the cell cycle stage, and prior art values for the amount of DNA per cell almost always reflect the haploid or diploid DNA content per cell, the number of cells determined from the amount of DNA associated with a cell sample, can be overestimated by as much as twofold. When possible then, both the absolute and DNA based relative determination of the number of cells associated with a cell sample should be determined, and when the values differ, then the SCR value can be based on either the absolute value or the relative value. An SCR value determined from the absolute value, would result in the measurement of the quantitative gene expression activity per physical cell, or average physical cell, for a cell sample. An SCR value determined from the DNA based relative value, would result in the measurement of quantitative gene expression per haploid DNA complement, or average haploid DNA complement, for the same cell sample. Both of these measurements would be useful for understanding and interpreting gene expression mechanisms and dynamics.

Prior art does not determine the SCR value for cell sample comparisons. Due to technical and practical difficulties it is highly likely that many future SCR determinations will involve the DNA based relative method for determination of cell number. Here, such an SCR value is termed an R-SCR value, while the SCR based on the absolute cell number determinations is termed the A-SCR value. Particular gene N-DGER values obtained using R-SCR values can be directly compared to those obtained using A-SCR values, if the deviation from the haploid or diploid value which exists for the compared cells is known. If such deviation is not known, the compared values can differ by as much as twofold. The R-SCR value can be converted to an A-SCR value using the relationship (A-SCR)=(R-SCR)÷(the deviation of one cell sample from the haploid DNA content÷the deviation of the other compared cell sample from the haploid DNA content). This assumes the cell samples have the same haploid DNA content. Descriptions of cell sample gene expression comparison assay results should explicitly state whether the SCR value used is an A-SCR or R-SCR. Comparisons of particular gene expression results obtained from different assays should make explicit whether each compared result is associated with an A-SCR or R-SCR. Herein, unless otherwise noted SCR will refer to the A-SCR value.

Interpretation of Prior Art RT-PCR Measured Particular Gene RN, mRNA Abundance, and N-DGER Values.

Prior art oligo dT, random, and SG primed, RT-PCR gene expression comparison particular gene assay N-DGER values, are derived from a separately measured gene expression analysis particular gene RN value for each compared cell sample, or a separately measured particular gene mRNA abundance value for each compared cell sample. Prior art believes and practices that such prior art RT-PCR measured particular gene RN and mRNA abundance values are biologically correct within the assay measurement accuracy, and that particular gene N-DGER values derived from these particular gene mRNA transcript RN values and mRNA transcript abundance values, are biologically correct within the measurement accuracy of the assay. The vast majority of such prior art measured N-DGER values are derived from particular gene RN values, and relatively few are derived from mRNA abundance values. As discussed, most of these prior art RN and mRNA abundance values are highly likely to be biologically inaccurate. For such values, it cannot be assumed that because each compared particular gene RN value, or mRNA abundance value is biologically inaccurate, then the particular gene N-DGER value derived from them is also biologically inaccurate. In such a situation, the particular gene N-DGER value is very likely to be biologically erroneous, but absent further information, it cannot be known whether the particular gene N-DGER value is biologically accurate or not. As a result then, absent further information, which is not determined or known by the prior art, such a particular gene N-DGER value is uninterpretable with regard to biological accuracy. In a situation where the particular gene N-DGER value is derived from one biologically accurate, and one biologically inaccurate particular gene RN value or mRNA abundance value, the resulting N-DGER value is biologically inaccurate. Obviously, when the particular gene N-DGER value is determined from biologically accurate values for each compared particular gene RN or mRNA abundance, then the resulting particular gene N-DGER value is biologically accurate.

The discussion on the interpretation and validity of prior art RT-PCR measured particular gene RN values and mRNA abundance values concluded that almost all such prior art values are biologically erroneous. As discussed, this conclusion does not indicate that the particular gene N-DGER values derived from these erroneous and biologically incorrect mRNA transcript numbers, are also erroneous and biologically incorrect. The conclusion does indicate however, that such a prior art particular gene N-DGER value cannot be known to be erroneous and biologically incorrect or biologically accurate, absent further information not provided by the prior art. Such “further information” is discussed below in the context of the assay situation or situations required, in order that compared RN values or mRNA abundance values, which are biologically correct or incorrect, yield N-DGER values which are biologically correct.

In order to generate biologically accurate cell sample particular gene N-DGER values, compared prior art measured RN values must be associated with one of the following RT-PCR assay situations. For this discussion, an extent of quantitative deviation is always greater than one, and represents the multiplicative factor by which the measured value differs from the biologically accurate value. The qualitative extent refers to whether the measured value is greater than, or less than, the biological value. (i) Each compared RN value must be biologically accurate, and each compared cell sample intact cell RNA CE value must be the same. (ii) Each compared RN value must deviate from biological accuracy to the same quantitative and qualitative extent, and each compared cell sample intact cell RNA CE value must be the same. (iii) Each compared RN value differs in the extent of quantitative and/or qualitative deviation from biological accuracy, and the ratio of the compared cell sample intact cell RNA CE values compensates for the overall biological inaccuracy of the compared RN values, to generate an assay SCR value equal to one, and a biologically correct particular gene N-DGER value.

In order to generate biologically accurate cell sample comparison particular gene N-DGER values, compared prior art mRNA abundance values must be associated with one of the following assay situations. (a) Each compared mRNA abundance value must be biologically accurate. (b) Each compared mRNA abundance value must deviate to the same quantitative and qualitative extent from biological accuracy.

Absent further information it cannot be known whether any one prior art particular gene N-DGER value is associated with one of the assay situations which will produce a biologically correct N-DGER value or not. Such information includes, but is not limited to the following. The validity of each of the pertinent tacit assumptions. The compared cell sample's intact cell RNA CE values, and efficiencies of RNA isolation. The compared cell sample's particular gene and standard assay values for AE•SE and AE•AE. The compared cell samples SCR value associated with the assay PCR step. When such further information is absent, as it is for almost all, if not all prior art RT-PCR assays, the prior art RT-PCR measured particular N-DGER values are uninterpretable with regard to the biological accuracy of the quantitative value for gene expression differences and direction of changes in gene regulation.

Examples of Prior Art RT-PCR Assay Determination of Particular Gene mRNA RN Values, mRNA Abundance Values, and N-DGER Values.

In order to further illustrate the conclusions on the interpretation and validity of prior art RT-PCR assay measured particular gene mRNA RN values, mRNA abundance values, and N-DGER values, it will be useful to examine in some detail several examples of prior art RT-PCR assay results. Certain prior art reported RT-PCR assays use a known amount of cell sample T-RNA or mRNA which is claimed to represent a known number of sample cells, in the RT step of the assay. It is then claimed that biologically correct and interpretable particular gene RN values, and mRNA abundance values for the analyzed cell sample are obtained. Further, it is claimed that biologically accurate particular gene N-DGER values are obtained by comparing particular gene mRNA abundance values from different cell samples. Such prior art RT-PCR assays utilize either the earlier described Approach One or Approach Two.

One such prior art RT-PCR assay example (147) which utilizes Approach One is discussed below. For an approach one assay, known equal amounts of compared cell sample T-RNA or mRNA from cell samples which are claimed to have the same intact cell T-RNA or mRNA CE value, are used in the RT step of the gene expression comparison assay. This example first determines the measured RN values for a particular gene in different yeast cell samples. Each cell sample particular gene measured RN value is then converted to a particular gene mRNA abundance value by using one value for the amount of yeast T-RNA per yeast sample cell. The particular gene mRNA abundance values from different yeast cell samples, are then compared to determine the particular gene N-DGER value for the compared yeast cell samples. It is claimed that such a particular gene N-DGER value is biologically accurate to within ±1.2 fold, and that a particular gene RN value and mRNA abundance value for a cell sample, is biologically accurate to within a factor of two. No internal standard mRNA was used for this example, but an external standard was used to determine the measured RN and abundance values.

For this example, each individual gene expression analysis RT-PCR assay involved the following steps. (i) Isolate T-RNA from the yeast cell sample. (ii) Use 0.12 micrograms of

T-RNA from the yeast cell sample in the RT step of the assay. The example claims that for each yeast cell sample at a different growth stage, 0.12 micrograms of isolated yeast T-RNA represents 10⁵ yeast cells. In other words, the example believes and practices the first tacit assumption, i.e., yeast cells which are in different metabolic states contain the same amount of T-RNA per cell, 1.2 picograms per cell. It is known that differences in yeast cell growth rates can be associated with 4 to 6 fold differences in the amount of T-RNA per cell. The example did not experimentally determine the value for the amount of T-RNA per cell for each yeast cell sample, but referred to a 1991 literature reference (206) as the source of the value. This reference claims that haploid yeast cells contain 1.2 picograms of T-RNA per cell, but does not indicate the growth stage of the yeast cells measured or the method of measurement used. The example does not measure the intact cell T-RNA CE value for each analyzed cell sample, but tacitly assumes that different yeast cell samples have the same T-RNA per cell content value, and further assumes that the accurate value is 1.2 picograms per cell for yeast cells at all stages of growth. Absent knowledge concerning the intact cell T-RNA CE values for each sample it cannot be known whether the first tacit assumption is valid for the assay, or whether the actual T-RNA CE value equals 1.2 picograms per cell. (iii) The example then produced the SG primed cell sample particular gene cDNA prep. The example does not determine the particular gene cDNA AE•SE, or the AE•SE value for the external standard used, and does not determine the number of particular gene ACEs produced in the RT step and put into the assay PCR amplification step. In addition, the example claims that the assay measured particular gene RN values and mRNA abundance values are biologically correct within the measurement accuracy of the assay. In order for this to be true, the example must assume the external standard curve can be validly used for quantitation, and that the number of yeast cell sample particular gene cDNA ACEs which are produced in the RT step is equal to the number of cell sample RNA CEs present in the RT step. Neither the standard AE•SE or AE•AE values were determined for this assay, and it cannot be known whether the use of the standard is valid or not. However, it is highly likely that its use is not valid. Further, it is known that the prior art particular gene cDNA AE•SE values almost always equal significantly less than one, and generally equal 0.1 to 0.5, and therefore, it is known that the number of particular gene cDNA ACEs (the CCN), produced in the RT step is almost always very significantly less than the RCN value of 10⁵ cell sample RNA CEs present in the RT step. (iv) The entire synthesized particular gene cDNA prep is put into the PCR amplification step and amplified. The example does not know the number of cell sample particular gene ACEs, or CCN, present in the PCR step, and the particular gene CCN is almost always significantly less than the cell sample RCN value for the RT step. In other words, for the example the assay PGo value is almost always very significantly smaller than the assay cell sample particular gene RN value in the RT step. (v) From the results of the real time amplification assay the example determines the assay measured particular gene cDNA PGo value, which represents the number of particular gene cDNA AE molecules produced in the assay RT step, and then put into the PCR amplification step. The example believes that the assay measured PGo value is biologically correct and is equal to the particular gene RN value in the RT step of the assay. In order for this belief to be valid, the prior art must assume for both the particular gene and associated external standard RT-PCR assays, the validity of the version of tacit Assumption Three which is associated with this assay. The example uses an external mRNA standard curve to determine the quantitative particular gene RN value. As discussed earlier, this means that in order for tacit Assumption Three to be valid for this assay, the combination of four different assay values, the PG AE•SE and AE•AE values, and the S AE•SE and AE•AE values, each of which often differs significantly in value in an assay, must be associated with just the right assay values so that the value of the product of, (PG/S AE•SER)×(PG/S AE•AER), is equal to one. This is highly unlikely, and it is therefore highly unlikely that the third tacit assumption is valid for the example assays. As a result, it is highly unlikely that an example assay measured particular gene RN value is equal to the actual biologically accurate particular gene RN value associated with the 0.12 micrograms of yeast cell sample RNA which is present in the assay RT step. (vi) The example then uses the assay measured particular gene RN value to determine the assay measured particular gene mRNA abundance value, which is equal to, (particular gene RN value)÷(the particular gene RCN value for the RT step, which here is equal to 10⁵ RNA cell equivalents). The example believes and practices that the assay measured particular gene mRNA abundance values are biologically accurate. In order for this belief to be valid, the example must assume the validity of the first, second, and third tacit assumptions for the assay. The validity of the first and third tacit assumptions was discussed above. In order to determine a biologically accurate particular gene mRNA abundance value for a cell sample, a biologically accurate value for the intact cell T-RNA CE must be known in order to determine an accurate value for the number of sample cell T-RNA CEs, which are present in the RT step. Standard prior art practice for experimentally determining a cell sample T-RNA CE value is to isolate and quantitate the amount of T-RNA from a known number of cells, and then determine the amount of isolated T-RNA per cell. This value for the amount of isolated T-RNA per cell is then used to determine the number of T-RNA CEs which are present in a given amount of cell T-RNA. For this determination prior art does not determine or discuss the isolation efficiency of the T-RNA from the cell sample. It is well known that the T-RNA isolation efficiency is almost always equal to significantly less than one. Therefore, for almost all prior art assays, including the example, the second tacit assumption is invalid, and the prior art measured cell T-RNA or mRNA CE values are significantly underestimated. As a result, even if the first and third tacit assumptions were valid for the example, the assay measured particular gene mRNA abundance value has a high likelihood of deviating significantly from biological accuracy. (vii) To determine a particular gene comparison N-DGER value, the example compares the assay measured particular gene RN or mRNA abundance values for the compared cell samples. As discussed, it is highly likely that the example measured particular gene RN and mRNA abundance values are biologically inaccurate. However, it cannot be concluded that the particular gene N-DGER value derived from two biologically inaccurate particular gene RN or mRNA abundance values, is itself biologically inaccurate. Under certain circumstances, a biologically correct particular gene N-DGER value can be derived from biologically incorrect particular gene RN or mRNA abundance values, even when all three tacit assumptions are invalid for the assay. This can occur because the effect of the invalidity of one or two assumptions on the biological accuracy of an N-DGER value, can be cancelled or magnified by the effect of the invalidity of one or two of the other assumptions on the biological accuracy of the N-DGER. Such an event is possible, but not likely.

For this first prior art example, the effects of the invalidity of these tacit assumptions on the assay measured values for particular gene RN, mRNA abundance, and N-DGER values, are practically meaningful only if the effect of the invalidity causes one or more of these assay measured results to deviate significantly from biological accuracy. To be practically meaningful the magnitude of such invalidity effects should be equal to a significant fraction of, or greater than, the measurement accuracy of the example RT-PCR assay. The potential and probable magnitudes of such invalidity effects on the biological accuracy of example RT-PCR assay measured particular gene RN, mRNA abundance, and N-DGER values are discussed below. This first prior art example claims that the example RT-PCR assay is accurate for measuring particular gene mRNA transcript and mRNA abundance values to within ±2 fold, and the assay is accurate for measured particular gene N-DGER values to within ±1.2 fold. In this context, the potential and probable magnitudes of such tacit assumption invalidity effects on the biological accuracy of the example and other RT-PCR assay measured particular gene RN or mTN, mRNA abundance, and N-DGER values, are discussed below.

For this prior art example, absent information, which is not provided by the example, it cannot be known whether one or more of the three tacit assumptions is valid or not. As discussed however, it is highly likely that most, if not all, of the examples particular gene RN and mRNA abundance values, and particular gene comparison N-DGER values, are associated with two or more invalid tacit assumptions. Note that tacit assumption two is pertinent only to example assays which measure mRNA abundance values, while tacit Assumptions One and Three are pertinent to all example assays.

The example assumes that the T-RNA CE value is the same for yeast cell samples, which are associated with different growth rates, cell cycle stages, and metabolic states. In other words, the example assumes the validity of the first tacit assumption for the assays. The intact cell sample T-RNA CEs for the compared cell samples was not determined. However, it is known that the T-RNA CE value for rapidly growing yeast cells is 4-6 times that for slow growing yeast cells. For this example, it seems reasonable to estimate that a twofold or more difference in compared cell sample T-RNA CE values is not uncommon. The example assay measurement accuracy for particular gene comparison N-DGER values is claimed to be ±1.2 fold. For such an assay, a difference of 1.2 fold in the compared cell sample intact cell T-RNA CE values, can cause the assay measured particular gene N-DGER value to deviate from biological accuracy by 1.2 fold. Associating such a 1.2 fold deviation to the assay measurement accuracy deviation of ±1.2 fold will cause all the assay measured particular gene N-DGER values to either increase by 1.2 fold or decrease by 1.2 fold, since differences in the compared cell sample T-RNA CE values are associated with a global assay variable. This 1.2 fold change over the normal ±1.2 fold measurement accuracy, can readily cause a measured particular gene N-DGER value to falsely indicate that a significant difference in expression exists, when it does not. Alternatively, a different measured particular gene N-DGER value can be caused to falsely indicate that no significant expression difference exists, when one does exist. As discussed earlier, for a typical prior art cell sample gene expression analysis comparison, prior art indicates that the vast majority of assay measured particular gene N-DGER values are equal to one or nearly one. This occurs for all prior art prokaryotic or eukaryotic cell sample comparisons. For a typical mammalian cell sample comparison, prior art has indicated that roughly ten thousand different particular genes have assay measured N-DGER values of one or nearly one. For prior art prokaryotic cell sample comparisons this number is around 2 to 3 thousand. For such a population of particular gene assay measured N-DGER values, the ±1.2 fold increase or decrease associated with the invalidity of the first tacit assumption can affect the interpretation of a large number of particular gene N-DGER values in a typical assay.

This first prior art example assumes that the second tacit assumption is valid. It is known that the efficiency of RNA isolation from compared cell samples is almost always significantly less than one, and often ranges from roughly 0.3 to 0.7. It is also known that the RNA isolation efficiency is often significantly different for different compared cell samples. This example does not determine the cell sample RNA isolation efficiency. Such cell sample RNA efficiency values would cause the prior art measured cell sample T-RNA CE value to deviate from biological accuracy by about 1.5 to 3 fold. This underestimated T-RNA CE value will then cause the assay measured particular gene mRNA abundance values to be 1.5 to 3 fold lower than the biologically accurate value. For this example and for RT-PCR assays in general it seems reasonable to estimate that a 1.5 fold difference in RNA isolation efficiencies is common for compared cell sample RNA preps. Such a difference can cause a compared cell sample measured particular gene N-DGER value to deviate by 1.5 fold from biological accuracy. Tacit Assumption Two is associated with a global assay variable, and as such will affect all particular gene N-DGER values in the same way. The example assay measurement accuracy for particular gene N-DGER values is claimed to be within ±1.2 fold. As discussed just above, this 1.5 fold change over the normal ±1.2 fold measurement accuracy, can readily cause the interpretation of many assay measured particular gene N-DGER values to be different relative to an assay situation where the second tacit assumption related 1.2 fold factor is not associated with the assay.

This prior art example assumes that the RT-PCR version of the third tacit assumption is valid for the example assay. Each example assay determined particular gene RN or mRNA abundance value, is associated with four third tacit assumption related assay factors, the particular gene AE•SE and AE•AE assay values and the standard AE•SE and AE•AE assay values. Note that the particular gene and standard PCR amplification E values are associated with the AE•AE values. It is known that the particular gene and standard AE•SE and AE•AE values can be different for different particular genes and different standards, and can be different for the same particular gene or standard in different cell samples. An assay AE•SE value is almost always equal to significantly less than one, and is generally equal to 0.1 to 0.5. This example did not determine the Particular Gene (PG) and Standard (S) AE•SE assay values. However, since cell sample cDNA prep AE•SE values of 0.1 to 0.5 are usual, it is reasonable to estimate that a cell sample cDNA AE•SE value of 0.25 to 0.5 is very common for the cell sample cDNA preps analyzed in this example. Further, it seems reasonable to estimate that the PG AE•SE and S AE•SE assay values, and the compared cell sample cDNA AE•SE assay values, commonly differ by 1.5 fold or more. It is also known that the assay values for PG AE•AE and standard AE•AE often vary significantly, and that the PG AE•AE assay values for compared cell sample cDNA preps, and the S AE•AE assay values for compared cell sample cDNA preps also often vary significantly. Note that a 6% difference in the assay E values for compared particular gene cDNAs will cause a twofold or so difference in the compared particular gene AE•AE assay values. The assay E values are only rarely measured for the particular gene and standards associated with a prior art assay. The available information suggests that prior art determined E values range from roughly 0.7 to 0.9, and that it is not unusual for E value differences of ±10 percent to occur. A ten percent difference in E values between a standard and particular gene in an assay will cause about a 5 fold difference in the AE•AE assay values for the particular gene and standard. Such difference can cause a fivefold deviation from biological accuracy for a particular gene RN or mRNA abundance value. It seems reasonable to estimate that a twofold AE•AE difference, that is a six percent difference in E values, for compared particular gene cDNA preps, or for compared particular gene and standard cDNAs, or for compared standard cDNAs, is common for prior art RT-PCR assays. For this example's assay and other prior art RT-PCR assays which utilize a standard to measure a particular gene N-DGER value, the effect of these assay AE•SE and AE•AE differences is complex. Such assays are associated with eight different third assumption related assay factors. These are the PG and standard AE•SE and AE•AE assay values associated with one cell sample, and the PG and standard AE•SE and AE•AE assay values associated with another compared cell sample. As discussed earlier, the third tacit assumption is valid for the example assay only when each of the eight assay factors has just the right assay value so that the ratio of, (the PG AE•SER value×the PG AE•AER value)÷(the S AE•SER value×S AE•AER value), is equal to one. This is highly unlikely to occur for an RT-PCR assay of any kind, and it is highly likely that the third tacit assumption is invalid for virtually all such RT-PCR assays. The effect of the invalidity of the third tacit assumption on the biological accuracy of assay measured particular gene N-DGER values is also complex. Such an effect could be small or very large, depending on the assay values for the PG and S AE•SE and AE•AE for each compared cell sample, and their interactions. It seems reasonable to estimate that a deviation from biological accuracy of 2 fold or greater occurs often for these assays, and that deviations as large as 10 or more are not uncommon. Such deviations would clearly be practically meaningful for this examples assay measurement accuracy of ±1.2 fold. Note that differences in the compared cell sample cDNA AE•SE values are incorporated into the assay PCR amplification step SCR value, while differences in AE•AE values affect the quantitative values for the assay measured particular gene RN and mRNA abundance.

For those example assays which measure cell sample particular gene RN values, and then compare cell sample RN values to determine an assay measured particular gene N-DGER value, only tacit Assumptions One and Three are pertinent for the assay. It is highly likely that for many such example assays both tacit Assumptions One and Three are invalid. The overall effect of the invalidity of both these assumptions on the magnitude of the deviation of the assay measured N-DGER from biological accuracy, is equal to, (the magnitude of the effect of the invalidity of Assumption One on the biological accuracy)×(the magnitude of the effect of the invalidity of Assumption Three on the biological accuracy). If one invalid assumption causes the measured N-DGER to be underestimated 1.5 fold relative to the T-DGER value, and the other invalid assumption causes an overestimated by 2 fold measured N-DGER value, then the magnitude of the overall effect is equal to (0.67)×(2) or 1.33. However, if each invalid assumption affects the measured N-DGER value in the same way, that is both cause an overestimation or underestimation, then the overall effect is a threefold (1.5×2), deviation from biological accuracy. Here, the overall deviation from biological accuracy is either 1.33 fold or 3 fold. The magnitude of each of these deviations is practically meaningful for this examples N-DGER assay measurement accuracy of ±1.2 fold.

For those example assays which measure cell sample particular gene mRNA abundance values, and then compare different cell sample particular gene mRNA abundance values to determine an assay measured particular gene N-DGER value, all three tacit assumptions are pertinent. It is highly likely that for many such example assays, all three tacit assumptions are invalid. The overall effect of these invalidities on the magnitude of the deviation of the assay measured N-DGER from biological accuracy, is equal to (the magnitude of the effect of the invalidity of Assumption One on the biological accuracy)×(the magnitude of the effect of the invalidity of Assumption Two on the biological accuracy)×(the magnitude of the effect of the invalidity of Assumption Three on the biological accuracy). Here for the example discussed, the maximum overall effect on the magnitude of the deviation of the assay measured particular gene N-DGER value from biological accuracy is equal to (1.5×1.5×2) or 4.5 fold. The minimum overall effect is equal to (0.67×0.67×2) or about 1.1 fold.

This prior art example used a known amount of yeast cell sample isolated T-RNA which is claimed to represent a known number of yeast sample cells, in the RT step of the assay. The example then claims that the assay measured particular gene RN value accurately reflects the number of particular gene mRNA molecules present in the claimed known number of T-RNA CEs, which are present in the RT step. The example then claims that a biologically accurate particular gene mRNA abundance value can be determined using the claimed known number of yeast T-RNA CEs which is present in the RT step. The example then further claims that such particular gene mRNA abundance values determined for different yeast cell samples can be compared to produce biologically accurate particular gene N-DGER values. For this prior art literature RT-PCR approach one example (147), the following conclusions can be made (i) Two or more of the three tacit assumptions are invalid for most, if not all, of these example assays. (ii) The standard prior art method for determining the amount of RNA per cell almost always produces significantly underestimated values. (iii) The number of yeast sample cells represented by the 0.12 micrograms of cell sample T-RNA which is put into the RT step is not known, and for a cell sample comparison the number of each cell sample's T-RNA CEs which are put into the assay RT step is not known for these example assays. (iv) The number of cell sample T-RNA CEs put into the assay RT step is almost always significantly less than the number of cell sample particular gene cDNA ACEs which are produced in the RT step, and the number of such ACEs is not known for these example assays. (v) The number of cell sample particular gene ACEs put into the assay PCR amplification step is not known for these example assays. (vi) The number of cell sample particular gene cDNA ACEs put into the assay PCR amplification step, is virtually always significantly less than the example believes is present. (vii) The example method for determining assay measured particular gene mRNA abundance values is invalid. (viii) For a cell sample comparison the compared numbers of particular gene ACEs is unknown, and the assay SCR value associated with the assay PCR amplification step is not known. (ix) The example assay measured values for a particular gene RN, mRNA abundance, or N-DGER, are very unlikely to be biologically correct, and absent further information which is not provided by the example, it cannot be known whether such values are biologically correct or not.

Essentially the same conclusions made for the first art literature RT-PCR assay example, can be made for a second prior art example which also uses approach one (146). This second example involves the use of oligo dT primed cDNA in a competitive RT-PCR assay. The example determined a cell sample T-RNA CE value from the amount of cell sample T-RNA isolated from a known number of cells, and then used this CE value to determine assay measured particular gene mRNA abundance values and N-DGER values derived from them.

Similar conclusions to those made for the first and second prior art examples, can be made for a third prior art RT-PCR assay example which utilizes the earlier described second approach. For the second approach an unknown amount of isolated cell sample RNA from a known number of cells, is put into the assay RT step. This third example utilizes oligo dT primed cDNA in a competitive RT-PCR format (145).

Certain of these conclusions can also be made for prior art literature examples of other non-RT-PCR related, non-microarray gene expression analysis assays (22, 144), which claim to measure the particular gene mRNA abundance values for different cell samples, and particular gene N-DGER values for compared cell samples. Among other concerns, these examples assume that compared cell samples have the same RNA content per cell, and the cell sample RNA isolation efficiencies are not determined and taken into consideration.

Determination of the PAFR Value.

While the PA mRNA fraction of the total cell RNA can be determined, practical methods do not exist for determining the quantitative value for the fraction of the total RNA of a cell sample which comprises the total mRNA fraction. The total mRNA fraction consists of the entire population of PA mRNA and PA⁻ mRNA molecules in the total RNA. It is however, possible and practical to determine for a cell sample total mRNA, the quantitative amount of a particular gene mRNA transcript which is present in the total RNA sample, and the quantitative amount of a particular genes total mRNA transcripts which is in the form of isolatable PA mRNA. This fraction is termed the particular gene mRNA PA fraction, or PAF.

The quantitative PAF value for a particular gene mRNA transcript which is present in a cell sample T-RNA prep, can be measured by using a labeled polynucleotide molecule prep which is specific for and complementary to the particular gene mRNA of interest, to first determine the amount of the particular gene mRNA which is present in the cell sample total RNA by well known saturation hybridization methods (187, 204). Then, after isolating the PA mRNA fraction from the total cell sample RNA, the same labeled polynucleotide and saturation hybridization method can be used to determine the amount of the particular gene mRNA which is present in the isolated PA mRNA, and in the total RNA preparation which has had the PA mRNA fraction removed and which contains the PA⁻ fraction of the particular gene mRNA complement. From these measurements the PAF for the particular gene mRNA in the particular cell sample total RNA, can be determined. The PAF for the particular gene mRNA in the cell sample total RNA, is then equal to the ratio of (the amount of particular gene PA mRNA present in a given amount of the total cell sample RNA)÷(the total amount of the particular gene mRNA present in a given amount of the total cell sample, or the ratio of (the amount of particular gene PA mRNA present in a given amount of total cell RNA)÷(the sum of the amount of particular gene PA mRNA present in a given amount of the total cell sample RNA and the amount of particular gene PA⁻ mRNA present in the same amount of the total cell sample RNA). One of skill in the art will recognize that methods are available for determining multiple different PAF values for different gene mRNAs in the same cell sample total RNA. For a particular gene comparison the ratio of, (the particular gene PAF value for one cell sample)÷(the PAF value for the same particular gene in another compared cell sample), is termed the PAF ratio, or PAFR.

Determination of cDNA Synthesis Yield Fraction (YF), and cDNA Synthesis Efficiency (SE), for A Cell Sample cDNA Prep.

For gene expression analysis and gene expression comparison assays a cell sample cDNA prep is produced in the assay RT step from cell sample template T-RNA or mRNA. The ratio for an RT step of, (the amount of cell sample cDNA produced in the RT step)÷(the amount of cell sample template RNA used in the RT step), is termed the cell sample cDNA synthesis yield field fraction value, or cDNA YF value for the cell sample cDNA prep. Prior art cDNA YF values almost always equal significantly less than one, and the YF values for assay compared cell samples are often significantly different. Here, the ratio of, (the cDNA YF value for one cell sample)÷(the cDNA YF value for the other compared cell sample), is termed the CYF ratio, or CYFR.

A cell sample cDNA YF value can be determined using well-known standard methods for quantitating the amount of RNA or DNA in a sample. These include, but are not limited to colorimetric, absobance, fluorescent, radioactive, and hybridization methods (1, 7, 13, 14, 19, 22, 187, 204).

It is also useful to describe a cell sample cDNA prep synthesis efficiency or cDNA prep SE. Here, the cDNA SE value is equal to the ratio of, (the number of cell sample cDNA prep CEs produced in the RT step)÷(the number of cell sample template RNA CEs used in the RT step). Methods for measuring the number of RNA CEs and cDNA CEs were discussed earlier.

Note that for the cDNA of a particular gene mRNA which is present in a cell sample cDNA prep, an oligo dT or SG primed particular gene mRNA cDNA Synthesis Efficiency (SE) is equal to the ratio of (the number of particular gene cDNA molecules produced in the RT step)÷(the number of particular gene mRNA transcript template molecules present in the RT step).

Determination of the Nucleotide Length of the Analyzed and/or Compared RNA Transcript LPN Preps.

There are a variety of established methods for measuring the relative or absolute nucleotide lengths of RNA and DNA molecules (7, 8, 13, 18, 204, 207, 208). These methods include denaturing and non-denaturing, gel electrophoresis, capillary electrophoresis, sucrose gradients, various other chromatographic methods, and mass spectrometry. These methods can be used to obtain for a cell sample mRNA or equivalents population, the average nucleotide length, and the distribution of the nucleotide lengths in the RNA or DNA preparations. These methods can also be used to determine for a particular gene RNA or DNA which is present in a cell sample RNA or DNA preparation, the nucleotide length and the distribution of the nucleotide lengths of the particular genes RNA or DNA molecules which are present in the cell sample RNA or DNA preparation. For this latter application the cell sample RNA or DNA is usually fractionated on a denaturing gel first, and then the location of the particular gene RNA or DNA of interest in the gel is identified using a single labeled polynucleotide, which is specific and complementary to the particular gene RNA or DNA of interest. The inclusion of molecular weight markers in the gel facilitates the determination of the nucleotide length of the RNA or DNA of interest. The well-known northern and southern blot analysis methods can be used for this purpose. A similar method can be used to determine the average nucleotide length and nucleotide length distribution profile for undegraded or degraded PA containing mRNA molecule populations. For this application labeled poly (dT) or poly (dU) is hybridized to the mRNA either before or after size fractionation in the presence of molecular weight markers. Such methods have been described in the prior art (204).

The above-mentioned methods can be used to determine the nucleotide length and nucleotide length distribution profiles of, cell sample total RNA and mRNA present in the T-RNA as well as isolated mRNA, and DNA, RNA, and mRNA LPN molecules of all kinds.

Determination of Nucleotide Sequence and/or Nucleotide Composition for Particular Gene RNA Transcripts or Particular Gene RNA Transcript LPNs.

A variety of well-known methods exist for directly determining the nucleotide sequence and/or the nucleotide composition of RNA or DNA samples. Unfortunately, it is not practical to directly determine the nucleotide sequence and/or nucleotide composition of particular gene mRNA, cDNA, or cRNA, LPN molecules, which are present in a cell sample mRNA LPN preparation. However, under certain conditions it is possible to infer the nucleotide sequence and nucleotide composition of a particular gene mRNA LPN which is present in a cell sample mRNA LPN preparation. Such inference requires a priori knowledge of the nucleotide sequence of the particular gene of interest. Ideally, the entire nucleotide sequence of the gene should be known, but under certain circumstances, a partial nucleotide sequence will suffice.

The inference is straightforward in the case of cell sample mRNA LPN preparations which are produced using oligo dT priming. Since the oligo dT primer always initiates the cDNA synthesis at the 3′ end of the mRNA molecule, which contains a polyadenylate sequence, all resulting cDNAs will represent at least the 3′ end of the mRNA molecule. If the mRNA template is undegraded and no other factor causes the synthesis to stop, the cDNA molecule will represent the entire nucleotide sequence of the template mRNA. If the mRNA template molecule is degraded, and therefore shorter than the undegraded template mRNA, or if the LPN synthesis is truncated for some reason, the resulting LPN will be shorter than a full length mRNA, and will not represent the entire template mRNA nucleotide sequence. The incomplete LPN however, is known to represent only the 3′ end of the undegraded mRNA template, which starts at the termination of the polyadenylate sequence, and ends where the synthesis is terminated. Thus, for such an mRNA LPN molecule, if the template mRNA sequence is known, and the nucleotide length of the synthesized LPN molecule is known, then the nucleotide sequence of the LPN molecule can be determined. The nucleotide length distribution of the population of incomplete LPN molecules will provide a measure of the range of the particular mRNA template's nucleotide sequences, which are present in the cell sample LPN preparation. The nucleotide composition of the particular LPN can then be determined from its known nucleotide sequence. This general inference process applies to both oligo dT generated cDNA and any cRNA derived from the cDNA. Clearly when the oligo dT produced mRNA LPN molecules which are produced are equal in nucleotide length to the mRNA templates used to produce them, then the nucleotide sequence and composition of each particular gene mRNA LPN molecule which is present is known, if the particular gene mRNA sequence is known.

The inference process is also applicable when cell sample mRNA LPN preparations are produced using specially designed gene specific primers of known sequence. A specially designed gene specific primer molecule consists of a single primer of known nucleotide sequence, which is specific for a particular gene mRNA template molecule, and which is designed to initiate LPN synthesis at a known nucleotide distance from the 5′ end of the template mRNA molecule. The LPN synthesis mixture can contain one or many different special specific gene primer molecules, and each particular mRNA template molecule in the mixture is primed at only one site, and on each said particular mRNA template molecule the priming site is the same or nearly the same number of nucleotides away from the 5′ end of the mRNA template molecule. Thus, in the LPN synthesis mixture, each particular gene mRNA template molecule is primed by only one primer molecule. Further, for each particular mRNA molecule in the RNA prep the priming site is the same nucleotide length distance from the 5′ end of the mRNA template. Alternatively, for different particular mRNA molecules in the RNA prep, the priming site may be a different known nucleotide length distance from the 5′ end of the mRNA template. The TPN for each particular gene mRNA LPN is equal to one. Here, the gene specific primer will initiate LPN synthesis at a particular site on the template mRNA, and the LPN synthesis will proceed from there. The resulting LPN molecule can represent the entire 5′ end of the template mRNA molecule. Alternatively, the resulting LPN molecule may be truncated and represent the region of the template mRNA sequence between the specific priming site and the site of synthesis termination, which may be short of the 5′ end. Thus, the resulting LPN molecule represents the portion of the mRNA template molecule nucleotide sequence between the start and termination sites, and will have a nucleotide length and nucleotide sequence which is equal to the nucleotide length and nucleotide sequence of said template mRNA portion. For such an mRNA LPN molecule, if the template mRNA nucleotide sequence is known, and the nucleotide length of the synthesized mRNA LPN is known, then the nucleotide sequence and nucleotide composition of the LPN molecule can be determined.

Note that for both the oligo dT and specific gene priming methods, the TPN=1 for all particular gene mRNA LPNs present in a cell sample LPN prep. Because of this, for each particular gene mRNA LPN in a cell sample LPN prep the nucleotide length or average nucleotide length is equal to the TNC or average TNC for the particular gene LPN molecule population. Here, then the TNC equals the nucleotide length of the particular gene LPN.

The above-described inference methods require that the nucleotide lengths of particular gene mRNA LPNs which are present in a cell sample LPN prep be known, or approximately known. As discussed earlier, if a cell sample LPN prep consists of particular gene mRNA LPN molecules which are essentially full sized, relative to undegraded template mRNA, then the nucleotide length of each particular gene mRNA LPN can be known, and the nucleotide sequence and composition inferred. In reality, a cell sample mRNA LPN prep rarely, if ever, consists exclusively of undegraded full sized mRNA LPN molecules. For these cell sample LPNs the average mRNA LPN molecule nucleotide length and nucleotide length distribution, can readily be determined by well-known methods. However, it is difficult if not impossible to determine from these average nucleotide length results, the nucleotide length and nucleotide length distribution of a particular gene mRNA LPN molecule population in the cell sample LPN prep. As discussed earlier, the nucleotide length of a very small fraction of particular gene mRNA LPNs present in a cell sample LPN prep, can be determined experimentally by well-known methods. Unfortunately, for the vast majority of particular gene mRNA LPNs it is impractical to directly determine their LPN lengths. Consequently, there is no practical prior art method for directly determining the nucleotide length for the vast majority of particular gene LPNs which are present in a cell sample LPN prep.

An approach which will allow the determination of the average nucleotide length and the nucleotide length distribution of a particular gene LPN which is present in the cell sample LPN prep is discussed below. The general approach involves the following. (a) Producing a cell sample LPN prep under conditions where the nucleotide length and nucleotide length distribution of each particular gene mRNA LPN in the cell sample LPN prep is the same or nearly the same. (b) Experimentally determining the average nucleotide length and nucleotide length distribution of the cell sample prep LPN molecules, or producing the cell sample LPN under conditions where the nucleotide length and nucleotide length distribution can be reliably predicted. (c) Using the nucleotide length results to infer the nucleotide sequence of one or more particular gene mRNA LPNs which are present in the cell sample LPN prep. This approach requires the ability to produce a cell sample mRNA LPN prep which consists of different particular gene mRNA LPNs which have the same, or approximately the same, nucleotide length and nucleotide length distribution. This can be done by producing the cell sample LPN under synthesis conditions which result in the synthesis of all of the particular gene mRNA LPNs being terminated in a controlled manner, at approximately the same nucleotide lengths. Such controlled termination can be accomplished by incorporating into the synthesis mixture one or more synthesis termination compounds. As an example, it is well-known that different dideoxy nucleotide triphosphates and other compounds cause the premature termination of DNA synthesis (209). Such a chain termination compound or compounds can be incorporated into a cell sample LPN synthesis mixture at a proportion or concentration, which will cause the synthesis of the cell sample LPN molecules to prematurely terminate at a particular average nucleotide length. In such a situation, essentially all of the cell sample particular gene mRNA LPN molecules will have the same or approximately the same, nucleotide length and nucleotide length distribution. After synthesis, the average nucleotide length for each cell sample LPN prep can be determined by established methods. The average nucleotide length of each particular gene LPN molecule in a cell sample LPN prep is then the same or nearly the same as the average nucleotide length of the cell sample LPN prep molecules. When the nucleotide length of a particular gene LPN is known, its nucleotide sequence, TNC, and nucleotide composition can be determined by inference, if the particular gene mRNA nucleotide sequence is known.

Random primers can also be used to produce cell sample mRNA LPN preparations. Random primer mixtures consist of a mixture of many different short oligonucleotide primers, each of which is targeted for a different mRNA template sequence or site of initiation. Therefore, random priming usually results in producing at least two or more different cDNA molecules per individual mRNA template molecule, and each of the different cDNA molecules has a different nucleotide sequence. In other words, for a particular gene mRNA cDNA or LPN molecule population which is present in the cell sample mRNA produced cDNA or cRNA LPN population, the TPN is virtually always ≧2. In addition, the TPN value can be different for different mRNA templates. Generally, the greater the nucleotide length of the particular gene mRNA template molecule, the higher the TPN for that mRNA molecule. The TPN for random primer produced particular gene mRNA LPN molecule populations can vary widely. In a cell sample mRNA LPN preparation made by the random priming of undegraded cell sample mRNA, a 300 nucleotide long mRNA can have a TPN of 1-2, while a 6000 nucleotide long mRNA can have a TPN of 20 or more.

For a particular gene mRNA template present in a cell sample total RNA or total mRNA preparation, the total nucleotide complexity of a population of randomly primed cDNA molecules represents almost the entire nucleotide sequence length of the particular gene mRNA template molecules which are present in the cell sample RNA preparation. This occurs even when the particular gene mRNA which is present in the cell sample RNA prep is not intact, and is present as significantly smaller RNA molecules than the undegraded particular gene mRNA molecule. This can be illustrated with the following example. Consider a particular gene mRNA which has an undegraded nucleotide length of 2000 nucleotides. In one cell sample total RNA preparation the particular gene mRNA is undegraded, while in a second cell sample total RNA preparation, the entire particular gene mRNA is present, but in a degraded form, where the average length of the individual particular gene degraded mRNA molecules is about 500 nucleotides long. The randomly primed particular gene mRNA cDNA which is produced from both the undegraded and degraded cell sample total RNA preparations, will represent essentially the entire particular gene mRNA nucleotide sequence. Therefore, if the particular gene mRNA nucleotide sequence is known, then the nucleotide sequence and nucleotide composition of the particular gene mRNA cDNA which is present in the cell sample mRNA cDNA preparation is also known. Note that the extreme 3′ end of a particular gene mRNA molecule will be somewhat underrepresented in the random primer produced particular gene mRNA cDNA population. Generally then, when random priming is used to produce a cell sample total RNA cDNA preparation, the nucleotide sequence and nucleotide composition of each particular gene mRNA cDNA population present in the cell sample cDNA preparation can be known, if the particular gene mRNA nucleotide sequence is known. This will occur even for highly degraded total RNA if the proper random primer conditions are used, but will not occur for very highly degraded total RNA.

When cell sample LPN preps are produced by random priming of the total RNA, the TPN of each particular gene mRNA LPN is generally equal to two or greater, and the TNC of each particular gene mRNA LPN is essentially equal to the TNC of the particular gene's undegraded mRNA template. Here, if the nucleotide sequence of the undegraded particular gene mRNA is known, then the nucleotide sequence and nucleotide composition of the particular gene mRNA LPN is also known by inference. In this situation, the particular gene mRNA LPN nucleotide sequence can be inferred without determining the nucleotide length of the particular gene LPN.

Random priming is also widely used to produce cell sample mRNA cDNA preparations from cell sample total PA mRNA isolated from total RNA. Here, if the mRNA present in the total RNA is degraded, the isolated PA mRNA fraction will represent only the 3′ end of each particular gene mRNA molecule. In such a situation, random primed cDNA produced from such degraded cell sample PA mRNA will not represent essentially the entire nucleotide sequence of a particular gene mRNA, but will represent only the 3′ end portion of each mRNA which is present in the sample. For random primed LPN molecules, such 3′ end nucleotide length is almost always considerably shorter than that for the mRNA template molecule used to produce it. Because of this the total PA mRNA LPN prep measured average nucleotide length cannot be used to determine the nucleotide length of a particular gene LPN which is present in the total PA mRNA LPN prep. In addition, the nucleotide length of the particular gene 3′ end mRNA template which is present in the isolated mRNA prep must be known in order to know the nucleotide sequence and TNC of the particular gene's random primer produced LPN. The nucleotide length of the particular gene mRNA which is present in the isolated mRNA prep can be determined by using established gene expression analysis methods, such as northern blot analysis. As discussed earlier, only a limited number of particular gene mRNA nucleotide lengths can be determined in this way. When for a particular gene 3′ end mRNA fragment, the nucleotide length is known, the TNC of the particular gene's random primed LPN is known and the nucleotide sequence of the LPN can be known by inference, as discussed above. Note that for random primed cell sample mRNA LPN produced from isolated mRNA from degraded total RNA, the TNC of a particular gene mRNA LPN can only be obtained by knowing the nucleotide length in the isolated mRNA prep of the particular gene mRNA template. In this situation, there is no general method for obtaining such a mRNA nucleotide length for all particular gene mRNAs in the isolated mRNA prep.

Determination of the Total Nucleotide Complexity (TNC) for A Particular Gene RNA Transcript LPN.

For a particular gene mRNA LPN produced by oligo dT or gene specific priming, the TNC of the LPN is equal to the nucleotide length or average nucleotide length of the cDNA or cRNA LPN molecules. The previous section describes methods for determining the nucleotide length and nucleotide length distribution of particular gene LPNs.

For particular gene mRNA LPN molecules produced by random priming of isolated cell sample total RNA, the TNC is essentially equal to the TNC of the undegraded particular gene mRNA molecule. This was discussed in an earlier section.

For particular gene mRNA LPN molecules produced by random priming of cell sample PA mRNA isolated from total RNA, the TNC is essentially equal to the nucleotide length of the particular gene PA mRNA template which is present in the isolated mRNA prep. In this case the PA mRNA template present in the isolated mRNA prep may or may not be shorter in nucleotide length than an undegraded particular gene mRNA molecule.

Determination of the Total Polynucleotide Number (TPN) for the Analyzed or Compared Particular Gene RNA Transcript LPN.

The average TPN for any particular gene mRNA or cDNA or cRNA molecules present in a cell sample mRNA LPN preparation is equal to the ratio of (the TNC for the particular gene LPN molecules in the cell sample LPN preparation)÷(the average nucleotide length of the particular gene mRNA LPN molecules which are present in the cell sample LPN preparation).

As discussed earlier the TPN=1 for particular gene mRNA LPNs produced by oligo dT or gene specific priming, and the TPN is almost always equal to two or greater for particular gene mRNA LPNs produced by random priming.

Prior art microarray and non-microarray practice often produces cell sample mRNA LPN preparations and then treat the LPN preparations to significantly reduce the nucleotide length of the LPN cDNA or cRNA molecules. In such a situation, required measurements and determinations should be made before treating the cell sample mRNA LPN preparation.

Determination of the Total Signal Activity (TSA) for the Analyzed or Compared Cell Sample RNA Transcript LPN Prep.

The TSA is measured in terms of the quantity of label signal activity per microgram of LPN, when the label signal activity is measured under the gene expression analysis assay conditions. A variety of well-known methods are available for quantitatively determining the amount of RNA or DNA in a sample, such as an LPN preparation. These were discussed earlier.

The most common labels used for directly labeling LPN preparations are fluorescence and radioactivity. Well known methods are available for quantitating the fluorescent or radioactive label signal activity associated with either RNA or DNA which is in solution, or RNA or DNA which is immobilized on a surface, such as a microarray surface. Well known methods also exist for determining conversion factors which can be used to adjust a label signal activity value which was measured under non-assay signal detection conditions, to an accurate label signal activity value as measured under the assay signal detection conditions. For microarray and many non-microarray gene expression analysis methods, the assay label signal detection condition requires the detection and quantitation of the label signal activity from label molecules immobilized on the surface of a microarray device in a small spot. Such measurements have been made routinely in the prior art for label molecules associated with known amounts of immobilized RNA or DNA.

The most common label signal molecules which are associated with indirectly labeled LPN molecules are fluorescent and light scattering molecules such as macromolecule and nanoparticle label entities. Well-known methods are available for quantitating fluorescent or light scattering signal activity associated with known amounts of RNA or DNA immobilized on a surface. Once the assay TSA values for each compared LPN preparation is determined, the assay TSAR value for the comparison can be determined.

Determination of PSAR and LLSR Assay Values for Directly Labeled LPNs.

The vast majority of prior art microarray gene expression analysis assays involve the comparison of cell sample randomly labeled mRNA Type 1 LPN preparations. For such assays, the PSA and PSAR are UNFs which are associated with pertinent non-global assay variables. Therefore, for such assays the PSAR must be taken into consideration during the normalization process. In order to properly take the assay PSAR into account, it is necessary to know a measure of the quantitative value of the assay PSAR for each particular gene mRNA LPN comparison in an assay. For prior art comparisons of cell sample randomly labeled mRNA Type 1 LPN preparations, the PSAR is also pertinent, and PSAR values for particular gene mRNA LPN comparisons in the assay are not determined and taken into account during the normalization process.

The PSA is a measure of the quantitative label signal activity per microgram, or per nucleotide base, for a particular gene LPN molecule population in a cell sample LPN prep. The direct determination of the PSA value for a particular gene LPN which is present in a cell sample mRNA LPN prep, requires the determination of a quantitative measure of the amount of particular gene mRNA LPN present in a cell sample mRNA LPN prep, and the signal activity associated with the particular gene mRNA LPN. From these values, the PSA for the particular gene mRNA LPN can be determined. If this is done for compared cell sample mRNA randomly labeled Type 1 LPN preps, then the PSAR of the assay for the particular gene comparison can be obtained. At best the direct determination of the PSA value for each particular gene LPN which is present in a cell sample LPN prep, is not practical.

For the vast majority of particular gene mRNA randomly labeled Type 1 LPN comparisons, neither the PSA nor the PSAR can be determined by direct measurement. However, the PSAR values for particular gene LPN comparisons in an assay can be determined by inference, if certain conditions can be met. This is discussed below. For simplicity, the discussion is presented in terms of the incorporation into an LPN of a particular ribo- or deoxyribo-nucleotide base which is directly or indirectly associated with a particular label molecule. A particular unlabeled nucleotide base is here designated a base molecule or B molecule. A particular B nucleotide molecule which is associated with a particular label molecule is here designated as a BS molecule, while the same particular B nucleotide molecule which is associated with a different particular label molecule is designated a BT molecule.

It is useful to first discuss the Total Nucleotide Complexity or TNC, for a particular gene mRNA LPN molecule population. The Total Nucleotide Complexity (TNC) has been earlier defined for both oligo dT and specific gene primed particular gene LPNs, as well as random primed particular gene LPNs. Here, for a particular gene mRNA LPN comparison the ratio of (the TNC for the LPN in one cell sample's LPN prep)÷(the TNC for the LPN in the other compared cell sample's LPN prep), is termed the TNCR. Note that the TPN=1 for a particular gene mRNA LPN produced by oligo dT or specific gene priming, and consequently the TNC of the particular gene mRNA LPN is essentially equal to the nucleotide length of the particular gene LPN molecules. Therefore, the TNCR for such an LPN comparison is equal to the ratio of (the nucleotide length of one compared LPN molecule)÷(the nucleotide length of the other compared same particular gene LPN molecule). This ratio is herein termed the nucleotide length ratio or NLR for a particular gene LPN comparison. In contrast, for a random primed particular gene mRNA LPN, the TNC is not equal to the nucleotide length of the particular gene LPN molecule, but is equal to the TNC of the particular gene mRNA which is present in a cell sample's total RNA or isolated mRNA. For simplification, the term representative LPN molecule for a particular gene will be used to describe a particular gene mRNA LPN molecule population which has been produced by either oligo dT, specific gene, or random priming. The representative LPN molecule for a particular gene mRNA LPN is defined by the TNC of the particular gene mRNA LPN molecule population. For a particular gene LPN produced by oligo dT or specific gene (SG) priming, the representative LPN molecules nucleotide length or TNC, is essentially equal to the nucleotide length, or average nucleotide length, of the synthesized particular gene LPN molecules. For a particular gene LPN produced by random priming, the representative LPN molecule's TNC and equivalent nucleotide length is essentially equal to the TNC of the particular gene mRNA template it was produced from. Further, the cell sample mRNA template molecule(s) may be much shorter in nucleotide length than an undegraded particular gene mRNA molecule.

Herein, the total number of labeled or unlabeled BS or BTnucleotide molecules which are associated with a particular gene's representative LPN molecule is termed the B molecule number or BN, and for a particular gene LPN comparison, the ratio of (the particular gene BN value for one cell sample)÷(the particular gene BN value for a compared cell sample), is termed the BN ratio, or BNR. Herein, the number of labeled BS or BT molecules associated with a particular gene's representative LPN molecule is termed the B label molecule number, or BLN, and for a particular gene LPN comparison, the ratio of (the particular gene BLN value for one cell sample)÷(the particular gene BLN value for a compared cell sample), is termed the BLN ratio, or BLNR. Further, for a particular gene LPN the ratio of (the particular gene LPN BLN value)÷(the particular gene LPN BN value), is termed the B label density or BLD, and for a particular gene LPN comparison the ratio of (the particular gene BLD for one cell sample)÷(the particular gene BLD for the compared cell sample), is termed the BLD ratio, or BLDR.

In addition, for particular gene LPN comparisons which involve two different label types, when the label signal activities are measured under the assay signal generation and detection conditions, the ratio of (the signal activity of a known number of LPN molecules which contain a known number of BS molecules)÷(the signal activity from the same number of LPN molecules which contain the same known number of BT molecules), is termed the relative signal activity ratio or RSR. For cell sample LPN comparisons using only one label, the RSR is equal to one. The RSR may or may not equal one for an LPN comparison using two different particular labels, one for each different LPN sample. The RSR is obtained by comparing the signal activities of equal numbers of label molecules. It is also possible to define a reference signal ratio in terms of the ratio of signal activities from known, but not equal, numbers of label molecules. For all RSR measurements, it is necessary that label density effects be negligible or known. Well known methods are available for quantitating such label signal activity which is either in solution or on a surface (5, 7, 13, 30, 210, 211, 212).

Note that the above overall discussion applies directly to a wide variety of different direct and indirect labels and different natural and unnatural ribo-, deoxy-, and modified nucleotide bases of all kinds.

Inference Method One.

The PSAR values for particular gene mRNA randomly labeled Type 1 LPN comparisons can be determined by inference if the following conditions are met. (a) The nucleotide sequence of a particular gene mRNA is known. (b) Oligo dT or specific gene primers are used to produce the compared cell sample LPN preps. (c) Each cell sample LPN prep is labeled with the same label, or a different label. (d) For each particular gene mRNA LPN molecule population in the assay, the TPN of the synthesized LPN molecule population is equal to one. (e) The assay BLD values for particular gene LPN comparisons are known. (f) The nucleotide length and nucleotide length distribution is known for the compared particular gene LPNs. (g) From a, b, d, and f, the nucleotide sequence, nucleotide composition, BN, and TNC, of the particular gene mRNA LPN can be inferred. (h) From g the TNCR for the particular gene LPN comparison can be inferred. (i) From e and g, the BLNR for the particular gene mRNA LPN comparison can be inferred. (j) For two label particular gene mRNA LPN comparisons the RSR value for the assay is determined. (k) Label density effects should be negligible for each particular gene LPN comparison.

Conditions (a), (b), (c), (d), (e), (f), (j), (k), can be known by measurement and/or design. Conditions (g), (h), and (i) can be known by measurement and inference. Condition (a) can be known by measurement, and is known for many, if not most, mRNAs. Conditions (b), (c), and (d) can be known by design.

Condition (e) can be known by design and measurement. This can be done by knowing for each cell sample LPN synthesis mixture the ratio of (the concentration of the B labeled nucleotide precursor)÷(the total concentration of B unlabeled and labeled nucleotide precursor). This ratio is termed the Labeled Nucleotide Fraction or LNF, and the ratio of compared cell sample LNF values, is the LNFR. For a particular gene LPN comparison in a cell sample LPN prep comparison assay, the particular gene comparison BLDR value is equal to the assay LNFR value when the efficiencies of incorporation of the labeled and unlabeled B molecules into the compared LPN preps are the same. The use of this approach is straightforward with radioactive labels, since the incorporation efficiencies of the radioactive and non-radioactive precursor bases are the same. However, this is not the case for chemically modified precursor bases such as fluorescently labeled or indirectly labeled nucleotide precursors. As an example, Cy3 labeled nucleotide precursor has a different efficiency of incorporation into LPN than does the unlabeled nucleotide precursor. Further, the incorporation efficiency of the same nucleotide precursor labeled with Cy5 is different from both the Cy3 labeled same nucleotide precursor and the unlabeled nucleotide precursor. The incorporation efficiency for a labeled nucleotide precursor BS or BT molecule, is expressed in terms of the ratio of (the number of BS or BT labeled precursor molecules incorporated into the cell sample LPN prep÷the total number of the labeled and unlabeled B nucleotide precursor molecules incorporated into the LPN prep)÷(the LNF for the B nucleotide precursor in the labeling reaction mixture). This is termed the Labeled Nucleotide Precursor Incorporation Efficiency or Labeled Precursor Efficiency, or LPE, for a cell sample LPN synthesis reaction mixture. The ratio of the LPE values associated with compared cell sample LPN preps is termed the LPE ratio, or LPER. For those LPN labeling reactions where the LPE value is significantly less than one, it is necessary to determine the LPE value in order to determine the BLDR values for particular gene comparisons. Such LPE determinations are straightforward and can readily be done with prior art methods. Here then, when LPER≠1, for a particular gene comparison, (the BLD value)=(LNF)(LPE), and the (BLDR)=(LNFR)(LPER). Note that when each compared LPN is associated with a different label, then there are two BLD values, one for each label. Here, the BLD associated with labels S and T will be termed the BSLD and BTLD values respectively, and the ratio of compared BSLD and BTLD values is termed the BLDR.

In the event that it is necessary to determine labeling conditions which will provide known BSLD and/or BTLD and BLDR values for compared particular gene LPNs, exogenous standard (S) mRNA molecules of known nucleotide sequence nucleotide length, and nucleotide composition, can be used. One or more different S mRNA molecules which have different nucleotide lengths, nucleotide sequences, different base compositions, and known degrees of degradation, can be used in order to examine the effect of these factors on the particular S LPN BLD value under different LPN synthesis conditions. For such an analysis the nucleotide length, nucleotide sequence, nucleotide composition, and degree of mRNA degradation must be known for the S mRNA in order to infer the nucleotide sequence and nucleotide composition of the S mRNA produced LPN molecules as described earlier. In addition, the label signal activity associated with a known amount of each synthesized S LPN should be determined under two different conditions in order to determine if LD effects are associated with the LPN. Here, the quantitative measure of the label signal activity is determined for the synthesized LPN molecules. Then, a quantitative measure of the label signal activity is determined for the same sample of synthesized LPN molecules, after the LPN molecules have been converted to nucleotides, or very short oligonucleotides. Such a conversion will eliminate any signal activity quenching and or enhancement effects, which are associated with the synthesized LPN molecules. Prior art determination of ALD values for cell sample LPN preps does not include such a measurement. The label signal activity from the known amount of LPN can then be compared to a standard curve of signal activity versus quantity of unincorporated label, in order to determine the number of label molecules associated with a known amount of synthesized LPN. From this, and the representative LPN molecule nucleotide length or TNC, the number of label molecules per microgram of the S LPN ad per representative S LPN molecule can be determined. This value, coupled with the nucleotide sequence and nucleotide composition of the representative LPN molecule, can then be used to determine the LPE value, and to determine the LPE and BSLD and BTLD values for the particular test mRNA molecule for the LPN synthesis conditions used to produce the S LPN molecules. The LNF value is known by design for each different labeling condition. Here, significant LD effects associated with the synthesized LPN will result in a difference in the quantitative label signal activity measurement between the intact S LPN molecules, and hydrolysed “converted” LPN molecules. The magnitude of this difference is a measure of the magnitude of the LD effects, which are present. This same synthesized S LPN can be further characterized with regard to the LD effects related to hybridization kinetics, and the stability of the hybridized S LPN duplexes. Note that BSLD and/or BTLD, and BLDR values, do not reflect the hybridization kinetic or hybrid stability aspects of the LD effects. The above-described method of determining the BSLD and/or BTLD, and BLDR values, for compared S LPN preparations can be used to examine the effects of a variety of assay factors on BSLD and/or BTLD and BLDR values, for compared particular gene LPNs. Such assay factors include RNA purity, nucleotide length, nucleotide sequence, nucleotide composition, label versus unlabeled B nucleotide ratios, nucleotide sequence secondary structure, type of label, and others. Such analyzes can be used to design LPN synthesis conditions, which allow the accurate prediction of BSLD and/or BTLD, and BLDR values for particular gene mRNA LPNs, and cell sample LPN preps, of many, if not all kinds. Note that for determining the effect of RNA purity on the BSLD and/or BTLD, and BLDR values, an exogenous mRNA can be mixed with a cell sample total RNA or isolated mRNA prep, and then a specific gene primer for the exogenous mRNA can be used to produce only exogenous mRNA LPN molecules for analysis. A second approach for determining the BLD and BLDR values for different particular gene LPN comparisons in a cell sample LPN prep assay involves the use of the previously discussed Average Label Density (ALD) values for the compared LPN preps. The ALD is a measure of the average number of label molecules per base for an entire cell sample LPN prep. Prior art methods can be used to determine the ALD value for a cell sample LPN prep labeled with B labeled molecules. From the ALD value the total number of B label molecules associated with a cell sample LPN prep can be determined. If the nucleotide composition of the cell sample LPN prep is known, the BLD value for the LPN prep can be determined. Here then, the BLD for a cell sample LPN prep is equal to the ratio of (the cell sample LPN prep ALD value)÷(the fraction of the total number of nucleotides in the cell sample LPN prep consisting of the B nucleotide). Such a BLD represents the average BLD value for the cell sample LPN prep, and is here termed the A•BLD. For a cell sample LPN prep comparison the A•BLD ratio is termed the A•BLDR. Note that for a cell sample LPN prep comparison where it is known that the base compositions of each compared LPN prep are the same, the A•BLDR=ALDR. Most prior art gene expression comparison assays involve the comparison of cell samples of the same organism type, such as human, mouse, or a particular prokaryote or eukaryote. Prior art believes and practices, and the prior art assay results indicate, that for such cell sample comparisons a very large fraction of the particular gene mRNAs for each cell sample are also expressed in the compared cell sample, and also have about the same abundance level in each compared cell sample. In addition, for such cell sample mRNA comparisons, the average nucleotide lengths and nucleotide compositions of compared cell sample undegraded mRNA preps are the same or nearly the same. In addition, the nucleotide length distributions of the compared cell sample undegraded mRNA preps are the same or nearly the same. Because of the similar properties of the compared cell sample mRNAs, it is reasonable to believe that for a cell sample LPN comparison the nucleotide compositions of the compared LPN preps are the same or nearly the same, and that the A•BLDR=ALDR. For such a cell sample LPN comparison assay, when the nucleotide lengths, nucleotide sequences, and nucleotide base compositions are the same or nearly the same, the particular gene comparison BLDR value is equal to the A•BLDR value for the comparison. Condition (f) can be known by measurement as discussed earlier in the section on measurement of the nucleotide lengths of LPN molecules. Condition (f) can also be known by design and measurement using the controlled LPN termination method, and measurement of the nucleotide lengths of the resulting truncated LPN molecules, as was earlier discussed.

Condition (g) can be inferred using the information from a, b, d, and f. Because of condition b, condition d is met and the TPN=1 for each particular gene mRNA LPN in the cell sample LPN prep, and it is known that the LPN molecules comprising each particular gene mRNA LPN populations represent only the 3′ end of the mRNA template used to produce it. Condition f then indicates the nucleotide length and TNC of the particular gene mRNA LPN molecules which represent the 3′ end of the particular gene mRNA molecule. From the known nucleotide sequence of the particular gene mRNA molecule, and the nucleotide length of the same particular gene's LPN molecules, the sequence of the said mRNA representative LPN molecule can be determined. From the nucleotide sequence the nucleotide composition of the mRNA representative LPN molecule is known.

Condition (h) can be determined for a particular gene LPN comparison by utilizing the TNC value for each particular gene mRNA representative LPN molecule in order to determine the TNCR value for the particular gene LPN comparison, as discussed earlier.

Condition (i) can be determined from conditions (e) and (g). The BLNR value for a particular gene LPN comparison can be determined from the earlier discussed relationships between BLDR and, BNR, BLNR, LNFR, NLR, and LPER, ALDR, A•BLDR. Such relationships include, but are not limited to, the following: (BLNR)=(BLDR)(BNR); (BLNR)=(LNFR)(LPER)(BNR).

Condition (j) can be known by measurement as discussed earlier. Condition (k) can be known by design and measurement, as discussed earlier.

When the BLNR, TNCR, and RASR values are known for a particular gene LPN comparison in an assay, the particular gene PSAR can be determined from the relationship (BLNR/TNCR)×(RSR)=(PSAR). When a single label is used, then (PSAR)=(BLNR/TNCR), unless the RSR≠1 due to array differences.

Inference Method Two.

Many prior art microarray and non-microarray gene expression analyzes compare cell sample randomly labeled Type 1 LPN preps which are produced by the random priming of isolated cell sample total RNA. For such a cell sample total RNA LPN comparison, the PSAR value for a particular gene mRNA LPN comparison can be inferred when the following conditions are met. (i) The mRNA molecules in the total RNA are not highly degraded. (ii) The nucleotide sequence of a particular gene mRNA is known. (iii) Random priming is used to produce each compared cell sample LPN prep. (iv) Each cell sample LPN prep is labeled with the same label or a different label. (v) The assay BLDR value for particular gene LPN comparisons are known. (vi) The TNC is known for each compared particular gene representative LPN molecule, and therefore the TNCR for the particular gene LPN comparison is known. (vii) From ii and vi infer the nucleotide sequence and nucleotide composition for each compared particular gene LPN. (viii) From v and vii infer the BN for each compared particular gene representative LPN, and the BNR and the BLNR for the particular gene LPN comparison. For an SGDS particular gene comparison the BNR equals one. (ix) For two label comparison determine the RSR. (x) Label density effects are negligible for each particular gene LPN comparison.

Conditions (i), (ii), (iii), (iv), (v), (vi), (ix), (x) can be known by measurement and/or design. Conditions (vii) and (viii) can be known by inference. Condition (i) can be known by measurement. Condition (ii) can be known by measurement, and is known for many mRNAs. Conditions (iii) and (iv) can be known by design. Condition (v) can be known by design and measurement. Condition (v) was discussed earlier as condition (e) for Inference Method One. Condition (vi) is met by design. For all but very highly degraded cell sample total RNA preparations, the TNC and nucleotide sequence of a particular gene mRNA molecule population which is present in the isolated total RNA preparation, is essentially the same as the TNC and nucleotide sequence of the particular gene intact mRNA molecule. In this situation, a particular gene mRNA LPN molecule population produced by properly designed random priming will virtually always have a TNC which is essentially equal to the TNC of the same particular gene mRNA molecule population which is present in the degraded or undegraded isolated cell sample total RNA. The TNC values for each compared particular gene mRNA LPN can be used to determine the TNCR value for the particular gene comparison, as discussed earlier. For SGDS particular gene comparisons the TNCR value equals one. The vast majority of random primed particular gene LPN molecules are significantly shorter in nucleotide length than the mRNA template, degraded or undegraded, used to produce them. Thus, a particular gene mRNA template usually produces multiple different LPN molecules per template, and most mRNA templates are represented by more than one different LPN molecules, each of which contains a different or partially different nucleotide sequence. A consequence of random priming is that the extreme 3′ end of a mRNA molecule will be somewhat underrepresented in the resulting LPN molecule population, relative to other regions of the same mRNA molecule. As a result of the random priming process, condition (vi) can virtually always be met, providing that the cell sample total RNA used to produce the LPN, is not very highly degraded. One of skill in the art will recognize that doing the random priming at a proper ratio of primer to template is necessary to meet the above conditions.

Condition (vii) can be inferred from conditions (ii) and (vi). As discussed in (vi) the TNC of the particular gene mRNA representative LPN molecule is essentially equal to the TNC of the particular gene intact mRNA molecule. Therefore, the nucleotide sequence of the particular gene mRNA LPN represents essentially the entire particular gene mRNA nucleotide sequence, and the nucleotide sequence and nucleotide composition of the particular gene mRNA representative LPN molecule can be inferred from the known nucleotide sequence of the particular gene mRNA.

Condition (viii) can be inferred from conditions (v) and (vii). By determining the BN value for each compared particular gene LPN, and the BNR value for the comparison, and then using the BNR value to obtain the BLNR value for the particular gene LPN comparison. This process was discussed in detail in the section concerning Inference Method One.

Conditions (ix) and (x) can be known by measurement and design, as discussed earlier.

When the BLNR, TNCR, and RSR values are known for the particular random primed mRNA LPN comparison, the (PSAR)=(BLNR/TNCR)×(RSR). For a single label then, (PSAR)=(BLNR/TNCR).

Inference Method Three.

Many prior art microarray and non-microarray gene expression assays compare cell sample LPN preps produced by the random priming of cell sample isolated mRNA or PA mRNA. The PSAR values for the comparison of particular gene mRNA randomly labeled Type 1 LPNs which are present in cell sample LPN preps which have been produced by random priming, can be inferred when the following conditions are met. (a) The mRNA molecules present in the cell sample isolated mRNA prep are not highly degraded. (b) The nucleotide sequence of a particular gene mRNA is known. (c) The nucleotide length and TNC of the particular gene mRNA molecule population in the cell sample isolated mRNA prep is known. (d) Random priming is used to produce each compared cell sample LPN prep. (e) Each cell sample LPN prep is labeled with the same label or a different label. (f) The assay BLDR values for particular gene LPN comparisons are known. (g) The TNC is known for each compared particular gene representative LPN molecule, and therefore the TNCR for the particular gene comparison is known. (h) From (b) and (g) infer the nucleotide sequence and nucleotide composition for each compared particular gene mRNA representative LPN molecule. (i) From (f) and (h) infer the BN for each compared particular gene representative LPN, and the BNR and the BLNR for the particular gene LPN comparison. (j) For different labels determine the RSR. (k) Label density effects are negligible for each particular gene LPN comparison.

Conditions (a), (b), (d), (e), (f), (j), (k), can be known by measurement and/or design. Condition (c) can be known by measurement and inference. Conditions (g), (h), and (i) can be known by inference. Conditions (a) and (b) can be met by measurement as discussed earlier. Condition (c) can be met by measuring the nucleotide length of particular gene mRNA molecule populations, which are present in the isolated mRNA prep. This can be done directly for only a limited number of particular gene mRNAs which are present in an isolated mRNA prep. The nucleotide sequence for a particular gene mRNA molecule present in the isolated mRNA prep can be inferred from its nucleotide length and the fact that the particular gene mRNA molecule represents at a minimum, the 3′ end of the mRNA molecule. This has been discussed earlier. The TNC of the particular gene mRNA molecule can then be inferred from its nucleotide length or nucleotide sequence. Under certain conditions the nucleotide length and TNC of particular gene mRNA molecules present in an isolated mRNA prep can be inferred from the measurement of the average nucleotide length of the total population of mRNA molecules which comprises the isolated mRNA prep.

Conditions (d) and (e) are known by design. Condition (f) is known by measurement and design as discussed earlier. Condition (g) can be known by inference from conditions (b), (c), and (d), as has been discussed in the section on Inference Method Two. Condition (h) can be known by inference as discussed in the section on Inference Method Two. Condition (i) can be known by inference as discussed in the section on Inference Method Two. Condition (j) can be known by measurement, as discussed earlier. Condition (k) can be known by design.

When the BLNR, TNCR, and RSR values are known for a particular gene isolated mRNA random primed, randomly labeled, Type 1 LPN comparison, the assay PSAR=(BLNR/TNCR)×(RSR). When a single label is used then, (PSAR)=(BLNR/TNCR).

In the absence of label density effects the RSR is a global assay variable, and as such the RSR value is the same for all particular gene LPN comparisons which are associated with a cell sample LPN prep comparison, whether one or two labels is used. When one label is used, then the RSR value is always equal to one. When two labels are used then the RSR value is always equal to a definite value X, and X may not be equal to one.

In contrast, the particular gene mRNA LPN comparisons which are associated with a cell sample LPN comparison often do not have the same assay TNCR values or the same BLNR values. Thus, different particular gene LPN comparisons in the same cell sample LPN prep comparison can have different TNCR and/or BLNR values. Such differences can be caused by different factors. Differences in the nucleotide lengths of compared cell sample LPN preps can cause the TNCR and/or BLNR values for different particular gene comparisons in the same cell sample LPN prep comparison to differ significantly. As discussed earlier many microarray assay factors can cause a significant difference in nucleotide lengths to occur for the compared cell sample LPN preps, and such differences are not uncommon in prior art microarray practice. In addition, even in the absence of nucleotide length differences in a cell sample LPN prep comparison, other factors can cause the assay BLNR values to be different for different particular gene mRNA LPN comparisons in the assay. As an example, differences in the purity of the compared cell sample RNAs can cause this, as can differences in the incorporation properties of different labeled nucleotide precursor molecules during the LPN synthesis. Neither of these factors are rare in prior art microarray practice. These different factors can cause the assay TNCR and/or the assay BLNR to be different for different particular gene mRNA LPN comparisons in the same cell sample LPN prep comparison. This indicates that the TNCR and BLNR are associated with non-global assay variables.

Prior art believes and practices that differences in label signal activity for microarray compared particular gene LPNs, are associated with one or more global assay variables which are related to the labeling of the LPNs and/or the detection of the label signal activity in the assay. The prior art normalization process reflects this belief. For such prior art assays, the PSAR is not determined or considered for the normalization of assay results. The PSAR values of many prior art microarray assays can be known to be different for different particular gene LPN comparisons in the assay, and therefore it can be known that the PSAR is associated with one or more non-global assay variables. For many other prior art assays, it cannot be known whether the assay PSAR values are associated with a non-global assay variable or not. Consequently, it cannot be known whether one prior art particular gene comparison assay result needs to be normalized differently than another. When one or more non-global assay variables are associated with an assay's PSAR values, it is necessary to know the assay PSAR value for each particular gene LPN comparison in the assay, and then consider the assay PSAR value in the normalization process for the particular gene comparison result, in order to properly normalize for PSAR. As discussed earlier, such normalization can be difficult and complex, and at times impossible. This process can be greatly simplified for a particular microarray or non-microarray assay, when each particular gene assay PSAR value is associated only with global assay variables. As discussed (the assay PSAR)=(BLNR/TNCR)×(RSR). Here, the RSR is generally a global assay variable, while for prior art microarray practice, the BLNR and TNCR are often associated with one or more non-global assay variables. Both the BLNR and TNCR assay values can be influenced by differences in the nucleotide lengths or TNCs of the compared particular gene LPNs. While prior art does not take the nucleotide lengths and/or TNCs of the compared particular gene LPNs into consideration during the normalization process, prior art is aware that it is not unusual for such differences to occur. The BLNR assay value can also be influenced by differences in the efficiency of LPN labeling. The prior art normalization process rarely takes the efficiency of labeling into consideration and regards this factor as being a global assay variable. The prior art normalization process then, does not consider differences in the nucleotide length, the TNC, or the labeling efficiency, of the compared particular gene LPNs to be associated with non-global assay variables. The PSAR normalization process for particular gene LPN comparisons can be greatly improved over the prior art process by taking into consideration both the nucleotide length and/or TNC, and labeling efficiency factors, and also whether these factors are associated with global or non-global assay variables.

The PSAR normalization process for particular gene mRNA LPN comparison assay results can be greatly simplified by a combination of assay design and assay factor measurement. This can be done by designing the microarray or non-microarray assay so that both the TNCR and the BLNR for the assay act as if they were associated only with global assay variables. When this occurs the assay BLNR and TNCR values are the same or nearly the same for all particular gene comparisons in the assay. In this situation, since the RSR value is generally associated only with global assay variables, and since each particular gene comparison PSAR value=(BLNR×RSR)÷(TNC), the PSAR value for each particular gene comparison in the assay also acts as if it were associated only with global assay variables. Here, each particular gene comparison PSAR value in the assay will have the same PSAR value. The process of doing this varies somewhat for different assay formats. This is discussed below.

Many prior art microarray and non-microarray assays utilize oligo dT, or specific gene priming, to produce the compared cell sample LPN preps. Here, the TNCR value will be the same for all particular gene mRNA LPN comparisons in a cell sample LPN prep comparison, when the assay meets the following condition. For a compared cell sample LPN prep the nucleotide length and therefore the TNC, of each particular gene LPN molecule population is the same by design, and is known by measurement and/or design. The method of controlled termination of LPN molecules during synthesis, can be used to produce a cell sample LPN prep in which each particular gene LPN molecule has the same nucleotide length or average nucleotide length, and the same TNC. Nucleotide length measurement methods can be used to confirm this. In this situation, for a cell sample LPN prep comparison the TNC for each particular gene mRNA LPN comparison in the assay is the same, and is known. For this assay the TNCR value acts as a global assay variable, and the TNCR for each particular gene LPN comparison in the assay is equal to X, where X may or may not be equal to one.

The TNCR will also act as a global assay variable in a situation where oligo dT primer is used to produce for each compared cell sample only LPN molecules which have the same nucleotide length as the undegraded mRNA template it was produced from. Here, the TNCR=1 for each particular gene mRNA LPN comparison in the comparison assay. Specially designed specific gene primers targeted for the extreme 3′ end of each particular gene mRNA can also be used to produce essentially full sized mRNA LPN molecules for each particular gene mRNA.

For a situation where random primers are used to produce the compared cell sample LPN preps from total cell sample RNA, the TNCR=1 for each particular gene LPN comparison in the assay, and the TNCR acts as a global assay variable. Here it is desirable, but not necessary, that the nucleotide length of each particular gene mRNA LPN molecule population in a cell sample LPN prep be the same, or known.

The BLNR assay value can be influenced by differences in nucleotide length and/or TNC, and the efficiency of labeling of the compared LPN molecules. Thus, in order to ensure that the nucleotide length and/or TNC aspect of the BLNR acts as a global assay variable, the assay must be designed so that the TNCR also acts as a global assay variable, as described above. In order to ensure that the LPN labeling efficiency aspect of the BLNR acts as a global assay variable, it is necessary to design the assay so that the BSLD or BTLD value for each particular gene mRNA LPN which is present in a cell sample LPN prep is the same, and that the BLDR values for each particular gene LPN comparison in a cell sample LPN prep comparison are the same, and known. Methods of accomplishing this were discussed earlier. For an SGDS particular gene oligo dT primed LPN comparison for which the compared particular gene LPN nucleotide lengths are the same, the assay BLNR will act as a global assay variable.

A cell sample Type 2 LPN prep can be characterized by its LPN label molecule number or LLN. The LLN is the number of label molecules associated with each LPN molecule, short or long, which is present in the cell sample LPN preparation. The LLN is the same for each particular gene mRNA LPN molecule population in a cell sample Type 2 LPN prep, and is therefore a global assay variable. The ratio of the LLN values of compared cell sample Type 2 LPN preps, is the LLNR. The LLNR is also associated only with global assay variables. The LLN value for each cell sample LPN prep is readily known by assay design. A preferred design is to use oligo dT or SG primers for producing the cell sample LPN prep, which have the same number of label molecules attached to each primer molecule.

Because each LPN molecule in a Type 2 LPN prep is associated with the same number of label molecules, the label signal activity associated with each LPN molecule in the LPN prep is the same. Here, the LPN label signal activity associated with each LPN molecule in the LPN prep is termed the LLS. For a cell sample Type 2 LPN comparison, the ratio of (the LLS value for one cell sample)÷(the LLS value for the compared cell sample), is termed the LLSR. The LLS and LLSR are each associated with global assay variables. Therefore, for a cell sample Type 2 LPN prep comparison, the LLSR value is the same for all particular gene comparisons.

The determination of an LLS value for a cell sample type LPN prep requires determining the signal activity associated with a known number of the LPN prep's Type 2 LPN molecules. This can be done for Type 2 LPN molecules which are free in solution, or immobilized in a spot on an array surface with commonly employed prior art methods. An alternate approach is to produce an exogenous standard (S) DNA Type 2 LPN molecule which is labeled with the same type of label, and with the same LLN, as the cell sample LPN prep, and use this Type 2 S DNA LPN prep to determine the LLS value for the cell sample Type 2 LPN prep. This can be done for each compared cell sample Type 2 LPN prep. The measured LLS values for each compared cell sample Type 2 LPN preps can then be used to determine the cell sample Type 2 LPN prep comparison LLSR value. Determination of LLSR values will be discussed in more detail in a later section.

Compared cell sample Type 2 LPN preps can be designed so that the assay LLSR value can be ignored during the assay normalization process because the assay LLSR value is known to equal one or nearly one. This is done by using the same label for producing each compared type 2 LPN prep, and ensuring that the LLN value for each Type 2 LPN prep is the same. This can be readily done by using the same label associated primer oligo dT or SG primer molecules to produce each compared Type 2 LPN prep. For Type 2 LPN prep comparisons where each compared cell sample Type 2 LPN prep is labeled with a different label, and where the LLNR may or may not equal one, an assay LLSR value of one can be attained by adjusting the signal generation and detection conditions so that the LLS of each compared Type 2 LPN prep is the same.

The above discussions on PSAR and LLSR primarily emphasize SGDS particular gene mRNA transcript directly labeled LPN comparisons, and apply to either cDNA or cRNA LPN comparisons. Further, the discussions apply directly to SGDS, DGDS, and DGSS particular gene RNA of all kind cDNA or cRNA comparisons.

Determination of the ALD for A Cell Sample LPN Prep and the LD for A Particular Gene LPN.

For a cell sample LPN prep the average label density or ALD is measured in terms of the average number of label molecules per nucleotide base in a cell sample LPN prep. For a cell sample LPN prep comparison the ALD ratio or ALDR, is equal to the ratio of the ALD values of the compared cell sample LPN preps. Determining the ALD value for a cell sample LPN prep requires measuring the number of label molecules associated with a given mass of LPN and then converting the LPN mass to the number of LPN nucleotides. The prior art determines the number of label molecules which are associated with a known amount of LPN, by first determining the label signal activity which is associated with the LPN, and then converting the label signal activity to label molecules by using a standard curve which relates label signal activity to the number of label signal molecules (7, 13, 30, 158, 162). The label signal molecules used to establish the standard curve are generally individual label molecules which are not attached to an LPN molecule. Prior art often determines the ALD in this manner. This method of determining the ALD is valid only when LD effects related to label signal activity quenching or enhancement are not associated with the LPN prep being measured. If such effects are present in the LPN, the ALD value can be significantly under- or overestimated. The presence of such LD effects in the measured LPN sample can be eliminated by converting the LPN molecules to nucleotides or very short oligonucleotides before determining the label signal activity. Here, it is useful to measure the label signal activity of the LPN sample before and after converting the LPN molecules to nucleotides. A difference in the label signal activity values would signal the presence of LD effects in the LPN, and the magnitude of the difference in the label signal activities would provide a measure of the magnitude of the LD effects in the LPN. Such measurements can be determined by well established methods. It is not uncommon for prior art cell sample LPN prep ALD values to differ by three fold or more. Note that the assay ALD value represents the average number of label molecules per base for the entire population of LPN molecules which are present in a cell sample LPN prep.

The label density or LD value for a particular gene LPN which is present in a cell sample LPN prep, is measured in terms of the number or average number of label molecules per base present in the particular gene LPN molecule population. For a particular gene LPN comparison, the LD ratio or LDR, is equal to the ratio of the LD values of the compared particular gene LPNs. Note that the LD value for a particular gene LPN which is present in a cell sample LPN prep, may or may not equal the ALD value for the cell sample LPN prep. Determining the LD value for a particular gene mRNA LPN which is present in a cell sample mRNA LPN prep, requires the determination of a quantitative measure of the amount of particular gene mRNA LPN present in a cell sample LPN prep, and the number of labels associated with the particular gene LPN. From these values the LD for a particular gene LPN can be determined. While methods for measuring the number of labels per base are well known, the direct measurement of the number of labels per base for a particular gene LPN molecule population which is present in a cell sample LPN prep, is difficult at best. It is likely that such measurements can be done only for high abundance mRNA LPNs. The difficulties are similar to those discussed earlier for directly determining the PSA values for particular gene LPNs which are present in a cell sample LPN prep. For the vast majority of particular gene mRNA LPN comparisons in a cell sample LPN prep comparison, neither the LDs nor the LDRs can be determined by direct measurement.

As discussed earlier, when the assay LD effects are not negligible, the assay values for the non-global assay variable NFs, PSAR and/or PS-HKR and/or PSSR, can be influenced significantly. Therefore, accurate normalization of the assay RASR values for each of these NFs, requires the normalization of the assay result for the LD effect associated with each NF.

For the vast majority of particular gene mRNA comparisons in an assay, neither the LD nor the LDR assay values can be determined by direct measurement. However, the ALD value for the compared cell sample LPN preps can be determined directly for many microarray and non-microarray compared cell sample LPN preps. The ALD value is a rough measure of the average LD value for the particular gene LPNs which are present in the cell sample LPN prep. As such it can be used as a rough diagnostic for estimating whether the LD value is low enough so that the LD effects are negligible in the assay. If the LD effects are not negligible, then the ALD value can be used to estimate the magnitude of the LD effects on the relevant assay variables PSAR and/or PS-HKR and/or PSSR. Such use of the ALD value requires knowledge of the quantitative relationship between ALD and LD values in a cell sample LPN prep, and a quantitative relationship between the LD values and the LD effects on PSAR and/or PS-HKR and/or PSSR. Such relationships can be established by experimentation using established methodology. Similarly, the ALD values for a cell sample LPN comparison can be used as a rough diagnostic for estimating the presence of LD effects and the magnitude of the particular gene LPN comparison LDR's which are associated with the assay.

The LD and LDR assay values for particular gene LPN comparisons in an assay can be determined by an inference and measurement process which is similar to the earlier described inference and measurement process for determining the PSA and PSAR values for particular gene LPN comparisons in an assay. This earlier process for determining the PSA and PSAR values assumed that LD effects were negligible for the LPNs. This assumption is not necessary for the determination of the LD and LDR values by inference and measurement, since the BSLD and/or BTLD values, and the BLDR values, can be known by design, or can be measured in a way which is not influenced by the LD of the LPN. Such measurement method involves determining the number of label molecules associated with a known amount of LPN after converting the LPN molecules to nucleotides or oligonucleotides in such a way that the label is not damaged. This method was discussed in the earlier section on the determination of PSA and PSAR values. The determination of the LD and LDR values for a particular gene LPN comparison by inference and measurement is discussed below.

For a particular gene LPN, the LD is equal to the number of label molecules per base for the particular gene representative LPN molecule. As discussed earlier, the BLN for the same particular gene LPN is equal to the number of label molecules per particular gene representative LPN molecule. Further, the TNC of the same particular gene LPN is equal to the number of nucleotides present in the nucleotide sequence of the particular gene representative LPN molecule. Therefore, (LD)=(BLN/TNC), and for a particular gene LPN comparison the assay value for the LDR=(BLNR/TNCR). The previous section on the determination of PSA and PSAR assay values, describes the determination of the BSLD and/or BTLD, and BLDR values associated with the labeling method or methods used to produce the compared LPNs, as well as the determination of BLN and BLNR assay values, and the TNC and TNCR assay values, for particular gene LPN comparisons in a cell sample LPN comparison, by inference and measurement. The BSLD and/or BTLD, and the BLDR values were determined under conditions where it was known that LD effects were negligible, as were the other values. For a particular gene LPN comparison which is associated with negligible LD effects, this same inference and measurement process can be used to determine the LD and LDR values for a particular gene LPN comparison in a cell sample LPN prep comparison assay. Here, the BLN, BLNR, TNC, and TNCR values, obtained by inference and measurement for a particular gene LPN comparison, can be used to determine the LD and LDR assay values for the particular gene LPN comparison.

By ensuring that the BSLD and/or BTLD values, and the BLDR value associated with the LPN labeling method are measured under conditions where the LD effects are negligible, the said inference and measurement method can be used to determine the LD and LDR assay values for a particular gene LPN comparison in an assay, whether or not the particular gene LPN comparison is associated with negligible or significant LD effects. Such LD and LDR assay values can then be compared to experimentally established information regarding the relationship between the LD value and the LD effects, in order to determine whether the LD effect is negligible, and if not, to determine the quantitative magnitude of the LD effect on the relevant assay variables. Such experimental information does not presently exist but can be obtained using established experimental methods.

The LD and LDR assay values are associated with non-global assay variables, and therefore different particular gene LPN comparison in an assay can be associated with different LD and LDR assay values. Normalization of a particular gene LPN comparison assay result for assay situations where the LDR≠1, is done indirectly through the PSAR and/or PS-HKR and/or PSSR non-global assay variable NF values. The non-global nature of these assay variable NFs makes it difficult to directly measure their assay values for each particular gene LPN comparison in a cell sample LPN prep comparison. Inference methods can be utilized to determine the assay PSAR and PS-HKR values for particular gene LPN comparisons in an assay under certain assay design conditions. Determining the PSSR assay values for particular gene LPN comparisons in an assay is however, problematic.

The most effective way to minimize LD effects and to simplify the normalization process for the PSAR, PS-HKR, and PSSR assay variable NFs, is through the design of the compared cell sample LPN prep comparison. Such design includes the production of the compared cell sample LPN preps. Two general design approaches can be used. These are discussed below.

One approach involves producing compared cell sample LPN preps which have low ALD values, and therefore low particular gene LPN LD values. Preferably the ALD and LD values would be low enough so that no LD effects related to signal activity quenching or enhancement, LPN hybridization kinetics, or hybridized LPN stability, are associated with either compared cell sample LPN prep. Prior art information suggests that for radioactive labels this preferred requirement is met for even high specific activity radioactive LPNs. Limited prior art information suggest that for the commonly used fluorescent labels Cy3 and Cy5, the ideal requirement is largely met at ALD values of roughly one label molecule in roughly 80 bases, or less. Many prior art compared cell sample LPN preps have fluorescent Cy3 and Cy5 assay ALD values of from one label molecule in 10 bases to one molecule in about 50 bases. The main motive for comparing such high ALD cell sample LPNs is to increase the detection sensitivity of the assay. Put differently, the high ALD values are used in order to decrease the just detectable mRNA LPN abundance level which can be detected in the assay. As discussed earlier this is especially desirable, and needed, for the large number of mammalian particular gene comparisons which are associated with low abundance mRNAs. An alternate assay design approach addresses this issue. This is discussed below.

Another assay design approach involves comparing cell sample LPN preps for which certain of the LD effects are negligible, while one or more particular LD effects are significant, but known, for each particular gene LPN comparison in the assay. As an example, the LD values for the compared particular gene LPNs may be designed to be low enough so that the label signal quenching and hybridized LPN stability aspects of the LD effects are essentially negligible, while the aspect concerned with the slowing of the LPN hybridization kinetics is significant, but has the same quantitative effect on the hybridization kinetics of each compared particular gene LPN. It is also desirable to design this assay so that the PS-HKR=1, for each particular gene LPN comparison in the assay. This is possible for SGDS particular gene comparisons. For a different design the LD values for the compared particular gene LPNs may be higher, but low enough so that the hybridized LPN stability aspect of the LD effects is negligible, while the aspects concerned with the LPN hybridization kinetic slowing and label signal activity quenching are significant, but have the same quantitative effect on hybridization kinetics and label signal activity quenching of each compared particular gene LPN. Here, it is also desirable to design the assay so that the PL-HKR=1 and the PS-HKR=1 for each particular gene LPN comparison.

Another assay design approach involves the comparison of Type 2 LPNs. When end labeled Type 2 LPNs are compared all of the LD effects are minimized or eliminated by design, except for the quenching effect which may occur at high LLN values. However, because the LLN is a global assay variable, for all particular gene comparison RASR values in the assay, such quenching effects will be normalized for by the global assay LLSR value.

Other designs are also possible. In addition, the designs for different assay formats and labels can be different. Generally, the LD effects for radioactive labels are far less than for fluorescent labels.

Determination of Compared Particular Gene LPN Hybridization Kinetic Differences.

Established methods exist for determining the hybridization kinetics of particular gene LPN molecules with complementary nucleic acids which are in solution or immobilized on a surface (186, 187, 188, 204, 213, 214). Such methods can be used to detect and quantitate basic hybridization kinetic differences for compared particular gene LPN molecules, which differences are not dependent on LPN concentration. However, this can be done only if the concentration of each compared gene LPN in the hybridization solution is known. If the concentration of each compared gene LPN is known, it is not necessary to do the particular gene LPN comparison analysis, since the purpose of a particular gene comparison is to determine the absolute or relative concentrations of each compared particular gene LPN which is present in the LPN comparison. Since each compared LPN concentration is an unknown, it is not possible to directly determine any intrinsic hybridization kinetic differences which may exist for the compared LPNs. However, such differences in hybridization kinetics can be determined for a particular gene LPN comparison in a cell sample LPN comparison, by a process of inference and measurement. This process involves first, knowing by design and/or measurement the nucleotide length and TNC for each of the compared gene LPNs, and using such information to identify whether an LPN nucleotide length difference exists for the compared particular gene LPNs, and the magnitude of such a difference. The nucleotide length and the information for a particular gene LPN comparison can then be used to infer the nucleotide sequences and nucleotide compositions of the compared LPNs, and to identify whether a nucleotide composition difference exists for the compared LPNs, and the magnitude of any such difference. This same nucleotide length, TNC, nucleotide sequence, and nucleotide composition information can also be used to determine whether an LD difference exists for the compared particular gene LPNs, and the magnitude of such a difference. The earlier described design, measurement, and inference process which allow the determination for a particular gene LPN of the nucleotide length and/or TNC, nucleotide sequence, nucleotide composition, and LD, can be used in this inference and measurement process for the determination of the hybridization kinetic differences for compared particular gene LPNs.

The design, measurement, and inference process is used to identify differences in nucleotide length, nucleotide composition, nucleotide sequence, and LD, which exist for a particular gene LPN comparison in an assay. A difference in nucleotide length can affect the assay PL-HKR value for the particular gene LPN comparison. A difference in nucleotide composition can affect the assay PS-HKR value for the particular gene LPN comparison. A difference in nucleotide sequence can also affect the assay PS-HKR value for the particular gene LPN comparison, if the nucleotide sequence difference causes a difference in the compared LPN hybridization kinetics due to sequence dependent secondary structure differences. Further, a difference in LD affects the LDR, and indicates that there may be LD effect differences for the compared LPNs. Such differences indicate that significant differences in the assay hybridization kinetics of the compared particular gene LPNs may exist in the assay. If such differences do not exist or are minimal, then for the particular gene LPN comparison, a significant difference in the compared LPN hybridization kinetics which is related to the intrinsic characteristics of the compared particular gene LPNs does not occur.

In order to determine, whether a particular difference in nucleotide length, and nucleotide composition actually causes a difference in the compared LPN hybridization kinetics, and the magnitude of the difference caused, it is necessary to establish the quantitative relationship between different nucleotide lengths and/or nucleotide compositions for an LPN, and the relative extent of the hybridization kinetic inhibition which occurs for the LPN. This can be done by experimentation using well established methods. In order to determine whether a particular difference in the LD's of the compared particular gene LPNs actually causes a difference in the compared LPN hybridization kinetics, and to determine the magnitude of the difference, it is necessary to establish the quantitative relationship between different LD values and the extent of hybridization kinetic inhibition which occurs for the LPN. This can be done by experimentation using established methods. In order to determine whether a particular difference in the nucleotide sequences of compared particular gene LPNs is associated with a significant difference in nucleotide sequence dependent secondary structure which may be strong enough to cause hybridization kinetic inhibition, the nucleotide sequence of each compared LPN can be evaluated for the potential to form such secondary structure. This can be done using well established nucleic acid structure analysis methods, and structure analysis software programs. Such secondary structure predictions can be experimentally evaluated in order to establish a quantitative relationship between the predicted secondary structures, and their effect on the hybridization kinetics of the LPNs.

The relationship between the relative LPN hybridization kinetics and the nucleotide length is a general relationship, and can be applied to different particular gene LPN comparisons. Similarly, the relationship between the relative LPN hybridization kinetics and the nucleotide composition of the LPNs, should also be a generally applicable relationship. The relationship between the absolute and relative LPN hybridization kinetics and the assay LD value, is a more complex relationship, and may be different for different LPNs associated with the same label, or the same LPNs associated with different labels, and may also be different for different nucleotide lengths. The relationship between nucleotide sequence secondary structure and hybridization kinetic inhibition, is not yet established, but is likely to be influenced by nucleotide sequence, nucleotide composition, nucleotide length, and assay conditions in general.

For all of the assay factors, nucleotide length, TNC, nucleotide composition, nucleotide sequence, and LD, differences between compared particular gene LPNs can be controlled, minimized, or eliminated, by assay design. As discussed earlier it is possible to design the assay so that the nucleotide lengths, the nucleotide sequences, the nucleotide compositions, and the TNCs, are known to be the same or nearly the same, for essentially all SGDS particular gene LPN comparisons in the assay. For such an assay design there can be no significant differences in nucleotide length, TNC, nucleotide composition, nucleotide sequence, and nucleotide sequence related secondary structure, for the compared particular gene LPNs in the assay. For this assay the PL-HKR=1, and the PS-HKR=1. If for such an assay the LD for each compared particular LPN is designed to be low enough so that there are no LD effects, then the LD effects associated with hybridization kinetic inhibition, label signal activity quenching, and hybridized LPN stability, will be negligible and can be ignored. This assay design eliminates the potential effect of the above discussed said differences by eliminating the differences. As also discussed earlier, it is possible to design the assay so that certain differences are eliminated and other are known. A variety of different design possibilities are available which allow the differences to be controlled and/or minimized, and known. Such designs also apply to SGDS, DGDS, and DGSS particular gene RNA transcript of any kind LPN comparisons.

Determination of ECDP.

The characteristics of a particular gene CDP and ECDP were discussed earlier. Prior art utilizes oligonucleotide CDPs for oligonucleotide microarrays, and cDNA CDPs for cDNA microarray. The microarray CDP for a particular gene is determined by design and/or experimentation for the vast majority of microarray analyzed genes (7, 215). Prior art has developed extensive rules and processes for the design and selection of particular gene CDPs. As discussed earlier a particular gene ECDP is defined in an assay by both the particular gene CDP and the particular gene mRNA LPN characteristics.

A particular gene CDP and ECDP is often designed to represent the 3′ end portion of the mRNA molecule. Such ECDPs are suitable for oligo dT primer produced LPNs, or LPNs which have been produced using a specific gene primer targeted for the 3′ end portion of the mRNA. Such 3′ end targeted ECDPs are less suitable for random primer produced LPNs. Because of the greater nucleotide length of cDNA ECDPs, cDNA microarrays are more suitable than oligonucleotide microarrays for detecting random primed LPNs. For maximum microarray assay detection sensitivity with random primed LPNs, the nucleotide length of the ECDPs should be as close as possible to the TNC of the particular gene mRNA LPN which it is detecting.

Determination of MLD and MLDR.

In order to determine the assay MLD value for a particular gene LPN comparison, the assay values for the following assay factors must be known or determined by measurement or inference. One factor is the nucleotide length or average nucleotide length of the particular gene LPN in the assay. Earlier sections describe the determination of such a nucleotide length by measurement, or by design, measurement, and inference. The second factor is the TNC of the particular gene LPN in the assay. Earlier sections describe the determination of such a TNC by measurement, or by design, measurement, and inference. The third factor is the ECDP for the particular gene LPN of interest. An earlier section describes the determination of the ECDP by a design process.

MLDR effects on particular gene LPN comparison assay results can be controlled, minimized, and/or eliminated by assay design. As discussed earlier there is no MLDR effect when a particular gene LPN comparison uses Type 2 LPNs. MLDR effects can also be eliminated by designing the assay so that each SGDS compared particular gene LPN has the same nucleotide length and nucleotide sequence and TPN value. Such a design was discussed earlier. Under these conditions the assay MLDR=1 for a particular gene LPN comparison. Determination of MLD and MLDR was extensively discussed earlier.

Determination of LLNR.

The LLN value for each compared cell sample Type 2 LPN prep is readily known from the number or average number of label molecules which are associated with the primer type used to produce each LPN prep. Thus, the assay LLN for each compared LPN prep is known by assay design. The assay LLNR is then determined from the LLN values for each compared cell sample LPN prep. The LLN is measured in terms of the number of labeled molecules associated with each LPN molecule.

LLSR Determination and Normalization for Direct Label Type 2 LPN Comparisons.

A cell sample direct labeled Type 2 LPN prep is composed of a population of LPN molecules each of which is associated with the same number of label molecules. Further, the TPN=1 for each particular gene LPN molecule population in the cell sample prep. For such a Type 2 direct label LPN prep, each spot CDP molecule can hybridize to only one LPN molecule, and each hybridization immobilized LPN molecule on each array spot is associated with the same number of label molecules.

The LLS value for a particular gene hybridization immobilized Type 2 direct labeled LPN molecule, is equal to the assay measured signal activity associated with the LPN molecule. Here all different particular gene spot immobilized LPN molecules from one cell sample are associated with the same number of label molecules. When only one label is used for a cell sample Type 2 LPN prep comparison and the LLN for each compared LPN prep is the same, then after the assay hybridization and post-hybridization wash step the number of label molecules associated with each hybridization immobilized LPN molecule on each compared microarray is the same. The signal activity associated with each spot on one array, and on the compared array, is measured under identical conditions. Here, it is reasonable to believe that for all spots on one array, the immobilized Type 2 LPN molecules will have the same LLS value. However, for fluorescent labeled LPN preps it cannot be assumed that the LLS value is the same for the LPN molecules on compared arrays since each cell sample Type 2 direct label LPN prep is labeled and processed separately, and it is known that changes in signal activity of fluorescent label can occur during the process. Because of this it cannot be assumed that the cell sample fluorescent LPN comparison LLSR=1, even when the LLNR=1, and the same label is used for each cell sample LPN. However, for the comparison of radioactive cell sample Type 2 LPN preps which each have the same LLN value and are labeled with the same radioactive label, the LLSR for the cell sample comparison assay is equal to one. When the same radioactive label is used for each compared cell sample Type 2 LPN prep, and the LLNR=Z, where Z≠1, then the cell sample comparison LLSR=LLNR=Z.

For the comparison of cell sample Type 2 fluorescent LPN's the LLSR can be obtained by comparing the signal activity associated with equal numbers of fluorescent LPN molecules. When equal numbers of each compared cell sample's fluorescent Type 2 LPN molecules are compared, (the LLSR)=(the total signal activity associated with one cell sample LPN)÷(the total signal activity associated with the compared cell sample LPN). For this method, the nucleotide lengths or average nucleotide lengths of the compared fluorescent Type 2 LPN preps must be known by either measurement or design. Such determinations were discussed earlier. This method can be used to obtain the assay LLSR value for cell sample Type 2 LPN comparisons which use only one label, or those which use two different labels.

LLSR Determination and Normalization for Indirectly Labeled Type 2 L-LPN Comparisons.

For simplicity an indirectly labeled LPN molecule is termed a ligand LPN or L-LPN. A cell sample indirectly labeled Type 2 L-LPN prep is composed of a population of L-LPN molecules, each of which is associated with the same number of ligand molecules, and each such immobilized L-LPN molecule can usually bind only one SGC molecule. Preferably the ligand molecules are attached to one end of the L-LPN molecule. Further, for each particular gene L-LPN molecule population in the cell sample L-LPN prep, the TPN=1. For such a Type 2 L-LPN prep, each spot CDP molecule can hybridization immobilize only one L-LPN molecule, and each hybridization immobilized L-LPN molecule on each array spot is associated with the same number of ligand molecules.

The LLS value for a particular gene hybridization immobilized Type 2 L-LPN molecule, is equal to a quantitative measure of the signal activity associated with each L-LPN immobilized SGC molecule. Here, all different particular gene spot immobilized L-LPN molecules are associated with the same number of ligand molecules, and the ligand molecules are generally all located at one end of the immobilized L-LPN molecule. When only one ligand type is used for a cell sample Type 2 L-LPN prep comparison, and the number of ligands per L-LPN molecule is the same for each compared L-LPN prep, then after the assay hybridization step and post-hybridization wash step, the number of ligands associated with each hybridization immobilized L-LPN molecule is the same for each spot immobilized L-LPN molecule on each compared array. Then, when the SGC molecular dimensions are sufficiently large, after the staining step only one SGC molecule will be associated with each spot immobilized L-LPN molecule on either compared array. Further, all of the spot immobilized SGC molecules on either compared array are essentially identical to one another. The signal activity associated with each spot on each compared array is measured under identical signal generation and detection conditions. Since the SGC molecules associated with each spot and each spot immobilized L-LPN molecule on either compared array are identical to one another, it is reasonable to believe that the signal activity associated with each immobilized SGC molecule on either compared array is the same or nearly the same. In other words, the assay LSS value is the same for all spot immobilized L-LPN molecules on either compared array, and the LLSR=1 for all particular gene L-LPN comparisons. When the LLSR=1 for a particular gene Type 2 L-LPN comparison, the LLSR can be ignored for normalization. Here the assay LLS value should be associated only with global assay variables, and it is reasonable to believe that the LLS values associated with compared arrays are the same. For a carefully done array comparison the SGC molecules and staining conditions and procedures are identical, and no known assay variables should be associated with these assay factors. For such an array comparison the arrays themselves, while known to be very similar, are not identical. Individual arrays are known to be associated with surface microheterogeneity, and such microheterogeneity also occurs between arrays. At present it is not believed that such surface microheterogeneity can significantly affect the LLS values for L-LPN immobilized SGC molecules. While this belief appears quite reasonable, there is little hard data to support it. Further, for comparisons of what are considered to be essentially identical cell sample Type 1 cRNAs, three fold or greater differences in total array intensities of compared Affymetrix chip signal intensities are not uncommon (178). Such total signal differences may indicate the existence of an array surface effect on the signal generating efficiency of immobilized SGCs.

It is possible to determine whether the LLS value varies significantly between different spots on the same array, or different arrays. This can be done as follows. (i) Produce a preparation of exogenous standard (S) Type 2 L-LPN molecules which are identical or nearly identical in nucleotide length, nucleotide sequence, and number and position of ligand molecules per L-LPN molecule. (ii) To each cell sample Type 2 L-LPN prep, just before hybridization add equal mole amounts of the S L-LPN. The amount of S L-LPN added should ensure a strong spot signal which is well above background and well below saturation. The compared hybridization solutions should be identical and the amount of L-LPN added should be enough to give a strong, but not saturating spot signal. (iii) Each hybridization solution is incubated with an array which contains identical replicate S CDP spots specific for the S L-LPN. Preferably such replicate spots should be made in a way so that the print tip and print plate CNF values are equal to one, or are not pertinent for the assay. Such S replicate spots should be located on each compared array in multiple locations, and in the proper number, in order to obtain statistically significant sampling of each arrays surface microheterogeneity. (iv) The compared arrays are incubated under identical conditions and temperature for the same time. (v) Post-hybridization washing and processing is identical for each compared array. At this point, the average number of hybridization immobilized S L-LPN molecules per S spot should be the same or nearly the same, for each compared array. Further, since each immobilized L-LPN molecule is associated with the same number of ligand molecules, the average number of S L-LPN associated ligand molecules per S spot is the same, or nearly the same, for each compared array. (vi) Each compared array is then incubated with identical aliquots of the same SGC containing stain stock solution, under identical staining conditions, at the same temperature for the same time period. The stain period is long enough to ensure maximal SGC binding to immobilized ligand. The SBNR should equal one for these staining conditions. (vii) The post-stain wash and processing is identical for each compared array. At this point the average number of S L-LPN immobilized SGC molecules per S spot should be the same or nearly the same, for each of the compared arrays. In addition all immobilized SGC molecules are associated with an S Type 2 L-LPN molecule which is identical in nucleotide length, nucleotide sequence, ligand number and position, and number of SGC molecules associated with it. (viii) The same signal generation and detection conditions are used to measure the signal activity associated with each S spot on each array. The TSS and RAS associated with each S replicate spot is determined. (ix) For each compared array the average RAS value per S spot is determined. (x) Significant differences in the measured RAS values for different replicate S spots within an array indicate the presence of within array spatial surface difference effects on the assay results. For such effects it is generally assumed that the measured RAS values for particular gene spots adjacent to an S spot, are affected to the same extent as the S spot RAS value. Therefore, the adjacent S spot RAS value can be used to normalize the adjacent particular gene RAS values for the spatial surface effects. This can be done by designating one particular replicate S spot RAS value on the array as the reference S spot, and using that S spot RAS value to normalize all other replicate S spot and particular gene comparison spot RAS values for the array spatial surface effects. Absent such effects, the ratio of (the RAS value of any S spot)÷(the RAS value of the reference spot, here termed S spot R), should equal one. As discussed, this ratio is termed the SRR. Using the S spot R, an SRR value is determined for each different replicate S spot on the array. The SRR associated with a particular S spot on the array can be used to normalize particular gene spot RAS values adjacent to it for the spatial surface effects. This is done using the relationship, (the SRR normalized particular gene RASR value)=(the assay measured particular gene RASR value)÷(the SRR value associated with the particular gene spot). (xi) The assay LLSR value is equal to (the average replicate S spot RAS value for one array)÷(the average replicate S spot RAS value for the compared array). This assumes that the overall spatial surface differences on each array are on average, the same or nearly the same. (xii) Normalize each particular gene comparison RASR value on the array for its adjacent SRR value. (xiii) Each SRR normalized particular gene RASR value is then normalized for the assay LLSR value. This is done using the relationship (LLSR and SRR normalized particular gene RASR value)=(SRR normalized RASR value)÷(LLSR).

Under the specified conditions for the above approach for determining the LLSR assay value, the SBNR=1 for each replicate S spot comparison and particular gene comparison, and can be ignored for normalization.

One of skill in the art will recognize that this described method is but one of multiple methods which can be used to determine the assay LLSR value.

SBNR Determination and Normalization.

The UNF SBNR is pertinent to Type 1 and Type 2 indirect labeled L-LPN assays. The SBN reflects the number of SGC molecules which can stably bind to an immobilized L-LPN molecule. For a particular gene immobilized type 1 L-LPN molecule the SBN is measured in terms of the SGC signal activity per L-LPN nucleotide which is associated with the SGC molecules bound to an L-LPN molecule. As discussed, a measure of the compared particular gene L-LPN SBN values is the ratio of, (the signal activity per nucleotide for one L-LPN)÷(the signal activity per nucleotide for the other compared L-LPN). This ratio is termed the SBNR. In order to directly determine the SBN value for a spot immobilized L-LPN it is necessary to know the following. (i) The nucleotide length or average nucleotide length of the spot immobilized L-LPN molecules. (ii) The signal activity of the SGC molecules associated with the spot immobilized L-LPN molecules. (iii) A measure of the number of immobilized L-LPN molecules in the spot. Here, (i) and (ii) can be experimentally determined, but the direct determination of (iii) is not practical. Thus, the direct determination of the SBN for a spot immobilized particular gene L-LPN molecule is not practical. However, it is possible and practical to determine the average nucleotide lengths and ligand densities for compared particular gene L-LPNs, and the signal activity of the SGC molecules associated with spot immobilized particular gene L-LPN molecules. This information can be used to infer the particular gene relative assay SBN values and the SBNR values. Such an SBNR determination requires the development of standard curves relating the relative signal activities associated with immobilized L-LPN molecules of known nucleotide lengths and constant known ligand density, and of varying known ligand densities and constant nucleotide length. A different set of standard curves will be required for each different SGC used, and possibly for each different staining method. For a particular combination of SGC types, ligand density or densities, L-LPN nucleotide lengths, and assay method, an SBNR value can be obtained from the standard curves. For a cell sample L-LPN comparison, a particular gene L-LPN comparison which uses the same assay method and SGC type(s) as the standard curve, and compares the same nucleotide lengths and ligand densities as the standard curve, the SBNR value will be essentially the same as the standard curve value. For an assay which compares cell sample L-LPN molecules which have the same average nucleotide lengths, ligand, and LDs, the assay SBNR values for all, or nearly all, particular gene comparisons should be essentially the same. For a cell sample L-LPN prep comparison assay, different particular gene LPN comparisons in the assay can be associated with different L-LPN nucleotide lengths and LDs, and therefore different SBNR values. This indicates that the SBNR UNF is a non-global assay variable UNF. Earlier sections described the determination of the nucleotide lengths of particular gene LPNs by measurement and inference. Such methods apply directly to the determination of the nucleotide lengths of particular gene L-LPN molecules in cell sample L-LPN preps. Further, earlier sections also described the determination by measurement and inference, of the label densities associated with particular gene LPN molecules in a cell sample LPN prep. Such methods apply directly to the determination of particular gene L-LPN molecules in cell sample L-LPN preps.

The above discussion applies directly to assays which compare Type 1 L-LPN molecules, except in cases where the SGC molecule used in the assay is similar to or greater in molecular size than the immobilized Type 1 L-LPN molecule.

Exogenous standard DNA molecules can be used to determine the SBNR value for a cell sample L-LPN prep comparison assay which is associated with a specific combination of compared L-LPN nucleotide lengths and LDs, and uses only one ligand and one SGC molecule type in the assay. For this cell sample L-LPN comparison assay, the nucleotide lengths and LDs of all particular gene L-LPN molecules in a compared L-LPN prep are the same, while the nucleotide lengths and LDs of the compared L-LPN preps are not the same. The S method for determining the assay SBNR values follows. (a) Produce a preparation of S Type 1 L-LPN molecules which has the same nucleotide length and LD as one cell sample L-LPN prep. Produce another preparation of a different S Type 1 L-LPN molecules which has the same nucleotide length and LD as the other compared cell sample L-LPN prep. Each S L-LPN prep is associated with the same ligand. Each S L-LPN prep has a similar base composition and has a minimum of intrastrand secondary structure. In addition, each S L-LPN prep is produced so that the same known number of a particular fluorescent dye molecule is associated with the 3′ or 5′ end of each S L-LPN molecule. Such dye molecules should be readily detected and quantitated in the presence of a SGC molecule. Such dye molecules can be used to normalize for differences in hybridization rates caused by the different nucleotide lengths. (b) Add to one hybridization solution equal amounts of each different S L-LPN prep, and the amount used should give a strong but far from saturating signal. (c) The hybridization mix is incubated with an array which contains replicate spots for each different S L-LPN, and each spot is specific for only one S L-LPN type. Preferably such replicate spots should be made in such a way so that the print tip and print place CNF values are equal to one, or are not pertinent for the assay. Such S spots should be located on each array in multiple locations and in the proper number in order to obtain a statistically significant sampling of the array spatial surface. (d) After hybridization and post-hybridization washing and processing, the array is stained with a solution of one SGC type for a long enough time to ensure maximum binding of the SGC molecules to the hybridization immobilized ligand molecules. After the post-stain wash and processing the array is ready for the signal generation and detection step. At this point, each replicate shorter S L-LPN spot should all contain the same number of hybridized L-LPN molecules, ligand molecules, and dye molecules. Similarly, each replicate longer S L-LPN spot should contain the same number of hybridized L-LPN molecules, ligand molecules, and dye molecules. The number of hybridized L-LPN molecules and dye molecules is likely to be higher in each shorter L-LPN spot relative to each longer L-LPN spot, because the shorter L-LPN molecules are reported to hybridize faster than longer L-LPN molecules to immobilized CDP molecules. At this point the relative number of SGC molecules immobilized in each S spot is not known and must be determined. (e) For each replicate S spot, the signal activity associated with the dye, and the SGC, is measured using the same measurement conditions for each spot on the array. The SGC RAS value and the dye RAS value is determined for each spot. For each replicate longer L-LPN S spot, the dye RAS value should be the same for each spot, and the SGC RAS value should be the same for each spot. Similarly, for each replicate shorter L-LPN S spot, the dye RAS value should be the same for each spot, and the SGC RAS value should be the same for each spot. In order to control for possible spatial surface difference effects on the RAS values, the average dye and SGC RAS value for all S replicates is determined for the longer S spots and the shorter S spots. Here, it is reasonable to assume that the SSAR=1 for each S spot replicate. (f) The ratio of (the average SGC RAS value per spot for the longer S L-LPN replicate spots)÷(the average SGC RAS value per spot for the shorter S L-LPN replicate spots), is termed the average SGC RAS ratio or average SGC ratio, or ASR. (g) The ratio of (the average dye RAS value for the longer S L-LPN replicate spots)÷(the average dye RAS value for the shorter S L-LPN replicate spots), is here termed the Average Dye Ratio, or ADR. The ADR reflects the number of longer S L-LPN molecules hybridized to the average longer S L-LPN spot, relative to the number of shorter S L-LPN hybridized to the average shorter S L-LPN spot. The ADR can be used to normalize the ASR value so that it reflects a comparison of equal numbers of hybridized shorter and longer S L-LPN molecules. (h) For this short vs. long S L-LPN comparison the (SBNR)=(ASR)÷(ADR). (i) For any particular gene L-LPN comparison of similar short and long L-LPN molecules (the S derived SBNR)=(the particular gene L-LPN comparison SBNR).

Note that such exogenous S L-LPN molecules can be incorporated into a cell sample L-LPN prep comparison assay in order to determine the SBNR value for the assay. Note also that one or more nucleotide length or LD difference effects on the assay SBNR values can be determined in the same assay by utilizing multiple different S L-LPN combinations. From such multiple combination results, a standard curve can be created. Note further that the same assay reagents and procedures should be used for generating the standard curve results, and the cell comparison assay results associated with the standard curve.

The normalization of the L-LPN assay measured particular gene RASR value for the particular gene SBNR value is straightforward and, (the SBNR normalized RASR)=(the RASR)÷(SBNR). Prior art does not determine or take into consideration during the normalization step, the SBNR.

The SBNR determination and the SBNR normalization process can be greatly simplified by designing the assay so that the SBNR for the assay acts as a global UNF. This can be done by designing the assay so that all particular gene comparisons in the assay involve the comparison of L-LPNs which have the same nucleotide length and LD values. In such a situation the assay SBNR value associated with each particular gene L-LPN comparison in the assay is equal to one, and can be ignored. Alternatively, the simplification can involve the comparison by design of particular gene L-LPN molecules which have different nucleotide lengths and different LD values, and the nucleotide length and LD differences are the same for each particular gene L-LPN comparison in the assay. In this situation the SBNR value associated with each particular gene L-LPN comparison in the assay is the same, but is not equal to one. Methods for producing compared L-LPN molecules which have the same nucleotide lengths and LDs were discussed earlier.

Note that while the above discussion focused primarily on SGDS particular gene mRNA transcript L-LPN comparisons, the discussion also applies to SGDS, DGDS, and DGSS particular gene RNA transcript of all kinds L-LPN comparisons.

One of skill in the art will recognize al ternate methods for determining SBNR values.

SSAR Determination and Normalization.

The UNF SSAR is pertinent only to cell sample Type 1 indirect label L-LPN comparisons assays. The SSA is expressed in terms of the signal activity associated with one L-LPN immobilized SGC molecule. It is necessary to know the SSA values which are associated with particular gene L-LPN comparisons in order to know whether the assay measured particular gene RASR value requires normalization for the SSAR. Most prior art indirect label L-LPN comparison assays involve Type 1 L-LPNs which utilize only one ligand type and one SGC type for the assay. For these prior art assays each compared particular gene spot is stained with identical SGC stain solutions made from one SGC stock solution, and the staining conditions and signal measurement conditions are identical for each compared spot. Here, only the spot surfaces are different, and prior art believes and practices that differences associated with the compared array surfaces do not significantly affect the assay SSA values. For such an assay it is reasonable to assume, as the prior art does, that the SSAR=1 for all particular gene L-LPN comparisons, and that the SSAR can be ignored for normalization.

For a cell sample Type 1 indirect label L-LPN comparison which associates a different ligand•SGC combination with each compared cell sample L-LPN, that is a two label assay, it cannot be assumed that the SSAR is equal to one for all particular gene comparisons. This occurs because different SGC molecule types are generally associated with different SSA values and the assay SSAR value does not equal one. The SSAR for such a situation can be determined and is equal to the ratio of (the measured signal activity associated with a known volume and molarity of one SGC type)÷(the measured signal activity associated with the same known volume and molarity of the second SGC type). Further, the SSA value for each different type should be measured under the same signal generation and detection conditions which are used to obtain the cell sample L-LPN comparison signal activity results. For a particular gene comparison assay measured RASR value, (the SSAR normalized RASR)=(measured RASR)÷(SSAR).

One of skill in the art will recognize that there are a variety of alternate methods for determining the SSAR for a cell sample L-LPN comparison.

Normalization of Particular Gene Comparison Assay Measured Results for Unconsidered Assay Variables (UNFs).

Unconsidered global and non-global assay variables are associated with the unconsidered assay variable normalization factors or UNFs, SCR, PAFR, MLDR, PL-HKR, PS-HKR, PSAR, PSSR, LLSR, SBNR, and SSAR. These UNFs can be used to normalize or correct particular gene comparison assay measured results for prior art unconsidered assay variables. The UNFs which may be pertinent to a direct label Type 1 cell sample LPN comparison are the SCR, PAFR, MLDR, PL-HKR, PS-HKR, PSAR, and PSSR. Those UNFs which may be pertinent to an indirect label Type 1 L-LPN comparison are the SCR, PAFR, MLDR, PL-HKR, PS-HKR, SBNR, and SSAR. Those UNFs which may be pertinent to a direct label Type 2 LPN comparison are the SCR, PAFR, PL-HKR, PS-HKR, and LLSR. Those UNFs which may be pertinent to an indirect label Type 2 L-LPN comparison are the SCR, PAFR, PL-HKR, PS-HKR, SBNR, and LLSR.

For a particular gene comparison, a pertinent assay NF or UNF or CNF, is associated with an assay variable or variables which can cause the particular gene assay measured RASR value to deviate from the assay ACR value for the particular gene, or which can cause the particular gene ACR value to deviate from the T-DGER value for the particular gene. When a pertinent NF or CNF or UNF does not equal one for a particular gene comparison, then the particular gene RASR value must be normalized for the pertinent NF or CNF or UNF, unless the value for the UNF is compensated for by a different pertinent assay variable NF or CNF or UNF assay value, for the particular gene. When a pertinent NF or CNF or UNF assay value equals one, the particular gene RASR does not require normalization for the pertinent NF or CNF or UNF.

The process of normalizing a particular gene LPN comparison assay RASR result with the UNFs involves dividing the particular gene comparison assay RASR result by the assay value for each of the UNFs which are pertinent to the assay, or by the product of the pertinent assay UNF values. Herein the product of the pertinent UNF assay values is termed the UNF product, or UNFP. The result of this normalization is an NASR value for the particular gene LPN comparison, which is normalized for the pertinent UNFP. For the particular gene LPN comparison then, the (UNF normalized NASR)=(assay RASR). (UNFP). As discussed earlier, it is assumed that a particular gene comparison RASR value has been correctly adjusted for assay background, and the assay variables associated with assay background, and its measurement.

Normalization of Particular Gene Expression Comparison Assay Results for Prior Art Considered Assay Variables.

The primary global and non-global assay variable NFs which are considered for prior art normalization of gene expression comparison assay results, are the ARR, C-HKR, ALDR or TSAR, spatial, print tip, print plate, intensity, scale, image analysis associated, background associated, random noise, and AE•AE NFs. Prior art considered NFs are herein termed CNFs. The scale CNF refers to both within slide and between slide scale normalization.

A microarray gene expression comparison assay particular gene NASR or N-DGER value is derived from the assay measured quantitative signal activity associated with each cell sample's particular gene LPN which is hybridized to the particular genes microarray spot. The total signal activity present in the spot, the TSS, is determined for the particular gene. Before further normalization, prior art almost always adjusts each TSS for assay background signal and for image analysis, and in some cases for non-specific hybridization, thereby producing a raw assay signal or RAS value, for each compared particular gene. The particular gene RASR value can then be determined from the RAS values for each compared particular gene. Established prior art methods are used to adjust the TSS for background signal, image analysis, and non-specific hybridization (7, 34, 35). Prior art generally regards non-specific hybridization as being associated with the background. Such adjustments produce a particular gene RAS value. For an assay each particular gene RAS value can be normalized for each of the pertinent prior art considered assay variables, thereby producing a particular gene NAS value for each cell sample's particular gene. Alternatively, the particular gene comparison RASR value can be determined and normalized for each pertinent CNF value, thereby producing a particular gene comparison NASR value. For simplicity the normalization of the particular gene comparison RASR values with the assay pertinent CNFs is discussed. Prior art CNFs are expressed in terms of the ratio of, (the assay variable quantitative value for the particular gene of one cell sample)÷(the assay variable quantitative value for the same particular gene of another compared cell sample). Prior art CNFs which can be pertinent to the prior art microarray assay normalization of a particular gene RASR value are the ARR, C-HKR, TSAR, spatial, print tip, print plate, intensity, and scale CNFs (7, 31, 33, 34, 35, 37).

For a particular gene comparison, a pertinent assay NF, CNF, or UNF, is associated with an assay variable or variables which can cause the particular gene assay measured RASR value to deviate from the assay ACR value for the particular gene, or which can cause the particular gene ACR value to deviate from the T-DGER value for the particular gene. When a pertinent CNF≠1 for a particular gene comparison, then the particular gene RASR value must be normalized for the pertinent CNF, unless the CNF is compensated for by a different particular assay variable CNF or UNF assay value. When the pertinent CNF 1, the assay measured particular gene RASR value does not require normalization for the CNF.

Each of the individual CNFs has an essentially independent effect on the biological accuracy of an assay measured particular gene RASR value. The aggregate effect of the CNFs which are pertinent to an assay, and which have assay values not equal to one, is the product of all these pertinent CNFs. Herein, this is termed the assay CNF product, or CNFP. A particular gene RASR value requires normalization for one or more CNFs when for that particular gene RASR value, the assay CNFP≠1.

Normalization of a particular gene RASR value for the ARR, TSAR, C-HKR, spatial, print tip, print plate, intensity, scale, and AE•AE CNFs which are pertinent to an assay, can be done using the relationship, (normalized RASR)=(NASR)=(RASR)÷(CNFP). Normalization of a particular gene RASR for a single CNF can be done using the relationship, (normalized RASR)=(RASR)÷(CNF). Normalization of random noise CNF can be done with well established procedures.

Prior art generally regards the CNFs ARR, C-HKR, and TSAR, to be associated with global assay variables, and the spatial, print tip, print plate, intensity, scale, and AE•AE CNFs to be associated with non-global assay variables. As discussed, the TSAR is, in reality, associated with both global and non-global assay variables. The dye swap method is claimed by the prior art to normalize each particular gene comparison RASR for TSAR or ALDR related non-global assay variables associated with differences in the intrinsic signal activities of different and the same dyes, differences in the incorporation efficiencies of different and the same dyes into compared cell sample LPN preps, and differences in the signal detection efficiencies of the compared dyes (7,160). The dye swap method will effectively normalize for these dye related differences only under certain specific assay conditions, which often do not occur. Further, prior art does not determine whether the assay conditions are appropriate for valid dye swap normalization or not. Therefore for those prior art assays which use the dye swap method for normalization, it cannot be known whether the dye swap normalization is valid or not, with regard to the accurate normalization for dye related differences in the assay. Note that the dye swap method does not normalize for TSAR related differences in the compared LPN signal activities caused by RNA purity related differences in dye incorporation into compared LPNs, or assay signal activity differences related to differences in nucleotide length of the compared LPNs. Therefore, a prior art dye swap normalized particular gene NASR or N-DGER cannot be known to be completely normalized for all non-global variable aspects of the TSAR, absent further information. Note further, that relatively few prior art assays use the dye swap method for normalization of dye related global and non-global assay variables.

Prior art has developed, and practices, a variety of different approaches for the normalization of particular gene comparison assay measured RASR values for CNFs (7, 34, 35, 37). Prior art microarray practice rarely directly determines the assay values for each pertinent assay CNF. Instead, these prior art normalization approaches generally assume the validity of certain key assumptions which, if valid, allows the normalization for the prior art regarded global CNFs ARR, C-HKR, and TSAR, without determining the assay value for each pertinent global CNF. These key normalization assumptions, if valid, also allow for the separate determination of each of the non-global CNF values for each of the non-global CNFs spatial, print tip, print plate, and intensity. These values are then used for normalization of the particular gene comparison results. For within slide and between slide normalization of the distribution of otherwise normalized particular gene NASR values, a further scale normalization is sometimes used. The validity of such a scale normalization is also dependent on the validity of the key prior art normalization assumptions. The within slide and between slide scale NFs are non-global NFs.

As discussed in another section these key prior art normalization assumptions can be known to be invalid for certain prior art microarray and non-microarray assays and are probably invalid for many others, and cannot be known to be valid for most prior art microarray and non-microarray assays. Consequently, for the large majority of prior art microarray and non-microarray assay gene comparison NASR or N-DGER values, it cannot be known whether a particular gene NASR value is correctly normalized or not. The CNFs which are affected by the validity of the key prior art normalization assumptions are the ARR, C-HKR, TSAR, spatial, print tip, print plate, intensity, and scale CNFs. As a result, when these key prior art normalization assumptions are known to be invalid, or cannot be known to be valid or invalid, an alternate, improved method of CNF normalization which does not rely on the validity of these key prior art normalization assumptions, is required. Such a method is described below.

Certain prior art normalization methods involve the incorporation of one or more exogenous standard (S) mRNA LPNs into each compared cell sample LPN prep, as well as the incorporation of one or more CDP spots specific for each different S, on the microarray surface. These S molecules may consist of either RNA or DNA. Such S molecules are claimed by the prior art to provide the basis for the prior art normalization of the global assay variables associated with differences in compared cell sample LPN prep labeling, differences in compared cell sample LPN hybridization kinetics, differences in the signal activities of different LPN label types, and differences in the amounts of RNA compared.

Absent the known validity of the prior art key normalization assumptions, exogenous S molecules can provide the basis for the prior art normalization of the spatial, print tip, print plate, intensity, and scale CNFs. This can be accomplished using one or more S assay designs. One such S assay design can involve adding labeled or unlabeled S mRNAs to the compared cell samples labeled or unlabeled RNA or cRNA. Alternatively the design may involve adding labeled or unlabeled S DNA to the cell sample's cDNA preps. Both of these approaches may be used in the same assay.

One design involves the incorporation of known amounts of the same S LPN type into each compared cell sample LPN, and the inclusion of numerous replicate CDP spots for the S into the microarray slide. The known amount of S LPN added to each cell sample LPN may be known equal amounts, or known unequal amounts. Here, replicate CDP spots appropriately spaced over the entire slide are required. This design can be used to normalize for the global ARR and C-HKR CNFs, and the non-global spatial, print tip, print plate, and scale, CNFs. For this same cell sample LPN comparison assay, the S method can also be used to normalize for the intensity CNF. This involves the following. Incorporate into each cell sample LPN a known but different amount for each of multiple different exogenous S LPN molecules, and incorporate into the microarray one or preferably multiple replicate CDP spots for each different exogenous S. For each individual S LPN equal or unequal, but known, amounts are incorporated into each compared cell sample LPN prep. Here, the known but different amounts of the different S LPN molecules incorporated into each cell sample LPN prep, should span a concentration range which is adequate for the purpose of normalizing for the different particular gene signal intensity values obtained in an assay. Here, replicate spots which are appropriately spaced over the microarray surface are preferred.

An alternative S assay design is to incorporate known equal or unequal amounts of one or more S mRNA types into each cell sample T-RNA or mRNA prep, and the inclusion into the microarray of one or replicate CDP spots for each different S mRNA type. Generally the addition of known equal amounts of each S mRNA type is preferred. Here, replicate spots appropriately spaced over the entire slide are required. This design can be used to normalize for the global ARR and C-HKR CNFs, and for the global component of the TSAR CNF, as well as for the non-global spatial, print tip, print plate, and scale CNFs. For this same cell sample LPN comparison assay, the S design can also be used to normalize for the intensity CNF. This involves the following. The incorporation into each cell sample T-RNA or mRNA of a known but different amount for each of the multiple different S mRNA molecules, and incorporate into the microarray one or replicate CDP spots for each different exogenous S mRNA type. For each individual S mRNA, equal or unequal but known amounts of S mRNA are incorporated into each compared cell sample T-RNA or mRNA prep. Here, the known but different amounts of the different S mRNAs incorporated into each cell sample T-RNA or mRNA prep, should span a concentration range which is adequate for the purpose of normalizing for the different particular gene signal intensity values associated with the assay results. Here, replicate spots which are appropriately spaced over the microarray surface are preferred.

In order to normalize for the global CNFs ARR and C-HKR using the S method, it is valid to use the same prior art normalization methods which were used in conjunction with the key prior art assumptions. For the S method, each S gene or mRNA type in the assay can be known to have a known S ACR value which is equal to one. A prior art key assumption assumes, but does not know, that the cell sample genes used for normalization have a T-DGER equal to one. Because each S replicate, and each different S in the assay has an S ACR value known to equal one, the prior art methods which require such a condition can be used to normalize for the global CNFs ARR, and C-HKR, and the global aspects of the TSAR, as well as the non-global CNFs spatial, print tip, print plate, intensity, and scale. Such prior art methods include, but are not limited to, scatterplots or various kinds, and global and local regression analysis (7, 34, 35). When normalizing for just the CNFs, an appropriate order of normalization is: First, the global CNFs ARR and the global aspects of the TSAR; second, the spatial, print tip, print plate, and intensity non-global CNFs; third, the scale CNF. These prior art methods of normalization using the S method do not require the direct determination of each assay CNF value for each particular gene comparison in the assay. For example, the prior art method of normalizing for the global CNFs ARR, and C-HKR, and for the global component of the TSAR, normalizes for all three simultaneously with a composite CNF value which includes the assay values of all three of these CNFs. Here, if the ARR and/or C-HKR and/or TSAR CNF assay value consists of both a global and non-global component, then the prior art normalization process will result in an incompletely normalized NASR value for most particular gene comparisons in the assay. To complete the normalization of each of these particular gene NASR values, a direct or indirect measure of the assay values for the non-global assay value or values must be known.

A variety of different S method designs for effective normalization is possible. Note the S method normalization for the CNFs cannot be used to normalize for assay variables associated with the following. (i) The intrinsic biological aspects of the compared cell sample RNAs. These include the T-RNA or mRNA content per cell, the number of cells from each cell sample which are compared in the assay, and the nucleotide length, sequence and composition of the compared particular gene mRNAs. (ii) The nucleotide length, nucleotide sequence, or nucleotide composition of compared cell sample particular gene LPNs. (iii) The non-global components associated with LPN labeling, LPN signal detection, and label differences.

Prior art normalization of Northern Blot (NB), Dot Blot (DB), Nuclease Protection (NP), and RT-PCR, particular gene comparison assay results generally do not require the validity of the key normalization assumptions which are required for prior art microarray normalization methods. Generally, for DB, NB, and NP assays: only one particular gene mRNA is assayed for; only one radioactive LPN type is used for each assay; the C-HKR and TSAR CNFs do not have to be normalized for; the cell sample T-RNAs or mRNAs are directly compared. As a result most of the CNFs which are pertinent for microarray assays, are not pertinent for the DB, NB, or NP assays. As an example, for a particular prior art gene comparison NP assay, only the ARR CNF is considered for normalization. Similarly, for prior art RT-PCR assays the CNF ARR and AE•AER and AE-SER are pertinent to the assay. Note that for the microarray, DB, NB, NP, and RT-PCR assays, the CNF ARR is incorporated into the assay value for the UNF SCR. Here, normalizing for the UNF SCR will also normalize for the CNF ARR. However, normalizing for the ARR does not normalize for the SCR.

Note that on very rare occasions, prior art gene expression analysis practice identifies as an assay variable the cell sample cDNA yield fraction.

The process of normalizing a particular gene comparison assay RASR result with the CNFs, involves dividing the particular gene comparison assay RASR result by the assay value for each of the CNFs which are pertinent to the assay, or by the product of the pertinent assay CNF values. Herein the product of the pertinent CNF assay values is termed the CNF product, or CNFP. The result of this normalization is an NASR value for the particular gene comparison, which is normalized for the pertinent CNFP. For the particular gene LPN comparison then, the (CNFP normalized NASR)=(assay RASR)÷(CNFP). As discussed earlier, it is assumed that a particular gene comparison RASR value has been correctly adjusted for assay background, and the assay variables associated with assay background, and its measurement.

Normalization of Particular Gene Comparison Assay Results for CNFs and UNFs.

The complete and accurate normalization of microarray and non-microarray gene expression comparison assay measured particular gene RASR values, will produce particular gene NASR or N-DGER values which equal the particular gene T-DGER, and are therefore biologically accurate. In order to be completely and accurately normalized, such particular gene NASR values must be accurately normalized for all assay pertinent NFs, including all pertinent CNFs and UNFs. Such pertinent CNFs or UNFs can cause an assay measured particular gene RASR value to deviate significantly from biological accuracy. Prior art microarray and non-microarray practice does not determine or normalize for pertinent UNF values. Prior art microarray and non-microarray practice produces many particular gene NASR values which are normalized for pertinent CNFs, and which cannot be interpreted with regard to the completeness of normalization since, absent further knowledge which is not provided by the prior art, it cannot be known whether these prior art particular gene NASR values require further normalization for pertinent UNFs or not. Prior art microarray and non-microarray practice also produces particular gene NASR values which can be known to be incompletely normalized, and require further normalization for one or more pertinent UNFs. No prior art microarray or non-microarray practice produced particular gene NASR values are known to be completely normalized for pertinent UNFs. As a result of all this, all prior art microarray and non-microarray produced particular gene NASR values are either known to be incompletely normalized, and therefore biologically inaccurate, or are uninterpretable with regard to completeness of normalization for UNFs and biological accuracy. In addition to the above, the prior art produced particular gene NASR values which are normalized for CNFs, cannot be known to be validly or accurately normalized for the pertinent CNFs, because the prior art normalization process used to produce these NASR values cannot be known to be valid. This occurs for virtually all prior art microarray produced NASR values, and for many non-microarray NASR values. Improved methods for the accurate normalization of particular gene RASR values for pertinent CNFs were discussed in the section on normalization for CNFs. Because of the above considerations, all or almost all prior art microarray produced particular gene NASR values are either: (a) known to be incompletely normalized for UNFs, and uninterpretable with regard to the accuracy and validity of the normalization for CNFs, or; (b) uninterpretable with regard to the completeness of normalization for UNFs, and uninterpretable with regard to the accuracy and validity of the normalization for CNFs. Similarly, all or almost all prior art non-microarray produced particular gene NASR values are either known to be incompletely normalized for UNFs, or are uninterpretable with regard to the completeness of normalization for UNFs. Many prior art non-microarray produced NASR values are uninterpretable with regard to the accuracy and validity of the normalization process for the CNFs.

Relative to prior art microarray and non-microarray particular gene NASR values which are produced by normalizing for CNFs using the prior art normalization process, the normalization of microarray and non-microarray measured particular gene RASR values for pertinent CNFs and UNFs using a valid normalization process, produces improved particular gene NASR values which are: (a) known to be validly and accurately normalized for pertinent CNFs and UNFs; (b) known to be more completely normalized and more biologically accurate; (c) known to be more interpretable. Overall then, such particular gene NASR values are known to be more accurate, interpretable, reproducible, intercomparable, reproducible, and have greater utility.

It is believed that the CNFs and UNFs described here represent the microarray and non-microarray gene expression analysis associated assay variables which commonly occur for these assays, and which can cause an assay measured particular gene RASR value to deviate significantly from the particular gene comparison assay values for T-DGER or ACR, or both. Other potential assay variable factors exist which may cause a particular gene RASR value to deviate significantly from assay or biological accuracy. These are summarized below, and include but are not limited to, the following. (a) Second strand cDNA synthesis in the first strand cDNA synthesis and/or labeling step. (b) Non-specific hybridization of the LPN to the wrong particular gene spot. (c) The presence of antisense RNA for a particular gene RNA transcript. (d) The effect of ozone on the assay signals. (e) The presence of splicing variants for mRNAs. (f) The presence of non-specific cRNA or cDNA in the cell sample cRNA or cDNA prep. (g) The linearity of the input RNA versus the observed assay signal for particular gene RNAs. (h) The accurate quantitation of the cell sample nucleic acids associated with an assay.

This discussion concerns the normalization of microarray and non-microarray assay measured particular gene RASR values for all pertinent CNFs and UNFs. A particular gene NASR value which is completely normalized for the pertinent CNF and UNFs is equal to, (the particular gene RASR value)÷(product of the pertinent CNF values×the product of the pertinent UNF values). In other words, (the particular gene NASR value)=(particular gene RASR value)÷(pertinent CNFP value×pertinent UNFP value). The (pertinent CNFP)×(pertinent UNFP) value is termed the assay pertinent NFP value, or simply the PNFP value. Here then, (particular gene NASR)=(particular gene RASR)÷(particular gene PNFP).

The CNFs and UNFs which may be pertinent to a microarray or non-microarray assay, are the C-HKR, spatial, print tip, print plate, intensity, scale, and AE-SER and AE•AER CNFs, and the SCR, PAFR, MLDR, PL-HKR, PS-HKR, PSAR, PSSR, LLSR, SBNR, and SSAR UNFs. Note that the global CNF ARR assay value is incorporated into the global UNF SCR assay value, but is only one component which contributes to the SCR value. Thus, the ARR value is not used for normalization, but the SCR value is. Note further that as discussed earlier, the CNF TSAR value almost always has both a global and non-global component, and represents a complex average measure of all of the cell samples particular gene PSAR values. Thus, the TSAR value for a cell sample comparison cannot be used to accurately normalize all assay particular gene RASR values, and is not an appropriate NF for accurate normalization. The global NFs which may be pertinent to a microarray or non-microarray assay are represented by the CNF C-HKR, and the UNFs SCR, LLSR, and SSAR. The NFs which are associated with non-global assay variables and which may be pertinent, are represented by the CNFs spatial, print tip, print plate, intensity, scale, and AE•AER, and the UNFs, PAFR, MLDR, PL-HKR, PS-HKR, PSAR, PSSR, and SBNR.

The process of normalizing for the assay pertinent NFs varies in difficulty and complexity, depending on the method of gene expression comparison used. Microarray gene expression comparison assays which compare cell sample directly or indirectly fluorescent labeled LPN preps, require a complex normalization process. Many prior art microarray particular gene comparisons employ directly labeled fluorescent LPNs. Microarray assays which compare radioactive labeled cell sample LPNs also requires a complex normalization process which is generally associated with more readily determined assay NF values, than the microarray fluorescent LPN assays. Dot Blot (DB), Northern Blot (NB), and Nuclease Protection (NP) assays, require a relatively simple normalization process, which is associated with a much smaller number of NFs than the microarray assays. RT-PCR assays appear to be associated with fewer NFs than microarrays, but may be more difficult to normalize than microarray assays because of the variability of the RT-PCR assay AE-SER and AE•AER values. A description of a possible normalization process or processes for each of these different methods, is presented below.

Many prior art microarray measured particular gene RASR values are associated with directly labeled fluorescent LPNs, and a lesser fraction with radioactive LPNs. For such microarray assays, the NFs which may be pertinent are the global NFs C-HKR, SCR, and LLSR, and the non-global NFs spatial, print tip, print plate, intensity, scale, PAFR, MLDR, PL-HKR, PS-HKR, PSAR, and PSSR. The vast majority of the microarray directly labeled radioactive and fluorescent LPN assay comparisons, are associated with Type 1 LPNs. For these microarray assays the NFs which may be pertinent are the global NFs C-HKR, and SCR, and the non-global NFs spatial, print tip, print plate, intensity, scale, PAFR, MLDR, PL-HKR, PS-HKR, PSAR, and PSSR. A small fraction of microarray assays are associated with radioactive or fluorescent direct label Type 2 LPNs. Here the NFs which may be pertinent are the global NFs SCR, C-HKR and LLSR, and the non-global NFs spatial, print tip, print plate, intensity, scale, PAFR, PL-HKR, PS-HKR, and rarely PSSR.

An example of the normalization of particular gene RASR values produced by microarray assays associated with compared cell sample directly labeled radioactive or fluorescent Type 1 LPN preps will be described first. The prior art normalization process for such particular gene RASR values generally involves first normalizing for pertinent global CNFs, and then, in a stepwise fashion, normalizing for the pertinent non-global CNFs. Normalization for the scale CNF is generally done last. A similar approach can be used for NF normalization. This NF normalization process follows. (i) First normalize each particular gene RASR value in the assay for the pertinent global NFs. For microarray assays which compare cell sample fluorescent or radioactive Type 1 or Type 2 cell sample LPNs in one hybridization solution, the global NF C-HKR can be ignored. For such assays which use only one label, the C-HKR is not ignored. (ii) Then normalize each particular gene partially normalized NASR value for each of the non-global NFs PAFR, MLDR, PL-HKR, PS-HKR, PSAR, and PSSR, which is pertinent. For those microarray assays which compare cell sample LPNs produced from T-RNAs by random priming, the PAFR is not a pertinent NF. (iii) Then normalize each particular gene partially normalized NASR value from ii for the NFs spatial, print tip, print plate, and intensity, which are pertinent. Note that the print tip normalization is often used for the normalization of general spatial variation across a microarray. Well established methods are available to accomplish the normalization for each of these CNFs. (iv) Then do within, and between slide scale normalization for the distribution of the particular gene NASR values from iii. Scale normalization can be omitted in the event the distributions are reasonably consistent.

Only a small fraction of prior art microarray gene expression comparison assays compare fluorescent or radioactive Type 2 LPN preps. For such microarray assays, the NFs MLDR and PSAR are not pertinent, and in effect the PSSR is pertinent only rarely. The normalization process for such radioactive or fluorescent Type 2 microarray assays can be essentially the same as described above for the microarray radioactive or fluorescent Type 1 LPN associated assays. Note that, as with the Type 1 assays, the C-HKR and PAFR NFs are pertinent only under certain conditions.

The above described normalization process for microarray assay results, is believed to be appropriate and adequate for most if not all microarray assay situations. However, the described process can be validly modified in a variety of ways with regard to the order of normalization for the various pertinent NFs, and the type and form of the NF used for normalization. The form of the NF refers to whether a particular NF is associated with other NFs to create a composite NF value, which is then used for the normalization.

Accurate and valid normalization for all pertinent NFs, including all pertinent CNFs and UNFs, is necessary in order to produce microarray assay particular gene NASR values which are maximally improved in completeness of normalization and biological accuracy, relative to prior art produced microarray assay particular gene NASR values. In order to obtain such microarray assay produced improved particular gene NASR values, it is necessary to improve the prior art normalization process as follows. (i) It is necessary to use an improved approach for the determination of pertinent CNF values and the normalization of particular gene RASR values for these pertinent CNFs, which can be known to be valid and does not rely on assuming the validity of the key prior art normalization assumptions. As discussed earlier, it can be known for certain prior art microarray assays that the assumptions which are key to the determination of the CNFs, and normalization for the CNFs, are invalid. Further, it is likely that these assumptions are invalid for many, and possibly most, prior art microarray assays. As a result, absent information which is not available in the prior art, it cannot be known whether the assumptions are valid for any specific microarray assay or not. Therefore, in order to know that the pertinent CNF values for an assay are validly and accurately normalized, it is necessary to improve the process of determining the pertinent CNF values and normalizing for them. Such an improved CNF determination and normalization process which utilizes the use of S molecules was described in the section on normalization for CNF values. Alternatively, such an improved CNF determination and normalization process can involve determining that the prior art key assumptions for normalization are valid, and then using standard prior art methods for normalization. (ii) It is necessary to use an improved overall normalization process for microarray assay measured particular gene RASR values which includes the identification of pertinent assay UNFs, the valid and accurate determination of pertinent UNF values, and the valid and accurate normalization for the UNFs values, as well as the valid and accurate determination of CNF values, and valid and accurate normalization for the CNF values. Note that for those microarray assays where the key prior art normalization assumptions are determined to be valid, the improved UNF normalization process is used in combination with the prior art method for normalization of the pertinent CNFs and the knowledge that the prior art key normalization assumptions are valid, to produce the improved particular gene NASR values.

For DB, NB, and NP gene expression comparison assay produced particular gene RASR values, the number of pertinent NFs associated with each assay can be far smaller than for microarray assays. Here, the pertinent NFs are generally the global SCR and the non-global PAFR, and when the DB, NB, or NP assay compares cell sample T-RNAs, the PAFR is not a pertinent NF. Here, (the particular gene RASR value)÷(pertinent NFP value)=(particular gene NASR value). Certain DB, NB, or NP assay designs will involve additional pertinent NFs.

For RT-PCR gene expression comparison assay produced particular gene RASR values, the global UNF SCR and non-global UNF PAFR are pertinent UNFs, and when the RT-PCR assay compares cell sample specific gene or random primed T-RNA cDNA preps, only the SCR is a pertinent UNF. As discussed, the pertinent CNFs AE•SER and AE•AER, as well as other prior art known assay variables also must be adequately controlled and normalized for. Here, (the particular gene RASR value)÷(the pertinent NFP value)=(particular gene NASR value).

Accurate and valid normalization for all pertinent NFs, including all pertinent CNFs and UNFs, is necessary in order to produce non-microarray assay produced particular gene NASR values which are maximally improved in completeness of normalization and biological accuracy, relative to prior art non-microarray assay produced particular gene NASR values. In order to obtain such non-microarray assay produced improved particular gene NASR values, it is necessary to improve the prior art normalization process as follows.
(i) Improve the prior art normalization process for pertinent CNFs by not normalizing directly for the CNF ARR. The global CNF ARR is a component of the UNF SCR, and will be normalized for when the SCR is normalized for. (ii) Improve the overall non-microarray normalization process by including the accurate identification of pertinent NFs, and the accurate determination of pertinent CNF and UNF values, and the valid and accurate normalization for these pertinent CNF and UNF values.

A microarray assay measured particular gene NASR value which is validity, completely, and accurately normalized for all pertinent NFs, is biologically accurate within the limits of the measurement accuracy of the microarray assay. Therefore, such completely normalized particular gene NASR values from different microarray assays and different microarray platforms are directly and validly intercomparable within the measurement accuracy limits of the microarray assays. Similarly, the microarray assay measured NASR values for different particular genes in the same microarray assay, or NASR values for different particular genes in different microarray assays, are directly and validly intercomparable, within the measurement accuracy limits of the microarray assay or assays.

A non-microarray assay measured particular gene NASR value which is validly, completely, and accurately normalized for all pertinent NFs, is biologically accurate within the measurement accuracy limits of the non-microarray assay. Therefore the same particular gene NASR values obtained from different non-microarray assays of the same and different non-microarray method type, are directly and validly intercomparable within the measurement accuracy limits of the non-microarray assays. Similarly, the non-microarray assay measured NASR values for different particular genes in the same non-microarray assay, or NASR values for different particular genes in different non-microarray assays, are directly and validly intercomparable, within the measurement accuracy limits of the non-microarray assay, or different non-microarray assays of the same or different type.

Similarly, when properly normalized, microarray and non-microarray assay measured particular gene NASR values for the same particular gene, and different particular genes, are directly and validly intercomparable within the measurement accuracy limits of the compared microarray and non-microarray assays.

NFs which are commonly pertinent for many microarray and non-microarray gene expression comparison assays are described here and taken into account during the improved normalization process. This improved normalization process is necessary for producing microarray and non-microarray particular gene NASR and N-DGER values which are biologically accurate. However, even in the event that not all of the pertinent NFs have been identified and normalized for by the improved normalization process, the resulting particular gene NASR values are still greatly improved, relative to prior art produced particular gene NASR values, by virtue of being more validly, completely, and accurately normalized. In addition, the more completely defined and normalized microarray and non-microarray assay systems, provides a greatly improved base from which to further improve and define the microarray and non-microarray gene expression analysis and gene expression comparison assays.

For a particular microarray or non-microarray assay, in the event that a pertinent NF assay value cannot be determined and is therefore not known, a reasonably estimated value for that unknown NF can be used in the normalization process in combination with other determined CNFs and UNFs, to produce NASR values which have utility, and which are improved relative to prior art produced particular gene NASR values. Each such reasonably estimated NF should be identified, and the basis for the reasonable estimated NF value should be described.

Normalization of SAGE and other Clone Counting Method Measured Particular Gene Expression Assay Results for Differences in Cell Sample RNA Contents: Measuring and Normalizing for the Cell Sample Total mRNA number (STM) and STMR.

Here the discussion will emphasize the SAGE clone counting method, but the discussion also applies directly to other clone counting methods. A SAGE particular gene expression analysis results is expressed in terms of the particular gene mRNA frequency (mF) in the cell sample clone library of interest, and a SAGE gene expression comparison result for a particular gene is expressed in terms of the particular gene comparison mF ratio or mFR. A particular gene mFR value is equal to, (the particular gene mF value for one cell sample÷the particular gene mF value for the other compared cell sample). Prior art believes and practices that a SAGE measured particular gene mFR value is equal to the T-DGER value for the gene in the compared cell samples. However this prior art belief and practice is true only when the STMR value for the SAGE assay is equal to one or nearly one. When the SAGE assay STMR≠1, then the particular gene mFR value is not equal to the T-DGER for the gene, and the mFR value deviates from biological accuracy to the same extent that the STMR value deviates from one. Therefore, when the SAGE assay STMR value≠1, then the SAGE measured particular gene mFR or DGER value must be normalized for the STMR≠1 value in order to obtain a biologically correct DGER value. As discussed earlier, the amount of mRNA per cell is often significantly different for compared cell samples, and therefore STMR values which differ significantly from one are common for SAGE assays. Prior art SAGE and other clone counting practice does not determine or normalize for the STM or STMR. The direct determination of SAGE assay STM and STMR values, and the normalization of SAGE assay results for the STM and STMR values is discussed below.

The STM value for a cell sample is equal to the total number of mRNA molecules of all kinds which are present in a cell, or the average total number of mRNA molecules of all kinds per cell which are present in a cell sample. Prior art often measures the amount of mRNA present in eukaryotic cells and cell samples in terms of the fraction of the cell or cell sample total RNA which consists of mRNA molecules which possess a significant poly A tail, i.e. PA mRNA molecules. This fraction varies for different eukaryotic and mammalian cell types, and is reported to range from 1% to 5%. It is generally believed that the vast majority of the mRNA molecules present in each mammalian cell consists of PA mRNA. For simplicity herein, the fraction of total RNA which consists of mRNA is termed the % mRNA fraction. Very few solid % mRNA values are available for different eukaryotic and mammalian cell types. Prokaryotic cell mRNAs rarely possess significant PA tracts, and are reported to have % mRNA values of 1% to 4% or so.

Prior art believes that virtually all of the mRNA which is present in a eukaryotic cell or cell sample is associated with a significant PA tract, and that only a very small fraction of the cell mRNA does not possess a significant PA tract. In addition, prior art believes that the nucleotide length distribution of eukaryotic cell mRNA molecules is approximately normal and that for mammalian cells the average mRNA nucleotide length is about 1800 nucleotides. Other non-mammalian eukaryotic cell average mRNA nucleotide lengths have been reported to be somewhat shorter. A quantitative measure of the STM value for a cell sample can be obtained when: a) the cell mRNA nucleotide length distribution is normal or nearly normal; b) the cell mRNA average nucleotide length is known; c) the cell sample's T-RNA per cell content or average T-RNA per cell content is known; d) the % mRNA value for the cell sample is known. As discussed extensively earlier, the T-RNA content of different cells and cell samples is known to vary greatly for the same and different cell types, and is known to differ significantly for the same cells in different stages of the cell cycle. An example of the determination of the STM value for a cell sample is discussed below. The example involves a hypothetical mammalian cell sample which has a % PA mRNA value of 2% and a T-RNA per cell content of 15 picograms (Pg) per cell.

For this example the STM value is determined as follows. (i) Isolate undegraded T-RNA from the cell sample. (ii) Quantitate the amount of T-RNA recovered and isolate the PA mRNA fraction by standard prior art oligo dT or poly U based affinity chromatography and ensure that the amount of cell ribosomal RNA associated with the isolated PA mRNA prep is not significant. (iii) Quantitate the amount of PA mRNA recovered from the known amount of cell T-RNA, and determine the fraction of the T-RNA which consists of PA mRNA molecules. Here, (the % mRNA value)=(the amount of PA mRNA isolated÷the amount of T-RNA processed)×100. Here it will be assumed that the % mRNA value for the mammalian cell sample is 2%. (iv) Therefore 2% of the T-RNA of each cell consists of PA mRNA, and since each cell contains 15 Pg of T-RNA then each cell contains (0.02×15 Pg) or 0.3 Pg of mRNA. (v) An average mRNA is 1800 nucleotides long and has a mass of nearly 10⁻⁶ Pg, and therefore the cell sample STM value is equal to (0.3 Pg of mRNA per cell)÷(10⁻⁶ Pg mRNA per molecule) or 3×10⁵ mRNA molecules per cell or 3×10⁵ average mRNA molecules per cell. (vi) For a cell sample comparison, (the STMR value)=(the STM value for one cell sample)÷(the STM value for another compared cell sample).

An alternative method for determining the STM value for a cell sample also relies on the presence of a significant PA tract on eukaryotic mRNA molecules. This method is illustrated below and assumes that the T-RNA per mammalian cell value is 15 Pg. For this example the cell STM value is determined as follows. (a) Isolate T-RNA from the cells. This T-RNA may be significantly degraded. (b) Produce a properly designed labeled poly dT or poly U probe with a known signal specific activity which is measured in terms of signal activity per probe molecule. (c) Hybridize a molar excess of the labeled probe to the cell mRNA which is present in a known amount of cell T-RNA, and then remove all or essentially all non-hybridized probe from the T-RNA. (d) Determine the number of labeled probe molecules which hybridized with the known amount of T-RNA. This equals (the total amount of hybridized probe label signal associated with the known amount of T-RNA)÷(the amount of label signal associated with one labeled probe molecule). (e) Determine the number of sample cells represented by the amount of T-RNA in the hybridization mixture. This is equal to (the mass in Pg of cell T-RNA present in the hybridization mixture)÷(15 Pg per cell). (f) Determine the cell STM value. Here the (STM value)=(the total number of labeled probe molecules which hybridized to the known amount of cell T-RNA)÷(the total number of sample cells represented by the known amount of T-RNA present in the hybridization mixture). (g) One of skill in the art will recognize that the nucleotide length and composition of the labeled poly dT or Poly U molecules used is important for obtaining accurate STM values and will design probes accordingly. One of skill in the art will also recognize that the presence of one or more non-T or U nucleotides at the 3′ end of the poly dT or poly U labeled probe can facilitate this process.

As discussed later, a cell sample STM value can also be measured by using SAGE and other clone counting methods in conjunction with artificial housekeeping genes. Note that clone counting methods generally rely on the presence of significant PA tracts on mRNA molecules.

Determination of STM values for prokaryotes in general is probably possible but is very complex.

A SAGE or other clone counting method measured gene mF value can be normalized for the assay STM global NF to obtain the abundance value for the particular gene in the analyzed cell sample. This can be done using the relationship, (the normalized SAGE assay measured particular gene mF value)=(the particular gene abundance value in the analyzed cell sample library)=(the SAGE assay measured particular gene mF value)×(the assay STM value). This assumes that the SAGE assay measured particular gene mF value is properly normalized for all other pertinent NFs.

A SAGE measured cell sample comparison particular gene mFR value can be normalized for the assay STMR NF value to obtain the T-DGER value for the particular gene in the cell sample comparison. This can be done by using the relationship, (The normalized SAGE measured particular gene mFR value)=(the particular gene comparison T-DGER value)=(the SAGE assay measured particular gene comparison mFR value)×(the assay STMR value). This assumes that the SAGE assay measured particular gene comparison mFR value is properly normalized for all other pertinent NFs.

The Use of the Artificial Housekeeping Gene (AHG) Approach for Simplifying and Improving the Determination of, and Normalization for, Pertinent UNFs, and CNFs for SAGE and Other Clone Counting Methods.

The UNFs STM and STMR, and the CNFs associated with sampling statistics and sequencing error, are pertinent for SAGE and other clone counting method assays. For simplicity the SAGE and other clone counting methods will be referred to as the SAGE method analysis or assay unless otherwise noted. Prior art does not determine the assay STM or STMR assay values for each cell sample analyzed. Prior art SAGE practice for determining a particular gene mFR value assumes that the STM values associated with each compared cell sample are the same (26, 27). As discussed, extensively earlier, the STM values of SAGE compared cell samples often vary significantly. Prior art SAGE methods for determining the statistically significant number of clones from a cell sample library to analyze for a SAGE analysis, are based on prior art estimates of cell sample STM values which are assumed to be the same for each cell sample analyzed. Further, prior art SAGE methods for determining the assay variability associated with each assay measured particular gene mRNA mF or mFR value, are also based on these estimated STM values which are assumed to be essentially the same for each SAGE analyzed cell sample. Again, it is known that the STM values for prior art SAGE analyzed cell samples often differ significantly. In addition, the estimated STM value used by the prior art is supposed to represent the STM value associated with a typical sample cell type. The actual STM for particular cell sample types is not determined or known, and it is likely that such estimated STM values differ significantly from the true SAGE assay STM value. As a result of these factors, the assay error for each SAGE measured particular gene mF or mFR value which is associated with the sampling statistics cannot be known for such prior art results. In addition, such error varies with the abundance level of the particular gene mRNA in each cell sample. Prior art methods do not provide a means to directly determine the assay values for such error which is associated with different particular gene mRNA abundance levels. The earlier described Artificial Housekeeping Gene (AHG) approach can be used to simplify and improve the determination of and the normalization for the SAGE and other clone counting methods assay values for STM, STMR, and sampling statistics error.

The direct determination of the STM value for a cell sample and the STMR value for a cell sample comparison was earlier described. Also described was the determination of a cell sample RCN value for a cell sample RNA, and the RCNR value for a cell sample comparison. For a SAGE or other clone counting method assay, the SAGE RCN value is equal to the number of intact cell CEs which are associated with the aliquot of cell sample T-RNA or isolated mRNA which is used to produce the cell sample clone library. It is significantly simpler to determine the cell sample RCN value, than it is to directly determine the cell sample STM value. Once the RCN value for a cell sample is known it is possible to use the exogenous standard AHG approach in order to design a SAGE cell sample comparison analysis so that it is not necessary to directly determine the STM or STMR values for the compared cell samples. Such an assay can take a variety of forms. Following is a preferred form of such an assay. (a) Determine the intact cell CE value for each compared cell sample T-RNA. (b) Isolate T-RNA from each compared cell sample. (c) Determine the RCN value for each compared cell sample T-RNA aliquot which is used to produce the compared cell sample clone libraries. The T-RNA is preferred here because it is much simpler to isolate cell sample T-RNA than it is cell sample mRNA, and the intact cell CE value for T-RNA is much easier to determine than the intact cell CE value for mRNA. (d) To each cell sample T-RNA aliquot whose RCN is known, add a known mole amount of each of one or more different exogenous standard mRNAs. Here these exogenous standard mRNAs are termed AHG mRNAs. As discussed earlier, the ratio of the mole amounts of one AHG type added to compared cell sample RNA aliquots, is termed the sample AHG mRNA mole ratio, or SMR. For the different AHG mRNAs present the SMR value may vary, but must be known. Preferably add different known mole amounts of each different AHG mRNA type, and such added mole amounts should range from an abundance of one AHG mRNA molecule per cell sample T-RNA CE or less, to around 1000 AHG mRNA molecules per cell sample T-RNA CE or more. The known abundance value for one AHG mRNA type in one cell sample T-RNA, may be the same or different from the known abundance value for the AHG mRNA type in the other cell sample T-RNA. For simplicity, it will here be assumed that for one AHG mRNA type, the AHG mRNA abundance level for each compared cell sample is the same, or in other words that the AHG mRNA T-DGER value equals one for each different AHG mRNA. Different AHG mRNA types may have the same or different base composition, nucleotide length, or secondary structure. (e) Each cell sample T-RNA aliquot containing the known mole amounts of one or more different AHG mRNA molecules, is used to produce a cell sample cDNA prep. For each cell sample cDNA prep, the cell sample and AHG mRNAs present are simultaneously transcribed to produce cell sample mRNA cDNA transcripts and AHG mRNA cDNA transcripts. The R and Fmole assumptions are believed to be valid for both the cell sample mRNA and AHG mRNA cDNA transcripts present in each cell sample cDNA prep, for at least the SAGE pertinent portion of each cell sample mRNA and AHG mRNA which is present in the cell sample T-RNA aliquot mixture. (f) Each cell sample cDNA prep is then cloned to produce a cell sample cDNA clone library. It is believed that for each cell sample clone library that the R and Fmole assumptions are valid for at least the SAGE pertinent portion of each cell sample mRNA or AHG mRNA which was present in the cell sample T-RNA aliquot mixture. (g) The SAGE analysis is done on the AHG clone containing cell sample clone libraries. (h) For each cell sample particular gene and each AHG in the assay, determine the assay measured mF value. (i) For each cell sample, (the cell sample STM value)=(the known AHG abundance value divided by the measured AHG mF value) (j) Adjust each AHG and cell sample particular gene measured mF value for prior art CNFs which can affect the biological accuracy of the mF values. One such CNF is associated with the DNA sequencing error for the assay. (k) Using these adjusted particular gene and AHG mF values, determine the mFR value for each particular gene comparison and AHG comparison in the assay. Since the T-DGER value for each different AHG mRNA type in the assay is equal to one, the AHG mFR values for each different AHG type in the assay should be the same or nearly the same. (1) Here, the relationship between the known value for the AHG T-DGER and the assay measured value for the AHG mFR is described by (the AHG T-DGER)=(measured AHG mFR)(STMR). The STMR value is unknown for the SAGE cell sample comparison, and can be determined from the described relationship, (STMR)=(the known AHG T-DGER value)÷(measured AHG mFR value). Note that for the AHG comparison the UNF PAFR is not pertinent, and cannot influence the measured AHG mFR value, or the AHG determined STMR value for the assay. (m) For the assay, the STMR value associated with each measured AHG comparison mFR value, and particular gene comparison mFR in the assay, is the same. For each particular gene comparison in the assay the relationship between the unknown value for the particular gene comparison T-DGER, and the measured value for the prior art mFR, and the AHG determined value for the STMR, can be described as (particular gene comparison T-DGER value)=(measured particular gene mFR value)(AHG measured STMR value). This relationship and the AHG determined STMR value can then be used to normalize each particular gene comparison mFR value for the assay STMR value. Note that the UNF PAFR is pertinent for each cell sample particular gene comparison. Here it has been assumed that each particular gene mFR value is corrected for its associated PAFR value and other non-STMR associated assay variables which would cause the mFR value to deviate from biological accuracy. Note further that even when the particular gene comparison mFR value is not normalized for these non-STMR assay variable effects, the particular gene comparison mFR can be accurately and validly normalized for the AHG determined STMR value using the relationship, (the STMR normalized particular gene comparison measured unadjusted DGER value)=(particular gene comparison unadjusted mFR value)×(the AHG determined STMR value). (n) The incorporation into each SAGE analyzed cell sample clone library of multiple different AHGs with widely different but known AHG abundance values, can be used to determine the cell sample STM value and the STMR value for a cell sample comparison, and can also be used to determine the sampling statistics error associated with the measured AHG and particular gene mF values, and for measured AHG and particular gene mFR values. In other words, the sampling statistics error associated with the different cell sample abundance values, and with different combinations of compared abundance values, can be determined. Such error can be accurately determined when each cell sample STM value is known and when accurate values for the AHG or particular gene abundances are known. Such information is not known for prior art SAGE and other clone counting method assay results. Given such information, prior art statistical methods can be used to determine the statistical sampling error associated with the different AHG and particular gene mF and mFR values. (o) The above described discussion of the use of the AHG approach to determine the assay STM, STMR, and sampling statistics error, applies directly to the SAGE analysis of isolated cell sample mRNA instead of T-RNA. (p) For the above described approach, it is possible to determine the effect on the AHG determined STM and STMR values of different AHG nucleotide lengths, nucleotide sequence, nucleotide composition, and polynucleotide secondary structure, as well as other factors. If there are no such effects, then the measured adjusted AHG mFR values obtained for AHGs which have the same T-DGER value in the assay will be the same. Such effects would also affect the particular gene comparison mFR values in the assay, and these particular gene comparison mFR values can be normalized for such effects by determining normalization factors based on the AHG comparison mFR values quantitative response to such effects.

An essentially identical approach to the above described AHG approach adds known mole amounts of exogenous standard DNA molecules to a cell sample cDNA prep aliquot containing a known number of cell sample cDNA prep CEs, and then producing the cell sample clone library from the cell sample cDNA prep aliquot. The above discussion concerning the use of exogenous standard mRNAs for the AHG approach, applies directly to this approach of adding exogenous standard DNA molecules to the cell sample cDNA preps. This latter approach is less desirable because it is much more difficult to determine a cell sample cDNA prep CE value than a cell sample intact cell CE value for T-RNA or isolated mRNA.

Here, the AHG approach is discussed in terms of the use of added exogenous standard AHG mRNAs and DNAs. However, judiciously chosen endogenous cell sample mRNAs or DNAs which represent cell sample mRNAs which are not expressed in the cell sample, or a potential mRNA or other RNA sequence produced from the cell sample DNA, can also be used as AHG mRNAs or DNAs for this and other purposes.

Note that the above discussion applies to SGDS, DGDS, and DGSS, particular gene RNA transcript of any kind comparisons which can be measured by SAGE or some other clone counting method. Note further that the use of this AHG method for an MPSS assay is greatly complicated by the use of a PCR amplification step for the MPSS method.

Application of Discussions on NF Determination and Normalization and the Use of the AHG Approach to Microarray and Non-Microarray or Clone Counting SGDS, DGDS, and DGSS Gene Expression Analysis of Different RNA Types.

The discussions on the determination and normalization for, pertinent assay NF values, and the uses of the AHG approach for microarray and non-microarray and clone counting gene expression analysis methods are directly applicable to SGDS, DGDS, and DGSS comparisons of all kinds for viral, prokaryotic, eukaryotic, and standard RNA transcripts of all kinds. This includes all types of rRNA, tRNA, mRNA, siRNA, miRNA, snoRNA, antisense RNA, and other known and unknown RNAs. In addition these discussions also apply directly to the well known but rarely used hybridization based gene expression analysis methods such as the ELISA based and hydroxyapatite based methods.

B. Production of Improved Gene Expression Analysis and Gene Expression Comparison Analysis Results for Microarray, Non-Microarray, and Clone Counting Method SGDS, DGDS, and DGSS Comparisons of Viral, Prokaryotic, Eukaryotic, and Standard RNA Transcripts of all Kinds.

While the following discussion emphasizes and describes the production of invention improved results for the commonly used microarray and non-microarray methods, it will be apparent to one of skill in the art that the description can be directly and readily applied to producing invention improved results for the less commonly used methods, such as for example the ELISA, hydroxyapatite, and other gene expression analysis methods.

The practice of the invention produces assay results for microarray and non-microarray and clone counting method SGDS, DGDS, and DGSS, gene expression comparison analyzes of viral, prokaryotic, eukaryotic, and standard, RNA transcripts of all kinds, which are known to be improved relative to prior art produced assay results for microarray, non-microarray, and clone counting method SGDS, DGDS, and DGSS gene expression comparison analyzes of viral, prokaryotic, eukaryotic, and standard RNA transcripts. Such invention produced improved RNA transcript comparison results are produced by using a normalization process which is improved relative to prior art utilized normalization processes. The invention produces RNA transcript comparison assay results which are known to be improved in normalization, relative to prior art produced and normalized RNA transcript comparison assay results. Invention produced improved results are also known be improved in the known degree of valid normalization for pertinent assay variable associated CNFs and UNFs, relative to prior art produced and normalized assay results. As a result the invention produced RNA transcript comparison assay results are known to be improved in accuracy and/or quantitation and/or interpretability and/or reproducibility and/or intercomparability and/or utility, relative to prior art produced and normalized assay results.

The practice of the invention provides methods and means for producing such improved gene expression analysis comparison assay results for microarray, non-microarray, and clone counting method SGDS, DGDS, and DGSS assay comparisons of viral, prokaryotic, eukaryotic, and standard RNA transcripts of all kinds. Such RNA transcripts include all types of rRNA, tRNA, mRNA, siRNA, miRNA, snoRNA, antisense RNA, standard RNA, and other known and unknown RNA transcripts. Such improved RNA transcript comparison normalized assay results are produced by using normalization processes which are known to be improved, relative to prior art normalization processes. Such improved invention related normalization processes are improved by the identification of assay pertinent prior art unknown or unconsidered assay variable associated UNFs, and the determination of the assay values for the pertinent UNFs and the valid normalization of the assay results for the pertinent UNF assay values, and/or by the valid determination of assay values for pertinent CNFs and the valid normalization of the assay results for the pertinent CNF values, and/or by assay design considerations which facilitate and improve the normalization process.

Microarray, non-microarray, and clone counting method assays which utilize the methods and means of the invention to produce such improved assay results, are described below. These assays utilize assay design and measurement of the assay values for pertinent CNFs and UNFs in order to improve the assay normalization process. One or more of the identified UNFs is pertinent to and associated with a microarray, non-microarray, or clone counting method SGDS or DGDS or DGSS particular gene or standard RNA transcript of any kind comparison assay. One or more identified assay design solutions can be used to design a microarray, non-microarray, or clone counting method SGDS or DGDS or DGSS particular gene or standard RNA transcript of any kind assay comparison. The set of all microarray, non-microarray, and clone counting method SGDS, DGDS, and DGSS particular gene RNA transcript of any kind comparison assays, which can produce invention improved gene expression comparison results is very very large. The vast majority of prior art produced microarray, non-microarray, and clone counting method assays concern only the SGDS comparison of viral or prokaryotic or eukaryotic mRNA transcripts of all kinds. Even for these prior art assays which concern only the SGDS comparison of mRNA transcripts, the set of all microarray, non-microarray, and clone counting method assays which can produce improved gene expression comparison results by the practice of the invention, is very large.

Because the number of possible assay design permutations which can produce invention improved assay results is too large to practically describe, and because the prior art assays concern almost exclusively the SGDS comparison of mRNA transcripts, the following descriptions of improved assay design combinations which practice the invention, will focus primarily on microarray, non-microarray, and clone counting method SGDS particular gene mRNA transcript comparison assays. The following Tables 54-69, 75-90, 93-95, 97, and 99, reflect this focus. A large number of SGDS mRNA comparison assay design solution combinations which can produce invention improved assay results is presented in these Tables. This number represents only a small fraction of the possible design solution combinations which can produce such improved results. Herein, a design solution combination which produces an invention improved result is termed an improved design solution combination. Further, an invention improved assay result is termed an improved result.

Note that all or virtually all of the following described SGDS mRNA transcript comparison assay design solution combinations which produce improved assay results, also produce SGDS, DGDS, and DGSS RNA transcript of any kind comparison assay results which are improved. This will be discussed later.

C. Practice of the Invention for SGDS mRNA Transcript or mRNA Transcript cDNA or cRNA Equivalent Comparison Assays.

Improvement of the Prior Art Microarray Normalization Process for Direct label LPN Assays by Assay Design and Measurement of UNF and CNF Assay Values.

A large number of assay variables are associated with prior art microarray direct label LPN comparison assays. The great majority of such assays involve the comparison of cell sample fluorescent or radioactive type 1 LPN preps. For these microarray assays as many as thirteen different NFs may be pertinent for an assay. A small fraction of prior art microarray assays involve the comparison of cell sample fluorescent or radioactive type 2 LPN preps. For these type 2 assays, as many as eleven different NFs may be pertinent for an assay.

In order to accurately and completely normalize particular gene RASR values produced by such directly labeled type 1 or type 2 LPN microarray assays, it is necessary to determine or know, an accurate quantitative value for each NF which is pertinent for the particular gene RASR, and then to normalize the particular gene RASR for the pertinent NF values. The determination of such pertinent NF quantitative values was discussed earlier. While determination of assay values for global NFs is generally practical, such determination can still be complex, as for example, the determination of the assay SCR. Determination of the C-HKR global NF assay values is relatively simple. In contrast, the determination of the assay value for particular gene non-global NFs can be quite complex, and has been described earlier. The non-global CNFs can be determined and normalized for in a straightforward way, using standards in combination with well established prior art methods which are currently in routine use, if the necessary normalization assumptions are valid. Absent the validity of these required normalization assumptions this is not possible. The determination of the assay value for particular gene non-global UNFs is much more complex. Determination of particular gene assay values for the non-global UNF MLDR can be done by a combination of inference and measurement as described earlier. Determination of the PL-HKR and PS-HKR values is more complex and requires information which is not presently well known, but can be obtained. Absent such information, the PL-HKR and PS-HKR values cannot be directly measured for many assay situations. Determination of the PSAR is complex. In addition, it is impractical to determine the PAFR or PSSR assay values for each particular gene in an assay, even for low density microarrays. Determination of the LLSR can also be complex.

It is useful to describe the pertinent NFs which are associated with prior art microarray directly labeled cell sample LPN prep comparison assays. The vast majority of prior art microarray assays compare cell sample fluorescent or radioactive oligo dT or random primed type 1 LPNs. The large majority of these prior art assays compare oligo dT primed LPNs. A small fraction of all prior art microarray assays compare cell sample fluorescent or radioactive specific gene primed type 1 LPNs. The pertinent UNFs associated with each of these different primer microarray assays is presented in Table 47. Table 48 presents the pertinent CNFs which are associated with the prior art microarray use of these primers. Note that for two label assays the CNF C-HKR is not pertinent for any of these assays, and that the PAFR UNF is not pertinent for random or SG primed assays which compare cell sample T-RNAs. Tables 47 and 48 illustrate the pertinent UNFs and CNFs which are associated with virtually all prior art microarray assays. Note that the commonly considered prior art NF ARR is incorporated into the SCR UNF.

TABLE 47
UNFs Associated with Prior Art Microarray Assay Comparisons of
Fluorescent or Radioactive Type 1 LPNs

| | Pertinent UNFs | | Pertinent UNFs When | |
|---|---|---|---|---|
| | When Comparing Isolated | | Comparing Cell | |
| | Cell Sample mRNAs | | Sample T-RNAs | |
| | *One Label | *Two Label | One Label | Two Label |
| Primer Used | Assay | Assay | Assay | Assay |
| Oligo dT | SCR | SCR | SCR | SCR |
| | PAFR | PAFR | PAFR | PAFR |
| | MLDR | MLDR | MLDR | MLDR |
| | PL-HKR | PL-HKR | PL-HKR | PL-HKR |
| | PS-HKR | PS-HKR | PS-HKR | PS-HKR |
| | PSAR | PSAR | PSAR | PSAR |
| | PSSR | PSSR | PSSR | PSSR |
| Random | SCR | SCR | SCR | SCR |
| Or | PAFR | PAFR | — | — |
| SG Primer | MLDR | MLDR | MLDR | MLDR |
| Mixture | PL-HKR | PL-HKR | PL-HKR | PL-HKR |
| | PS-HKR | PS-HKR | PS-HKR | PS-HKR |
| | PSAR | PSAR | PSAR | PSAR |
| | PSSR | PSSR | PSSR | PSSR |

*A two label assay refers to a microarray assay where the compared LPNs are associated with different labels, and the compared LPNs are mixed together for the assay. A one label assay refers to a microarray assay where only one of the compared LPN preps is present in a microarray hybridization solution, and each assay is associated with two separate hybridization solutions.

TABLE 48
CNFs Associated with Prior Art Microarray Assay Comparisons of
Fluorescent or Radioactive Type 1 LPNs

| | Pertinent CNFs | | Pertinent | |
|---|---|---|---|---|
| | When Comparing | | CNFs When | |
| | Isolated Cell | | Comparing Cell | |
| | Sample mRNAs | | Sample T-RNAs | |
| | One Label | Two Label | One Label | Two Label |
| Primer Used | Assay | Assay | Assay | Assay |
| Oligo dT | C-HKR | Spatial | C-HKR | Spatial |
| or | Spatial | Print Tip | Spatial | Print Tip |
| Random | Print Tip | Print Plate | Print Tip | Print Plate |
| or | Print Plate | Intensity | Print Plate | Intensity |
| SG Primer | Intensity | Scale | Intensity | Scale |
| Mixture | Scale | | Scale | |

A very small fraction of all prior art microarray assays compare cell sample fluorescent or radioactive oligo dT or SG primed type 2 LPNs. The pertinent UNFs and CNFs associated with these different primer type 2 LPN comparisons are presented in Tables 49 and 50. Note that for these type 2 assays the non-global UNFs MLDR, PSAR, and PSSR are not pertinent for these assays. Further, the global CNF C-HKR is not pertinent for either two label assay, and the non-global UNF PAFR is not pertinent for either of the SG primed T-RNA assays.

TABLE 49
UNFs Associated with Prior Art Microarray Assay Comparisons of
Fluorescent or Radioactive Type 2 LPNs

| | Pertinent UNFs | | Pertinent | |
|---|---|---|---|---|
| | When Comparing | | UNFs When | |
| | Isolated Cell | | Comparing Cell | |
| | Sample mRNAs | | Sample T-RNAs | |
| | One Label | *Two Label | One Label | Two Label |
| Primer Used | Assay | Assay | Assay | Assay |
| Oligo dT | SCR | SCR | SCR | SCR |
| | PAFR | PAFR | PAFR | PAFR |
| | PL-HKR | PL-HKR | PL-HKR | PL-HKR |
| | PS-HKR | PS-HKR | PS-HKR | PS-HKR |
| | LLSR | LLSR | LLSR | LLSR |
| SG Primer | SCR | SCR | SCR | SCR |
| Mixture | PAFR | PAFR | — | — |
| | PL-HKR | PL-HKR | PL-HKR | PL-HKR |
| | PS-HKR | PS-HKR | PS-HKR | PS-HKR |
| | LLSR | LLSR | LLSR | LLSR |

*For a two label assay, the compared differently labeled LPNs are mixed together into one hybridization solution for the assay.

TABLE 50
CNFs Associated with Prior Art Microarray Assay Comparisons of
Fluorescent or Radioactive Type 2 LPNs

| | Pertinent CNFs | | Pertinent | |
|---|---|---|---|---|
| | When Comparing | | CNFs When | |
| | Isolated Cell | | Comparing Cell | |
| | Sample mRNAs | | Sample T-RNAs | |
| | One Label | *Two Label | One Label | Two Label |
| Primer Used | Assay | Assay | Assay | Assay |
| Oligo dT | C-HKR | — | C-HKR | — |
| or | Spatial | Spatial | Spatial | Spatial |
| SG Primer | Print Tip | Print Tip | Print Tip | Print Tip |
| Mixture | Print Plate | Print Plate | Print Plate | Print Plate |
| | Intensity | Intensity | Intensity | Intensity |
| | Scale | Scale | Scale | Scale |

Here, the non-global PSAR is replaced by the global UNF LLSR. This is a very positive exchange. The LLSR is a global assay variable which has the same assay value for all different particular gene comparisons in the assay, unlike the PSAR, which can have many different particular gene PSAR values associated with the assay. In addition the LLSR assay value can often be readily and directly determined experimentally, whereas the process of determining the PSAR is often indirect, and involves a process of inference and experimental measurement. As a consequence the LLSR value should be more accurate and precise than the PSAR values.

Each of the prior art microarray assay situations described in Tables 47 through 50 represents a prior art microarray practice general assay situation, and the UNFs and CNFs which must be determined and normalized for, in order to obtain improved microarray measured particular gene NASR and N-DGER values, and biologically accurate particular gene NASR values. In order to obtain such improved particular gene NASR values for prior art practice microarray assays, the following improvements in the prior art practice normalization process are required. (i) it is necessary to use an improved normalization approach which can be known to be valid, or to know that the key prior art normalization assumptions are valid, in order to determine the pertinent CNF values and normalize the particular gene RASR values for them. (ii) it is necessary to use an improved overall process for the more complete and accurate normalization of microarray assay measured particular gene RASR values, which includes the identification of the pertinent UNFs and CNFs for the assay, the valid and accurate determination of pertinent UNF and CNF assay values, as well as the valid and accurate normalization for the pertinent UNF and CNF values.

Prior art microarray practice does not determine the assay value for, or normalize particular gene RASR values for, global or non-global UNFs. The great majority of prior art microarray gene expression comparison assays utilize moderate to high density microarrays, and involve the comparison of cell sample fluorescent or radioactive type 1 LPN preps produced by oligo dT or random priming. The great majority of these microarray assays compare oligo dT primed LPN preps. For such assays, as many as thirteen NFs may be pertinent to the assay, and seven of these are UNFs. Each UNF can cause an assay measured particular gene RASR value to deviate significantly from biological accuracy when the UNF value deviates significantly from one. Table 51 presents the previously discussed estimates of the magnitudes of the deviations from one which are believed to commonly occur for the UNFs and CNFs of prior art microarray assays, as well as the commonly claimed measurement accuracies for prior art microarray assays. It is likely that most prior art microarray assays are associated with at lease one UNF which does not equal one, and many, if not most, are likely to be associated with more than one UNF≠1 value. In the context of the measurement accuracy of a typical prior art microarray assay, the deviation of even one of these UNFs from one is large enough to significantly affect the quantitative value and interpretation of a prior art measured particular gene N-DGER value.

TABLE 51
Estimated Magnitude of Deviation of NFs from One for a Prior Art Microarray
Assay Comparing Fluorescent or Radioactive Type 1 Direct Label LPNs

| | | ^{a}Estimated | | |
|---|---|---|---|---|
| | | Deviation of NF Value From | | |
| | | One For A Typical Prior | | |
| | | Art Microarray Assay | | Measurement |
| | | Commonly Occurring | Plausible | Accuracy of Prior |
| Type of NF | | Conservative Estimated | Potential | Art Microarray |
| UNF | CNF | Deviation | Deviation | Assays |
| SCR | | 6 Fold | 20-25 Fold | The measurement of |
| PAFR | | 1.33 Fold | 3 Fold | accurate N-DGER |
| MLDR | | 3 Fold | 10-20 Fold | values to within ±1.2 |
| PL-HKR | | 1.5 Fold | 3 Fold | to 4 fold is often |
| PS-HKR | | 2 Fold | >5 Fold | claimed. Generally, |
| PSAR | | 2 Fold | >5 Fold | the claim is ±1.5 to 2 |
| PSSR | | 1.5 Fold | >5 Fold | fold. |
| LLSR^{b} | | 1.5 Fold | >5 Fold | |
| | C-HKR | 2 Fold | 3-5 Fold | |
| | Spatial | 2 Fold | 3-5 Fold | |
| | Print Tip | 2 Fold | 3-5 Fold | |
| | Print Plate | 2 Fold | 3-5 Fold | |
| | Intensity | 2 Fold | 3-5 Fold | |
| | Scale | 2 Fold | 3-5 Fold | |

^{a}An NF deviation of 2 fold from one will cause a 2 fold deviation from biological accuracy.
^{b}LLSR applies only to Type 2 LPN comparisons.

Therefore such deviations from one have significant practical importance for the interpretation of prior art produced N-DGER values and the future production of biologically accurate microarray measured N-DGER values. Further, prior art microarray practice does not determine the assay values for the UNFs, and as a result it cannot be known whether a prior art measured particular gene RASR value requires normalization for UNFs or not. Therefore it is necessary to first identify the UNFs which are pertinent for an assay, and then to determine a quantitative measure of each pertinent UNFs assay value in order to determine whether UNF normalization is necessary, and then to normalize the particular gene RASR value for the UNF values, if UNF normalization is required. For a typical microarray assay the requirement to determine and normalize for the assay pertinent UNF values adds a very significant amount of complexity and effort to the microarray assay, relative to the prior art microarray assay. In addition, a significant amount of systematic measurement error and noise may be associated with the experimentally determined UNF values, and their use for normalization. Further, as discussed above, it is not practical to determine the assay UNF PAFR or PSSR values for more than a few gene comparisons in an assay, and it is often not feasible to determine the PL-HKR and PS-HKR assay values. The use of the improved method for determining and normalizing for the CNFs spatial, print tip, print plate, intensity, and scale, also adds additional complexity and effort to the microarray assay, relative to prior art microarray practice. These consideration make it very desirable, if not necessary, to simplify the determination of pertinent CNF and UNF values and the normalization process as much as possible, and to eliminate the necessity for experimentally determining as many UNFs and CNFs as possible. Here it is particularly desirable to eliminate the need to determine the assay values for those UNFs or CNFs which cannot be determined, such as PAFR and PSSR, and those which are difficult to determine.

Earlier sections extensively discussed the underlying basis for each microarray assay UNF, and the assay situations under which each UNF is pertinent. As a result of this it is possible to identify the assay factors which can and must be controlled for different assay situations, in order to simplify the process of determining the pertinent UNF values, and normalizing for them. This knowledge makes it possible to knowingly design microarray assays which do not require the direct determination of certain UNFs and CNFs, including PAFR, MLDR, PL-HKR, PS-HKR and PSSR, in order to validly normalize for these NFs. The overall result of such designs is a simplified version of the improved microarray normalization process. This can be accomplished by judicious assay design and measurement, as is discussed below.

The various design approaches which will result in an improved normalization process relative to prior art normalization processes, are presented in Table 52. The successful implementation of any one of the Table 52 design approaches 1-8 will produce a normalization process which can be known to be improved, relative to prior art normalization practices. The successful implementation of Table 52 design approach 9 will produce microarray assay results which are known to contain fewer NF related false negative results than prior art microarray results.

TABLE 52
Design Approaches for Improving the Gene Expression Assay Normalization
Process Relative to the Prior Art Normalization Process

| | |
|---|---|
| (1) | Design the assay to validly normalize for pertinent CNFs. |
| (2) | Design the assay to normalize for one or more pertinent UNFs. |
| (3) | Design the assay to validly normalize for pertinent CNFs, and to normalize for one or |
| | more pertinent UNFs. |
| (4) | Design the assay so certain NFs are known to be not pertinent to the assay and can be |
| | ignored during the normalization process. |
| (5) | Design the assay so certain pertinent NFs are known to have an assay value of one |
| | and can be ignored during the normalization process. |
| (6) | Design the assay to maximize the number of pertinent NFs which can be ignored |
| | during the normalization process. |
| (7) | Design the assay so that the assay values for the pertinent NFs are as easy as possible |
| | to determine. |
| (8) | Design the assay so that the normalization process is as easy and straightforward as |
| | possible. |
| (9) | Design the assay to minimize or eliminate the occurrence of UNF and CNF related |
| | false negative results and their associated RDMs. |

Prior art microarray assay design is not standardized, and there are a wide variety of different microarray designs practiced by the prior art. The improvement of the normalization process for each of these prior art practice assay designs will be discussed. The design solutions or design components which can be used to produce improved microarray assay normalization are presented in Table 53. Each of these design solutions or design components reflects an aspect of microarray assay design which either directly or indirectly impacts on an assay pertinent NF and/or the simplification of the normalization process. Different combinations of these design solutions or design components can be used to describe an overall microarray assay. Certain of these design solutions are discussed and further defined below.

Design Solutions 1, 2, 3.

Prior art cDNA microarrays were discussed earlier. Such arrays generally contain only one CDP sequence for each different gene mRNA of interest, and the CDP nucleotide length or complexity ranges from roughly 200 to well over 1000 nucleotides. Many oligonucleotide microarrays are design solution 2 arrays, and the nucleotide lengths and complexities of the oligonucleotide CDPs range from about 30 to about 70 nucleotides. As an example, GE codelink arrays contain oligonucleotide CDPs about 30 nucleotides long, while the Agilent arrays are about 60 nucleotides long. Design solution 3 arrays are represented by Affymetrix arrays. These arrays contain oligonucleotide CDPs which are about 25 nucleotides long, and also contains as many as 10 or more different CDPs specific for each particular gene mRNA of interest.

TABLE 53
Design Solutions for Improving the Prior Art Microarray Assay Normalization
Process and the Assay Measured Particular Gene NASR Values: Directly Labeled LPNs

| | | NFs Which | | | |
|---|---|---|---|---|---|
| | | Can Be | | Reason For | |
| | | Ignored During | | Ignoring NFs | |
| | | Normalization | | (NP = Not Pertinent) | |
| Assay Design Solutions | | UNF | CNF | UNF | CNF |
| (1) | Use cDNA microarray. | — | — | — | — |
| (2) | Use an oligonucleotide microarray which | — | — | — | — |
| | contains only one CDP sequence specific | | | | |
| | for each different gene mRNA to be | | | | |
| | detected. | | | | |
| (3) | Use an oligonucleotide microarray which | — | — | — | — |
| | contains multiple CDP sequences | | | | |
| | specific for each different gene mRNA to | | | | |
| | be detected. | | | | |
| (4) | Use (a) Radioactive Label | — | — | — | — |
| | (b) Non-radioactive label | | | | |
| (5) | Compare (a) Type 1 LPNs | LLSR | — | NP | — |
| | (b) Type 2 LPNs | MLDR | | NP | |
| | | PSAR | | NP | |
| (6) | Use standards to validly normalize for | — | — | — | — |
| | pertinent global and non-global CNFs. | | | | |
| (7) | Use prior art method to normalize for | — | — | — | — |
| | pertinent global and non-global CNFs, | | | | |
| | after establishing the validity of the prior | | | | |
| | art normalization method for the assay. | | | | |
| (8) | Use AHG and/or other standards to | — | — | — | — |
| | determine and normalize for | | | | |
| | (a) SCR | | | | |
| | (b) LLSR | | | | |
| | (c) PSAR | | | | |
| (9) | Compare oligo dT primed LPNs | — | — | — | — |
| | produced from | — | — | — | — |
| | (a) Cell sample T-RNAs | | | | |
| | (b) Cell sample isolated mRNAs | | | | |
| (10) | Compare Specific Gene (SG) primed | PAFR | — | NP | — |
| | LPNs produced from | — | — | — | — |
| | (a) Cell sample T-RNAs | | | | |
| | (b) Cell sample isolated mRNAs | | | | |
| (11) | Compare random primed LPNs made | PAFR | — | NP | — |
| | from cell sample T-RNAs. | | | | |
| (12) | Compare random primed LPNs made | — | — | — | — |
| | from cell sample isolated mRNAs. | | | | |
| (13) | Use (a) One label for assay | — | — | — | — |
| | (b) Two labels for assay | — | C-HKR | — | C-HKR = 1 |
| (14) | Use low enough LPN Label Density (LD) | PSSR | — | NP | — |
| | so that LD effects are essentially absent. | — | — | — | — |
| (15) | Synthesized LPN nucleotide lengths for | — | — | — | — |
| | the LPN molecules in a cell sample LPN | — | — | — | — |
| | prep are | | | | |
| | (a) The same | | | | |
| | (b) Different | | | | |
| (16) | The average synthesized LPN nucleotide | MLDR* | — | MLDR = 1 | — |
| | lengths of compared cell sample LPN | PL-HKR* | | PL-HKR = 1 | |
| | preps are | PS-HKR* | | PS-HKR = 1 | |
| | (a) The same | — | — | — | — |
| | (b) Different | | | | |
| (17) | Compared cell sample LPN preps are | MLDR* | — | MLDR = 1 | — |
| | synthesized and then adjusted to have | PL-HKR* | | PL-HKR = 1 | |
| | nucleotide lengths which are somewhat | PS-HKR* | | PS-HKR = 1 | |
| | longer than the longest CDP on the | — | — | — | — |
| | microarray, and which | | | | |
| | (a) Have the same average LPN | | | | |
| | nucleotide lengths | | | | |
| | (b) As (a) except that the average | | | | |
| | nucleotide lengths are much smaller than | | | | |
| | in (a) | | | | |
| (18) | Synthesized LPN nucleotide lengths for | MLDR* | — | MLDR = 1 | — |
| | the compared particular gene LPNs are | PL-HKR* | | PL-HKR = 1 | |
| | (a) The same | PS-HKR* | | PS-HKR = 1 | |
| | (b) Different | — | — | — | — |
| (19) | Synthesized LPN nucleotide lengths and | MLDR | — | MLDR = 1 | — |
| | nucleotide sequences are the same or | PL-HKR | | PL-HKR = 1 | |
| | essentially the same for all compared | PS-HKR | | PS-HKR = 1 | |
| | particular gene LPNs in the assay. | | | | |
| (20) | Synthesized LPN nucleotide lengths and | MLDR | — | MLDR = 1 | — |
| | nucleotide sequences are the same or | PL-HKR | | PL-HKR = 1 | |
| | essentially the same for less than all | PS-HKR | | PS-HKR = 1 | |
| | compared particular gene LPNs in the | | | | |
| | assay. | | | | |
| (21) | Compare synthesized particular gene | MLDR | — | MLDR = 1 | — |
| | LPNs which are equal in nucleotide length | PL-HKR | | PL-HKR = 1 | |
| | to each particular gene's undegraded | PS-HKR | | PS-HKR = 1 | |
| | mRNA nucleotide length. | | | | |
| (22) | Compare directly in the microarray assay | PAFR | — | NP | — |
| | hybridization solution labeled mRNA | | | | |
| | LPNs produced from | | | | |
| | (a) Cell sample T-RNA | | | | |
| | (b) Cell sample isolated mRNA | | | | |
| (23) | Labeled mRNA LPN nucleotide lengths in | — | — | — | — |
| | a cell sample mRNA LPN prep are | — | — | — | — |
| | (a) The same | | | | |
| | (b) Different | | | | |
| (24) | The average nucleotide lengths of | MLDR* | | MLDR = 1 | |
| | compared cell sample mRNA LPN preps | PL-HKR* | | PL-HKR = 1 | |
| | are | PS-HKR* | | PS-HKR = 1 | |
| | (a) The same | — | — | — | — |
| | (b) Different | | | | |
| (25) | Compared cell sample mRNA L-LPN | MLDR* | — | MLDR = 1 | — |
| | preps are adjusted to have nucleotide | PL-HKR* | | PL-HKR = 1 | |
| | lengths which are somewhat longer than | PS-HKR* | | PS-HKR = 1 | |
| | the longest CDP on the microarray, and | — | — | — | |
| | which have | | | | |
| | (a) The same or nearly the same average | | | | |
| | nucleotide lengths | | | | |
| | (b) Much smaller average nucleotide | | | | |
| | lengths than in (a), which are the same | | | | |
| (26) | mRNA LPN nucleotide lengths for | MLDR* | — | MLDR = 1 | — |
| | compared particular gene mRNA LPNs | PL-HKR* | | PL-HKR = 1 | |
| | are | PS-HKR* | | PS-HKR = 1 | |
| | (a) The same | — | — | | — |
| | (b) Different | | | | |
| (27) | mRNA LPN nucleotide lengths and | MLDR | — | MLDR = 1 | — |
| | nucleotide sequences are the same for all | PL-HKR | | PL-HKR = 1 | |
| | compared particular gene mRNA LPNs in | PS-HKR | | PS-HKR = 1 | |
| | assay. | | | | |
| (28) | mRNA LPN nucleotide lengths and | MLDR | — | MLDR = 1 | — |
| | nucleotide sequences are the same for less | PL-HKR | | PL-HKR = 1 | |
| | than all compared particular gene mRNA | PS-HKR | | PS-HKR = 1 | |
| | LPNs in assay. | | | | |
| (29) | Compare particular gene undegraded | MLDR | — | MLDR = 1 | — |
| | labeled mRNA LPNs. | PL-HKR | | PL-HKR = 1 | |
| | | PS-HKR | | PS-HKR = 1 | |
| (30) | For all particular gene comparisons of | PSAR | — | PSAR = 1 | — |
| | labeled mRNA LPNs, or cDNA LPNs, or | SCR | — | SCR = 1 | — |
| | cRNA LPNs, the assay value for the UNF | LLSR | — | LLSR = 1 | — |
| | (a) PSAR | | | | |
| | (b) SCR | | | | |
| | (c) LLSR | | | | |
| | is known to equal one. | | | | |
| (31) | Determine for each particular gene | — | — | — | — |
| | comparison the assay value for | | | | |
| | (a) MLDR | | | | |
| | (b) PL-HKR | | | | |
| | (c) PS-HKR | | | | |
| | (d) PSAR | | | | |
| | (e) LLSR | | | | |
| (32) | Each of the oligo dT or SG primed cDNA, | MLDR | — | MLDR = 1 | — |
| | or standardly produced cRNA, or labeled | PL-HKR | | PL-HKR = 1 | |
| | mRNA, compared cell sample LPN preps, | PS-HKR | | PS-HKR = 1 | |
| | has an average nucleotide length which is | | | | |
| | greater than the nucleotide length of | | | | |
| | undegraded mRNA molecules for one or | | | | |
| | more, but not all, different particular genes | | | | |
| | in the assay. | | | | |
| (33) | Use a cDNA microarray which contains | — | — | — | — |
| | only one CDP sequence for each different | | | | |
| | gene mRNA to be detected, and each such | | | | |
| | particular gene CDP sequence has a | | | | |
| | nucleotide length and nucleotide complexity | | | | |
| | which is equal to or preferably, significantly | | | | |
| | shorter than, the nucleotide length or | | | | |
| | complexity of the shortest gene undegraded | | | | |
| | mRNA in the assay. | | | | |
| (34) | Maximize the number of different pertinent | All that | All | NFs = 1 | NFs = 1 |
| | UNFs and CNFs which have an assay value | equal one | that | | |
| | equal to one or nearly one. | | equal | | |
| | | | one | | |

*Can ignore these UNFs when compared LPNs are produced from cell sample T-RNA, but may not be able to ignore these UNFs when the compared LPNs are produced from cell sample isolated mRNAs.

Design Solutions 4, 13.

As discussed earlier this includes only directly labeled LPNs.

Design Solutions 6, 8.

Endogenous particular gene mRNAs and exogenous standard mRNAs and labeled and unlabeled DNAs or cRNAs can be used for determining and normalizing for both CNFs and UNFs which are pertinent for an assay. This includes the determination of and normalization for the assay SCR value by the artificial housekeeping gene (AHG) approach.

Design Solution 7.

To accomplish this it must be determined for the particular assay that the prior art key normalization assumptions are valid.

Design Solutions 9, 10, 11, 12.

The optimal set of gene mRNA CDPs for the assay may be different for each different primer, and the type of primer should be taken into consideration when designing the gene mRNA CDP set for the assay. As an example, the comparison of oligo dT primed, or 3′ end targeted SG primed LPNs, necessitates the use of gene mRNA CDPs which can detect the LPN molecules which represent the 3′ end portion of each different gene mRNA. In contrast, the comparison of random primed LPNs from undegraded mRNA preps allows much more freedom in CDP design.

Design Solution 14.

This can be readily accomplished by controlling the amount of label which is associated with the LPN. In order to accomplish this for all compared particular gene LPNs, it must be kept in mind that in the same cell sample type 1 LPN prep, different particular gene LPNs can have significantly different label densities.

Design Solution 15.

In an LPN prep, a population of particular gene LPN molecules is associated with each particular gene which is represented in the LPN prep. Here, the same nucleotide lengths indicates that each different particular gene LPN molecule population in the LPN prep has the same average LPN molecule population nucleotide length when synthesized. Such LPNs can be produced using the earlier discussed controlled synthesis termination method. In contrast, different nucleotide lengths indicates that in the LPN prep, different particular gene LPN molecule populations have different average nucleotide lengths.

Design Solution 16.

The overall synthesized LPN molecule population average nucleotide length for an LPN prep reflects a complex average of all of the different particular gene LPN molecule populations which are present in an LPN prep.

Design Solution 17.

Compared cell sample synthesized LPN preps often are different in average nucleotide length. Prior art often adjusts the average nucleotide lengths of synthesized compared LPN preps to much smaller average nucleotide lengths. Prior art puts no emphasis on making these compared adjusted LPN preps have the same much smaller average nucleotide lengths, and rarely determines the average nucleotide lengths of the adjusted LPNs.

Design Solution 18, 19.

Note that it is possible for compared particular gene synthesized LPN molecule populations to have the same average nucleotide lengths, but not the same nucleotide sequences. This occurs for example, when the nucleotide complexities of random primed compared particular gene synthesized LPN molecule populations are different. The same LPN nucleotide sequences, refers to a population average nucleotide sequence and sequence distribution which represents the particular gene LPNs.

Design Solution 21, 29.

Comparing undegraded LPNs occurs very infrequently.

Design Solution 22.

This also occurs infrequently.

Design Solution 30.

Here, the assay value for each UNF is measured and known to equal one.

Design Solution 32.

This condition occurs for most microarray assays where the compared LPN nucleotide lengths are not adjusted to much smaller nucleotide lengths.

Design Solution 33.

Prior art does not use such microarrays. For prior art cDNA microarrays the shortest particular gene mRNAs have a nucleotide length of roughly 200-300 nucleotides.

Design Solution 34.

This will minimize or eliminate the occurrence of NF related false negative results and their associated RDMs.

Relative to prior art normalization practice, the normalization of microarray measured particular gene comparison results is improved when one or more particular gene comparison RASR values produced by a microarray assay, is known to be validly normalized for one or more of the following. (i) one or more pertinent UNFs. (ii) one or more pertinent CNFs. (iii) one or more pertinent UNFs and one or more pertinent CNFs. (iv) one or more pertinent UNFs and all pertinent CNFs. (v) all pertinent CNFs. (vi) all pertinent UNFs. (vii) all pertinent UNFs and all pertinent CNFs.

For a microarray assay the preferred improved normalization process assay design solution combination results in the valid normalization of all particular gene comparison RASR values in an assay for all pertinent UNFs and CNFs, and also results in minimizing the number of UNF and CNF related false negative results which are associated with the assay. Such assay designs are described below. A variety of different general assay designs are practiced by the prior art, and each of these different general assay designs can be associated with a different combination of pertinent UNFs and CNFs. Certain of these prior art general assay designs are associated with pertinent UNFs, such as the PSSR and PAFR, whose assay values cannot practically be determined for each particular gene comparison in an assay, or with pertinent UNFs, such as the PL-HKR and PS-HKR, whose assay values cannot currently be determined due to lack of information which is currently unknown, but obtainable. Therefore, some prior art general assay designs cannot be modified to allow the improved normalization for all pertinent UNFs and CNFs. This is discussed below. For simplicity, each different prior art general assay design will be discussed in terms of the Table 53 design solution combinations which can be known to allow the improved normalization of all or essentially all particular gene comparison RASR values in the assay for the maximum number of pertinent UNFs and CNFs. These preferred practice design solution combinations are presented in Tables 54 through 60.

TABLE 54
Preferred Practice for Design Solution Combinations Which Can Be Known to
Completely Normalize All, or Essentially All, Microarray Assay Particular
Gene RASR Values for All Pertinent UNFs and CNFs: Comparison of
Cell Sample Directly Labeled mRNAs Produced from T-RNAs

| | | NFs Which | | Pertinent | |
|---|---|---|---|---|---|
| | | Can Be | | NFs To Be | |
| | | Ignored For | | Determined and | |
| | | Normalization | | Normalized For | |
| Combination of Assay Design Solutions | | UNF | CNF | UNF | CNF |
| | Compare Undegraded T-RNA Type 1 | PSSR | C-HKR | SCR | Spatial |
| | mRNA LPNs | PAFR | | PSAR | Print Tip |
| (1) | Combine Table 53 Design Solutions | MLDR | | | Print Plate |
| | (a) 1, or 2, or 3, 4a, 5a, 6, 8a, c, | PL-HKR | | | Intensity |
| | 13b, 14, 22a, 27, 29, 30a, b, 34 | PS-HKR | | | Scale |
| | or | LLSR | | | |
| | (b) 1, or 2, or 3, 4b, 5a, 6, 8a, c, | PSAR | | | |
| | 13b, 14, 22a, 27, 29, 30a, b, 34 | SCR | | | |
| | or | | | | |
| | (c) As (1a) or (1b), except use Design | | | | |
| | Solution 7 instead of Design | | | | |
| | Solution 6 | | | | |
| | or | | | | |
| | (d) As (1a-c), except delete Design | | | | |
| | Solutions 8a, c | | | | |
| | or | | | | |
| | (e) As (1a-d), except use Design | | | | |
| | Solution 25b | | | | |
| (2) | Combine Table 53 Design Solutions | PSSR | C-HKR | SCR | Spatial |
| | (a) As (1a-e), except delete Design | PAFR | | PSAR | Print Tip |
| | Solution, 30a, b | MLDR | | | Print Plate |
| | | PL-HKR | | | Intensity |
| | | PS-HKR | | | Scale |
| | | LLSR | | | |
| (3) | Combine Table 53 Design Solutions | PSSR | — | SCR | C-HKR |
| | (a) As (1a-e), except use Design | PAFR | | PSAR | Spatial |
| | Solution, 13a instead | MLDR | | | Print Tip |
| | of Design Solution 13b | PL-HKR | | | Print Plate |
| | | PS-HKR | | | Intensity |
| | | LLSR | | | Scale |
| | | SCR | | | |
| | | PSAR | | | |
| (4) | Combine Table 53 Design Solutions | PSSR | — | SCR | C-HKR |
| | (a) As (2a), except use Design | PAFR | | PSAR | Spatial |
| | Solution 13a instead of Design | MLDR | | | Print Tip |
| | Solution 13b. | PL-HKR | | | Print Plate |
| | | PS-HKR | | | Intensity |
| | | LLSR | | | Scale |
| | Compare Degraded T-RNA Type 1 | PSSR | C-HKR | SCR | Spatial |
| | mRNA LPNs | PAFR | | PSAR | Print Tip |
| (5) | Combine Table 53 Design Solutions | | | | |
| | (a) 1, or 2, or 3, 4a, 5a, 6, 8a, c, 13b, | MLDR | | | Print Plate |
| | 14, 22a, 23b, 24a, | PL-HKR | | | Intensity |
| | 26a, 27, 30a, b, 34 | PS-HKR | | | Scale |
| | or | LLSR | | | |
| | (b) 1, or 2, or 3, 4b, 5a, 6, 8a, c, | SCR | | | |
| | 13b, 14, 22a, 23b, 24a, | PSAR | | | |
| | 26a, 27, 30a, b, 34 | | | | |
| | or | | | | |
| | (c) As (5a) or (5b), except use Design | | | | |
| | Solution 7 | | | | |
| | instead of Design Solution 6 | | | | |
| | or | | | | |
| | (d) As (5a-c), except delete Design | | | | |
| | Solution 8a, c | | | | |
| | or | | | | |
| | (e) As (5a-d), except delete Design | | | | |
| | Solutions 24a and | | | | |
| | 26a and use Design Solutions 24b, | | | | |
| | 26b, and 25a | | | | |
| | or | | | | |
| | (f) As (5a-e), except also use Design | | | | |
| | Solution 25b | | | | |
| (6) | Combine Table 53 Design Solutions | PSSR | C-HKR | SCR | Spatial |
| | (a) As (5a-f), except delete Design | PAFR | | PSAR | Print Tip |
| | Solution 30a, b | MLDR | | | Print Plate |
| | | PL-HKR | | | Intensity |
| | | PS-HKR | | | Scale |
| | | LLSR | | | |
| (7) | Combine Table 53 Design Solutions | PSSR | — | SCR | C-HKR |
| | (a) As (5a-f), except use Design | PAFR | | PSAR | Spatial |
| | Solution 13a instead of 13b | MLDR | | | Print Tip |
| | | PL-HKR | | | Print Plate |
| | | PS-HKR | | | Intensity |
| | | LLSR | | | Scale |
| | | SCR | | | |
| | | PSAR | | | |
| (8) | Combine Table 53 Design Solutions | PSSR | — | SCR | C-HKR |
| | (a) As (6a), except use Design | PAFR | | PSAR | Spatial |
| | Solution 13a instead of 13b | MLDR | | | Print Tip |
| | | PL-HKR | | | Print Plate |
| | | PS-HKR | | | Intensity |
| | | LLSR | | | Scale |
| | Compare Undegraded T-RNA Type 2 | PSSR | C-HKR | SCR | Spatial |
| | mRNA LPNs | PAFR | | LLSR | Print Tip |
| (9) | Combine Table 53 Design Solutions | MLDR | | | Print Plate |
| | (a) 1, or 2, or 3, 4a, 5b, 6, 8a, b, | PL-HKR | | | Intensity |
| | 13b, 14, 22a, 27, 29, 30b, c, 34 | PS-HKR | | | Scale |
| | or | PSAR | | | |
| | (b) 1, or 2, or 3, 4b, 5b, 6, 8a, b, | SCR | | | |
| | 13b, 14, 22a, 27, 29, 30b, c, 34 | | | | |
| | or | | | | |
| | (c) As (9a-b), except use Design | LLSR | | | |
| | Solution 7 instead | | | | |
| | of Design Solution 6 | | | | |
| | or | | | | |
| | (d) As (9a-c), except delete Design | | | | |
| | Solution 8a, b | | | | |
| | or | | | | |
| | (e) As (9a-d), except use Design | | | | |
| | Solution 25b | | | | |
| (10) | Combine Table 53 Design Solutions | PSSR | C-HKR | SCR | Spatial |
| | (a) As (9a-e), except delete Design | PAFR | | LLSR | Print Tip |
| | Solution 30b, c | MLDR | | | Print Plate |
| | | PL-HKR | | | Intensity |
| | | PS-HKR | | | Scale |
| | | PSAR | | | |
| (11) | Combine Table 53 Design Solutions | PSSR | — | SCR | C-HKR |
| | (a) As (9a-e), except use Design | PAFR | | LLSR | Spatial |
| | Solution 13a instead | MLDR | | | Print Tip |
| | of Design Solution 13b | PL-HKR | | | Print Plate |
| | | PS-HKR | | | Intensity |
| | | PSAR | | | Scale |
| | | SCR | | | |
| | | LLSR | | | |
| (12) | Combine Table 53 Design Solutions | PSSR | — | SCR | C-HKR |
| | (a) As (10a), except use Design | PAFR | | LLSR | Spatial |
| | Solution 13a instead | MLDR | | | Print Tip |
| | of Design Solution 13b | PL-HKR | | | Print Plate |
| | | PS-HKR | | | Intensity |
| | | PSAR | | | Scale |
| | Compare Degraded T-RNA Type 2 | PSSR | C-HKR | SCR | Spatial |
| | mRNA LPNs | PAFR | | LLSR | Print Tip |
| (13) | Combine Table 53 Design Solutions | MLDR | | | Print Plate |
| | (a) 1, or 2, or 3, 4a, 5b, 6, 8a, b, | PL-HKR | | | Intensity |
| | 13b, 14, 22a, 23b, 24a, 26a, 27, | PS-HKR | | | Scale |
| | 30b, c, 34 | PSAR | | | |
| | or | | | | |
| | (b) 1, or 2, or 3, 4b, 5b, 6, 8a, b, | SCR | | | |
| | 13b, 14, 22a, 23b, 24a, 26a, 27, | LLSR | | | |
| | 30b, c, 34 | | | | |
| | or | | | | |
| | (c) As (13a) or (13b), except use | | | | |
| | Design Solution 7 | | | | |
| | instead of Design Solution 6 | | | | |
| | or | | | | |
| | (d) As (13a-c), except delete Design | | | | |
| | Solutions 8a, b | | | | |
| | or | | | | |
| | (e) As (13a-d), except delete Design | | | | |
| | Solution 24a and 26a, and use Design | | | | |
| | Solutions 24b, 26b, and 25a | | | | |
| | or | | | | |
| | (f) As (13a-e), except also use | | | | |
| | Design Solution 25b | | | | |
| (14) | Combine Table 53 Design Solutions | PSSR | C-HKR | SCR | Spatial |
| | (a) As (13a-f), except use Design | PAFR | | LLSR | Print Tip |
| | Solution 30b, c | MLDR | | | Print Plate |
| | | PL-HKR | | | Intensity |
| | | PS-HKR | | | Scale |
| | | PSAR | | | |
| (15) | Combine Table 53 Design Solutions | PSSR | — | SCR | C-HKR |
| | (a) As (13a-f), except use Design | PAFR | | LLSR | Spatial |
| | Solution 13a instead of | MLDR | | | Print Tip |
| | Design Solution 13b | PL-HKR | | | Print Plate |
| | | PS-HKR | | | Intensity |
| | | PSAR | | | Scale |
| | | SCR | | | |
| | | LLSR | | | |
| (16) | Combine Table 53 Design Solutions | PSSR | — | SCR | C-HKR |
| | (a) As (14a), except use Design | PAFR | | LLSR | Spatial |
| | Solution 13a instead of | MLDR | | | Print Tip |
| | Design Solution 13b | PL-HKR | | | Print Plate |
| | | PS-HKR | | | Intensity |
| | | PSAR | | | Scale |
| (17) | For a degraded Type 2 mRNA LPN | | | | |
| | prep. Only one fragment from each | | | | |
| | formerly undegraded mRNA | | | | |
| | molecule is associated with a label. | | | | |
| | As an example, only each 3′ poly A | | | | |
| | tract containing mRNA fragment, or | | | | |
| | each 5′ cap containing fragment in | | | | |
| | the T-RNA prep, is associated with a | | | | |
| | label. | | | | |

TABLE 55
Preferred Practices for Design Solution Combinations Which Can Be Known to
Completely Normalize All, or Essentially All, Microarray Assay Particular Gene
RASR Values for All Pertinent UNFs and CNFs: Comparison of Specific Gene (SG)
Primed Directly Labeled LPNs Produced from T-RNAs

| | | NFs Which | | Pertinent | |
|---|---|---|---|---|---|
| | | Can Be | | NFs To Be | |
| | | Ignored For | | Determined and | |
| | | Normalization | | Normalized For | |
| Combination of Assay Design Solutions | | UNF | CNF | UNF | CNF |
| | Compare Undegraded T-RNA Type 1 | PSSR | C-HKR | SCR | Spatial |
| | LPNs From T-RNA | PAFR | | PSAR | Print Tip |
| (1) | Combine Table 53 Design Solutions | MLDR | | | Print Plate |
| | (a) 1, or 2, or 3, 4a, 5a, 6, 8a, c, 10a, | PL-HKR | | | Intensity |
| | 13b, 14, 19, 21, 30a, b, 34 | PS-HKR | | | Scale |
| | or | LLSR | | | |
| | (b) 1, or 2, or 3, 4b, 5a, 6, 8a, c, 10a, | SCR | | | |
| | 13b, 14, 19, 21, | PSAR | | | |
| | 30a, b, 34 | | | | |
| | or | | | | |
| | (c) As (1a) or (1b), except use Design | | | | |
| | Solution 7 | | | | |
| | instead of Design Solution 6. | | | | |
| | or | | | | |
| | (d) As (1a-c), except delete Design | | | | |
| | Solutions 8a, c | | | | |
| | or | | | | |
| | (e) As (1a-d), except use Design | | | | |
| | Solution 17b | | | | |
| (2) | Combine Table 53 Design Solutions | PSSR | C-HKR | SCR | Spatial |
| | (a) As (1a-e), except delete Design | PAFR | | PSAR | Print Tip |
| | Solution, 30a, b. | MLDR | | | Print Plate |
| | | PL-HKR | | | Intensity |
| | | PS-HKR | | | Scale |
| | | LLSR | | | |
| (3) | Combine Table 53 Design Solutions | PSSR | — | SCR | C-HKR |
| | (a) As (1a-e), except use Design | PAFR | | PSAR | Spatial |
| | Solution 13a instead of Design Solution | MLDR | | | Print Tip |
| | 13b | PL-HKR | | | Print Plate |
| | | PS-HKR | | | Intensity |
| | | LLSR | | | Scale |
| | | SCR | | | |
| | | PSAR | | | |
| (4) | Combine Table 53 Design Solutions | PSSR | — | SCR | C-HKR |
| | (a) As (2a), except use Design | PAFR | | PSAR | Spatial |
| | Solution 13a instead of | MLDR | | | Print Tip |
| | Design Solution 13b. | PL-HKR | | | Print Plate |
| | | PS-HKR | | | Intensity |
| | | LLSR | | | Scale |
| | Comparison of Type 1 LPNs From T-RNA | PSSR | C-HKR | SCR | Spatial |
| (5) | Combine Table 53 Design Solutions | PAFR | | PSAR | Print Tip |
| | (a) 1, or 2, or 3, 4a, 5a, 6, 8a, c, 10a, 13b, | MLDR | | | Print Plate |
| | 14, 15a, 16a, 18a, 19, 30a, b, 34. or | PL-HKR | | | Intensity |
| | (b) 1, or 2, or 3, 4b, 5a, 6, 8a, c, 10a, 13b, | PS-HKR | | | Scale |
| | 14, 15a, 16a, 18a, 19, 30a, b, 34, or | LLSR | | | |
| | (c) As (5a) or (5b), except use Design | SCR | | | |
| | Solution 15b instead of Design Solution | PSAR | | | |
| | 15a, or | | | | |
| | (d) As (5a-c), except use Design Solution | | | | |
| | 7 instead of Design Solution 6, or | | | | |
| | (e) As (5a-d), except delete Design | | | | |
| | Solutions 8a, c, or | | | | |
| | (f) As (5a-e), except delete Design | | | | |
| | Solutions 1, 3, 16a, and 18a, and use | | | | |
| | Design Solutions 16b, 18b, 17a, and 2 or | | | | |
| | 33, or | | | | |
| | (g) As (5a-f), except also use Design | | | | |
| | Solution 17b | | | | |
| (6) | Combine Table 53 Design Solutions | PSSR | C-HKR | SCR | Spatial |
| | (a) As (5a-g), except delete Design | PAFR | | PSAR | Print Tip |
| | Solution 30a, b | MLDR | | | Print Plate |
| | | PL-HKR | | | Intensity |
| | | PS-HKR | | | Scale |
| | | LLSR | | | |
| (7) | Combine Table 53 Design Solutions | PSSR | — | SCR | C-HKR |
| | (a) As (5a-g), except use Design | PAFR | | PSAR | Spatial |
| | Solution 13a instead of Design Solution | MLDR | | | Print Tip |
| | 13b | PL-HKR | | | Print Plate |
| | | PS-HKR | | | Intensity |
| | | LLSR | | | Scale |
| | | SCR | | | |
| | | PSAR | | | |
| (8) | Combine Table 53 Design Solutions | PSSR | — | SCR | C-HKR |
| | (a) As (6a), except use Design | PAFR | | PSAR | Spatial |
| | Solution 13a instead | MLDR | | | Print Tip |
| | of Design Solution 13b | PL-HKR | | | Print Plate |
| | | PS-HKR | | | Intensity |
| | | LLSR | | | Scale |
| | Comparison of Undegraded T-RNA Type | PSSR | C-HKR | SCR | Spatial |
| | 2 LPNs | PAFR | | LLSR | Print Tip |
| (9) | Combine Table 53 Design Solutions | MLDR | | | Print Plate |
| | (a) 1, or 2, or 3, 4a, 5b, 6, 8a, b, 10a, 13b, | PL-HKR | | | Intensity |
| | 14, 18a, 19, 21, 30b, c, 34 | PS-HKR | | | Scale |
| | or | PSAR | | | |
| | (b) 1, or 2, or 3, 4b, 5b, 6, 8a, b, 10a, 13b, | SCR | | | |
| | 14, 18a, 19 21, 30a, b, 34 | LLSR | | | |
| | or | | | | |
| | (c) As (9a) or (9b), except use Design | | | | |
| | Solution 7 instead of Design Solution 6 | | | | |
| | or | | | | |
| | (d) As (9a-c), except delete Design | | | | |
| | Solution 8a, b | | | | |
| | or | | | | |
| | (e) As (9a-d), except use Design Solution | | | | |
| | 17b | | | | |
| (10) | Combine Table 53 Design Solutions | PSSR | C-HKR | SCR | Spatial |
| | (a) As (9a-e), except delete Design | PAFR | | LLSR | Print Tip |
| | Solution 30b, c | MLDR | | | Print Plate |
| | | PL-HKR | | | Intensity |
| | | PS-HKR | | | Scale |
| | | PSAR | | | |
| (11) | Combine Table 53 Design Solutions | PSSR | — | SCR | C-HKR |
| | (a) As (9a-e), except use Design | PAFR | | LLSR | Spatial |
| | Solution 13a instead | MLDR | | | Print Tip |
| | of Design Solution 13b | PL-HKR | | | Print Plate |
| | | PS-HKR | | | Intensity |
| | | PSAR | | | Scale |
| | | SCR | | | |
| | | LLSR | | | |
| (12) | Combine Table 53 Design Solutions | PSSR | — | SCR | C-HKR |
| | (a) As (10a), except use Design | PAFR | | LLSR | Spatial |
| | Solution 13a instead | MLDR | | | Print Tip |
| | of Design Solution 13b | PL-HKR | | | Print Plate |
| | | PS-HKR | | | Intensity |
| | | PSAR | | | Scale |
| | Comparison of Type 2 LPNs Produced | PSSR | C-HKR | SCR | Spatial |
| | From T-RNAs | PAFR | | LLSR | Print Tip |
| (13) | Combine Table 53 Design Solutions | MLDR | | | Print Plate |
| | (a) 1, or 2, or 3, 4a, 5b, 6, 8a, b, 10a, | PL-HKR | | | Intensity |
| | 13b, 14, 15a, 16a, 18a, 19, 30b, | PS-HKR | | | Scale |
| | c, 34 | PSAR | | | |
| | or | SCR | | | |
| | (b) 1, or 2, or 3, 4b, 5b, 6, 8a, b, 10a, | LLSR | | | |
| | 13b, 14, 15a, 16a, 18a, 19, 30b, c, | | | | |
| | 34 | | | | |
| | or | | | | |
| | (c) As (13a) or (13b), except use | | | | |
| | Design Solution 15b | | | | |
| | instead of Design Solution 15a | | | | |
| | or | | | | |
| | (d) As (13a-c), except use Design | | | | |
| | Solutions 7 instead of Design | | | | |
| | Solution 6 | | | | |
| | or | | | | |
| | (e) As (13a-d), except delete Design | | | | |
| | Solution 8a, b | | | | |
| | or | | | | |
| | (f) As (13a-d), except delete Design | | | | |
| | Solutions 16a and 18a, and use | | | | |
| | Design Solutions 16b, 18b, and | | | | |
| | 17a | | | | |
| | or | | | | |
| | (g) As (13a-f), except also use | | | | |
| | Design Solution 17b | | | | |
| (14) | Combine Table 53 Design Solutions | PSSR | C-HKR | SCR | Spatial |
| | (a) As (13a-g), except use Design | PAFR | | LLSR | Print Tip |
| | Solutions 30b, c | MLDR | | | Print Plate |
| | | PL-HKR | | | Intensity |
| | | PS-HKR | | | Scale |
| | | PSAR | | | |
| (15) | Combine Table 53 Design | PSSR | — | SCR | C-HKR |
| | Solutions | PAFR | | LLSR | Spatial |
| | (a) As (13a-g), except use Design | MLDR | | | Print Tip |
| | Solution 13a instead of Design | PL-HKR | | | Print Plate |
| | Solution 13b | PS-HKR | | | Intensity |
| | | PSAR | | | Scale |
| | | SCR | | | |
| | | LLSR | | | |
| (16) | Combine Table 53 Design | PSSR | — | SCR | C-HKR |
| | Solutions | PAFR | | LLSR | Spatial |
| | (a) As (14a), except use Design | MLDR | | | Print Tip |
| | Solution 13a instead of Design | PL-HKR | | | Print Plate |
| | Solution 13b | PS-HKR | | | Intensity |
| | | PSAR | | | Scale |

TABLE 56
Preferred Practices for Design Solution Combinations Which Can Be Known to
Completely Normalize All, or Essentially All, Microarray Assay Particular Gene
RASR Values for All Pertinent UNFs and CNFs: Comparison of Random Primed
Directly Labeled LPNs Produced from T-RNA

| | | NFs Which | | Pertinent | |
|---|---|---|---|---|---|
| | | Can Be | | NFs To Be | |
| | | Ignored For | | Determined and | |
| | | Normalization | | Normalized For | |
| Combination of Assay Design Solutions | | UNF | CNF | UNF | CNF |
| | Compare Type 1 LPNs | PSSR | C-HKR | SCR | Spatial |
| (1) | Combine Table 53 Design Solutions | PAFR | | PSAR | Print Tip |
| | (a) 1, or 2, or 3, 4a, 5a, 6, 8a, c, 11, | MLDR | | | Print Plate |
| | 13b, 14, 15b, 16a, 18a, 19, 30a, b, | PL-HKR | | | Intensity |
| | 34, or | PS-HKR | | | Scale |
| | (b) 1, or 2, or 3, 4b, 5a, 6, 8a, c, 11, | LLSR | | | |
| | 13b, 14, 15b, 16a, 18a, 19, 30a, b, | SCR | | | |
| | 34, or | PSAR | | | |
| | (c) As (1a) or (1b), except use | | | | |
| | Design Solution 7 instead of | | | | |
| | Design Solution 6, or | | | | |
| | (d) As (1a-c), except delete Design | | | | |
| | Solutions 8a, c, or | | | | |
| | (e) As (1a-d), except delete Design | | | | |
| | Solutions 16a and 18a and use | | | | |
| | Design Solutions 16b, 18b, and | | | | |
| | 17a, or | | | | |
| | (f) As (1a-e), except also use Design | | | | |
| | Solution 17b | | | | |
| (2) | Combine Table 53 Design Solutions | PSSR | C-HKR | SCR | Spatial |
| | (a) As (1a-f), except delete Design | PAFR | | PSAR | Print Tip |
| | Solution, 30a, b | MLDR | | | Print Plate |
| | | PL-HKR | | | Intensity |
| | | PS-HKR | | | Scale |
| | | LLSR | | | |
| (3) | Combine Table 53 Design Solutions | PSSR | — | SCR | C-HKR |
| | (a) As (1a-f), except use Design | PAFR | | PSAR | Spatial |
| | Solution 13a instead of Design | MLDR | | | Print Tip |
| | Solution 13b | PL-HKR | | | Print Plate |
| | | PS-HKR | | | Intensity |
| | | LLSR | | | Scale |
| | | SCR | | | |
| | | PSAR | | | |
| (4) | Combine Table 53 Design Solutions | PSSR | — | SCR | C-HKR |
| | (a) As (2a), except use Design | PAFR | | PSAR | Spatial |
| | Solution 13a instead of Design | MLDR | | | Print Tip |
| | Solution 13b. | PL-HKR | | | Print Plate |
| | | PS-HKR | | | Intensity |
| | | LLSR | | | Scale |

TABLE 57
Preferred Practices for Design Solution Combinations Which Can Be Known to
Provide Improved Normalization for Pertinent UNFs and/or CNFs for All or
Essentially All Microarray Measured Particular Gene RASR Values in an Assay:
Comparison of Oligo dT Primed Directly Labeled L-LPNs Produced from T-RNAs or
Isolated mRNAs

| | | NFs Which | | Pertinent | |
|---|---|---|---|---|---|
| | | Can Be | | NFs To Be | |
| | | Ignored For | | Determined and | |
| | | Normalization | | Normalized For | |
| Combination of Assay Design Solutions | | UNF | CNF | UNF | CNF |
| | Compare Undegraded RNA Type 1 LPNs | PSSR | C-HKR | PAFR | Spatial |
| (1) | Combine Table 53 Design Solutions | MLDR | | SCR | Print Tip |
| | (a) 1, or 2, or 3, 4a, 5a, 6, 8a, c, 9a or | PL-HKR | | PSAR | Print Plate |
| | b, 13b, 14, 18a, 19, 21, 30a, b, 34 | PS-HKR | | | Intensity |
| | or | LLSR | | | Scale |
| | (b) 1, or 2, or 3, 4b, 5a, 6, 8a, c, 9a or | SCR | | | |
| | b, 13b, 14, 18a, 19, 21, 30a, b, 34 | PSAR | | | |
| | or | | | | |
| | (c) As (1a-b), except use Design | | | | |
| | Solution 7 instead of Design | | | | |
| | Solution 6. | | | | |
| | or | | | | |
| | (d) As (1a-c), except delete Design | | | | |
| | Solutions 8a, c | | | | |
| | or | | | | |
| | (e) As (1a-d), except use Design | | | | |
| | Solution 17b | | | | |
| (2) | Combine Table 53 Design Solutions | PSSR | C-HKR | PAFR | Spatial |
| | (a) As (1a-e), except delete Design | MLDR | | SCR | Print Tip |
| | Solution, 30a, b | PL-HKR | | PSAR | Print Plate |
| | | PS-HKR | | | Intensity |
| | | LLSR | | | Scale |
| (3) | Combine Table 53 Design Solutions | PSSR | — | PAFR | C-HKR |
| | (a) As (1a-e), except use Design | MLDR | | SCR | Spatial |
| | Solution 13a instead of Design | PL-HKR | | PSAR | Print Tip |
| | Solution 13b | PS-HKR | | | Print Plate |
| | | LLSR | | | Intensity |
| | | SCR | | | Scale |
| | | PSAR | | | |
| (4) | Combine Table 53 Design Solutions | PSSR | — | PAFR | C-HKR |
| | (a) As (2a), except use Design | MLDR | | SCR | Spatial |
| | Solution 13a instead of Design | PL-HKR | | PSAR | Print Tip |
| | Solution 13b. | PS-HKR | | | Print Plate |
| | | LLSR | | | Intensity |
| | | | | | Scale |
| | Comparison of Type 1 LPNs From T- | PSSR | C-HKR | PAFR | Spatial |
| | RNA or Isolated mRNA | MLDR | | SCR | Print Tip |
| (5) | Combine Table 53 Design Solutions | PL-HKR | | PSAR | Print Plate |
| | (a) 1, or 2, or 3, 4a, 5a, 6, 8a, c, 9a or | PS-HKR | | | Intensity |
| | b, 13b, 14, 15a, 16a, 18a, 19, 30a, | LLSR | | | Scale |
| | b, 34, or | SCR | | | |
| | (b) 1, or 2, or 3, 4b, 5a, 6, 8a, c, 9a or | PSAR | | | |
| | b, 13b, 14, 15a, 16a, 18a, 19, 30a, | | | | |
| | b, or | | | | |
| | (c) As (5a-b), except use Design | | | | |
| | Solution 15b instead of Design | | | | |
| | Solution 15a, or | | | | |
| | (d) As (5a-c), except use Design | | | | |
| | Solution 7 instead of Design | | | | |
| | Solution 6, or | | | | |
| | (e) As (5a-d), except delete Design | | | | |
| | Solutions 8a, c, or | | | | |
| | (f) As (5a-e), except delete Design | | | | |
| | Solutions 1, 3, 16a, and 18a and | | | | |
| | use Design Solutions 16b, 18b, | | | | |
| | 17a, and 2 or 33, or | | | | |
| | (g) As (5a-f), except also use Design | | | | |
| | Solution 17b | | | | |
| (6) | Combine Table 53 Design Solutions | PSSR | C-HKR | PAFR | Spatial |
| | (a) As (5a-g), except delete Design | MLDR | | SCR | Print Tip |
| | Solutions 30a, b | PL-HKR | | PSAR | Print Plate |
| | | PS-HKR | | | Intensity |
| | | LLSR | | | Scale |
| (7) | Combine Table 53 Design Solutions | PSSR | — | PAFR | C-HKR |
| | (a) As (5a-g), except use Design | MLDR | | SCR | Spatial |
| | Solution 13a instead of Design | PL-HKR | | PSAR | Print Tip |
| | Solution 13b | PS-HKR | | | Print Plate |
| | | LLSR | | | Intensity |
| | | SCR | | | Scale |
| | | PSAR | | | |
| (8) | Combine Table 53 Design Solutions | PSSR | — | PAFR | C-HKR |
| | (a) As (6a), except use Design | MLDR | | SCR | Spatial |
| | Solution 13a instead of Design | PL-HKR | | PSAR | Print Tip |
| | Solution 13b | PS-HKR | | | Print Plate |
| | | LLSR | | | Intensity |
| | | | | | Scale |
| | Comparison of Undegraded RNA Type 2 | PSSR | C-HKR | PAFR | Spatial |
| | LPNs | MLDR | | SCR | Print Tip |
| (9) | Combine Table 53 Design Solutions | PL-HKR | | LLSR | Print Plate |
| | (a) 1, or 2, or 3, 4a, 5b, 6, 8a, b, 9a or b, | PS-HKR | | | Intensity |
| | 13b, 14, 18a, 19, 21, 30b, c, 34 | PSAR | | | Scale |
| | or | SCR | | | |
| | (b) 1, or 2, or 3, 4b, 5b, 6, 8a, b, 9a or b, | LLSR | | | |
| | 13b, 14, 18a, 19, 21, 30b, c, 34 | | | | |
| | or | | | | |
| | (c) As (9a-b), except use Design Solution | | | | |
| | 7 instead of Design Solution 6 | | | | |
| | or | | | | |
| | (d) As (9a-c), except delete Design | | | | |
| | Solution 8a, b | | | | |
| | or | | | | |
| | (e) As (9a-d), except use Design Solution | | | | |
| | 17b | | | | |
| (10) | Combine Table 53 Design Solutions | PSSR | C-HKR | PAFR | Spatial |
| | (a) As (9a-e), except delete Design | MLDR | | SCR | Print Tip |
| | Solution 30b, c | PL-HKR | | LLSR | Print Plate |
| | | PS-HKR | | | Intensity |
| | | PSAR | | | Scale |
| (11) | Combine Table 53 Design Solutions | PSSR | — | PAFR | C-HKR |
| | (a) As (9a-f), except use Design | MLDR | | SCR | Print Tip |
| | Solution 13a instead of Design | PL-HKR | | LLSR | Print Plate |
| | Solution 13b | PS-HKR | | | Intensity |
| | | PSAR | | | Scale |
| | | SCR | | | |
| | | LLSR | | | |
| (12) | Combine Table 53 Design Solutions | PSSR | — | PAFR | C-HKR |
| | (a) As (10a), except use Design | MLDR | | SCR | Spatial |
| | Solution 13a instead of Design | PL-HKR | | LLSR | Print Tip |
| | Solution 13b | PS-HKR | | | Print Plate |
| | | PSAR | | | Intensity |
| | | SCR | | | Scale |
| | | LLSR | | | |
| | Compare of Type 2 L-LPNs Produced | PSSR | C-HKR | PAFR | Spatial |
| | From T-RNA or Isolated mRNA | MLDR | | SCR | Print Tip |
| (13) | Combine Table 53 Design Solutions | PL-HKR | | LLSR | Print Plate |
| | (a) 1, or 2, or 3, 4a, 5b, 6, 8a, b, 9a | PS-HKR | | | Intensity |
| | or b, 13b, 14, 15a, 16a, 18a, 19, | PSAR | | | Scale |
| | 21, 34 | SCR | | | |
| | or | LLSR | | | |
| | (b) 1, or 2, or 3, 4b, 5b, 6, 8a, b, 9a | | | | |
| | or b, 13b, 14, 15a, 16a, 18a, 19, | | | | |
| | 21, 34 | | | | |
| | or | | | | |
| | (c) As (13a-b), except use Design | | | | |
| | Solution 15b instead of Design | | | | |
| | Solution 15a | | | | |
| | or | | | | |
| | (d) As (13a-c), except use Design | | | | |
| | Solutions 7 instead of Design | | | | |
| | Solution 6 | | | | |
| | or | | | | |
| | (e) As (13a-d), except delete | | | | |
| | Design Solution 8a, b | | | | |
| | or | | | | |
| | (f) As (13a-e), except delete Design | | | | |
| | Solutions 16a and 18a, and use | | | | |
| | Design Solutions 16b, 18b, and | | | | |
| | 17a | | | | |
| | or | | | | |
| | (g) As (13a-f), except also use | | | | |
| | Design Solution 17b | | | | |
| (14) | Combine Table 53 Design Solutions | PSSR | C-HKR | PAFR | Spatial |
| | (a) As (13a-g), except delete | MLDR | | SCR | Print Tip |
| | Design Solutions 30b, c | PL-HKR | | LLSR | Print Plate |
| | | PS-HKR | | | Intensity |
| | | PSAR | | | Scale |
| (15) | Combine Table 53 Design Solutions | PSSR | — | PAFR | C-HKR |
| | (a) As (13a-g), except use Design | MLDR | | SCR | Spatial |
| | Solution 13a instead of Design | PL-HKR | | LLSR | Print Tip |
| | Solution 13b | PS-HKR | | | Print Plate |
| | | PSAR | | | Intensity |
| | | SCR | | | Scale |
| | | LLSR | | | |
| (16) | Combine Table 53 Design Solutions | PSSR | — | PAFR | C-HKR |
| | (a) As (14a), except use Design | MLDR | | SCR | Spatial |
| | Solution 13a instead of Design | PL-HKR | | LLSR | Print Tip |
| | Solution 13b | PS-HKR | | | Print Plate |
| | | PSAR | | | Intensity |
| | | | | | Scale |

TABLE 58
Preferred Practices for Design Solution Combinations Which Can Be Known to
Produce, Relative to the Prior Art, Improved Normalization for Pertinent UNFs
and/or CNFs, for All Microarray Measured Particular Gene RASR Values in an
Assay: Comparison of Directly Labeled Isolated mRNA LPNs

| | | NFs | | Pertinent NFs | |
|---|---|---|---|---|---|
| | | Which Can Be | | To Be | |
| | | Ignored For | | Determined and | |
| | | Normalization | | Normalized For | |
| Combination of Assay Design Solutions | | UNF | CNF | UNF | CNF |
| | Compare Undegraded Isolated | PSSR | C-HKR | PAFR | Spatial |
| | mRNA Type 1 LPNs | MLDR | | SCR | Print Tip |
| (1) | Combine Table 53 Design Solutions | PL-HKR | | SAR | Print Plate |
| | (a) 1, or 2, or 3, 4a, 5a, 6, 8a, c, 13b, | PS-HKR | | | Intensity |
| | 14, 22b, 27, 29, 30a, b, 34 | LLSR | | | Scale |
| | or | SCR | | | |
| | (b) 1, or 2, or 3, 4b, 5a, 6, 8a, c, 13b, | PSAR | | | |
| | 14, 22, b, 27, 29, | | | | |
| | 30a, b, 34 | | | | |
| | or | | | | |
| | (c) As (1a-b), except use Design | | | | |
| | Solution 7 instead of Design | | | | |
| | Solution 6 | | | | |
| | or | | | | |
| | (d) As (1a-c), except delete Design | | | | |
| | Solutions 8a, c | | | | |
| | or | | | | |
| | (e) As (1a-d), except use Design | | | | |
| | Solution 25b | | | | |
| (2) | Combine Table 53 Design Solutions | PSSR | C-HKR | PAFR | Spatial |
| | (a) As (1a-e), except delete Design | MLDR | | SCR | Print Tip |
| | Solution, 30a, b | PL-HKR | | PSAR | Print Plate |
| | | PS-HKR | | | Intensity |
| | | LLSR | | | Scale |
| (3) | Combine Table 53 Design Solutions | PSSR | — | PAFR | C-HKR |
| | (a) As (1a-e), except use Design | MLDR | | SCR | Spatial |
| | Solution 13a instead of Design | PL-HKR | | PSAR | Print Tip |
| | Solution 13b. | PS-HKR | | | Print Plate |
| | | LLSR | | | Intensity |
| | | SCR | | | Scale |
| | | PSAR | | | |
| (4) | Combine Table 53 Design Solutions | PSSR | — | PAFR | C-HKR |
| | (a) As (2a), except use Design | MLDR | | SCR | Spatial |
| | Solution 13a instead of Design | PL-HKR | | PSAR | Print Tip |
| | Solution 13b | PS-HKR | | | Print Plate |
| | | LLSR | | | Intensity |
| | | | | | Scale |
| | Compare Isolated mRNAs Which | PSSR | C-HKR | PAFR | Spatial |
| | Were Degraded Before Isolation | MLDR | | SCR | Print Tip |
| | From T-RNA: Type 1 LPNs | PL-HKR | | PSAR | Print Plate |
| (5) | Combine Table 53 Design Solutions | PS-HKR | | | Intensity |
| | (a) 1, or 2, or 3, 4a, 5a, 6, 8a, c, 13b, | LLSR | | | Scale |
| | 14, 22b, 23b, 24a, 26a, 27, 30a, b, | SCR | | | |
| | 34 | PSAR | | | |
| | or | | | | |
| | (b) 1, or 2, or 3, 4b, 5a, 6, 8a, c, 13b, | | | | |
| | 14, 22, b, 23b, 24a, 26a, 27, 30a, | | | | |
| | b, 34 | | | | |
| | or | | | | |
| | (c) As (5a-b), except use Design | | | | |
| | Solution 7 instead of Design | | | | |
| | Solution 6 | | | | |
| | or | | | | |
| | (d) As (5a-c), except delete Design | | | | |
| | Solution 8a, c | | | | |
| | or | | | | |
| | (e) As (5a-d), except delete Design | | | | |
| | Solutions 1, 3, 24a, and 26a, and | | | | |
| | use Design Solutions 24b, 26b, | | | | |
| | 25a, and 2 or 33 | | | | |
| | or | | | | |
| | (f) As (5a-e), except also use Design | | | | |
| | Solution 25b | | | | |
| (6) | Combine Table 53 Design Solutions | PSSR | C-HKR | PAFR | Spatial |
| | (a) As (5a-f), except delete Design | MLDR | | SCR | Print Tip |
| | Solution 30a, b | PL-HKR | | PSAR | Print Plate |
| | | PS-HKR | | | Intensity |
| | | LLSR | | | Scale |
| (7) | Combine Table 53 Design Solutions | PSSR | — | PAFR | Spatial |
| | (a) As (5a-f), except use Design | MLDR | | SCR | Print Tip |
| | Solution 13a instead of Design | PL-HKR | | PSAR | Print Plate |
| | Solution 13b | PS-HKR | | | Intensity |
| | | LLSR | | | Scale |
| | | SCR | | | |
| | | PSAR | | | |
| (8) | Combine Table 53 Design Solutions | PSSR | — | PAFR | C-HKR |
| | (a) As (6a), except use Design | MLDR | | SCR | Spatial |
| | Solution 13a instead of Design | PL-HKR | | PSAR | Print Tip |
| | Solution 13b | PS-HKR | | | Print Plate |
| | | LLSR | | | Intensity |
| | | | | | Scale |
| (9) | Combinations (5)-(8) are associated | — | — | — | — |
| | with mRNA which is isolated from | | | | |
| | degraded cell sample T-RNA. Here, | | | | |
| | only the 3′ Poly A associated portion | | | | |
| | of the mRNA will be present in the | | | | |
| | isolated mRNA. | | | | |
| | Compare Isolated mRNAs Which | PSSR | C-HKR | PAFR | Spatial |
| | Were Undegraded When Isolated, but | MLDR | | SCR | Print Tip |
| | Subsequently Became Degraded: | PL-HKR | | LLSR | Print Plate |
| | Type 1 LPNs | PS-HKR | | | Intensity |
| (10) | Combine Table 53 Design | LLSR | | | Scale |
| | Solutions | SCR | | | |
| | (a) As (5a-d and f) | PSAR | | | |
| (11) | Combine Table 53 Design Solutions | PSSR | C-HKR | PAFR | Spatial |
| | (a) As (5a-d), except delete Design | MLDR | | SCR | Print Tip |
| | Solution 30a, b | PL-HKR | | LLSR | Print Plate |
| | | PS-HKR | | | Intensity |
| | | LLSR | | | Scale |
| (12) | Combine Table 53 Design Solutions | PSSR | — | PAFR | C-HKR |
| | (a) As (5a-d and f), except use | MLDR | | SCR | Spatial |
| | Design Solution 13a instead of | PL-HKR | | PSAR | Print Tip |
| | Design Solution 13b | PS-HKR | | | Print Plate |
| | | LLSR | | | Intensity |
| | | SCR | | | Scale |
| | | PSAR | | | |
| (13) | Combine Table 53 Design Solutions | PSSR | — | PAFR | C-HKR |
| | (a) As (11a), except use Design | MLDR | | SCR | Spatial |
| | Solution 13a instead of Design | PL-HKR | | PSAR | Print Tip |
| | Solution 13b | PS-HKR | | | Print Plate |
| | | LLSR | | | Intensity |
| | | | | | Scale |
| | Compare Undegraded Isolated | PSSR | C-HKR | PAFR | Spatial |
| | mRNAs: Type 2 LPNs | MLDR | | SCR | Print Tip |
| (14) | Combine Table 53 Design Solutions | PL-HKR | | LLSR | Print Plate |
| | (a) 1, or 2, or 3, 4a, 5b, 6, 8a, b, | PS-HKR | | | Intensity |
| | 13b, 14, 22b, 26a, 27, 29 30b, | PSAR | | | Scale |
| | c, 34, or | SCR | | | |
| | (b) 1, or 2, or 3, 4b, 5b, 6, 8a, b, | LLSR | | | |
| | 13b, 14, 22b, 26a, 27, 29, 30b, | | | | |
| | c, 34, or | | | | |
| | (c) As (14a-b), except use Design | | | | |
| | Solution 7 instead of Design | | | | |
| | Solution 6, or | | | | |
| | (d) As (14a-c), except delete Design | | | | |
| | Solutions 8a, b, or | | | | |
| | (e) As (14a-d), except use Design | | | | |
| | Solution 25b | | | | |
| (15) | Combine Table 53 Design Solutions | PSSR | C-HKR | PAFR | Spatial |
| | (a) As (14a-e), except delete Design | MLDR | | SCR | Print Tip |
| | Solution 30b, c | PL-HKR | | LLSR | Print Plate |
| | | PS-HKR | | | Intensity |
| | | PSAR | | | Scale |
| (16) | Combine Table 53 Design Solutions | PSSR | — | PAFR | C-HKR |
| | (a) As (14a-e), except use Design | MLDR | | SCR | Spatial |
| | Solution 13a instead of Design | PL-HKR | | LLSR | Print Tip |
| | Solution 13b | PS-HKR | | | Print Plate |
| | | PSAR | | | Intensity |
| | | SCR | | | Scale |
| | | LLSR | | | |
| (17) | Combine Table 53 Design Solutions | PSSR | — | PAFR | C-HKR |
| | (a) As (15a), except use Design | MLDR | | SCR | Spatial |
| | Solution 13a instead of Design | PL-HKR | | LLSR | Print Tip |
| | Solution 13b | PS-HKR | | | Print Plate |
| | | PSAR | | | Intensity |
| | | | | | Scale |
| | Compare Degraded Isolated mRNA: | PSSR | C-HKR | PAFR | Spatial |
| | Type 2 LPN | MLDR | | SCR | Print Tip |
| (18) | Combine Table 53 Design Solutions | PL-HKR | | LSR | Print Plate |
| | (a) 1, or 2, or 3, 4a, 5b, 6, 8a, b, | PS-HKR | | | Intensity |
| | 13b, 14, 22a, 23b, 24a, 26a, 27, | PSAR | | | Scale |
| | 30b, c, 34, or | SCR | | | |
| | (b) 1, or 2, or 3, 4b, 5b, 6, 8a, b, | LLSR | | | |
| | 13b, 14, 22a, 23b, 24a, 26a, 27, | | | | |
| | 30b, c, 34, or | | | | |
| | (c) As (18a-b), except use Design | | | | |
| | Solution 7 instead of Design | | | | |
| | Solution 6, or | | | | |
| | (d) As (18a-c), except delete Design | | | | |
| | Solutions 8a, b, or | | | | |
| | (e) As (18a-d), except delete | | | | |
| | Design Solution 24a, and 26a, | | | | |
| | and use Design Solutions 24b, | | | | |
| | 26b and 25a, or | | | | |
| | (f) As (18a-e), except also use | | | | |
| | Design Solution 25b | | | | |
| (19) | Combine Table 53 Design Solutions | PSSR | C-HKR | PAFR | Spatial |
| | (a) As (18a-f), except delete Design | MLDR | | SCR | Print Tip |
| | Solutions 30b, c | PL-HKR | | LLSR | Print Plate |
| | | PS-HKR | | | Intensity |
| | | PSAR | | | Scale |
| (20) | Combine Table 53 Design Solutions | PSSR | — | PAFR | C-HKR |
| | (a) As (18a-f), except use Design | MLDR | | SCR | Spatial |
| | Solution 13a instead of Design | PL-HKR | | LLSR | Print Tip |
| | Solution 13b | PS-HKR | | | Print Plate |
| | | PSAR | | | Intensity |
| | | SCR | | | Scale |
| | | LLSR | | | |
| (21) | Combine Table 53 Design Solutions | PSSR | — | PAFR | C-HKR |
| | (a) As (19a), except use Design | MLDR | | SCR | Spatial |
| | Solution 13a instead of Design | PL-HKR | | LLSR | Print Tip |
| | Solution 13b | PS-HKR | | | Print Plate |
| | | PSAR | | | Intensity |
| | | | | | Scale |
| (22) | See note of Table 8 (17). | | | | |

TABLE 59
Preferred Practices for Design Solution Combinations Which Can Be Known to
Produce, Relative to the Prior Art, Improved Normalization for Pertinent UNFs
and/or CNFs, For All Microarray Measured Particular Gene RASR Values in an
Assay: Comparison of Directly Labeled SG Primed LPNs Produced From Isolated
mRNAs

| | | NFs | | Pertinent NFs | |
|---|---|---|---|---|---|
| | | Which Can Be | | To Be | |
| | | Ignored For | | Determined and | |
| | | Normalization | | Normalized For | |
| Combination of Assay Design Solutions | | UNF | CNF | UNF | CNF |
| | Compare Undegraded Isolated | PSSR | C-HKR | PAFR | Spatial |
| | mRNA Type 1 LPNs | MLDR | | SCR | Print Tip |
| (1) | Combine Table 53 Design Solutions | PL-HKR | | PSAR | Print Plate |
| | (a) 1, or 2, or 3, 4a, 5a, 6, 8a, c, 10b, | LLSR | | | Scale |
| | 13b, 14, 18a, 19, 21, 30a, b, 34, | SCR | | | Intensity |
| | or | PS-HKR | | | |
| | (b) 1, or 2, or 3, 4b, 5a, 6, 8a, c, 10b, | PSAR | | | |
| | 13b, 14, 18a, 19, 21, 30a, b, 34, or | | | | |
| | (c) As (1a-b), except use Design | | | | |
| | Solution 7 instead of Design | | | | |
| | Solution 6, or | | | | |
| | (d) As (1a-c), except delete Design | | | | |
| | Solutions 8a, c, or | | | | |
| | (e) As (1a-d), except use Design | | | | |
| | Solution 17b | | | | |
| (2) | Combine Table 53 Design Solutions | PSSR | C-HKR | PAFR | Spatial |
| | (a) As (1a-e), except delete Design | MLDR | | SCR | Print Tip |
| | Solution, 30a, b. | PL-HKR | | PSAR | Print Plate |
| | | PS-HKR | | | Intensity |
| | | LLSR | | | Scale |
| (3) | Combine Table 53 Design Solutions | PSSR | — | PAFR | C-HKR |
| | (a) As (1a-e), except use Design | MLDR | | SCR | Spatial |
| | Solution 13a instead of Design | PL-HKR | | PSAR | Print Tip |
| | Solution 13b | PS-HKR | | | Print Plate |
| | | LLSR | | | Intensity |
| | | SCR | | | Scale |
| | | PSAR | | | |
| (4) | Combine Table 53 Design Solutions | PSSR | — | PAFR | C-HKR |
| | (a) As (2a), except use Design | MLDR | | SCR | Spatial |
| | Solution 13a instead of Design | PL-HKR | | PSAR | Print Tip |
| | Solution 13b | PS-HKR | | | Print Plate |
| | | LLSR | | | Intensity |
| | | | | | Scale |
| | Compare Degraded Isolated mRNA | PSSR | C-HKR | PAFR | Spatial |
| | Type 1 LPNs | MLDR | | SCR | Print Tip |
| (5) | Combine Table 53 Design Solutions | PL-HKR | | PSAR | Print Plate |
| | (a) 1, or 2, or 3, 4a, 5a, 6, 8a, c, 10b, | PS-HKR | | | Intensity |
| | 13b, 14, 15a, 16a, 18a, 19, 30a, b, | LLSR | | | Scale |
| | 34 | SCR | | | |
| | or | PSAR | | | |
| | (b) 1, or 2, or 3, 4b, 5a, 6, 8a, c, 10b, | | | | |
| | 13b, 14, 15a, 16a, 18a, 19, 30a, b, | | | | |
| | 34 | | | | |
| | or | | | | |
| | (c) As (5a-b), except use Design | | | | |
| | Solution 15b instead of Design | | | | |
| | Solution 15a. | | | | |
| | or | | | | |
| | (d) As (5a-c), except use Design | | | | |
| | Solution 7 instead of Design | | | | |
| | Solution 6 | | | | |
| | or | | | | |
| | (e) As (5a-d), except delete Design | | | | |
| | Solutions 8a, c | | | | |
| | or | | | | |
| | (f) As (5a-e), except delete Design | | | | |
| | Solutions 1, 3, 16a, and 18a and | | | | |
| | use Design Solutions 16b, 18b, | | | | |
| | 17a, and 2 or 33 | | | | |
| | or | | | | |
| | (g) As (5a-f), except also use Design | | | | |
| | Solution 17b | | | | |
| (6) | Combine Table 53 Design Solutions | PSSR | C-HKR | PAFR | Spatial |
| | (a) As (5a-g), except delete Design | MLDR | | SCR | Print Tip |
| | Solutions 30a, b | PL-HKR | | PSAR | Print Plate |
| | | PS-HKR | | | Intensity |
| | | LLSR | | | Scale |
| (7) | Combine Table 53 Design Solutions | PSSR | — | PAFR | C-HKR |
| | (a) As (5a-g), except use Design | MLDR | | SCR | Spatial |
| | Solution 13a instead of Design | PL-HKR | | PSAR | Print Tip |
| | Solution 13b | PS-HKR | | | Print Plate |
| | | LLSR | | | Intensity |
| | | SCR | | | Scale |
| | | PSAR | | | |
| (8) | Combine Table 53 Design Solutions | PSSR | — | PAFR | C-HKR |
| | (a) As (6a), except use Design | MLDR | | SCR | Spatial |
| | Solution 13a instead | PL-HKR | | PSAR | Print Tip |
| | of Design Solution 13b | PS-HKR | | | Print Plate |
| | | LLSR | | | Intensity |
| | | | | | Scale |
| | Compare Undegraded Isolated | PSSR | C-HKR | PAFR | Spatial |
| | mRNA Type 2 LPNs | MLDR | | SCR | Print Tip |
| (9) | Combine Table 53 Design Solutions | PL-HKR | | LLSR | Print Plate |
| | (a) 1, or 2, or 3, 4a, 5b, 6, 8a, b, 10b, | PS-HKR | | | Intensity |
| | 13b, 14, 18a, 19, 21, 30b, c, 34 | PSAR | | | Scale |
| | or | SCR | | | |
| | (b) 1, or 2, or 3, 4b, 5b, 6, 8a, b, 10b, | LLSR | | | |
| | 13b, 14, 18a, 19, 21, 30a, c, 34 | | | | |
| | or | | | | |
| | (c) As (9a-b), except use Design | | | | |
| | Solution 7 instead of Design | | | | |
| | Solution 6 | | | | |
| | or | | | | |
| | (d) As (9a-c), except delete Design | | | | |
| | Solution 8a, b | | | | |
| | or | | | | |
| | (e) As (9a-d), except use Design | | | | |
| | Solution 17b | | | | |
| (10) | Combine Table 53 Design Solutions | PSSR | C-HKR | PAFR | Spatial |
| | (a) As (9a-e), except delete Design | MLDR | | SCR | Print Tip |
| | Solution 30b, c | PL-HKR | | LLSR | Print Plate |
| | | PS-HKR | | | Intensity |
| | | PSAR | | | Scale |
| (11) | Combine Table 53 Design Solutions | PSSR | — | PAFR | C-HKR |
| | (a) As (9a-e), except use Design | MLDR | | SCR | Spatial |
| | Solution 13a instead of Design | PL-HKR | | LLSR | Print Tip |
| | Solution 13b | PS-HKR | | | Print Plate |
| | | PSAR | | | Intensity |
| | | SCR | | | Scale |
| | | LLSR | | | |
| (12) | Combine Table 53 Design Solutions | PSSR | — | PAFR | C-HKR |
| | (a) As (10a), except use Design | MLDR | | SCR | Spatial |
| | Solution 13a instead of Design | PL-HKR | | LLSR | Print Tip |
| | Solution 13b | PS-HKR | | | Print Plate |
| | | PSAR | | | Intensity |
| | | | | | Scale |
| | Compare Degraded Isolated mRNA | PSSR | C-HKR | PAFR | Spatial |
| | Type 2 L-LPNs | MLDR | | SCR | Print Tip |
| (13) | Combine Table 53 Design Solutions | PL-HKR | | LLSR | Print Plate |
| | (a) 1, or 2, or 3, 4a, 5b, 6, 8a, b, 10b, | PS-HKR | | | Intensity |
| | 13b, 14, 15a, 16a, 18a, 19, 30b, c, | PSAR | | | Scale |
| | 34, or | SCR | | | |
| | (b) 1, or 2, or 3, 4b, 5b, 6, 8a, b, 10b, | LLSR | | | |
| | 13b, 14, 15a, 16a, 18a, 19, 30b, c, | | | | |
| | 34, or | | | | |
| | (c) As (13a-b), except use Design | | | | |
| | Solution 15b instead of Design | | | | |
| | Solution 15a, or | | | | |
| | (d) As (13a-c), except use Design | | | | |
| | Solution 7 instead of Design Solution | | | | |
| | 6, or | | | | |
| | (e) As (13a-d), except delete Design | | | | |
| | Solution 8a, b, or | | | | |
| | (f) As (13a-e), except delete Design | | | | |
| | Solutions 16a and 18a, and use | | | | |
| | Design Solutions 16b, 18b, and 17a, | | | | |
| | or | | | | |
| | (g) As (13a-f), except also use | | | | |
| | Design Solution 17b | | | | |
| (14) | Combine Table 53 Design Solutions | PSSR | C-HKR | PAFR | Spatial |
| | (a) As (13a-g), except delete | MLDR | | SCR | Print Tip |
| | Design Solutions 30, b, c | PL-HKR | | LLSR | Print Plate |
| | | PS-HKR | | | Intensity |
| | | PSAR | | | Scale |
| (15) | Combine Table 53 Design Solutions | PSSR | — | PAFR | C-HKR |
| | (a) As (13a-g), except use Design | MLDR | | SCR | Spatial |
| | Solution 13a instead of Design | PL-HKR | | LLSR | Print Tip |
| | Solution 13b | PS-HKR | | | Print Plate |
| | | PSAR | | | Intensity |
| | | SCR | | | Scale |
| | | LLSR | | | |
| (16) | Combine Table 53 Design Solutions | PSSR | — | PAFR | C-HKR |
| | (a) As (14a), except use Design | MLDR | | SCR | Spatial |
| | Solution 13a instead of Design | PL-HKR | | LLSR | Print Tip |
| | Solution 13b | PS-HKR | | | Print Plate |
| | | PSAR | | | Intensity |
| | | | | | Scale |
| (17) | See note of Table 8 (17). | | | | |

TABLE 60
Preferred Practices for Design Solution Combinations Which Can Be Known to Produce,
Relative to the Prior Art, Improved Normalization for Pertinent UNFs and/or CNFs,
For All or Essentially All Microarray Measured Particular Gene RASR Values in an
Assay: Comparison of Directly Labeled Random Primed LPNs Produced From
Isolated mRNAs

| | | NFs | | Pertinent NFs | |
|---|---|---|---|---|---|
| | | Which Can Be | | To Be | |
| | | Ignored For | | Determined and | |
| Combination of Assay Design | | Normalization | | Normalized For | |
| Solutions | | UNF | CNF | UNF | CNF |
| | Compare Type LPNs Produced From | PSSR | C-HKR | PAFR | Spatial |
| | Undegraded Isolated mRNA or | MLDR | | SCR | Print Tip |
| | Isolated mRNA Which Became | PL-HKR | | PSAR | Print Plate |
| | Degraded After Isolation | PS-HKR | | | Intensity |
| (1) | Combine Table 53 Design Solutions | LLSR | | | Scale |
| | (a) 1, or 2, or 3, 4a, 5a, 6, 8a, c, 12, | SCR | | | |
| | 13b, 14, 15b, 16a, 18a, 19, 30a, | PSAR | | | |
| | b, 34, or | | | | |
| | (b) 1, or 2, or 3, 4b, 5a, 6, 8a, c, 12, | | | | |
| | 13b, 14, 15b, 16a, 18a, 19, 30a, | | | | |
| | b, 34, or | | | | |
| | (c) As (1a-b), except use Design | | | | |
| | Solution 7 instead of Design | | | | |
| | Solution 6, or | | | | |
| | (d) As (1a-c), except delete Design | | | | |
| | Solution 8a, or | | | | |
| | (e) As (1a-d), except use Design | | | | |
| | Solutions 16a, and 18a and use | | | | |
| | Design Solutions 16b, 18b, and | | | | |
| | 17a, or | | | | |
| | (f) As (1a-e), except use Design | | | | |
| | Solution 17b | | | | |
| (2) | Combine Table 53 Design Solutions | PSSR | C-HKR | PAFR | Spatial |
| | (a) As (1a-e), except delete Design | MLDR | | SCR | Print Tip |
| | Solution, 30a, b. | PL-HKR | | PSAR | Print Plate |
| | | PS-HKR | | | Intensity |
| | | LLSR | | | Scale |
| (3) | Combine Table 53 Design Solutions | PSSR | — | PAFR | C-HKR |
| | (a) As (1a-e), except use Design | MLDR | | SCR | Spatial |
| | Solution 13a instead of Design | PL-HKR | | PSAR | Print Tip |
| | Solution 13b. | PS-HKR | | | Print Plate |
| | | LLSR | | | Intensity |
| | | SCR | | | Scale |
| | | PSAR | | | |
| (4) | Combine Table 53 Design Solutions | PSSR | — | — | C-HKR |
| | (a) As (3a), except use Design | MLDR | | | Spatial |
| | Solution 13a instead of Design | PL-HKR | | | Print Tip |
| | Solution 13b. | PS-HKR | | | Print Plate |
| | | PSAR | | | Intensity |
| | | | | | Scale |
| | Compare Type 1 LPNs Produced | PSSR | C-HKR | PAFR | Spatial |
| | From mRNAs Isolated From | MLDR | | SCR | Print Tip |
| | Degraded T-RNAs | PL-HKR | | PSAR | Print Plate |
| (5) | Combine Table 53 Design Solutions | PS-HKR | | | Intensity |
| | (a) 1, or 2, or 3, 4a, 5a, 6, 8a, c, 12, | LLSR | | | Scale |
| | 13b, 14, 15b, 16a, 18a, 19, 30a, | SCR | | | |
| | b, 34 | PSAR | | | |
| | or | | | | |
| | (b) 1, or 2, or 3, 4b, 5a, 6, 8a, c, 12, | | | | |
| | 13b, 14, 15b, 16a, 18a, 19, 30a, | | | | |
| | b, 34 | | | | |
| | or | | | | |
| | (c) As (1a-b), except use Design | | | | |
| | Solution 7 instead of Design | | | | |
| | Solution 6 | | | | |
| | or | | | | |
| | (d) As (1a-c), except delete Design | | | | |
| | Solutions 8a, c | | | | |
| | or | | | | |
| | (e) As (1a-d), except delete Design | | | | |
| | Solutions 1, 3, 16a, and 18a and | | | | |
| | use Design Solutions 16b, 18b, | | | | |
| | 17a, and 2 or 33 | | | | |
| | or | | | | |
| | (f) As (5a-e), except also use | | | | |
| | Design Solution 17b | | | | |
| (6) | Combine Table 53 Design Solutions | PSSR | C-HKR | PAFR | Spatial |
| | (a) As (5a-f), except delete Design | MLDR | | SCR | Print Tip |
| | Solutions 30a, b | PL-HKR | | PSAR | Print Plate |
| | | PS-HKR | | | Intensity |
| | | LLSR | | | Scale |
| (7) | Combine Table 53 Design Solutions | PSSR | — | PAFR | C-HKR |
| | (a) As (5a-f), except use Design | MLDR | | SCR | Spatial |
| | Solution 13a instead of Design | PL-HKR | | PSAR | Print Tip |
| | Solution 13b | PS-HKR | | | Print Plate |
| | | LLSR | | | Intensity |
| | | SCR | | | Scale |
| | | PSAR | | | |
| (8) | Combine Table 53 Design Solutions | PSSR | — | PAFR | C-HKR |
| | (a) As (7a), except use Design | MLDR | | SCR | Spatial |
| | Solution 13a instead of Design | PL-HKR | | PSAR | Print Tip |
| | Solution 13b | PS-HKR | | | Print Plate |
| | | LLSR | | | Intensity |
| | | | | | Scale |

Design solution combinations which can be known to provide improved normalization for all, or essentially all, particular gene comparison RASR values in an assay, are presented in Tables 61 through 67. While these design solution combinations provide improved normalization, they are not considered to be preferred methods because they rely on the determination of PL-HKR and PS-HKR UNF values for the assay, and the information necessary to determine these UNF values is currently unknown and must be obtained experimentally. Table 68 presents design solution combinations which can be known to more completely normalize only an identifiable subset of particular gene comparison RASR values for all pertinent UNFs and CNFs, while improved, but less complete, normalization occurs for all other particular gene comparison RASR values in the assay. Table 69 presents design solution combinations which can be known to minimize or eliminate the occurrence of UNF and CNF related false negative results and their associated RDMs. The design solution combinations presented in Tables 54 through 69 are only a few of a large number of different design solution combinations which can be known to provide improved normalization of microarray assay gene expression analysis and gene expression comparison assay results.

TABLE 61
Design Solution Combinations Which Can Be Known to Completely Normalize All or
Essentially All, Assay Particular Gene RASR Values for All Pertinent UNFs and
CNFs: Comparison of Cell Sample Directly Labeled mRNAs Produced from T-RNA

| | | NFs | | Pertinent NFs | |
|---|---|---|---|---|---|
| | | Which Can Be | | To Be | |
| | | Ignored For | | Determined and | |
| | | Normalization | | Normalized For | |
| Combination of Assay Design Solutions | | UNF | CNF | UNF | CNF |
| | Compare Label mRNA Type 1 LPNs | PSSR | C-HKR | SCR | Spatial |
| | From T-RNA | PAFR | | MLDR | Print Tip |
| (1) | Combine Table 53 Design Solutions | LLSR | | PL-HKR | Print Plate |
| | (a) 1, or 2, or 3, 4a, 5a, 6, 8a, 13b, 14, | SCR | | PS-HKR | Intensity |
| | 22a, 23b, 24b, 26b, 30 b, 31a-d | | | PSAR | Scale |
| | or | | | | |
| | (b) 1, or 2, or 3, 4b, 5a, 6, 8a, 13b, 14, | | | | |
| | 22a, 23b, 24b, 26b, 30 b, 31a-d | | | | |
| | or | | | | |
| | (c) As (1a-b), except use Design | | | | |
| | Solution 7 instead of Design | | | | |
| | Solution 6 | | | | |
| | or | | | | |
| | (d) As (1a-c), except delete Design | | | | |
| | Solution 8a | | | | |
| (2) | Combine Table 53 Design Solutions | PSSR | C-HKR | SCR | Spatial |
| | (a) As (1a-d), except delete Design | PAFR | | MLDR | Print Tip |
| | Solution, 30b | LLSR | | PL-HKR | Print Plate |
| | | | | PS-HKR | Intensity |
| | | | | PSAR | Scale |
| (3) | Combine Table 53 Design Solutions | PSSR | — | SCR | C-HKR |
| | (a) As (1a-d), except use Design | PAFR | | MLDR | Spatial |
| | Solution 13a instead of Design | LLSR | | PL-HKR | Print Tip |
| | Solution 13b | SCR | | PS-HKR | Print Plate |
| | | | | PSAR | Intensity |
| | | | | | Scale |
| (4) | Combine Table 53 Design Solutions | PSSR | — | SCR | C-HKR |
| | (a) As (2a), except use Design Solution | PAFR | | MLDR | Spatial |
| | 13a instead of Design Solution 13b | LLSR | | PL-HKR | Print Tip |
| | | | | PS-HKR | Print Plate |
| | | | | PSAR | Intensity |
| | | | | | Scale |
| | Compare Labeled mRNA | PSSR | C-HKR | PL-HKR | Spatial |
| | Type 2 LPN From T- | PAFR | | PS-HKR | Print Tip |
| | RNA | MLDR | | SCR | Print Plate |
| (5) | Combine Table 53 Design Solutions | PSAR | | LLSR | Intensity |
| | (a) 1, or 2, or 3, 4a, 5b, 6, 8a, 13b, 14, | SCR | | | Scale |
| | 22a, 23b, 24b, 26b, 30b, c, 31b, c, e | LLSR | | | |
| | or | | | | |
| | (b) 1, or 2, or 3, 4b, 5b, 6, 8a, 13b, 14, | | | | |
| | 22a, 23b, 24b, 26b, 30b, c, 31b, c, e | | | | |
| | or | | | | |
| | (c) As (1a-b), except use Design | | | | |
| | Solution 7 instead of Design | | | | |
| | Solution 6 | | | | |
| | or | | | | |
| | (d) As (1a-c), except delete Design | | | | |
| | Solutions 8a | | | | |
| (6) | Combine Table 53 Design Solutions | PSSR | C-HKR | PL-HKR | Spatial |
| | (a) As (1a-d), except delete Design | PAFR | | PS-HKR | Print Tip |
| | Solutions 30b, c | MLDR | | SCR | Print Plate |
| | | PSAR | | LLSR | Intensity |
| | | | | | Scale |
| (7) | Combine Table 53 Design Solutions | PSSR | — | PL-HKR | C-HKR |
| | (a) As (1a-d), except use Design | PAFR | | PS-HKR | Spatial |
| | Solution 13a instead of Design | MLDR | | SCR | Print Tip |
| | Solution 13b | PSAR | | LLSR | Print Plate |
| | | SCR | | | Intensity |
| | | LLSR | | | Scale |
| (8) | Combine Table 53 Design Solutions | PSSR | — | PL-HKR | C-HKR |
| | (a) As (2a), except use Design Solution | PAFR | | PS-HKR | Spatial |
| | 13a instead of Design Solution 13b | MLDR | | SCR | Print Tip |
| | | PSAR | | LLSR | Print Plate |
| | | | | | Intensity |
| | | | | | Scale |

TABLE 62
Design Solution Combinations Which Can Be Known to Completely Normalize All
or Essentially All, Assay Particular Gene RASR Values for All Pertinent UNFs and
CNFs: Comparison of Cell Sample Directly Labeled LPNs Produced from T-RNA By
SG Priming

| | | NFs | | Pertinent NFs | |
|---|---|---|---|---|---|
| | | Which Can Be | | To Be | |
| | | Ignored For | | Determined and | |
| Combination of Assay Design | | Normalization | | Normalized For | |
| Solutions | | UNF | CNF | UNF | CNF |
| | Comparison of Type 1 LPNs From | PSSR | C-HKR | SCR | Spatial |
| | Degraded or Undegraded T-RNA | PAFR | | MLDR | Print Tip |
| (1) | Combine Table 53 Design Solutions | SCR | | PL-HKR | Print Plate |
| | (a) 1, or 2, or 3, 4a, 5a, 6, 8a, 10a, 13b, | LLSR | | PS-HKR | Intensity |
| | 14, 15a, 16b, 18b, 30 b, 31a-d | | | PSAR | Scale |
| | or | | | | |
| | (b) 1, or 2, or 3, 4b, 5a, 6, 8a, 10a, 13b, | | | | |
| | 14, 15a, 16b, 18b, 30 b, 31a-d | | | | |
| | or | | | | |
| | (c) As (1a-b), except use Design | | | | |
| | Solution 15b instead of Design | | | | |
| | Solution 15a | | | | |
| | or | | | | |
| | (d) As (1a-c) except use Design | | | | |
| | Solution 7 instead of Design | | | | |
| | Solution 6 | | | | |
| | or | | | | |
| | (e) As (1a-d), except delete Design | | | | |
| | Solution 8a | | | | |
| (2) | Combine Table 53 Design Solutions | PSSR | C-HKR | SCR | Spatial |
| | (a) As (1a-e), except delete Design | PAFR | | MLDR | Print Tip |
| | Solution, 30b | LLSR | | PL-HKR | Print Plate |
| | | | | PS-HKR | Intensity |
| | | | | PSAR | Scale |
| (3) | Combine Table 53 Design Solutions | PSSR | — | SCR | C-HKR |
| | (a) As (1a-e), except use Design | PAFR | | MLDR | Spatial |
| | Solution 13a instead of Design | LLSR | | PL-HKR | Print Tip |
| | Solution 13b | SCR | | PS-HKR | Print Plate |
| | | | | PSAR | Intensity |
| | | | | | Scale |
| (4) | Combine Table 53 Design Solutions | PSSR | — | SCR | C-HKR |
| | (a) As (2a), except use Design Solution | PAFR | | MLDR | Spatial |
| | 13a instead of Design Solution 13b | SCR | | PL-HKR | Print Tip |
| | | | | PS-HKR | Print Plate |
| | | | | PSAR | Intensity |
| | | | | | Scale |
| | Comparison of Type 2 LPNs From | PSSR | C-HKR | SCR | Spatial |
| | T-RNAs | PAFR | | PL-HKR | Print Tip |
| (5) | Combine Table 53 Design Solutions | MLDR | | PS-HKR | Print Plate |
| | (a) 1, or 2, or 3, 4a, 5b, 6, 8a, 10a, 13b, | PSAR | | LLSR | Intensity |
| | 14, 15a, 16b, 18b, 30b, c, 31 b, c, e | SCR | | | Scale |
| | or | LLSR | | | |
| | (b) 1, or 2, or 3, 4b, 5b, 6, 8a, 10a, 13b, | | | | |
| | 14, 15a, 16b, 18b, 30b, c, 31 b, c, e | | | | |
| | or | | | | |
| | (c) As (1a-b), except use Design | | | | |
| | Solution 15b instead of 15a | | | | |
| | or | | | | |
| | (d) As (1a-c), except use Design | | | | |
| | Solution 7 instead of Design | | | | |
| | Solution 6 | | | | |
| | or | | | | |
| | (e) As (1a-d), except delete Design | | | | |
| | Solution 8a | | | | |
| (6) | Combine Table 53 Design Solutions | PSSR | C-HKR | SCR | Spatial |
| | (a) As (1a-e), except delete Design | PAFR | | PL-HKR | Print Tip |
| | Solutions 30b, c | MLDR | | PS-HKR | Print Plate |
| | | PSAR | | LLSR | Intensity |
| | | | | | Scale |
| (7) | Combine Table 53 Design Solutions | PSSR | — | SCR | C-HKR |
| | (a) As (1a-e), except use Design | PAFR | | PL-HKR | Spatial |
| | Solution 13a instead of Design | MLDR | | PS-HKR | Print Tip |
| | Solution 13b | PSAR | | LLSR | Print Plate |
| | | SCR | | | Intensity |
| | | LLSR | | | Scale |
| (8) | Combine Table 53 Design Solutions | PSSR | — | SCR | C-HKR |
| | (a) As (2a), except use Design Solution | PAFR | | PL-HKR | Spatial |
| | 13a instead of Design Solution 13b | MLDR | | PS-HKR | Print Tip |
| | | PSAR | | LLSR | Print Plate |
| | | | | | Intensity |
| | | | | | Scale |

TABLE 63
Design Solution Combinations Which Can Be Known to Completely Normalize All or
Essentially All, Microarray Assay Measured Particular Gene RASR Values for All
Pertinent UNFs and CNFs: Comparison of Cell Sample Directly Labeled LPNs
Produced By Random Priming of T-RNA

| | | NFs | | Pertinent NFs | |
|---|---|---|---|---|---|
| | | Which Can Be | | To Be | |
| | | Ignored For | | Determined and | |
| Combination of Assay Design | | Normalization | | Normalized For | |
| Solutions | | UNF | CNF | UNF | CNF |
| | Comparison of Type 1 LPN From | PSSR | C-HKR | SCR | Spatial |
| | T-RNA | PAFR | | MLDR | Print Tip |
| (1) | Combine Table 53 Design Solutions | LLSR | | PL-HKR | Print Plate |
| | (a) 1, or 2, or 3, 4a, 5a, 6, 8a, 11, 13b, | SCR | | PS-HKR | Intensity |
| | 14, 15b, 16b, 18b, 30b, 31a-d | | | PSAR | Scale |
| | or | | | | |
| | (b) 1, or 2, or 3, 4b, 5a, 6, 8a, 11, 13b, | | | | |
| | 14, 15b, 16b, 18b, 30b, 31a-d | | | | |
| | or | | | | |
| | (c) As (1a-b) except use Design | | | | |
| | Solution 7 instead of Design | | | | |
| | Solution 6 | | | | |
| | or | | | | |
| | (d) As (1a-c), except delete Design | | | | |
| | Solution 8a | | | | |
| (2) | Combine Table 53 Design Solutions | PSSR | C-HKR | SCR | Spatial |
| | (a) As (1a-d), except delete Design | PAFR | | MLDR | Print Tip |
| | Solution 30b | LLSR | | PL-HKR | Print Plate |
| | | | | PS-HKR | Intensity |
| | | | | PSAR | Scale |
| (3) | Combine Table 53 Design Solutions | PSSR | — | SCR | C-HKR |
| | (a) As (1a-d), except use Design | PAFR | | MLDR | Spatial |
| | Solution 13a instead of Design | LLSR | | PL-HKR | Print Tip |
| | Solution 13b | SCR | | PS-HKR | Print Plate |
| | | | | PSAR | Intensity |
| | | | | | Scale |
| (4) | Combine Table 53 Design Solutions | PSSR | — | SCR | C-HKR |
| | (a) As (2a), except use Design Solution | PAFR | | MLDR | Spatial |
| | 13a instead of Design Solution 13b | LLSR | | PL-HKR | Print Tip |
| | | | | PS-HKR | Print Plate |
| | | | | PSAR | Intensity |
| | | | | | Scale |

TABLE 64
Design Solution Combinations Which Can Be Known to Produce, Relative to the Prior
Art, Improved Normalization for Pertinent UNFs and/or CNFs, for All Microarray
Measured Particular Gene RASR Values in an Assay: Comparison of Oligo dT
Produced Directly Labeled LPN from T-RNA or Isolated mRNA

| | | NFs | | Pertinent NFs | |
|---|---|---|---|---|---|
| | | Which Can Be | | To Be | |
| | | Ignored For | | Determined and | |
| | | Normalization | | Normalized For | |
| Combination of Assay Design Solutions | | UNF | CNF | UNF | CNF |
| | Compare Type 1 LPNs | PSSR | C-HKR | PAFR | Spatial |
| (1) | Combine Table 53 Design Solutions | LLSR | | MLDR | Print Tip |
| | (a) 1, or 2, or 3, 4a, 5a, 6, 8a, 9a, b, 13b, | SCR | | PL-HKR | Print Plate |
| | 14, 15a, 16b, 18b, 30b, 31a-d | | | PS-HKR | Intensity |
| | or | | | PSAR | Scale |
| | (b) 1, or 2, or 3, 4b, 5a, 6a, 8a, 9a, b, | | | SCR | |
| | 13b, 14, 15a, 16b, 18b, 30b, 31a-d | | | | |
| | or | | | | |
| | (c) As (1a-b), except use Design | | | | |
| | Solution 15b instead of 15a | | | | |
| | or | | | | |
| | (d) As (1a-c), except use Design | | | | |
| | Solution 7 instead of Design | | | | |
| | Solution 6 | | | | |
| | Or | | | | |
| | (e) As (1a-d), except delete Design | | | | |
| | Solution 8a | | | | |
| (2) | Combine Table 53 Design Solutions | PSSR | C-HKR | PAFR | Spatial |
| | (a) As (1a-e), except delete Design | LLSR | | MLDR | Print Tip |
| | Solution 30b | | | PL-HKR | Print Plate |
| | | | | PS-HKR | Intensity |
| | | | | PSAR | Scale |
| | | | | SCR | |
| (3) | Combine Table 53 Design Solutions | PSSR | — | PAFR | C-HKR |
| | (a) As (1a-e), except use Design | LLSR | | MLDR | Spatial |
| | Solution 13a instead of Design | SCR | | PL-HKR | Print Tip |
| | Solution 13b | | | PS-HKR | Print Plate |
| | | | | PSAR | Intensity |
| | | | | SCR | Scale |
| (4) | Combine Table 53 Design Solutions | PSSR | — | PAFR | C-HKR |
| | (a) As (2a), except use Design Solution | LLSR | | MLDR | Spatial |
| | 13a instead of Design Solution 13b | | | PL-HKR | Print Tip |
| | | | | PS-HKR | Print Plate |
| | | | | PSAR | Intensity |
| | | | | SCR | Scale |
| | Compare Type 2 LPNs | PSSR | C-HKR | PAFR | Spatial |
| (5) | Combine Table 53 Design Solutions | MLDR | | PL-HKR | Print Tip |
| | (a) 1, or 2, or 3, 4a, 5b, 6, 8a, 9a, b, 13b, | PSAR | | PS-HKR | Print Plate |
| | 14, 15a, 16b, 18b, 30b, c, 31b, c, e | SCR | | SCR | Intensity |
| | or | LLSR | | LLSR | Scale |
| | (b) 1, or 2, or 3, 4b, 5b, 6, 8a, 9a, b, 13b, | | | | |
| | 14, 15a, 16b, 18b, 30b, c, 31b, c, e | | | | |
| | or | | | | |
| | (c) As (5a-b), except use Design | | | | |
| | Solution 15b instead of 15a | | | | |
| | or | | | | |
| | (d) As (5a-c), except use Design | | | | |
| | Solution 7 instead of Design | | | | |
| | Solution 6 | | | | |
| | or | | | | |
| | (e) As (5a-e), except delete Design | | | | |
| | Solution 8a | | | | |
| (6) | Combine Table 53 Design Solutions | PSSR | C-HKR | PAFR | Spatial |
| | (a) As (5a-e), except delete Design | MLDR | | PL-HKR | Print Tip |
| | Solutions 30b, c | PSAR | | PS-HKR | Print Plate |
| | | | | SCR | Intensity |
| | | | | LLSR | Scale |
| (7) | Combine Table 53 Design Solutions | PSSR | — | PAFR | C-HKR |
| | (a) As (5a-e), except use Design | MLDR | | PL-HKR | Spatial |
| | Solution 13a instead of Design | PSAR | | PS-HKR | Print Tip |
| | Solution 13b | SCR | | SCR | Print Plate |
| | | LLSR | | LLSR | Intensity |
| | | | | | Scale |
| (8) | Combine Table 53 Design Solutions | PSSR | — | PAFR | C-HKR |
| | (a) As (6a), except use Design Solution | MLDR | | PL-HKR | Spatial |
| | 13a instead of Design Solution 13b | PSAR | | PS-HKR | Print Tip |
| | | | | SCR | Print Plate |
| | | | | LLSR | Intensity |
| | | | | | Scale |

TABLE 65
Design Solution Combinations Which Can Be Known to Produce, Relative to the Prior
Art, Improved Normalization for Pertinent UNFs and/or CNFs, for All Microarray
Measured Particular Gene RASR Values in an Assay: Comparison of Directly
Labeled mRNA LPNs Produced From Isolated mRNA

| | | NFs | | Pertinent NFs | |
|---|---|---|---|---|---|
| | | Which Can Be | | To Be | |
| | | Ignored For | | Determined and | |
| Combination of Assay Design | | Normalization | | Normalized For | |
| Solutions | | UNF | CNF | UNF | CNF |
| | Compare Type 1 Labeled | PSSR | C-HKR | PAFR | Spatial |
| | Isolated mRNA LPNs | LLSR | | MLDR | Print Tip |
| (1) | Combine Table 53 Design Solutions | SCR | | PL-HKR | Print Plate |
| | (a) 1, or 2, or 3, 4a, 5a, 6, 8a, 13b, 14, | | | PS-HKR | Intensity |
| | 22b, 23b, 24b, 26b, 30b, 31a-d | | | SCR | Scale |
| | or | | | PSAR | |
| | (b) 1, or 2, or 3, 4b, 5a, 6, 8a, 13b, 14, | | | | |
| | 22b, 23b, 24b, 26b, 30b, 31a-d | | | | |
| | or | | | | |
| | (c) As (1a-b), except use Design | | | | |
| | Solution 7 instead of Design | | | | |
| | Solution 6 | | | | |
| | or | | | | |
| | (d) As (1a-c), except delete Design | | | | |
| | Solution 8a | | | | |
| (2) | Combine Table 53 Design Solutions | PSSR | C-HKR | PAFR | Spatial |
| | (a) As (1a-d), except delete Design | LLSR | | MLDR | Print Tip |
| | Solution 30b | | | PL-HKR | Print Plate |
| | | | | PS-HKR | Intensity |
| | | | | SCR | Scale |
| | | | | PSAR | |
| (3) | Combine Table 53 Design Solutions | PSSR | — | PAFR | C-HKR |
| | (a) As (1a-d), except use Design | LLSR | | MLDR | Spatial |
| | Solution 13a instead of Design | SCR | | PL-HKR | Print Tip |
| | Solution 13b | | | PS-HKR | Print Plate |
| | | | | SCR | Intensity |
| | | | | PSAR | Scale |
| (4) | Combine Table 53 Design Solutions | PSSR | — | PAFR | C-HKR |
| | (a) As (2a), except use Design Solution | LLSR | | MLDR | Spatial |
| | 13a instead of Design Solution 13b | | | PL-HKR | Print Tip |
| | | | | PS-HKR | Print Plate |
| | | | | PSAR | Intensity |
| | | | | SCR | Scale |
| | Compare Type 2 Labeled mRNA LPNs | PSSR | C-HKR | PAFR | Spatial |
| (5) | Combine Table 53 Design Solutions | MLDR | | PL-HKR | Print Tip |
| | (a) 1, or 2, or 3, 4a, 5b, 6, 8a, 13b, 14, | PSAR | | PS-HKR | Print Plate |
| | 22b, 23b, 24b, 26b, 30b, c, 31b, c, e | SCR | | SCR | Intensity |
| | or | LLSR | | LLSR | Scale |
| | (b) 1, or 2, or 3, 4b, 5b, 6, 8a, 13b, 14, | | | | |
| | 22b, 24b, 26b, 30b, c, 31b, c, e | | | | |
| | or | | | | |
| | (c) As (1a-b), except use Design | | | | |
| | Solution 7 instead of Design | | | | |
| | Solution 6 | | | | |
| | or | | | | |
| | (d) As (1a-c), except delete Design | | | | |
| | Solution 8a | | | | |
| (6) | Combine Table 53 Design Solutions | PSSR | C-HKR | PAFR | Spatial |
| | (a) As (1a-b), except delete Design | MLDR | | PL-HKR | Print Tip |
| | Solutions 30b, c | PSAR | | PS-HKR | Print Plate |
| | | | | SCR | Intensity |
| | | | | LLSR | Scale |
| (7) | Combine Table 53 Design Solutions | PSSR | — | PAFR | C-HKR |
| | (a) As (1a-d), except use Design | MLDR | | PL-HKR | Spatial |
| | Solution 13a instead of Design | PSAR | | PS-HKR | Print Tip |
| | Solution 13b | LLSR | | SCR | Print Plate |
| | | SCR | | LLSR | Intensity |
| | | | | | Scale |
| (8) | Combine Table 53 Design Solutions | PSSR | — | PAFR | C-HKR |
| | (a) As (6a), except use Design Solution | MLDR | | PL-HKR | Spatial |
| | 13a instead of Design Solution 13b | PSAR | | PS-HKR | Print Tip |
| | | | | SCR | Print Plate |
| | | | | LLSR | Intensity |
| | | | | | Scale |

TABLE 66
Design Solution Combinations Which Can Be Known to Produce, Relative to the Prior
Art, Improved Normalization for Pertinent UNFs and/or CNFs, for All Microarray
Measured Particular Gene RASR Values in an Assay: Comparison of SG Primed
Directly Labeled LPNs Produced From Isolated mRNA

| | | NFs | | Pertinent | |
|---|---|---|---|---|---|
| | | Which Can Be | | NFs To Be | |
| | | Ignored For | | Determined and | |
| | | Normalization | | Normalized For | |
| Combination of Assay Design Solutions | | UNF | CNF | UNF | CNF |
| | Compare Type 1 Labeled Isolated mRNA | PSSR | C-HKR | PAFR | Spatial |
| | LPNs | LLSR | | MLDR | Print Tip |
| (1) | Combine Table 53 Design Solutions | SCR | | PL-HKR | Print Plate |
| | (a) 1, or 2, or 3, 4a, 5a, 6, 8a, 10b, 13b, | | | PS-HKR | Intensity |
| | 14, 15a, 16b, 18b, 30b, 31a-d | | | PSAR | Scale |
| | or | | | SCR | |
| | (b) 1, or 2, or 3, 4b, 5a, 6, 8a, 10b, 13b, | | | | |
| | 14, 15a, 16b, 18b, 30b, 31a-d | | | | |
| | or | | | | |
| | (c) As (1a-b), except use Design | | | | |
| | Solution 15b instead of Design | | | | |
| | Solution 15a | | | | |
| | or | | | | |
| | (d) As (1a-c), except use Design | | | | |
| | Solution 7 instead of Design | | | | |
| | Solution 6 | | | | |
| | or | | | | |
| | (e) As (1a-d), except delete Design | | | | |
| | Solution 8a | | | | |
| (2) | Combine Table 53 Design Solutions | PSSR | C-HKR | PAFR | Spatial |
| | (a) As (1a-e), except delete Design | LLSR | | MLDR | Print Tip |
| | Solution 30b | | | PL-HKR | Print Plate |
| | | | | PS-HKR | Intensity |
| | | | | PSAR | Scale |
| | | | | SCR | |
| (3) | Combine Table 53 Design Solutions | PSSR | — | PAFR | C-HKR |
| | (a) As (1a-e), except use Design | LLSR | | MLDR | Spatial |
| | Solution 13a instead of Design | SCR | | PL-HKR | Print Tip |
| | Solution 13b | | | PS-HKR | Print Plate |
| | | | | PSAR | Intensity |
| | | | | SCR | Scale |
| (4) | Combine Table 53 Design Solutions | PSSR | — | PAFR | C-HKR |
| | (a) As (2a), except use Design Solution | LLSR | | MLDR | Spatial |
| | 13a instead of Design Solution 13b | | | PL-HKR | Print Tip |
| | | | | PS-HKR | Print Plate |
| | | | | PSAR | Intensity |
| | | | | SCR | Scale |
| | Compare Type 2 LPNs | PSSR | C-HKR | PAFR | Spatial |
| (5) | Combine Table 53 Design Solutions | MLDR | | PL-HKR | Print Tip |
| | (a) 1, or 2, or 3, 4a, 5b, 6, 8a, 10b, 13b, | PSAR | | PS-HKR | Print Plate |
| | 14, 15a, 16b, 18b, 30b, c, 31b, c, e | SCR | | SCR | Intensity |
| | or | LLSR | | LLSR | Scale |
| | (b) 1, or 2, or 3, 4a, 5b, 6, 8a, 10b, 13b, | | | | |
| | 14, 15a, 16b, 18b, 30b, c, 31b, c, e | | | | |
| | or | | | | |
| | (c) As (5a-b), except use Design | | | | |
| | Solution 15b instead of Design | | | | |
| | Solution 15a | | | | |
| | or | | | | |
| | (d) As (5a-c), except use Design | | | | |
| | Solution 7 instead of Design | | | | |
| | Solution 6 | | | | |
| | or | | | | |
| | (e) As (5a-d), except delete Design | | | | |
| | Solution 30b, c | | | | |
| (6) | Combine Table 53 Design Solutions | PSSR | C-HKR | PAFR | Spatial |
| | (a) As (5a-e), except delete Design | MLDR | | PL-HKR | Print Tip |
| | Solutions 30b, c | PSAR | | PS-HKR | Print Plate |
| | | | | SCR | Intensity |
| | | | | LLSR | Scale |
| (7) | Combine Table 53 Design Solutions | PSSR | — | PAFR | C-HKR |
| | (a) As (5a-e), except use Design | MLDR | | PL-HKR | Spatial |
| | Solution 13a instead of Design | PSAR | | PS-HKR | Print Tip |
| | Solution 13b | SCR | | SCR | Print Plate |
| | | LLSR | | LLSR | Intensity |
| | | | | | Scale |
| (8) | Combine Table 53 Design Solutions | PSSR | — | PAFR | C-HKR |
| | (a) As (6a), except use Design Solution | MLDR | | PL-HKR | Spatial |
| | 13a instead of Design Solution 13b | PSAR | | PS-HKR | Print Tip |
| | | | | SCR | Print Plate |
| | | | | LLSR | Intensity |
| | | | | | Scale |

TABLE 67
Design Solution Combinations Which Can Be Known to Produce, Relative to the Prior
Art, Improved Normalization for Pertinent UNFs and/or CNFs, for All Microarray
Measured Particular Gene RASR Values in an Assay: Comparison of Random
Primed Directly Labeled LPNs Produced From Isolated Cell Sample mRNA

| | | NFs Which | | Pertinent | |
|---|---|---|---|---|---|
| | | Can Be | | NFs To Be | |
| | | Ignored For | | Determined and | |
| | | Normalization | | Normalized For | |
| Combination of Assay Design Solutions | | UNF | CNF | UNF | CNF |
| | Comparison of Type 1 LPNs | PSSR | C-HKR | PAFR | Spatial |
| (1) | Combine Table 53 Design Solutions | LLSR | | MLDR | Print Tip |
| | (a) 1, or 2, or 3, 4a, 5a, 6, 8a, 12, 13b, | SCR | | PL-HKR | Print Plate |
| | 14, 15b, 16b, 18b, 30b, 31a-d | | | PS-HKR | Intensity |
| | or | | | PSAR | Scale |
| | (b) 1, or 2, or 3, 4b, 5a, 6, 8a, 12, 13b, | | | SCR | |
| | 14. 15b, 16b, 18b, 30b, 31a-d | | | | |
| | or | | | | |
| | (c) As (1a-b), except use Design Solution | | | | |
| | 7 instead of Design Solution 6 | | | | |
| | or | | | | |
| | (d) As (1a-c), except delete Design | | | | |
| | Solution 8a | | | | |
| (2) | Combine Table 53 Design Solutions | PSSR | C-HKR | PAFR | Spatial |
| | (a) As (1a-d), except delete Design | LLSR | | MLDR | Print Tip |
| | Solution 30b | | | PL-HKR | Print Plate |
| | | | | PS-HKR | Intensity |
| | | | | PSAR | Scale |
| | | | | SCR | |
| (3) | Combine Table 53 Design Solutions | PSSR | — | PAFR | C-HKR |
| | (a) As (1a-d), except use Design Solution | LLSR | | MLDR | Spatial |
| | 13a instead of Design Solution 13b | SCR | | PL-HKR | Print Tip |
| | | | | PS-HKR | Print Plate |
| | | | | PSAR | Intensity |
| | | | | SCR | Scale |
| (4) | Combine Table 53 Design Solutions | PSSR | — | PAFR | C-HKR |
| | (a) As (2a), except use Design Solution | LLSR | | MLDR | Spatial |
| | 13a instead of Design Solution 13b | | | PL-HKR | Print Tip |
| | | | | PS-HKR | Print Plate |
| | | | | PSAR | Intensity |
| | | | | SCR | Scale |

TABLE 68
Design Solution Combinations Which Can Be Known to Provide Improved
Normalization for All Particular gene Assay Comparisons, and More Complete
Normalization for An Identifiable Subset of Particular Genes

| | | NFs Which | | NFs Which Must Be | | |
|---|---|---|---|---|---|---|
| | | Can Be Ignored | | Determined For Particular | | |
| | | For Particular | | Gene Subset and | | |
| | | Gene Subset For | | Normalized For | | |
| | | More | | More | | |
| Combination of | | Completely | Rest of | Completely | | |
| Assay Design | | Normalized | Particular | Normalized | Rest of | Particular |
| Solutions | | Subset | Genes | Subset | Genes | |
| | Compare Type 1 LPNs | PSSR* | PSSR* | PAFR | PAFR | C-HKR |
| (1) | Combine Table 53 | MLDR* | LLSR | SCR | SCR | Spatial |
| | Design Solutions | PL-HKR* | | PSAR | PSAR | Print Tip |
| | (a) 1, or 2, or 3, 4a, 5a, 6, | PS-HKR* | | C-HKR | PL-HKR | Print Plate |
| | 9a, 13a, 14, 15b, 16b, | LLSR | | Spatial | PS-HKR | Intensity |
| | 20, 32 | | | Print Tip | MLDR | Scale |
| | | | | Print Plate | | |
| | | | | Intensity | | |
| | | | | Scale | | |
| (2) | Combine Table 53 | PSSR* | PSSR* | SCR | SCR | C-HKR |
| | Design Solutions | PAFR* | PAFR* | PAFR | PSAR | Spatial |
| | (a) As (2a), except use | MLDR* | LLSR | C-HKR | MLDR | Print Tip |
| | Design Solution 10a | PL-HKR* | | Spatial | PL-HKR | Print Plate |
| | instead of Design | PS-HKR* | | Print Tip | PS-HKR | Intensity |
| | Solution 9b | LLSR | | Print Plate | | Scale |
| | | | | Intensity | | |
| | | | | Scale | | |
| (3) | Combine Table 53 | PSSR* | PSSR* | SCR | SCR | C-HKR |
| | Design Solutions | PAFR* | PAFR* | LLSR | LLSR | Spatial |
| | (a) As (2a), except use | MLDR | PSAR | C-HKR | PL-HKR | Print Tip |
| | Design Solution 5a | PL-HKR* | MLDR | Spatial | PS-HKR | Print Plate |
| | instead of Design | PS-HKR* | | Print Tip | | Intensity |
| | Solution 5b | PSAR | | Print Plate | | Scale |
| | | | | Intensity | | |
| | | | | Scale | | |

*Assay value is equal to one.

TABLE 69
Design Solution Combinations Which Can Be Known to Minimize or Eliminate the
Occurrence of Microarray Assay Generated UNF and CNF Related False Negative
Results and Associated RDMs

| | |
|---|---|
| NFs | Pertinent |
| Which Can Be | NFs To Be |
| Ignored For | Determined and |
| Normalization | Normalized For |

| Combination of Assay Design Solutions | | UNF | CNF | UNF | CNF |
|---|---|---|---|---|---|
| (1) | Combine Table 53 Design Solutions | Any | Any | The Rest | The Rest |
| | (a) As described in Tables 54-68, | Pertinent | Pertinent | | |
| | except also use Design Solution 34 | UNF = 1 | CNF = 1 | | |

The known design solution combination associated with a microarray assay determines whether the assay can be known to be associated with improved normalization of assay measured particular gene RASR values, and the degree to which the normalization can be known to be improved, relative to prior art microarray normalization practice. As discussed, prior art microarray practice does not determine and normalize for pertinent UNFs, and in addition the key assumptions necessary for the valid prior art normalization of pertinent CNFs are known to be invalid for certain prior art microarray assays, and cannot be known to be valid for the large majority of, if not all, prior art microarray assays. Prior art microarray practice does not provide the information necessary for determining the design solution combination associated with a particular prior art microarray direct label LPN comparison assay. These factors create a situation where the design solution combination associated with any particular prior art microarray is not known. This means that, except for those prior art microarray assays which are known to be invalidly normalized for certain CNFs, and/or not normalized for certain UNFs, the completeness and validity of normalization for other prior art microarray assays results cannot be known. The prior art produced particular gene comparison NASR values for these assays are then, uninterpretable. It is possible, but not likely, that unknown to prior art microarray practice, a particular prior art microarray assay is associated with incomplete but improved normalization. Absent knowledge of the design solution combination associated with the prior art assay however, it cannot be known whether the assay is associated with improved normalization or not.

The design solution combination associated with a microarray assay determines the following. (i) the validity of the pertinent CNF normalization. (ii) the completeness of normalization for pertinent UNFs and CNFs. (iii) the fraction of particular gene comparison RASR values in the assay which can be maximally normalized for pertinent UNFs and CNFs.
(iv) the ease of determining the assay values for pertinent CNFs and UNFs. (v) ease and simplicity of the normalization process. (vi) biological accuracy of the normalized particular gene NASR values for an assay. (vii) the overall interpretability of the normalized particular gene comparison NASR values. (viii) the between and within assay intercomparability of the normalized particular gene comparison NASR values. (ix) the intercomparability of a microarray measured cell sample particular gene N-DGER value with a measured cell sample particular gene N-DGER value obtained with a different microarray or non-microarray assay method, for which the design solution combination associated with the assay is known. Here, if the microarray assay measured particular gene N-DGER value is biologically accurate then: the normalization is valid and complete; the particular gene N-DGER value can be validly interpreted as to quantitative extent of gene expression difference and direction of regulation change; the particular gene N-DGER value can be validly intercompared with other biologically accurate microarray or non-microarray particular gene N-DGER values which have been obtained with other microarray or non-microarray methods. It is desirable to maximize each of the above noted characteristics as much as is practical. Tables 55 and 56 present examples of preferred microarray design solution combinations which maximize many of these characteristics. It will be useful to discuss certain of these examples in more detail.

Table 55(13a) describes a preferred design solution combination with multiple optimum characteristics. Here design solution will be termed DS. This design solution combination can employ cDNA microarrays, or either type of oligonucleotide array (DS 1 or 2 or 3). Radioactivity (DS 4a) is used as a label since radioactive LLSRs are more readily determined and generally more accurate than LLSR values for non-radioactive labels. Type 2 radioactive LPNs are compared (DS 5b) since the MLDR and PSAR can be ignored for normalization, and the global UNF LLSR is easier to determine, and probably more accurate than the non-global PSAR values. DS 6 is used to ensure the valid normalization of the pertinent non-global CNFs. DS 8a and b are used in order to simplify the normalization process. Here the SCR must be measured in order to know the SCR, while the LLSR can be known by assay design. DS 10a specifies the use of SG primers to produce compared LPNs from cell sample T-RNAs. The combination of SG primers and T-RNA ensures that the PAFR UNF is not pertinent to the assay, and therefore can be ignored during normalization. Implicit in the use of SG primers is that the microarray CDPs must be designed to detect the SG primed LPN for each particular gene comparison. SG primers suitable for producing type 2 LPNs are rarely if ever used in prior art microarray practice. DS 13b indicates that each compared LPN is labeled with a different radioactive label, and that only one hybridization solution which contains both compared LPNs is used in the assay. This makes it possible to ignore the global CNF C-HKR during normalization. DS 14 specifies that each compared LPN label density be low enough so that the PSSR UNF can be ignored during normalization. DS 15a allows for the easier, more accurate determination of the LLSR and SCR. DS 15a is not practiced by the prior art but can be accomplished using a controlled chain termination method. DS 16a, 18a, and 19, make it easier to determine the LLSR and ensure that the PL-HKR, and PS-HKR UNFs can be ignored during normalization. Here MLDR is not pertinent and the PL-HKR and PL-HKR assay values equal one. DS 30b, c simplifies the normalization process, and in combination with SD 16a, 18a, 19, eliminates the occurrence in the assay of PL-HKR, PS-HKR, SCR, and LLSR related false negative results and associated RDMs. A major goal of this design solution combination is to eliminate the need to measure the assay values for PAFR, MLDR, PL-HKR, PS-HKR, PSSR, and PSAR. As discussed, it is not practical to measure the PAFR or PSSR assay values for all of the particular gene comparisons in a microarray assay, and all of the information necessary to determine the PL-HKR and PS-HKR values is not currently available. In addition the LLSR is more readily determined than the PSAR. The modification of this design solution to use non-radioactive labels, complicates the assay somewhat, but comparable results to the radioactive version can be obtained. Similarly, DS 15b can be used instead of DS 15a. As indicated in Table 55(13-16), there are a variety of permutations of design solution combinations which can be known to completely normalize all particular gene comparison RASR values in the assay, for all pertinent UNFs or CNFs.

Table 55(5b) describes another preferred design solution combination with multiple optimal characteristics. This design solution can employ cDNA microarrays or either version of the oligonucleotide arrays (DS 1 or 2 or 3). DS 4b specifies the use of non-radioactive label. Fluorescent labels are by far the most commonly used non-radioactive labels. DS 5a indicates the comparison of type 1 LPNs for the assay. The vast majority of prior art microarray assays compare type 1 LPNs. DS 6, 8a, 10a, 13a, 14, were discussed above. DS 15a allows for the easier, probably more accurate determination of PSAR and SCR assay values, and is not practiced by the prior art. The use of DS 15b instead of DS 15a, still allows the determination of the PSAR and SCR values. DS 16a, 18a, and 19 make it easier to determine the PSAR assay value and ensures that the MLDR, PL-HKR, PS-HKR UNFs do not have to be determined experimentally, and can be ignored during normalization. DS 30 a, b simplifies the normalization process and in combination with DS 16a, 18a, and 19 eliminates the occurrence in the assay of MLDR, PL-HKR, PS-HKR, PSAR and SCR related false negative results, and associated RDMs. Again a major goal of the assay design solution combination is to eliminate the need to measure the assay values for PSSR, PAFR, MLDR, PL-HKR and PS-HKR. As indicated in Table 55(5)-(8), there are a variety of permutations of design solution combinations which can be known to completely normalize all particular gene comparison RASR values in the assay for all pertinent UNFs and CNFs.

An example of one of these alternate preferred design solution combinations is Table 55(5f). Here DS 16b and 18b indicates that as synthesized, the nucleotide lengths of the compared type 1 LPNs are not the same. This commonly occurs in the prior art, and when such type 1 LPNs are compared in the microarray assay the assay values for the UNFs MLDR, PL-HKR, and PS-HKR cannot be known to equal one for each particular gene comparison in the assay, and must be determined and be normalized for. To avoid the necessity of determining and normalizing for the MLDR, PL-HKR, and PS-HKR UNFs, the design solution combination of DS 17a and DS2 or DS33 is used. DS 17a indicates that the nucleotide lengths of the compared LPNs are adjusted to have the same average nucleotide lengths, which is somewhat longer than the longest particular gene CDP on the microarray used for the assay. DS 2 or DS 33 specifies that the nucleotide lengths of the particular gene CDPs on the microarray, are preferably shorter than the nucleotide length of the shortest particular gene undegraded mRNA in the assay. This combination of DS 17a and DS 2 or DS 33 ensures that only one particular gene LPN molecule can hybridize to each CDP molecule, and that the compared LPN molecules which do hybridize are the same nucleotide length. Under these design solution conditions, the MLDR, PL-HKR, and PS-HKR assay values are equal to one for each particular gene comparison in the assay, and these UNFs can be ignored during the normalization process. As indicated in Table 55(5 a-f), many other design solutions can use this approach for ignoring these UNFs.

Prior art microarray practice often compares random primed cell sample LPNs. Table 56(1b) describes a preferred design solution combination which compares random primed LPNs and provides for the improved normalization of all particular gene comparisons in an assay for all pertinent UNFs and CNFs. This design solution combination is almost identical to the earlier discussed able 55(5b) which compared SG primed LPNs, and the role of the individual design solutions was discussed there. As indicated in Table 56(1-4), there are a variety of other design solution combinations involving random primed LPNs which can be known to normalize all particular gene comparisons in an assay for all pertinent UNFs and CNFs.

A large majority of prior art microarray assays involve the comparison of oligo dT primed cell sample LPNs. As discussed, for the comparison of such oligo dT primed LPNs the non-global UNF PAFR is pertinent to the assay, and for any particular gene comparison in the assay the PAFR value may or may not equal one. Further, it is not practical to experimentally determine the PAFR for more than a few particular gene comparisons in the assay. In effect therefore, when oligo dT primed LPNs are compared in a microarray assay, the PAFR values for the particular gene comparison in the assay cannot be known. Prior art microarray practice has tacitly assumed that all, or the vast majority of, eukaryotic particular gene mRNAs in a cell sample are significantly polyadenylated, and therefore capable of being isolated by oligo dT affinity binding. In other words the prior art assumes that all, or virtually all, particular gene comparisons have in effect, a PAFR value equal to one. In this context, Table 57(5b) presents a preferred design solution combination involving the comparison of oligo dT primed LPNs, which can be known to produce improved normalization of all particular gene comparison RASR values for all pertinent UNFs and CNFs, except PAFR. This design solution combination is very similar to those of Table 55 (5b), and Table 56(1b). The role of the individual design solutions was discussed in these earlier examples. As indicated in Table 57(1-16), there are a variety of other design solution combinations which provides improved normalization of all particular gene comparison RASR values in the assay for all pertinent UNFs and CNFs, except PAFR.

The preferred and other design solution combinations described in Tables 54 through 69 represent only a fraction of the possible design solution combinations which can provide improved normalization. As an example, Table 68 presents design solution combinations which provide different degrees of improved normalization for different identifiable subsets of particular gene comparison RASR values in the same assay. One identifiable subset of particular gene comparison RASR values is normalized for only certain pertinent UNFs, and all pertinent CNFs. A different identifiable subset of particular gene comparison RASR values in the same assay, is normalized for all, or all but one, pertinent UNF and all pertinent CNFs. For the design solution combination presented in Table 68(2a), one identifiable subset of particular gene comparisons can be known to be normalized for all pertinent UNFs and CNFs, while a different identifiable subset of particular gene comparisons can be known to be normalized for all pertinent CNFs and only certain UNFs. Here, DS 4a specifies the comparison of radioactive LPNs, but non-radioactive LPNs can also be used. DS 6 specifies the use of standards to accomplish the known valid normalization of pertinent CNFs. DS 10a and 14 indicate the use of SG primers to produce type 1 LPNs from cell sample T-RNA, and the PSSR is not pertinent for the assay. DS 15b and 16b indicate that within a cell sample LPN prep the nucleotide lengths of different particular gene LPNs are not the same, and that the average nucleotide length of the compared LPN preps is not the same. This situation occurs often in the prior art. DS 20 indicates that a subset of the particular gene comparisons in the assay involve the comparison of particular gene LPNs which have the same nucleotide lengths, and that a subset of particular gene comparisons in the same assay do not compare particular gene LPNs of the same nucleotide lengths. DS 32 indicates that the average nucleotide length of each compared cell sample LPN prep is greater than the nucleotide lengths of undegraded mRNA molecules for one or more, but not all, different particular genes in the assay. DS 20 and 32 can be illustrated by considering hypothetical, but realistic SG primed mammalian cell sample LPN preps, which have the following characteristics. (a) there is only one SG priming site for each particular gene mRNA in the assay, and the priming site is located at the very extreme 3′ end of each different particular gene mRNA molecule. (b) undegraded mRNAs for different particular genes range in nucleotide length from about 200 nucleotides to around 7000 nucleotides or more, and the average undegraded mRNA nucleotide length for most mammalian cell sample mRNA preps is around 2000 nucleotides. (c) the average nucleotide length of cell sample one SG primed LPN prep is around 1600 nucleotides, while the average nucleotide length of cell sample two SG primed LPN prep is 800 nucleotides. (d) the average nucleotide length of each compared LPN prep is long enough so that the particular gene LPN molecule populations in both compared LPN preps which represent short, 300 to 500 or so, nucleotide long undegraded mRNAs, will consist entirely, or almost entirely, of LPN molecules which have the same nucleotide length as the undegraded particular gene mRNAs which produced them. As a result, in the microarray assay the subset of particular gene comparisons which represents these short particular gene mRNAs, can be known to involve the comparison of LPN molecules of the same nucleotide length. Further, for these particular short gene comparisons it can be known that the assay values for the pertinent UNFs MLDR, PL-HKR, and PS-HKR, are equal to one, and can therefore be ignored in the normalization process. (e) in this same assay, the compared LPN molecules which represent longer particular gene mRNAs will not have the same nucleotide lengths as the particular gene undegraded mRNAs which they represent. Further, the nucleotide lengths of the compared longer particular gene LPNs will not be the same in the assay. As a result, in the microarray assay the subset of particular gene comparisons which represents these longer particular gene mRNAs can be known to involve the comparison of particular gene LPNs which have different nucleotide lengths. Thus, it can be known for this subset of gene comparisons in the assay that the assay values for the pertinent UNFs MLDR, PL-HKR, and PS-HKR cannot be known to equal one. Further as discussed, when the compared particular gene LPNs are not the same in nucleotide length and nucleotide sequence, it is not currently possible to determine the assay values for the particular gene PL-HKR and PS-HKR UNFs. As a result of (a)-(e), while the table 68(2a) design solution combination provides improved normalization for all particular gene comparisons in the assay, some particular gene comparisons are normalized more completely than others. Table 68(1-3) presents other versions of this same basic normalization pattern, and many others exist which are not described here.

Tables 61 through 67 present microarray assay design solution combinations which provide improved normalization for all particular gene comparisons in an assay, but not for all pertinent UNFs and CNFs. Common to all of the Table 61 through 67 design solution combinations, is the comparison of particular gene LPNs which do not have the same nucleotide length. Most if, not all, of these design solution combinations describe microarray assays which, like those of Table 68, provide improved normalization for all particular gene comparisons in an assay, but provide more complete normalization for some particular gene comparisons than others. Again, the cause of this is the inability to determine the assay PL-HKR and PS-HKR values for each particular gene comparison in the assay. It is likely that the majority of prior art microarray assays have a similar, albeit unknown to the prior art, problem. These Table 61 through 67 design solution combinations will become more preferred when the basis for determining PL-HKR and PS-HKR assay values for particular gene comparisons of different nucleotide length LPNs, is established.

Table 69 presents microarray assay design solution combinations which can be known to minimize the occurrence of UNF and CNF related false negative results and their associated RDMs. As indicated in DS 34, this can be accomplished by maximizing the number of pertinent UNF and CNF assay values which equal one, or nearly one. As indicated in Tables 54 through 60, DS 34 can be incorporated into a large number of different design solution combinations. Although DS 34 has not been specified for the design solution combinations of Tables 61 through 69, it could be.

Note that the earlier discussed normalization of the prior art microarray assay measured slow vs fast growing bacteria RNA comparison assay N-DGER results with the SCR UNF, represents an example of improved normalization for a directly labeled LPN assay comparison.

A microarray assay can be described by the design solution combination which is associated with the assay. An accurate assay design solution combinations description serves as the basis for identifying the following. (i) the pertinent UNFs and CNFs which are associated with the assay. (ii) the pertinent UNFs and CNFs which can be ignored during the assay normalization process. (iii) the pertinent UNFs and CNFs assay values which must be determined and normalized for in the assay. (iv) the pertinent UNFs and CNFs which can be determined and normalized for. (v) the pertinent UNFs and CNFs which are normalized for. (vi) the assumptions necessary to determine UNF and CNF assay values. Such an overall description is necessary in order to evaluate the utility, biological accuracy, and intercomparability, of the assay measured particular gene comparison NASR values. Such an overall description should be available for every microarray assay. Such an overall design solution combination description can be used to plan future microarray assays, and to interpret already existing microarray assay particular gene comparison normalized results or NASR values. Such overall design solution combination descriptions were not created for prior art microarray assays of any kind. In addition such an overall design solution combination description will allow the effective standardization of microarray assay formats.

Improvement of the Prior Art Microarray Assay Normalization Process for Indirect Label L-LPN Assays by Assay Design, and Measurement of UNF and CNF Assay Values.

A large number of assay variables are associated with prior art microarray gene comparison indirect label L-LPN assays. Herein these assays will be termed L-LPN assays. As many as 13 different NFs may be pertinent for a type 1 L-LPN comparison assay. For a type 2 L-LPN assay as many as 11 different NFs may be pertinent for an assay. Only a small fraction of prior art indirect label assays involve the comparison of type 2 L-LPNs.

In order to accurately and completely normalize particular gene RASR values produced by such indirectly labeled type 1 or type 2 L-LPN assays, it is necessary to determine, or know, an accurate quantitative value for each NF which is pertinent for the assay measured particular gene RASR value, and then to normalize the particular gene RASR value for the pertinent NF values. The determination of such pertinent NF quantitative values and their use for normalization was discussed earlier. While the determination of global assay variables is generally practical the determination can still be complex, as for example, the determination of the assay SCR value. In contrast, determination of the assay values for particular gene non-global NFs can be quite complex, and has been described earlier. The non-global CNFs can be determined and normalized for in a straightforward manner using standards, as well as with well established prior art methods which are currently used, if it can be established that prior art normalization assumptions are valid. The determination of the assay values for particular gene non-global UNFs can be much more complex. For type 1 L-LPN assay comparisons, the determination of particular gene assay values for the non-global UNF MLDR can be done by a combination of inference and measurement as described earlier. Determination of the PL-HKR and PS-HKR UNF assay values is complex and requires information not currently known, but which can be obtained. Absent such information, the PL-HKR and PS-HKR values cannot be directly measured for many assay situations. A similar situation exists for the determination of the SBNR non-global variable UNF. In addition, it is impractical to determine the PAFR for each particular gene comparison in an assay, even for low density arrays.

It is useful to describe the pertinent NFs which are associated with prior art microarray indirectly labeled cell sample type 1 L-LPN prep comparison assays. The large majority of prior art indirect label L-LPN comparisons involve oligo dT or random primed L-LPNs. A small fraction of prior art L-LPN comparisons involve specific gene primed L-LPNs. The pertinent UNFs and CNFs associated with these type 1 L-LPN assays are presented in Tables 70 and 71. Tables 71 and 72 present the UNFs and CNFs which may be pertinent for a prior art type 2 L-LPN comparison assay. Note that the UNFs MLDR and SSAR, are not pertinent for a type 2 L-LPN assay comparison, but the LLSR and SBNR are.

Each of the prior art microarray assay situations described in Tables 70 through 72 represents a prior art microarray general assay situation, and the CNFs and UNFs which must be determined, or known, and normalized for in order to obtain improved microarray measured particular gene NASR and N-DGER values, and biologically accurate particular gene NASR values. In order to obtain such improved particular gene NASR values for prior art microarray L-LPN assays, the following improvements in the prior art normalization process are required. (i) it is necessary to use an improved normalization approach which can be known to be valid, or to know that the key prior art normalization assumptions are valid, in order to determine the pertinent CNF values and normalize for them. (ii) it is necessary to use an improved overall process for the more complete and accurate normalization of microarray L-LPN assay measured particular gene RASR values, which includes the identification of pertinent UNFs and CNFs for the assay, the valid and accurate determination of the pertinent UNF and CNF assay values, and the valid and accurate normalization for the pertinent UNF and CNF values.

TABLE 70
UNFs Associated with Prior Art Microarray Assay Comparisons of
Type 1 Indirect Label L-LPNs

| | Pertinent UNFs | | | |
|---|---|---|---|---|
| | When Comparing | | Pertinent UNFs | |
| | Isolated Cell | | When Comparing | |
| | Sample mRNAs | | Cell Sample T-RNAs | |
| | One Label | Two Label | One Label | Two Label |
| Primer Used | Assay | Assay | Assay | Assay |
| Oligo dT | SCR | SCR | SCR | SCR |
| | PAFR | PAFR | PAFR | PAFR |
| | MLDR | MLDR | MLDR | MLDR |
| | PL-HKR | PL-HKR | PL-HKR | PL-HKR |
| | PS-HKR | PS-HKR | PS-HKR | PS-HKR |
| | SBNR | SBNR | SBNR | SBNR |
| | SSAR | SSAR | SSAR | SSAR |
| Random | SCR | SCR | SCR | SCR |
| or | PAFR | PAFR | — | — |
| SG Primer | MLDR | MLDR | MLDR | MLDR |
| Mixture | PL-HKR | PL-HKR | PL-HKR | PL-HKR |
| | PS-HKR | PS-HKR | PS-HKR | PS-HKR |
| | SBNR | SBNR | SBNR | SBNR |
| | SSAR | SSAR | SSAR | SSAR |

TABLE 71
CNFs Associated with Prior Art Microarray Assay Comparisons of
Type 1 Indirect Label L-LPNs and Type 2 L-LPN Comparisons

| | Pertinent CNFs | | | |
|---|---|---|---|---|
| | When Comparing | | Pertinent CNFs | |
| | Isolated Cell | | When Comparing | |
| | Sample mRNAs | | Cell Sample T-RNAs | |
| | One Label | Two Label | One Label | Two Label |
| Primer Used | Assay | Assay | Assay | Assay |
| Oligo dT | C-HKR | — | C-HKR | — |
| or | Spatial | Spatial | Spatial | Spatial |
| Random | Print Tip | Print Tip | Print Tip | Print Tip |
| or | Print Plate | Print Plate | Print Plate | Print Plate |
| SG Primer | Intensity | Intensity | Intensity | Intensity |
| Mixture | Scale | Scale | Scale | Scale |

TABLE 72
UNFs Associated with Prior Art Microarray Assay Comparisons of
Type 2 Indirect Label L-LPNs

| | Pertinent UNFs | | Pertinent UNFs | |
|---|---|---|---|---|
| | When Comparing | | When | |
| | Isolated Cell | | Comparing Cell | |
| | Sample mRNAs | | Sample T-RNAs | |
| | One Label | Two Label | One Label | Two Label |
| Primer Used | Assay | Assay | Assay | Assay |
| Oligo dT | SCR | SCR | SCR | SCR |
| | PAFR | PAFR | PAFR | PAFR |
| | PL-HKR | PL-HKR | PL-HKR | PL-HKR |
| | PS-HKR | PS-HKR | PS-HKR | PS-HKR |
| | LLSR | LLSR | LLSR | LLSR |
| | SBNR | SBNR | SBNR | SBNR |
| SG Primer | SCR | SCR | SCR | SCR |
| Mixture | PAFR | PAFR | — | — |
| | PL-HKR | PL-HKR | PL-HKR | PL-HKR |
| | PS-HKR | PS-HKR | PS-HKR | PS-HKR |
| | LLSR | LLSR | LLSR | LLSR |
| | SBNR | SBNR | SBNR | SBNR |

Prior art microarray L-LPN practice does not determine the assay value for, or normalize particular gene RASR values for global or non-global UNFs. The majority of these prior art L-LPN assays involve the comparison of cell sample oligo dT primed and/or random primed type 1 L-LPN preps. For such assays as many as thirteen NFs may be pertinent to the assay, and seven of these are UNFs. Each UNF can cause an assay measured particular gene RASR value to deviate significantly from biological accuracy when the UNF value deviates significantly from one. These UNFs have practical meaning for the assay only if their individual deviations from one, or the product of their individual deviations from one, are significantly large relative to the measurement accuracy of the assay. Table 73 presents what are considered to be conservative estimates for the deviations from one which are believed to occur commonly for prior art L-LPN assays. The commonly claimed prior art microarray assay measurement accuracy is also presented. In the context of the measurement accuracy of a typical prior art microarray assay, the deviation of even one of these UNFs is large enough to significantly affect the quantitative value and interpretation of a prior art measured particular gene N-DGER or NASR value. Therefore such deviations from one have significant practical importance for the interpretation of prior art produced N-DGER or NASR values, and for the future production of biologically accurate microarray measured N-DGER or NASR values.

TABLE 73
Estimated Magnitude of Deviation of NFs from One and Biological Accuracy, for a
Microarray Assay Indirect Label L-LPN Comparisons

| | | Estimated Deviation of NF Value From | | |
|---|---|---|---|---|
| | | One For A Typical Prior Art Microarray | | |
| | | Assay | | |
| | | Conservative | Measurement | |
| NF Type | | Commonly | Plausible Potential | Accuracy of Prior Art |
| UNF | CNF | Occurring Deviation | Deviation | Microarray Assays |
| SCR | | 6 Fold | 20-25 Fold | The measurement of |
| PAFR | | 1.33 Fold | 3 Fold | accurate N-DGER |
| MLDR | | 3 Fold | 10-20 Fold | values to within ±1.2 |
| PL-HKR | | 1.5 Fold | 3 Fold | fold to 4 fold is often |
| PS-HKR | | 1.5 Fold | >2 Fold | claimed. |
| LLSR | | 1.5 Fold | >5 Fold | Generally, the claim is |
| SBNR | | 2 Fold | >4 Fold | ±1.5 to 2 fold. |
| SSAR | | 1.5 Fold | >3 Fold | |
| | C-HKR | 2 Fold | >3 Fold | |
| | Spatial | 2 Fold | >3 Fold | |
| | Print Tip | 2 Fold | >3 Fold | |
| | Print Plate | 2 Fold | >3 Fold | |
| | Intensity | 2 Fold | >3 Fold | |
| | Scale | 2 Fold | >3 Fold | |

Further, because prior microarray practice does not determine the UNF assay values, it cannot be known whether a prior art measured particular gene NASR or N-DGER value requires normalization for the pertinent UNFs or not. Therefore, it is necessary to first identify the UNFs which are pertinent for an assay, and then to determine a quantitative measure of each pertinent UNFs assay value, in order to determine whether normalization is necessary for the UNF, and then to normalize the assay measured particular gene RASR value for the UNF. For a typical microarray L-LPN assay the requirement to determine and normalize for the assay pertinent UNF values adds a very significant amount of complexity and effort to the assay, relative to the prior art microarray practice. In addition, a significant amount of systematic measurement error and noise may be associated with the experimentally determined UNF values, and their use for normalization. Further, the use of the improve method for determining and normalizing for the assay pertinent CNFs, also adds additional complexity and effort to the microarray assay, relative to prior art practice. These considerations make it very desirable, if not necessary, to simplify the determination of L-LPN assay pertinent CNFs, and the normalization process, as much as possible, and to eliminate the necessity for determining as many UNFs and CNFs as possible.

Earlier sections extensively discussed the underlying basis for each UNF, and the assay situations under which each UNF or CNF is pertinent. As a result it is possible to identify assay factors which can and must be controlled for different assay situations or formats in order to simplify the process of determining the pertinent UNF and CNF values, and normalizing for them. This knowledge makes it possible to knowingly design microarray L-LPN assays which do not require the direct determination of certain UNFs and CNFs in order to validly normalize for these NFs. Further the knowledge makes it possible to reduce the number of pertinent UNFs and CNFs which are associated with a microarray assay. The overall result of such designs is a simplified version of the improved microarray L-LPN assay normalization process. This can be accomplished by judicious assay design and measurement, as is discussed and described below.

The various general design approaches which will provide an improved normalization process relative to the prior art normalization processes, are presented in Table 52. The successful implementation of any one of the Table 52 design approaches 1-8, will produce a normalization process which can be known to be improved, relative to prior art normalization practices. The successful implementation of Table 52 design approach 9, will produce microarray assay results which are known to contain fewer NF related false negative results than occur for prior art results.

Prior art microarray L-LPN assay design is not standardized, and there are a variety of different microarray assay formats practiced by the prior art. These have been extensively discussed earlier. The improvement of the normalization process for these microarray L-LPN formats will be discussed. The design solutions or design components which can be used to produce improved microarray L-LPN normalization are presented in Table 74. Each of these design solutions or components reflects an aspect of microarray L-LPN assay design which either directly or indirectly impacts an assay pertinent NF, and/or the simplification of the normalization process. Different combinations of these design solutions can be used to describe an overall microarray L-LPN assay.

TABLE 74
Design Solutions for Further Improving the Microarray Assay Normalization Process
and the Assay Measured Particular Gene NASR Values Obtained Using Indirectly
Labeled L-LPNs

| | | NFs | | Reason For | |
|---|---|---|---|---|---|
| | | Which Can Be | | Ignoring NFs | |
| | | Ignored During | | (NP = Not | |
| | | Normalization | | Pertinent) | |
| Assay Design Solutions | | UNF | CNF | UNF | CNF |
| (1) | Use cDNA microarray. | — | — | — | — |
| (2) | Use an oligonucleotide microarray | — | — | — | — |
| | which contains only one CDP | | | | |
| | sequence specific for each different | | | | |
| | gene mRNA to be detected. | | | | |
| (3) | Use an oligonucleotide microarray | — | — | — | — |
| | which contains multiple CDP | | | | |
| | sequences specific for each different | | | | |
| | gene mRNA to be detected. | | | | |
| (4) | Use (a) Radioactive Label | — | — | — | — |
| | (b) Non-radioactive label | | | | |
| | As the signal generating entity | | | | |
| (5) | Compare (a) Type 1 L-LPNs | LLSR | — | NP | — |
| | (b) Type 2 L-LPNs | MLDR | | NP | |
| | | SBNR | | NP | |
| | | SSAR | | NP | |
| (6) | Use standards to validly normalize for | — | — | — | — |
| | pertinent global and non-global CNFs. | | | | |
| (7) | Use prior art method to normalize for | — | — | — | — |
| | pertinent global and non-global CNFs, | | | | |
| | after establishing the validity of the | | | | |
| | prior art normalization method for the | | | | |
| | assay. | | | | |
| (8) | Use AHG and/or other standards to | — | — | — | — |
| | determine and normalize for | | | | |
| | (a) SCR | | | | |
| | (b) LLSR | | | | |
| | (c) PSAR | | | | |
| | (d) SSAR | | | | |
| (9) | Compare oligo dT primed LPNs | — | — | — | — |
| | produced from | | | | |
| | (a) Cell Sample T-RNA | | | | |
| | (b) Cell Sample Isolated mRNA | | | | |
| (10) | Compare SG primed L-LPNs produced | PAFR | — | NP | — |
| | from | — | — | — | — |
| | (a) Cell sample T-RNAs | | | | |
| | (b) Cell sample isolated mRNAs | | | | |
| (11) | Compare random primed LPNs made | PAFR | — | NP | — |
| | from cell sample T-RNAs. | | | | |
| (12) | Compare random primed LPNs made | — | — | — | — |
| | from cell sample isolated mRNAs. | | | | |
| (13) | Use (a) One ligand for assay | — | — | — | — |
| | (b) Two ligands for assay | — | C-HKR | — | C-HKR = 1 |
| (14) | Use low enough signal molecule | — | — | — | — |
| | density to avoid signal label molecule | | | | |
| | density effects | | | | |
| (15) | The synthesized L-LPN nucleotide | — | — | — | — |
| | lengths for the L-LPN molecules in a | | | | |
| | cell sample L-LPN prep are | | | | |
| | (a) The same | | | | |
| | (b) Different | | | | |
| (16) | The average synthesized L-LPN | MLDR* | — | =1 | — |
| | nucleotide lengths of compared cell | PL-HKR* | | — | |
| | sample L-LPN preps are | PS-HKR* | | | |
| | (a) The same | — | | | |
| | (b) Different | | | | |
| (17) | Compared cell sample L-LPN preps | MLDR* | — | =1 | — |
| | are synthesized and then adjusted to | PL-HKR* | | =1 | |
| | have nucleotide lengths which are | PS-HKR* | | | |
| | somewhat longer than the longest CDP | | | | |
| | on the microarray, and which have | | | | |
| | (a) The same average L-LPN | | | | |
| | nucleotide lengths | | | | |
| | (b) As (a) except that the average L- | | | | |
| | LPN nucleotide lengths are much | | | | |
| | smaller than in (a) | | | | |
| (18) | Synthesized L-LPN nucleotide lengths | MLDR* | — | =1 | — |
| | for the compared particular gene L- | PL-HKR* | | | |
| | LPNs are | PS-HKR* | | | |
| | (a) The same | | | | |
| | (b) Different | | | | |
| (19) | Synthesized L-LPN nucleotide lengths | MLDR | — | =1 | — |
| | and nucleotide sequences are the same | PL-HKR | | | |
| | or essentially the same for all | PS-HKR | | | |
| | compared particular gene L-LPNs in | | | | |
| | the assay. | | | | |
| (20) | Synthesized L-LPN nucleotide length | MLDR | — | =1 | — |
| | and nucleotide sequences are the same | PL-HKR | | | |
| | or essentially the same for less than all | PS-HKR | | | |
| | compared particular gene L-LPNs in | | | | |
| | the assay. | | | | |
| (21) | Compare synthesized particular gene | MLDR | — | =1 | — |
| | L-LPNs which are equal in length to | PL-HKR | | | |
| | each particular gene's undegraded | PS-HKR | | | |
| | mRNA nucleotide length. | | | | |
| (22) | Compare directly in the microarray | PAFR | — | NP | — |
| | assay hybridization solution labeled | — | — | — | — |
| | mRNA L-LPNs produced from | | | | |
| | (a) Cell sample T-RNA | | | | |
| | (b) Cell sample isolated MRNA | | | | |
| (23) | Labeled mRNA L-LPN nucleotide | — | — | — | — |
| | lengths in a cell sample mRNA L-LPN | | | | |
| | prep are | | | | |
| | (a) The same | | | | |
| | (b) Different | | | | |
| (24) | The average nucleotide lengths of | MLDR* | — | =1 | — |
| | compared cell sample mRNA L-LPN | PL-HKR* | — | — | — |
| | preps are | PS-HKR* | | | |
| | (a) The same | — | | | |
| | (b) Different | | | | |
| (25) | Compared cell sample mRNA L-LPN | MLDR* | — | =1 | — |
| | preps are adjusted to have nucleotide | PS-HKR* | — | =1 | — |
| | lengths which are somewhat longer | PL-HKR* | | | |
| | than the longest CDP on the | | | | |
| | microarray, and which has | | | | |
| | (a) The same or nearly the same | | | | |
| | average nucleotide | | | | |
| | lengths | | | | |
| | (b) Much smaller average nucleotide | | | | |
| | lengths than in | | | | |
| | (a), which are the same | | | | |
| (26) | mRNA L-LPN nucleotide lengths for | MLDR* | — | =1 | |
| | compared particular gene mRNA L- | PL-HKR* | | — | — |
| | LPNs are | PS-HKR* | | | |
| | (a) The same | — | | | |
| | (b) Different | | | | |
| (27) | mRNA L-LPN nucleotide lengths and | MLDR | — | =1 | — |
| | nucleotide sequences are the same or | PL-HKR | | | |
| | essentially the same for all compared | PS-HKR | | | |
| | particular gene mRNA L-LPNs in the | | | | |
| | assay. | | | | |
| (28) | mRNA L-LPN nucleotide lengths and | MLDR | — | =1 | — |
| | nucleotide sequences are the same or | PL-HKR | | | |
| | nearly the same for less than all | PS-HKR | | | |
| | compared particular gene mRNA L- | | | | |
| | LPNs in the assay. | | | | |
| (29) | Compare particular gene undegraded | MLDR | — | =1 | — |
| | labeled mRNA L-LPNs. | PL-HKR | | | |
| | | PS-HKR | | | |
| (30) | For all particular gene comparisons of | SCR | — | =1 | — |
| | labeled mRNA L-LPNs, or cDNA L- | LLSR | | | |
| | LPNs, or cRNA L-LPNs, the assay | SBNR | | | |
| | value for the UNF | SSAR | | | |
| | (a) SCR | | | | |
| | (b) LLSR | | | | |
| | (c) SBNR | | | | |
| | (d) SSAR | | | | |
| | is known to equal one. | | | | |
| (31) | Determine for each particular gene L- | — | — | — | — |
| | LPN comparison the assay value for | | | | |
| | one or more of the UNFs | | | | |
| | (a) MLDR | | | | |
| | (b) PL-HKR | | | | |
| | (c) PS-HKR | | | | |
| | (d) SBNR | | | | |
| | (e) SSAR | | | | |
| | (f) LLSR | | | | |
| (32) | Each of the oligo dT or SG primed | MLDR | — | =1 | — |
| | cDNA, or cRNA, or mRNA, compared | PL-HKR | | | |
| | cell sample L-LPN preps, has an | PS-HKR | | | |
| | average nucleotide length which is | | | | |
| | greater than the nucleotide length of | | | | |
| | undegraded mRNA molecules for one | | | | |
| | or more, but not all, different particular | | | | |
| | genes in the assay. | | | | |
| (33) | Use a cDNA microarray which | — | — | — | — |
| | contains only one CDP sequence for | | | | |
| | each different gene mRNA to be | | | | |
| | detected, and each such particular gene | | | | |
| | CDP sequence has a nucleotide length | | | | |
| | and nucleotide complexity which is | | | | |
| | equal to or preferably, significantly | | | | |
| | shorter than, the nucleotide length or | | | | |
| | complexity of the shortest gene | | | | |
| | undegraded mRNA in the assay. | | | | |
| (34) | Maximize the number of different | All that | All that | — | — |
| | pertinent UNFs and CNFs which have | equal one | equal one | | |
| | an assay value equal to one or nearly | | | | |
| | one. | | | | |
| (35) | The L-LPN ligand label densities of | — | — | — | — |
| | the compared particular gene L-LPNs | | | | |
| | are | | | | |
| | (a) Essentially the same | | | | |
| | (b) Significantly different | | | | |

*Can ignore these UNFs when compared L-LPNs are produced from cell sample T-RNA, but may not be able to ignore these UNFs when the compared L-LPNs are produced from cell sample isolated mRNAs.

Certain of these design solutions have been discussed in the previous section, and others will be discussed and further defined below, while others are self-explanatory. Design Solutions 1, 2, and 3. These were discussed earlier. Note that all of the particular genes on the Affymetrix array, and a very small fraction of the particular genes on the ABI array, are represented by multiple CDP spots on the array. Generally, each gene on an Affymetrix array is represented by 10-20 different CDP gene subsequences. Each gene subsequence CDP represents a different portion of the particular gene mRNA. The assay measured RAS signal for a particular gene is the average of all of the gene subsequence associated RAS values for the gene. Design Solutions 7, 9-12, 14-34. These were previously discussed. Design Solution 13. One ligand refers to using the same ligand to label each compared cell sample L-LPN, and two arrays and two separate hybridization reactions are required for each comparative assay, and the same SGC molecule type is used to stain each array. Two ligands refers to using a different ligand for each compared L-LPN prep, and only one array and one hybridization reaction, which contains both compared L-LPN preps, is required for each comparative assay, and two different SGC molecule types, each specific for only one ligand, are used in the staining step.

The design solutions of Table 74 for the microarray indirect label assays are very similar to those design solutions presented in Table 53 for microarray direct label assays. The NFs associated with the direct and indirect label type 1 LPN assays are the same expect that the UNFs SBNR and SSAR are not associated with type 1 direct label assays, but are associated with type 1 indirect label assays, while the UNFs PSAR and PSSR are not associated with the type 1 indirect label assays, and the UNFs SBNR and SSAR are not associated with type 1 direct label assays. The NFs associated with either direct or indirect label type 1 LPN assays are presented in Tables 47 and 70.

Relative to prior art normalization practice, the normalization of microarray measured particular gene comparison L-LPN assay results is improved when one or more particular gene comparison RASR values produced by such an assay is known to be validly normalized for one or more of the following. (i) one or more pertinent UNFs. (ii) one or more pertinent CNFs. (iii) one or more pertinent UNFs and one or more pertinent CNFs. (iv) one or more pertinent UNFs and all pertinent CNFs. (v) all pertinent CNFs. (vi) all pertinent UNFs. (vii) all pertinent UNFs and all pertinent CNFs. For a microarray L-LPN comparison assay, a preferred improved normalization process assay design solution combination results in the valid normalization of all particular gene L-LPN comparison RASR values in an assay for all pertinent UNFs and CNFs, and also results in minimizing the number of UNF and CNF related false negative results which are associated with the assay. Such preferred assay designs are described below. A variety of different general L-LPN assay designs are practiced by the prior art, and each of these different general assay designs can be associated with a different combination of pertinent UNFs and CNFs. This is illustrated in Tables 70, 71 and 72. Certain of these prior art assay designs are associated with pertinent UNFs, such as PAFR, whose assay values cannot be practically determined for each particular gene comparison in an assay, or the SBNR whose assay values cannot practically be determined for each particular gene comparison in certain assays, or the PL-HKR and PS-HKR whose assay values cannot currently be determined, due to lack of information which is currently unknown. Therefore some prior art general assay designs cannot be modified to allow the improved normalization for each pertinent UNFs and CNFs. This was discussed earlier and illustrated for direct label microarray assays in Tables 64 through 68. Each different prior art general assay design will be discussed initially in terms of the Table 74 design solution combinations which can be known to allow the improved normalization of all or essentially all particular gene comparison RASR values in the assay for the maximum number of assay pertinent UNFs and CNFs. These preferred practice design solution combinations are presented in Tables 75 through 81.

TABLE 75
Preferred Practice for Design Solution Combinations Which Can Be Known to
Completely Normalize All, or Essentially All, Microarray L-LPN Assay Measured
Particular Gene RASR Values for All Pertinent UNFs and CNFs: Compared
Indirectly Labeled mRNAs Produced from T-RNAs

| | | NFs | | Pertinent | |
|---|---|---|---|---|---|
| | | Which Can Be | | NFs To Be | |
| | | Ignored For | | Determined and | |
| | | Normalization | | Normalized For | |
| Combination of Assay Design Solutions | | UNF | CNF | UNF | CNF |
| | Compare Undegraded T-RNA Type 1 | SCR | C-HKR | SCR | Spatial |
| | mRNA LPNs | PAFR | | SBNR | Print Tip |
| (1) | Combine Table 74 Design Solutions | MLDR | | SSAR | Print Plate |
| | (a) 1, or 2, or 3, 4a or b, 5a, 6, 8a, c, | PL-HKR | | | Intensity |
| | d, 13b, 14, 22a, 27, 29, 30a, c, d, | PS-HKR | | | Scale |
| | 34, 35a | SSAR | SBNR | | |
| | or | LLSR | | | |
| | (b) As (1a), except use Design | | | | |
| | Solution 7 instead of Design | | | | |
| | Solution 6 | | | | |
| | or | | | | |
| | (c) As (1a-b), except delete Design | | | | |
| | Solution 8a | | | | |
| | or | | | | |
| | (d) As (1a-c), except use Design | | | | |
| | Solution 25a or 25b | | | | |
| (2) | Combine Table 74 Design Solutions | PAFR | C-HKR | SCR | Spatial |
| | (a) As (1a-d), except delete Design | MLDR | | SBNR | Print Tip |
| | Solution, 30a, c, d, and use Design | PL-HKR | | SSAR | Print Plate |
| | Solution 35a or 35b | PS-HKR | | | Intensity |
| | | LLSR | | | Scale |
| (3) | Combine Table 74 Design Solutions | SCR | — | SCR | C-HKR |
| | (a) 1, or 2, or 3, 4a or b, 5a, 6, 8a, d, | PAFR | | SSAR | Spatial |
| | 13a, 14, 22a, 27, 29, 30a, c, d, 34, | MLDR | | | Print Tip |
| | 35a | PL-HKR | | | Print Plate |
| | or | PS-HKR | | | Intensity |
| | (b) As (3a), except use Design | SBNR | | | Scale |
| | Solution 7 instead of Design | SSAR | | | |
| | Solution 6 | LLSR | | | |
| | or | | | | |
| | (c) As (3a-b), except delete Design | | | | |
| | Solution 8a | | | | |
| | or | | | | |
| | (d) As (3a-c), except use Design | | | | |
| | Solution 25b | | | | |
| (4) | Combine Table 74 Design Solutions | PAFR | — | SCR | C-HKR |
| | As (3a-d), except delete Design | MLDR | | SSAR | Spatial |
| | Solution 30a, d | PL-HKR | | | Print Tip |
| | | PS-HKR | | | Print Plate |
| | | SBNR | | | Intensity |
| | | LLSR | | | Scale |
| | Compare Degraded T-RNA Type 1 | SCR | C-HKR | SCR | Spatial |
| | mRNA L-LPNs | PAFR | | SBNR | Print Tip |
| (5) | Combine Table 74 Design Solutions | MLDR | | SSAR | Print Plate |
| | (a) 1, or 2, or 3, 4a or b, 5a, 6, 8a, c, | PL-HKR | | | Intensity |
| | d, 13b, 14, 22a, 24a, 26a, 27, 30a, c, | PS-HKR | | | Scale |
| | d, 34, 35a | SBNR | | | |
| | or | SSAR | | | |
| | (b) As (5a), except use Design | LLSR | | | |
| | Solution 7 instead of Design | | | | |
| | Solution 6 | | | | |
| | or | | | | |
| | (c) As (5a-b), except delete Design | | | | |
| | Solution 8a | | | | |
| | or | | | | |
| | (d) As (5a-c), except delete Design | | | | |
| | Solutions 24a and 26a and use Design | | | | |
| | Solutions 24b and 26b, and 25a or | | | | |
| | 25b | | | | |
| (6) | Combine Table 74 Design Solutions | PAFR | C-HKR | SCR | Spatial |
| | (a) As (5a-d), except delete Design | MLDR | | SBNR | Print Tip |
| | Solutions 30a, c, d, and use Design | PL-HKR | | SSAR | Print Plate |
| | Solution 35a or 35b | PS-HKR | | | Intensity |
| | | LLSR | | | Scale |
| (7) | Combine Table 74 Design Solutions | SCR | — | SCR | C-HKR |
| | (a) 1, or 2, or 3, 4a or b, 5a, 6, 8a, c, | PAFR | | SSAR | Spatial |
| | d, 13a, 14, 22a, 24a, 26a, 27, 30a, c, | MLDR | | | Print Tip |
| | d, 34, 35a | PL-HKR | | | Print Plate |
| | or | PS-HKR | | | Intensity |
| | (b) As (5a), except use Design | SBNR | | | Scale |
| | Solution 7 instead of Design | SSAR | | | |
| | Solution 6 | LLSR | | | |
| | or | | | | |
| | (c) As (5a-b), except delete Design | | | | |
| | Solution 8a | | | | |
| | or | | | | |
| | (d) As (5a-c), except delete Design | | | | |
| | Solutions 24a and 26a and use Design | | | | |
| | Solutions 24b and 26b, and 25a or | | | | |
| | 25b | | | | |
| (8) | Combine Table 74 Design Solutions | PAFR | — | SCR | C-HKR |
| | (a) As (7a-d), except delete Design | MLDR | | SSAR | Spatial |
| | Solutions 30a, d | PL-HKR | | | Print Tip |
| | | PS-HKR | | | Print Plate |
| | | SBNR | | | Intensity |
| | | LLSR | | | Scale |
| | Compare Undegraded T-RNA Type 2 | SCR | — | SCR | Spatial |
| | mRNA L-LPNs | PAFR | | SSAR | Print Tip |
| (9) | Combine Table 74 Design Solutions | MLDR | | | Print Plate |
| | (a) 1, or 2, or 3, 4a or b, 5b, 6, 8a, b, | PL-HKR | | | Intensity |
| | 13b, 14, 22a, 27, 29, 30a, b, 34, 35a | PS-HKR | | | Scale |
| | or | SBNR | | | |
| | (b) As (9a), except use Design | SSAR | | | |
| | Solution 7 instead of Design | LLSR | | | |
| | Solution 6 | | | | |
| | or | | | | |
| | (c) As (9a-b), except delete Design | | | | |
| | Solution 8a | | | | |
| | or | | | | |
| | (d) As (9a-c), except delete Design | | | | |
| | Solution 25a or 25b | | | | |
| (10) | Combine Table 74 Design Solutions | PAFR | C-HKR | SCR | Spatial |
| | (a) As (9a-d), except delete Design | MLDR | | LLSR | Print Tip |
| | Solutions 30a, b | PL-HKR | | | Print Plate |
| | | PS-HKR | | | Intensity |
| | | SBNR | | | Scale |
| | | SSAR | | | |
| (11) | Combine Table 74 Design Solutions | SCR | — | SCR | C-HKR |
| | (a) 1, or 2, or 3, 4a or b, 5b, 6, 8a, | PAFR | | LLSR | Spatial |
| | b, 13a, 14, 22a, 27, 29, 30a, b, 34, 35a | MLDR | | | Print Tip |
| | or | PL-HKR | | | Print Plate |
| | (b) As (11a), except use Design | PS-HKR | | | Intensity |
| | Solution 7 instead of Design Solution 6 | SBNR | | | Scale |
| | or | SSAR | | | |
| | (c) As (11a-b), except delete | LLSR | | | |
| | Design Solution 8a | | | | |
| | or | | | | |
| | (d) As (11a-c), | | | | |
| | except use Design | | | | |
| | Solution 25b | | | | |
| (12) | Combine Table 74 Design Solutions | PAFR | — | SCR | C-HKR |
| | (a) As (11a-d), except delete | MLDR | | LLSR | Spatial |
| | Design Solutions 30a, b | PL-HKR | | | Print Tip |
| | | PS-HKR | | | Print Plate |
| | | SBNR | | | Intensity |
| | | SSAR | | | Scale |
| | Compare Degraded T-RNA Type 2 | SCR | C-HKR | SCR | Spatial |
| | mRNA LPNs | PAFR | | LLSR | Print Tip |
| (13) | Combine Table 74 Design Solutions | MLDR | | | Print Plate |
| | (a) 1, or 2, or 3, 4a or b, 5b, 6, 8a, | PL-HKR | | | Intensity |
| | b, 13b, 14, 22a, 24a, 26a, 27, 30a, b, | PS-HKR | | | Scale |
| | 34, 35a, or | SBNR | | | |
| | (b) As (13a), except use Design | SSAR | | | |
| | Solution 7 instead of Design Solution | LLSR | | | |
| | 6, or | | | | |
| | (c) As (13a-b), except delete | | | | |
| | Design Solution 8a, or | | | | |
| | (d) As (13a-c), except delete Design | | | | |
| | Solution 24a and 26a, and use | | | | |
| | Design Solutions 24b and 26b, | | | | |
| | and 25a or 25b | | | | |
| (14) | Combine Table 74 Design Solutions | PAFR | C-HKR | SCR | Spatial |
| | (a) As (13a-d), except delete | MLDR | | LLSR | Print Tip |
| | Design Solutions 30a, b | PL-HKR | | | Print Plate |
| | | PS-HKR | | | Intensity |
| | | SBNR | | | Scale |
| | | SSAR | | | |
| (15) | Combine Table 74 Design Solutions | SCR | — | SCR | C-HKR |
| | (a) 1, or 2, or 3, 4a or b, 5b, 6, 8a, | PAFR | | LLSR | Spatial |
| | b, 13a, 14, 22a, 24a, 26a, 27, 30a, b, | MLDR | | | Print Tip |
| | 34, 35a, or | PL-HKR | | | Print Plate |
| | (b) As (15a), except use Design | PS-HKR | | | Intensity |
| | Solution 7 instead of Design Solution | SBNR | | | Scale |
| | 6, or | SSAR | | | |
| | (c) As (15a-b), except delete | LLSR | | | |
| | Design Solution 8a, or | | | | |
| | (d) As (15a-c), except delete Design | | | | |
| | Solution 24a and 26a, and use | | | | |
| | Design Solutions 24b and 26b, | | | | |
| | and 25a or 25b | | | | |
| (16) | Combine Table 74 Design Solutions | PAFR | — | SCR | C-HKR |
| | (a) As (15a-d), except delete | MLDR | | LLSR | Spatial |
| | Design Solutions 30a, b | PL-HKR | | | Print Tip |
| | | PS-HKR | | | Print Plate |
| | | SBNR | | | Intensity |
| | | SSAR | | | Scale |
| | | LLSR | | | |
| (17) | See Table 8 (17). | | | | |

TABLE 76
Preferred Practices for Design Solution Combinations Which Can Be Known to
Completely Normalize All, or Essentially All, Microarray L-LPN Assay Measured
Particular Gene RASR Values for All Pertinent UNFs and CNFs: Comparison of
Specific Gene (SG) Primed Indirectly labeled L-LPNs

| | | NFs Which Can | | Pertinent NFs To Be | |
|---|---|---|---|---|---|
| | | Be Ignored For | | Determined and | |
| | | Normalization | | Normalized For | |
| | Combination of Assay Design Solutions | UNF | CNF | UNF | CNF |
| | Compare Undegraded Type 1 L-LPNs | SCR | C-HKR | SCR | Spatial |
| | From Undegraded T-RNA | PAFR | | SBNR | Print Tip |
| (1) | Combine Table 74 Design Solutions | MLDR | | SSAR | Print Plate |
| | (a) 1, or 2, or 3, 4a or b, 5a, 6, 8a, c, | PL-HKR | | | Intensity |
| | d, 10a, 13b, 14, 19, 21, 30a, c, d, | PS-HKR | | | Scale |
| | 34, 35a | SBNR | | | |
| | or | SSAR | | | |
| | (b) As (1a), except use Design | LLSR | | | |
| | Solution 7 instead of Design | | | | |
| | Solution 6 | | | | |
| | or | | | | |
| | (c) As (1a-b), except delete Design | | | | |
| | Solution 8a | | | | |
| | or | | | | |
| | (d) As (1a-c), except use Design | | | | |
| | Solution 17a or 17b | | | | |
| (2) | Combine Table 74 Design Solutions | PAFR | C-HKR | SCR | Spatial |
| | (a) As (1a-d), except delete Design | MLDR | | SBNR | Print Tip |
| | Solution, 30a, c, d, and use Design | PL-HKR | | SSAR | Print Plate |
| | Solution 35a or 35b | PS-HKR | | | Intensity |
| | | LLSR | | | Scale |
| (3) | Combine Table 74 Design Solutions | SCR | — | SCR | C-HKR |
| | (a) 1, or 2, or 3, 4a or b, 5a, 6, 8a, c, | PAFR | | SSAR | Spatial |
| | d, 10a, 13a, 14, 19, 21, 30a, c, d, | MLDR | | | Print Tip |
| | 34, 35a | PL-HKR | | | Print Plate |
| | or | PS-HKR | | | Intensity |
| | (b) As (3a), except use Design | SBNR | | | Scale |
| | Solution 7 instead of Design | SSAR | | | |
| | Solution 6 | LLSR | | | |
| | or | | | | |
| | (c) As (3a-b), except delete Design | | | | |
| | Solution 8a | | | | |
| | or | | | | |
| | (d) As (3a-c), except use Design | | | | |
| | Solution 17a or 17b | | | | |
| (4) | Combine Table 74 Design Solutions | PAFR | — | SCR | C-HKR |
| | As (3a-d), except delete Design | MLDR | | SSAR | Spatial |
| | Solution 30a, d | PL-HKR | | | Print Tip |
| | | PS-HKR | | | Print Plate |
| | | SBNR | | | Intensity |
| | | LLSR | | | Scale |
| | Comparison of Type 1 L-LPNs Produced | SCR | C-HKR | SCR | Spatial |
| | From T-RNAs | PAFR | | SBNR | Print Tip |
| (5) | Combine Table 74 Design Solutions | MLDR | | SSAR | Print Plate |
| | (a) 1, or 2, or 3, 4a or b, 5a, 6, 8a, c, | PL-HKR | | | Intensity |
| | d, 10a, 13b, 14, 15a, 16a, 18a, 19, | PS-HKR | | | Scale |
| | 30a, c, d, 34, 35a, or | SBNR | | | |
| | (b) As (5a), except use Design | SSAR | | | |
| | Solution 15b instead of Design | LLSR | | | |
| | Solution 15a, or | | | | |
| | (c) As (5a-b), except use Design | | | | |
| | Solution 7 instead of Design Solution | | | | |
| | 6, or | | | | |
| | (d) As (5a-c), except delete Design | | | | |
| | Solution 8a, or | | | | |
| | (e) As (5a-d), except delete Design | | | | |
| | Solutions 16a and 18a, and use | | | | |
| | Design Solutions 16b and 18b, and | | | | |
| | Design Solution 17a or 17b, or | | | | |
| | (f) As (5a-d), except delete Design | | | | |
| | Solutions 1, 3, 16a, and 18a, and | | | | |
| | use Design Solutions 16b and | | | | |
| | 18b, and Design Solutions 17a or | | | | |
| | 17b and Design Solutions 2 or 33 | | | | |
| (6) | Combine Table 74 Design Solutions | PAFR | C-HKR | SCR | Spatial |
| | (a) As (5a-f), except delete Design | MLDR | | SBNR | Print Tip |
| | Solutions 30a, c, d, and use Design | PL-HKR | | SSAR | Print Plate |
| | Solution 35a or 35b | PS-HKR | | | Intensity |
| | | LLSR | | | Scale |
| (7) | Combine Table 74 Design Solutions | SCR | — | SCR | C-HKR |
| | (a) As (5a-f), except use Design | PAFR | | SSAR | Spatial |
| | Solution 13a instead of Design | MLDR | | | Print Tip |
| | Solution 13b | PL-HKR | | | Print Plate |
| | | PS-HKR | | | Intensity |
| | | SBNR | | | Scale |
| | | SSAR | | | |
| | | LLSR | | | |
| (8) | Combine Table 74 Design Solutions | PAFR | — | SCR | C-HKR |
| | (a) As (7a), except delete Design | MLDR | — | SSAR | Spatial |
| | Solutions 30a, d | PL-HKR | | | Print Tip |
| | | PS-HKR | | | Print Plate |
| | | SBNR | | | Intensity |
| | | LLSR | | | Scale |
| | Comparison of Undegraded T-RNA Type | SCR | C-HKR | SCR | Spatial |
| | 2 L-LPNs | PAFR | | LLSR | Print Tip |
| (9) | Combine Table 74 Design Solutions | MLDR | | | Print Plate |
| | (a) 1, or 2, or 3, 4a or b, 5b, 6, 8a, b, | PL-HKR | | | Intensity |
| | 10a, 13b, 14, 15b, 18a, 19, 21, 30a, b, | PS-HKR | | | Scale |
| | 34, 35a | SBNR | | | |
| | or | SSAR | | | |
| | (b) As (9a), except use Design | LLSR | | | |
| | Solution 7 instead of Design | | | | |
| | Solution 6 | | | | |
| | or | | | | |
| | (c) As (9a-b), except delete Design | | | | |
| | Solution 8a | | | | |
| | or | | | | |
| | (d) As (9a-c), except use Design | | | | |
| | Solution 17a or 17b | | | | |
| (10) | Combine Table 74 Design Solutions | PAFR | C-HKR | SCR | Spatial |
| | (a) As (9a-d), except delete Design | MLDR | | LLSR | Print Tip |
| | Solutions 30a, b | PL-HKR | | | Print Plate |
| | | PS-HKR | | | Intensity |
| | | SBNR | | | Scale |
| | | SSAR | | | |
| (11) | Combine Table 74 Design Solutions | SCR | — | SCR | C-HKR |
| | (a) As (9a-d), except use Design | PAFR | | LLSR | Spatial |
| | Solution 13a instead of Design | MLDR | | | Print Tip |
| | Solution 13b | PL-HKR | | | Print Plate |
| | | PS-HKR | | | Intensity |
| | | SBNR | | | Scale |
| | | SSAR | | | |
| | | LLSR | | | |
| (12) | Combine Table 74 Design Solutions | PAFR | — | SCR | C-HKR |
| | (a) As (11a), except delete Design | MLDR | | LLSR | Spatial |
| | Solutions 30a, b | PL-HKR | | | Print Tip |
| | | SBNR | | | Print Plate |
| | | SSAR | | | Intensity |
| | | | | | Scale |
| | Comparison of Type 2 LPNs Produced | SCR | C-HKR | SCR | Spatial |
| | From T-RNAs | PAFR | | LLSR | Print Tip |
| (13) | Combine Table 74 Design Solutions | MLDR | | | Print Plate |
| | (a) 1, or 2, or 3, 4a or b, 5b, 6, 8a, | PL-HKR | | | Intensity |
| | b, 10a, 13b, 14, 15a, 16a, 18a, 19, | PS-HKR | | | Scale |
| | 30a, b, 34, 35a, or | SBNR | | | |
| | (b) As (13a), except use Design | SSAR | | | |
| | Solution 15b instead of Design | LLSR | | | |
| | Solution 15a, or | | | | |
| | (c) As (13a-b), except use Design | | | | |
| | Solution 7 instead of Design Solution | | | | |
| | 6, or | | | | |
| | (d) As (13a-c), except delete Design | | | | |
| | Solution 8a, or | | | | |
| | (e) As (13a-d), except delete | | | | |
| | Design Solutions 16a and 18a | | | | |
| | and use Design Solutions 16b, | | | | |
| | 18b, and Design Solution 17a or | | | | |
| | 17b | | | | |
| (14) | Combine Table 74 Design Solutions | PAFR | C-HKR | SCR | Spatial |
| | (a) As (13a-e), except delete Design | MLDR | | LLSR | Print Tip |
| | Solutions 30a, b | PL-HKR | | | Print Plate |
| | | PS-HKR | | | Intensity |
| | | SBNR | | | Scale |
| | | SSAR | | | |
| (15) | Combine Table 74 Design Solutions | SCR | — | SCR | C-HKR |
| | (a) As (13a-e), except use Design | PAFR | | LLSR | Spatial |
| | Solution 13a | MLDR | | | Print Tip |
| | instead of Design Solution 13b | PL-HKR | | | Print Plate |
| | | PS-HKR | | | Intensity |
| | | SBNR | | | Scale |
| | | SSAR | | | |
| | | LLSR | | | |
| (16) | Combine Table 74 Design Solutions | PAFR | — | SCR | C-HKR |
| | (a) As (15a), except delete Design | MLDR | | LLSR | Spatial |
| | Solutions 30a, b | PL-HKR | | | Print Tip |
| | | PS-HKR | | | Print Plate |
| | | SBNR | | | Intensity |
| | | SSAR | | | Scale |

TABLE 77
Preferred Practice for Design Solution Combinations Which Can Be Known to
Completely Normalize All, or Essentially All, Microarray L-LPN Assay Measured
Particular Gene RASR Values for All Pertinent UNFs and CNFs: Comparison of
Indirectly Labeled Random Primed L-LPNs Produced from T-RNAs

| | | NFs Which Can | | Pertinent NFs To Be | |
|---|---|---|---|---|---|
| | | Be Ignored For | | Determined and | |
| | | Normalization | | Normalized For | |
| Combination of Assay Design Solutions | | UNF | CNF | UNF | CNF |
| | Compare Type 1 LPNs Produced From T- | SCR | C-HKR | SCR | Spatial |
| | RNAs | PAFR | | SBNR | Print Tip |
| (1) | Combine Table 74 Design Solutions | MLDR | | SSAR | Print Plate |
| | (a) 1, or 2, or 3, 4a or b, 5a, 6, 8a, c, | PL-HKR | | | Intensity |
| | d, 11, 13b, 14, 15b, 16a, 18a, 19, 30a, | PS-HKR | | | Scale |
| | c, d, 34, 35a, or | SBNR | | | |
| | (b) As (1a), except use Design | SSAR | | | |
| | Solution 7 instead of Design | LLSR | | | |
| | Solution 6, or | | | | |
| | (c) As (1a-b), except delete Design | | | | |
| | Solution 8a, or | | | | |
| | (d) As (1a-c), delete Design | | | | |
| | Solutions 16a and 18a, and use | | | | |
| | Design Solutions 16b and 18b, and | | | | |
| | 17a or 17b | | | | |
| (2) | Combine Table 74 Design Solutions | PAFR | C-HKR | SCR | Spatial |
| | (a) As (1a-d), except delete Design | MLDR | | SBNR | Print Tip |
| | Solutions 30a, c, d and use | PL-HKR | | SSAR | Print Plate |
| | Design Solution 35a or 35b | PS-HKR | | | Intensity |
| | | LLSR | | | Scale |
| (3) | Combine Table 74 Design Solutions | SCR | — | SCR | C-HKR |
| | (a) 1, or 2, or 3, 4a or b, 5a, 6, 8a, c, | PAFR | | SSAR | Spatial |
| | d, 11, 13a, 14, 15b, 16a, 18a, 19, 30a, | MLDR | | | Print Tip |
| | c, d, 34, 35a, or | PL-HKR | | | Print Plate |
| | (b) As (3a), except use Design | PS-HKR | | | Intensity |
| | Solution 7 instead of Design Solution | SBNR | | | Scale |
| | 6, or | SSAR | | | |
| | (c) As (3a-b), except delete Design | LLSR | | | |
| | Solution 8a, or | | | | |
| | (d) As (3a-c), except delete Design | | | | |
| | Solutions 16a and 18a, and use | | | | |
| | Design Solutions 16b and 18b, and | | | | |
| | 17a or 17b | | | | |
| (4) | Combine Table 74 Design Solutions | PAFR | — | SCR | C-HKR |
| | (a) As (3a-d), except delete | MLDR | | SSAR | Spatial |
| | Design Solutions 30a, d | PL-HKR | | | Print Tip |
| | | PS-HKR | | | Print Plate |
| | | LLSR | | | Intensity |
| | | SBNR | | | Scale |

TABLE 78
Peferred Practices for Design Solution Combinations Which Can Be Known to
Provide Improved Normalization for Pertinent UNFs and/or CNFs for All, or
Essentially All, Microarray Measured Particular Gene RASR Values in An Assay:
Comparison of Oligo dT Primed Indirectly Labeled L-LPNs Produced from T-RNAs
or Isolated mRNAs

| | | NFs Which Can | | Pertinent NFs To Be | |
|---|---|---|---|---|---|
| | | Be Ignored For | | Determined and | |
| | | Normalization | | Normalized For | |
| Combination of Assay Design Solutions | | UNF | CNF | UNF | CNF |
| | Compare Undegraded RNA Type 1 L- | SCR | C-HKR | PAFR | Spatial |
| | LPNs | MLDR | | SCR | Print Tip |
| (1) | Combine Table 74 Design Solutions | PL-HKR | | SBNR | Print Plate |
| | (a) 1, or 2, or 3, 4a or b, 5a, 6, 8a, c, | PS-HKR | | SSAR | Intensity |
| | d, 9a or b, 13b, 14, 18a, 19, 21, | SBNR | | | Scale |
| | 30a, c, d, 34, 35a, or | SSAR | | | |
| | (b) As (1a), except use Design | LLSR | | | |
| | Solution 7 instead of Design | | | | |
| | Solution 6, or | | | | |
| | (c) As (1a-b), except delete Design | | | | |
| | Solution 8a, or | | | | |
| | (d) As (1a-c), except use Design | | | | |
| | Solution 17a or 17b | | | | |
| (2) | Combine Table 74 Design Solutions | MLDR | C-HKR | PAFR | Spatial |
| | (a) As (1a-d), except delete Design | PL-HKR | | SCR | Print Tip |
| | Solution, 30a, c, d, and use Design | PS-HKR | | SBNR | Print Plate |
| | Solution 35a or 35b | LLSR | | SSAR | Intensity |
| | | | | | Scale |
| (3) | Combine Table 74 Design Solutions | SCR | — | PAFR | C-HKR |
| | (a) As (1a-d), except use Design | MLDR | | SCR | Spatial |
| | Solution 13a instead of Design | PL-HKR | | SSAR | Print Tip |
| | Solution 13b | PS-HKR | | | Print Plate |
| | | SBNR | | | Intensity |
| | | SSAR | | | Scale |
| | | LLSR | | | |
| (4) | Combine Table 74 Design Solutions | MLDR | — | PAFR | C-HKR |
| | (a) As (3a), except delete Design | PL-HKR | | SCR | Spatial |
| | Solutions 30a, d | PS-HKR | | SSAR | Print Tip |
| | | SBNR | | | Print Plate |
| | | LLSR | | | Intensity |
| | | | | | Scale |
| | Compare Type 1 L-LPNs Produced From | SCR | C-HKR | PAFR | Spatial |
| | T-RNAs or Isolated mRNAs | MLDR | | SCR | Print Tip |
| (5) | Combine Table 74 Design Solutions | PL-HKR | | SBNR | Print Plate |
| | (a) 1, or 2, or 3, 4a or b, 5a, 6, 8a, c, | PS-HKR | | SSAR | Intensity |
| | d, 9a or b, 13b, 14, 15a, 16a, 18a, 19, | SBNR | | | Scale |
| | 30a, c, d, 34, 35a | SSAR | | | |
| | or | LLSR | | | |
| | (b) As (5a), except use Design | | | | |
| | Solution 15b instead of Design | | | | |
| | Solution 15a | | | | |
| | or | | | | |
| | (c) As (5a-b), except use Design | | | | |
| | Solution 7 instead of Design Solution 6 | | | | |
| | or | | | | |
| | (d) As (5a-c), except delete Design | | | | |
| | Solution 8a | | | | |
| | or | | | | |
| | (e) As (5a-d), except delete Design | | | | |
| | Solutions 1, 3, 16a and 18a, and use | | | | |
| | Design Solutions 16b, 18b, and 17a or | | | | |
| | 17b, and Design Solution 2 or 33 | | | | |
| (6) | Combine Table 74 Design Solutions | MLDR | C-HKR | PAFR | Spatial |
| | (a) As (5a-e), except delete Design | PL-HKR | | SCR | Print Tip |
| | Solutions 30a, c, d, and use Design | PS-HKR | | SBNR | Print Plate |
| | Solution 35a or 35b | LLSR | | SSAR | Intensity |
| | | | | | Scale |
| (7) | Combine Table 74 Design Solutions | SCR | — | PAFR | C-HKR |
| | (a) As (5a-e), except use Design | MLDR | | SCR | Spatial |
| | Solution 13a instead of Design | PL-HKR | | SSAR | Print Tip |
| | Solution 13b | PS-HKR | | | Print Plate |
| | | SBNR | | | Intensity |
| | | SSAR | | | Scale |
| | | LLSR | | | |
| (8) | Combine Table 74 Design Solutions | MLDR | — | PAFR | C-HKR |
| | (a) As (7a), except delete Design | PL-HKR | | SCR | Spatial |
| | Solutions 30a, d | PS-HKR | | SSAR | Print Tip |
| | | SBNR | | | Print Plate |
| | | LLSR | | | Intensity |
| | | | | | Scale |
| | Compare Undegraded RNA Type 2 L- | SCR | C-HKR | PAFR | Spatial |
| | LPNs | MLDR | | SCR | Print Tip |
| (9) | Combine Table 74 Design Solutions | PL-HKR | | SSAR | Print Plate |
| | (a) 1, or 2, or 3, 4a or b, 5a, 6, 8a, b, | PS-HKR | | | Intensity |
| | 9a or b, 13b, 14, 18a, 19, 21, 30a, b, | SBNR | | | Scale |
| | 34, 35a, or | SSAR | | | |
| | (b) As (9a), except use Design | LLSR | | | |
| | Solution 7 instead of Design | | | | |
| | Solution 6, or | | | | |
| | (c) As (9a-b), except delete Design | | | | |
| | Solution 8a, or | | | | |
| | (d) As (9a-c), except delete Design | | | | |
| | Solution 17a or 17b | | | | |
| | or | | | | |
| | (c) As (9a-b), except delete Design | | | | |
| | Solution 8a, or | | | | |
| | (d) As (9a-c), except use Design | | | | |
| | Solution 17a or 17b | | | | |
| (10) | Combine Table 74 Design Solutions | MLDR | C-HKR | PAFR | Spatial |
| | (a) As (9a-d), except delete Design | PL-HKR | | SCR | Print Tip |
| | Solutions 30a, b | PS-HKR | | SSAR | Print Plate |
| | | SBNR | | | Intensity |
| | | SSAR | | | Scale |
| (11) | Combine Table 74 Design Solutions | SCR | — | PAFR | C-HKR |
| | (a) As (9a-d), except use Design | MLDR | | SCR | Spatial |
| | Solutions 13a instead of Design | PL-HKR | | SSAR | Print Tip |
| | Solution 13b | PS-HKR | | | Print Plate |
| | | SBNR | | | Intensity |
| | | SSAR | | | Scale |
| | | LLSR | | | |
| (12) | Combine Table 74 Design Solutions | MLDR | — | PAFR | C-HKR |
| | (a) As (11a), except delete Design | PL-HKR | | SCR | Spatial |
| | Solutions 30a, b | PS-HKR | | SSAR | Print Tip |
| | | SBNR | | | Print Plate |
| | | SSAR | | | Intensity |
| | | | | | Scale |
| | Compare Type 2 L-LPNs Produced From | SCR | C-HKR | PAFR | Spatial |
| | T-RNA or Isolated mRNA | MLDR | | SCR | Print Tip |
| (13) | Combine Table 74 Design Solutions | PL-HKR | | SSAR | Print Plate |
| | (a) 1, or 2, or 3, 4a or b, 5b, 6, 8a, | PS-HKR | | | Intensity |
| | b, 9a or b, 13b, 14, 16a, 18a, 19, 30a, | SBNR | | | Scale |
| | b, 34, 35a, or | SSAR | | | |
| | (b) As (13a), except use Design | LLSR | | | |
| | Solution 7 instead of Design Solution | | | | |
| | 6, or | | | | |
| | (c) As (13a-b), except delete | | | | |
| | Design Solution 8a, or | | | | |
| | (d) As (13a-c), except use Design | | | | |
| | Solution 17a or 17b | | | | |
| (14) | Combine Table 74 Design Solutions | MLDR | C-HKR | PAFR | Spatial |
| | (a) As (13a-d), except delete | PL-HKR | | SCR | Print Tip |
| | Design Solutions 30a, b | PS-HKR | | LLSR | Print Plate |
| | | SBNR | | | Intensity |
| | | SSAR | | | Scale |
| (15) | Combine Table 74 Design Solutions | SCR | — | PAFR | C-HKR |
| | (a) As (13a-d), except use Design | MLDR | | SCR | Spatial |
| | Solution 13a instead of Design | PL-HKR | | LLSR | Print Tip |
| | Solution 13b | PS-HKR | | | Print Plate |
| | | SBNR | | | Intensity |
| | | SSAR | | | Scale |
| | | LLSR | | | |
| (16) | Combine Table 74 Design Solutions | MLDR | — | PAFR | C-HKR |
| | (a) As (15a), except delete Design | PL-HKR | | SCR | Spatial |
| | Solutions 30a, b | PS-HKR | | LLSR | Print Tip |
| | | SBNR | | | Print Plate |
| | | SSAR | | | Intensity |
| | | | | | Scale |

TABLE 79
Preferred Practices for Design Solution Combinations Which Can Be Known to
Provide Improved Normalization for Pertinent UNFs and/or CNFs for All, or
Essentially All, Microarray Measured Particular Gene RASR Values in An Assay:
Comparison of Indirectly Labeled Isolated mRNA L-LPNs

| | | NFs Which Can | | Pertinent NFs To Be | |
|---|---|---|---|---|---|
| | | Be Ignored For | | Determined and | |
| | | Normalization | | Normalized For | |
| Combination of Assay Design Solutions | | UNF | CNF | UNF | CNF |
| | Compare Undegraded Type 1 mRNA L- | SCR | C-HKR | PAFR | Spatial |
| | LPNs | MLDR | | SCR | Print Tip |
| (1) | Combine Table 74 Design Solutions | PL-HKR | | SBNR | Print Plate |
| | (a) 1, or 2, or 3, 4a or b, 5a, 6, 8a, c, | PS-HKR | | SSAR | Intensity |
| | d, 13b, 14, 22b, 27, 29, 30a, c, d, | SBNR | | | Scale |
| | 34, 35a, or | SSAR | | | |
| | (b) As (1a), except use Design | LLSR | | | |
| | Solution 7 instead of Design | | | | |
| | Solution 6, or | | | | |
| | (c) As (1a-b), except delete Design | | | | |
| | Solution 8a, or | | | | |
| | (d) As (1a-c), except use Design | | | | |
| | Solution 25b, or | | | | |
| | (e) As (1a-d), except use Design | | | | |
| | Solution 17a or 17b | | | | |
| (2) | Combine Table 74 Design Solutions | MLDR | C-HKR | PAFR | Spatial |
| | (a) As (1a-e), except delete Design | PL-HKR | | SCR | Print Tip |
| | Solution, 30a, c, d, and use Design | PS-HKR | | SBNR | Print Plate |
| | Solution 35a or 35b | LLSR | | SSAR | Intensity |
| | | | | | Scale |
| (3) | Combine Table 74 Design Solutions | SCR | — | PAFR | C-HKR |
| | (a) As (1a-e), except use Design | MLDR | | SCR | Spatial |
| | Solution 13a instead of Design | PL-HKR | | SSAR | Print Tip |
| | Solution 13b | PS-HKR | | | Print Plate |
| | | SBNR | | | Intensity |
| | | SSAR | | | Scale |
| | | LLSR | | | |
| (4) | Combine Table 74 Design Solutions | MLDR | — | PAFR | C-HKR |
| | (a) As (3a), except delete Design | PL-HKR | | SCR | Spatial |
| | Solutions 30a, d | PS-HKR | | SSAR | Print Tip |
| | | SBNR | | | Print Plate |
| | | LLSR | | | Intensity |
| | | | | | Scale |
| | Compare mRNA Type 1 L-LPNs | SCR | C-HKR | PAFR | Spatial |
| | Produced From Isolated mRNAs Which | MLDR | | SCR | Print Tip |
| | Were Produced From Degraded T-RNAs | PL-HKR | | SBNR | Print Plate |
| (5) | Combine Table 74 Design Solutions | PS-HKR | | SSAR | Intensity |
| | (a) 1, or 2, or 3, 4a or b, 5a, 6, 8a, c, | SBNR | | | Scale |
| | d, 13b, 14, 22b, 24a, 26a, 27, 30a, c, | SSAR | | | |
| | d, 34, 35a | LLSR | | | |
| | or | | | | |
| | (b) As (5a), except use Design | | | | |
| | Solution 7 instead of Design | | | | |
| | Solution 6 | | | | |
| | or | | | | |
| | (c) As (5a-b), except delete Design | | | | |
| | Solution 8a | | | | |
| | or | | | | |
| | (d) As (5a-c), except delete Design | | | | |
| | Solutions 1, 3, 24a and 26a, and use | | | | |
| | Design Solutions 24b, 26b, and 25a or | | | | |
| | 25b, and Design Solution 2 or 33 | | | | |
| | or | | | | |
| | (e) As (5a-d), except use Design | | | | |
| | Solution 17a or 17b | | | | |
| | (6) Combine Table 74 Design Solutions | MLDR | C-HKR | PAFR | Spatial |
| | (a) As (5a-d), except delete Design | PL-HKR | | SCR | Print Tip |
| | Solutions 30a, c, d, and use Design | PS-HKR | | SBNR | Print Plate |
| | Solution 35a or 35b | LLSR | | SSAR | Intensity |
| | | | | | Scale |
| (7) | Combine Table 74 Design Solutions | SCR | — | PAFR | C-HKR |
| | (a) As (5a-d), except use Design | MLDR | | SCR | Spatial |
| | Solution 13a instead of Design | PL-HKR | | SSAR | Print Tip |
| | Solution 13b | PS-HKR | | | Print Plate |
| | | SBNR | | | Intensity |
| | | SSAR | | | Scale |
| | | LLSR | | | |
| (8) | Combine Table 74 Design Solutions | MLDR | — | PAFR | C-HKR |
| | (a) As (7a), except delete Design | PL-HKR | | SCR | Spatial |
| | Solutions 30a, d | PS-HKR | | SSAR | Print Tip |
| | | SBNR | | | Print Plate |
| | | LLSR | | | Intensity |
| | | | | | Scale |
| | Compare mRNA Type 1 L-LPNs | SCR | C-HKR | PAFR | Spatial |
| | Produced From Degraded Isolated | MLDR | | SCR | Print Tip |
| | mRNAs Which Became Degraded After | PL-HKR | | SBNR | Print Plate |
| | Isolation From Undegraded T-RNAs | PS-HKR | | SSAR | Intensity |
| (9) | Combine Table 74 Design Solutions | SBNR | | | Scale |
| | (a) As (5a-c, e), except delete Design | SSAR | | | |
| | Solution 25a or 25b | LLSR | | | |
| (10) | Combine Table 74 Design Solutions | SCR | C-HKR | PAFR | Spatial |
| | (a) As (9a), except delete Design | MLDR | | SCR | Print Tip |
| | Solutions 30a, c, d and use Design | PL-HKR | | SBNR | Print Plate |
| | Solution 35a or 35b | PS-HKR | | SSAR | Intensity |
| | | LLSR | | | Scale |
| (11) | Combine Table 74 Design Solutions | SCR | — | PAFR | C-HKR |
| | (a) As (9a), except use Design | MLDR | | SCR | Spatial |
| | Solution 13a instead of Design | PL-HKR | | LLSR | Print Tip |
| | Solution 13b | PS-HKR | | | Print Plate |
| | | SBNR | | | Intensity |
| | | SSAR | | | Scale |
| | | LLSR | | | |
| (12) | Combine Table 74 Design Solutions | MLDR | — | PAFR | C-HKR |
| | (a) As (11a), except delete Design | PL-HKR | | SCR | Spatial |
| | Solutions 30a, d and use Design | PS-HKR | | SSAR | Print Tip |
| | Solution 35a or 35b | SBNR | | | Print Plate |
| | | LLSR | | | Intensity |
| | | | | | Scale |
| | Compare Undegraded Type 2 mRNA L- | SCR | C-HKR | PAFR | Spatial |
| | LPNs | MLDR | | SCR | Print Tip |
| (13) | Combine Table 74 Design Solutions | PL-HKR | | LLSR | Print Plate |
| | (a) 1, or 2, or 3, 4a or b, 5b, 6, 8a, | PS-HKR | | | Intensity |
| | b, 13b, 14, 22b, 26a, 27, 29, 30a, b, | SBNR | | | Scale |
| | 34, 35a, or | SSAR | | | |
| | (b) As (13a), except use Design | LLSR | | | |
| | Solution 7 instead of Design Solution | | | | |
| | 6, or | | | | |
| | (c) As (13a-b), except delete | | | | |
| | Design Solution 8a, or | | | | |
| | (d) As (13a-c), except use Design | | | | |
| | Solution 25a or 25b, or | | | | |
| | (e) As (13a-d), except use Design | | | | |
| | Solution 17a or 17b | | | | |
| (14) | Combine Table 53 Design Solutions | MLDR | C-HKR | PAFR | Spatial |
| | (a) As (13a-e), except delete Design | PL-HKR | | SCR | Print Tip |
| | Solutions 30a, b | PS-HKR | | LLSR | Print Plate |
| | | SBNR | | | Intensity |
| | | SSAR | | | Scale |
| (15) | Combine Table 74 Design Solutions | SCR | — | PAFR | C-HKR |
| | (a) As (13a-e), except use Design | MLDR | | SCR | Print Tip |
| | Solution 13a instead of Design | PL-HKR | | LLSR | Print Plate |
| | Solution 13b | PS-HKR | | | Intensity |
| | | SBNR | | | Scale |
| | | SSAR | | | |
| | | LLSR | | | |
| (16) | Combine Table 74 Design Solutions | MLDR | — | PAFR | C-HKR |
| | (a) As (15a), except delete Design | PL-HKR | | SCR | Spatial |
| | Solutions 30a, b | PS-HKR | | LLSR | Print Tip |
| | and use Design Solution 35a or 35b | SBNR | | | Print Plate |
| | | SSAR | | | Intensity |
| | | | | | Scale |
| | Compare mRNA Type 2 L-LPNs | SCR | C-HKR | PAFR | Spatial |
| | Produced From Degraded Isolated mRNA | MLDR | | SCR | Print Tip |
| (17) | Combine Table 74 Design Solutions | PL-HKR | | LLSR | Print Plate |
| | (a) 1, or 2, or 3, 4a or b, 5b, 6, 8a, | PS-HKR | | | Intensity |
| | b, 13b, 14, 22b, 24a, 26a, 27, 30a, b, | SBNR | | | Scale |
| | 34, 35a | SSAR | | | |
| | or | LLSR | | | |
| | (b) As (17a), except use Design | | | | |
| | Solution 7 instead of Design Solution 6 | | | | |
| | or | | | | |
| | (c) As (17a-b), except delete | | | | |
| | Design Solution 8a | | | | |
| | or | | | | |
| | (d) As (17a-c), except delete Design | | | | |
| | Solutions 24a and 26a, and use | | | | |
| | Design Solutions 24b, 26b, and25a or | | | | |
| | 25b, and Design Solution 17a or 17b | | | | |
| (18) | Combine Table 74 Design Solutions | MLDR | C-HKR | PAFR | Spatial |
| | (a) As (17a-d), except delete | PL-HKR | | SCR | Print Tip |
| | Design Solutions 30a, b | PS-HKR | | LLSR | Print Plate |
| | | SBNR | | | Intensity |
| | | SSAR | | | Scale |
| (19) | Combine Table 74 Design Solutions | SCR | — | PAFR | C-HKR |
| | (a) As (17a-d), except use Design | MLDR | | SCR | Spatial |
| | Solution 13a instead of Design | PL-HKR | | LLSR | Print Tip |
| | Solution 13b | PS-HKR | | | Print Plate |
| | | SBNR | | | Intensity |
| | | SSAR | | | Scale |
| | | LLSR | | | |
| (20) | Combine Table 74 Design Solutions | MLDR | — | PAFR | C-HKR |
| | (a) As (19a), except delete Design | PL-HKR | | SCR | Spatial |
| | Solutions 30a, b | PS-HKR | | LLSR | Print Tip |
| | | SBNR | | | Print Plate |
| | | SSAR | | | Intensity |
| | | | | | Scale |

TABLE 80
Preferred Practices for Design Solution Combinations Which Can Be Known to
Provide Improved Normalization for Pertinent UNFs and/or CNFs for All, or
Essentially All, Microarray Assay Measured Particular Gene RASR Values in An
Assay: Comparison of Indirectly Labeled SG Primed L-LPNs Produced From
Isolated mRNAs

| | | NFs Which Can | | Pertinent NFs To Be | |
|---|---|---|---|---|---|
| | | Be Ignored For | | Determined and | |
| | | Normalization | | Normalized For | |
| Combination of Assay Design Solutions | | UNF | CNF | UNF | CNF |
| | Compare Undegraded Isolated mRNA | SCR | C-HKR | PAFR | Spatial |
| | Type 1 L-LPNs | MLDR | | SCR | Print Tip |
| (1) | Combine Table 74 Design Solutions | PL-HKR | | SBNR | Print Plate |
| | (a) 1, or 2, or 3, 4a or b, 5a, 6, 8a, c, | PS-HKR | | SSAR | Intensity |
| | d, 10b, 13b, 14, 18a, 19, 21, 30a, | SBNR | | | Scale |
| | c, d, 34, 35a | SSAR | | | |
| | or | LLSR | | | |
| | (b) As (1a), except use Design | | | | |
| | Solution 7 instead of Design | | | | |
| | Solution 6 | | | | |
| | or | | | | |
| | (c) As (1a-b), except delete Design | | | | |
| | Solution 8a | | | | |
| | or | | | | |
| | (d) As (1a-c), except use Design | | | | |
| | Solution 17a or 17b | | | | |
| (2) | Combine Table 74 Design Solutions | MLDR | C-HKR | PAFR | Spatial |
| | (a) As (1a-d), except delete Design | PL-HKR | | SCR | Print Tip |
| | Solution, 30a, c, d, and use Design | PS-HKR | | SBNR | Print Plate |
| | Solution 35a or 35b | LLSR | | SSAR | Intensity |
| | | | | | Scale |
| (3) | Combine Table 74 Design Solutions | SCR | C-HKR | PAFR | Spatial |
| | (a) As (1a-d), except use Design | MLDR | | SCR | Print Tip |
| | Solution 13a instead of Design | PL-HKR | | SSAR | Print Plate |
| | Solution 13b | PS-HKR | | | Intensity |
| | | SBNR | | | Scale |
| | | SSAR | | | |
| | | LLSR | | | |
| (4) | Combine Table 74 Design Solutions | MLDR | C-HKR | PAFR | Spatial |
| | (a) As (3a), except delete Design | PL-HKR | | SCR | Print Tip |
| | Solutions 30a, d | PS-HKR | | SSAR | Print Plate |
| | | SBNR | | | Intensity |
| | | LLSR | | | Scale |
| | Compare Type 1 L-LPNs Produced From | SCR | C-HKR | PAFR | Spatial |
| | Degraded Isolated mRNAs | MLDR | | SCR | Print Tip |
| (5) | Combine Table 74 Design Solutions | PL-HKR | | SBNR | Print Plate |
| | (a) 1, or 2, or 3, 4a or b, 5a, 6, 8a, c, | PS-HKR | | SSAR | Intensity |
| | d, 10b, 13b, 14, 15a, 16a, 18a, 19, | SBNR | | | Scale |
| | 30a, c, d, 34, 35a | SSAR | | | |
| | or | LLSR | | | |
| | (b) As (5a), except use Design | | | | |
| | Solution 15b instead of Design | | | | |
| | Solution 15 | | | | |
| | or | | | | |
| | (c) As (5a-b), except use Design | | | | |
| | Solution 7 instead of Design | | | | |
| | Solution 6 | | | | |
| | or | | | | |
| | (d) As (5a-c), except delete Design | | | | |
| | Solution 8a | | | | |
| | or | | | | |
| | (e) As (5a-d), except delete Design | | | | |
| | Solutions 1, 3, 16a and 18a, and use | | | | |
| | Design Solutions 16b, 18b, and 17a or | | | | |
| | 17b, and 2 or 33 | | | | |
| (6) | Combine Table 74 Design Solutions | MLDR | C-HKR | PAFR | Spatial |
| | (a) As (5a-e), except delete Design | PL-HKR | | SCR | Print Tip |
| | Solutions 30a, c, d, and use Design | PS-HKR | | SBNR | Print Plate |
| | Solution 35a or 35b | LLSR | | SSAR | Intensity |
| | | | | | Scale |
| (7) | Combine Table 74 Design Solutions | SCR | — | PAFR | C-HKR |
| | (a) As (5a-e), except use Design | MLDR | | SCR | Spatial |
| | Solution 13a instead of Design | PL-HKR | | SSAR | Print Tip |
| | Solution 13b | PS-HKR | | | Print Plate |
| | | SBNR | | | Intensity |
| | | SSAR | | | Scale |
| | | LLSR | | | |
| (8) | Combine Table 74 Design Solutions | MLDR | — | PAFR | C-HKR |
| | (a) As (7a), except delete Design | PL-HKR | | SCR | Spatial |
| | Solutions 30a, d | PS-HKR | | SSAR | Print Tip |
| | | SBNR | | | Print Plate |
| | | LLSR | | | Intensity |
| | | | | | Scale |
| | Compare Undegraded Isolated mRNA | SCR | C-HKR | PAFR | Spatial |
| | Type 2 L-LPNs | MLDR | | SCR | Print Tip |
| (9) | Combine Table 74 Design Solutions | PL-HKR | | LLSR | Print Plate |
| | (a) 1, or 2, or 3, 4a or b, 5b, 6, 8a, b, | PS-HKR | | | Intensity |
| | 10b, 13b, 14, 18a, 19, 21, 30a, b, 34, | SBNR | | | Scale |
| | 35a | SSAR | | | |
| | or | LLSR | | | |
| | (b) As (9a), except use Design | | | | |
| | Solution 7 instead of Design | | | | |
| | Solution 6 | | | | |
| | or | | | | |
| | (c) As (9a-b), except delete Design | | | | |
| | Solution 8a | | | | |
| | or | | | | |
| | As (9a-c), except use Design Solution | | | | |
| | 17a or 17b | | | | |
| (10) | Combine Table 74 Design Solutions | MLDR | C-HKR | PAFR | Spatial |
| | (a) As (9a-d), except delete Design | PL-HKR | | SCR | Print Tip |
| | Solutions 30a, b | PS-HKR | | SSAR | Print Plate |
| | | SBNR | | | Intensity |
| | | SSAR | | | Scale |
| (11) | Combine Table 74 Design Solutions | SCR | — | PAFR | C-HKR |
| | (a) As (9a-d), except use Design | MLDR | | SCR | Spatial |
| | Solution 13a instead of Design | PL-HKR | | LLSR | Print Tip |
| | Solution 13b | PS-HKR | | | Print Plate |
| | | SBNR | | | Intensity |
| | | SSAR | | | Scale |
| | | LLSR | | | |
| (12) | Combine Table 74 Design Solutions | MLDR | — | PAFR | C-HKR |
| | (a) As (11a), except delete Design | PL-HKR | | SCR | Spatial |
| | Solutions 30a, b | PS-HKR | | SSAR | Print Tip |
| | | SBNR | | | Print Plate |
| | | SSAR | | | Intensity |
| | | | | | Scale |
| | Compare Type 2 L-LPNs Produced From | SCR | C-HKR | PAFR | Spatial |
| | Degraded Isolated mRNAs | MLDR | | SCR | Print Tip |
| (13) | Combine Table 74 Design Solutions | PL-HKR | | LLSR | Print Plate |
| | (a) 1, or 2, or 3, 4a or b, 5b, 6, 8a, | PS-HKR | | | Intensity |
| | b, 10b, 13b, 14, 15a, 16a, 18a, 19, | SBNR | | | Scale |
| | 30a, b, 34, 35a, or | SSAR | | | |
| | (b) As (13a), except use Design | LLSR | | | |
| | Solution 15b instead of Design | | | | |
| | Solution 15a, or | | | | |
| | (c) As (13a-b), except use Design | | | | |
| | Solution 7 instead of Design Solution | | | | |
| | 6, or | | | | |
| | (d) As (13a-c), except delete Design | | | | |
| | Solution 8a, or | | | | |
| | (e) As (13a-d), except delete | | | | |
| | Design Solutions 16a, and 18a, and | | | | |
| | use Design Solutions 16b and 18b, | | | | |
| | and Design Solution 17a or 17b | | | | |
| (14) | Combine Table 74 Design Solutions | MLDR | C-HKR | PAFR | Spatial |
| | (a) As (13a-e), except delete Design | PL-HKR | | SCR | Print Tip |
| | Solutions 30a, b | PS-HKR | | LLSR | Print Plate |
| | | SBNR | | | Intensity |
| | | SSAR | | | Scale |
| (15) | Combine Table 74 Design Solutions | SCR | — | PAFR | C-HKR |
| | (a) As (13a-e), except use Design | MLDR | | SCR | Spatial |
| | Solutions 13a instead of Design | PL-HKR | | LLSR | Print Tip |
| | Solution 13b | PS-HKR | | | Print Plate |
| | | SBNR | | | Intensity |
| | | SSAR | | | Scale |
| | | LLSR | | | |
| (16) | Combine Table 53 Design Solutions | MLDR | — | PAFR | C-HKR |
| | (a) As (15a), except delete Design | PL-HKR | | SCR | Spatial |
| | Solutions 30a, b | PS-HKR | | LLSR | Print Tip |
| | | SBNR | | | Print Plate |
| | | SSAR | | | Intensity |
| | | | | | Scale |

TABLE 81
Preferred Practices for Design Solution Combinations Which Can Be Known to
Provide Improved Normalization for Pertinent UNFs and/or CNFs for All, or
Essentially All, Microarray Assay Measured Particular Gene RASR Values in An
Assay: Comparison of Indirectly Labeled Random Primed L-LPNs Produced From
Isolated mRNA

| | | NFs Which Can | | Pertinent NFs To Be | |
|---|---|---|---|---|---|
| | | Be Ignored For | | Determined and | |
| | | Normalization | | Normalized For | |
| Combination of Assay Design Solutions | | UNF | CNF | UNF | CNF |
| | Compare Type 1 L-LPNs Produced From | SCR | C-HKR | PAFR | Spatial |
| | Undegraded Isolated mRNA or Isolated | MLDR | | SCR | Print Tip |
| | Degraded mRNA Which Became | PL-HKR | | SBNR | Print Plate |
| | Degraded After Isolation | PS-HKR | | SSAR | Intensity |
| (1) | Combine Table 74 Design Solutions | SBNR | | | Scale |
| | (a) 1, or 2, or 3, 4a or b, 5a, 6, 8a, c, | SSAR | | | |
| | d, 12, 13b, 14, 15b, 16a, 18a, 19, | LLSR | | | |
| | 30a, c, d, 34, 35a | | | | |
| | or | | | | |
| | (b) As (1a), except use Design | | | | |
| | Solution 7 instead of Design | | | | |
| | Solution 6 | | | | |
| | or | | | | |
| | (c) As (1a-b), except delete Design | | | | |
| | Solution 8a | | | | |
| | or | | | | |
| | (d) As (1a-c), except delete Design | | | | |
| | Solutions 16a, and 18a, and use | | | | |
| | Design Solutions 16b, 18b, and | | | | |
| | Design Solution 17a or 17b | | | | |
| (2) | Combine Table 74 Design Solutions | MLDR | C-HKR | PAFR | Spatial |
| | (a) As (1a-d), except delete Design | PL-HKR | | SCR | Print Tip |
| | Solution, 30a, c, d, and use Design | PS-HKR | | SBNR | Print Plate |
| | Solution 35a or 35b | LLSR | | SSAR | Intensity |
| | | | | | Scale |
| (3) | Combine Table 74 Design Solutions | SCR | — | PAFR | C-HKR |
| | (a) As (1a-d), except use Design | MLDR | | SCR | Spatial |
| | Solution 13a instead of Design | PL-HKR | | SSAR | Print Tip |
| | Solution 13b | PS-HKR | | | Print Plate |
| | | SBNR | | | Intensity |
| | | SSAR | | | Scale |
| | | LLSR | | | |
| (4) | Combine Table 74 Design Solutions | MLDR | — | PAFR | C-HKR |
| | (a) As (3a), except delete Design | PL-HKR | | SCR | Spatial |
| | Solutions 30a, d | PS-HKR | | SSAR | Print Tip |
| | | SBNR | | | Print Plate |
| | | LLSR | | | Intensity |
| | | | | | Scale |
| | Compare Type 1 L-LPNs Produced From | SCR | C-HKR | PAFR | Spatial |
| | Isolated mRNA Which Was Isolated | MLDR | | SCR | Print Tip |
| | From Degraded T-RNA | PL-HKR | | SBNR | Print Plate |
| (5) | Combine Table 74 Design Solutions | PS-HKR | | SSAR | Intensity |
| | (a) 1, or 2, or 3, 4a or b, 5a, 6, 8a, c, | SBNR | | | Scale |
| | d, 12, 13b, 14, 15b, 16a, 18a, 19, 30a, | SSAR | | | |
| | c, d, 34, 35a | LLSR | | | |
| | or | | | | |
| | (b) As (5a), except use Design | | | | |
| | Solution 7 instead of Design | | | | |
| | Solution 6 | | | | |
| | or | | | | |
| | (c) As (5a-b), except delete Design | | | | |
| | Solution 8a | | | | |
| | or | | | | |
| | (d) As (5a-c), except delete Design | | | | |
| | Solutions 1, 3, 16a, 18a, and use | | | | |
| | Design Solutions 16b and 18b, and 2 | | | | |
| | or 33, and 17a or 17b | | | | |
| (6) | Combine Table 74 Design Solutions | MLDR | C-HKR | PAFR | Spatial |
| | (a) As (5a-d), except delete Design | PL-HKR | | SCR | Print Tip |
| | Solutions 30a, c, d, and use Design | PS-HKR | | SBNR | Print Plate |
| | Solution 35a or 35b | LLSR | | SSAR | Intensity |
| | | | | | Scale |
| (7) | Combine Table 74 Design Solutions | SCR | — | PAFR | C-HKR |
| | (a) As (5a-d), except use Design | MLDR | | SCR | Spatial |
| | Solution 13a instead of Design | PL-HKR | | SSAR | Print Tip |
| | Solution 13b | PS-HKR | | | Print Plate |
| | | SBNR | | | Intensity |
| | | SSAR | | | Scale |
| | | LLSR | | | |
| (8) | Combine Table 74 Design Solutions | MLDR | — | PAFR | C-HKR |
| | (a) As (7a), except delete Design | PL-HKR | | SCR | Spatial |
| | Solutions 30a, d | PS-HKR | | SSAR | Print Tip |
| | | SBNR | | | Print Plate |
| | | LLSR | | | Intensity |
| | | | | | Scale |

Design solution combinations which can be known to provide improved normalization for all or essentially all, particular gene indirect label L-LPN comparison RASR values are presented in Tables 82 through 88. While these design solutions provide improved normalization, they are not considered to be preferred methods because they rely on the determination of PL-HKR, PS-HKR and SBNR for the assay. As discussed, information necessary for the determination of the PL-HKR and PS-HKR UNFs is currently unknown and must be generated. In addition, conditions under which it is not possible to determine the PL-HKR and PS-HKR values, are also conditions where it may not be practical to determine the assay SBNR assay values for the different particular gene comparisons in the assay. Table 89 presents design solution combinations which can be known to more completely normalize only an identifiable subset of particular gene comparison RASR values for pertinent UNFs and CNFs, while less complete improved normalization occurs for all other particular gene comparison RASR values in the L-LPN comparison assay. Table 90 presents design solution combinations which can be known to minimize or eliminate the occurrence of UNF and CNF related particular gene false negative results and their associated RDMs. The design solution combinations presented in Tables 75 through 90 are only a few of large number of different design solution combinations which can be known to provide improved normalization of microarray L-LPN assay gene expression analysis and gene expression comparison assay results.

TABLE 82
Design Solution Combinations Which Can Be Known to Completely Normalize All, or
Essentially All, Microarray Assay Measured Particular Gene L-LPN Comparison
RASR Values for All Pertinent UNFs and CNFs: Compared Indirectly Labeled
mRNAs Produced from T-RNA

| | | NFs | | Pertinent | |
|---|---|---|---|---|---|
| | | Which Can Be | | NFs To Be | |
| | | Ignored For | | Determined and | |
| | | Normalization | | Normalized For | |
| Combination of Assay Design Solutions | | UNF | CNF | UNF | CNF |
| | Compare Type 1 Labeled mRNA L-LPNs | PAFR | C-HKR | SCR | Spatial |
| | Produced From T-RNAs | LLSR | | MLDR | Print Tip |
| (1) | Combine Table 74 Design Solutions | | | PL-HKR | Print Plate |
| | (a) 1, or 2, or 3, 4a or b, 5a, 6, 8a, c, d, | | | PS-HKR | Intensity |
| | 13b, 14, 22a, 23b, 24b, 26b, 31a-e, 35b, | | | SBNR | Scale |
| | or | | | SSAR | |
| | (b) As (1a), except use Design Solution | | | | |
| | 7 instead of Design Solution 6, or | | | | |
| | (c) As (1a-b), except delete Design | | | | |
| | Solution 8a | | | | |
| (2) | Combine Table 74 Design Solutions | PAFR | — | SCR | C-HKR |
| | (a) As (1a-c), except use Design | LLSR | | MLDR | Spatial |
| | Solution, 13a instead of Design | | | PL-HKR | Print Tip |
| | Solution 13b | | | PS-HKR | Print Plate |
| | | | | SBNR | Intensity |
| | | | | SSAR | Scale |
| | Compare Type 2 Labeled mRNA L-LPNs | PAFR | C-HKR | SCR | Spatial |
| | Produced From T-RNAs | MLDR | | PL-HKR | Print Tip |
| (3) | Combine Table 74 Design Solutions | SBNR | | PS-HKR | Print Plate |
| | (a) 1, or 2, or 3, 4a or b, 5b, 6, 8a, b, | SSAR | | LLSR | Intensity |
| | 13b, 14, 22a, 23b, 24b, 26b, 31b-c, f, | | | | Scale |
| | 35b, or | | | | |
| | (b) As (3a), except use Design Solution | | | | |
| | 7 instead of Design Solution 6 | | | | |
| | (c) As (3a-b), except delete Design | | | | |
| | Solution 8a | | | | |
| (4) | Combine Table 74 Design Solutions | PAFR | — | SCR | C-HKR |
| | (b) As (3a-c), except use Design | MLDR | | PL-HKR | Spatial |
| | Solution, 13a instead of Design | SBNR | | PS-HKR | Print Tip |
| | Solution 13b | SSAR | | LLSR | Print Plate |
| | | | | | Intensity |
| | | | | | Scale |

TABLE 83
Design Solution Combinations Which Can Be Known to Completely Normalize All, or
Essentially All, Microarray Assay Measured Particular Gene L-LPN Comparison
RASR Values for All Pertinent UNFs and CNFs: Compared SG Primed L-LPNs
Produced from T-RNA

| | | NFs | | Pertinent | |
|---|---|---|---|---|---|
| | | Which Can Be | | NFs To Be | |
| | | Ignored For | | Determined and | |
| | | Normalization | | Normalized For | |
| Combination of Assay Design Solutions | | UNF | CNF | UNF | CNF |
| | Comparison of Type 1 L-LPNs | PAFR | C-HKR | SCR | Spatial |
| | Produced From T-RNAs | LLSR | | MLDR | Print Tip |
| | Combine Table 74 Design Solutions | | | PL-HKR | Print Plate |
| | (a) 1, or 2, or 3, 4a or b, 5a, 6, 8a, c, d, | | | PS-HKR | Intensity |
| | 10a, 13b, 14, 15a, 16b, 18b, 31a-e, | | | SBNR | Scale |
| | 35b, or | | | SSAR | |
| | (b) As (1a), except use Design Solution | | | | |
| | 7 instead of Design Solution 6, or | | | | |
| | (c) As (1a-b), except delete Design | | | | |
| | Solution 8a, or | | | | |
| | (d)As (1a-c), except use Design | | | | |
| | Solution 15b instead of Design | | | | |
| | Solution 15a | | | | |
| (1) | Combine Table 74 Design Solutions | PAFR | — | SCR | C-HKR |
| | (a) As (1a-d), except use Design | LLSR | | MLDR | Spatial |
| | Solution, 13a instead of Design | | | PL-HKR | Print Tip |
| | Solution 13b | | | PS-HKR | Print Plate |
| | | | | SBNR | Intensity |
| | | | | SSAR | Scale |
| | Comparison of Type 2 L-LPNs Produced | PAFR | C-HKR | SCR | Spatial |
| | From T-RNAs | MLDR | | PL-HKR | Print Tip |
| (2) | Combine Table 74 Design Solutions | SBNR | | PS-HKR | Print Plate |
| | (a) 1, or 2, or 3, 4a or b, 5b, 6, 8a, b, | SSAR | | LLSR | Intensity |
| | 10a, 13b, 14, 15a, 16b, 18b, 31b-c, | | | | Scale |
| | f, 35b, or | | | | |
| | (b) As (3a-b), except use Design | | | | |
| | Solution 7 instead of Design | | | | |
| | Solution 6, or | | | | |
| | (c) As (3a-b), except delete Design | | | | |
| | Solution 7a, or | | | | |
| | (d) As (3a-c), except use Design | | | | |
| | Solution 15b instead of Design | | | | |
| | Solution 15a | | | | |
| (3) | Combine Table 74 Design Solutions | PAFR | — | SCR | C-HKR |
| | (a) As (3a-d), except use Design | MLDR | | PL-HKR | Spatial |
| | Solution, 13a instead of Design | SBNR | | PS-HKR | Print Tip |
| | Solution 13b | SSAR | | LLSR | Print Plate |
| | | | | | Intensity |
| | | | | | Scale |

TABLE 84
Design Solution Combinations Which Can Be Known to Completely Normalize All, or
Essentially All, Microarray Assay Measured Particular Gene L-LPN Comparison
RASR Values for All Pertinent UNFs and CNFs: Compared Random Primed L-
LPNs Produced from T-RNA

| | | NFs | | Pertinent | |
|---|---|---|---|---|---|
| | | Which Can Be | | NFs To Be | |
| | | Ignored For | | Determined and | |
| | | Normalization | | Normalized For | |
| Combination of Assay Design Solutions | | UNF | CNF | UNF | CNF |
| | Comparison of Type 1 L-LPN Produced | PAFR | C-HKR | SCR | Spatial |
| | From T-RNA | LLSR | | MLDR | Print Tip |
| (1) | Combine Table 74 Design Solutions | | | PL-HKR | Print Plate |
| | (a) 1, or 2, or 3, 4a or b, 5a, 6, 8a, c, d, | | | PS-HKR | Intensity |
| | 13b, 14, 15b, 16b, 18b, 31a-e, 35b | | | SBNR | Scale |
| | or | | | SSAR | |
| | (b) As (1a), except use Design Solution | | | | |
| | 7 instead of Design Solution 6 | | | | |
| | or | | | | |
| | (c) As (1a-b), except delete Design | | | | |
| | Solution 8a | | | | |
| (2) | Combine Table 74 Design Solutions | PAFR | — | SCR | C-HKR |
| | (a) As (1a-c), except use Design | LLSR | | MLDR | Spatial |
| | Solution 13a instead of Design | | | PL-HKR | Print Tip |
| | Solution 13b | | | PS-HKR | Print Plate |
| | | | | SBNR | Intensity |
| | | | | SSAR | Scale |

TABLE 85
Design Solution Combinations Which Can Be Known to Provide Improved Normalization
for All, or Essentially All, Microarray Measured Particular Gene L-LPN Comparison
RASR Values in an Assay for Pertinent UNFs and CNFs: Compared Indirectly
Labeled Oligo dT Primed L-LPNs Produced from T-RNA or Isolated mRNA

| | | NFs | | Pertinent | |
|---|---|---|---|---|---|
| | | Which Can Be | | NFs To Be | |
| | | Ignored For | | Determined and | |
| | | Normalization | | Normalized For | |
| Combination of Assay Design Solutions | | UNF | CNF | UNF | CNF |
| | Compare Type 1 L-LPNs | LLSR | C-HKR | PAFR | Spatial |
| (1) | Combine Table 74 Design Solutions | | | SCR | Print Tip |
| | (a) 1, or 2, or 3, 4a or b, 5a, 6, 8a, c, d, 9a | | | MLDR | Print Plate |
| | or b, 13b, 14, 15a, 16b, 18b, 31a-e, | | | PL-HKR | Intensity |
| | 35b, or | | | PS-HKR | Scale |
| | (b) As (1a), except use Design Solution 7 | | | SBNR | |
| | instead of Design Solution 6, or | | | SSAR | |
| | (c) As (1a-b), except delete Design | | | | |
| | Solution 8a, or | | | | |
| | (d) As (1a-c), except use Design Solution | | | | |
| | 15b instead of Design Solution 15a | | | | |
| (2) | Combine Table 74 Design Solutions | LLSR | — | PAFR | C-HKR |
| | (a) As (1a-d), except use Design Solution, | | | SCR | Spatial |
| | 13a instead of Design Solution 13b | | | MLDR | Print Tip |
| | | | | PL-HKR | Print Plate |
| | | | | PS-HKR | Intensity |
| | | | | SBNR | Scale |
| | | | | SSAR | |
| | Comparison of Type 2 L-LPNs | MLDR | C-HKR | PAFR | Spatial |
| (3) | Combine Table 74 Design Solutions | SBNR | | SCR | Print Tip |
| | (a) 1, or 2, or 3, 4a or b, 5b, 6, 8a, b, 9a, | SSAR | | PL-HKR | Print Plate |
| | b, 13b, 14, 15a, 16b, 18b, 31b, c, f, | | | PS-HKR | Intensity |
| | 35b, or | | | LLSR | Scale |
| | (b) As (3a), except use Design Solution 7 | | | | |
| | instead of Design Solution 6, or | | | | |
| | (c) As (3a-b), except delete Design | | | | |
| | Solution 8a, or | | | | |
| | (d) As (3a-c), except use Design Solution | | | | |
| | 15b instead of Design Solution 15a | | | | |
| (4) | Combine Table 74 Design Solutions | MLDR | — | PAFR | C-HKR |
| | (a) As (3a-d), except use Design Solution, | SBNR | | SCR | Spatial |
| | 13a instead of Design Solution 13b | SSAR | | PL-HKR | Print Tip |
| | | | | PS-HKR | Print Plate |
| | | | | LLSR | Intensity |
| | | | | | Scale |

TABLE 86
Design Solution Combinations Which Can Be Known to Provide Improved Normalization
for All, or Essentially All, Microarray Measured Particular Gene Comparison RASR
Values in an Assay for Pertinent UNFs and CNFs: Compared Indirectly Labeled
mRNA L-LPNs Produced from Isolated mRNAs

| | | NFs | | Pertinent | |
|---|---|---|---|---|---|
| | | Which Can Be | | NFs To Be | |
| | | Ignored For | | Determined and | |
| | | Normalization | | Normalized For | |
| Combination of Assay Design Solutions | | UNF | CNF | UNF | CNF |
| | Compare Type 1 mRNA L-LPNs | LLSR | C-HKR | PAFR | Spatial |
| (1) | Combine Table 74 Design Solutions | | | SCR | Print Tip |
| | (a) 1, or 2, or 3, 4a or b, 5a, 6, 8a, c, d, | | | MLDR | Print Plate |
| | 13b, 14, 22b, 24b, 26b, 31a-e, 35b | | | PL-HKR | Intensity |
| | or | | | PS-HKR | Scale |
| | (b) As (1a), except use Design Solution 7 | | | SBNR | |
| | instead of Design Solution 6 | | | SSAR | |
| | or | | | | |
| | (c) As (1a-b), except delete Design | | | | |
| | Solution 8a | | | | |
| (2) | Combine Table 74 Design Solutions | LLSR | — | PAFR | C-HKR |
| | (a) As (1a-c), except use Design Solution | | | SCR | Spatial |
| | 13a instead of Design Solution 13b | | | MLDR | Print Tip |
| | | | | PL-HKR | Print Plate |
| | | | | PS-HKR | Intensity |
| | | | | SBNR | Scale |
| | | | | SSAR | |
| | Compare Type 2 mRNA L-LPNs | MLDR | C-HKR | PAFR | Spatial |
| (3) | Combine Table 74 Design Solutions | SBNR | | SCR | Print Tip |
| | (a) 1, or 2, or 3, 4a or b, 5b, 6, 8a, b, 13b, | SSAR | | PL-HKR | Print Plate |
| | 14, 22b, | | | PS-HKR | Intensity |
| | 24b, 26b, 31b, c, f, 35b | | | LLSR | Scale |
| | or | | | | |
| | (b) As (3a), except use Design Solution 7 | | | | |
| | instead of | | | | |
| | Design Solution 6 | | | | |
| | or | | | | |
| | (c) As (3a-b), except delete Design | | | | |
| | Solution 8a | | | | |
| (4) | Combine Table 74 Design Solutions | MLDR | — | PAFR | C-HKR |
| | (a) As (3a), except use Design Solution, | SBNR | | SCR | Spatial |
| | 13a instead | SSAR | | PL-HKR | Print Tip |
| | of Design Solution 13b | | | PS-HKR | Print Plate |
| | | | | LLSR | Intensity |
| | | | | | Scale |

TABLE 87
Design Solution Combinations Which Can Be Known to Provide Improved Normalization
for All, or Essentially All, Microarray Measured Particular Gene Comparison RASR
Values in an Assay: Compared Indirectly Labeled SG Primed L-LPNs Produced
from Isolated mRNA

| | | NFs | | Pertinent | |
|---|---|---|---|---|---|
| | | Which Can Be | | NFs To Be | |
| | | Ignored For | | Determined and | |
| | | Normalization | | Normalized For | |
| Combination of Assay Design Solutions | | UNF | CNF | UNF | CNF |
| | Compare Type 1 L-LPNs | LLSR | C-HKR | PAFR | Spatial |
| (1) | Combine Table 74 Design Solutions | | | SCR | Print Tip |
| | (a) 1, or 2, or 3, 4a or b, 5a, 6, 8a, c, d, | | | MLDR | Print Plate |
| | 10b, 13b, 14, 15a, 16b, 18b, 31a-e, 35b, | | | PL-HKR | Intensity |
| | or | | | PS-HKR | Scale |
| | (b) As (1a), except use Design Solution 7 | | | SBNR | |
| | instead of Design Solution 6, or | | | SSAR | |
| | (c) As (1a-b), except delete Design | | | | |
| | Solution 8a, or | | | | |
| | (d) As (1a-c), except use Design Solution | | | | |
| | 15b instead of Design Solution 15a | | | | |
| (2) | Combine Table 74 Design Solutions | LLSR | — | PAFR | C-HKR |
| | (a) As (1a-d), except use Design | | | SCR | Spatial |
| | Solution, 13a instead of Design | | | MLDR | Print Tip |
| | Solution 13b | | | PL-HKR | Print Plate |
| | | | | PS-HKR | Intensity |
| | | | | SBNR | Scale |
| | | | | SSAR | |
| | Compare Type 2 L-LPNs | MLDR | C-HKR | PAFR | Spatial |
| (3) | Combine Table 74 Design Solutions | SBNR | | SCR | Print Tip |
| | (a) 1, or 2, or 3, 4a or b, 5b, 6, 8a, b, 10b, | SSAR | | PL-HKR | Print Plate |
| | 13b, 14, 15a, 16b, 18b, 31b, c, f, 35b, | | | PS-HKR | Intensity |
| | or | | | LLSR | Scale |
| | (b) As (3a), except use Design Solution 7 | | | | |
| | instead of Design Solution 6, or | | | | |
| | (c) As (3a-b), except delete Design | | | | |
| | Solution 8a, or | | | | |
| | (d) As (3a-c), except use Design Solution | | | | |
| | 15b instead of Design Solution 15a | | | | |
| (4) | Combine Table 74 Design Solutions | MLDR | — | PAFR | C-HKR |
| | (a) As (3a-d), except use Design Solution, | SBNR | | SCR | Spatial |
| | 13a instead of Design Solution 13b | SSAR | | PL-HKR | Print Tip |
| | | | | PS-HKR | Print Plate |
| | | | | LLSR | Intensity |
| | | | | | Scale |

TABLE 88
Design Solution Combinations Which Can Be Known to Provide Improved
Normalization for All, or Essentially All, Microarray Measured Particular
Gene Comparison RASR Values in an Assay: Compared Indirectly Random Primed
L-LPNs Produced from Isolated mRNAs

| | | NFs | | Pertinent | |
|---|---|---|---|---|---|
| | | Which Can Be | | NFs To Be | |
| | | Ignored For | | Determined and | |
| | | Normalization | | Normalized For | |
| Combination of Assay Design Solutions | | UNF | CNF | UNF | CNF |
| | Compare of Type 1 L-LPNs | LLSR | C-HKR | PAFR | Spatial |
| (1) | Combine Table 74 Design Solutions | | | SCR | Print Tip |
| | (a) 1, or 2, or 3, 4a or b, 5a, 6, 8a, c, d, | | | MLDR | Print Plate |
| | 13b, 14, 15b, 16b, 18b, 31a-e, 35b | | | PL-HKR | Intensity |
| | or | | | PS-HKR | Scale |
| | (b) As (1a), except use Design Solution | | | SBNR | |
| | 7 instead of Design Solution 6 | | | SSAR | |
| | or | | | | |
| | (c) As (1a-b), except delete Design | | | | |
| | Solution 8a | | | | |
| (2) | Combine Table 74 Design Solutions | LLSR | — | PAFR | C-HKR |
| | (b) As (1a-c), except use Design | | | SCR | Spatial |
| | Solution 13a instead | | | MLDR | Print Tip |
| | of Design Solution 13b | | | PL-HKR | Print Plate |
| | | | | PS-HKR | Intensity |
| | | | | SBNR | Scale |
| | | | | SSAR | |

TABLE 89
Design Solution Combinations Which Can Be Known to Provide Improved Normalization
for All, or Essentially All, Particular Gene Comparison RASR Values in an Assay,
and More Complete Normalization for an Identifiable Subset of Particular Gene
Comparison RASR Values in the Assay

| | | NFs Which Can Be | | | | |
|---|---|---|---|---|---|---|
| | | Ignored For | | Pertinent NFs To Be Determined | | |
| | | Normalization | | and Normalized For | | |
| | | More | Rest of | More | | |
| | | Completely | Particular | Completely | Rest of | |
| Combination of Assay | | Normalized | Gene | Normalized | Particular Gene | |
| Design Solutions | | Subset | Comparisons | Subset | Comparison | |
| | Compare Type 1 Oligo | MLDR* | LLSR | PAFR | PAFR | Spatial |
| | dT Primed L-LPNs | PL-HKR* | | SCR | SCR | Print Tip |
| (1) | Combine Table 74 Design | PS-HKR* | | SSAR | SSAR | Print Plate |
| | Solutions | SBNR* | | Spatial | MLDR | Intensity |
| | (a) 1, or 2, or 3, 4a or b, 5a, | LLSR | | Print Tip | PL-HKR | Scale |
| | 6, 8a, c, d, 9a or b, 13a, | | | Print Plate | PS-HKR | C-HKR |
| | 14, 15b, 16b, 20, 32, 35a | | | Intensity | SBNR | |
| | | | | Scale | | |
| | | | | C-HKR | | |
| (2) | Combine Table 74 Design | MLDR* | LLSR | PAFR | PAFR | Spatial |
| | Solutions | PL-HKR* | C-HKR | SCR | SCR | Print Tip |
| | (a) As (1a), except use | PS-HKR* | | SBNR | SSAR | Print Plate |
| | Design Solution 13b | LLSR | | SSAR | MLDR | Intensity |
| | instead of Design | C-HKR* | | Spatial | PL-HKR | Scale |
| | Solution 13a | | | Print Tip | PS-HKR | |
| | | | | Print Plate | SBNR | |
| | | | | Intensity | | |
| | | | | Scale | | |
| | Compare Oligo dT Primed | MLDR | MLDR | C-HKR | PAFR | Spatial |
| | Type 2 L-LPNs | PL-HKR* | SBNR | PAFR | SCR | Print Tip |
| (3) | Combine Table 74 Design | PS-HKR* | SSAR | SCR | LLSR | Print Plate |
| | Solutions | SBNR | | LLSR | PL-HKR | Intensity |
| | (a) 1, or 2, or 3, 4a or b, 5b, | SSAR | | Spatial | PS-HKR | Scale |
| | 6, 8a, b, 9a or b, 13a, 14, 15b, | | | Print Tip | | |
| | 16b, 20, 32, 35a | | | Print Plate | | |
| | | | | Intensity | | |
| | | | | Scale | | |
| (4) | Combine Table 74 Design | MLDR | MLDR | PAFR | PAFR | Spatial |
| | Solutions | PL-HKR* | SBNR | SCR | SCR | Print Tip |
| | (a) As (3a), except use | PS-HKR* | SSAR | LLSR | LLSR | Print Plate |
| | Design Solution 13b | SBNR | C-HKR* | Spatial | PL-HKR | Intensity |
| | instead of Design | SSAR | | Print Tip | PS-HKR | Scale |
| | Solution 13a | C-HKR* | | Print Plate | | |
| | | | | Intensity | | |
| | | | | Scale | | |

*Assay value is equal to one.

TABLE 90
Design Solution Combinations Which Can Be Known to Minimize or Eliminate the
Occurrence of Microarray Assay Generated UNF and CNF Related Particular Gene
False Negative Results and Associated RDMs

| | | | | Pertinent NFs To Be | |
|---|---|---|---|---|---|
| | | NFs Which Can | | Determined and | |
| | | Be Ignored For Normalization | | Normalized For | |
| Combination of Assay Design Solutions | | UNF | CNF | UNF | CNF |
| (1) | Combine Table 74 Design Solutions | Any Pertinent | Any Pertinent | The Rest | The Rest |
| | (a) As described in Tables 75-89, except also | UNF = 1 | CNF = 1 | | |
| | use Design Solution 34 | | | | |

The known design solution combination associated with a microarray assay comparison of cell sample L-LPNs determines whether the assay can be known to be associated with improved normalization of assay measured particular gene RASR values, and the degree to which the normalization can be known to be improved, relative to prior art L-LPN microarray normalization practice. As discussed, prior art microarray L-LPN practice does not determine and normalize for pertinent UNFs, and in addition the key assumptions necessary for the valid prior art normalization of pertinent CNFs, are known to be invalid for certain prior art L-LPN microarray assays, and cannot be known to be valid for the large majority of, if not all, prior art L-LPN microarray assays. Prior art L-LPN microarray practice does not provide the information necessary for determining the design solution combination associated with a particular prior art microarray indirect label L-LPN comparison assay. These factors create a situation where the design solution combination associated with any particular prior art microarray assay is not known. This means that, except for those prior art L-LPN microarray assays which are known to be invalidly normalized for certain CNFs, and/or not normalized for certain UNFs, the completeness and validity of normalization for other prior art L-LPN microarray assay results cannot be known. The prior art produced particular gene comparison NASR values for these assays are then, uninterpretable. It is possible, but not likely, that unknown to prior art L-LPN microarray practice, a particular prior art L-LPN microarray assay is associated with incomplete but improved normalization. Absent knowledge of the design solution combination associated with the prior art assay however, it cannot be known whether the assay is associated with improved normalization or not.

The design solution combination associated with an L-LPN microarray assay determines the following. (i) the validity of the pertinent CNF normalization. (ii) the completeness of normalization for pertinent UNFs and CNFs. (iii) the fraction of particular gene comparison RASR values in the assay which can be maximally normalized for pertinent UNFs and CNFs. (iv) the ease of determining the assay values for pertinent CNFs and UNFs. (v) the ease and simplicity of the normalization process. (vi) the biological accuracy of the normalized particular gene NASR values for an assay. (vii) the overall interpretability of the normalized particular gene comparison NASR values. (viii) the between and within assay intercomparability of the normalized particular gene comparison NASR values. (ix) the intercomparability of an L-LPN microarray measured cell sample particular gene N-DGER value with a cell sample particular gene N-DGER value obtained with a different microarray or non-microarray assay method, for which the design solution combination associated with the assay is known. Here, if the L-LPN microarray assay measured particular gene N-DGER value is biologically accurate, then; the normalization is valid and complete; the particular gene N-DGER value can be validly interpreted as to quantitative extent of gene expression difference and direction of regulation change; the particular gene N-DGER value can be validly intercompared with other biologically accurate microarray or non-microarray particular gene N-DGER values which have been obtained with other direct or indirect labeled microarray or non-microarray methods. It is desirable to maximize each of the above noted characteristics as much as possible. Tables 75-90 present samples of such a maximization effort. It will be useful to discuss certain of these examples in more detail.

The majority of prior art cell sample L-LPN comparisons involve the direct comparison of cell sample oligo dT primed cDNA L-LPN, or the use of oligo dT primed cell sample cDNA to produce cell sample L-LPN cRNAs which are compared. In addition, most of these oligo dT primed L-LPN associated assays involve the use of just one ligand to label each compared L-LPN prep. The design solution combination presented in Table 78(7), represents such an oligo dT primed L-LPN, one ligand assay. This design solution combination provides improved normalization for all particular gene RASR values in the assay for all of the pertinent UNFs and CNFs, except PAFR. The aspects of improved normalization associated with most of the design solutions used for Table 78(7) were discussed earlier in the section on direct labeled LPN microarray normalization improvement. Again, Design Solution is termed DS. For Table 78(7) DS (16a) and (18a) ensure that the compared particular gene L-LPN molecules are the same nucleotide length, DS (35a) ensures that the compared particular gene L-LPN ligand densities are similar, and DS (13a) ensures that identical SGC molecules are used to stain each compared array. This design solution combination ensures that the SBNR assay value for each particular gene L-LPN comparison in the assay is equal to one or nearly one. Further, as discussed earlier, DS (16a) and DS (18a) ensure that the MLDR, PL-HKR, and PS-HKR assay values for all particular gene L-LPN comparisons in the assay are equal to one or nearly one. It is likely that for a carefully done assay, the DS (16a), (18a), (35a) and (13a) combination, also ensures that the SSAR value for all particular gene L-LPN comparisons is also equal to one, or nearly one. This needs to be confirmed. As a consequence of the Table 48(7) design solution combination, all of the particular gene L-LPN comparisons can undergo improved normalization for all pertinent UNFs and CNFs, except the PAFR. Further, because of the improved assay design, the only assay UNF values which must be determined are the SCR which is equal to one (DS30a), and possibly the SSAR. In addition, the use of DS (6) provides for the improved normalization of the pertinent assay CNFs. As indicated in Tables 75 through 90, a variety of different design solution permutations provide improved normalization process and improved normalization particular gene RASR values.

The preferred and other design solution combinations described in Table 75 through 90 represent only a fraction of the possible design solution combinations which can provide improved normalization and results. Table 89 describes design solution combinations which provide different degrees of improvement for different identifiable subsets of particular gene RASR values in the assay. This situation was discussed earlier in the section on directly labeled LPNs.

Table 90 presents L-LPN assay design solution combinations which can be known to minimize or eliminate the occurrence of UNF and CNF related particular gene false negative results and associated RDMs. This was also discussed earlier.

A microarray L-LPN assay can be described by the design solution combination which is associated with the assay. An accurate assay design solution combination description serves as the basis for identifying the following. (i) The pertinent UNFs and CNFs which are associated with the assay. (ii) The pertinent UNFs and CNFs which can be ignored during the assay normalization process. (iii) The pertinent UNF and CNF assay values which must be determined and normalized for in the assay. (iv) The pertinent UNFs and CNFs which can be determined and normalized for. (v) The pertinent UNFs and CNFs which are normalized for. (vi) The assumptions necessary to determine UNF and CNF assay values. Such an overall description is necessary in order to evaluate the utility, biological accuracy, reproducibility, and intercomparability of the assay measured particular gene comparison NASR values. Such an overall description should be available for every microarray L-LPN assay. Such an overall design solution combination description can be used to plan future microarray L-LPN assays, and to interpret already existing microarray L-LPN assay particular gene comparison normalized results or NASR values. Such overall design solution combination descriptions were not created for prior art L-LPN microarray assays of any kind. In addition, such an overall design solution combination description will allow the effective standardization of microarray assay formats.

Improvement of Non-Microarray Northern Blot, Dot Blot and Nuclease Protection Assay Normalization Process.

The northern blot, dot blot and nuclease protection assays are widely used prior art gene expression analysis and comparison methods. Such methods are often used by the prior art to validate prior art microarray measured and normalized particular gene NASR values. Of these, the northern blot method is, by far, the most frequently used for this purpose. In contrast to microarrays, these non-microarray assays are generally used to determine the expression of only one or several, particular genes.

These prior art non-microarray assays are also associated with pertinent UNFs and CNFs. However, relative to microarray assays, these non-microarray assays are associated with a smaller number of pertinent UNFs and CNFs. Prior art non-microarray assay design is not standardized, and there are a variety of different assay designs which are commonly used for each particular non-microarray method. Table 91 identifies the pertinent UNFs and CNFs which are associated with these various non-microarray methods which are common for each method. Each of the alternative assay situations described in Table 91 represents a prior art non-microarray practice situation, and the pertinent UNFs and CNFs which must be accurately determined and normalized for during the normalization process, in order to obtain improved non-microarray measured assay results.

TABLE 91
UNFs and CNFs Associated with Prior Art Northern Blot,
Dot Blot, and Nuclease Protection Assays

| | UNFs and CNFs Which May Be Pertinent | | | |
|---|---|---|---|---|
| | When Comparing Cell Sample | | | |
| | | | Undegraded | Degraded |
| Non-Microarray | Undegraded | Degraded | Isolated | Isolated |
| Method | T-RNAs | T-RNAs | mRNAs | mRNAs |
| Northern Blot | SCR | — | SCR | — |
| | C-HKR | | PAFR | |
| | | | C-HKR | |
| Dot Blot | SCR | SCR | SCR | SCR |
| | C-HKR | MLDR | PAFR | PAFR |
| | | C-HKR | C-HKR | MLDR |
| | | | | C-HKR |
| Nuclease Protection | SCR | SCR | SCR | SCR |
| | C-HKR | C-HKR | PAFR | PAFR |
| | | | C-HKR | MLDR |
| | | | | C-HKR |

The determination of the assay values for the UNFs, and their use for normalization were discussed earlier. A large majority of prior art non-microarray assays analyze the expression of only one or a few particular genes. This greatly simplifies the determination of certain pertinent UNF assay values. In this situation it is practical, albeit labor intensive and complex, to determine the assay PAFR value for a particular gene. The assay values for SCR and MLDR can also be determined, as described earlier. Note that for dot blot and northern blot assays, the assay variables associated with efficiency of RNA immobilization, and the hybridization availabilities and efficiencies of the immobilized RNA, are reflected in the assay SCR value. In addition, the RNA electrophoretic efficiency for northern blots is also reflected in the assay SCR value. Northern blot analysis is not recommended for degraded cell sample T-RNA or isolated mRNA. An advantage of the nuclease protection method over the northern blot and dot blot methods, is the absence of these immobilization and electrophoresis associated assay variables. Most northern blot, dot blot, and nuclease protection assays utilize radioactive labels.

The large majority of northern blot, dot blot and nuclease protection assays involve the use of a directly labeled particular gene LPN. The UNFs associated with these direct label assays are presented in Table 91. These non-microarray assays seldom involve indirectly labeled L-LPNs. These L-LPN associated assays would be associated with the UNFs listed in Table 91, as well as the UNFs SBNR and SSAR, which are discussed extensively in the earlier sections on microarray L-LPN assays and on the improvement of normalization of microarray L-LPN assay results. The discussions in these sections relating to the SBNR and SAR UNFs apply directly to northern blot, dot blot and nuclease protection assays involving L-LPNs.

In order to obtain non-microarray assay measured improved particular gene NASR and N-DGER values which are biologically accurate, it is necessary to use an improved overall process for the complete and accurate normalization of the non-microarray assay measured particular gene results. This includes the identification of the pertinent UNFs and CNFs for each assay, and the accurate determination for the assay values for the pertinent UNFs and CNFs, as well as the accurate normalization for the pertinent UNFs and CNFs.

Prior art non-microarray gene expression analysis and comparison practice does not determine the assay value for, or normalize particular gene comparison RASR values for pertinent UNFs. Each pertinent UNF can cause an assay measured particular gene comparison result value to deviate significantly from biological accuracy when the UNF value deviates significantly from one. Table 51 presents the previously discussed estimates of the magnitudes of the deviation from one which are believed to commonly occur for the UNFs of prior art microarray assays, as well as the commonly claimed measurement accuracies for prior art microarray assays. These Table 51 estimated UNF values are also believed to commonly occur for prior art non-microarray assays. In addition, assay measurement accuracies of about ±1.2 have been claimed for nuclease protection assay comparison particular gene NASR values. Northern blot and dot blot assays are generally considered to be less accurate. It is likely that most prior art non-microarray assays are associated with at least one UNF which does not equal one, and many are likely to be associated with more than one such UNF value. In the context of the measurement accuracy of a non-microarray northern blot, dot blot, or nuclease protection assay, the deviation of the pertinent UNFs SCR or MLDR from one is enough to significantly affect the quantitative value land interpretation of a prior art measured particular gene result value. In this same context, the deviation of the pertinent UNF PAFR from one is enough to significantly affect the quantitative value and interpretation of a nuclease protection assay measured particular gene RASR value. Further, prior art non-microarray practice does not determine the assay values for the UNFs, and as a result it cannot be known whether a prior art non-microarray measured particular gene comparison RASR value requires normalization for pertinent UNFs or not. Therefore, it is necessary to first identify each UNF which is pertinent for a non-microarray assay, and then to determine a measure of the pertinent UNF assay value in order to determine whether UNF normalization is necessary, and then to normalize the particular gene RASR value for the UNF values, if UNF normalization is required. For a typical non-microarray assay the requirement to determine and normalize for the pertinent UNFs adds a significant amount of complexity and effort to the non-microarray assay, relative to the prior art non-microarray assay. In addition, systematic measurement error and noise will be associated with experimentally determined UNF values, and their use for normalization.

These considerations make it very desirable, if not necessary, to simplify the determination of pertinent UNF assay values and the normalization process as much as possible, and to eliminate the need to experimentally determine assay values for as many UNFs as possible. Here it is particularly desirable to eliminate the need to experimentally determine the assay values for those UNFs which are difficult or labor intensive to determine, such as the PAFR.

Earlier sections extensively discussed the underlying basis for each assay UNF, and the assay situations under which each UNF is pertinent. As a result of this, it is possible to identify the assay factors which can and must be controlled for different assay situations, in order to simplify the process for determining the pertinent UNF assay values and normalizing for them. This knowledge makes it possible to design non-microarray assays which do not require the direct determination of certain pertinent UNFs in order to know that such UNFs are validly normalized for. The overall result of such assay designs is a simplified version of the improved non-microarray normalization process. This can be accomplished by judicious assay design and measurement, as is discussed below.

The various design approaches which will result in an improved normalization process relative to prior art normalization processes, are presented in Table 52. The successful implementation of any one of the Table 52 design approaches 1-8 will produce a normalization process which can be known to be improved, relative to prior art non-microarray normalization practices. The successful implementation of Table 52 design approach 9, will produce non-microarray assay results which are known to contain fewer NF related false negative results than prior art microarray results. Prior art non-microarray assay design is not standardized and there are a variety of different assay designs which are commonly used to reach particular non-microarray method. The improvement of the normalization process for each of these alternate assay designs will be discussed. Design components or design solutions which can be used to produce improved non-microarray assay normalization are presented in Table 92. Each of these design solutions or design components reflects an aspect of non-microarray assay design which directly or indirectly impacts on an assay pertinent UNF or CNF. Different combinations of these design solutions can be used to describe an overall non-microarray assay which is improved, relative to a prior art RT-PCR assay.

TABLE 92
Design Solutions for Improving the Prior Art Non-Microarray Northern Blot, Dot Blot,
Nuclease Protection and RT-PCR Assay Normalization Process and the Assay
Measured Particular Gene NASR Values

| | | NFs Which | | NFs Which | |
|---|---|---|---|---|---|
| | | May Be | | Can Be | |
| | | Pertinent to | | Ignored For | |
| | | Assay | | Normalization | |
| Non-Microarray Assay Design Solution | | UNFs | CNFs | UNFs | CNFs |
| (1) | Use the Northern Blot method to | SCR | C-HKR | — | — |
| | (a) Assay for one particular gene mRNA | PAFR | | | |
| | (b) Assay for multiple particular gene mRNAs | MLDR | | | |
| (2) | Use the Dot Blot method to | SCR | C-HKR | — | — |
| | (a) Assay for one particular gene mRNA | PAFR | | | |
| | (b) Assay for multiple particular gene mRNAs | MLDR | | | |
| (3) | Use the Nuclease Protection method to | SCR | C-HKR | — | — |
| | (a) Assay for one particular gene mRNA | PAFR | | | |
| | (b) Assay for multiple particular gene mRNAs | MLDR | | | |
| (4) | Use the RT-PCR method to | SCR | PG AE · SER | — | — |
| | (a) Assay for one particular gene mRNA | PAFR | S AE · SER | | |
| | (b) Assay for multiple particular gene mRNAs | | PG AE · AER | | |
| | | | S AE · AER | | |
| (5) | Use (a) Radioactive label | SCR | C-HKR | — | — |
| | (b) Non-radioactive label | PAFR | | | |
| | | MLDR | | | |
| (6) | Use (a) One label for a particular gene LPN | SCR | C-HKR | — | — |
| | | PAFR | | | |
| | | MLDR | | | |
| | (b) Different labels for different particular | SCR | C-HKR | — | — |
| | gene mRNAs in assay | PAFR | | | |
| | | MLDR | | | |
| (7) | Use (a) One oligonucleotide LPN | SCR | C-HKR | — | — |
| | | PAFR | | | |
| | (b) Multiple oligonucleotide LPNs in an | SCR | C-HKR | — | — |
| | assay for one particular gene mRNA | PAFR | | | |
| | | MLDR | | | |
| (8) | Use (a) Undegraded non-oligonucleotide LPN | SCR | C-HKR | — | — |
| | molecules | PAFR | | | |
| | (b) Non-oligonucleotide LPN which | SCR | C-HKR | — | — |
| | consists of multiple LPN fragments which are | PAFR | | | |
| | complementary to a single particular gene | MLDR | | | |
| | undegraded mRNA molecule | | | | |
| (9) | Use (a) An RNA LPN | SCR | — | — | — |
| | | PAFR | | | |
| | | MLDR | | | |
| | (b) A DNA LPN for the assay | SCR | C-HKR | — | — |
| | | PAFR | | | |
| | | MLDR | | | |
| (10) | Use a particular gene LPN which is | | | | |
| | complementary to | | | | |
| | (a) The 3′ end portion | SCR | C-HKR | — | — |
| | | PAFR | | | |
| | | MLDR | | | |
| | (b) The 5′ end portion | SCR | C-HKR | — | — |
| | | PAFR | | | |
| | | MLDR | | | |
| | (c) Both the 3′ end and 5′ end portion of the | SCR | C-HKR | — | — |
| | particular gene mRNA | PAFR | | | |
| | | MLDR | | | |
| (11) | Use (a) Type 1 LPN | SCR | C-HKR | — | — |
| | | PAFR | | | |
| | | MLDR | | | |
| | (b) Type 2 LPN | SCR | C-HKR | MLDR | — |
| | | PAFR | | | |
| (12) | Use (a) A single strand LPN of one polarity | SCR | C-HKR | — | — |
| | | PAFR | | | |
| | | MLDR | | | |
| | (b) A denatured double strand LPN | SCR | C-HKR | — | — |
| | | PAFR | | | |
| | | MLDR | | | |
| (13) | Use (a) One hybridization solution | SCR | C-HKR | — | C-HKR |
| | | PAFR | | | |
| | | MLDR | | | |
| | (b) Two hybridization solutions for the | SCR | C-HKR | — | — |
| | assay | PAFR | | | |
| | | MLDR | | | |
| (14) | Use hybridization conditions which ensure that | SCR | C-HKR | — | C-HKR |
| | the LPN hybridization to the RNA goes to | PAFR | | | |
| | completion | MLDR | | | |
| (15) | Compare cell sample | | | | |
| | (a) T-RNA | SCR | C-HKR | PAFR | — |
| | | MLDR | | | |
| | (b) Isolated mRNA | SCR | C-HKR | — | — |
| | | PAFR | | | |
| | | MLDR | | | |
| (16) | The average nucleotide lengths of the | | | | |
| | compared T-RNA preps are | | | | |
| | (a) The same | SCR | C-HKR | PAFR | — |
| | | | | MLDR | |
| | (b) Different | SCR | C-HKR | PAFR | — |
| | | MLDR | | | |
| (17) | The average nucleotide lengths of the | | | | |
| | compared Isolated mRNA preps are | | | | |
| | (a) The same | SCR | C-HKR | MLDR | — |
| | | PAFR | | | |
| | (b) Different | SCR | C-HKR | — | — |
| | | PAFR | | | |
| | | MLDR | | | |
| (18) | The nucleotide lengths and nucleotide | | | | |
| | sequences for compared particular mRNAs | | | | |
| | are | | | | |
| | (a) The same | SCR | C-HKR | MLDR | — |
| | | PAFR | | | |
| | (b) Different | SCR | C-HKR | — | — |
| | | PAFR | | | |
| | | MLDR | | | |
| (19) | The nucleotide lengths and nucleotide | SCR | C-HKR | MLDR | — |
| | sequences are the same for all compared | PAFR | | | |
| | particular gene mRNA molecules in the assay | | | | |
| (20) | The nucleotide lengths and nucleotide | SCR | C-HKR | — | — |
| | sequences are the same for less than all compared | PAFR | | | |
| | particular gene mRNA molecules in the assay | MLDR | | | |
| (21) | Compare particular gene undegraded mRNAs | SCR | C-HKR | — | — |
| | | PAFR | | | |
| | | MLDR | | | |
| (22) | For Northern Blot, compared particular gene | SCR | C-HKR | — | — |
| | mRNA molecules have the same nucleotide lengths | PAFR | | | |
| | and nucleotide sequences, and have the same | MLDR | | | |
| | electrophoretic, surface immobilization, and | | | | |
| | hybridization availability and kinetic efficiencies | | | | |
| | and characteristics | | | | |
| (23) | For Dot Blot, compared particular gene mRNA | SCR | C-HKR | — | — |
| | molecules of the same, or different nucleotide | PAFR | | | |
| | lengths and nucleotide sequences, have the same | MLDR | | | |
| | surface immobilization and hybridization | | | | |
| | availability and kinetic efficiencies and | | | | |
| | characteristics | | | | |
| (24) | For Dot Blot or Northern Blot assays, nuclease | SCR | C-HKR | — | — |
| | treat after hybridization | PAFR | | | |
| | | MLDR | | | |
| (25) | Maximize the number of UNFs and CNFs | SCR | C-HKR | — | — |
| | which have an assay value equal to one | PAFR | | | |
| | | MLDR | | | |
| (26) | Use AHG and/or other standards to determine | SCR | C-HKR | — | — |
| | and normalize for | PAFR | | | |
| | (a) C-HKR | MLDR | | | |
| | (b) SCR | | | | |
| | The following Design Solutions apply only to RT- | | | | |
| | PCR Assays | | | | |
| (27) | Use (a) SG Primer | SCR | PG AE · SER | — | — |
| | (b) Oligo dT Primer | PAFR | S AE · SER | | |
| | (c) Random Primer | | PG AE · AER | | |
| | to produce cDNA | | S AE · AER | | |
| (29) (28) | Use SG primers targeted to the extreme 3′ | SCR | PG AE · SER | — | — |
| | end of the particular gene mRNA or assay | PAFR | S AE · SER | | |
| | standard mRNA | | PG AE · AER | | |
| | | | S AE · AER | | |
| (30) | RT synthesized cDNA nucleotide lengths for a | SCR | PG AE · SER | — | — |
| | cell sample particular gene or assay standard | PAFR | S AE · SER | | |
| | cDNA are as short as possible | | PG AE · AER | | |
| | | | S AE · AER | | |
| (31) | RT synthesized cDNA nucleotide lengths for a | SCR | PG AE · SER | — | — |
| | cell sample particular gene cDNA or assay | PAFR | S AE · SER | | |
| | standard cDNA are the same or essentially the | | PG AE · AER | | |
| | same | | S AE · AER | | |
| (32) | RT synthesized cDNA nucleotide lengths for | SCR | PG AE · SER | — | — |
| | compared cell sample particular gene cDNAs | PAFR | S AE · SER | | |
| | or compared assay standard cDNAs are the | | PG AE · AER | | |
| | same or essentially the same | | S AE · AER | | |
| (33) | The nucleotide sequences of the compared cell | SCR | PG AE · SER | — | — |
| | sample particular gene synthesized cDNAs or | PAFR | S AE · SER | | |
| | compared assay standard synthesized cDNAs | | PG AE · AER | | |
| | are the same or essentially the same | | S AE · AER | | |
| (34) | For each separate assay determine the cell | SCR | PG AE · SER | — | — |
| | sample's particular gene AE · SE assay value, | PAFR | S AE · SER | | |
| | and use said AE · SE value to normalize the | | PG AE · AER | | |
| | said particular gene assay result | | S AE · AER | | |
| (35) | Predetermine the AE · SE values for a | SCR | PG AE · SER | — | — |
| | particular gene and standard combination, and | PAFR | S AE · SER | | |
| | use the predetermined values for | | PG AE · AER | | |
| | normalization of particular gene assay results | | S AE · AER | | |
| | from other assays without determining the | | | | |
| | particular gene and standard AE · SE assay | | | | |
| | value for each assay | | | | |
| (36) | For each separate assay, determine the | SCR | PG AE · SER | — | — |
| | compared cell sample's particular gene and | PAFR | S AE · SER | | |
| | assay standard AE · SE assay values and use | | PG AE · AER | | |
| | said values to normalize said separate assay's | | S AE · AER | | |
| | particular gene results. | | | | |
| (37) | Make each particular gene or standard PCR | SCR | PG AE · SER | — | — |
| | amplicon nucleotide length as short as | PAFR | S AE · SER | | |
| | possible | | PG AE · AER | | |
| | | | S AE · AER | | |
| (37) | Design each particular gene or standard PCR | SCR | PG AE · SER | — | — |
| | amplicon to be as close to the mRNA 3′ end | PAFR | S AE · SER | | |
| | as possible | | PG AE · AER | | |
| | | | S AE · AER | | |
| (38) | Use highly purified PCR primers for the assay | SCR | PG AE · SER | — | — |
| | | PAFR | S AE · SER | | |
| | | | PG AE · AER | | |
| | | | S AE · AER | | |
| (39) | Design the PCR amplicon primer | SCR | PG AE · SER | — | — |
| | combinations so that for an assay the | PAFR | S AE · SER | | |
| | particular gene and standard amplification | | PG AE · AER | | |
| | efficiencies are the same or very similar | | S AE · AER | | |
| (40) | For each separate assay determine the cell | SCR | PG AE · SER | — | — |
| | sample's particular gene AE · AE value, and | PAFR | S AE · SER | | |
| | use said AE · AE value to normalize said | | PG AE · AER | | |
| | particular gene assay result | | S AE · AER | | |
| (41) | Predetermine the AE · AE values for a | SCR | PG AE · SER | — | — |
| | particular gene and standard combination, and | PAFR | S AE · SER | | |
| | use the predetermined values for | | PG AE · AER | | |
| | normalization of particular gene assay results | | S AE · AER | | |
| | from other assays without determining the | | | | |
| | particular gene and standard AE · AE assay | | | | |
| | value for each assay. | | | | |
| (42) | For each separate assay, determine the | SCR | PG AE · SER | — | — |
| | compared cell sample's particular gene and | PAFR | S AE · SER | | |
| | assay standard AE · AE assay values, and use | | PG AE · AER | | |
| | said values to normalize said separate assays | | S AE · AER | | |
| | particular gene results | | | | |

Certain of these design solutions are discussed and further defined below. Here, design solution will be termed DS.

DS 1, 2, 3.

The great majority of the prior art assays done with these methods, assay only one particular gene's mRNA per assay. Prior art considers the northern blot and dot blot methods to be less accurate than the nuclease protection and RT-PCR methods. DS 5, 6. The great majority of non-microarray northern blot, dot blot, and nuclease protection assays utilize radioactive labels and use one label for each particular gene comparison. As discussed earlier, the label used may be an indirect or direct label. DS 7, 8, 9. Both RNA and DNA oligonucleotide and non-oligonucleotide LPNs are used for northern blot, dot blot, and nuclease protection assays.

DS10.

For northern blot, dot blot, and nuclease protection assays, all three approaches have been used. Overall, the most versatile approach is DS10a.

DS 11.

Both Type 1 and Type 2 LPNs are used for northern blot, dot blot and nuclease protection assays.

DS 12.

Single strand LPN of one polarity is generally necessary for nuclease protection assays, and is preferred for northern blot and dot blots.

DS 13.

Northern blot and dot blot cell sample comparison assays almost always employ only one hybridization solution, while nuclease protection assays require two hybridization solutions.

DS 14.

All well designed northern blot, dot blot, and nuclease protection assays are designed to ensure hybridization to completion.

DS 15.

Non-microarray assays commonly compare either T-RNAs or isolated mRNAs.

DS 16, 17.

The overall cell sample prep RNA molecule population average nucleotide length reflects a complex average of all of the different RNA molecule populations which are present in the RNA prep. Compared cell sample RNA molecule populations often have different average nucleotide lengths.

DS 18, 19, 20, 21.

Here the same nucleotide lengths and nucleotide sequences refers to one of the following situations. (i) The compared particular gene mRNAs are undegraded and the nucleotide lengths and nucleotide sequences of the compared RNA molecules are identical. (ii) The compared particular gene mRNA molecule populations are degraded and have the same average nucleotide lengths and nucleotide sequence distributions, and therefore represent the same particular gene nucleotide sequence. Valid northern blot assays require (i), while dot blot and nuclease protection assays are effective with either (i) or (ii). Non-microarray gene expression analysis and comparison assays often compare degraded RNAs.

DS 22, 23.

Northern blot and dot blot assays are generally considered to be less accurate than nuclease protection or RT-PCR assays. This results largely from the lack of information concerning the assay characteristics described.

DS 24.

Post-hybridization nuclease treatment is not routinely used for northern blot and dot blot assays. Such treatment can control for differences in size between the immobilized RNA and the labeled LPN.

DS 25.

This will minimize or eliminate the occurrence of UNF and CNF related false negative results and their associated RDMs.

DS 26.

This is most useful for nuclease protection assays but should not be needed for a properly designed assay.

Relative to prior art normalization practice, the normalization of non-microarray measured particular gene analysis and comparison results is improved when one or more particular gene RAS or RASR values produced by a non-microarray assay is known to be validly normalized for one or more of the following. (i) one or more pertinent UNFs. (ii) one or more pertinent UNFs and one or more pertinent CNFs. (iii) one or more pertinent UNFs and all pertinent CNFs. (iv) all pertinent UNFs and all pertinent CNFs. For a northern blot, dot blot, or nuclease protection assay the preferred improved normalization process assay design results in the valid normalization of all particular gene RAS or RASR values in an assay, for all pertinent UNFs and CNFs, and also results in minimizing the number of UNF and CNF related false negative results which are associated with the assay. Such assay designs are described below. A variety of different non-microarray assay formats are practiced by the prior art, and different formats can be associated with different combinations of pertinent UNFs and CNFs. For simplicity each different prior art general assay design will be discussed in terms of the Table 92 design solutions combinations which can be known to allow the improved normalization of all or essentially all particular gene RAS or RASR values in the assay for the maximum number of pertinent UNFs and CNFs. These preferred practice design solution combinations, and other design solution combinations which also can be known to produce improved normalization and assay results, are presented in Tables 93 through 95. Note that the design solution combinations presented represent only a few of the many possible different design solution combinations which will produce an improved non-microarray assay normalization process and improved assay results.

TABLE 93
Preferred and Other Practices for Design Solution Combinations Which Can Be Known
to Provide Improved Normalization of Northern Blot Particular Gene RASR Values for
All, or One or More, Pertinent UNFs and/or CNFs

| | | NFs Which Can Be | | Pertinent NFs To Be | |
|---|---|---|---|---|---|
| | | Ignored For | | Determined and | |
| | | Normalization | | Normalized For | |
| Combination of Assay Design Solutions | | UNF | CNF | UNF | CNF |
| | Compare Undegraded T-RNAs | PAFR | C-HKR | SCR | — |
| (1) | Combine Table 92 Design Solutions: | | | | |
| | Preferred | | | | |
| | (a) 1a, 5a, 6a, 7a, 9b, 10c, 11a, 12a, 13a, | | | | |
| | 14, 15a, 18a, 19, 21, 22, 25, 26b, or | | | | |
| | (b) As (1a), except use Design Solution | | | | |
| | 1b instead of Design Solution 1a, or | | | | |
| | (c) As (1a-b), except delete Design | | | | |
| | Solution 5b instead of Design Solution | | | | |
| | 5a, or | | | | |
| | (d) As (1a-c), except use Design Solution | | | | |
| | 7b instead of Design Solution 7a, or | | | | |
| | (e) As (1a-d), except use Design Solution | | | | |
| | 8a or 8b instead of Design Solution 7a | | | | |
| | or 7b, or | | | | |
| | (f) As (1a-e), except use Design Solution | | | | |
| | 9a instead of Design Solution 9b, or | | | | |
| | (g) As (1a-f), except use Design Solution | | | | |
| | 10a or 10b instead of Design Solution | | | | |
| | 10c, or | | | | |
| | (h) As (1a-c, f-g), except use Design | | | | |
| | Solution 11b instead of Design Solution | | | | |
| | 11a, or | | | | |
| | (i) As (1a-g), except use Design Solution | | | | |
| | 12b instead of Design Solution 12a | | | | |
| | Compare Undegraded Isolated mRNA | — | C-HKR | SCR | — |
| (2) | Combine Table 92 Design Solutions: | | | PAFR | |
| | Other | | | | |
| | (a) As (1a-g), except use Design Solution | | | | |
| | 15b instead of Design Solution 15a | | | | |

TABLE 94
Preferred and Other Practices for Design Solution Combinations Which Can Be
Known to Provide Improved Normalization of Dot Blot Assay Particular Gene RASR
Values for All, or One or More, Pertinent UNFs and/or CNFs

| | | NFs Which Can Be | | Pertinent NFs To Be | |
|---|---|---|---|---|---|
| | | Ignored For | | Determined and | |
| | | Normalization | | Normalized For | |
| Combination of Assay Design Solutions | | UNF | CNF | UNF | CNF |
| | Compare Undegraded T-RNAs | PAFR | C-HKR | SCR | — |
| (1) | Combine Table 92 Design Solutions: | MLDR | | | |
| | Preferred | | | | |
| | (a) 2a, 5a, 6a, 7a, 9b, 10a, 11a, 12a, | | | | |
| | 13a, 14, 15a, 18a, 19, 21, 23, 25, | | | | |
| | 26b, or | | | | |
| | (b) As (1a), except use Design Solution | | | | |
| | 5b instead of Design Solution 5a, | | | | |
| | or | | | | |
| | (c) As (1a-b), except delete Design | | | | |
| | Solution 7b instead of Design | | | | |
| | Solution 7a, or | | | | |
| | (d) As (1a-c), except use Design | | | | |
| | Solution 8a or 8b instead of Design | | | | |
| | Solution 7a or 7b, or | | | | |
| | (e) As (1a-d), except use Design | | | | |
| | Solution 9a instead of Design | | | | |
| | Solution 9b, or | | | | |
| | (f) As (1a-e), except use Design | | | | |
| | Solution 10b or 10c instead of | | | | |
| | Design Solution 10a, or | | | | |
| | (g) As (1a-b, e-f), except use Design | | | | |
| | Solution 11b instead of Design | | | | |
| | Solution 11a, or | | | | |
| | (h) As (1a-g), except use Design | | | | |
| | Solution 12b instead of Design | | | | |
| | Solution 12a | | | | |
| (2) | Combine Table 92 Design Solutions: | PAFR | C-HKR | SCR | — |
| | Other | MLDR | | | |
| | (a) As (1a-h), except use Design | | | | |
| | Solution 13b instead of Design | | | | |
| | Solution 13a | | | | |
| | Compare Undegraded Isolated mRNA | MLDR | C-HKR | SCR | — |
| (3) | Combine Table 92 Design Solutions: | | | PAFR | |
| | Preferred | | | | |
| | 2a, 5a, 6a, 7a, 9b, 10a, 11a, 12a, 13a, 14, | | | | |
| | 15b, 18a, 19, 21, 23, 25, 26b | | | | |
| | or | | | | |
| | (b) As (3a), except use Design Solution | | | | |
| | 5b instead of Design Solution 5a | | | | |
| | or | | | | |
| | (c) As (3a-b), except delete Design | | | | |
| | Solution 7b, or 8a, or 8b instead of | | | | |
| | Design Solution 7a | | | | |
| | or | | | | |
| | (d) As (3a-c), except use Design Solution | | | | |
| | 9a instead of Design Solution 9b | | | | |
| | or | | | | |
| | (e) As (3a-d), except use Design Solution | | | | |
| | 10b or 10c instead of Design Solution | | | | |
| | 10a | | | | |
| | or | | | | |
| | (f) As (3a-b, d-e), except use Design | | | | |
| | Solution 11b instead of Design | | | | |
| | Solution 11a | | | | |
| | or | | | | |
| | (g) As (3a-f), except use Design Solution | | | | |
| | 12b instead of Design Solution 12a | | | | |
| (4) | Combine Table 92 Design Solutions: | MLDR | — | SCR | C-HKR |
| | Other | | | PAFR | |
| | (a) As (3a-g), except use Design Solution | | | | |
| | 13b and 26 instead of Design Solution | | | | |
| | 13a | | | | |
| | Compare Degraded T-RNAs | MLDR | C-HKR | SCR | — |
| (5) | Combine Table 92 Design Solutions: | PAFR | | | |
| | Preferred | | | | |
| | (a) 2a, 5a, 6a, 7a, 9b, 10a, 11a, 12a, 13a, | | | | |
| | 14, 15a, 16a or b, 17a or b, 18a or b, | | | | |
| | 23, 25, 26b | | | | |
| | or | | | | |
| | (b) As (5a), except use Design Solution | | | | |
| | 5b instead of Design Solution 5a | | | | |
| | or | | | | |
| | (c) As (5a-b), except use Design Solution | | | | |
| | 9a instead of Design Solution 9b | | | | |
| | or | | | | |
| | (d) As (5a-c), except use Design Solution | | | | |
| | 10b instead of Design Solution 10a | | | | |
| | or | | | | |
| | (e) As (5a-d), except use Design Solution | | | | |
| | 11b instead of Design Solution 11a | | | | |
| (6) | Combine Table 92 Design Solutions: | MLDR | C-HKR | SCR | — |
| | Other | PAFR | | | |
| | (a) As (5a-e), except use Design Solution | | | | |
| | 13b instead of Design Solution 13a | | | | |
| | Compare Isolated mRNA Produced from | MLDR | C-HKR | SCR | — |
| | Degraded T-RNAs | | | PAFR | |
| (7) | Combine Table 92 Design Solutions: | | | | |
| | Other | | | | |
| | As (5a-c, e), except use Design | | | | |
| | Solution 15b instead of Design | | | | |
| | Solution 15a | | | | |
| (8) | Combine Table 92 Design Solutions: | MLDR | C-HKR | SCR | — |
| | Other | | | PAFR | |
| | As (7a), except use Design Solution | | | | |
| | 13b instead and 26 instead of Design | | | | |
| | Solution 13a | | | | |
| | Compare Degraded T-RNAs | MLDR | C-HKR | SCR | — |
| (9) | Combine Table 92 Design Solutions: | PAFR | | | |
| | Preferred | | | | |
| | (a) 2a, 5a, 6a, 8b, 9b, 10c, 11a, 12a, 13a, | | | | |
| | 14, 15a, 16a 17a, 18a, 19, 23, 25, 26b, | | | | |
| | or | | | | |
| | (b) As (9a), except use Design Solution | | | | |
| | 5b instead of Design Solution 5a, or | | | | |
| | (c) As (9a-b), except use Design Solution | | | | |
| | 9a instead of Design Solution 9b, or | | | | |
| | (d) As (9a-c), except use Design Solution | | | | |
| | 12b instead of Design Solution 12a | | | | |
| (10) | Combine Table 92 Design Solutions: | MLDR | C-HKR | SCR | — |
| | Other | PAFR | | | |
| | (a) As (9a-d), except use Design Solution | | | | |
| | 13b and 26 Instead and 26 instead of | | | | |
| | Design Solution 13a | | | | |
| (11) | Combine Table 92 Design Solutions: | MLDR | C-HKR | SCR | — |
| | Preferred | PAFR | | | |
| | 2a, 5a, 6a, 8a, 9a or b, 10c, 11a, 12a, 13a, | | | | |
| | 14, 15a, 16b, 18b, 23, 24, 25, 26b, or | | | | |
| | (b) As (11a), except use Design Solution | | | | |
| | 5b instead of Design Solution 5a | | | | |
| (12) | Combine Table 92 Design Solutions: | MLDR | C-HKR | SCR | — |
| | Other | PAFR | | | |
| | (a) As (11a-b), except use Design | | | | |
| | Solution 13b and 26 instead of Design | | | | |
| | Solution 13a | | | | |
| (13) | Combine Table 92 Design Solutions: | PAFR | C-HKR | SCR | — |
| | Other | | | MLDR | |
| | (a) As (11a-b), except delete Design | | | | |
| | Solution 24 | | | | |
| | Compare Isolated mRNAs Produced from | MLDR | C-HKR | SCR | — |
| | Degraded T-RNAs | | | PAFR | |
| (14) | Combine Table 92 Design Solutions: | | | | |
| | Preferred | | | | |
| | (a) 2a, 5a, 6a, 8a, 9a or b, 10c, 11a, | | | | |
| | 12a, 13a, 14, 15b, 17b, 18b, 23, 25, 26b | | | | |
| (15) | Combine Table 92 Design Solutions: | — | C-HKR | SCR | — |
| | Other | | | PAFR | |
| | (a) 2a, 5a, 6a, 8a, 9a or b, 10c, 11a, | | | MLDR | |
| | 12a, 13a, 14, 15b, 17b, 18b, 23, 24, 25 | | | | |
| (16) | Combine Table 92 Design Solutions: | — | C-HKR | SCR | — |
| | Other | | | PAFR | |
| | (a) As (15a), except use Design | | | MLDR | |
| | Solution 13b and 26 instead of Design | | | | |
| | Solution 13a | | | | |

TABLE 95

| | NFs Which Can Be | | Pertinent NFs To | |
|---|---|---|---|---|
| | Ignored For | | Be Determined and | |
| | Normalization | | Normalized For | |
| Combination of Assay Design Solutions | UNF | CNF | UNF | CNF |

Preferred and Other Practices for Design Solution Combinations Which Can Be
Known to Provide Improved Normalization of Nuclease Protection Assay Particular
Gene Comparison RASR Values for All, or One or More, Pertinent UNFs and/or
CNFs

| | | | | | |
|---|---|---|---|---|---|
| | Comparison of Undegraded T-RNAs | MLDR | C-HKR | SCR | — |
| (1) | Combine Table 92 Design Solutions: Preferred | PAFR | | | |
| | (a) 3a, 5a, 6a, 7a, 9a, 10a, 11a, 12a, 13b, 14, 15a, | | | | |
| | 18, 19, 21, 25, 26b, or | | | | |
| | (b) As (1a), except use Design Solution 5b | | | | |
| | instead of Design Solution 5a, or | | | | |
| | (c) As (1a-b), except delete Design Solution 7b | | | | |
| | instead of Design Solution 7a, or | | | | |
| | (d) As (1a-c), except use Design Solution 8a or | | | | |
| | 8b instead of Design Solution 7a or 7b, or | | | | |
| | (e) As (1a-d), except use Design Solution 9b | | | | |
| | instead of Design Solution 9a, or | | | | |
| | (f) As (1a-e), except use Design Solution 10b or | | | | |
| | 10c instead of Design Solution 10a, or | | | | |
| | (g) As (1a-f), except use Design Solution 3b and | | | | |
| | 6b instead of Design Solution 3a and 6a | | | | |
| | Comparison of Degraded T-RNAs | MLDR | C-HKR | SCR | — |
| (2) | Combine Table 92 Design Solutions: Preferred | PAFR | | | |
| | (a) As (1a-g), except use degraded T-RNAs, or | | | | |
| | (b) As (2a), except use Design Solution 16b, or | | | | |
| | (c) As (2a-b), except use Design Solution 18b | | | | |
| | instead of Design Solution 18a | | | | |

Preferred and Other Practices for Design Solution Combinations Which Can
Be Known to Provide Improved Normalization of Nuclease Protection Assay
Particular Gene RASR Values for All, or One or More, Pertinent UNFs and/or CNFs

| | | | | | |
|---|---|---|---|---|---|
| | Comparison of Undegraded Isolated mRNA | MLDR | C-HKR | SCR | — |
| (3) | Combine Table 92 Design Solutions: Preferred | | | PAFR | |
| | (a) As (1a-g), except use Design Solution 15b | | | | |
| | instead of Design Solution 15a | | | | |
| | Comparison of Isolated mRNAs Produced from | MLDR | C-HKR | SCR | — |
| | Degraded T-RNAs | | | PAFR | |
| (4) | Combine Table 92 Design Solutions: Preferred | | | | |
| | (a) 3a, 5a, 6a, 7a, 9a, 10a, 11a, 12a, 13b, 14, 15b, | | | | |
| | 17a, 18a, 25, 26b, or | | | | |
| | (b) As (4a), except use Design Solution 5b | | | | |
| | instead of Design Solution 5a, or | | | | |
| | (c) As (4a-b), except delete Design Solution 9b | | | | |
| | instead of Design Solution 9a, or | | | | |
| | (d) As (4a-c), except use Design Solution 17b | | | | |
| | instead of Design Solution 17a, or | | | | |
| | (e) As (4a-d), except use Design Solution 18b | | | | |
| | instead of Design Solution 18a, or | | | | |
| | (f) As (4a-e), except use Design Solution 3b and | | | | |
| | 6b instead of Design Solution 3a and 6a | | | | |
| (5) | Combine Table 92 Design Solutions: Other | — | C-HKR | SCR | — |
| | (a) 3a, 5a, 6a, 8a, 9a, 10c, 11a, 12a, 13b, 14, 15b, | | | PAFR | |
| | 17b, 18b, 25, 26b, or | | | MLDR | |
| | (b) As (5a), except use Design Solution 5b | | | | |
| | instead of Design Solution 5a, or | | | | |
| | (c) As (5a-b), except delete Design Solution 7b | | | | |
| | or 8b instead of Design Solution 8a, or | | | | |
| | (d) As (5a-c), except use Design Solution 9b | | | | |
| | instead of Design Solution 9a | | | | |
| (6) | Combine Table 92 Design Solutions: | MLDR* | C-HKR⁺ | SCR⁺ | — |
| | Other | | | PAFR⁺ | |
| | (a) As (5a-d), except use Design Solution | | | | |
| | 18b is deleted and Design Solutions | | | | |
| | 18a, 20, 21 apply for a subset of | | | | |
| | particular gene mRNAs which are | | | | |
| | undegraded in the isolated mRNA | | | | |

*Applies only to a compared particular gene mRNA which is short enough in undegraded nucleotide length to be undegraded in each compared isolated mRNA prep even though the T-RNApreps are degraded.
⁺Applies to all particular gene mRNAs in assay.

The known design solution combination associated with a non-microarray northern blot, dot blot or nuclease protection assay, determines whether the assay can be known to be associated with improved normalization of the non-microarray assay measured particular gene RN or mTN, mRNA abundance or DGER values, and the degree to which the normalization and results can be known to be improved, relative to prior art non-microarray normalization practice. As discussed, prior art does not determine the non-microarray assay values for the UNFs SCR, PAFR, and MLDR, and normalize for them. Further, the prior art non-microarray practice does not provide the information necessary for determining the design solution combination associated with a specific prior art non-microarray assay. Thus, the design solution combination associated with any specific prior art non-microarray assay is not known, and probably cannot be completely known retrospectively for many, if any, prior art assays. This means that except for those few prior art microarray assays which are known to be not normalized for certain pertinent UNFs, the completeness and validity of normalization for most prior art microarray assay measured and normalized particular gene RN or mTN, mRNA abundance or N-DGER values, is unknown. Consequently, absent further information, these results are uninterpretable with regard to, biological accuracy, to the quantitative aspects of gene expression extent, and to the direction of gene regulation change. It is possible, but not likely, that unknown to the prior art non-microarray practice, a particular prior art non-microarray assay is associated with incomplete but improved normalization, or with complete normalization. However, absent knowledge of the design solution combination associated with the prior art non-microarray assay, it cannot be known whether the assay is associated with improved normalization or results or not.

The design solution combination associated with a non-microarray assay determines the following. (i) the validity of pertinent CNF normalization. (ii) the completeness of normalization for pertinent UNFs and CNFs. (ii) the ease of determining the assay values for the pertinent UNFs and CNFs. (iv) ease and simplicity of the normalization process. (v) biological accuracy of the assay measured and normalized particular gene RN, mRNA abundance, and N-DGER values. (vi) the overall interpretability of assay measured and normalized particular gene mTN, mRNA abundance and N-DGER values. (vii) the between and within assay intercomparability of the non-microarray assay measured particular gene RN, mRNA abundance and N-DGER values. (viii) the intercomparability of the non-microarray assay measured cell sample particular gene RN, mRNA abundance, and N-DGER values, with cell sample particular gene RN, mRNA abundance, or N-DGER values, obtained with a different microarray or non-microarray method for gene expression analysis, for which the design solution combination associated with the assay is known. It is desirable to maximize each of these characteristics as much as possible. Here, if the non-microarray assay measured particular gene RN, mRNA abundance or N-DGER value is biologically accurate, then, the normalization is valid and complete, and the particular gene RN, mRNA abundance or N-DGER value, can be validly interpreted, and validly intercompared with other biologically accurate RT-PCR, microarray, or other non-microarray particular gene RN, mRNA abundance, or N-DGER values. Further, such biologically accurate non-microarray assay measured particular gene RN, mRNA abundance or N-DGER values, can be validly used to corroborate and validate particular gene RN, mRNA abundance, or N-DGER values, measured by micro-array or other non-microarray assay methods.

As indicated in Tables 93 through 95, these non-microarray northern blot, dot blot, and nuclease protection methods, can be associated with the UNFs SCR, PAFR and MLDR. As indicated in Table 93(1a), Table 94(1a), Table 95(1a), and elsewhere, such a non-microarray assay can be designed so that the MLDR and PAFR UNFs can be known to equal one, and therefore can be ignored during normalization. However, the assay SCR UNF value must always be determined. Note that not all assay variables which have been identified and used by the prior art for the normalization of these assays, are considered here in these Table 93 through 95 design solution combinations. Prior art appears to validly normalize for these omitted assay variables, and here it is assumed that this is done.

A non-microarray assay can be described by the design solution combination which is associated with the assay. An accurate assay design solution combination description serves as the basis for identifying the following. (i) the pertinent UNFs and CNFs which are associated with the assay. (ii) the pertinent UNFs and CNFs which can be ignored during the assay normalization process. (iii) the pertinent UNFs and CNFs assay values which must be determined and normalized for in the assay. (iv) the pertinent UNFs and CNFs which can be determined and normalized for. (v) the pertinent UNFs and CNFs which are normalized for. (vi) the assumptions necessary to determine UNF and CNF assay values. Such an overall description is necessary in order to evaluate the utility, biological accuracy, and intercomparability, of the assay measured particular gene comparison RN, mRNA abundance, and N-DGER values. Such an overall description should be available for every non-microarray assay. Such an overall design solution combination description can be used to plan future non-microarray assays, and to interpret already existing non-microarray assay particular gene comparison RN, mRNA abundance or NASR values. Such overall design solution combination descriptions were not created for prior art non-microarray assays of any kind. In addition such an overall design solution combination description will allow the effective standardization of non-microarray assay formats.

Improvement of RT-PCR Assay Normalization Process.

One or another RT-PCR method is widely used for prior art gene expression analysis and comparison, and is often used by the prior art to validate microarray measured and normalized particular gene NASR and N-DGER values. These prior art RT-PCR assays are also associated with pertinent UNFs and CNFs. However, relative to microarray assays these RT-PCR assays appear to be associated with a smaller number of pertinent UNFs or CNFs. Prior art RT-PCR assay design is not standardized, and there are a variety of different assay designs which are commonly used. Table 96 describes different prior art RT-PCR assay situations and the pertinent UNFs and CNFs which must be accurately determined and normalized for during the normalization process, in order to obtain improved RT-PCR assay measured particular gene RN or mTN values, or mRNA abundance values, or N-DGER values.

TABLE 96
UNFs and CNFs Associated with Prior Art RT-PCR Assay Particular
Gene N-DGER Determinations

| | UNFs and CNFs Which May Be Pertinent When | | | |
|---|---|---|---|---|
| | Comparing Cell | | | |
| | Sample | | | |
| | | | Undegraded | Degraded |
| | Undegraded | Degraded | Isolated | Isolated |
| Primer Used | T-RNAs | T-RNAs | mRNA | mRNA |
| SG | SCR | SCR | SCR | SCR |
| or | PG AE•SER | PG AE•SER | PAFR | PAFR |
| Random | S AE•SER | S AE•SER | PG AE•SER | PG AE•SER |
| | PG AE•AER | PG AE•AER | S AE•SER | S AE•SER |
| | S AE•AER | S AE•AER | PG AE•AER | PG AE•AER |
| | | | S AE•AER | S AE•AER |
| Oligo dT | SCR | SCR | SCR | SCR |
| | PAFR | PAFR | PAFR | PAFR |
| | PG AE•SER | PG AE•SER | PG AE•SER | PG AE•SER |
| | S AE•SER | S AE•SER | S AE•SER | S AE•SER |
| | PG AE•AER | PG AE•AER | PG AE•AER | PG AE•AER |
| | S AE•AER | S AE•AER | S AE•AER | S AE•AER |

PG = Particular Gene
S = Standard

The determination of the assay values, for the UNFs and CNFs, and their use for normalization, were described earlier. The large majority of prior art relative and absolute quantitation RT-PCR assays utilize internal or external standards, or both, in order to quantitate. For a specific prior art RT-PCR assay, the particular gene and standard SCR and PAFR UNF assay values are not determined. The actual particular gene and standard AE•SER and AE•AER CNF assay values are only rarely determined and considered during the normalization process for a specific prior art RT-PCR assay. Prior art does, albeit infrequently, normalize for predetermined or average particular gene and standard AE•AE values. For a cell sample particular gene comparison, the particular gene AE•SER value is required to determine the assay SCR value for the PCR amplification step. For an RT-PCR cell sample particular gene (PG) comparison which uses an exogenous standard mRNA in each cell sample, the standard AE•SER value is believed to be the same as the PG AE•SER value. Note that for RT-PCR assays which use a single external mRNA standard quantitative calibration curve to determine the particular gene N-DGER value, the external standard AE•SER value may not equal one. Note further that for RT-PCR particular gene comparison assays, the cell sample particular gene AE•SE values are used to determine both the particular gene RN value and the number of particular gene ACEs present in the PCR step. For those RT-PCR particular gene comparison assays which utilize an external, or exogenous, or endogenous internal standard for quantitation, the standard AE•SE value is necessary in order to determine the validity of the use of the standard, and to normalize for differences in the particular gene and standard AE•SE values. Because: the prior art practice for the determination of and normalization for particular gene or standard AE•SER and AE•AER assay values is essentially invalid; and because these CNFs, and in particular the AE•AER, can have such a large effect on the biological accuracy of the assay measured particular gene N-DGER value; the AE•SE and AE•AE CNFs have been included in Table 96, and are considered to be qualified design solutions for improving the prior art RT-PCR assay normalization process, and the assay measured particular gene NASR values. Other prior art RT-PCR assay associated CNFs exist. Such CNFs can vary for different RT-PCR assay designs. The prior art normalization practice for these CNFs appears to be valid, and it is here assumed that this is true. Therefore, these CNFs are not considered here.

Prior art RT-PCR assay practice does not determine the assay value for, or normalize particular gene comparison RASR values for, the pertinent UNFs SCR and PAFR. Each of these UNFs can cause an assay measured particular gene comparison N-DGER value to deviate significantly from biological accuracy when the UNF value deviates significantly from one. The previously discussed estimates in Table 51 for the magnitudes of the deviation from one which are believed to commonly occur for the PAFR and SCR UNFs, also apply to prior art RT-PCR assays. It is likely that many prior art RT-PCR assays are associated with such a UNF which deviates significantly from one. Such deviations have practical meaning and importance since prior art practice assay measurement accuracies of ±1.2 to ±2 fold are often claimed by the prior art. For the CNF AE•SER, a deviation from one of 1.5 fold is believed to be common for a prior art RT-PCR assay, while for the CNF AE•AER, a deviation from one of 3-6 fold or more, is likely to be common.

In order to know whether an RT-PCR assay measured particular gene N-DGER value needs to be normalized for the particular gene comparison SCR or PAFR values, or the particular gene comparison or standard AE•SER and AE•AER values, it is necessary to first identify each UNF or CNF which is pertinent to the assay. Then, a measure of the assay value for each pertinent NF must be determined in order to determine whether normalization for that UNF or CNF is necessary. Then, if necessary, the particular gene RASR value should be normalized for each pertinent SCR, PAFR, AE•SER and AE•AER value. For a typical prior art RT-PCR assay, the requirement to determine and normalize for the pertinent UNFs and CNFs very significantly increases the complexity and effort associated with the typical assay, relative to a prior art RT-PCR assay. Determination of assay values for particular gene and standard AE•SE and AE•AE values is complex and labor intensive, and the level of measurement accuracy of the AE•AE values must be high. In addition, measurement error and noise will be associated with each experimentally determined UNF and CNF value and its use for normalization.

These considerations make it very desirable, if not necessary, to simplify the determination of pertinent UNF assay values and the normalization process as much as possible, and to eliminate the need to experimentally determine assay values for as many UNFs as possible. Here it is particularly desirable to eliminate the need to experimentally determine the assay values for those UNFs which are difficult or labor intensive to determine, such as the PAFR.

Earlier sections extensively discussed the underlying basis for each assay NF, and the assay situations under which each NF is pertinent. As a result of this, it is possible to identify the assay factors which can and must be controlled for different assay situations, in order to simplify the process for determining the pertinent UNF and CNF assay values and normalizing for them. This knowledge makes it possible to design non-microarray assays which do not require the direct determination of certain pertinent UNFs in order to know that such UNFs are validly normalized for. The overall result of such assay designs is a simplified version of the improved RT-PCR assay normalization process. This can be accomplished by judicious assay design and measurement, as is discussed below.

The various design approaches which will result in an improved normalization process relative to prior art normalization processes, are presented in Table 52. The successful implementation of any one of the Table 52 design approaches 1-8, will produce a normalization process which can be known to be improved, relative to prior art RT-PCR assay normalization practices. The successful implementation of Table 52 design approach 9, will produce RT-PCR assay results which are known to contain fewer NF related false negative results than prior art RT-PCR assay results.

Prior art RT-PCR assay design is not standardized, and there are a variety of different assay designs which are commonly used for each particular RT-PCR method. The improvement of the normalization process for each of the alternate assay designs will be discussed. Design components or design solutions which can be used to produce improved RT-PCR assay normalization and improved RT-PCR assay measured particular gene N-DGER values, are presented in Table 92. Each of these design solutions or design solution components reflects an aspect of RT-PCR assay design which directly or indirectly impacts on an assay pertinent CNF or UNF.

Different combinations of these designs can be used to describe an overall RT-PCR assay which is improved, relative to prior art RT-PCR assays. Certain of these design solutions are discussed and further defined below. Here, design solution will be termed DS. DS 4. Most prior art RT-PCR assays involve the analysis of one particular gene mRNA. DS 15. Most prior art RT-PCR assays analyze cell sample T-RNA. DS 25. This will minimize or eliminate the presence of UNF or CNF related false negative results and their associated RDMs. DS 27. Prior art frequently uses all three of these primer types, but SG primers are the most widely used. DS 28, 37. This provides the maximum primer use flexibility, and allows the use of any of the three primer types even for degraded mRNA. DS 29, 30, 31, 32. Each of these can positively affect the magnitude and reproducibility of the particular gene or standard PCR amplicon amplification efficiency E and thereby the AE•AE values. DS 33, 40. These apply to an assay situation where an assay standard is not used. This approach will be preferable to using an assay standard, if the measurement errors involved with determining the assay standard AE•SE and AE•AE values are high enough. E and AE•AE values appear to be very difficult to measure accurately and reproducibly. DS 34, 41. Prior art almost always uses this approach. DS 35, 42. Given the variability associated with prior art AE•SE and AE•AE measurements, this approach is preferred to that of DS 34, 41. DS 36, 38, 39. Each of these can positively affect the ability to accurately and reproducibly measure the particular gene or standard AE•AE assay values.

Relative to prior art normalization practice, the normalization of RT-PCR measured particular gene mTN, RN, mRNA abundance, or N-DGER values, is improved when one or more of these assay measured values produced by the RT-PCR assay is known to be validly normalized for one or more of the following. (i) one or more pertinent UNFs. (ii) one or more pertinent UNFs and one or more pertinent CNFs. (iii) one or more pertinent UNFs and all pertinent CNFs. (iv) all pertinent UNFs and CNFs. For an RT-PCR assay the preferred improved normalization process assay design results in the valid normalization of one or more assay measured particular gene mTN, RN, or mRNA abundance, or DGER value for all pertinent UNFs and CNFs, and also results in minimizing the number of UNF and CNF related false negative results and their associated RDMs, which are associated with the assay. Such assay designs are described below. A variety of different RT-PCR assay formats are practiced by the prior art, and different formats can be associated with different combinations of pertinent UNFs and CNFs. For simplicity each different prior art general assay design will be discussed in terms of the Table 92 design solution combinations which can be known to provide the improved normalization of the RT-PCR assay measured gene expression analysis results for the pertinent UNFs and CNFs. These preferred practice design solution combinations are presented in Table 97.

TABLE 97
Preferred and Other Design Solution Combination Practices Which Can Be Known to
Provide Improved Normalization of RT-PCR Assay Results

| | | NFs Which Can Be | | Pertinent NFs To Be | |
|---|---|---|---|---|---|
| | | Ignored For | | Determined and | |
| Combination of RT-PCR Assay Design | | Normalization | | Normalized For | |
| Solutions | | UNF | CNF | UNF | CNF |
| A. | Compare Cell Sample T-RNAs | PAFR | — | SCR | PG AE•SE |
| | Combine Table 92 Design Solutions: | | | | PG AE•AE |
| | Preferred Practices When Standard is Not | | | | |
| | Used | | | | |
| | (1) Use SG Primed cDNA | | | | |
| | (a) 4a or 4b, 15a, 25, 26b, 27a, 28, 29, | | | | |
| | 31, 32, 33, 36-39, | | | | |
| | 40 | | | | |
| | (2) Use Oligo dT Primed cDNA | — | — | SCR | PG AE•SE |
| | (a) 4a or 4b, 15a, 25, 26b, 27b, 29, 31, | | | PAFR | PG AE•AE |
| | 32, 33, 36-39, 40 | | | | |
| | (3) Use Random Primed cDNA | PAFR | — | SCR | PG AE•SE |
| | (a) 4a or 4b, 15a, 25, 26b, 27c, 29, 31, | | | | PG AE•AE |
| | 33, 36, 38, 39, 40 | | | | |
| B. | Compare Cell Sample Isolated mRNAs | — | — | SCR | PG AE•SER |
| | Combine Table 92 Design Solutions: | | | PAFR | PG AE•AER |
| | Preferred Practices When Standard is Not | | | | |
| | Used | | | | |
| | (1) Use SG Primed cDNA | | | | |
| | (a) 4a or 4b, 15b, 25, 26b, 27a, 28, 29, | | | | |
| | 31, 32, 33, 36-39, | | | | |
| | 40 | | | | |
| | (2) Use Oligo dT Primed cDNA | — | — | SCR | PG AE•SER |
| | (a) 4a or 4b, 15b, 25, 26b, 27b, 29, 31, | | | PAFR | PG AE•AER |
| | 32, 33, 36-39, 40 | | | | PG AE•AER |
| | (3) Use Random Primed cDNA | — | — | SCR | PG AE•SER |
| | (a) 4a or 4b, 15b, 25, 26b, 27c, 29, | | | PAFR | PG AE•AER |
| | 31, 33, 36-39, 40 | | | | |
| C. | Compare Cell Sample T-RNAs | PAFR | — | SCR | PG AE•SER |
| | Combine Table 92 Design Solutions: | | | | S AE•SER |
| | Preferred | | | | PG AE•AER |
| | Practices When Standard(s) Are Used | | | | S AE•AER |
| | (1) Use SG Primed cDNA | | | | |
| | (a) 4a or 4b, 15a, 25, 26b, 27a, 28, | | | | |
| | 29, 30, 31, 32, 34, | | | | |
| | 36-39, 41 | | | | |
| | (2) Use Oligo dT Primed cDNA | — | — | SCR | PG AE•SER |
| | (a) 4a or 4b, 15a, 25, 26b, 27b, 29, | | | PAFR | S AE•SER |
| | 30, 31, 32, 34, 36-39, | | | | PG AE•AER |
| | 41 | | | | S AE•AER |
| | (3) Use Random Primed cDNA | PAFR | — | SCR | PG AE•SER |
| | (a) 4a or 4b, 15a, 25, 26b, 27c, 29, | | | | S AE•SER |
| | 30, 31, 34, 36, 38, | | | | PG AE•AER |
| | 39, 41 | | | | S AE•AER |
| D. | Compare Cell Sample Isolated mRNAs | — | — | SCR | PG AE•SER |
| | Combine Table 92 Design Solutions: | | | PAFR | S AE•SER |
| | Preferred Practices When Standard(s) Are | | | | PG AE•AER |
| | Used | | | | S AE•AER |
| | (1) Use SG Primed cDNA | | | | |
| | (a) 4a or 4b, 15b, 25, 26b, 27a, 28, | | | | |
| | 29, 30, 31, 32, 34, | | | | |
| | 36-39, 41 | | | | |
| | (2) Use Oligo dT Primed cDNA | — | — | SCR | PG AE•SER |
| | (a) 4a or 4b, 15b, 25, 26b, 27b, 29, | | | PAFR | S AE•SER |
| | 30, 31, 32, 34, 36-39, | | | | PG AE•AER |
| | 41 | | | | S AE•AER |
| | (3) Use Random Primed cDNA | — | — | SCR | PG AE•SER |
| | (a) 4a or 4b, 15b, 25, 26b, 27c, 29, 30, | | | PAFR | S AE•SER |
| | 31, 34, 36-39, 40 | | | | S AE•AER |
| E. | Compare Cell Sample T-RNAs | PAFR | — | SCR | PG AE•SER |
| | Combine Table 92 Design Solutions: | | | | S AE•SER |
| | Preferred Practices When Standard(s) Are | | | | PG AE•AER |
| | Used | | | | S AE•AER |
| | Use SG Primed cDNA | | | | |
| | (a) 4a or 4b, 15a, 25, 26b, 27a, 28, 29, | | | | |
| | 30, 31, 32, 35, 36-39, 42 | | | | |
| | (1) Use Oligo dT Primed cDNA | — | — | SCR | PG AE•SER |
| | (a) 4a or 4b, 15a, 25, 26b, 27b, 29, | | | PAFR | S AE•SER |
| | 30, 31, 32, 35, 36-39, | | | | PG AE•AER |
| | 42 | | | | S AE•AER |
| | (2) Use Random Primed cDNA | PAFR | — | SCR | PG AE•SER |
| | (a) 4a or 4b, 15a, 25, 26b, 27c, 29, | | | | S AE•SER |
| | 30, 31, 35, 36, 38, | | | | PG AE•AER |
| | 39, 42 | | | | S AE•AER |
| F. | Compare Cell Sample Isolated mRNAs | — | — | SCR | PG AE•SER |
| | Combine Table 92 Design Solutions: | | | PAFR | S AE•SER |
| | Preferred Practices When Standard(s) Are | | | | PG AE•AER |
| | Used | | | | S AE•AER |
| | (1) Use SG Primed cDNA | | | | |
| | (a) 4a or 4b, 15b, 25, 26b, 27a, 28, 29, | | | | |
| | 30, 31, 32, 35, 36-39, 42 | | | | |
| | (2) Use Oligo dT Primed cDNA | — | — | SCR | PG AE•SER |
| | (a) 4a or 4b, 15b, 25, 26b, 27b, 29, 30, | | | PAFR | S AE•SER |
| | 31, 32, 35, 36-39, 42 | | | | PG AE•AER |
| | | | | | S AE•AER |
| | (3) Use Random Primed cDNA | — | — | SCR | PG AE•SER |
| | (a) 4a or 4b, 15b, 25, 26b, 27c, 29, | | | PAFR | S AE•SER |
| | 30, 31, 35, 36-39, | | | | PG AE•AER |
| | 42 | | | | S AE•AER |
| G. | Compare Cell Sample T-RNAs | PAFR | — | SCR | PG AE•SER |
| | Combine Table 92 Design Solutions: | | | | S AE•AER |
| | Other | | | | PG AE•AER |
| | Practices When Standard is Not Used | | | | S AE•AER |
| | (1) Use SG Primed cDNA | | | | |
| | (a) 4a or 4b, 15a, 27a, 33, 40 | | | | |
| | (2) Use Oligo dT Primed cDNA | — | — | SCR | PG AE•SER |
| | (a) 4a or 4b, 15a, 27b, 33, 40 | | | PAFR | S AE•SER |
| | | | | | PG AE•AER |
| | | | | | S AE•AER |
| | (3) Use Random Primed cDNA | PAFR | — | SCR | PG AE•SER |
| | (a) 4a or 4b, 15a, 27c, 33, 40 | | | | S AE•SER |
| | | | | | PG AE•AER |
| | | | | | PG AE•AER |
| H. | Compare Cell Sample T-RNAs | PAFR | — | SCR | PG AE•SER |
| | Combine Table 92 Design Solutions: | | | | S AE•SER |
| | Other | | | | S AE•AER |
| | Practices When Standard(s) Are Used | | | | S AE•AER |
| | (1) Use SG Primed cDNA | | | | |
| | (a) 4a or 4b, 15a, 27a, 34, 41 | | | | |
| | or | | | | |
| | (b) 4a or 4b, 15a, 27a, 35, 42 | | | | |
| | (2) Use Oligo dT Primed cDNA | — | — | SCR | PG AE•SER |
| | (a) 4a or 4b, 15a, 27b, 34, 41 | | | PAFR | S AE•SER |
| | or | | | | PG AE•AER |
| | (b) 4a or 4b, 15a, 27b, 35, 42 | | | | S AE•AER |
| | (3) Use Random Primed cDNA | PAFR | — | SCR | PG AE•SER |
| | (a) 4a or 4b, 15a, 27c, 34, 41 | | | | S AE•SER |
| | or | | | | PG AE•AER |
| | (b) 4a or 4b, 15a, 27c, 35, 42 | | | | S AE•AER |
| I. | Compare Cell Isolated mRNAs | — | — | SCR | PG AE•SER |
| | Combine Table 92 Design Solutions: | | | PAFR | S AE•SER |
| | Other Practices When Standard(s) Are | | | | PG AE•AER |
| | Used | | | | S AE•AER |
| | (1) Use SG, or Oligo dT, or Random | | | | |
| | Primed, cDNA | | | | |
| | (a) 4a or 4b, 15b, 27a or 27b or 27c, | | | | |
| | 34, 41 | | | | |
| | or | | | | |
| | (b) 4a or 4b, 15b, 27a or 27b or 27c, | | | | |
| | 35, 42 | | | | |

Table 97 A-F presents the preferred design solution combinations for different prior art assay situations and approaches. Table 97 G-I presents other design solutions which can be known to provide improved normalization of RT-PCR assay results and improved results, but are not the preferred design solution combinations. The other design solution combinations presented in Table 97 represent a minimum number of design solutions for obtaining improved RT-PCR normalization and results. Note that the design solution combinations presented in Table 97 represent only a few of the many possible different design solution combinations which will produce an improved RT-PCR normalization process, and RT-PCR measured and normalized assay results.

The known design solution combination associated with an RT-PCR assay determines whether the assay can be known to be associated with improved normalization of RT-PCR assay measured particular gene RN, mRNA abundance, and DGER values, and the degree to which the normalization and results can be known to be improved, relative to prior art microarray normalization practice. As discussed, prior art RT-PCR practice does not determine and normalize for pertinent UNFs. In addition prior art only rarely determines the assay values for the pertinent CNFs AE•SER and AE•AER and normalizes the assay results for them, and the common prior art RT-PCR assay practice assumptions concerning the absolute and relative PCR amplification E values and AE•AE values and AE•SE values, for compared cell sample and standard cDNAs, appear to be invalid for most prior art RT-PCR assays. Prior art RT-PCR practice does not provide the information necessary for determining the design solution combination associated with any particular assay. These factors create a situation where the design solution combination associated with a prior art RT-PCR assay is not known, and probably cannot be known retrospectively for most prior art assays. This means that, except for those few prior art RT-PCR assays which are known to be incompletely normalized for certain CNFs and/or not normalized for certain UNFs, the completeness and the validity of normalization for other prior art RT-PCR assay measured and normalized particular gene RN, mRNA abundance, and N-DGER values is unknown. Therefore absent other information, these results are essentially uninterpretable with regard to the quantitative aspects of gene expression extent, and the direction of gene regulation change. It is possible but not likely that, unknown to the prior art RT-PCR practice, a particular prior art RT-PCR assay is associated with incomplete, but improved, or even complete normalization. However, absent knowledge of the design solution combination associated with the prior art assay, it cannot be known whether the assay is associated with improved normalization or not.

The design solution combination associated with an RT-PCR assay determines the following. (i) the validity of the pertinent CNF normalization. (ii) the completeness of normalization for pertinent UNFs and CNFs. (iii) the ease of determining the assay values for the pertinent UNFs and CNFS. (iv) the ease and simplicity of the normalization process. (v) the biological accuracy of the assay measured and normalized particular gene mTN, RN, mRNA abundance, and N-DGER values. (vi) the overall interpretability of assay measured and normalized particular gene RN, mRNA abundance, and N-DGER values. (vii) the between and within RT-PCR assay intercomparability of the assay measured particular gene RN, mRNA abundance, and N-DGER values. (viii) the intercomparability of the RT-PCR assay measured cell sample particular gene RN, mRNA abundance, and N-DGER values, with cell sample particular gene RN, mRNA abundance, or N-DGER values obtained with a different microarray or non-microarray method for gene expression analysis, for which the design solution combination associated with the assay is known. It is desirable to maximize each of these characteristics as much as possible. Here, if the RT-PCR assay measured particular gene RN, mRNA abundance, or N-DGER value is biologically accurate, then; the normalization is valid and complete, the particular gene RN, mRNA abundance, or N-DGER value, can be validly interpreted, and validly intercompared with other biologically accurate RT-PCR, microarray, or other non-microarray particular gene RN, mRNA abundance, or N-DGER values. Further, such biologically accurate RT-PCR assay measured particular gene RN, mRNA abundance, or N-DGER values, can be validly used to corroborate and validate particular gene RN, mRNA abundance, or N-DGER values measured by microarray or other non-microarray assay methods.

As presented in Table 97, the only pertinent UNF or CNF which can be ignored by design for an RT-PCR assay, is the UNF PAFR. This occurs for both the preferred and other design solution combinations only when SG or random primed cDNA produced from cell sample T-RNA is used in the assay. Table 97 A, B, and G design solution combinations are associated with an RT-PCR assay which does not use standards in order to quantitate. Current prior art belief and practice indicates that accurate RT-PCR assay quantitation requires the use of one or more external or internal standards. Such belief and practice is associated with the prior art perception that such standards are necessary to control and normalize for assay AE•SE and AE•AE values which are known to vary. In principle, such standards are not needed to accurately quantitate if accurate assay values for the PAFR, SCR, AE•SER, and AE•AER, can be determined for the assay. In practice, the use of standards greatly increases the complexity of the assay since the assay values for both the standard and particular gene AE•SER and AE•AER values must be determined for the assay. In addition, it appears that the common prior art assumptions required for normalization which concern the absolute and relative standard and particular gene AE•SE and AE•AE values for compared cell sample cDNAs, are not valid. Thus, the prior art use of standards for quantitation is likely to be invalid for many prior art RT-PCR assays.

Table 97 C and D design solution combinations represent the common prior art RT-PCR practice where a standard is used and a PCR amplification Step E value (i.e. the AE•AE) is predetermined for a particular gene and its associated standard. These predetermined AE•AE values are used for normalization, and prior art assumes that each AE•AE value has the same predetermined value in different assay replicates, and for different cell samples. It seems clear that these assumptions are very frequently invalid and that the actual particular gene and standard AE•AE assay values often deviate significantly from the predetermined AE•AE values used for normalization. Note that for the Table 97 C and D design solution combinations, the normalization process is improved over the prior art normalization process because the pertinent UNFs are known to be normalized for.

If it is decided to use standards in an RT-PCR assay, including standards associated with the AHG approach, Table 97 E and F present the preferred design solution combinations for this approach. Hence, the assay particular gene and standard AE•SER and AE•AER values are determined for each separate assay, and used for normalization. Note that when multiple standards are used in the assay for quantitation, the AE•SE and AE•AE value must be determined for each separate standard.

As discussed earlier, not all assay variables which have been identified and used for normalization by prior art RT-PCR assay practice are included or considered in the Table 97 design solution combinations. Prior art appears to validly normalize for these assay variables, and it is here assumed that this is done. It is further assumed that appropriate PCR primers will be used in each assay situation.

An RT-PCR assay can be described by the design solution combination which is associated with the assay. An accurate assay design solution combination description serves as the basis for identifying the following. (i) the pertinent UNFs and CNFs which are associated with the assay. (ii) the pertinent UNFs and CNFs which can be ignored during the assay normalization process. (iii) the pertinent UNFs and CNFs assay values which must be determined and normalized for in the assay. (iv) the pertinent UNFs and CNFs which can be determined and normalized for. (v) the pertinent UNFs and CNFs which are normalized for. (vi) assumptions necessary to determine UNF and CNF assay values. Such an overall description is necessary in order to evaluate the utility, biological accuracy, and intercomparability of the assay measured particular gene comparison RN, mRNA abundance, and N-DGER values. Such an overall description should be available for every RT-PCR assay. Such an overall design solution combination description can be used to plan future RT-PCR assays, and to interpret already existing RT-PCR assay particular gene comparison mTN, RN, mRNA abundance, or NASR values. Such overall design solution combination descriptions were not created for prior art RT-PCR assays of any kind. In addition such an overall design solution combination description will allow the effective standardization of RT-PCR assay formats.

Improvement of all Gene Expression Analysis Comparison Assay Normalization Processes and Particular Gene Expression Results by Using both the A-SCR and R-SCR Assay Values for Normalization.

The determination and utility of obtaining two separate pertinent UNF and CNF normalized particular gene N-DGER values for each particular gene comparison in an assay, where one value is normalized for the A•SCR UNF assay value, and the other value is normalized for the R-SCR assay value, was discussed earlier. The A•SCR associated N-DGER value is measured in terms of the number of a particular gene's mRNA molecules per cell, while the R•SCR associated N-DGER value is measured in terms of the number of a particular gene's mRNA molecules per haploid DNA content, or haploid cell equivalent of DNA. The A•SCR and R•SCR values for particular gene comparisons in an assay may differ by a maximum of 2 fold, but could differ by more.

Determining both the A•SCR and R•SCR values for one or more particular gene comparisons in an assay provides an improved normalization process and improved particular gene comparison results. Such normalization and particular gene comparison results are improved relative to prior art particular gene comparison results, and are further improved relative to assay normalization situations where only the A•SCR or the R•SCR is normalized for. Such improvement arises from the increased ability to interpret the results and more finely define gene expression and gene expression control processes and mechanisms. In addition such further improved gene expression comparison results are more intercomparable with other gene expression comparison results.

The determination of the A•SCR and R•SCR values for each design solution combination assay described in Tables 54 through 69, 85 through 90, and 93 through 97, are further improved when both the A•SCR related and R•SCR related particular gene N-DGER values are determined for one or more particular genes in each assay. Note that unless otherwise noted SCR will refer to the A•SCR.

Improvement of SAGE Measured Cell Sample Analysis and Cell Sample Comparison Analysis Normalization Process and Assay Results by Assay Design and Measurement.

Prior art SAGE practice believes and practices that a SAGE measured particular gene mFR value for a cell sample comparison is biologically accurate, and that it accurately represents the T-DGER value for the compared particular gene mRNAs which are present in the compared cell samples. Even when such a prior art SAGE practice measured particular gene mFR value accurately represents the ratio of the particular gene mRNAs in the compared cell sample RNAs, and is therefore biologically accurate, the prior art belief and practice that the biologically correct particular gene mFR value is equal to the T-DGER value for the particular gene mRNAs in the compared cell samples, is valid only under certain restricted assay conditions. Further, it is known that the required assay conditions are often not present for a SAGE cell sample comparison. A variety of mRNA clone counting methods, including various SAGE methods, are used by the prior art to detect and quantify and compare gene expressions. Here for simplicity, the discussion will be in terms of the SAGE method, but unless otherwise noted, the discussion will apply directly to other non-SAGE clone counting methods.

In order to accurately normalize a biologically accurate SAGE measured particular gene mFR value to produce a normalized particular gene mFR value which is equal to the particular gene T-DGER value, it is necessary to determine or know, an accurate quantitative value for each prior art unconsidered normalization factor (NF) which is pertinent for the particular gene mFR value. Here, a normalized particular gene mFR value is termed a particular gene N-mFR value. A particular gene N-mFR value may be completely or incompletely normalized for all pertinent NFs.

The prior art unconsidered assay variables PAF and STM are pertinent for individual cell sample SAGE assays, while the UNFs PAFR and STMR are pertinent for cell sample comparison SAGE assays. In addition, for those cell sample comparison SAGE assays which determine the abundance value for a particular gene mRNA from the SAGE measured particular gene mF value, the unconsidered assay variable associated with the cell sample RNA isolation efficiency, or the RIE, is also pertinent. For a cell sample comparison SAGE assay, the compared cell sample RIE ratio, that is the UNF RIER, is pertinent.

As discussed earlier the determination of the assay PAF or PAFR value for even one particular gene mRNA requires a significant effort, and it is impractical to determine the PAF or PAFR value for more than a very few particular genes in an assay. The determination of the PAF and PAFR values for a particular gene (PG) was discussed earlier. The determinations of the STM and RIE assay values for a cell sample are relatively straightforward, and were also described earlier. Note that the RIE determination for a cell sample may be required for the determination of the cell sample STM value, and is incorporated into the STM and STMR values. Prior art SAGE practice does not determine or take into consideration during normalization, the assay values for STMR or PAFR. Such normalization can be done using the relationship (PG T-DGER)(PG N-mFR)=(measured PG mFR×STMR)÷(PG PAFR).

In order to obtain SAGE measured particular gene N-mFR values which can be known to be biologically accurate or improved in biological accuracy, relative to prior art SAGE practice measured particular gene mFR values which are uninterpretable with regard to biological accuracy, the following improvements in the prior art practice normalization practice are required. (i) It is necessary to use an improved normalization approach which is known to be valid, or to know that the key prior art SAGE normalization assumptions are valid, in order to determine the pertinent assay UNF values and normalize the SAGE measured particular gene mFR values for them. (ii) It is necessary to use an improved SAGE assay process for the more complete and accurate normalization of SAGE measured particular gene mFR values which includes, the identification of the pertinent UNFs for the assay, the valid and accurate determination of one or more pertinent UNF assay values, as well as the valid and accurate normalization for one or more pertinent UNF values. For SAGE assays the pertinent UNFs are the global UNFs STMR and RIER, and the non-global UNF PAFR. Because the RIER is incorporated into the STMR, improvements associated with the STMR and PAFR will be emphasized below.

It is highly likely that many, if not most, prior art SAGE assays are associated with STMR values which deviate significantly from one. Even for the SAGE comparison of the same cell sample type, the assay STMR value can deviate from one by 6 to 10 fold or more. Further, for the comparison of different cell types from the same organism, it appears that the STMR assay value can deviate from one by 10 to 20 fold or more. For a SAGE assay whose STMR value deviates from one by twofold, each SAGE measured particular gene mFR value will deviate from the particular gene T-DGER value by twofold. This assumes that the StMR effect is not compensated for by the effect of some other assay variable. It is highly likely that many prior art SAGE assays are associated with STMR values which deviate from one by significantly more than twofold.

Many prior art SAGE measured particular gene mFR values may also be associated with particular gene comparison assay PAFR values which deviate significantly form one. Such a deviation would cause the particular gene mFR value to deviate from the T-DGER value by an equal amount. The likely extent of PAFR deviation from one is likely to be less than for the STMR. The likely deviation of the PAFR from one for cell sample comparisons was discussed earlier, and is presented in Table 51.

The aggregate effect of the deviations from one of the STMR and PAFR assay values is equal to (STMR/PAFR). When the (STMR/PAFR) value equals one, then (the particular gene mFR)=(PG T-DGER). Since prior art SAGE practice does not determine the assay values for STMR or PAFR, it cannot be known for any particular prior art measured particular gene mFR value, how much it deviates from the T-DGER value for the particular gene comparison.

For a typical SAGE comparison the requirement to determine and normalize for the PAFR value for each particular gene comparison in the assay is impractical, and determining the PAFR value for even one particular gene comparison in the assay adds significantly to the complexity and effort of the assay. For the same typical SAGE assay, the requirement for determining the assay STMR value also adds significantly to the complexity and effort involved with the assay. However, because the STMR is a global assay variable, it is practical to determine the STMR value for SAGE assays which involve enough cell sample T-RNA or mRNA to determine the STM for the sample cells, or the number of cell sample T-RNA or mRNA CEs associated with the cell sample aliquot used to produce the cell sample tag library. Here, the number of T-RNA or mRNA CEs present in the cell sample aliquot used to produce the cell sample cDNA prep which is used to produce the cell sample mRNA tag library, is termed the SAGE RNA cell equivalent number or SAGE RCN. For a SAGE cell sample comparison, the ratio of the compared cell sample SAGE RCN values, is termed the SAGE RCNR. These considerations make it very desirable, if not necessary, to simplify the determination of the SAGE pertinent UNFs and CNFs as much as possible, and to eliminate the necessity for experimentally determining as many UNFs and CNFs as possible.

Earlier sections extensively discussed the underlying basis for each SAGE associated UNF. As a result of this it is possible to identify the assay factors which can and must be controlled in order to simplify the process of determining the pertinent UNF values, and normalizing for them. This knowledge makes it possible to knowingly design SAGE and other cDNA clone counting method assays which provide improved normalization processes and assay results, relative to prior art normalization processes and assay results. Further, this knowledge makes it possible to simplify and improve the improved normalization processes for such SAGE and other clone counting assay methods. These improvements can be accomplished by judicious assay design and measurement, as is discussed below.

The various design approaches which will result in an improved normalization process relative to the prior art SAGE and other clone counting methods, are presented in Table 52. The successful implementation of any one of the Table 52 design solution approaches 1-8, will produce a SAGE assay normalization process which can be known to be improved, relative to prior art normalization processes. The successful implementation of Table 52 design approach 9, will produce SAGE and other clone counting results which are known to be associated with fewer NF related false negative results than prior art SAGE and other clone counting assay results.

Prior art SAGE and other clone counting method assays are not standardized, and there are a variety of different designs practiced by the prior art. The design solutions or design components which can be used to produce improved SAGE and other clone counting assays assay normalization and assay results are presented in Table 98. Each of these design solutions or components reflects an aspect of SAGE and other clone counting method assay design which either directly or indirectly impacts on an assay pertinent NF and/or simplification of the normalization process. Different combinations of these design solutions can be used to describe an overall SAGE assay. Certain of these design solutions are discussed and further defined below.

Design Solutions 1, 2, 4, 5, 6.

If possible, the use of cell sample T-RNA is preferred. The process of isolating and characterizing T-RNA is much simpler and straightforward than the process of isolating and characterizing isolated mRNA. In addition the determination of the, cell sample T-RNA sample cell CE value, RCN value and RCNR value, the cell sample cDNA prep CE value, the value for the number of cell sample cDNA CEs, and the value for the ratio of the number of cell sample cDNA CEs, are much simpler and straightforward than for the use of cell sample mRNA. The determination of a cell sample RCN or RCNR value is much simpler and straightforward than the determination of the number and ratio of cell sample cDNA CEs used in an assay.

Design Solution 3.

The direct determination of the assay STM and STMR values was described earlier. This process is much more complex than determining the STM and STMR values indirectly by using the earlier described AHG approach.

Design Solution 7, 8.

Here, the preferred combination is the use of T-RNA and exogenous standard mRNA for the reasons discussed earlier. There are advantages to using both AHG mRNA and AHG DNA standards in the same assay.

Design Solution 9.

It is impractical to do this for more than a few particular gene mRNAs in an assay.

Design Solution 10.

Prior art methods for doing this are available.

Design Solution 11.

The determination of accurate assay values for STM and STMR, and the use of the AHG approach greatly facilitates this design solution.

TABLE 98
Design Solutions for Improving the Prior Clone Counting Method Assay
Normalization Process and the Assay Measured Particular Gene mFR Values
Assay Design Solution

| | |
|---|---|
| (1) | Use Cell Sample |
| | (a) T-RNA |
| | (b) Isolated mRNA |
| | To produce the analyzed or compared cell sample mRNA clone library. |
| (2) | Determine the Intact Cell CE Value for the |
| | (a) T-RNA |
| | (b) mRNA |
| | Of each analyzed or compared cell sample. |
| (3) | Directly Determine the Intact Cell Value for the |
| | (a) STM |
| | (b) STMR |
| | For each analyzed or compared cell sample. |
| (4) | Determine the |
| | (a) RCN value |
| | (b) RCNR value |
| | For each analyzed cell sample or compared cell samples. |
| (5) | Determine the cDNA CE value for each analyzed or compared cell sample |
| | cDNA prep which is used to produce a cell sample clone library. |
| (6) | Determine the number of cDNA CEs which are present in each analyzed or |
| | compared cell sample cDNA prep used to produce a cell sample clone library |
| | and the ratio of such number of cDNA CE values for a cell sample comparison. |
| (7) | Use one or more different Artificial Housekeeping Gene (AHG) |
| | (a) Exogenous standard mRNAs |
| | (b) Exogenous standard DNAs |
| | To determine and normalize for the assay STMR or STM value. |
| (8) | Use one or more different AHG exogenous standard mRNAs and/or DNAs to |
| | determine and normalize for the assay CNFs associated with |
| | (a) Sample statistics |
| (9) | For as many particular genes as possible in the assay determine the assay value |
| | for |
| | (a) PAF |
| | (b) PAFR |
| (10) | For each assay measured particular gene mF or mFR value, minimize as |
| | much as possible error associated with sampling statistics, sequencing, and |
| | other prior art considered assay variables. |
| (11) | For |
| | (a) Individual cell sample analysis assays |
| | (b) Cell sample comparison analysis assays |
| | Count enough tags of all kinds to minimize the occurrence of sampling |
| | statistics related false negative results. |

Relative to prior art normalization practice, the normalization of SAGE and other clone counting methods assay measured particular gene comparison mFR results is improved when one or more particular gene mFR values produced by a SAGE assay is known to be validly normalized for one or more pertinent UNFs, and/or validly normalized for one or more pertinent CNFs using AHGs. Further, overall negative assay results are improved when the particular gene abundance levels at which significant numbers of negative results occur in an assay, are known by the use of AHGs.

For a SAGE or other clone counting method, the preferred improved normalization process assay design solution combination results in: the simplified improved normalization of all particular gene mFR values in an assay for the STMR; the improved normalization of as many particular gene mFR values as possible for the PAFR; the simplified improved normalization of the CNFs associated with sample statistics; and the improved knowledge concerning the cell sample particular gene abundance levels in the assay at which sample statistics related false negative results occur to a significant extent. Other assay design solution combinations also provide lesser degrees of simplification and/or improvement of the normalization process, relative to prior art normalization processes. For simplicity, each different prior art general assay design will be discussed in terms of the Table 98 assay design solution combinations which can be known to allow the improved simplification and/or normalization of SAGE and other clone counting method produced particular gene mF and mFR values. These preferred and other improved practice design combinations are presented in Table 99.

TABLE 99
Design Solution Combinations Which Can Be Known to Provide, Relative to the
Prior Art, Improved Normalization for Pertinent UNFs and CNFs for All
SAGE or Other Clone Counting Method Measured Particular Gene mFR
Values in an Assay

| | | Pertinent NFs To Be |
|---|---|---|
| | | Determined and |
| Combination of Assay Design Solutions | | Normalized For |
| A. | Improved Design Solution Combinations for | STM |
| | Determining Particular Gene mRNA Abundance | PAF |
| | Values from the Analysis of a Cell Sample | Sampling Statistics |
| | (1) Combine Table 98 Design Solutions | Sequencing Error |
| | (a) 1a or b, 3a, 9a, 10, 11a | Other Prior Art Considered |
| B. | Preferred Improved Design Solution Combinations for | Assay Variables |
| | Determining Particular Gene mRNA Abundance | |
| | Values from the Analysis of a Cell Sample | |
| | (1) Combine Table 98 Design Solutions | |
| | (a) 1a, 2a, 4a, 7a, 8a, b, 9a, 10, 11a | |
| | or | |
| | (b) 1b, 2b, 4a, 7a, 8a, b, 9a, 10, 11a | |
| | or | |
| | (c) 1a, 2a, 5, 6, 7b, 8a, b, 9a, 10, 11a | |
| | or | |
| | (d) 1b, 2b, 5, 6, 7b, 8a, b, 9a, 10, 11a | |
| C. | Improved Design Solution Combinations for | STMR |
| | Determining Improved Particular Gene N-mFR Values | PAFR |
| | (1) Combine Table 98 Design Solutions | Sampling Statistics |
| | (a) 1a or b, 3b, 9b, 10, 11b | Sequencing Error |
| D. | Preferred Improved Design Solution Combinations for | Other Prior Art Considered |
| | Determining Improved Particular Gene N-mFR Values | Assay Variables |
| | (1) Combine Table 98 Design Solutions | |
| | (a) 1a, 2a, 4b, 7a, 8a, b, 9b, 10, 11b | |
| | or | |
| | (b) 1b, 2b, 4b, 7a, 8a, b, 9b, 10, 11b | |
| | or | |
| | (c) 1a, 2a, 5, 6, 7b, 8a, b, 9b, 10, 11b | |
| | or | |
| | (d) 1b, 2b, 5, 6, 7b, 8a, b, 9b, 10, 11b | |

Table 99 A and B describe design solution combinations for determining particular gene mF and abundance values for individual cell sample SAGE analyzes. Here, a particular gene normalized mF value or N-mF value is converted to an abundance value by using the relationship, (particular gene abundance value)=(particular gene N-mF value)(assay STM value). Table 99A involves the direct determination of the assay STM value, which is used in an improved normalization process to produce particular gene mF and abundance values which are known to be improved in biological accuracy, relative to prior art produced particular gene mF values and abundance values. The preferred design solution combinations of Table 99B describe a simplified method for determining the assay STM value as well as simplifying the determination of, and making more accurate, the values for other assay variables, by using the AHG approaches. These STM and other assay variable values are used in a simplified improved normalization process to produce particular gene mF and abundance values which are known to be improved in biological accuracy, relative to prior art SAGE produced particular gene mF and abundance values.

Table 99C involves the direct determination of the compared cell sample assay STMR value which is used in an improved normalization process to produce particular gene mFR values which are known to be improved by virtue of more accurately reflecting the particular gene T-DGER value for the cell sample comparison. The preferred design solution combinations of Table 99D describe a simplified method for determining the assay STMR value as well as simplifying the determination of and making more accurate, the assay values for other assay variables, by using the AHG approaches. These STMR and other assay variable values are used in a simplified improved normalization process to produce particular gene mFR values which are known, by virtue of more accurately reflecting the particular gene T-DGER value for the cell sample comparison, to be more accurate biologically than prior art SAGE produced particular gene mFR values.

The design solution combinations presented in Table 99 are only a few of many possible design solution combinations which can produce improved SAGE assay normalization and results.

A SAGE analysis of a cell sample or a cell sample comparison can be described by the design solution combination associated with the assay. An accurate design solution combination description serves as the basis for identifying the following. (i) The pertinent UNFs and CNFs which are associated with the assay. (ii) The pertinent UNFs and CNFs which must be determined and normalized for in the assay. (iii) The pertinent UNFs and CNFs which can be normalized for in the assay. (iv) The pertinent UNFs and CNFs which are normalized for. (v) The assumptions necessary to determine UNF and CNF assay values. Such an overall description is necessary to evaluate the utility, biological accuracy, and intercomparability, of assay measured particular gene comparison N-mFR values. Such an overall description should be available for each SAGE cell sample analysis assay. Such an overall design solution combination description can be used to plan future SAGE analyzes, and to interpret already existing SAGE produced particular gene N-mF and particular gene comparison N-mFR values. Such overall design solution combination descriptions were not created for prior art SAGE assays of any kind. In addition, such an overall design solution combination description will allow the effective standardization of SAGE assay formats.

Producing Microarray, Non-Microarray, and Clone Counting Method Improved Normalization Processes and Improved Assay Results for DGDS and DGSS mRNA Transcript Comparison Assays, and SGDS, DGDS, and DGSS RNA Transcript of any Kind Comparison Assays.

The earlier described UNFs and CNFs which are associated with microarray, non-microarray, and clone counting method SGDS mRNA transcript comparison assays, are also associated with microarray, non-microarray, and clone counting method DGDS and DGSS mRNA transcript comparison assays, and SGDS, DGDS, and DGSS RNA transcript of any kind comparison assays. As a result, improved normalization processes and improved assay results for DGDS and DGSS mRNA transcript comparison assays, and for SGDS, DGDS, and DGSS RNA transcript of any kind comparison assays are produced by: (i) identifying the pertinent assay variable associated UNFs and CNFs which are pertinent to an SGDS and/or DGDS and/or DGSS RNA transcript comparison assay; (ii) validly determining the assay value for each UNF and/or CNF which is pertinent to an SGDS and/or DGDS and/or DGSS particular gene RNA transcript comparisons assay result; (iii) utilizing the determined UNF and/or CNF assay values for normalizing each SGDS and/or DGDS and/or DGSS particular gene RNA transcript comparison in the assay for its associated pertinent UNF and/or CNF assay values. Therefore, the above described microarray, non-microarray, and clone counting method, improved assay design solution combination assays which produce improved SGDS mRNA transcript comparison assay results and improved normalization processes, also produce improved assay results and improved normalization processes for DGDS and/or DGSS mRNA transcript comparison assays, and for SGDS and/or DGDS and/or DGSS RNA transcript of any kind comparison assays. Here, RNA transcript of any kind includes one or more or all, of all types of rRNA, tRNA, mRNA, miRNA, siRNA, snoRNA, antisense RNA, and other known and unknown RNAs. In order to produce such improved RNA transcript of any kind comparison results, appropriately labeled cell sample RNA of any kind, or RNA of any kind cDNA or cRNA equivalents, must be produced for assay, and the appropriate RNA of any kind specific CDP molecules must be incorporated into the assay. Earlier described cell sample RNA or cell sample RNA cDNA or cRNA equivalents labeling methods, and labeling rationales, are adequate for labeling cell sample RNA or any kind, or cell sample RNA of any kind cDNA or cRNA equivalents. The earlier discussed methods and rationale for producing and using particular gene specific CDP molecules is also appropriate for these assays.

Certain of the improved microarray or RT-PCR SGDS mRNA transcript equivalent cDNA or cRNA comparison assay design solution combinations described in the above Tables, utilize oligo dT primer. These improved microarray or RT-PCR SGDS mRNA transcript comparison design solution combinations represent improved design solution combinations only for those SGDS, DGDS, or DGSS RNA transcript equivalent cDNA or cRNA comparison assays, which compare RNAs which possess a sufficiently long Poly A tract. Generally only eukaryotic mRNA transcripts possess such Poly A tracts, and therefore these described improved SGDS mRNA transcript comparison design solution combinations do not represent improved design solution combinations for SGDS, DGDS, or DGSS RNA transcript of any kind comparisons where the compared RNA transcripts are not associated with Poly A tracts. Further, the PAFR UNF is not pertinent to any SGDS, DGDS, or DGSS array comparison of RNA transcripts which are not associated with Poly A tracts.

The described improved microarray or RT-PCR SGDS mRNA transcript cDNA or cRNA equivalent comparison assay design solution combinations in the Tables which utilize specific gene (SG) or random primer, also represent improved microarray and RT-PCR assay design solution combinations for DGDS and DGSS mRNA transcript cDNA or cRNA equivalent comparison assays which use SG or random primer, and further represent improved microarray and RT-PCR assay improved design solution combinations for SGDS, DGDS, and DGSS, RNA transcript of any kind cDNA or cRNA equivalent comparison assay which use SG or random primer.

The described improved microarray or non-microarray SGDS mRNA transcript comparison assay design solution combinations in the Tables which directly compare mRNA transcripts, also represent improved microarray and non-microarray assay design solution combinations for DGDS and DGSS mRNA transcript comparison assays which directly compare mRNA transcripts, and further represent improved microarray and non-microarray assay design solutions for SGDS, DGDS, and DGSS RNA transcript of any kind comparison assays, which directly compare the RNA transcripts.

The described improved clone counting method SGDS mRNA transcript comparison assay design solution combinations in the Tables, also represent improved clone counting method DGDS and DGSS mRNA transcript comparison assay design solution combinations. Because the clone libraries utilized in the clone counting method assays are produced from oligo dT primed cDNA, only mRNA clones for Poly A Tract containing mRNA are present in a cell sample clone library. Therefore, the described improved clone counting method SGDS mRNA transcript comparison assay design solution combinations in the Table are not pertinent for SGDS, DGDS, or DGSS RNA transcripts of any kind assays.

A particular design solution combination described in the above Tables may produce improved assay results for an SGDS, DGDS, and DGSS RNA transcript of any kind comparison assay. Such an assay design solution combination may produce different degrees of improvement in the normalization process for: SGDS RNA comparisons relative to either DGDS or DGSS RNA comparisons; and/or DGDS RNA comparisons relative to either SGDS or DGSS RNA comparison; and/or DGSS RNA comparisons relative to either SGDS or DGDS RNA comparisons. This will be illustrated below. For this illustration the term mRNA transcript comparison also refers to the term, mRNA transcript cDNA or cRNA equivalent comparison, while the term, RNA transcript of any kind comparison also refers to the term, RNA transcript of any kind cDNA or cRNA equivalent comparison.

As an example, the improved assay design solution combination assays described in Tables 54(2a), 54(6a), 55(2a), 55(6a), 56(2a), 57(2a) 57(6a), 58(2a) 58(6a) 59(2a), and 59(6a), for SGDS mRNA transcript comparison assays, also provide improved assay results for DGDS and DGSS mRNA transcript comparison assays. Further, those improved assay design solution combination assays in Tables 54(2a) and 54(6a), 55(2a), 55(6a) and 56(2a), also produce improved assay results for SGDS, DGDS, and DGSS RNA transcripts of any kind comparison assays. For each such SGDS mRNA transcript and RNA transcript of any kind comparison assay, the assay design solution combination utilized does not require the determination of and normalization for the assay pertinent non-global UNF PL-HKR and MLDR assay values, because the assay is designed so that the PL-HKR and MLDR assay values are known to equal one for each SGDS particular gene RNA transcript comparison in the assay. Thus, for these SGDS assays the non-global PL-HKR and MLDR UNFs can be ignored for the process of normalizing each SGDS particular gene RNA transcript comparison result obtained in the assay. However, for DGDS or DGSS mRNA transcript or RNA transcript of any kind comparison assays which utilize the same improved assay design solution combinations, the PL-HKR and MLDR UNFs can be ignored for normalization only when the nucleotide complexities of the compared different particular gene undegraded RNA transcripts are the same, or nearly the same. That is, only when the nucleotide lengths of the compared different particular gene undegraded RNA transcripts are the same. For those DGDS and/or DGSS particular gene RNA transcript comparisons in any assay, where the compared different particular gene undegraded RNA transcripts differ significantly in nucleotide complexity or nucleotide length, the assay PL-HKR and MLDR values must be determined and used for normalization of the particular gene RNA transcript comparison assay result.

For each of the SGDS assay design solution combinations identified above for SGDS mRNA transcript comparison assays, the assay design solution also does not require the determination of and the normalization for the non-global UNF PS-HKR. Here, each improved SGDS mRNA transcript comparison assay is designed so that the PS-HKR assay value is known to equal one for each SGDS particular gene mRNA transcript comparison in the assay. For each of these assay design solution combination assays, the PS-HKR assay value also equals one for an SGDS RNA transcript of any kind comparison assay. This occurs because for these assays the SGDS compared RNA transcripts have essentially the same nucleotide lengths and nucleotide sequences. However, for DGDS and DGSS mRNA transcript or RNA transcript of any kind comparisons which utilize the same improved assay design solution combination assays, the non-global UNF PS-HKR assay value cannot be known to be equal to one for each particular gene RNA transcript comparison in the assay. This occurs because the nucleotide sequences for the DGDS and DGSS compared particular gene RNA transcript nucleotide sequences are not the same. Thus, these improved DGDS and DGSS RNA transcript comparison assays the assay PS-HKR value for each particular gene RNA transcript comparison in the assay must be determined and used for normalization of the particular gene comparison assay result.

For these above identified improved assay design solution combination assays, the degree of improvement for the assay normalization process for the non-global UNFs MLDR, PL-HKR, and PS-HKR, is greater for SGDS assays than for DGDS and DGSS assays which use the same assay design solution combinations.

For these same above identified improved assay design solution combinations for SGDS mRNA transcript comparison assays, as well as for DGDS mRNA transcript comparison assays and SGDS and DGDS RNA transcript of any kind comparison assays, it is necessary to determine the assay value for the global UNF SCR. However, for DGSS mRNA transcript or RNA transcript of any kind comparison assays which utilized the same identified assay design solution combinations, the global UNF SCR assay value is known to in effect equal one, and therefore the SCR can be ignored for the normalization of DGSS particular gene mRNA transcript or RNA transcript of any kind comparison assay results. This occurs because the different particular gene RNA transcript LPNs which are compared in a DGSS assay are present in the same cell sample LPN prep for microarray assays. Thus, for a microarray DGSS assay each compared particular gene RNA transcript LPN prep which is present in the bulk cell sample LPN prep, is associated with the same number of cell sample LPN cell equivalents. This assumes the validity of the R and F mole assumptions. For non-microarray DGSS assays, depending on how the assay is designed, it may or may not be necessary to determine the SCR value associated with a DGSS particular gene RNA transcript LPN comparison assay. This is especially true for RT-PCR DGSS particular gene RNA transcript cDNA comparisons. For these above identified improved assay design solution combination assays, the degree of improvement for the assay normalization process for the global UNF SCR assay value is greater for DGSS RNA transcript comparison assays, than for SGDS or DGDS assays which utilize the same improved assay design solution combinations.

As a further example, the improved assay design solution combination assays described in Tables 54(9a-16a), 55(9a-16a), 57(9a-16a), 58(14a-21a), 59(9a-16a), for SGDS mRNA transcript comparison assays, also provide improved assay results and improved assay normalization processes for DGDS and DGSS mRNA transcript comparison assays, and SGDS, DGDS, and DGSS RNA transcript of any kind comparison assays. For such identified improved assay design solution combination SGDS, and DGDS mRNA transcript, and RNA transcript of all kind comparison assays, the assay value for the global UNF LLSR must be determined and used for the normalization of each SGDS or DGDS particular gene RNA transcript comparison in the assay. However, for DGSS mRNA transcript or RNA transcript of any kind comparison assays which utilize the same identified improved assay design solution combinations, the DGSS assay LLSR value is effectively equal to one for all DGSS particular gene RNA transcript comparisons in the assay, and can be ignored for the normalization process for each DGSS particular gene RNA transcript comparison assay result. Here, with regard to the LLSR UNF, the degree of improvement of the normalization process for DGSS assays, is greater than that for the SGDS or DGDS assays.

As another example, the improved assay design solution combination assays described in Tables 29(4), 30(4), 31(4), 32(4), 33(4), 34(4), and 35(4), for SGDS mRNA transcript comparison assays, also provide improved assay results and improved assay normalization processes for DGDS and SGDS mRNA transcript comparison assays and SGDS, DGDS, and DGSS RNA transcript of any kind comparison assays. For such identified improved assay design solution combination SGDS mRNA transcript or RNA transcript of any kind comparison assays, the assay value for the non-global UNF SBNR is known to equal one for each SGDS particular gene mRNA transcript or RNA transcript of any kind comparison assay, and therefore the UNF SBNR can be ignored for the process of normalization of each SGDS particular gene RNA transcript comparison in the assay. This occurs because the SGDS compared RNA transcript LPN molecules are known to be labeled with the same indirect ligand label and are known to have essentially the same nucleotide lengths and nucleotide sequences, and label ligand densities. However, for DGDS and DGSS mRNA transcript and RNA transcript of any kind comparison assays using these same improved assay design solution combinations, the assay SBNR value for each DGDS or DGSS particular gene RNA transcript comparison in the assay, cannot be known to equal one. This occurs because for each DGDS or DGSS particular gene RNA transcript comparison in the assay, the nucleotide sequences of the compared RNA transcripts are different, and the nucleotide lengths and the label ligand densities associated with the compared RNA transcripts can be significantly different. As a result, for each DGDS or DGSS particular gene RNA transcript comparison assay result, the associated SBNR assay value must be determined and used to normalize the assay result. Here, with regard to the SBNR UNF, the degree of improvement of the normalization process for SGDS assays is greater than that for DGDS or DGSS assays.

As an additional example, the improved clone counting method SGDS mRNA transcript comparison assay design solution combinations described in Table 99C and D, also provide improved assay results and improved assay normalization processes for DGDS and DGSS mRNA transcript comparison assays, and SGDS, DGDS, and DGSS RNA transcript of any kind comparison assays. For such DGSS mRNA transcript comparison assays the assay value for the STMR UNF is known to equal one. This occurs because the DGSS compared particular gene clones are present in the same cell sample mRNA transcript clone library. For such SGDS and DGDS mRNA transcript comparisons, the assay STMR value must be determined and normalized for.

Prior art microarray practice SGDS mRNA transcript comparison assays do not determine the assay values for or normalize for, assay pertinent global and non-global UNFs. Further, the prior art determination for and normalization for assay pertinent global and non-global CNFs cannot be known to be valid. For such SGDS microarray assays, as many as fourteen NFs may be pertinent to these assay, and eight of these are UNFs. One prior art microarray SGDS mRNA transcript type 1 LPN comparison assay may be associated with thirteen NFs, and seven of these are the global UNF SCR, and the non-global UNFs PAFR, MLDR, PL-HKR, PS-HKR, PSAR, and PSSR. For such an SGDS comparison the number of possible particular gene comparisons is equal to the number of genes being compared. As an example, for a microarray mRNA transcript comparison of 100 different particular genes, the total number of SGDS particular gene comparisons in the assay is 100. For this assay, the pertinent global UNF SCR is associated with only one assay value, and the assay SCR value is the same for all 100 SGDS particular gene comparisons in the assay. For this assay multiple different assay values for one non-global UNF may be, and very often are, associated with the SGDS comparison assay. As an example, in the same assay one subset of SGDS particular gene mRNA transcript comparisons can be associated with one assay value for the non-global UNF PSAR, while one or more different subsets of particular gene mRNA transcript comparisons in the same assay are associated with PSAR assay values which differ significantly from every other subset of particular gene mRNA transcript comparisons in the same assay. For valid normalization of the assay results for the non-global UNF, it is necessary to know or determine the assay value for the non-global UNF which is associated with each different particular gene mRNA transcript comparison assay result in the assay.

As discussed earlier, the global UNF SCR, and non-global UNFs PAFR, MLDR, PL-HKR, PS-HKR, PSAR, and PSSR, may also be pertinent for microarray DGDS or DGSS particular gene mRNA transcript type 1 LPN comparison assays. For such DGDS and DGSS comparisons, the number of potential particular gene mRNA transcript comparisons is very much larger than for an SGDS comparison. For a DGDS or DGSS comparison assay, the number of possible different gene comparisons is equal to X²−X, where X represents the number of genes being compared in the assay. For a microarray DGDS or DGSS comparison of 100 different particular gene mRNA transcripts, X=100, and (X²−X)=9900 different possible different particular gene comparisons. For these 100 different particular genes, for a microarray assay which determined a particular gene mRNA transcript comparison assay result for every possible SGDS, DGDS, and DGSS particular gene mRNA transcript comparison in the assay, the total number of particular gene mRNA transcript comparison assay results would be equal to about 19,900. Microarrays exist which allow the comparison of the expression of about 4300 different E. coli particular gene mRNA transcripts, and about 30,000 different human particular gene mRNA transcripts. For a microarray mRNA transcript comparison assay the total number of different possible SGDS, DGDS, and DGSS particular gene mRNA transcript comparisons is about 3.6×10⁷ for E. coli, and about 1.8×10⁹ for human. These numbers relate only to mRNA transcript comparisons and do not include other RNA transcript comparisons A complete description of the particular gene expression extent relationships associated with a cell sample mRNA transcript comparison requires, at a minimum, gene expression information concerning all of the SGDS and DGDS and DGSS particular gene mRNA transcript comparisons in the cell sample comparison assay. In addition, understanding the interactions between the compared mRNA transcripts in the cells would require further information on the gene expression extent relationships between each particular gene mRNA transcript in a cell and other non-messenger RNAs in the cell, such as siRNAs, miRNAs, snoRNAs, antisense RNAs, rRNAs, tRNAs, and other known or unknown RNAs in the cell. As a result of focusing only on SGDS particular gene mRNA transcript comparisons almost exclusively, prior art obtains and takes into consideration only a very small fraction of the particular gene mRNA transcript expression comparison information for a cell sample and an even smaller fraction of the mRNA vs mRNA and mRNA vs other RNA comparisons which exist for a cell sample comparison. As an example, the E. coli and human SGDS mRNA transcript comparisons for an assay represent respectively only about 0.0001 and 0.00003 of the total number of SGDS and DGDS and DGSS particular gene mRNA transcript comparisons which are associated with these cell sample comparison assays.

Generally, but not always, a particular non-global UNF is more likely to deviate from the assay value of one for a microarray assay DGDS or DGSS particular gene mRNA transcript comparison, than an SGDS comparison. This occurs because DGDS and DGSS comparisons always involve a comparison of mRNA transcripts with different nucleotide sequences, and often involve comparisons of mRNA transcripts with different nucleotide lengths. Further, for the DGDS and DGSS comparison of the same different particular gene mRNA transcripts in one assay, the DGDS comparison assay value for a particular non-global UNF often differs from the DGSS comparison assay value for the same UNF. The overall situation with regard to the determination of and normalization for global and non-global UNFs is much more complex for a microarray assay which is concerned with SGDS and DGDS and DGSS particular gene mRNA transcript comparison assay results. Note that for simplicity the above discussion focused on particular gene mRNA transcript comparisons, but the discussion is also directly applicable to particular gene RNA transcript of any kind comparisons.

Most SGDS, DGDS, and DGSS particular gene mRNA transcript comparison assay results produced by prior art microarray cell sample gene expression comparison assays are associated with one or more assay pertinent global or non-global UNFs whose assay values deviate significantly from one. Prior art microarray measured particular gene mRNA transcript comparison assay results are not normalized for these UNF assay values. As discussed, such UNF deviations from one can cause the assay measured particular gene comparison assay result to deviate from biological accuracy. Such deviations are relevant only if the magnitude of the deviation is significant, relative to the microarray assay measurement accuracy. The measurement accuracy of prior art microarray assays is commonly claimed to be within ±1.2 fold to ±2 fold. Table 51 presents what are believed to be conservative estimates for the magnitude of deviation of UNFs and CNF assay values from one which are commonly associated with a typical microarray SGDS mRNA transcript comparison assay. These magnitudes of deviation from one for the UNFs and CNFs generally reflect conservative estimated deviations which would also be associated with prior art microarray DGDS and DGSS mRNA transcript comparison assays. An exception is the DGSS assay value for SCR, which will equal one for most such DGSS assays. In addition, it is likely that the estimated commonly occurring non-global UNF MLDR, PL-HKR, PS-HKR, PSAR, and PSSR, assay vales associated with DGDS and DGSS comparisons, are significantly larger than the estimated commonly occurring values for the same UNFs in an SGDS comparison. The estimated DGDS and DGSS assay values for these non-global UNFs are respectively, MLDR=3-5 fold, PL-HKR=2-3 fold, PS-HKR=2-3 fold, PSAR=2-4 fold, PSSR=2-3 fold.

In the context of the assay measurement accuracy claimed for a typical prior art microarray assay, the deviation of even one of the UNFs from one is large enough to significantly affect the quantitative value, interpretation, and biological accuracy of a microarray assay measured SGDS, DGDS, or DGSS, particular gene mRNA transcript comparison assay results. The effect of such UNF deviations from one on the quantitative value, the interpretation, and the biological accuracy of SGDS particular gene comparison assay results were discussed earlier. The normalization of such SGDS particular gene comparison assay results for the UNF deviations from one, was also discussed. Both discussions apply directly to DGDS and DGSS mRNA transcript and RNA transcript of any kind comparison assay results.

Prior art microarray and non-microarray practice does not identify or determine the pertinent UNFs which are associated with the SGDS, DGDS, or DGSS, particular gene mRNA transcript, or RNA transcript of any kind, comparisons in the prior art assay. As a result it cannot be known whether a prior art produced SGDS, DGDS, or DGSS particular gene RNA transcript comparison RASR value requires normalization for the UNFs or not. Therefore, in order to determine whether a prior art produced SGDS, DGDS, or DGSS, particular gene RNA transcript comparison RASR value requires normalization for the UNFs, the following steps are necessary. (i) identify the UNFs which are pertinent to each SGDS and/or DGDS and/or DGSS particular gene mRNA transcript comparison assay result. (ii) then determine a quantitative measure of the assay value for each pertinent SGDS UNF, and/or each pertinent DGDS UNF, and/or each pertinent DGSS UNF, in order to determine whether normalization is necessary for each pertinent UNF. The determinations of the assay value of and the normalization process for, each different UNF were described earlier in the context of SGDS particular gene mRNA transcript comparisons, and these descriptions apply directly to all, SGDS and DGDS and DGSS mRNA transcript comparison and RNA transcript of any kind comparison, assay associated UNFs. If normalization is required for an SGDS, DGDS, or DGDS particular gene RNA transcript comparison RASR value, the measured UNF assay values are utilized in the normalization process to accomplish the normalization. The normalization process then produces an improved SGDS, or DGDS or DGSS particular gene RNA transcript comparison assay result. For a typical microarray or non-microarray assay, the requirement to identify, determine the assay value for, and normalize for, the pertinent UNFs for the SGDS particular gene mRNA transcript comparisons, adds a very significant amount of complexity and effort to the microarray and non-microarray assay, relative to a prior art microarray or non-microarray gene expression comparison assay. For a typical microarray or non-microarray assay, the requirement to identify, determine the assay value for, and normalize for, the pertinent UNFs for either or both DGDS or DGSS particular gene mRNA transcript comparisons, adds an extremely large amount of complexity and effort to the microarray and non-microarray assay, relative to doing this same process for only SGDS particular gene mRNA transcript comparisons. Clearly, a microarray or non-microarray assay which does this same UNF related process for SGDS, DGDS, and DGSS particular gene RNA transcript of all kinds comparisons, including mRNA transcript comparisons, would add even more complexity and effort to the assay. Further, as discussed earlier, it is not practical to determine the PAFR or PSSR UNF assay values for more than a very few SGDS particular gene mRNA transcript comparisons in an assay. This is also the case for DGDS and DGSS particular gene mRNA transcript and RNA transcript of any kind comparisons. As also discussed earlier, it is often not feasible to determine the assay values for the UNFs PL-HKR and PS-HKR for SGDS particular gene mRNA transcript comparisons, and this is also true for DGDS and DGSS particular gene mRNA transcript and RNA transcripts of any kind comparisons.

The valid determination of assay values for pertinent CNFs also adds complexity and effort to a microarray assay. The use of the earlier described improved method for determining the assay values for, and normalizing the SGDS mRNA transcript comparison assay results for, the CNFs spatial, print tip, print plate, intensity and scale, can also be used for determining the assay values for, and normalizing the DGDS and DGSS mRNA transcript and RNA transcript of any kind comparison assay results for the pertinent CNFs. Such use will also add a very large amount of complexity and effort to the microarray and non-microarray assays.

The above described considerations make it very desirable, if not necessary, to simplify the determination of pertinent CNF and UNF assay values and the normalization process as much as possible, and to eliminate the necessity for experimentally determining the assay values for as many CNFs and UNFs as possible. Here it is particularly desirable to eliminate the need to determine the assay values for those UNFs or CNFs which cannot be determined, such as the UNFs PAFR and PSSR, and those which are currently difficult to determine, such as PL-HKR and PS-HKR. Earlier sections extensively discussed the underlying basis for each microarray and non-microarray assay UNF, and the assay situations under which each UNF is pertinent. These earlier discussions focused primarily on SGDS mRNA transcript comparisons but are directly applicable to SGDS, DGDS, and DGSS mRNA transcript and RNA transcript of any kind comparisons. As a result of these earlier discussions, it is possible to identify the assay factors which can and must be controlled for different microarray and non-microarray SGDS, DGDS, and DGSS mRNA transcript and RNA transcript of any kind comparison assay situations, in order to accurately normalize assay results, and to simplify the process of determining the assay values for pertinent UNFs and CNFs which are associated with SGDS and/or DGDS, and/or DGSS RNA transcript comparison assays, and normalizing for them. This knowledge make it possible to knowingly design microarray and non-microarray SGDS, DGDS, and DGSS particular gene mRNA transcript and RNA transcript of any kind comparison assays which do not require the direct determination of certain UNF and CNF assay values, including PAFR, PL-HKR, and PSSR, in order to validly normalize for these UNFs or CNFs. The overall result of such assay design solutions is a simplified version of the improved microarray and non-microarray normalization process. This can be accomplished by judicious assay design and experimental measurement, as is discussed below.

The various microarray and non-microarray and clone counting method SGDS, DGDS, and DGSS, particular gene mRNA transcript and RNA transcript of any kind comparison assay design approaches which will result in an improved normalization process and improved particular gene RNA transcript comparison assay results for these assays, relative to the prior art microarray and non-microarray RNA transcript comparison assay normalization processes and RNA transcript comparison assay results, are presented in Table 52. The successful implementation of any one of the Table 52 design approaches 1-8 will produce a microarray of non-microarray SGDS, DGDS, or DGSS RNA transcript comparison assay normalization process and a particular gene RNA transcript comparison assay result or results which can be known to be improved, relative to prior art microarray and non-microarray assay normalization processes and particular gene RNA transcript comparison results. The successful implementation of Table 52 design approach 9, will produce microarray and non-microarray SGDS, DGDS, and DGSS particular gene RNA transcript comparison assay results which are known to be associated with fewer CNF and UNF related false negative results than prior art microarray and non-microarray assay results.

Prior art microarray and non-microarray design is not standardized, and there are a variety of different microarray and non-microarray assay designs practiced by the prior art. The improvement of the normalization process for each of these prior art practice assay designs has been discussed earlier in the context of SGDS particular gene mRNA transcript comparisons. These discussions also apply directly to microarray and non-microarray DGDS and DGSS particular gene mRNA transcript comparison assays, as well as microarray and non-microarray SGDS, DGDS, and DGSS particular gene RNA transcript of any kind comparison assays, except for the earlier discussed minor exceptions.

The design solutions or design components used to produce the earlier discussed improved microarray SGDS mRNA transcript direct label LPN comparison assay design solution combinations described in Tables 54 through 69, are presented in Table 53. Even though the Tables 54 through 69 design solution combinations are designed specifically to produce improved SGDS particular gene mRNA transcript comparison assay results, each of these Table 54 through 69 design solution combinations will also provide improved DGDS and DGSS particular gene mRNA transcript comparison assay results as well as improved SGDS, DGDS, and DGSS particular gene RNA of any kind comparison assay results, except for the few earlier noted exceptions. As discussed earlier however, the degree of improvement of the assay results may be less for the DGDS and DGSS mRNA transcript comparison assays and the SGDS, DGDS, and DGSS RNA transcript of any kind comparison assays, relative to the SGDS mRNA transcript comparison assays. Part of the reason for this is that for the design of the SGDS mRNA comparison assays the range of specification for a particular design solution, may be different than the range of specification for the same Table 53 design solution used for a DGDS or DGSS mRNA transcript comparison assay. As an example, for Table 54 design solution combination assay (5a), Table 53 design solutions are specified. In this SGDS mRNA transcript LPN comparison context, design solution 18 specifies that for each SGDS particular gene mRNA transcript in the assay, the compared LPN nucleotide lengths are the same, and design solution 19 specifies that the nucleotide lengths and nucleotide sequences of compared particular gene mRNA transcripts are the same. For this SGDS comparison assay design solution combination, the specification of design solution 18 may or may not be met for a DGDS or DGSS particular gene comparison, and the design solution 19 design solution specification cannot be met. For this Table 54(5a) design solution combination, for SGDS comparisons the MLDR, PL-HKR, and PS-HKR UNFs can be ignored for the normalization of each particular gene mRNA transcript comparison assay result, because it is known that their assay values equal one or nearly one. However, for DGDS and DGSS mRNA transcript comparison assays using this Table 54(5a) design solution combination, the MLDR, PL-HKR, and PS-HKR UNF assay values associated with each particular gene mRNA transcript comparison assay result cannot be known to equal one. Because of this, for DGDS and DGSS comparison assays, the MLDR, P-HKR, PS-HKR UNF assay values must be determined for each particular gene mRNA transcript comparison assay result, and normalized for. Further, for SGDS and DGDS mRNA transcript comparisons using the Table 54(5a) assay design solution combination, the pertinent UNFs PSSR and PAFR can be known to equal one and are therefore ignorable for normalization, and the SCR UNF assay value is determined and normalized for. Thus, when used for DGDS and/or DGSS RNA transcript comparisons, the Table 54(5a) assay design solution combination provides improved normalization for all pertinent UNFs, except PS-HKR. As discussed the information necessary to obtain the PS-HKR assay value is not currently known, but can be determined. When used for SGDS RNA transcript comparisons, the Table 54(5a) assay design solution combination provides improved normalization for all pertinent UNFs, including PS-HKR. Overall then, the Table 54(5a) assay design solution combination provides improved normalization and improved particular gene RNA transcript comparison assay results for SGDS and/or DGDS and/or DGSS RNA transcript comparisons. However, the degree of improvement of the normalization process and improvement of the assay results is greater for SGDS RNA transcript comparisons using the Table 54(5a) assay design solution combination, than for DGDS and/or DGSS RNA transcript comparisons using the same Table 54(5a) assay design solution combination. This illustrates that the interpretation of a design solution condition specification and its effect on the improvement of the normalization process, should be made in the context of the intended use of the assay, that is whether an SGDS or DGDS or DGSS RNA transcript comparison is being done.

Relative to prior art normalization practice, the normalization of microarray SGDS, DGDS, and DGSS particular gene mRNA transcript comparison assay results, or RNA transcript of any kind comparison assay results, is improved when one or more SGDS and/or DGDS and/or DGSS particular gene RNA transcript comparison assay measured RASR values is known to be validly normalized for one or more of the following. (i) one or more pertinent CNFs. (ii) one or more pertinent UNFs. (iii) one or more pertinent UNFs and one or more pertinent CNFs. (iv) one or more pertinent UNFs and all pertinent CNFs. (v) all pertinent CNFs. (vi) all pertinent UNFs. (vii) all pertinent UNFs and all pertinent CNFs.

For a microarray or non-microarray SGDS mRNA transcript comparison assay, or RNA transcript of any kind comparison assay, a preferred improved assay design solution combination results in the valid normalization of all SGDS particular gene mRNA transcript comparison assay results, or all SGDS particular gene RNA transcript of any kind comparison assay results, in the assay, for all pertinent UNFs and CNFs, and also results in minimizing the number of UNF and CNF related false negative results which are associated with the SGDS RNA transcript comparisons in the assay. Such assay design solution combinations were presented in Tables 54-99 and discussed earlier.

For a microarray or non-microarray DGDS or DGSS, mRNA transcript comparison assay, or RNA transcript of any kind comparison assay, a preferred improved assay design solution combination results in the valid normalization of all DGDS or DGSS particular gene mRNA transcript comparison assay results, or all DGDS or DGSS particular gene RNA of any kind comparison assay results in the assay, for all pertinent UNFs and CNFs, and also results in minimizing the number of UNF and CNF related false negative results which are associated with the DDS or DGSS RNA transcript comparisons in the assay.

Similarly, for a microarray or non-microarray combined SGDS, DGDS, and DGSS, mRNA transcript comparison assay, or RNA transcript of any kind comparison assay, a preferred improved assay design solution combination results in the valid normalization of all SGDS, DGDS, and DGSS, mRNA transcript comparisons assay results, or RNA transcript of any kind comparison assay results, in the assay for all pertinent UNFs and CNFs, and also results in minimizing the number of UNF and CNF related false negative results which are associated with the SGDS and DGDS and DGSS RNA transcript comparisons in the assay.

As earlier discussed, a variety of different microarray and different non-microarray assay designs are practiced by the prior art, and different assay designs can be associated with different combinations for pertinent UNFs and CNFs. This was extensively discussed earlier for SGDS microarray and non-microarray assays, and discussed above for DGDS and DGSS microarray assays. For SGDS, DGDS and DGSS RNA transcript comparison assays, certain of these prior art general assay designs are associated with pertinent UNFs, such as PSSR and PAFR, whose assay values can practically be determined for only a very few particular genes in an assay, or with pertinent UNFs such as the PL-HKR and PS-HKR, whose assay values cannot currently be determined due to lack of hybridization kinetic information which is currently unknown, but is attainable by experimentation. Therefore, some prior art general assay designs cannot be modified to allow the improved normalization for all pertinent UNFs and CNFs. This was extensively discussed earlier for microarray and non-microarray SGDS mRNA transcript comparison assays. This earlier discussion is directly applicable to microarray and non-microarray DGDS and DGSS RNA transcript comparison assays, except for the above discussed differences. One difference involves the UNF PS-HKR. For SGDS RNA transcript comparison assays, the assay can be designed so that the PS-HKR associated with each SGDS particular gene mRNA transcript comparison assay, or RNA transcript of any kind assay comparison assay, can be known to have an assay value of essentially one. Here, for each particular gene comparison in the assay, the PS-HKR assay value is equal to one, and can therefore be ignored for normalization. In contrast, DGDS and/or DGSS RNA transcript comparison assays cannot be designed so that it is known that the assay PS-HKR associated with each particular gene RNA transcript comparison in the assay has an assay value of one. DGDS and/or DGSS comparison assays can be designed so that many, if not most, particular gene RNA transcript comparison associated PS-HKR assay values equal one or nearly one, while the PS-HKR assay values for other particular gene RNA transcript comparisons do not equal one. Here, the assay PS-HKR value associated with particular gene RNA transcript comparisons in the assay must be determined and then used in the assay normalization process. Currently the information necessary to determine the PS-HKR assay value is not available, but can be determined by experimentation. Note that SGDS, DGDS, and DGSS, RNA transcript comparison assays can be designed so that the assay values for PL-HKR, PSSR, PAFR which are associated with each SGDS or DGDS or DGSS particular gene RNA transcript comparison in one assay, are associated with assay values of one for the assay. This is illustrated in Table 100.

Microarray assay design solution combinations which can be known to provide improved normalization for all SGDS and/or DGDS and/or DGSS, particular gene mRNA transcript comparisons in an assay or RNA transcript of any kind comparison assay, or RNA transcripts of all kinds comparison assay, are described in Tables 100 through 102. These assay design solution combinations are the currently preferred microarray assay design solution combinations for these general assay designs. Each of these Table 100 through 102 described assay design solution combinations provides improved normalization for all pertinent UNFs which are associated with all SGDS particular gene RNA transcript comparisons in the assay. However, each of these Table 100 through 102 described assay design solution combinations, provides improved normalization for all DGDS and DGSS particular gene RNA transcript comparisons in an assay for all pertinent UNFs except PS-HKR.

TABLE 100
Preferred Design Solution Combinations Which Can Be Known to Completely Normalize
All or Essentially All, Microarray Assay SGDS, DGDS, and DGSS Particular Gene
RASR Values for All Pertinent UNFs and CNFs Comparison of SG Primed LPNs
Produced from T-RNA

| | | NFs Which Can Be Ignored | | | Pertinent NFs to Be Determined | | |
|---|---|---|---|---|---|---|---|
| Combination of | | For Normalization | | | and Normalized For | | |
| Assay Design | | SGDS | DGDS | DGSS | SGDS | DGDS | DGSS |
| Solutions | | Comparison | Comparison | Comparison | Comparison | Comparison | Comparison |
| | Comparison of Type | PAFR | PAFR | SCR | SCR | SCR | PSAR |
| | 1 Directly Labeled | MLDR | MLDR | PAFR | PSAR | PSAR | PS-HKR |
| | LPNs Produced | PL-HKR | PL-HKR | MLDR | Spatial | PS-HKR | Spatial |
| | from Cell Sample | PS-HKR | PSSR | PL-HKR | Print Tip | Spatial | Print Tip |
| | T-RNA | PSSR | LLSR | PSSR | Print Plate | Print Tip | Print Plate |
| (1) | Combine | LLSR | C-HKR | LLSR | Intensity | Print Plate | Intensity |
| | Table 53 Design | C-HKR | | C-HKR | Scale | Intensity | Scale |
| | Solutions 2, 4a | | | | | Scale | |
| | or b, 5a, 6, 8a, c, | | | | | | |
| | 10a, 13b, 14, | | | | | | |
| | 15a, 16a, 18a, 34 | | | | | | |
| | Comparison of Type | PAFR | PAFR | SCR | SCR | SCR | PS-HKR |
| | 2 Directly Labeled | MLDR | MLDR | LLSR | LLSR | LLSR | Spatial |
| | LPNs Produced | PL-HKR | PL-HKR | PAFR | Spatial | PS-HKR | Print Tip |
| | from Cell Sample | PS-HKR | PSAR | MLDR | Print Tip | Spatial | Print Plate |
| | T-RNA | PSAR | PSSR | PL-HKR | Print Plate | Print Tip | Intensity |
| (2) | Combine | PSSR | C-HKR | PSAR | Intensity | Print Plate | Scale |
| | Table 53 Design | C-HKR | | PSSR | Scale | Intensity | |
| | Solutions 2, 4a | | | C-HKR | | Scale | |
| | or b, 5b, 6, 8a, b, | | | | | | |
| | 10 a, 14, 15a, | | | | | | |
| | 16a, 18a, 34 | | | | | | |
| | Comparison of Type | PAFR | PAFR | SCR | SCR | SCR | SSAR |
| | 1 Indirect Labeled | MLDR | MLDR | C-HKR | C-HKR | C-HKR | PS-HKR |
| | LPNs Produced | PL-HKR | PL-HKR | PAFR | SSAR | SSAR | Spatial |
| | from Cell Sample | PS-HKR | SBNR | MLDR | Spatial | PS-HKR | Print Tip |
| | T-RNA | SBNR | LLSR | PL-HKR | Print Tip | Spatial | Print Plate |
| (3) | Combine | LLSR | | SBNR | Print Plate | Print Tip | Intensity |
| | Table 74 Design | | | LLSR | Intensity | Print Plate | Scale |
| | Solutions | | | | Scale | Intensity | |
| | (a) 2, 4a or b, | | | | | Scale | |
| | 5a, 6, 8a, d, | | | | | | |
| | 10a, 13a, | | | | | | |
| | 14, 15a, | | | | | | |
| | 16a, 18a, | | | | | | |
| | 34, 35a | | | | | | |
| | Comparison of Type | PAFR | PAFR | SCR | SCR | SCR | PS-HKR |
| | 2 Indirect Labeled | MLDR | MLDR | C-HKR | C-HKR | C-HKR | Spatial |
| | LPNs Produced | PL-HKR | PL-HKR | PAFR | LLSR | LLSR | Print Tip |
| | from Cell Sample | PS-HKR | SBNR | MLDR | Spatial | PS-HKR | Print Plate |
| | T-RNA | SBNR | SSAR | PL-HKR | Print Tip | Spatial | Intensity |
| (4) | Combine | SSAR | | SBNR | Print Plate | Print Tip | Scale |
| | Table 74 Design | | | SSAR | Intensity | Print Plate | |
| | Solutions | | | LLSR | Scale | Intensity | |
| | (a) 2, 4a or b, | | | | | Scale | |
| | 5b, 6, 8a, | | | | | | |
| | d, 10a, | | | | | | |
| | 13a, 14, | | | | | | |
| | 15a, 16a, | | | | | | |
| | 18a, 34, 35 | | | | | | |

TABLE 101
Preferred Design Solution Combinations Which Can Be Known to Completely Normalize All or
Some Microarray Assay SGDS, DGDS, and DGSS Particular Gene RASR Values for All or
Some Pertinent UNFs and CNFs. Comparison of Random Primed LPNs Produced from T-RNA

| | | NFs Which Can Be Ignored | | | Pertinent NFs to Be Determined | | |
|---|---|---|---|---|---|---|---|
| Combination of | | For Normalization | | | and Normalized For | | |
| Assay Design | | SGDS | DGDS | DGSS | SGDS | DGDS | DGSS |
| Solutions | | Comparison | Comparison | Comparison | Comparison | Comparison | Comparison |
| | Comparison of | PAFR | PAFR | SCR | SCR | SCR | PSAR |
| | Type 1 Directly | MLDR | MLDR | PAFR | PSAR | PSAR | PS-HKR |
| | Labeled LPNs | PL-HKR | PL-HKR | MLDR | Spatial | PS-HKR | Spatial |
| | Produced from Cell | PS-HKR | PSSR | PL-HKR | Print Tip | Spatial | Print Tip |
| | Sample T-RNA | PSSR | LLSR | PSSR | Print Plate | Print Tip | Print Plate |
| (1) | Combine | LLSR | C-HKR | LLSR | Intensity | Print Plate | Intensity |
| | Table 53 Design | C-HKR | | C-HKR | Scale | Intensity | Scale |
| | Solutions | | | | | Scale | |
| | (a) 2, 4a or b, 5a, | | | | | | |
| | 6, 8a, c, 11, 13b, | | | | | | |
| | 14, 15a, 16a, 18a, | | | | | | |
| | 34 | | | | | | |
| | Comparison of | PAFR | PAFR | SCR | SCR | SCR | SSAR |
| | Type 1 Indirect | MLDR | MLDR | C-HKR | C-HKR | C-HKR | PS-HKR |
| | Label LPNs | PL-HKR | PL-HKR | PAFR | SSAR | SSAR | Spatial |
| | Produced from Cell | PS-HKR | SBNR | MLDR | Spatial | PS-HKR | Print Tip |
| | Sample T-RNA | SBNR | LLSR | PL-HKR | Print Tip | Spatial | Print Plate |
| (2) | Combine | LLSR | | SBNR | Print Plate | Print Tip | Intensity |
| | Table 74 Design | | | LLSR | Intensity | Print Plate | Scale |
| | Solutions | | | | Scale | Intensity | |
| | (a) 2, 4a or b, 5a, | | | | | Scale | |
| | 6, 8a, d, 11, 13a, | | | | | | |
| | 14, 15a, 16a, 18a, | | | | | | |
| | 34, 35a | | | | | | |

TABLE 102
Preferred Design Solution Combinations Which Can Be Known to Completely Normalize All or
Some Microarray Assay SGDS, DGDS, and DGSS Particular Gene mRNA Transcript
Comparison RASR Values for All or Some Pertinent UNFs and CNFs. Comparison of Oligo
dT Primed LPNs Produced from T-RNA or Isolated mRNA

| | | NFs Which Can Be Ignored | | | Pertinent NFs to Be Determined | | |
|---|---|---|---|---|---|---|---|
| Combination of | | For Normalization | | | and Normalized For | | |
| Assay Design | | SGDS | DGDS | DGSS | SGDS | DGDS | DGSS |
| Solutions | | Comparison | Comparison | Comparison | Comparison | Comparison | Comparison |
| | Comparison of | MLDR | MLDR | SCR | SCR | SCR | PAFR |
| | Type 1 Directly | PL-HKR | PL-HKR | MLDR | PAFR | PAFR | PSAR |
| | Labeled LPNs | PS-HKR | PSSR | PL-HKR | PSAR | PSAR | PS-HKR |
| (1) | Combine | PSSR | LSSR | PSSR | Spatial | PS-HKR | Spatial |
| | Table 53 Design | LLSR | C-HKR | LLSR | Print Tip | Spatial | Print Tip |
| | Solutions | C-HKR | | C-HKR | Print Plate | Print Tip | Print Plate |
| | (a) 2, 4a or b, | | | | Intensity | Print Plate | Intensity |
| | 5a, 6, 8a, c, 9a | | | | Scale | Intensity | Scale |
| | or b, 13b, 14, | | | | | Scale | |
| | 16a, 18a, 34 | | | | | | |
| | Comparison of | MLDR | MLDR | SCR | SCR | SCR | PAFR |
| | Type 2 Directly | PL-HKR | PL-HKR | MLDR | PAFR | PAFR | PS-HKR |
| | Labeled LPNs | PS-HKR | PSSR | PL-HKR | LLSR | LLSR | Spatial |
| (2) | Combine | PSSR | PSAR | PSSR | Spatial | PS-HKR | Print Tip |
| | Table 53 Design | PSAR | C-HKR | PSAR | Print Tip | Spatial | Print Plate |
| | Solutions | C-HKR | | C-HKR | Print Plate | Print Tip | Intensity |
| | (a) 2, 4a or b, | | | LLSR | Intensity | Print Plate | Scale |
| | 5a, 6, 8a, c, 9a | | | | Scale | Intensity | |
| | or b, 13b, 14, | | | | | Scale | |
| | 15a, 16a, 18a, | | | | | | |
| | 34 | | | | | | |
| | Comparison of | MLDR | MLDR | SCR | SCR | SCR | PFAR |
| | Type 1 Indirect | PL-HKR | PL-HKR | C-HKR | C-HKR | C-HKR | SSAR |
| | Label LPNs | PS-HKR | SBNR | MLDR | PAFR | PAFR | PS-HKR |
| (3) | Combine | SBNR | LLSR | PL-HKR | SSAR | SSAR | Spatial |
| | Table 74 Design | LLSR | | SBNR | Spatial | PS-HKR | Print Tip |
| | Solutions | | | LLSR | Print Tip | Spatial | Print Plate |
| | (a) 2, 4a or b, | | | | Print Plate | Print Tip | Intensity |
| | 5a, 6, 8a, d, 9a | | | | Intensity | Print Plate | Scale |
| | or b, 13a, 14, | | | | Scale | Intensity | |
| | 15a, 16a, 18a, | | | | | Scale | |
| | 34, 35a | | | | | | |
| | Comparison of | MLDR | MLDR | SCR | SCR | SCR | PFAR |
| | Type 2 Indirect | PL-HKR | PL-HKR | C-HKR | C-HKR | C-HKR | PS-HKR |
| | Label LPNs | PS-HKR | SBNR | MLDR | PAFR | PAFR | Spatial |
| (4) | Combine | SBNR | SSAR | PL-HKR | LLSR | PS-HKR | Print Tip |
| | Table 74 Design | SSAR | | SBNR | Spatial | LLSR | Print Plate |
| | Solutions | | | SSAR | Print Tip | Spatial | Intensity |
| | (a) 2, 4a or b, | | | LLSR | Print Plate | Print Tip | |
| | 5a, 6, 8a, d, 9a | | | | Intensity | Print Plate | |
| | or b, 13a, 14, | | | | | Intensity | |
| | 15a, 16a, 18a, | | | | | Scale | |
| | 34, 35a | | | | | | |

Note that each described assay design solution combination is often applicable to one assay associated with only SGDS RNA transcript comparisons, or one assay associated with only DGDS RNA transcript comparisons, or one assay associated with only DGSS RNA transcript comparisons, or one assay which is associated with SGDS and DGDS and DGSS RNA transcript comparisons. The microarray assay design solution combinations described in Tables 100-102 represent only a very small fraction of the different microarray assay design solution combinations which can be known to provide improved normalization of microarray SGDS or DGDS or DGSS RNA transcript comparison assay results. As discussed, many more such assay design solution combinations which provide improved normalization of microarray SGDS and/or DGDS and/or DGSS mRNA transcript comparison, or RNA transcript of any kind comparison, assay results, are presented in Tables 54 through 90.

The assay design solution combination associated with a microarray assay determines the following. (i) the validity of the normalization of SGDS and/or DGDS and/or DGSS RNA transcript comparison assay results for the pertinent CNFs. (ii) the completeness of normalization of SGDS and/or DGDS and/or DGSS RNA transcript assay results for pertinent UNFs and CNFs. (iii) the fraction of SGDS and/or DGDS and/or DGSS particular gene RNA transcript comparisons in the assay which can be normalized for all pertinent UNFs and CNFs. (iv) the ease of determining the pertinent CNF and UNF assay values for the SGDS and/or DGDS and/or DGSS particular gene RNA transcript comparisons in the assay. (v) The ease and simplicity of the overall normalization process for the SGDS and/or DGDS and/or DGSS RNA transcript comparisons in the assay. (vi) the biological accuracy of the normalized SGDS and/or DGDS and/or DGSS particular gene RNA transcript comparison assay results obtained from the assay. (vii) the interpretability of the assay measured and normalized SGDS and/or DGDS and/or DGSS particular gene RNA transcript comparison assay results. (viii) the between and within assay intercomparability of assay measured and normalized SGDS and/or DGDS and/or DGSS RNA transcript comparison assay results. (ix) the intercomparability of assay measured and normalized SGDS and/or DGDS and/or DGSS RNA transcript comparison assay results which are obtained with different microarray methods or formats, such as oligonucleotide arrays and cDNA arrays, for which the assay design solution combinations are known.

Here, if a microarray measured and normalized SGDS and/or DGDS and/or DGSS particular gene RNA transcript comparison assay result is biologically accurate, then the following must be true for such an assay result. (a) the normalization is valid and complete. (b) the particular gene RNA transcript comparison N-DGER value can be validly and accurately interpreted as to, the quantitative difference in gene expression extent which exists in the compared cell sample or cell samples for the compared particular gene RNA transcripts, and the direction of regulation change which exists for the comparison. (c) the particular gene RNA transcript comparison N-DGER value can be validly intercompared with other biologically accurate particular gene RNA transcript comparison N-DGER values which have been obtained elsewhere using the same or different microarray or non-microarray methods.

Preferred assay design solution combinations for non-microarray dot blot, northern blot, nuclease protection, and RT-PCR SGDS particular gene mRNA transcript comparison assays are described in Table 91 through 95 and Tables 97 through 100. The design solution used for these described assay design solution combinations are presented in Table 92. These non-microarray assay design solution combinations are also preferred for, DGDS or DGSS particular gene mRNA transcript comparison, and SGDS and/or DGDS and/or DGSS particular gene RNA of any kind comparison, or particular gene RNA transcript of all kinds comparison, non-microarray assays. Note that for non-microarray DGSS particular gene RNA comparison assays, the assay SCR value cannot always be assumed to equal one.

Preferred assay design solution combinations for clone counting method SGDS particular gene mRNA transcript comparison assays are described in Table 99. The design solutions used for these described assay design solution combinations are presented in Table 98. These Table 99 clone counting method assay design solution combinations are also preferred assay design solution combinations for clone counting method DGDS or DGSS particular gene mRNA transcript comparison, or SGDS and/or DGDS and/or DGSS mRNA transcript comparison, clone counting method assays.

Invention Improved Gene Expression Analysis Results and Gene Expression Analysis Comparison Results “Improvement Ripple Effect”:

Further Practices of the Invention. Herein, for simplicity invention improved gene expression analysis and gene expression analysis comparison results are termed invention improved results or improved results. The production of invention improved results causes an “Improvement Ripple Effect”, which extends downstream from the immediate direct production of these improved results, and is due to the use of the improved results. As illustratively described herein, such improved results can provide improvements in quantitation, accuracy, interpretability, reproducibility, intercomparability, utility, and/or biological correctness.

Here, the direct production of the improved results by the methods and means of the invention is termed a zero order application of the methods and means of the invention. This use of the methods and means of the invention in a zero order application, produces zero order application results which are, relative to prior art produced zero order application results, significantly improved, and is a practice of the invention. Examples of such zero order applications of the methods and means of the invention are described extensively herein. One of skill in the art will recognize that these described zero order application examples are only a few of a very large number of possible zero order applications.

A downstream improvement ripple effect is the direct use of improved zero order application results in an application which directly uses zero order application results. Such an application is herein termed a first order application. The use of invention improved zero order application results in a first order application produces first order application results which are, relative to prior art first order application results, significantly improved, and is a practice of the invention. Examples of such first order application use of improved zero order application results include, but are not limited to, the following (a) Producing improved data analysis and data mining analysis method results of all kinds. (b) Producing gene expression profile measurement and identification methods and results of all kinds including disease related gene expression profile measurement methods and results of all kinds. (c) Producing improved bioactive and pharmaceutical candidate or biomarker identification and discovery methods and results of all kinds. (d) Producing improved systems biology analysis methods of all kinds results. (e) Producing improved toxic compound identification and discovery methods and results of all kinds. (f) Producing improved methods and results for developing gene expression based diagnostic test methods of many kinds, including disease detection and characterization methods. (g) Producing improved quality assurance and quality control methods and results for all gene expression analysis applications and toxic, drug, and bioactive molecule discovery and identification methods. These first order applications represent only a few of a great many possible first order applications of improved zero order application methods and results.

A further downstream improvement ripple effect is the use of invention improved first order application results in a still further application which uses one or more improved first order application results. Such an application is herein termed a second order application. The use of invention improved first order application results in a second order application produces second order application results which are, relative to prior art second order application results, significantly improved, and is a practice of the invention. Examples of such second order applications include, but are not limited to, the following. (a) Producing improved systems biology analysis results by using improved data mining analysis results. (b) Producing improved gene regulatory discovery pathway results by using improved data mining analysis and/or systems biology results. (c) Producing improved pharmaceutical or bioactive candidate evaluation and biomarker results by using improved data mining analysis and/or systems biology analysis and/or toxicology analysis and/or safety analysis results. (d) Producing improved pharmaceutical candidate development and biomarker discovery results by using invention improved results from diagnostic tests, data mining analysis, toxicology analysis, systems biology analysis, gene regulatory pathway analysis, QA/QC procedures, and others (e) Producing improved disease related gene expression profile based diagnostic methods by using invention improved results and data mining analysis, systems biology analysis, diagnostic test analysis, biomarker discovery, gene regulatory pathway analysis, QA/QC procedures, and others. (f) Producing improved toxicology and/or safety evaluation results for bioactive compounds by using invention improved results from data mining analysis, systems biology analysis, diagnostic test analysis, biomarker discovery, gene regulatory pathway analysis, QA/QC procedures, and others. These second order applications represent only a few of a great many possible second order applications of invention improved first order application methods and results.

Higher order applications also occur. These higher order applications utilize one or more invention improved lower order application results to produce higher order application results which are, relative to prior art produced higher order application results, significantly improve. This is a practice of the invention. Examples of such higher order applications includes, but are not limited to, the following. Producing improved pharmaceutical, bioactive molecule, or other product higher order application development and/or optimization and/or pharmacologic and/or pharmacokinetic, and/or toxicity study and/or safety study and/or manufacturing and/or QA/QC and/or clinical candidate screening and selection and/or market segment identification and/or drug prescription and use and/or drug efficacy in the patient and/or other results. A more specific example is the production of improved drug manufacturing results by using invention improved lower order application toxicity and/or safety and/or QA/QC and/or diagnostic test and/or pharmacologic and pharmacokinetic and/or biomarker discovery results. Another example is the production of improved prescribed drug efficacy in the patient by using invention improved lower order application drug development and optimization and/or pharmacologic and pharmacokinetic and/or toxicity and/or safety and/or QA/QC and/or manufacturing and/or clinical candidate screening and selection and/or market segment identification and/or drug prescription and/or biomarker discovery results. These higher order applications represent only a few of a great many possible higher order applications of invention improved lower order application results.

Higher order applications are also described in Kohne, U.S. Provisional Appl. 60/689,985, Kohne, U.S. patent application Ser. No. 11/38,203 and Kohne, U.S. patent application Ser. No. 11/383,198, which are hereby incorporated herein by reference in their entireties. The descriptions therein are also applicable to the present invention.

Computer Implementation of Methods for Determining and Using Improved Assay Normalization Techniques

The portions of the invention involving the measurement, determination, and calculation of assay values, normalization factor values, and normalized results for particular assays can be performed using software program or non-software program methods for calculating or determining the respective values. Advantageously, particularly for applications involving large amounts of data, such calculations are carried out using computers loaded with software for performing the various calculations and/or for displaying results. Persons skilled in the field are familiar with performing the relevant calculations, comparing and correlating and interpreting the resulting values, coding the functions in a suitable programming language, and configuring computers to implement the resulting programs and/or to display the relevant results in desired formats. Thus, the calculational steps will not be repeated here. A large number of programs have been developed for performing similar functions based on the types of assay and nucleic acid molecules. If desired, such software can be modified or extended to perform the present calculations.

Thus, the present invention also concerns such computer software, associated databases and data sets, and the use of computers running such software to implement at least portions of the present invention. Such software may be in hard copy (e.g., printing code and/or data) or may be embedded in one or more forms of computer accessible data storage such as random access memory (RAM), read only memory (ROM), magnetic storage media such as computer hard drives, tapes, and floppy disks, optical storage media such as CDs and DVDs and the like, and flash memory devices. The software may be in one or more portions (e.g., modules), which may be in the same physical storage device or in a plurality of different physical storage devices. Likewise, when loaded on a computer, the software may be accessible from a single computer, from any of multiple computers on a LAN or other local network or file transfer connection, or from any of multiple computers over the internet or a WAN or other large scale network. Therefore, the invention also concerns data storage devices and computer systems in which such software is loaded or stored, as well as methods using such software and computer systems to perform the designed functions of the software.

The various functions involved in the present determinations (as well as related determinations) can be performed by separate software programs or other methods, or can be embodied in a single software program or other method. As indicated, one useful software function (or program) is the calculation of improved UNF and/or improved CNF values for particular assays, and their use in normalization of assay results. Such calculations can involve what is essentially a look-up table to find corresponding appropriate experimentally determined values.

In many cases, utilization of the software will involves direct or indirect specification of assay conditions and requirements. The particular parameters which should or must be specified will depend on the particular application and assay times.

Databases & Data Sets

Advantageously, one or more databases (or data sets) can be used which contain data on items used in the respective calculations. Several different types of data which can be advantageously included in such databases are pointed out below. However, a database or set of linked databases need not include all the indicated data in order to be useful, and may include additional data not mentioned. Further, in some implementations, experimental data may be used to derive or otherwise obtain an algorithm at least approximately describing one or more effects (e.g., effects listed below), such that use of the algorithm (e.g., manually or as part of a computer program) may replace use of a corresponding database for at least some range of assay variables. Likewise, when linked with a computer program, a program may be configured to interpolate between data points (e.g., using any of a variety of known and available interpolation algorithms) to approximate effects for conditions which are not exactly or not completely represented in the database.

Sequence and Sequence-Related Data: One such database (or set of databases) or data set (of set of data sets) contains sequence and/or sequence related data for the RNA of interest, e.g., for a particular cell type of interest. Such a database can, for example, include sequence information for RNA (e.g., mRNA and/or regulatory RNA) from a particular gene, from a set of a plurality of genes, or from all or essentially all expressible gene in cell. (For purposes of this discussion, unless clearly indicated to the contrary, reference to a database shall include one or more databases (e.g., one or more databases accessible from a computer or computer system), and shall also include the data sets stored in the database. Further, at least some of the information may be in publicly accessible databases, such as in GenBank and related or similar databases.

Likewise, such database may contain such sequence information for a plurality of different types of cells. For example, such cells may be from various source organisms (e.g., human, mouse, rat, pig, ape, monkey, or other non-human mammal, bacteria, yeast), may be from different tissues in an organism or organisms), may be from a cell line (e.g., an immortal or immortalized cell line) may be normal, may contain gene variants (e.g., allelic variants, splice variants, mutations, and the like), may be pathological or diseased (e.g., cancer or other neoplastic cell), may be infected with one or more microorganisms (e.g., viral, bacterial, or other microorganism), may have been treated with one or more chemicals and/or particular physical conditions, may be from an organism which has been treated with or subjected to one or more particular chemical, drug, and/or environmental conditions, and/or may be prokaryotic or eukaryotic, among others.

Such database can contain information on variants and processed forms of particular genes and RNA produced from those genes, e.g., allelic variants, mutants with detectable phenotypic effect, splice and other RNA processing variants, homologous forms, and the like.

Such database can include data describing the nucleotide sequence, length, and/or nucleotide composition of nucleotide probes, e.g., capture probes. Thus, for example, the database can include such data for the capture nucleic acid probes in capture spots of interest in a microarray (preferably for each capture spot of interest).

Nucleic acid length, sequence, composition, and structure effects on hybridization: Likewise, a database may contain data describing the effect of some or all of the length, sequence, composition, and secondary structure of the nucleic acid molecule(s) on the kinetics and/or completeness of hybridization of cell sample or reference or standard particular gene target (PG-T) molecules with a complementary oligomer, e.g., a cDNA capture probe which is immobilized on a microarray (MA), under assay conditions and/or conditions which can be correlated with assay conditions. Such effect data may be for unlabeled and/or labeled (directly or indirectly) target or complementary nucleic acid molecules.

Label effects: Such databases may also include data (e.g., a data set(s)) describing the effect of the label density and/or label location and/or label type of a PG-T on the kinetics and/or completeness of hybridization of the target with a complementary oligonucleotide, e.g., a capture probe immobilized on a microarray. Data for such labeling can be directed to any type of label, including direct labels and indirect labels.

A database may also include data describing the effect of label density on the magnitude of the signal intensity associated with the target under assay conditions. Such label density effects on signal intensity may be present for a variety of different labels, e.g., fluorescent, luminescent, phosphorescent, as well as others. As indicated in connection with hybridization effects of label density, the labels may be either direct or indirect labels.

Data included in a data base can describe the relationships between the sample target labeling conditions and compositions, and the efficiency of label molecule incorporation in different PG-T molecules, e.g., molecules which have different nucleotide sequences, composition, and/or secondary structures. Such data applies to many different types of labels in which a direct or indirect label component is incorporated, including, for example, fluorescent, phosphorescent, and radioactive labels.

Another useful data set describes the relationship between the quantity of PG-T molecules measured under assay conditions and the intensity of signal obtained. Thus, such data can describe the linearity or non-linearity of signal intensity as a function of labeled molecule amount or concentration.

Nucleic Acid Degradation Parameters: Data related to nucleic acid degradation (e.g., RNA degradation) can also be useful. For example, in relation to undegraded sample RNA and degraded sample RNA, data describing or characterizing the relationship between the average nucleotide length of a samples total or total target RNA molecules, and the average nucleotide length of particular gene RNA which are present in respective sample pools can be usefully included. Advantageously, the data set can include such data covering a range of degrees of degradation. Similar data sets for cell sample and/or standard cDNA or cRNA preparations are also useful.

Measured and Derived Assay Parameters

Valid normalization involves a number of different measured assay parameters which can be utilized in normalization methods, as well as parameters derived from such measurements. The particular assay parameters applicable to a particular assay will be recognized by one of skill in the art in view of the description herein. For example, different microarray assay systems will involve different combinations of measured parameters, generally for each sample of interest.

Such assay parameters can include, for example:
(i) the average length of the nucleic acid molecules in the sample nucleic acid (e.g., total RNA or total target RNA) or the average length of one or more specific reference PG-T molecules present in the prep (e.g., PG-T prep).
(ii) the fraction of mRNA for each particular gene in a sample (or each sample) which is significantly polyadenylated (polyA or PA).
(iii) the total RNA/intact cell.
(iv) the total mRNA/intact cell.
(v) the total DNA/intact cell.
(vi) the sample RNA and/or mRNA isolation efficiency (REI).
(vii) the sample DNA isolation efficiency.
(viii) the synthesis yield fraction for cDNA or cRNA.
(ix) the amount of sample RNA, cDNA, or cRNA analyzed in the assay hybridization solution.
(x) the maximum signal activity which is associated with a target labeled signal generation molecule when measured under assay conditions.
(xi) the average label density (ALD) associated with a sample labeled target preparation.
(xii) the relationship between the assay measured signal activity and the input RNA concentration for a PG-T.
   Program Functions

The software program can be readily configured as desired to provide appropriate functions for the intended application.

In particular application it will be desired to calculate assay values for one or more UNFs, such as SCR, STMR, PAFR, MLDR, PL-HKR, PS-HKR, PSAR, PSSR, LLNR, LLSR, SPNR, and SSAR and/or CNFs.

In order to determine such values, it is desirable to have and implement algorithms which perform the following functions, e.g., using methods are described herein:
(i) determine the average nucleotide length for a PG-T molecule population in a sample target preparation.
(ii) determine the average NS, NC, and SS for a PG-T molecule population in a sample target preparation.
(iii) determine the label density (LD) for a PG-T molecule population in a sample target preparation.
(iv) determine the average mass of a PG-T nucleic acid which can hybridize to one spot immobilized complementary capture probe molecule.
(v) determine, for a sample target preparation, the effect of the NL, NS, NC, SS, and/or LD on the kinetics and completeness of hybridization of PG-T molecules to spot immobilized complementary capture probes.
(vi) determine th, for a PG-T in a sample target preparation, the effect of the PG-T LD value on the signal intensity produced by the PG-T.
(vii) determine the number of cell equivalents (CE) of sample target RNA, cDNA, or cRNA which are analyzed in the assay hybridization solution.
(viii) determine the proportionality of the relationship between the assay input RNA, dDNA, or cRNA concentration and the assay measured signal activity for spot hybridized PG-T molecules.

Exemplary Data and Algorithms Implemented in Software for Determining and Normalizing for the UNF SCR Value for a Microarray Assay

An exemplary application concerns normalizing the UNF SCR for a microarray assay of interest. This description is illustrative of determining one of the more complex UNFs.

Depending on the particularities of the microarray assay system and design, different combinations of the following data is used to determine the SCR.
(i) Each samples total RNA/intact cell content.
(ii) Each samples total mRNA/intact cell content.
(iii) Each samples total DNA/intact cell content.
(iv) The total DNA in each sample of interest.
(v) The RNA isolation efficiency for each sample of interest.
(vi) The DNA isolation efficiency for each sample of interest.
(vii) The cDNA or cRNA synthesis yield fraction (YF) for each sample target cDNA or cRNA target preparation.
(viii) the amount, for each sample, of RNA, cDNA, or cRNA analyzed in the assay hybridization solution.

The relevant data is then processed using the respective algorithms for determining the average nucleotide length of each sample's target preparation molecules (e.g., as described herein), determining the number of sample cell equivalents which are present in the assay hybridization solution for each sample, determining the assay SCR value for a cell sample comparison, and normalizing the microarray assay results for the SCR.

Exemplary Data and Algorithms Implemented in Software for Improved Determining and Normalizing for the Pertinent CNF Values for a Microarray Assay

A related exemplary application concerns determining pertinent improved CNF values for an assay, and use of those values in normalizing assay results for such CNF value(s).

As described for UNFs above, different combinations of data will be applicable for different microarray systems. Such data can include different combinations of the following:
(i) Total mRNA/intact cell for each sample.
(ii) Microarray assay results for properly positioned replicate standard and PG assays.
(iii) Microarray assay signal activity results for replicated properly positioned standard assay results which represent greatly different RNA inputs.
(iv) The overall microarray assay results for all sample expressed PGs which have been normalized for assay pertinent UNFs, and separate compilations of a) the total signal intensity associated with all of the upregulated PGs in the assay, and
(iv) the total signal intensity associated with all of the downregulated genes in the assay.
(v) A data set specifying the particular print tip which was used to produce each PG and standard capture probe spot and the spatial location of the capture probe spot on the microarray surface.
(vi) A data set for cDNA microarrays specifying the microplate well and the print tip and the spatial location on the microarray for each PG and standard capture probe spot in the array.

Such data is used to assess the validity and/or assay value for particular pertinent CNFs. Thus, such CNFs can be improved, such as by establishing that the CNF is valid, showing that a normalization process utilizing the CNF is valid, reducing the likelihood that the CNF or associated normalization process is invalid, and/or providing improved CNF assay values.

The improvements engendered by the improved UNFs and CNFs allows improvement in assay results, and thus may provide improved interpretability, reliability, and the like. The improvements in assay results can be provided by the improvements in normalization of the results by the methods as described herein. Such improved results are typically due to more complete and/or more valid or more likely to be valid normalization.

Kits for Performing Assays with Improved Normalization, Validation, Calibration, and/or Corroboration

Practice of the methods described above for improved normalizing of a variety of different assays can involve changes or additions in the materials used for performing the assays or in performing associated determinations relating to improved normalization, validation, calibration, and/or corroboration of assay results. Components and/or instructions for carrying out processes can be useful incorporated and supplied in kit form, e.g., an assay kit with additional components and/or instructions for performing the further functions. Alternatively, separate assay kits can be provided for performing the improved normalization, validations, or corroboration of separate assay results. In most cases, the kits will be packaged or otherwise assembled together. A kit may be single use, but in many case will have sufficient components for carrying out multiple assays, e.g., at least 2, 3, 5, 10, 20, 50, or 100 such assays.

Thus, in many cases, the kit will include one or more components for carrying out the assay, along with instructions and/or materials for carrying out improved normalization and/or for determining that a normalization process is improved or valid and/or for calibrating the assay and/or for corroborating results for basic assay and/or for evaluating the performance characteristics of an assay. Such instructions may be in various forms, e.g., written and/or graphic and/or electronic, and one or more forms may be used for a particular assay kit. Electronic forms may be provided directly, or may be provided in the form of directions for accessing the instructions (e.g., internet site access directions). Either as part of the instructions or separately, computer software for carrying out improved normalization and/or the other functions indicated herein can be supplied.

The invention also concerns the instructions separately. For example, such instructions may be provided on a web site or in the form of a printed or electronic manual, e.g., a book or booklet, which may contain instructions for additional assays, information on assay systems, evaluation reports, and/or other information, or as included information in a catalog or similar format.

Those familiar with such assays are familiar with components which are commonly included in commercial assay kits, such as microarrays, enzymes such as a reverse transcriptase, a DNA polymerase (e.g., a heat stable polymerase), a nuclease, and the like, a prepared affinity medium (e.g., a nucleic acid purification column), one or more buffers (in dry or liquid form), and the like, so the basic assay reagents will not be further described here. In certain cases, the assay kit will include the components for improvement in conjunction with components from an existing commercial assay kit, e.g., a kit provided by recognized assay kit providers (or their successor entities).

As indicated, the kit can include physical components used in the assay and/or components for determining improved normalization factors related to the assay. Those components will depend, in part, on the type of assay for which the kit is intended, e.g., microarray, RT-PCR, nuclease protection, clone counting, affinity media separation (e.g., hydroxyapatite), or ELISA or similar assay, and the like.

A number of different components for performing improved normalization and/or other assay improvements are indicated in the Summary and in the claims herein. Some general categories of components which can be advantageously incorporated in an assay kit include, without limitation, improved and/or characterized nucleic acid standards; characteristic data concerning such nucleic acid standards; reagents for preparing improved nucleic acid molecules such as oligonucleotides; cell sample, enriched, purified, or standard nucleic acid preparations; characteristic data concerning such cell sample, enriched, purified, or standard nucleic acid preparations; and/or reagents for determining characteristic data for such cell sample, enriched, purified, or standard nucleic acid preparations.

In addition, combination or separate assay kits can include components (e.g., reagents and/or instructions) for performing a corroboration or validation assay or test. In such cases, instructions for performing valid corroboration assays or tests can advantageously be included or otherwise made available. For example, a corroboration assay for a microarray can be an RT-PCR assay (or the converse). Similarly, a corroboration assay for either a microarray or RT-PCR assay may be an affinity separation assay (e.g. hydroxyapatite), a centrifugation separation assay, nuclease protection assay, an ELISA assay, or the like.

Thus, a large number of useful assay kits can be constructed which provide the present assay improvements and/or corroboration. All such assay kits are within the present invention.

### CONCLUSION

While the present invention has been described in terms of a large number of different particular embodiments, it is not intended that the invention be limited to these embodiments. These multiple embodiment descriptions are but a small fraction of the present invention embodiments which are possible, and numerous modifications of the described embodiments which are within the scope of the invention will be apparent to those skilled in the art. As discussed, a very large number of different implementations of the present invention are possible depending on, the type of gene expression analysis procedure used, the types of cell samples compared, the biological quality of the compared cell samples, the T-RNA or mRNA content per cell for the analyzed call samples, the RNA isolation process, the RNA quality, the RNA type analyzed, the amount of RNA analyzed, the type of RNA equivalent compared, the primer used to produce the RNA equivalent, the process used to produce the RNA equivalents, the type of label used, the number of different label types used, the type of standard used, the number of standards used, how the standards are used, and other factors.

For the purpose of explanation, the foregoing explanation used specific nomenclature to provide a thorough understanding of the invention and its many embodiments. However, it will be apparent to one of skill in the art that this nomenclature and specific details are but one way to describe and implement the invention. Thus, the foregoing descriptions of particular embodiments of the present invention are presented for the purpose of illustration and description, and they are not intended to be exhaustive or to limit the invention to the precise forms disclosed, and obviously many modifications and variations are possible as a result of the above teachings. The embodiments presented were selected and described in order to best explain the principles of the present invention and its practical applications, and to thereby enable others skilled in the art to best practice the invention and various embodiments with various modifications, as are suited to the particular use contemplated.

For simplicity the abbreviations PG (particular gene), S (standard), NF (normalization factor), CNF (prior art considered normalization factor), UNF (prior art unconsidered normalization factor), RN (the number of PG RNA transcript molecules present in a cell sample RNA transcript preparation aliquot). SGDS (same gene different cell sample), SGDS (same gene different cell sample), and DGSS (different gene same cell sample), will be utilized in the claims. In addition, gene expression analysis results refer to mRNA Transcript Number (mTN) and/or RN and/or mRNA abundance and/or NAS values for one or more particular genes in a cell sample. Further, gene expression comparison analysis results refer to mTN and/or mRNA abundance and/or NAS and/or NASR and/or N-DGER values for one or more particular genes in compared cell samples.

### IV. REFERENCES

1) Lewin, Gene Expression vol. 2, Wiley-Interscience New York (1980)
2) Lavorgna et al, In Search of Antisense. Trends in Bioch. Sci. 29:88-94 (2004)
3) Lockhart et al, Genomics, Gene Expression and DNA Arrays. Nature Insight. 405:827-836 (2000)
4) Duggan et al, Expression Profiling Using cDNA Microarrays. Nature Genetics Supplement. 21: 10-14 (1999)
5) Granjeaud et al, Expression Profiling: DNA Arrays in Many Guises. Bioessays. 21: 781-790 (1999)
6) Lockhart et al, Expression Monitoring by Hybridization to High-Density Oligonucleotide Arrays. Nature Biotech. 14: 1675-1680 (1996)
7) Botwell et al, DNA Micro Arrays. Cold Spring Harbor Press, New York (2003)
8) Lorkowski et al, Analysis Gene Expression, A Handbook of Methods Possibilities and Pitfalls vol 1 and 2. Wiley-VCH (2003)
9) Hastie et al, The Expression of Three Abundance Classes of Messenger RNA in Mouse Tissues. Cell. 9: 761-774 (1976)
10) Neidhart et al, Physiology of the Bacterial Cell, A Molecular Approach. Sinauer Assoc. Inc Pub, Sunderland, Mass. (1990)
11) Mandelstam et al, Biochemistry of Bacterial Growth Third Edition. Blackwell Scientific Publications, London (1982)
12) Eisen et al, DNA Arrays for Analysis of Gene Expression. Methods in Enzym. 303: 179-205 (1999)
13) Sambrook et al, Molecular Cloning, A Laboratory Manual Third Edition, vol 1, 2, 3. Cold Spring Harbor Press, New York (2001)
14) Johnson et al, Changes in RNA in Relation to Growth of the Fibroblast. I. Amounts of mRNA, rRNA, and tRNA in Resting and Growing Cells. Cell 1: 95-100 (1974)
15) Leslie, The Nucleic Acid Content of Tissues and Cells. In, The Nucleic Acids, Chemistry and Biology, vol II Chap. 16:1-50. Eds. Chargaff and Davidson (1955)
16) Todd et al, Challenges of Single-Cell Diagnostics: Analysis of Gene Expression. Trends in Mol. Med. 8: 254-257 (2002)
17) Reue, mRNA Quantitation Techniques: Considerations for Experimental Design and Application. J. Nutrition, 128: 2038-2044 (1998)
18) Darling et al, Nucleic Acid Blotting, The Basics. IRL Press Oxford (1994)
19) Wodicka et al, Genome-Wide Expression Monitoring in Saccharomyces Cerevisiae. Nature Biotech. 15: 1359-1367 (1997)
20) Kwloch et al, Quantitative Analysis of Specific mRNAs By Ribonuclease Protection. Meth. in Enzym. 225:294-303 (1993)
21) Durnam et al, A Practical Approach For Quantitative Specific mRNAs By Solution Hybridization. Anal. Biochm. 131:385-393 (1983)
22) Taniguchi et al, Quantitative Assessment of DNA Microarrays-Comparison With Northern Blot Analysis. Genomics, 71: 34-39 (2001)
23) Donson et al, Comprehensive Gene Expression Analysis By Transcript Profiling. Plant Mol. Biol. 48: 75-97 (2002)
24) Brenner et al, Gene Expression Analysis By Massively Parallel Signature Sequencing (MPSS) on Microbead Arrays. Nature Biotech. 18: 630-634 (2000)
25) Yamanoto et al, Use of Serial Analysis of Gene Expression (SAGE) Technology. J. Immun. Methods. 250: 45-66 (2001).
26) Zhang et al, Gene Expression Profiles In Normal And Cancer Cells. Science 276: 1268-1272 (1997)
27) Velculescu et al, Analysis of Human Transcriptomes Nature Gen. 23: 387-388 (1999)
28) Basset et al, Gene Expression Informatics—It's All In Your Mine Nature Gen. Suppl. 21: 51-55 (1999)
29) Brazma et al, Minimum Information About A Microarray Experiment (MIAME)—Towards Standards for Micro Array Data. Nature Gen. 29: 365-371 (2001).
30) Schena, Microarray Biochip Technology. Eaton Publishing, Natick Mass. (2000).
31) Quackenbush, Microarray Data Normalization and Transformation. Nature Gen. Suppl. 32: 496-501 (2002)
32) Knudsen, Guide to Analysis of DNA Microarray Data, Second Edition. Wiley-Liss (2004)
33) Causton et al, A Beginners Guide. Microarray Gene Expression Data Analysis. Blackwell Pub. (2003)
34) Speed, Statistical Analysis of Gene Expression Microarray Data. Chapman and Hall/CRC (2003)
35) Draghici, Data Analysis Tools For DNA Microarrays. Chapman and Hall/CRC (2003)
36) Kohane et al, Microarrays For An Integrated Genomics. Bradford Book, MIT Press (2003)
37) McLachlan et al, Analysing Microarray Gene Expression Data. Wiley Interscience (2004)
38) Burczynski, An Introduction To Toxicogenomics. CRC Press (2003)
39) Eickoff et al, Normalization of Array Hybridization Experiments In Differential Gene Expression Analysis. Nuc. Acid Res. 27: e33 (1999)
40) Brazma et al, Minireview: Gene Expression Data Analysis. FEBS Letters. 480: 17-24 (2000)
41) Schuchhardt et al, Normalization Strategies For cDNA Microarrays. Nuc. Acid Res. 28: e47 (2000)
42) Beissbarth et al, Processing and Quality Control of DNA Array Hybridization Data. Bioinformatics 16: 1014-1020 (2000)
43) Hill et al, Evaluation of Normalization Procedures For Oligonucleotide Array Data Based On Spiked cRNA Controls. Genome Biology. 2 (12): 0055.1-0055.13 (2001)
44) Yang et all, Normalization For cDNA Microarray Data. In Microarrays: Optical Technologies and Informatics. Proc. Spie. Vol 4266: 141-152 (2001)
45) Fang et al, A Model-Based Analysis of Microarray Experimental Error and Normalization. Nuc. Acid Res. 31: e96 (2003)
46) Zien et al, Centralization: A New Method For The Normalization of Gene Expression Data. Bioinformatics. 17: 5323-5331 (2001)
47) Chudin et al, Assessment Of The Relationship Between Signal Intensities and Transcript Concentration For Affymetrix Genechip Arrays. Genome Biol. 3(1): 0005.1-0005.10 (2001)
48) Tseng et al, Issues in cDNA Microarray Analysis: Quality Filtering, Channel Normalization, Models of Variations and Assessment of Gene Effects. Nuc. Acid. Res. 29: 2549-2557 (2001).
49) Li et al, Model-Based Analysis of Oligonucleotide Arrays: Model Validation, Design Issues and Standard Error Application. Genome Biol. 2(8): 0032.1-0032.11 (2001)
50) Quackenbush, Computational Analysis of Microarray Data. Nature Reviews. Genetics 2: 418-427 (2001)
51) Finkelstein et al, Microarray Data Quality Analysis: Lessons From The AFGC Project. Plant Mol. Biol. 48: 119-131 (2002)
52) Hoffmann et al, Profound Effect of Normalization on Detection of Differentially Expressed Genes In Oligonucleotide Microarray Data Analysis. Genome Biol. 3 (7): 0033.1-0033.11 (2002)
53) Tsodikov et al, Adjustments and Measures of Differential Expression For Microarray Data Bioform. 18: 251-260 (2002)
54) Kroll et al, Ranking: A Closer Look On Globalisation Methods For Normalization of Gene Expression Arrays. Nuc. Acid Res. 30 (11): e50 (2002)
55) Colantouni et al, Local Mean Normalization of Microarray Element Signal Intensities Across An Array Surface: Quality Control and Correction of Spatially Systemic Artifacts. Biotechniques 32: 1316-1323 (2002)
56) Workman et al, A New Non-Linear Normalization Method For Reducing Variability In DNA Microarray Experiments. Genome Biol. 3(9): 0048.1-0048.16 (2002)
57) Wang et al, Iterative Normalization of cDNA Microarray Data. IEEE Trans. On Inform. Tech. In Biomedicine. 6: 29-37 (2002)
58) NAEF et al, Empirical Characterization of the Expression Ratio Noise Structure In High-Density Oligonucleotide Arrays. Genome Biol. 3 (4): 0018.1-008.11 (2002)
59) Kuo et al, Analysis of Matched mRNA Measurements From Two Different Microarray Technologies. Bioinformatics. 18: 405-412 (2002)
60) Zhou et al, Match Only Integral Distribution (MOID) Algorithm For High Density Oligonucleotide Array Analysis. BMC Informatics. 3(3). (Jan. 22, 2002)
61) Yuen et al, Accuracy and Calibration of Commercial Oligonucleotide and Custom cDNA Microarray Microarrays. Nuc. Acid Res. 30 (10): e48 (2002)
62) Yang et al, Normalization for cDNA Microarray Data: A Robust Composite Method Addressing Single and Multiple Slide Systemic Variation. Nuc. Acid Res. 30 (4): e15 (2002)
63) Hubbel et al, Robust Estimators for Expression Analysis. Bioinform. 18: 1585-1592 (2002)
64) Kothapalli et al, Microarray Results: How Accurate Are They? BMC Bioinformatics. 3 (22): (Aug. 23, 2002)
65) Irizarry et al, Exploration, Normalization, and Summaries of High Density Oligonucleotide Array Probe Level Data. Biostatistics. 4: 249-264 (2003)
66) Rajagopalan, A Comparison of Statistical Methods for Analysis of High Density Oligonucleotide Array Data. Bioinformatics. 19: 1469-1476 (2003)
67) Datta, Statistical Techniques for Microarray Data: A Partial Overview. Communications in Statistics, Theory and Methods. 32(1): 263-280 (2003)
68) Geller, Transformation and Normalization of Oligonucleotide Microarray Data. Bioinformatics. 19(14): 1817-1823 (2003)
69) Berger et al, Optimized Lowess Normalization Parameter Selection for DNA Microarray Data. BMC Bioinformatics. 5(194): (Dec. 9, 2004)
70) Irizarry et al, Summaries of Affymetrix Genechip Probe Level Data. Nuc. Acid Res. 31(4):e15(2003)
71) Mei et al, Probe Selection for High Density Oligonucleotide Arrays. Proc. Natl. Acad. USA 100(20): 11237-11242(2003)
72) Ye et al, Applications of DNA Microarrays in Microbial Microsystems. J. Microbiol. Methods. 47:257-272 (2001)
73) Souaze et al, Quantitative RT-PCR: Limits and Accuracy Biotechniques. 21 (2): 280-285 (1996)
74) Freeman et al, Quantitative RT-PCR: Pitfalls and Potential. Biotechniques. 26(1): 112-124 (1999)
75) Vandesompele et al, Accurate Normalization of Real Time Quantitative RT-PCR Data Geometric Averaging of Multiple Internal Control Genes. Genome Biology. 3(7): 0034.1-0034.11 (2002)
76) Crawford et al, Multiplex Standardized RT-PCR for Expression Analysis of Many Genes in Small Samples. Bioch. Biophys. Res. Comm. 293: 509-516 (2002)
77) Gallinella et al, Calibrated Real Time PCR Evaluation of Parvovirus B19 Viral Load. Clinical Chem. 50 (4): 759-962 (2004)
78) Audic et al, Significance of Digital Gene Expression Profiles. Genome Research. 7: 986-995 (1997)
79) Claverie, Computational Methods for the Identification of Differential and Coordinated Gene Expression. Human Molec. Genetics. 8(10): 1821-1832 (1999)
80) Ewing et al, Large-Scale Statistical Analyzes of Rice ESTs Reveal Correlated Patterns of Gene Expression. Genome Research. 9: 950-959 (1999)
81) Stollberg et al, A Quantitative Evaluation of Sage. Genome Research. 10: 1241-1248 (2000)
82) Man et al, Power Sage: Comparing Statistical Tests for Sage Experiments. Bioinformatics. 16(11): 953-959 (2002)
83) Hegde et al, A Concise Guide to cDNA Microarray Analysis. Biotechniques. 29: 548-562 (2002)
84) Hamaden et al, Toxicogenomics, Principles and Applications. Wiley-Liss (2004)
85) Boultwood et al, Molecular Analysis of Cancer. Methods in Molecular Medicine. Vol 68 (2002), Humana Press
86) Appasani et al, Perspectives in Gene Expression. Eaton Press (2003)
87) Warrington et al, Microarrays and Cancer Research. Biotechniques Press (2002)
88) Smyth et al, Normalization of cDNA Microarray Data. Methods. 31: 265-273 (2003)
89) Finkelstein et al, Iterative Linear Regression By Sector: Renormalization of cDNA Microarray Data and Cluster Analysis Weighted By Cross Homology. In Methods of Microarray Data Analysis, Papers From Camda 2000. Eds. Lin et al, Kluwer Academic Press, Pg 57-68 (2002)
90) Ramdas et al, Sources of Nonlinearilty in cDNA Microarray Expression Measurements. Genome Biology. 2 (11): 0047.1-0047.7 (2001)
91) Brown et al, Image Metrics in the Statistical Analysis of DNA Microarray Data. Proc. Nat. Acad. Sci. Us. 98: 8944-8949 (2001)
92) Tran et al, Microarray Optimizations: Increasing Spot Accuracy and Automated Identification of True Microarray Signals. Nuc. Acid Res. 30(12): e54 (2002)
93) Wit et al, Statistical Adjustment of Signal Censoring in Gene Expression Experiments. Bioinformatics. 19(9): 1055-1060 (2003)
94) Schadt et al, Analysing High Density Oligonucleotide Gene Expression Array Data. J. Cellular BCHM. 80: 192-202 (2000)
95) Wang et al, Quantitative Quality Control in Microarray Image Processing and Data Acquisition. Nuc. Acid Res. 29(15): e75 (2001)
96) Ivell, A Question of Faith—Or The Philosophy of RNA Controls. J. Endocrin. 159: 197-200 (1998)
97) Brooks et al, Secondary Structure in the 3'UTR of EFG and the Choice of Reverse Transcriptases Affect the Detection of Message Diversity By RT-PCR. Biotechniques. 19: 806-815 (1995)
98) Curry et al, Low Efficiency of the Moloney Murine Leukemia Virus Reverse Transcriptase During Reverse Transcription of Rare t(8;21) Fusion Gene Transcripts. Biotechniques. 32: 768-774 (2002)
99) Madison et al, Lambda RNA Internal Standards Quantify Sensitivity and Amplification Efficiency of Mammalian Gene Expression Profiling. Biotechniques. 25: 504-514 (1998)
100) Zhao et al, Optimization and Evaluation of T7 Based RNA Linear Amplification Protocols For cDNA Microarray Analysis. BMC 3(31): (Oct. 30, 2002)
101) Xiang et al, A New Strategy to Amplify Degraded RNA From Small Tissue Samples For Microarray Studies. Nuc. Acid Res. 31(9): e53 (2003)
102) Wilson et al, Amplification Protocols Introduce Systematic But Reproducible Errors Into Gene Expression Studies. Biotechniques. 36(3): 498-506 (2004)
103) Pannetier et al, Quantitative Titration of Nucleic Acids By Enzymatic Amplification Reactions Run to Saturation. Nuc. Acid Res. 21(3): 577-583 (1993)
104) Mullis et al, The Polymerase Chain Reaction. Birknäuser, Boston. (1994)
105) Hayward et al, Kinetics of Competitive Reverse Transcriptase-PCR. In PCR Applications, Protocols For Functional Genomics. ED. Innis et al Academic Press, Chapter 15: 231-261 (1999)
106) Mattews et al, Persistent DNA Contamination In Competitive RT-PCR Using cRNA Internal Standards: Identity, Quality, and Control. Biotechniques. 32: 1412-1417 (2002)
107) Bustin, Absolute Quantitation of mRNA Using Real-Time Reverse Transcription PCR Assays. J. Mol. Endocrin. 25: 169-193 (2000)
108) Zhang et al, A Novel Medium Throughput Quantitative Competitive PCR Technology To Simultaneously Measure mRNA Levels From Multiple Genes. Nuc. Acid Res. 30(5): e20 (2002)
109) Bustin, Quantification of mRNA Using Real-Time Reverse Transcription PCR (RT-PCR): Trends and Problems. J. Mol. Endocrin. 29: 23-39 (2002)
110) Nam et al, Oligo (dT) Primer Generates A High Frequency of Truncated cDNAs Through Internal Poly (A) Priming During Reverse Transcription. Proc. Natl. Acad. Sci. US. 99(9): 6152-6156 (2002)
111) Stahlberg et al, Properties of Reverse Transcription Reaction In mRNA Quantification. Clinical Chem. 50(3): 509-515 (2004)
112) Weissensteiner et al, PCR Technology, Current Innovations. CRC Press 2004.
113) Mimmack et al, Quantitative Polymerase Chain Reaction: Validation of Microarray Results From Postmortem Brain Studies. Biol. Psychiatry. 55: 337-345 (2004)
114) Dieffenbach et al, PCR Primer Second Edition. Cold Spring Harbor Press (2003)
115) Ambion Inc. Technical Bulletin #185. Obtained From Ambion Web Site In February 2004.
116) O'Connel, RT-PCR Protocols. Humana Press, Methods In Molecular Biology vol. 193: page 102 (2002)
117) Application Note From Applied Biosystems Inc. Website. “Amplification Efficiency of TaqMan Gene Expression Assays.” (Obtained in Late 2004.)
118) Bubner et al, Twofold Differences Are The Detection Unit For Determining Transgene Copy Numbers In Plants By Real-Time PCR. BMC Biotechnology. 4(14): (Jul. 14, 2004)
119) Johnson et al, Quantitation of Dihydropyrimidine Dehyprogenase Expression By Real-Time Reverse Transcription PCR. Anal. Biochem. 278: 175-184 (2000)
120) Ding et al, Quantitative Analysis of Nucleic Acids—The Last Few Years of Progress. J. BCHM. And Molec. Biology. 37: 1-10 (2004)
121) Nogva et al, Potential Influence of the First PCR Cycles In Real Time Comparative Gene Quantifications. Biotechniques. 37(2): 246-253 (2004)
122) Zhang et al, Two Variants of Quantitative Reverse Transcriptase PCR Used To Show Differential Expression of α-, B- and Y-Fibrindogen Genes In Rat Liver Lobes. Biochem. J. 321: 769-775 (1997)
123) Zhang et al, A Novel Highly Reproducible Quantitative Competitive RT-PCR System. J. Mol. Biol. 274: 338-352 (1997)
124) Livak et al, Analysis of Relative Gene Expression Data Using Real-Time Quantitative PCR and The 2^{ΔΔC}T Method. Methods. 25: 402-408 (2001)
125) ØVSTEBØ et al, PCR-Based Calibration Curves For Studies of Quantitative Gene Expression In Human Monocytes: Development and Evaluation. Clin. Chem. 49(3): 425-432 (2003)
126) Pierson et al, Experimental Validation of Novel and Conventional Approaches To Quantitative Real-Time PCR Data Analysis. Nuc. Acid Res. 31(14): e73 (2003)
127) Kainz, The PCR Plateau Phase-Toward An Understanding of its Limitations. Biochem. Biophys. ACTA. 1494: 23-37 (2000)
128) Liu et al, Heterogeneous Expression of Tandem-Pore K+ Channel Genes in Adult and Embryonic Rat Heart Quantified By Real-Time PCR. Clin. and Exptl. Pharm. And Physiol. 31: 174-178 (2004)
129) Ramakers et al, Assumption-Free Analysis of Quantitative Real-Time PCR Data. Neurosciences Letters. 339:62-66 (2003)
130) Pan et al, How Many Replicates of Arrays Are Required To Detect Gene Expression Changes in Microarray Experiments? A Mixture Model Approach. Genome Biol. 3(5): 0022.1-0022.10 (2002)
131) Spanakis, Problems Related To The Interpretation of Autoradiographic Data on Gene Expression Using Common Constituitive Transcripts as Controls. Nuc. Acid Res. 21(16): 3809-3819 (1993)
132) Speiss et al, Amplified RNA Degradation In T7-Amplification Methods Results In Biased Microarray Hybridizations. BMC Genomics. 4(44): (Nov. 10, 2003)
133) Chuaqui et al, Post-Analysis Followup and Validation of Microarray Experiments. Nature Gen. Suppl. 32: 509-514 (2002)
134) Lee et al, Control Genes and Variability: Absence of Ubiquitous Reference Transcripts In Diverse Mammalian Expression Studies. Genome Research. 12: 292-297 (2001)
135) Moreau et al, Comparison and Meta-Analysis of Microarray Data: From the Bench To The Computer Desk. Trends in Genetics. 19(10): 570-577 (2003)
136) Nadon et al, Statistical Issues With Microarrays: Processing and Analysis. Trends in Genetics. 18: 265-271 (2002)
137) Goryachev et al, Unfolding of Microarray Data. J. Computational Biology. 8(4): 443-461 (2001)
138) Baldi et al, DNA Microarrays and Gene Expression, From Experiments To Data Analysis and Modeling. Cambridge U. Press (2002)
139) Selinger et al, On The Complete Determination of Biological Systems. Trends in Biotech. 21(6): 251-254 (2003)
140) Skrypina et al, Total RNA Suitable For Molecular Biology Analysis. J. Biotech. 105: 1-9 (2003)
141) Schoop et al, Moderate Degradation Does Not Preclude Microarray Analysis of Small Amounts of RNA. Biotechniques. 35: 1192-1201 (2003)
142) Miller et al, Methods To Optimize The Generation of cDNA From Postmortem Human Brain Tissue. Brain Res. Protocols. 10: 156-167 (2003)
143) Tao et al, Functional Genomics: Expression Analysis of E. coli Growing on Minimal and Rich Media. J. Bacteriol. 181(20): 6425-6440 (1999)
144) Iyer et al, Absolute mRNA Levels and Transcriptional Initiation Rates In Saccharomyces Cerv. Proc. Natl. Acad. Sci. US. 93: 5208-5212 (1996)
145) Taniguichi et al, Competitive RT-PCR Elisa: A Rapid Sensitive and Non-Radioactive Method to Quantitate Cytokine mRNA. J. Immun. Methods. 169: 101-109 (1994)
146) Wang et al, Quantitation of mRNA By PCR. Proc. Natl. Acad. Sci. US. 86: 9717-9721 (1989)
147) Kang et al, Cellular Transcriptome Analysis Using A Kinetic PCR Assay. In PCR Applications, Protocols For Functional Genomics. Ed. Innis et al, Acad. Press. Chapter 27: 429-444 (1999)
148) Baker, Control of Poly(A) Length. In Control of Messenger RNA Stability. Ed Belasco et al. Acad. Press. Chapter 15: 367-415 (1993)
149) Olivas et al, The Puf3 Protein Is A Transcript-Specific Regulator of mRNA Degradation In Yeast. Embo Journal. 19(23): 6602-6611 (2000)
150) Decker et al, A Turnover Pathway For Both Stable and Unstable mRNAs In Yeast: Evidence For A Requirement For Deadenylation. Genes and Development. 7: 1632-1643 (1993)
151) Badiee et al, Evaluation of Five Different cDNA Labeling Methods For Microarrays Usion Spike Controls. BMC Biotech. 3(23): (Dec. 11, 2003)
152) Yang et al, Within the Fold: Assessing Differential Expression Measures and Reproducibility In Microarray Assays. Genome Biology 3(11): 0062.1-0062.12 (2002)
153) Schena, Microarray Analysis. Wiley-Liss (2003)
154) Xiang et al, Anime-Modified Random Primers To Label Probes For DNA Microarrays. Nature Biotech. 20: 738-742 (2002)
155) Richter et al, Comparison of Fluorescent Tag DNA Labeling Methods Used For Expression Analysis By DNA Microarrays. Biotechniques. 33: 620-630 (2002)
156) Yu et al, Evaluation and Optimization of Procedures For Target Labeling and Hybridization of cDNA Microarrays. Molecular Vision. 8: 130-137 (2002)
157) 't Hoen et al, Fluorescent Labeling of cRNA For Microarray Applications. Nuc. Acid Res. 31(5): e20 (2003)
158) Bartosiewicz et al, Development Of A Toxicological Gene Array, and Quantitative Assessment Of This Technology. Arch. Biochem. And Bioph. 376(1): 66-73
159) Benes et al, Standardization of Protocols In cDNA Microarray Analysis. Trends in Biochm. Sci. 28: 244-249 (2003)
160) Dombkowski et al, Gene-Specific Dye Bias In Microarray Reference Designs. Febs Letters. 560: 120-124 (2004)
161) Randolph et al, Stability, Specificity, and Fluorescence Brightness of Multiply-Labeled Fluorescent DNA Probes. Nuc. Acid Res. 25(14): 2923-2929 (1997)
162) Cox et al, Possible Sources of Dye-Related Signal Correlation Bias In Two Color DNA Microarray Assays. Anal. Biochem. 331: 243-254 (2004)
163) Wang et al, Optical Properties of ALEXA 488 and CY 5 Immobilized On A Glass Surface. Biotechniques. 38: 127-132 (2005)
164) Naderi et al, Expression Microarray Reproducibility Is Improved By Optimizing Purification Steps In RNA Amplification and Labeling. BMC Genomics. 5(9): (Jan. 30, 2004).
165) Ryan et al, Application and Optimization of Microarray Technologies For Human Postmortem Brain Studies. Biol. Psychiatry. 55: 329-336 (2004)
166) Marchuk et al, Postmortem Stability of Total RNA Isolated From Rabbit Ligament, Tendon, and Cartilage. Biochem. Biophys. Acta. 1379: 171-177 (1998)
167) Bashiardes et al, cDNA Detection and Analysis. Current Opinion In Chem Biol. 5(1): 15-20 (2001)
168) Taylor et al, Recovery and Measurement of Specific RNA Species From Postmortem Brain Tissue: A General Reduction in Alzheimer's Disease Detected By Molecular Hybridization. Exptl. And Molec. Pathology. 44: 111-116 (1986)
169) Luzzi et al, Accurate and Reproducible Gene Expression Profiles From Laser Capture Microdissection, Transcript Amplification, And High Density Oligonucleotide Microarray Analysis. J. Mol. Diagnostics. 5(1): 9-14 (2003)
170) Feldman et al, Advantages of mRNA Amplification For Microarray Analysis. Biotechniques. 33: 906-914 (2002)
171) Baugh et al, Quantitative Analysis of mRNA Amplification By In Vitro Transcription. Nuc. Acid Res. 29(5): e29 (2001)
172) Carninci et al, Thermostabilization and Thermoactivation of Thermolabile Enzymes By Trehalose And Its Application For The Synthesis Of Full Length cDNA. Proc. Natl. Acad. Sci, US. 95: 520-524 (1998)
173) Stears et al, A Novel, Sensitive Detection System For High-Density Microarrays Using Dendrimer Technology. Physiol. Genomics. 3: 93-99 (2000)
174) Rouse et al, Development Of A Microarray Assay That Measures Hybridization Stoichiometry in Moles. Biotechniques. 36: 464-470 (2004)
175) Sendera et al, Expression Profiling With Oligonucleotide Arrays: Technologies and Applications For Neurobiology. Neurochemical Research. 27(10): 1005-1026 (2002)
176) Ramakrishnan et al, An Assessment of Motorola Codelink Microarray Performance For Gene Expression Profiling Applications. Nuc. Acid Res. 30(7): e30 (2002)
177) Shippy et al, Performance Evaluation Of Commercial Short-Oligonucleotide Microarrays And The Impact Of Noise In Making Cross-Platform Correlations. BMC Genomics. 5(61): (Sep. 2, 2004)
178) Lu, Improving The Scaling Normalization For High-Density Oligonucleotide Genechip Expression Microarrays. BMC Bioinformatics. 5(103): (Jul. 29, 2004)
179) Rampal, DNA Arrays, Methods And Protocols. Humana Press Methods In Molecular Biology vol 170 (2001)
180) Zhang et al, Microarray Quality Control. Wiley-Liss (2004)
181) Gautier et al, Alternative Mapping Of Probes To Genes For Affymetrix Chips. BMC Bioinformatics 5(111): (Aug. 14, 2004)
182) Relogio et al, Optimization of Oligonucleotide-Based DNA Microarrays. Nuc. Acid Res. 30(11): e51 (2002)
183) Chen et al, Optimal cDNA Microarray Design Using Expressed Sequence Tags For Organisms With Limited Genomic Information. BMC Bioinformatics. 5(191): (Dec. 7, 2004)
184) Zhang et al, A Model of Molecular Interactions On Short Oligonucleotide Microarrays. Nature Biotech. 21(7): 818-819 (2003)
185) Lemon et al, Theoretical and Experimental Comparisons of Gene Expression Indexes For Oligonucleotide Arrays. Bioinformatics. 18: 1470-1476 (2002)
186) Flavell et al, DNA-DNA Hybridization On Nitrocellulose Filters, 1. General Considerations And Non-Ideal Kinetics. Eur. J. Biochem. 47: 535-543 (1974)
187) Hames et al, Nucleic Acid Hybridization, A Practical Approach. IRL Press, Oxford (1985)
188) Wetmur et al, Kinetics of Renaturation of DNA. J. Mol. Biol. 31:349-370 (1968)
189) GE-Amersham Biosciences Instruction Document 63005460 (080075-00)/Rev. AA/2004-04): Codelink Gene Expression System: Single-Assay Bioarray Hybridization and Detection. (Obtained in Late 2004)
190) Affymetrix Instruction Document 701028 Rev. 5: Section 2 Chapter 3, Eukaryotic Arrays: Washing, Staining, and Scanning. (Obtained in Late 2004)
191) Mygind et al, Determination of PCR Efficiency In Chelex-100 Purified Clinical Samples and Comparison of Real-Time Quantitative PCR and Conventional PCR For Detection Of Chlamydia Pneumoniae. BMC Microbiology 2(17): (Jul. 9, 2002)
192) Korke et al, Large Scale Gene Expression Profiling Of Metabolic Shift Of Mammalian Cells In Culture. J. Biotech 107: 1-17 (2004)
193) Jen et al, Transcriptional Response Of Lymphoblastoid Cells To Ionizing Radiation. Genome Research. 13: 2092-2100 (2003)
194) Bomprezzl et al, Gene Expression Profile In Multiple Sclerosis Patients and Healthy Controls: Identifying Pathways Relevant to Disease. Human Mol. Genetics. 12(17): 2191-2199 (2003)
195) Porter et al, Dissection Of Temporal Gene Signatures Of Affected And Spared Muscle Groups In Dystrophin Deficient (mdx) Mice. Human Mol. Genetics. 12(15): 1813-1821 (2003)
196) Hawkins et al, Gene Expression Differences In Quiescent Versus Regenerating Hair Cells Of Avain Sensory Epithelia: Implications For Human Hearing And Balance Disorders. Human Mol. Genetics. 12(11): 1261-1272 (2003)
197) Yue et al, An Evaluation Of The Performance Of cDNA Microarrays For Detecting Changes In Global mRNA Expression. Nuc. Acid Res. 29(8): e41 (2001)
198) Spencer et al, Muliplex Relative RT-PCR Method For Verification Of Differential Gene Expression. Biotechniques. 27: 1044-1052 (1999)
199) Firestein et al, DNA Microarrays: Boundless Technology Or Bound By Technology? Guidelines For Studies Using Microarray Technology. Arthritis And Rheumatism. 46(4): 859-861 (2002)
200) Melamed et al, Flow Cytometry And Sorting, Second Edition. Wiley-Liss (1990)
201) Birren et al, Genome Analysis, A Lab Manual (vol 1 1997), (vol 2 1998). Cold Spring Harbor Laboratory Press
202) Barker, At The Bench, A Laboratory Navigator. Cold Spring Harbor Laboratory Press (1998)
203) Roskams, Lab REF, A Handbook For Recipes, Reagents, And Other Reference Tools For Use At The Bench. Cold Spring Harbor Laboratory Press (2002)
204) Tracy et al, Detection, Sizing, and Quantitation Of Polyadenylated RNA In The Nanogram-Picogram Range. Biochemistry. 19: 3792-3799 (1980)
205) Crissman et al, Correlated Measurements Of DNA, RNA, And Protein In Individual Cells By Flow Cytometry. Science. 228: 1321-1324 (1985)
206) Sherman, Getting Started With Yeast. In Methods In Enzymology. Academic Press vol 194: 3-21 (1991)
207) Rickwood et al, Gel Electrophoresis Of Nucleic Acids, Second Edition, A Practical Approach. IRL Press, Oxford (1990)
208) Mitchelson et al, Capillary Electrophoresis Of Nucleic Acids: Volume 1 Introduction To Capillary Electrophoresis of Nucleic Acids. Humana Press. Methods In Molecular Biology vol 162 (2001)
209) Graham et al, DNA Sequencing Protocols, Second Edition. Humana Press, Methods In Molecular Biology vol 167 (2001)
210) Mason, Fluorescent And Luminescent Probes For Biological Activity, A Practical Guide To Technology For Quantitative Real-Time Analysis. Academic Press. Biological Technique Series (1993)
211) Pawley, Handbook Of Biological Control Microscopy, Second Edition. Plenum Press (1995)
212) L'Annunziata, Handbook Of Radioactivity Analysis. Academic Press (1998)
213) Dorris et al, Oligodeoxyribonucleotide Probe Accessibility On A Three-Dimensional DNA Microarray Surface And The Effect Of The Hybridization Time On The Accuracy Of Expression Ratios BMC Biotech. 3(6): (Jun. 11, 2003)
214) Peterson et al, The Effect Of Surface Probe Density On DNA Hybridization. Nuc. Acid Res. 29(24): 5163-5168 (2001)
215) Li et al, Selection Of Optimal DNA Oligos For Gene Expression Arrays. Bioinformatics. 17(11): 1067-1076 (2001)

### V. COMMENTS ON CONTENTS OF DISCLOSURE

All patents and other references cited in the specification are indicative of the level of skill of those skilled in the art to which the invention pertains, and are incorporated by reference in their entireties, including any tables and figures, to the same extent as if each reference had been incorporated by reference in its entirety individually. The citation of any publication is for its disclosure prior to the filing date and should not be construed as an admission that the present invention is not entitled to antedate such publication by virtue of prior invention.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to the particular assay or set of assays, and to the manner and materials used for conducting the assays. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention.

Thus, additional embodiments are within the scope of the invention and within the following claims.

1. A method for producing improved particular gene (PG) RNA transcript expression analysis assay results for, a PG RNA transcript expression analysis assay for a cell sample RNA transcript preparation or equivalent nucleic acids derived therefrom, or a PG RNA transcript expression comparison analysis assay for compared cell sample RNA preparations or equivalent nucleic acids derived therefrom, comprising
normalizing the assay measured PG RNA transcript expression results for an analyzed cell sample and the assay measured PG RNA transcript expression comparison results for the compared cell samples or both, for one or more of:
(a) one or more pertinent assay variable-associated unconsidered normalization factors (UNFs) using pertinent assay values for individual UNFs or UNF combinations or both;
(b) one or more pertinent improved considered normalization factor (CNF) assay values whose values are known to be improved, using pertinent assay values for individual CNFs or CNF combinations or both.
wherein said normalizing produces assay results which are known to be improved in normalization and in interpretability relative to such RNA transcript expression assay results and PG RNA transcript expression comparison assay results obtained by prior assay and normalization practices.

2. The method of claim 1, wherein at least one said UNF is utilized.

3. The method of claim 1, wherein at least one said improved CNF is utilized.

4. The method of claim 1, wherein at least one said UNF and at least one said improved CNF is utilized.

5-23. (canceled)

24. The method of claim 1, further comprising identifying one or more UNFs which are pertinent for said assay.

25. (canceled)

26. The method of claim 1, further comprising identifying one or more CNFs which are pertinent for said assay.

27. (canceled)

28. The method of claim 26, further comprising determining that a said CNF is an improved CNF, an invalid CNF, or an uncertain validity CNF

29. (canceled)

30. The method of claim 26, further comprising
(a) determining that the compared cell sample measured total mRNA content per cell or the total number of mRNA molecules per cell (STM) values differ significantly;
(b) determining that the measured difference is not primarily due to a greater number of mRNA molecules from genes which are expressed only in the compared sample which is associated with the larger measured value; and
(c) determining that the difference in compared measured values is not primarily due to an increase in mRNA copies per cell in only one of the compared samples for one or more genes which are expressed in both compared samples,
wherein if (a) and (b) and (c) are true, then said CNF is an invalid CNF.

31. (canceled)

32. The method of claim 26, further comprising
(a) determining for each compared cell sample the total mRNA content per cell or the total number of mRNA molecules per cell (STM); and
(b) comparing the determined values,
wherein if the compared determined values are significantly different then said CNF is a CNF of uncertain validity.

33-38. (canceled)

39. The method of claim 1, wherein said assay is a microarray assay.

40. The method of claim 1, wherein said assay is an RT-PCR assay.

41. The method of claim 1, wherein said assay is a nuclease protection assay.

42. The method of claim 1, wherein said assay is a clone counting or SAGE assay.

43. The method of claim 1, wherein said assay is an ELISA assay.

44. The method of claim 1, wherein said assay is an affinity medium separation assay.

45. The method of claim 44, wherein said affinity medium is hydroxyapatite.

46. (canceled)

47. The method of claim 1, wherein said improved assay result is completely normalized for all assay pertinent UNFs and CNFs.

48. The method of claim 1, wherein said improved assay result has improved normalization for at least one, but less than all, assay pertinent UNFs and assay pertinent CNFs, thereby producing an improved PG assay result which is incompletely normalized for all assay pertinent UNFs and CNFs.

49. The method of claim 1, wherein unconsidered assay variable associated UNFs comprise one or more of the UNFs A•SC, A•SCR, R•SC, R•SCR, PAF, PAFR, MLD, MLDR, PL-HKR, PS-HKR, PSA, PSAR, PSS, PSSR, LLS, LLSR, SBN, SBNR, SSA, SSAR, STM, STMR.

50. The method of claim 1, wherein the prior art known and considered assay variable associated CNFs comprise one or more of the CNFs sampling statistics, sequencing error, C-HKR, spatial, print tip, print plate, intensity scale, AE•SE, AE•SER, AE•AE, AE•AER,

51. The method of clam 1, wherein said assay is a microarray SGDS or DGDS type 1 direct label LPN assay which analyzes cell sample RNA transcripts or their equivalent cDNA or cRNA nucleic acids, and the CNFs comprise one or more of C-HKR, spatial, print tip, print plate, intensity, scale, or the UNFs comprise one or more of A•SC, A•SCR, R•SC, R•SCR, PAF, PAFR, MLD, MLDR, PL-HKR, PS-HKR, PSA, PSAR, PSS, PSSR, or both the CNF and UNF as specified are utilized.

52. The method of claim 1, wherein said assay is a microarray DGSS type 1 direct label LPN assay which analyzes cell sample RNA transcripts or their equivalent cDNA or cRNA nucleic acids, and the CNFs comprise one or more of C-HKR, spatial, print tip, print plate, intensity, scale, or the UNFs comprise one or more of A•SC, R•SC, PAF, PAFR, MLD, MLDR, PL-HKR, PS-HKR, PSA, PSAR, PSS, PSSR, or both the CNF and UNF as specified are utilized.

53. The method of claim 1, wherein said assay is a microarray SGDS or DGDS type 2 direct label LPN assay which analyzes cell sample RNA transcripts or their equivalent cDNA or cRNA nucleic acids, and the CNFs comprise one or more of C-HKR, spatial, print tip, print plate, intensity, scale, or the UNFs comprise one or more of A•SC, A•SCR, R•SC, R•SCR, PAF, PAFR, PL-HKR, PS-HKR, LLS, LLSR, or both the CNF and UNF as specified are utilized.

54. The method of claim 1, wherein said assay is a microarray DGSS type 2 direct LPN assay which analyzes cell sample RNA transcripts or their equivalent cDNA or cRNA nucleic acids, and the CNFs comprise one or more of C-HKR, spatial, print tip, print plate, intensity, scale, or the UNFs comprise one or more of A•SC, R•SC, PAF, PAFR, PL-HKR, PS-HKR, LLS, LLSR, or both the CNF and UNF as specified are utilized.

55. The method of claim 1, wherein said assay is a microarray SGDS or DGDS type 1 indirect LPN assay which analyzes cell sample RNA transcripts or their equivalent cDNA or cRNA nucleic acids, and the CNFs comprise one or more of C-HKR, spatial, print tip, print plate, intensity and scale, or the UNFs comprise one or more of A•SC, A•SCR, R•SC, R•SCR, PAF, PAFR, MLD, MLDR, PL-HKR, PS-HKR, SBN, SBNR, SSA, SSAR, or both the CNF and UNF as specified are utilized.

56. The method of claim 1, wherein said assay is a microarray DGSS type 1 indirect LPN assay which analyzes cell sample RNA transcripts or their equivalent cDNA or cRNA nucleic acids, and the CNFs comprise one or more of C-HKR, spatial, print tip, print plate, intensity, scale, or the UNFs comprise one or more of A•SC, R•SC, PAF, PAFR, MLD, MLDR, PL-HKR, PS-HKR, SBN, SBNR, SSA, SSAR, or both the CNF and UNF as specified are utilized.

57. The method of claim 1, wherein said assay is a microarray SGDS or DGDS type 2 indirect LPN assay which analyzes cell sample RNA transcripts or their equivalent cDNA or cRNA nucleic acids, and the CNFs comprise one or more of C-HKR, spatial, print tip, print plate, intensity, scale, or the UNFs comprise one or more of A•SC, A•SCR, R•SC, R•SCR, PAF, PAFR, PL-HKR, PS-HKR, SBN, SBNR, LLS, LLSR, or both the CNF and UNF as specified are utilized.

58. The method of claim 1, wherein said assay is a microarray DGSS type 2 indirect LPN assay which analyzes cell sample RNA transcripts or their equivalent cDNA or cRNA nucleic acids, and the CNFs comprise one or more of C-HKR, spatial, print tip, print plate, intensity, scale, or the UNFs comprise one or more of A•SC, R•SC, PAF, PAFR, PL-HKR, PS-HKR, SBN, SBNR, LLS, LLSR, or both the CNF and UNF as specified are utilized.

59-77. (canceled)

78. The method of claim 1, wherein said assay is a non-microarray nuclease protection SGDS type 1 or type 2 direct or indirect LPN assay which analyzes cell sample RNA transcripts or equivalent cDNA or cRNA nucleic acids, and the CNFs comprise one or more of C-HKR, intensity, or the UNFs comprise one or more of A•SC, A•SCR, R•SC, R•SCR, PAF, PAFR, MLD, MLDR, or both the CNF and UNF as specified are utilized.

79. The method of claim 1, wherein said assay is a non-microarray nuclease protection DGDS type 1 direct LPN assay which analyzes cell sample RNA transcripts or equivalent cDNA or cRNA nucleic acids, and the CNFs comprise one or more of C-HKR, intensity, or the UNFs comprise one or more of A•SC, A•SCR, R•SC, R•SCR, PAF, PAFR, MLD, MLDR, PL-HKR, PS-HKR, PSA, PSAR, PSS, PSSR, or both the CNF and UNF as specified are utilized.

80. The method of claim 1, wherein said assay is a non-microarray nuclease protection DGDS type 2 direct LPN assay which analyzes cell sample RNA transcripts or equivalent cDNA or cRNA nucleic acids, and the CNFs comprise one or more of C-HKR, intensity, or the UNFs comprise one or more of A•SC, A•SCR, R•SC, R•SCR, PAF, PAFR, PL-HKR, PS-HKR, LLS, LLSR, or both the CNF and UNF as specified are utilized.

81. The method of claim 1, wherein said assay is a non-microarray nuclease protection DGDS type 1 indirect LPN assay which analyzes cell sample RNA transcripts or equivalent cDNA or cRNA nucleic acids, and the CNFs comprise one or more of C-HKR, intensity, or the UNFs comprise one or more of A•SC, A•SCR, R•SC, R•SCR, PAF, PAFR, MLD, MLDR, PL-HKR, PS-HKR, SBN, SBNR, SSA, SSAR, or both the CNF and UNF as specified are utilized.

82. The method of claim 1, wherein said assay is a non-microarray nuclease protection DGDS type 2 indirect LPN assay which analyzes cell sample RNA transcripts or equivalent cDNA or cRNA nucleic acids, and the CNFs comprise one or more of C-HKR, intensity, or the UNFs comprise one or more of A•SC, A•SCR, R•SC, R•SCR, PAF, PAFR, PL-HKR, PS-HKR, SBN, SBNR, LLS, LLSR, or both the CNF and UNF as specified are utilized.

83. The method of claim 1, wherein said assay is a non-microarray nuclease protection DGSS type 1 direct LPN assay which analyzes cell sample RNA transcripts or equivalent cDNA or cRNA nucleic acids, and the CNFs comprise one or more of C-HKR, intensity, or the UNFs comprise one or more of A•SC, A•SCR, R•SC, R•SCR, PAF, PAFR, MLD, MLDR, PL-HKR, PS-HKR, PSA, PSAR, PSS, PSSR, or both the CNF and UNF as specified are utilized.

84. The method of claim 1, wherein said assay is a non-microarray nuclease protection DGSS type 2 direct LPN assay which analyzes cell sample RNA transcripts or equivalent cDNA or cRNA nucleic acids, and the CNFs comprise one or more of C-HKR intensity, or the UNFs comprise one or more of A•SC, A•SCR, R•SC, R•SCR, PAF, PAFR, PL-HKR, PS-HKR, LLS, LLSR, or both the CNF and UNF as specified are utilized.

85. The method of claim 1, wherein said assay is a micro-array nuclease protection DGSS type 1 indirect LPN assay which analyzes cell sample RNA transcripts or equivalent cDNA or cRNA nucleic acids, and the CNFs comprise one or more of C-HKR, intensity, or the UNFs comprise one or more of A•SC, A•SCR, R•SC, R•SCR, PAF, PAFR, PL-HKR, PS-HKR, SBN, SBNR, SSA, SSAR, or both the CNF and UNF as specified are utilized.

86. The method of claim 1, wherein said assay is a non-microarray nuclease protection DGSS type 2 indirect LPN assay which analyzes cell sample RNA transcripts or equivalent cDNA or cRNA nucleic acids, and the CNFs comprise one or more of C-HKR, intensity, or the UNFs comprise one or more of A•SC, A•SCR, R•SC, R•SCR, PAF, PAFR, PL-HKR, PS-HKR, SBN, SBNR, LLS, LLSR, or both the CNF and UNF as specified are utilized.

87. The method of claim 1, wherein said assay is a non-microarray RT-PCR SGDS, DGDS, or DGSS, assay which analyzes cell sample RNA transcripts or equivalent cDNA or cRNA nucleic acids, and the CNFs comprise one or more of AE•SE, AE•SER, AE•AE, AE•AER, or the UNFs comprise one or more of A•SC, A•SCR, R•SC, R•SCR, PAF, PAFR, or both the CNF and UNF as specified are utilized.

88. The method of claim 1, wherein said assay is a non-microarray RT-PCR SGDS, DGDS, or DGSS assay which analyzes cell sample RNA transcripts or equivalent cDNA or cRNA nucleic acids, and one or more exogenous and/or endogenous S RNA transcripts or equivalent cDNA or cRNA nucleic acids, and the CNFs comprise one or more of AE•SE, AE•SER, AE•AE, AE•AER, or the UNFs comprise one or more of A•SC, A•SCR, R•SC, R•SCR, PAF, PAFR, or both the CNF and UNF as specified are utilized.

89-90. (canceled)

91. The method of claim 1, wherein said improved PG RNA transcript expression analysis assay results produced include one or more or all of the following:
(a) an assay measured and normalized relative or absolute value for the number of RNA transcript per sample cell, for one or more or all of the different said assay detectable PG RNA transcripts which are present in the analyzed cell sample RNA transcript preparation;
(b) a normalized differential gene expression ratio (N-DGER) value for a different gene same cell sample (DGSS)RNA transcript expression analysis assay comparison of different particular gene RNA transcripts which are present in the same cell sample RNA transcript preparation;
(c) a normalized differential gene expression ratio (N-DGER) value for a same gene different cell sample (SGDS) RNA transcript expression analysis assay comparison of the same PG RNA transcripts which are present in different cell sample RNA transcript preparations;
(d) a normalized differential gene expression ratio (N-DGER) value for a different gene different cell sample (DGDS) RNA transcript expression analysis assay comparison of different PG RNA transcripts which are present in different cell sample RNA transcript preparations;
(e) an assay measured and normalized relative or absolute value for the RN value for one or more or all of the different PG RNA transcripts which are present in an aliquot of a cell sample RNA transcript preparation; and
(f) a combination of one or more or all possible, SGDS, DGDS, and DGSS particular gene RNA transcript comparison N-DGER values, and PG relative or absolute RN or abundance values, from one or more different RNA transcript expression analysis assays.

92-97. (canceled)

98. The method of claim 1, wherein, the gene expression RNA transcript expression analysis assay of a cell sample RNA transcript preparation or equivalent cDNA or cRNA nucleic acids, utilizes one or more exogenous RNA or DNA transcript artificial housekeeping gene standards or one or more valid endogenous RNA transcript true housekeeping gene standards, to produce for one or more non-housekeeping PGs in the assay one or more of:
(a) improved relative or absolute values or both for a PG abundance or number of RNA transcripts per sample cell which is present in the analyzed cell sample,
(b) improved relative or absolute values or both for the number of PG RNA transcripts per sample cell haploid DNA content; and
(c) improved relative or absolute values or both for a PG RN which is associated with an aliquot of analyzed cell sample RNA.

99. The method of claim 98, wherein one or more artificial housekeeping gene standards are utilized.

100. The method of claim 99, wherein one or more one or more valid endogenous true housekeeping genes are utilized.

101-103. (canceled)

104. The method of claim 98, wherein one or more artificial housekeeping genes (AHG) are used to facilitate the determination of assay pertinent UNF and CNF values, comprising
a) determining the number of each cell sample's cell equivalents (CE) present in the cell sample nucleic acid sample being analyzed in the assay;
b) adding a known number of molecules for each of one or more particular RNA or DNA standards to each said cell sample nucleic acid sample being analyzed in the assay, thereby producing in each cell sample nucleic acid sample being analyzed in the assay one or more artificial housekeeping gene (AHG) particular RNAs or DNAs whose copy per cell or abundance value is known;
c) performing the assay and producing raw assay results for each particular cell sample particular gene and particular AHG; and
d) utilizing the raw assay results for at least one particular standard AHG and the known abundance value for the particular standard AHG in the sample and the known true differential gene expression ratio value for the particular standard AHG in compared cell samples in determining the assay values for UNFs and CNFs which are pertinent for the assay.

105. The method of claim 104, further comprising
utilizing the determined UNF values or CNF values or both to normalize the cell sample particular gene assay results.

106. The method of claim 98, wherein a plurality of different AHG standards are used.

107-114. (canceled)

115. The method of claim 98, wherein said assay comprises an assay selected from the group consisting of a) a microarray assay,
b) a DOT blot assay,
c) a northern blot assay,
d) a nuclease protection assay,
e) an RT-PCR assay, and
f) a clone counting or SAGE assay.

116-134. (canceled)

135. The method of claim 1, wherein the cell sample RNA transcript preparation analyzed or the cell sample RNA transcript preparations compared are derived from one or more normal or diseased or pathologic cell samples of the same eukaryotic species or strain which have been treated with the same or different physical or chemical stimuli or other treatment.

136-151. (canceled)

152. The method of claim 1, wherein said analyzed cell sample RNA transcripts or equivalent nucleic acids derived therefrom represent cell sample total RNA transcripts.

153. The method of claim 1, wherein said analyzed cell sample RNA transcripts or equivalent nucleic acids derived therefrom represent cell sample isolated mRNA transcripts.

154-170. (canceled)

171. The method of claim 1, wherein said analyzed cell sample RNA transcripts or equivalent nucleic acids derived therefrom represent foreign prokaryotic or eukaryotic cell total RNA, mRNA, miRNA, siRNA, snoRNA, rRNA, or tRNA transcripts or combinations thereof which are present in a cell sample total RNA or isolated RNA preparation.

172-173. (canceled)

174. The method of claim 1, wherein the cell sample gene expression analysis assay of one or more cell sample RNA transcript preparations or equivalent nucleic acids derived therefrom, incorporates one or more of the following assay design solutions,
(a) as few assay pertinent UNFs as possible;
(b) as many assay pertinent UNF assay values as possible equal one;
(c) as few CNFs as possible are assay pertinent;
(d) as many assay pertinent CNF assay values as possible equal one;
(e) the occurrence of CNF and UNF related false negative particular gene assay results is minimized or eliminated;
(f) the use in the assay of one or more exogenous standard artificial housekeeping gene (AHG) RNAs or DNAs in order to simplify and improve the determination of the assay values for one or more assay pertinent CNFs or one or more assay pertinent UNFs or both;
(g) the use in the assay of one or more exogenous S RNAs or DNAs in order to simplify and improve the determination of the assay values for one or more assay pertinent CNFs or one or more assay pertinent UNFs or both;
(h) the identification of and the use in the assay of one or more true housekeeping gene RNA transcripts which are endogenous to the cell sample or cell samples, in order to simplify and improve the determination of the assay values for one or more assay pertinent CNFs or one or more assay pertinent UNFs or both; and
(i) the use of one or more AHG or true housekeeping gene or both RNA or DNA transcripts whose abundance values are known, in order to determine the abundance values of one or more non-control PG RNA transcripts in a cell sample.

175-186. (canceled)

187. A method for producing improved microarray assay measured SGDS, DGDS, or DGSS particular gene RNA transcript expression comparison N-DGER values which are known to be improved in normalization and interpretation relative to prior art microarray assay produced gene expression comparison N-DGER values, comprising
utilizing a design solution combination in said assay wherein
(a) said design solution combination is selected from the group consisting of the design solution combinations presented in Tables 54-60, 75-81, and 100-102; or
(b) the design solution combination is selected from the group consisting of the design solution combinations presented in Tables 61-69, and 82-90.

188-189. (canceled)

190. A method for producing improved nuclease protection assay measured SGDS, DGDS, or DGSS particular gene RNA transcript expression comparison N-DGER values which are known to be improved in normalization and interpretation, relative to prior art nuclease protection assay produced particular gene expression comparison N-DGER values, comprising
utilizing in said assay a design solution combination selected from the group consisting of the design solution combinations presented in Table 95.

191. A method for producing improved RT-PCR assay measured SGDS, DGDS, or DGSS particular gene RNA transcript expression comparison N-DGER values which are known to be improved in normalization and interpretation, relative to prior art RT-PCR assay produced particular gene expression comparison N-DGER values, comprising
utilizing in said assay a design solution selected from the group consisting of the design solution combinations presented in Table 97.

192-195. (canceled)

196. An assay kit for improving or validating or calibrating a particular gene (PG) RNA transcript expression analysis or PG transcript comparison analysis assay or both for a cell sample RNA transcript preparation or equivalent nucleic acids derived therefrom, comprising
a packaged reagent set comprising
at least one reagent for carrying out said assay; and
instructions for performing said assay with improved normalization, or a quantity of at least one improved normalization reagent for obtaining said improved normalization, or both.

197. The assay kit of claim 196, comprising said instructions for performing said assay with improved normalization.

198. The assay kit of claim 196, comprising said improved normalization reagent.

199. The assay kit of claim of 196, comprising
both said instructions and a quantity of said improved normalization reagent.

200. The assay kit of claim 196, wherein said normalization reagent comprises at least one defined RNA or DNA.

201. The assay kit of claim 200, wherein said defined RNA or DNA comprises at least one artificial housingkeeping gene (AHG), wherein use of said AHG improves determination of one or more assay pertinent UNFs or CNFs or both.

202. The assay kit of claim claim 201, comprising both said instructions and said at least one AHG.

203. The assay kit of claim 196, wherein said improved normalization reagent comprises
a quantity of at least one cell sample total RNA or isolated mRNA for which is known characteristic data selected from the group consisting of:
a) the mass amount of cell sample total RNA per cell;
b) the mass amount of cell sample mRNA per cell;
c) the number of mRNA transcripts per cell, for each particular RNA sample;
d) both a) and b);
e) both a) and c);
f) both b) and c);
g) all of a) and b) and c).

204-210. (canceled)

211. The assay kit of claim 196, wherein said improved normalization reagent comprises
reagents for determining quantitative values for any 1, 2, 3, 4, or 5 of:
the mass of total DNA per intact cell,
the total mass of DNA present in the intact cell sample aliquot which is analyzed in the assay,
a cell sample's mass amount of total RNA per intact cell or mRNA per intact cell or both,
the number of mRNA transcripts per intact cell, and
the number of RNA molecules per cell in the cell sample for one or more PGs.

212-213. (canceled)

214. The assay kit of claim 196, wherein said improved normalization reagent comprises
reagents for determining quantitative values for one or more of the following:
the mass amount of total cell sample cDNA LPN or cell sample cRNA LPN per intact cell or both, for each cell sample of interest,
the mass amount of total cell sample cDNA LPN or cRNA LPN or both which is analysed in an assay,
the number of cell sample cDNA or cRNA cell equivalents (CE) which are analysed in an assay,
the cDNA or cRNA associated sample cell number (SC) value or both, for each assayed cell sample,
the cell sample comparison cDNA or cRNA SCR value or both for each cell sample assay comparison, and
the number of cDNA or cRNA transcripts per CE for one or more PGs in the cell sample cDNA or cRNA preparation or both.

215-216. (canceled)

217. The assay kit of claim 196, wherein said improved normalization reagent comprises
a quantity of at least one of:
RNA or DNA oligonucleotide which is improved characterized RNA or DNA, or improved synthesis RNA or DNA, or both,
modified RNA or DNA oligonucleotide,
RNA or DNA analog oligonucleotide,
wherein said oligonucleotide is improved in characterization or synthesis or both, and where said oligonucleotide is associated with normalization improvement for said assay.

218. The assay kit of claim 217, further comprising said instructions.

219. The assay kit of claim 196, wherein said improved normalization reagent comprises
one or more reagents for isolating RNA or DNA or both from a cell sample and determining quantitative values for one or more of:
the cell sample's mass amount of total RNA per intact cell,
the cell sample's mass amount of mRNA per intact cell,
the cell sample's mass amount of total DNA per intact cell,
the mass amount of DNA present in the intact cell sample aliquot which is analysed in the assay, and
the number of mRNA transcripts per intact cell for said cell sample.

220-240. (canceled)

241. The assay kit of claim 196, comprising a system which comprises one or more of the following
a) an oligonucleotide microarray system;
b) a cDNA microarray system;
c) a clone counting or SAGE system;
d) a nuclease protection assay system;
e) a RT-PCR system; or
f) a gene expression analysis system;

242-270. (canceled)

271. A method for evaluating the performance of a gene expression analysis assay, comprising
identifying the pertinent UNFs and CNFs which are associated with the assay;
identifying the normalization assumptions necessary for the valid normalization of assay pertinent CNF values by prior art methods;
determining the assay values for the pertinent UNFs;
determining the assay pertinent CNF values;
normalizing the cell sample and standard PG raw assay results for the determined pertinent UNF and CNF values;
determining quantitative assay metric values for the assay results; and
compare the resulting quantitative assay metric values for the assay with quantititative assay metric values for one or more different assays or one or more standards to evaluate the performance of the assay.

272. The method of claim 271, wherein assay values for pertinent UNFs are determined by improved normalization methods

273. (canceled)

274. The method of claim 271, further comprising
developing nucleic acid test materials comprising cell sample and standard nucleic acid test materials which assist in providing improved UNF and CNF normalization of assay results.

275-283. (canceled)

284. The method of claim 271, wherein improved normalization is utilized to normalize the assay results for pertinent UNFs or to validly normalize the assay results for pertinent CNFs, or both.

285. A method for producing an improved assay kit or assay analysis system, comprising
utilizing a method of claim 271 to evaluate the performance of a gene expression or gene expression comparison analysis system or assay kit of interest; and
identifying a kit or system having desired quantitative assay or system metrics; and
making the identified kit or system.

286. The method of claim 285, further comprising
utilizing a method of claim 271 to evaluate the performance of said kit or system which has been modified;
comparing the performance results of the modified and unmodified kit or system to identify desirable modifications which improve the performance of said kit or system; and
incorporate one or more desired modifications into the kit or system to provide an improved kit or system.

287. A method for producing improved application results, comprising
utilizing improved assay results produced by the method of claim 1 in a an application to produce improved first order application results.

288. The method of claim 287, wherein said improved first order application results comprise improved results of an application selected from the group consisting of
(a) a data analysis and data mining analysis method;
(b) a gene expression profile measurement and identification method for normal, pathologic, or diseased cell samples and combinations thereof;
(c) a bioactive and pharmaceutical candidate or biomarker identification and discovery method;
(d) a systems biology analysis method;
(e) a toxic compound identification and discovery method;
(f) a method for developing gene expression based diagnostic test methods; and
(g) a quality assurance and quality control method for a gene expression analysis application or a method for discovery and identification of toxic compounds, drugs, or bioactive molecules, or combinations thereof.

289-294. (canceled)
